(19) United States
(12) Patent Application Publication
(10) Pub. No.: US 2013/0261077 A1
(43) Pub. Date: Oct. 3, 2013

(54) MANNOSE DERIVATIVES FOR TREATING BACTERIAL INFECTIONS

(71) Applicant: VERTEX PHARMACEUTICALS INCORPORATED, Cambridge, MA (US)

(72) Inventors: Youssef Laafiret Bennani, Lorraine (CA); Caroline Cadilhac, Montreal (CA); Sanjoy Kumar Das, Pierrefonds (CA); Evelyne Dietrich, Laval (CA); Michel Gallant, Pierrefonds (CA); Bingcan Liu, Montreal (CA); Oswy Z. Pereira, Kirkland (CA); Yeeman K. Ramtohul, Pierrefonds (CA); T. Jagadeeswar Reddy, Pierrefonds (CA); Louis Vaillancourt, Mascouche (CA); Constantin Yannopoulos, Notre-Dame de I'lle Perrot (CA); Frederic Vallee, Montreal (CA)

(73) Assignee: VERTEX PHARMACEUTICALS INCORPORATED, Cambridge, MA (US)

(21) Appl. No.: 13/787,418

(22) Filed: Mar. 6, 2013

Related U.S. Application Data
(60) Provisional application No. 61/621,776, filed on Apr. 9, 2012.
(60) Provisional application No. 61/607,778, filed on Mar. 7, 2012.

(51) Int. Cl. C07H 7/04; C07H 3/04; C07H 7/06

(52) U.S. Cl. CPC C07H 7/04; C07H 7/06; C07H 3/04
USPC 514/53; 536/1.11; 514/23; 536/53; 536/55; 536/122; 536/18.7; 536/54; 536/123.13

(57) ABSTRACT

The present invention relates to compounds useful for the treatment or prevention of bacteria infections. These compounds have formula I:

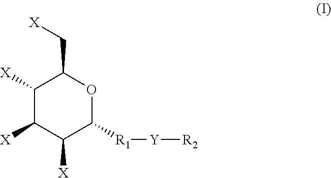

The invention also provides pharmaceutically acceptable compositions containing the compounds and methods of using the compositions in the treatment of bacteria infections. Finally, the invention provides processes for making compounds of the invention.

### CROSS REFERENCE TO RELATED APPLICATIONS

This present invention claims the benefit, under 35 U.S.C. §119, of U.S. Provisional Application No. 61/607,778, filed Mar. 7, 2012; and U.S. Provisional Application No. 61/621,776, filed Apr. 9, 2012; the entire contents of each of the above applications being incorporated herein by reference.

### DESCRIPTION OF THE FIGURES

FIG. 1: X-ray powder diffractogram of Compound 48

FIG. 2: Thermal gravimetric analysis (TGA) trace of Compound 48

### BACKGROUND OF THE INVENTION

Inflammatory bowel disease (IBD) is a complex chronic inflammatory disorder, with the two more common forms being ulcerative colitis (UC) and Crohn's disease (CD). IBD is a multifactorial disease that results from a combination of predisposing genetic factors, environmental triggers, dysbiosis of the gastrointestinal microbiota and an inappropriate inflammatory response (Man et al., 2011, Nat Rev Gastroenterol Hepatol, March, 8(3):152-68).

Several studies on fecal and mucosa-associated bacterial communities have shown that the microbiota of patients with Crohn's disease (CD) differ from those of healthy controls, as well as those of patients with ulcerative colitis (UC). Although the reported changes are not always consistent, numbers of Escherichia coli are generally increased, whereas Firmicutes are scarcer in CD patients (Peterson et al., 2008, Cell Host Microbe, 3: 17-27; Frank et al., 2007, Proc. Natl. Acad. Sci., 104:13780-13785). Whether these changes are causative factors or consequences of inflammation, it remains controversial. To date, several pathogens have been proposed as causative agents. In particular, adherent-invasive E. coli (AIEC) has been reported to be more prevalent in CD patients than in controls in several countries (United Kingdom, France and the USA) (Darfeuille-Michaud et al., 2004, Gastroenterology, 127:412-421; Martinez-Medina et al., 2009, Inflamm Bowel Dis., 15:872-882). AIEC strains have been isolated from ileal lesions in ˜35% of CD patients compared to ˜5% of healthy subjects. One of the features of AIEC is their ability to adhere and invade epithelial cells. It is known from various models that the binding of adhesins expressed on the bacterial cell surface to defined glycosylated receptors on the host tissue surface is considered to be an initial and critical step in pathogenesis, then opening a new avenue for therapy such as blocking the interaction between type 1 pili and CEACAM6, a known host receptor for FimH (Barnich et al., 2007, J. Clin. Invest., 117:1566-1574; Carvalho et al., 2009, JEM, vol. 206, no. 10, 2179-2189). Therefore, inhibition of adhesion, and consequently intracellular replication of AIEC in epithelial cells, may prevent establishment of a sub-mucosal infection leading to mucosal inflammation and epithelial barrier disruption.

It has also been demonstrated recently that FimH antagonists are potentially effective in treating urinary tract infections (J. Med. Chem. 2010, 53, 8627-8641).

### SUMMARY OF THE INVENTION

The present invention provides compounds useful for the treatment or prevention of bacteria infections, such as urinary tract infection (UTI) and inflammatory bowel diseases (IBD) or a pharmaceutically acceptable salt.

These compounds have Formula (I):

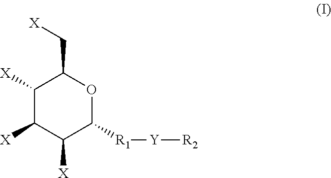

wherein:
X is —H, halogen, (C₁-C₆)alkyl, —NR₅R₆, —SR₇, or —OR₇;
Y is absent or a C₁-C₁₀ aliphatic wherein up to four methylene units of the C₁-C₁₀ aliphatic can be optionally replaced with —NR₈, —O—, —S—, —C(O)—, —S(O)—, or —SO₂—; Y is optionally substituted with 1-2 occurrences of halogen, OH, C₃₋₆cycloalkyl or C₁₋₆aliphatic;
R₁ is cycloalkyl, heterocyclyl, aryl, or heteroaryl; each optionally substituted with one or more R₃ or R_{3A} groups; and
R₂ is —H, or alkyl, cycloalkyl, heterocyclyl, aryl, aralkyl, or heteroaryl; each optionally substituted with one or more R₃ or R_{3B} groups;
R₃ halogen, —CN, NO₂, cycloalkyl, heterocyclyl, aryl, aralkyl, or heteroaryl or a C₁-C₁₀ aliphatic wherein up to four methylene units of the C₁-C₁₀ aliphatic can be optionally replaced with —NR₄, —O—, —S—, —C(O)—, —S(O)—, —SO₂—, or —P(O)—; each R₃ is optionally substituted with one or more R₄ or R_{4A} groups;
R_{3A} is a C₁-C₁₀aliphatic wherein up to four methylene units of the C₁-C₁₀ aliphatic can be optionally replaced with —NR₄, —O—, —S—, —C(O)—, —S(O)—, —SO₂—, or —P(O)—; alkyl, alkenyl, alkynyl, cycloalkyl, or heterocyclyl; each optionally substituted with one or more R₄ or R_{4A} groups;
R_{3B} is aryl, aralkyl, or heteroaryl; R_{3B} is optionally substituted with one or more R₄ or R_{4A} groups;
R₄ is —H, or optionally substituted C₁-C₆ alkyl, C₁-C₆ alkenyl, C₁-C₆ alkynyl, cycloalkyl, heterocyclyl, aryl or heteroaryl;
R_{4A} is halogen, CN, NO₂, or a C₁-C₁₀ aliphatic wherein up to four methylene units of the C₁-C₁₀ aliphatic can be optionally replaced with —NR₄, —O—, —S—, —C(O)—, —S(O)—, —SO₂—, or —P(O)—; each R_{4A} is optionally substituted with 0-3 halo;
R₅ and R₆ are each independently —H, optionally substituted alkyl, alkenyl, alkynyl, cycloalkyl, heterocyclyl, aryl or heteroaryl, —C(O)R₉, —C(O)NHR₉, or —C(O)OR₉;
R₇ is —H, optionally substituted alkyl, alkenyl, alkynyl, cycloalkyl, heterocyclyl, heteroaryl or aryl, —C(O)R₉, or —C(O)NHR₉;
R₈ is —H, optionally substituted alkyl, alkenyl, alkynyl, cycloalkyl, heterocyclyl, heteroaryl, aryl, or —C(O)R₉;
R₉ is —H, optionally substituted alkyl, cycloalkyl, heterocyclyl, aryl or heteroaryl;
R₁₀ is —H, —OH, halogen, or optionally substituted C₁-C₆ alkyl, C₁-C₆ alkenyl, C₁-C₆ alkynyl, cycloalkyl, heterocyclyl, aryl or heteroaryl;
m is 0, 1 or 2; and
n is 0, 1, 2, 3, or 4.

The present invention also provides a composition comprising the compound described herein, and a pharmaceutically acceptable carrier, adjuvant, or vehicle.

The present invention also provides a method of treating or preventing bacteria infection in a subject, comprising administering to the subject an effective amount of the compound or the composition described herein. The present invention also provides processes for making compounds of the invention.

### DETAILED DESCRIPTION OF THE INVENTION

The present invention relates to compounds useful for the treatment or prevention of bacteria infections, such as urinary tract infection (UTI) and inflammatory bowel diseases (IBD).

One embodiment provides a compound of Formula (I), or a pharmaceutically acceptable salt thereof:

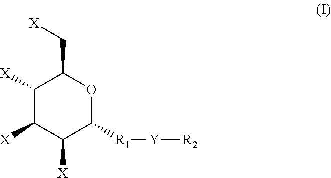

wherein:
X is —H, halogen, (C₁-C₆)alkyl, —NR₅R₆, —SR₇, or —OR₇;
Y is absent or a C₁-C₁₀ aliphatic wherein up to four methylene units of the C₁-C₁₀ aliphatic can be optionally replaced with —NR₈, —O—, —S—, —C(O)—, —S(O)—, or —SO₂—; Y is optionally substituted with 1-2 occurrences of halogen, OH, C₃₋₆cycloalkyl or C₁₋₆aliphatic;
R₁ is cycloalkyl, heterocyclyl, aryl, or heteroaryl; each optionally substituted with one or more R₃ or R_{3A} groups; and
R₂ is —H, or alkyl, cycloalkyl, heterocyclyl, aryl, aralkyl, or heteroaryl; each optionally substituted with one or more R₃ or R_{3B} groups;
R₃ halogen, —CN, NO₂, cycloalkyl, heterocyclyl, aryl, aralkyl, or heteroaryl or a C₁-C₁₀ aliphatic wherein up to four methylene units of the C₁-C₁₀ aliphatic can be optionally replaced with —NR₄, —O—, —S—, —C(O)—, —S(O)—, —SO₂—, or —P(O)—; each R₃ is optionally substituted with one or more R₄ or R_{4A} groups;
R_{3A} is a C₁-C₁₀aliphatic wherein up to four methylene units of the C₁-C₁₀ aliphatic can be optionally replaced with —NR₄, —O—, —S—, —C(O)—, —S(O)—, —SO₂—, or —P(O)—; alkyl, alkenyl, alkynyl, cycloalkyl, or heterocyclyl; each optionally substituted with one or more R₄ or R_{4A} groups;
R_{3B} is aryl, aralkyl, or heteroaryl; R_{3B} is optionally substituted with one or more R₄ or R_{4A} groups;
R₄ is —H, or optionally substituted C₁-C₆ alkyl, C₁-C₆ alkenyl, C₁-C₆ alkynyl, cycloalkyl, heterocyclyl, aryl or heteroaryl;
R_{4A} is halogen, CN, NO₂, or a C₁-C₁₀ aliphatic wherein up to four methylene units of the C₁-C₁₀ aliphatic can be optionally replaced with —NR₄, —O—, —S—, —C(O)—, —S(O)—, —SO₂—, or —P(O)—; each R_{4A} is optionally substituted with 0-3 halo;
R₅ and R₆ are each independently —H, optionally substituted alkyl, alkenyl, alkynyl, cycloalkyl, heterocyclyl, aryl or heteroaryl, —C(O)R₉, —C(O)NHR₉, or —C(O)OR₉;
R₇ is —H, optionally substituted alkyl, alkenyl, alkynyl, cycloalkyl, heterocyclyl, heteroaryl or aryl, —C(O)R₉, or —C(O)NHR₉;
R₈ is —H, optionally substituted alkyl, alkenyl, alkynyl, cycloalkyl, heterocyclyl, heteroaryl, aryl, or —C(O)R₉;
R₉ is —H, optionally substituted alkyl, cycloalkyl, heterocyclyl, aryl or heteroaryl;
R₁₀ is —H, —OH, halogen, or optionally substituted C₁-C₆ alkyl, C₁-C₆ alkenyl, C₁-C₆ alkynyl, cycloalkyl, heterocyclyl, aryl or heteroaryl;
m is 0, 1 or 2; and
n is 0, 1, 2, 3, or 4.

In some embodiments, R₁ is not indole or triazole and the compound of Formula (I) cannot have a structure selected from the group consisting of:

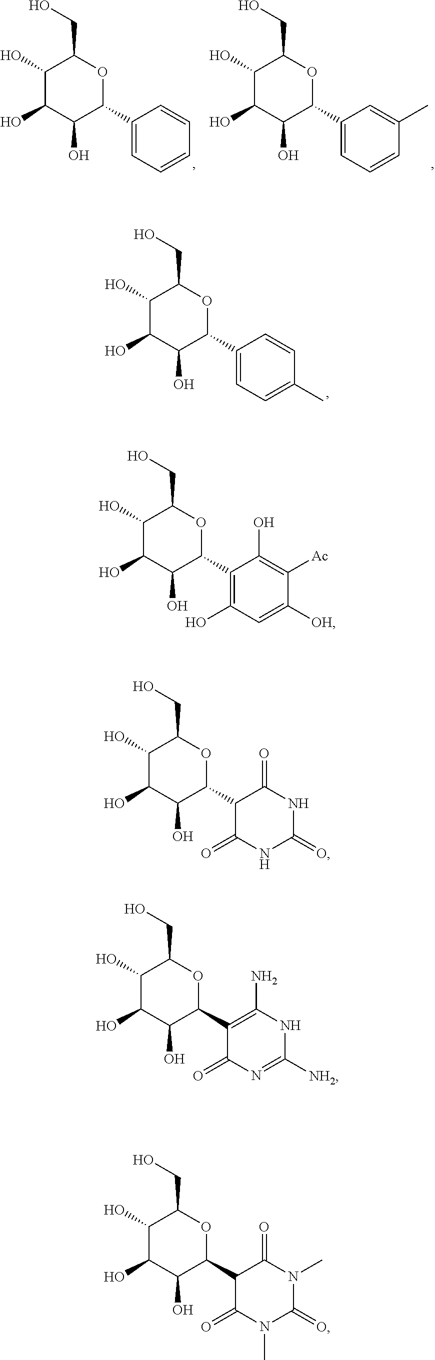

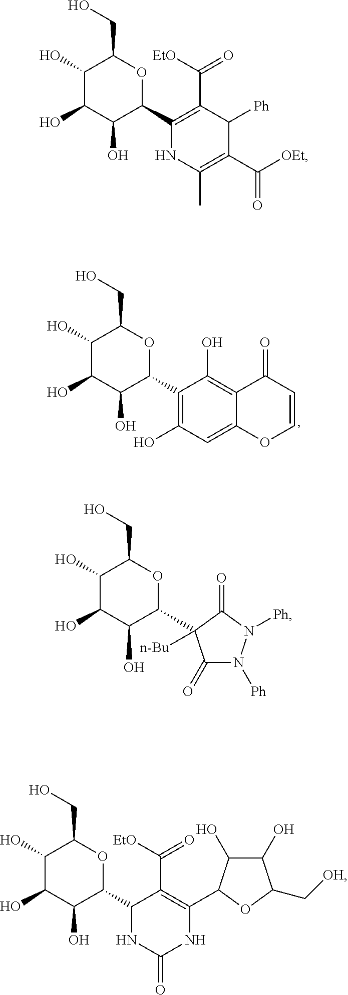

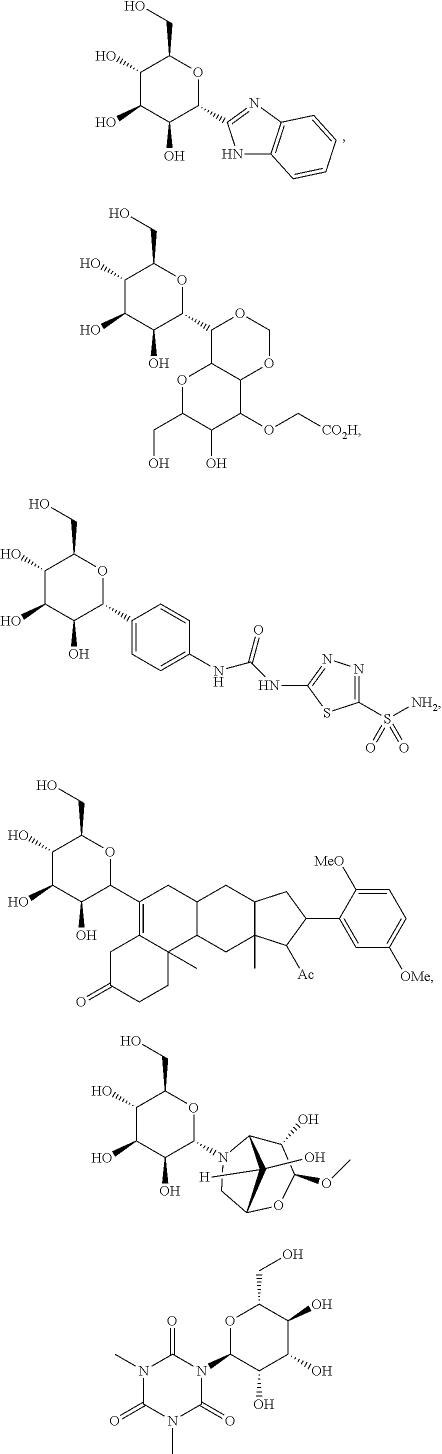

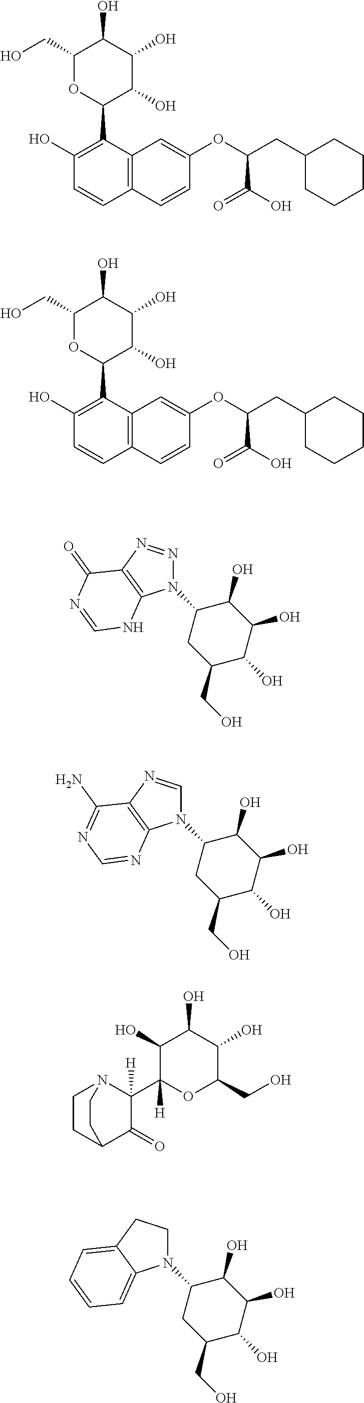 [SMILES: O=C(O)[C@H](CC1CCCCC1)Oc1ccc2ccc(O)c([C@H]3O[C@H](CO)[C@@H](O)[C@H](O)[C@@H]3O)c2c1 ; O=C(O)[C@H](CC1CCCCC1)Oc1ccc2ccc(O)c([C@H]3O[C@H](CO)[C@@H](O)[C@H](O)[C@@H]3O)c2c1 ; O=c1nc[nH]c2c1nnn2[C@H]1C[C@H](CO)[C@@H](O)[C@H](O)[C@@H]1O ; Nc1ncnc2c1ncn2[C@H]1C[C@H](CO)[C@@H](O)[C@H](O)[C@@H]1O ; O=C1C2CCN(CC2)[C@H]1[C@H]1O[C@H](CO)[C@@H](O)[C@H](O)[C@@H]1O ; OC[C@H]1C[C@H](N2CCc3ccccc32)[C@@H](O)[C@@H](O)[C@@H]1O]

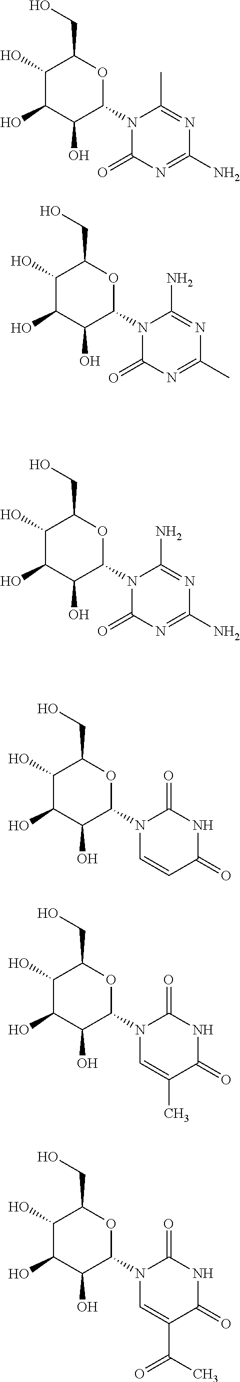 [SMILES: Cc1nc(N)nc(=O)n1[C@H]1O[C@H](CO)[C@@H](O)[C@H](O)[C@@H]1O ; Cc1nc(N)n([C@H]2O[C@H](CO)[C@@H](O)[C@H](O)[C@@H]2O)c(=O)n1 ; Nc1nc(N)n([C@H]2O[C@H](CO)[C@@H](O)[C@H](O)[C@@H]2O)c(=O)n1 ; O=c1ccn([C@H]2O[C@H](CO)[C@@H](O)[C@H](O)[C@@H]2O)c(=O)[nH]1 ; Cc1cn([C@H]2O[C@H](CO)[C@@H](O)[C@H](O)[C@@H]2O)c(=O)[nH]c1=O ; CC(=O)c1cn([C@H]2O[C@H](CO)[C@@H](O)[C@H](O)[C@@H]2O)c(=O)[nH]c1=O]

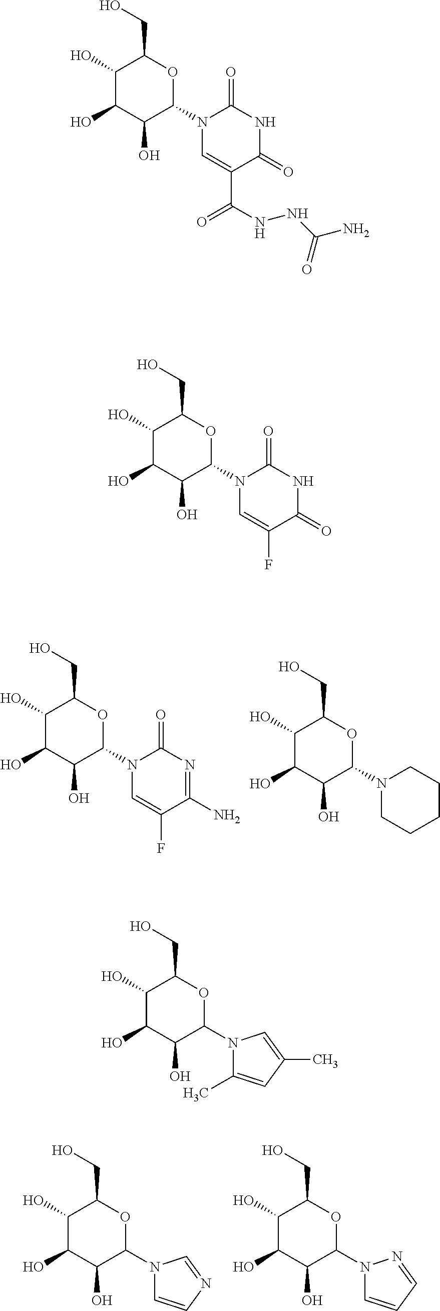

Another embodiment provides a compound of Formula (I), or a pharmaceutically acceptable salt thereof:

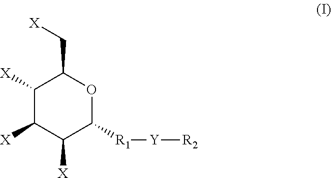

wherein:
X is —H, halogen, (C₁-C₆)alkyl, —NR₅R₆, —SR₇, or —OR₇;
Y is absent, or is —NR₈, —O—, —S—, —C(O)O—, —C(O)—, —C(O)N(R₈)(CH₂)ₘ—, —N(R₈)C(O)O—, —OC(O)NR₈—, —NR₈SO₂—, —NR₈—C(O)—, —SO₂—, —NR₈C(O)NR₈—, —S(O)—, —SO₂NR₈—, —(C₁-C₆)alkyl, or —(O—(C₁-C₆alkyl))ₙ;
R₁ is cycloalkyl, heterocyclyl, aryl, or heteroaryl; each optionally substituted with one or more R₃ groups; and
R₂ is —H, or alkyl, cycloalkyl, heterocyclyl, aryl, aralkyl, or heteroaryl; each optionally substituted with one or more R₃ groups,

wherein R₃—OH, —CN, halogen, —C(R₁₀)₃, —(CH₂)ₙOR₄, —(CH₂)ₙC(O)OR₄, —(CH₂)ₙN(R₄)₂, —C(O)OR₄, —C(O)N(R₄)₂, —N(R₄)C(O)(R₄)₂, —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂, —P(R₄)₂, —C₆H₄—R₄, or alkyl, alkenyl, alkynyl, cycloalkyl, heterocyclo, aryl, aralkyl, or heteroaryl; each optionally substituted with one or more R₄ groups,

wherein R₄ is —H, or optionally substituted C₁-C₆ alkyl, C₁-C₆ alkenyl, C₁-C₆ alkynyl, cycloalkyl, heterocyclyl, aryl or heteroaryl;

wherein R₅ and R₆ are each independently —H, optionally substituted alkyl, alkenyl, alkynyl, cycloalkyl, heterocyclyl, aryl or heteroaryl, —C(O)R₉, —C(O)NHR₉, or —C(O)OR₉;

wherein R₇ is —H, optionally substituted alkyl, alkenyl, alkynyl, cycloalkyl, heterocyclyl, heteroaryl or aryl, —C(O)R₉, or —C(O)NHR₉;

wherein R₈ is —H, optionally substituted alkyl, alkenyl, alkynyl, cycloalkyl, heterocyclyl, heteroaryl, aryl, or —C(O)R₉;

wherein R₉ is —H, optionally substituted alkyl, cycloalkyl, heterocyclyl, aryl or heteroaryl;

wherein R₁₀ is —H, —OH, halogen, or optionally substituted C₁-C₆ alkyl, C₁-C₆ alkenyl, C₁-C₆ alkynyl, cycloalkyl, heterocyclyl, aryl or heteroaryl;

wherein m is 0, 1 or 2; and

wherein n is 0, 1, 2, 3, or 4.

In some embodiments, R₁ is not indole or triazole and the compound of Formula (I) cannot have a structure selected from the group consisting of:

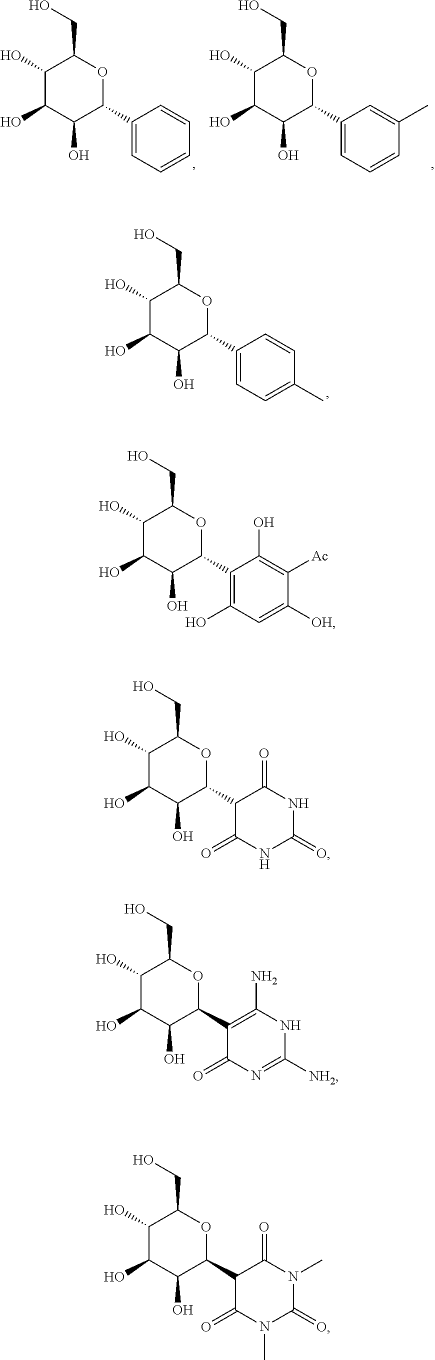

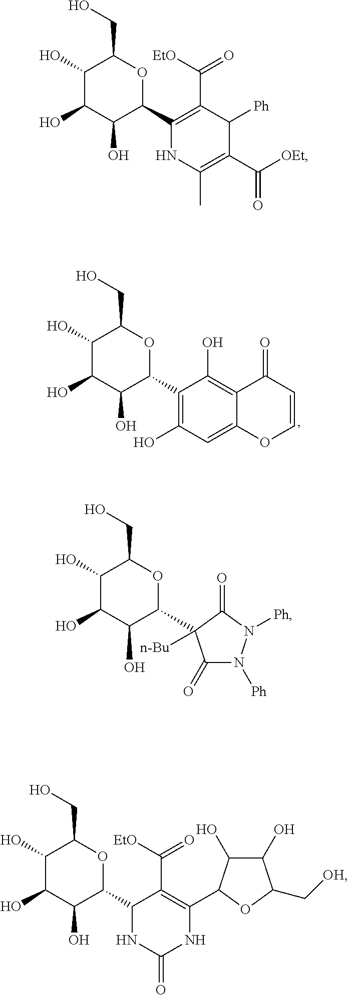

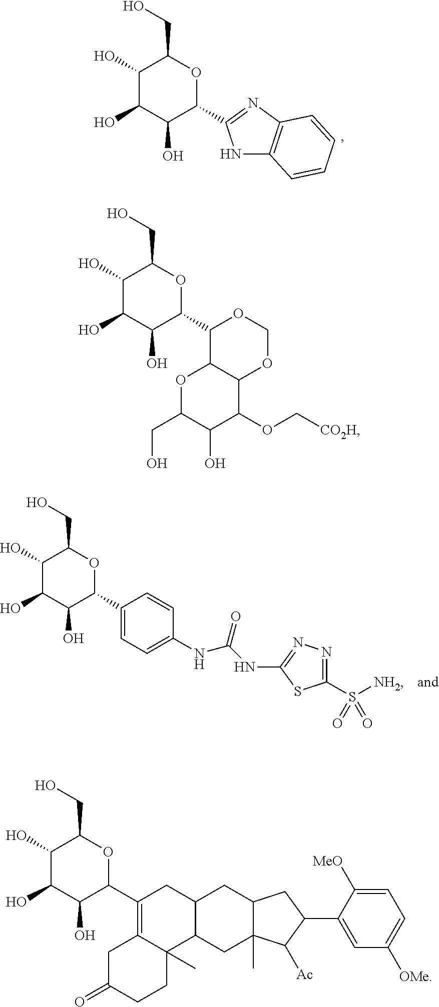 [SMILES: OC[C@H]1O[C@H](c2nc3ccccc3[nH]2)[C@@H](O)[C@@H](O)[C@@H]1O ; O=C(O)COC1C(O)C(CO)OC2C1OCOC2[C@H]1O[C@H](CO)[C@@H](O)[C@H](O)[C@@H]1O ; NS(=O)(=O)c1nnc(NC(=O)Nc2ccc([C@H]3O[C@H](CO)[C@@H](O)[C@H](O)[C@@H]3O)cc2)s1 ; COc1ccc(OC)c(C2CC3CC4CC(C5O[C@H](CO)[C@@H](O)[C@H](O)[C@@H]5O)=C5CC(=O)CCC5(C)C4CC3(C)C2C(C)=O)c1]

In some embodiments, R₁ is bonded via a carbon atom.
In other embodiments,
R₁ is cycloalkyl, heterocycle, aryl, or heteroaryl; each optionally substituted with one or more R₃ groups;
R₂ is —H, or alkyl, cycloalkyl, heterocycle, aryl, aralkyl, or heteroaryl; each optionally substituted with one or more R₃ groups,

wherein R₃ is —OH, —CN, halogen, —C(R₁₀)₃, —(CH₂)ₙOR₄, —(CH₂)ₙC(O)OR₄, —(CH₂)ₙN(R₄)₂, —C(O)OR₄, —C(O)N(R₄)₂, —N(R₄)C(O)(R₄)₂, —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂, —P(R₄)₂, —C₆H₄—R₄, or alkyl, alkenyl, alkynyl, cycloalkyl, heterocyclo, aryl, aralkyl, or heteroaryl; each optionally substituted with one or more R₄ groups,

wherein R₄ is —H, or optionally substituted C₁-C₆ alkyl, C₁-C₆ alkenyl, C₁-C₆ alkynyl, cycloalkyl, heterocycle, aryl or heteroaryl;

wherein R₅ and R₆ are each independently —H, optionally substituted alkyl, alkenyl, alkynyl, cycloalkyl, heterocycle, aryl or heteroaryl, —C(O)R₉, —C(O)NHR₉, or —C(O)OR₉;

wherein R₇ is —H, optionally substituted alkyl, alkenyl, alkynyl, cycloalkyl, heterocycle, heteroaryl or aryl, —C(O)R₉, or —C(O)NHR₉;

wherein R₈ is —H, optionally substituted alkyl, alkenyl, alkynyl, cycloalkyl, heterocycle, heteroaryl, aryl or —C(O)R₉;

wherein R₉ is —H, optionally substituted alkyl, cycloalkyl, heterocycle, aryl or heteroaryl;

wherein R₁₀ is —H, —OH, halogen, or optionally substituted C₁-C₆ alkyl, C₁-C₆ alkenyl, C₁-C₆ alkynyl, cycloalkyl, heterocycle, aryl or heteroaryl. In another embodiment of the compounds of the present invention or a pharmaceutically acceptable salt thereof, X is —OH, —F, —OCH₃, or —CH₃.

Another embodiment provides a compound of Formula (I), or a pharmaceutically acceptable salt thereof:

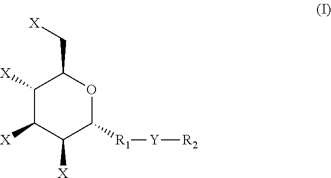

wherein
X is —OR₇;
Y is absent or a C₁-C₁₀ aliphatic wherein up to four methylene units of the C₁-C₁₀ aliphatic can be optionally replaced with —NR₈, —O—, —S—, C(O)—, —S(O)—, or —SO₂—; Y is optionally substituted with 1-2 occurrences of halogen, OH, C₃₋₆cycloalkyl or C₁₋₆aliphatic;
R₁ is C₆₋₁₀ aryl optionally substituted with one or more R_{3A} groups; and
R₂ is H, C₃-C₆ cycloalkyl, 3-8 membered heterocyclyl, C₆₋₁₀ aryl, (C₆₋₁₀ aryl)-(C₁-C₆alkyl)-, or 5-10 membered heteroaryl; each R₂ is independently and optionally substituted with one or more R_{3B} groups and optionally substituted with one R₃ group;
each R_{3A} and R_{3B} is independently halogen, —CN, NO₂, C₃-C₆ cycloalkyl, 3-8 membered heterocyclyl, (C₆₋₁₀ aryl)-(C₁-C₆alkyl)-; or a C₁-C₁₀ aliphatic wherein up to four methylene units of the C₁-C₁₀ aliphatic can be optionally replaced with —NR₄, —O—, —S—, —C(O)—, —S(O)—, —SO₂—, or —P(O)—; each R_{3A} and R_{3B} is independently and optionally substituted with one or more R₄ or R_{4A} groups;
R₃ is C₃-C₆ cycloalkyl, 3-8 membered heterocyclyl, C₆₋₁₀ aryl, (C₆₋₁₀ aryl)-(C₁-C₆alkyl)-, or 5-10 membered heteroaryl; each R₃ is optionally substituted with one or more R₄ or R_{4A} groups;
R₄ is H, C₁-C₆ alkyl, C₁-C₆ alkenyl, C₁-C₆ alkynyl, C₃₋₈ cycloalkyl, 3-8 membered heterocyclyl, C₆₋₁₀ aryl or 5-10 membered heteroaryl; each R₄ is optionally substituted with one or more R_{4B} groups;
R_{4A} is halogen, CN, NO₂, or a C₁-C₁₀ aliphatic wherein up to four methylene units of the C₁-C₁₀ aliphatic can be optionally replaced with —NR₄, —O—, —S—, —C(O)—, —S(O)—, —SO₂—, or —P(O)—; each R_{4A} is optionally substituted with 0-3 halo;
R_{4B} is halogen, CN, NO₂, or a C₁-C₁₀ aliphatic wherein up to four methylene units of the C₁-C₁₀ aliphatic can be optionally replaced with —NR, —O—, —S—, —C(O)—, —S(O)—, —SO₂—, or —P(O)—; each R_{4A} is optionally substituted with 0-3 halo;
R₇ is H or a 5-6 membered heterocyclyl having 1-2 heteroatoms selected from oxygen, nitrogen, or sulfur; wherein said 5-6 membered heterocyclyl is independently and optionally substituted with 1-4 occurrences of C₁₋₄alkyl wherein up to one methylene unit of the C₁₋₄alkyl is optionally replaced with —O—;
R₈ is H, C₁-C₆ alkyl, C₁-C₆ alkenyl, C₁-C₆ alkynyl, C₃-C₆ cycloalkyl, 3-8 membered heterocyclyl, C₆₋₁₀ aryl or 5-10 membered heteroaryl; or —C(O)R₉;
R₉ and R₁₀ are each independently C₁-C₆ alkyl or C₃-C₆ cycloalkyl;
R is H, C₁-C₆ alkyl or C₃-C₆ cycloalkyl;
m is 0, 1 or 2; and
n is 0, 1, 2, 3, or 4.
   In some embodiments, the compound is not one of the following:

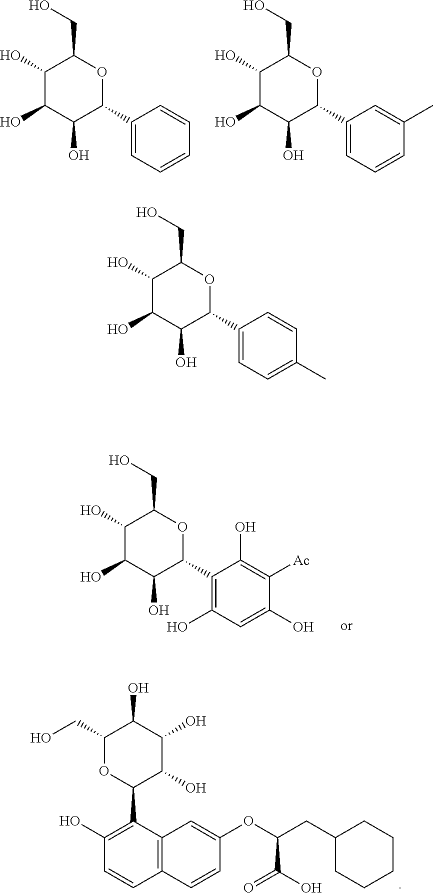

In some embodiments, R₁ is optionally substituted with 1-4 R_{3A} groups; or in some embodiments, 1-2 R_{3A} groups. In other embodiments, R₂ is optionally substituted with 1-4 R_{3B} groups; or in some embodiments, 1-2 R_{3B} groups. In some embodiments, each R_{3A} and R_{3B} optionally substituted with 1-4 R_{4A} groups; or in some embodiments, 1-2 R_{4A} groups. In other embodiments, R₂ is optionally substituted with one R₃ group.

In some embodiments, R₃ is optionally substituted with 1-4 R₄ or R_{4A} groups; or in some embodiments, 1-2 R₄ or R_{4A} groups. In some embodiments, R₄ is optionally substituted with 1-4 R_{4B} groups; or in some embodiments, 1-2 R_{4B} groups.

According to another embodiment,
Y is absent, or is —NR₈, —O—, —S—, —C(O)—, —C(R¹⁰)(OH)—, —C(O)N(R₈)(CH₂)ₘ—, —N(R₈)C(O)O—, —OC(O)NR₈—, —NR₈SO₂—, —NR₈—C(O)—, —SO₂—, —NR₈C(O)NR₈—, —S(O)—, —SO₂NR₈, —(C₁-C₆)alkyl-, —(C₁-C₆)alkenyl-, —(C₁-C₆)alkynyl-, —(O—(C₁-C₆ alkyl))ₙ-, —O—(C₁₋₆alkyl)NR₈C(O)—, —O—(C₁₋₆alkyl)C(O)NR₈, —O—(C₁₋₆alkyl)-C(O)—, or —((C₁-C₆)alkyl)-O—;
each R_{3A} and R_{3B} is independently —OH, —CN, halogen, —C(R₁₀)₃, —C(R₁₀)₂OH, —(CH₂)ₙOR₄, —(CH₂)ₙC(O)OR₄, —(CH₂)ₙN(R₄)₂, —C(O)OR₄, —C(O)N(R₄)₂, —N(R₄)C(O)(R₄)₂, —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, or —P(O)(R₄)₂; and
R_{4A} is —OH, —CN, halogen, —C(R₁₀)₃, —C(R₁₀)₂OH, —(CH₂)ₙOR₄, —(CH₂)ₙC(O)OR₄, —(CH₂)ₙN(R₄)₂, —C(O)OR₄, —C(O)N(R₄)₂, —N(R₄)C(O)(R₄)₂, —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂;
R⁷ is H or mannosyl.
   According to another embodiment,
   X is —OH;
   Y is absent, or is —NR₈, —O—, —S—, —C(O)—, —C(R¹⁰)(OH)—, —SO₂—, —S(O)—, —(C₁-C₆)alkyl, —(C₁-C₆)alkenyl, —(C₁-C₆)alkynyl, —(O—(C₁-C₆ alkyl))ₙ-, —O(C₁₋₆alkyl)N—R₈C(O)—, —O—(C₁₋₆alkyl)-C(O)NR₈, —O—(C₁₋₆alkyl)C(O)—, or —((C₁-C₆)alkyl)-O—;
   R₂ is C₆₋₁₀ aryl, (C₆₋₁₀ aryl)-(C₁-C₆alkyl)-, or 5-10 membered heteroaryl; each R₂ is independently and optionally substituted with one or more R_{3B} and optionally one R₃;
   R₃ is C₆₋₁₀ aryl, (C₆₋₁₀ aryl)-(C₁-C₆alkyl)-, or 5-10 membered heteroaryl; each R₃ is independently and optionally substituted with one or more groups selected from R₄ or R_{4A}; and
   R₈ is —H, C₁-C₆ alkyl, C₁-C₆ alkenyl, C₁-C₆ alkynyl, or C₃-C₆ cycloalkyl.
      In another embodiment of the compounds of the present invention or a pharmaceutically acceptable salt thereof,
   X is —OH;
   Y is absent, or is —O—, —S—, —OC(O)NR₈—, or —C(O)N(R₄)(CH₂)ₘ—;
   R₁ is aryl optionally substituted with one or more R_{3A} and groups; and
   R₂ is —H, or alkyl, cycloalkyl, heterocycle, aryl, aralkyl, or heteroaryl; each optionally substituted with one or more R_{3B} groups and optionally one R₃;
   each R_{3A} and R_{3B} is independently —OH, —CN, halogen, —C(R₁₀)₃, —(CH₂)ₙOR₄, —(CH₂)ₙC(O)OR₄, —(CH₂)ₙN(R₄)₂, —C(O)R₄, —C(O)N(R₄)₂, —N(R₄)C(O)(R₄)₂, —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, C₁-C₆ alkyl, aryl, aralkyl, or heteroaryl; each optionally substituted with one or more R₄ groups;
   wherein each R₄ is independently —H or C₁-C₆ alkyl;
   wherein m is 0, 1 or 2; and
   wherein n is 0, 1, or 2.
      According to another embodiment

X is —OH;

Y is absent;

R₁ is phenyl optionally substituted with one or more halogen, —OR₄, or —(CH₂)ₙC(O)OR₄;

R₂ is heteroaryl optionally substituted with one or more R_{3B} groups;

R_{3B} is C₁-C₆ alkyl or C(R₁₀)₃; and

R₄ is H or C₁-C₆ alkyl.

According to another embodiment

X is —OH;

Y is absent;

R₁ is phenyl optionally substituted with one or more halogen, —OR₄, or —(CH₂)ₙC(O)OR₄;

R₂ is aryl optionally substituted with one or more R_{3B} groups;

R_{3B} is —OH, halogen, —CN, —OR₄, —(CH₂)ₙC(O)OR₄, —(CH₂)ₙOR₄, —(CH₂)₆N(R₄)₂, —C(O)NHR₄, —NH—C(O)R₄, —SO₂R₄, or —C(O)OR₄; and

R₄ is H or C₁-C₆ alkyl.

In another embodiment of the compounds of the present invention or a pharmaceutically acceptable salt thereof, Y is —C(O)N(R₄)(CH₂)ₘ—, particularly —C(O)NH—.

In another embodiment of the compounds of the present invention or a pharmaceutically acceptable salt thereof, Y is —OC(O)NR₈—, particularly —OC(O)NH—.

In another embodiment of the compounds of the present invention or a pharmaceutically acceptable salt thereof, Y is C₁-C₆ alkyl, C₁-C₆ alkenyl, or C₁-C₆ alkynyl.

In another embodiment of the compounds of the present invention or a pharmaceutically acceptable salt thereof, Y is absent.

In another embodiment of the compounds of the present invention or a pharmaceutically acceptable salt thereof, Y is —O—.

In another embodiment of the compounds of the present invention or a pharmaceutically acceptable salt thereof, R₁ is optionally substituted phenyl. In other embodiments, R₁ is optionally substituted naphthyl.

In another embodiment of the compounds of the present invention or a pharmaceutically acceptable salt thereof, R₁ is optionally substituted phenyl, particularly R₁ is phenyl substituted with one or more halogen, —OR₄, or —(CH₂)ₙC(O)OR₄. In some embodiments, R₁ is phenyl substituted with one or more halogen, —(C₁-C₆alkyl), or C₁-C₆alkyl. In other embodiments, R₁ is phenyl substituted with one or more R_{3A}, wherein R_{3A} is halogen, C₁-C₆alkyl, C₁-C₆alkenyl, C₁-C₆alknyl, or a C₁-C₁₀ aliphatic wherein up to four methylene units of the C₁-C₁₀ aliphatic can be optionally replaced with —NR₄, —O—, or —C(O)—. In some embodiments, R₄ is —H or C₁-C₆ alkyl.

In yet another embodiment, R₁ is phenyl substituted with one or more R_{3A}, wherein R_{3A} is fluoro, bromo, chloro, CH₃, CH₂CH₃, —C≡CH, OH, OCH₃, OCF₃, —OCH₂C(CH₃)₃, —O(CH₂)₄CF₃, —OCH₂C(O)NHCH₃, —OCH₂C(O)OCH₃, —OCH₂C≡CCH₂CH₃, —O(CH₂)₃CN, —OCH₂CH(CH₃)CH₂CH₃, —OCH₂CH₂CH(CH₃)₂, —O(CH₂)₃OCH₃, —O(CH₂)₂F, —O(CH₂)₃F, or —CH₂CH₂C(O)OCH₃.

In another embodiment of the compounds of the present invention or a pharmaceutically acceptable salt thereof, R₂ is a heteroaryl ring optionally substituted with one or more R_{3B} groups and optionally one R₃. In one example the heteroaryl ring is selected from the group consisting of: pyrazole, thiadiazole, quinoline, indole, thiazole, pyridine and benzothiazile; in another example the heteroaryl ring is selected from the group consisting of: pyrimidine, benzodioxole, benzodioxane, benzothiophene, indole, pyrazole and benzimidazole. In another embodiment, the heteroaryl ring is selected from the group consisting of: imidazolyl, pyrazolyl, triazolyl, thienyl, thiadiazolyl, thiazolyl, oxadiazolyl, pyridinyl, pyrimidinyl, benzodioxolyl, indolyl, benzimidazolyl, benzothiazolyl, benzooxadiazolyl, imidazopyridinyl, quinolinyl, oxetanyl, tetrahydropyranyl, and C₃₋₆cycloalkyl.

In another embodiment of the compounds of the present invention or a pharmaceutically acceptable salt thereof, R₂ is aryl optionally substituted with one or more R_{3B} groups and optionally one R₃, particularly R₂ is phenyl or naphthalene each optionally substituted with one or more R_{3B} groups and optionally one R₃.

In some embodiments, R₂ is substituted with one R₃ group. In some embodiments, R₃ is phenyl as shown in the formula below.

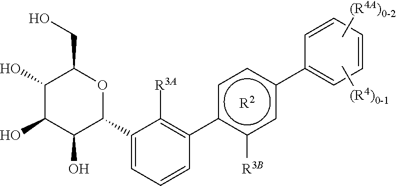

In some embodiments, R^{3A}, R^{3B}, and R^{4A} are each independently halogen, —O(C₁-C₆alkyl), or C₁-C₆alkyl and R² is a 6-membered aryl or heteroaryl ring.

In some embodiments, R_{3B} is halogen, CN, NO₂, or a C₁₋₆ aliphatic wherein up to four methylene units of the C₁₋₆ aliphatic can be optionally replaced with —NR₄, —O—, —C(O)— or —S(O)₂—, wherein R_{3B} is optionally substituted with one or more halogen. In some embodiments, R_{3B} is independently halogen, —O(C₁-C₆alkyl), or C₁-C₆alkyl. In other embodiments, R_{3B} is fluoro, chloro, CN, NO₂, NH₂, CH₃, CF₃, C(O)CH₃, C(O)NH(CH₃), CH₂OH, OH, butyl, CH₂C(O)NHCH₃, or S(O)₂CH₃. In yet other embodiments, R_{3B} is C₁-C₆ alkyl, or —C(R₁₀)₃. In some embodiments, R_{3B} is fluoro, chloro, CN, CH₃, CH₂CH₃, CH₂CH₂CH₃, C(CH₃)₃, C(O)CH₃, CH₂C(O)OCH₃, C(O)OH, C(O)OCH₃, C(O)NHCH₃, NHC(O)CH₃, NHC(O)CHC(CH₃)₂, CH₂OH, CH₂OCH₃, CH₂N(CH₃)₂, NH₂, N(CH₃)₂, OH, —OCH₃, O(CH₂)₂CH₃, S(O)₂NHCH₃, or S(O)₂CH₃. In other embodiments, R_{3B} is —OH, halogen, —CN, —OR₄, —(CH₂)ₙC(O)OR₄, —(CH₂)ₙOR₄, —(CH₂)₆N(R₄)₂, —C(O)NHR₄, —NH—C(O)R₄, —SO₂R₄, or —C(O)OR₄.

In another embodiment of the compounds of the present invention or a pharmaceutically acceptable salt thereof, R₂ is C₁-C₆ alkyl, cycloalkyl, or araryl optionally substituted with one or more R_{3B} groups. In some embodiments, R₂ is also substituted with one R₃ group.

In another embodiment of the compounds of the present invention or a pharmaceutically acceptable salt thereof, R₂ is aryl, araryl or heteroaryl optionally substituted with one or more R_{3B} groups, particularly R₂ is phenyl, benzyl, or thiophenyl each optionally substituted with one or more R_{3B} groups. In some embodiments, R₂ is also substituted with one R₃ group.

According to another embodiment, R₁ is phenyl and R₂ is phenyl.

Another embodiment provides a compound as represented by the following formula:

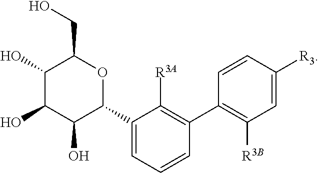

In some embodiments, each R^{3A} and R^{3B} is independently halogen, C₁-C₆alkyl, —O(C₁-C₆alkyl); and R₃ is a heteroaryl ring optionally substituted with one or more R₄ or R_{4A} group. In some embodiments, R₃ is a 5-membered heteroaryl, particularly an oxadiazolyl, pyrazolyl, or thiadiazolyl. In some embodiments R₃ is a heteroaryl ring selected from oxadiazolyl.

In another embodiment of the compounds of the present invention or a pharmaceutically acceptable salt thereof, R₂ is —H.

In another embodiment of the compounds of the present invention or a pharmaceutically acceptable salt thereof, each R^{3A} and R^{3B} is independently halogen, C₁-C₆ alkyl, or benzyl.

In another embodiment of the compounds of the present invention or a pharmaceutically acceptable salt thereof, each R^{3A} and R^{3B} is independently halogen, C₁-C₆ alkyl, or —N(R₄)₂.

In another embodiment of the compounds of the present invention or a pharmaceutically acceptable salt thereof, each R^{3A} and R^{3B} is independently is C₁-C₆ alkyl, or —C(R₁₀)₃.

In another embodiment of the compounds of the present invention or a pharmaceutically acceptable salt thereof, each R^{3A} and R^{3B} is independently is halogen, C₁-C₆alkyl, or —O(C₁-C₆alkyl).

In another embodiment of the compounds of the present invention or a pharmaceutically acceptable salt thereof, each R^{3A} and R^{3B} is independently —OH, halogen, —CN, —OR₄, —(CH₂)ₙC(O)OR₄, —(CH₂)ₙOR₄, —(CH₂)ₙN(R₄)₂, —C(O)NHR₄, —NH—C(O)R₄, —SO₂R₄, or —C(O)OR₄.

In another embodiment of the compounds of the present invention or a pharmaceutically acceptable salt thereof each R^{3A} and R^{3B} is independently halogen, C₁-C₆ alkyl, —(CH₂)ₙC(O)OR₄, or —C(O)NHR₄. In other embodiments, each R^{3A} and R^{3B} is independently is halogen, C₁-C₆alkyl, —O(C₁-C₆alkyl).

In another embodiment of the compounds of the present invention or a pharmaceutically acceptable salt thereof, R₃ is a heteroaryl ring optionally substituted with one or more R₄ or R_{4A} groups, particularly the heteroaryl ring is oxadiazole. In some embodiments, R₃ is a heteroaryl ring optionally substituted with one or more R₄ groups.

In another embodiment of the compounds of the present invention or a pharmaceutically acceptable salt thereof, R₄ is —H or C₁-C₆ alkyl.

According to another embodiment, X is —OR₇ and R₇ is H or

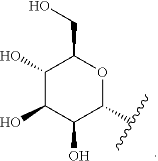 [SMILES: *[C@H]1O[C@H](CO)[C@@H](O)[C@H](O)[C@@H]1O]

In some embodiments, R₇ is bonded as shown in Formula IA, IB, IC, or ID:

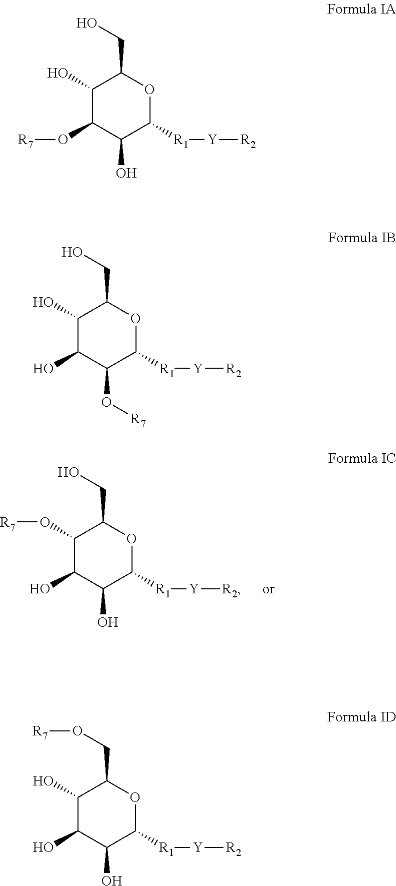

wherein R₁, Y, and R₂ are as defined in any one of claims.
In other embodiments, R₇ is H. In some embodiments, X is —OH, —F, —OCH₃, or —CH₃.
According to another embodiment,

### X is —OR₇ and R₇ is H or

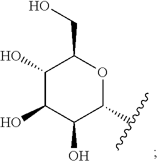 [SMILES: *[C@H]1O[C@H](CO)[C@@H](O)[C@H](O)[C@@H]1O]

R₁ is phenyl or naphthyl;
Y is absent, or is —O—, —C(O)N(R₈)(CH₂)ₘ—, —OC(O)NR₈—, —(C₁-C₆)alkyl-, —(C₁-C₆)alkenyl-, —(C₁-C₆)alkynyl-, —(O—(C₁-C₆ alkyl))ₙ—, —O(C₁₋₆alkyl)NR₈C(O)—, —O(C₁₋₆alkyl)C(O)NR₈, —O(C₁₋₆alkyl)C(O)—, or —((C₁-C₆)alkyl)-O—;
R₂ is C₆₋₁₀aryl, a 5-6 membered monocyclic heteroaryl having 1-4 heteroatoms selected from oxygen, nitrogen, or sulfur; or an 8-10 membered bicyclic heteroaryl having 1-4 heteroatoms selected from oxygen, nitrogen, or sulfur; a 3-8 membered monocyclic heterocyclyl having 1-2 heteroatoms selected from oxygen, nitrogen, or sulfur; or a C₃₋₆cycloalkyl; and
R³ is phenyl or a 5-6 membered monocyclic heteroaryl having 1-4 heteroatoms selected from oxygen, nitrogen, or sulfur.
According to yet another embodiment,
X is —OR₇ and R₇ is H or

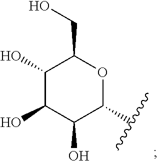 [SMILES: *[C@H]1O[C@H](CO)[C@@H](O)[C@H](O)[C@@H]1O]

R₁ is phenyl or naphthyl;
Y is absent, or is —O—, —C(O)N(R₈)(CH₂)ₘ—, —OC(O)NR₈—, —(C₁-C₆)alkyl-, —(C₁-C₆)alkenyl-, —(C₁-C₆)alkynyl-, —(O—(C₁-C₆alkyl)ₙ-, —O(C₁₋₆alkyl)NR₈C(O)—, —O(C₁₋₆alkyl)C(O)NR₈, —O(C₁₋₆alkyl)C(O)—, or —((C₁-C₆)alkyl)-O—;
R₂ is phenyl, naphthyl, imidazolyl, pyrazolyl, triazolyl, thienyl, thiadiazolyl, thiazolyl, oxadiazolyl, pyridinyl, pyrimidinyl, benzodioxolyl, indolyl, benzimidazolyl, benzothiazolyl, benzooxadiazolyl, imidazopyridinyl, quinolinyl, oxetanyl, tetrahydropyranyl, and C₃₋₆cycloalkyl; and
R³ is phenyl, pyrazolyl, triazolyl, oxazolyl, isoxazolyl, oxadiazolyl, thiazolyl, thiadiazolyl, pyridinyl.

In some embodiments, Y is absent or is —O—, —S—, —C(O)O—, —C(O)—, —C(O)N(R₄)—, —N(R₄)C(O)O—, —OC(O)NR₄—, —NR₄SO₂—, —NR₄—, —NR₄—C(O)—, —SO₂—, —NR₄C(O)NR₄—, —S(O)—, —SO₂NR₄—, —(O—(C₁-C₆alkyl))ₙ, or optionally substituted alkyl, alkenyl, alkynyl, cycloalkyl, heterocycle, aryl or heteroaryl;

In some embodiments, R₄ is independently —H, or optionally substituted C₁-C₆ alkyl, cycloalkyl, heterocycle, aryl or heteroaryl;

In some embodiments, R₅ and R₆ are each independently —H, optionally substituted alkyl, alkenyl, alkynyl, cycloalkyl, heterocycle, aryl or heteroaryl, —C(O)R₉, —C(O)NHR₉, —C(O)OR₉, C(O)NR₉SO₂—R₉ or S(O)₂R₉; and R₇ is —H, optionally substituted alkyl, alkenyl, alkynyl, cycloalkyl, heterocycle, heteroaryl, aryl, —C(O)R₉, or —C(O)NHR₉.

Without being bound by theory, certain compounds of the present invention can be metabolized by mannosides into compounds that are active as FimH inhibitors. Such compounds have Formula (I), or a pharmaceutically acceptable salt thereof:

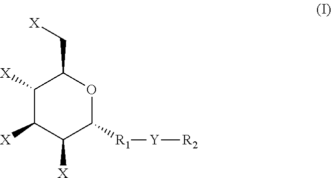

wherein X is —OR₇ and R₇ is a sugar derivative, such as a mannose derivative.

In some embodiments, the compound is represented by one of the following formulae:

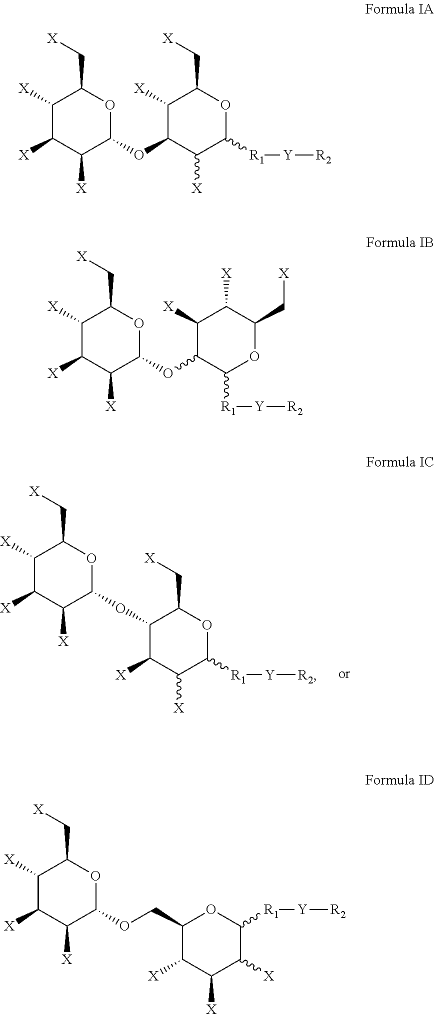

or a pharmaceutically acceptable salt thereof, wherein X, R₁, Y, and R₂ are as defined herein. In some embodiments, X is OH.

In some embodiments.
X is —H, halogen, (C₁-C₆)alkyl, —NR₅R₆, —SR₇, or —OR₇;
Y is absent or is —O—, —S—, —C(O)O—, —C(O)—, —C(O)N(R₄)—,
—N(R₄)C(O)O—, —OC(O)NR₄—, —NR₄SO₂—, NR₄—, —NR₄—C(O)—, —SO₂—,
—NR₄C(O)NR₄—, —S(O)—, —SO₂NR₄—, —(O—(C₁-C₆ alkyl))ₙ, or optionally substituted alkyl, alkenyl, alkynyl, cycloalkyl, heterocycle, aryl or heteroaryl;
R₁ is alkyl, alkenyl, alkynyl, cycloalkyl, heterocycle, aryl or heteroaryl; each optionally substituted with one or more R₃ groups,
R₂ is —H, or alkyl, cycloalkyl, heterocycle, aryl or heteroaryl; each optionally substituted with one or more R₃ groups,
wherein R₃ is halogen, —OR₄, —C(O)OR₄, —C(O)R₄, —C(O)N(R₄)₂, —OC(O)N(R₄)₂, —NR₄C(O)OR₄, —NR₄SO₂R₄, —N(R₄)₂, —NR₄C(O)R₄, —SO₂—R₄, —NR₄C(O)N(R₄)₂, —S(O)R₄, —SO₂N(R₄)₂, —SR₄, —P(O)(OR₄)₂, —P(O)(R₄)², —P(R₄)₂, —C₆H₄—R₄, or alkyl, alkenyl, alkynyl, cycloalkyl, heteroaryl or heterocyclo; each optionally substituted with one or more R₄ groups,
wherein each R₄ is independently —H, or optionally substituted C₁-C₆ alkyl, cycloalkyl, heterocycle, aryl or heteroaryl;
wherein R₅ and R₆ are each independently —H, optionally substituted alkyl, cycloalkyl, heterocycle, aryl or heteroaryl, —C(O)R₈, —C(O)NR₈SO₂—R₈, —S(O)₂R₈, or —C(O)OR₈;
wherein R₇ is —H, optionally substituted alkyl, cycloalkyl, heterocycle, heteroaryl or aryl, —C(O)R₈, or —C(O)NR₅R₆;
wherein R₈ is —H, optionally substituted alkyl, cycloalkyl, heterocycle, aryl or heteroaryl; and
wherein n is 0, 1, 2, 3, or 4.

In another embodiment the compounds of the present invention are represented by the following structural formula:

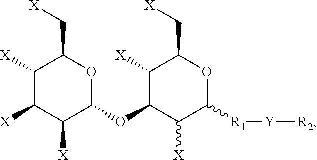

or a pharmaceutically acceptable salt thereof.

In another embodiment the compounds of the present invention are represented by the following structural formula:

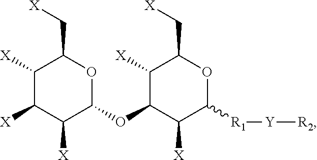

or a pharmaceutically acceptable salt thereof.

In another embodiment of the compound or a pharmaceutically acceptable salt thereof, X is —OH, —F, —OCH₃, or —CH₃.

In another embodiment the compounds of the present invention are represented by the following structural formula:

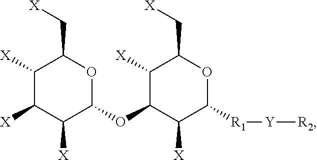

or a pharmaceutically acceptable salt thereof, wherein:
X is —OH;
Y is absent, or is —O— or —S—; and
R₁ is alkyl, alkenyl, or aryl; each optionally substituted with one or more R₃ groups;
R₂ is —H, or alkyl, cycloalkyl, heterocycle, aryl or heteroaryl; each optionally substituted with one or more R₃ groups;
wherein R₃ is —OH, halogen, —C(O)NHR₄, —NHC(O)R₄, —OR₄, —C(O)OR₄, —SO₂—R₄, or alkyl, cycloalkyl, or heterocycle optionally substituted with one or more R₄ group; each R₄ is independently —H or optionally substituted C₁-C₆ alkyl.

In another embodiment of the compound or a pharmaceutically acceptable salt thereof, Y is —O—.

In another embodiment of the compound or a pharmaceutically acceptable salt thereof, Y is absent.

In another embodiment of the compound or a pharmaceutically acceptable salt thereof, R₁ is optionally substituted phenyl.

In another embodiment of the compound or a pharmaceutically acceptable salt thereof, R₁ is hydroxyl substituted phenyl.

In another embodiment of the compound or a pharmaceutically acceptable salt thereof, R₁ is methoxy substituted phenyl.

In another embodiment of the compound or a pharmaceutically acceptable salt thereof, R₁ is alkenyl, in particular propenyl.

In another embodiment of the compound or a pharmaceutically acceptable salt thereof, R₁ is C1-C6 alkyl, in particular propyl.

In another embodiment of the compound or a pharmaceutically acceptable salt thereof, R₂ is —H.

In another embodiment of the compound or a pharmaceutically acceptable salt thereof, R₂ is optionally substituted C₁-C₆ alkyl.

In another embodiment of the compound or a pharmaceutically acceptable salt thereof, R₂ is unsubstituted C₁-C₆ alkyl, in particular methyl.

In another embodiment of the compound or a pharmaceutically acceptable salt thereof, R₂ is substituted C₁-C₆ alkyl substituted with halogen, in particular with —F.

In another embodiment of the compound or a pharmaceutically acceptable salt thereof, R₂ is cycloalkyl substituted C₁-C₆ alkyl.

In another embodiment of the compound or a pharmaceutically acceptable salt thereof, R₂ is cyclopentyl substituted C₁-C₆ alkyl.

In another embodiment of the compound or a pharmaceutically acceptable salt thereof, R₂ is —C(O)O—CH₃ substituted C₁-C₆ alkyl.

In another embodiment of the compound or a pharmaceutically acceptable salt thereof, R₂ is optionally substituted phenyl.

In another embodiment of the compound or a pharmaceutically acceptable salt thereof, R₂ is phenyl substituted with halogen, in particular with —F.

In another embodiment of the compound or a pharmaceutically acceptable salt thereof, R₂ is phenyl substituted with one or more C₁-C₆ alkyl, in particular methyl.

In another embodiment of the compound or a pharmaceutically acceptable salt thereof, R₂ is phenyl substituted with one or more amide, in particular with —C(O)NHR₄, wherein R₄ is C₁-C₆ alkyl and preferably methyl.

In another embodiment of the compound or a pharmaceutically acceptable salt thereof, R₂ is phenyl substituted with one or more —NHC(O)R₄, wherein R₄ is C₁-C₆ alkyl and preferably isopropyl.

In another embodiment of the compound or a pharmaceutically acceptable salt thereof, R₂ is oxadiazole substituted phenyl, and the oxadiazole is further substituted with C1-C6 alkyl, in particular the oxadiazole is methyl substituted 1,3,4-oxadiazole.

In another embodiment of the compound or a pharmaceutically acceptable salt thereof, R₂ is phenyl substituted with —SO₂—(C₁-C₆)alkyl, in particular with —SO₂—CH₃.

In another embodiment of the compound or a pharmaceutically acceptable salt thereof, R₂ is a diazine, in particular a pyrimidine.

In another embodiment of the compound or a pharmaceutically acceptable salt thereof, R₂ is a cycloalkyl, in particular a cyclohexane.

In another embodiment of the compound or a pharmaceutically acceptable salt thereof, R₂ is benzimidazole substituted with C₁-C₆ alkyl, in particular methyl.

In another embodiment of the compound or a pharmaceutically acceptable salt thereof, R₂ is phenyl substituted with —C(O)O—(C₁-C₆)alkyl, in particular with —C(O)O—CH₃. Another embodiment provides a compound as described in Table 1:

TABLE 1

| | |
|---|---|
| | 1 |
| 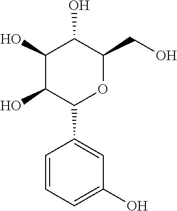 | |
| 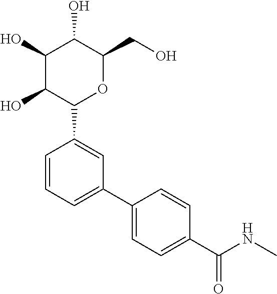 | 2 |
| 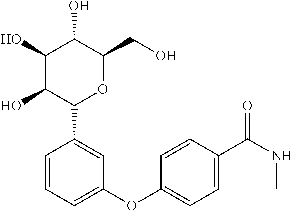 | 3 |
| 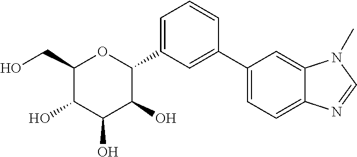 [SMILES: Cn1cnc2ccc(-c3cccc([C@H]4O[C@H](CO)[C@@H](O)[C@H](O)[C@@H]4O)c3)cc21] | 4 |
| 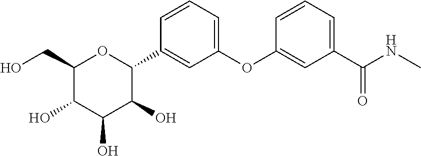 [SMILES: CNC(=O)c1cccc(Oc2cccc([C@H]3O[C@H](CO)[C@@H](O)[C@H](O)[C@@H]3O)c2)c1] | 5 |
| 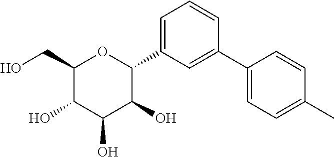 [SMILES: Cc1ccc(-c2cccc([C@H]3O[C@H](CO)[C@@H](O)[C@H](O)[C@@H]3O)c2)cc1] | 6 |
| 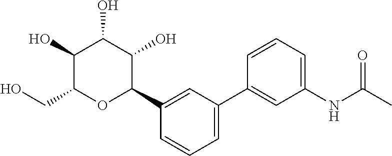 [SMILES: CC(=O)Nc1cccc(-c2cccc([C@H]3O[C@H](CO)[C@@H](O)[C@H](O)[C@@H]3O)c2)c1] | 7 |
| 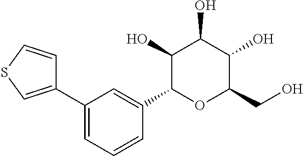 [SMILES: OC[C@H]1O[C@H](c2cccc(-c3ccsc3)c2)[C@@H](O)[C@@H](O)[C@@H]1O] | 8 |
| 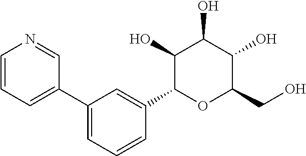 [SMILES: OC[C@H]1O[C@H](c2cccc(-c3cccnc3)c2)[C@@H](O)[C@@H](O)[C@@H]1O] | 9 |
| 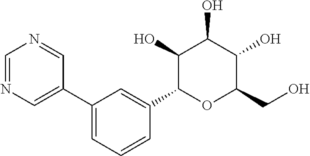 [SMILES: OC[C@H]1O[C@H](c2cccc(-c3cncnc3)c2)[C@@H](O)[C@@H](O)[C@@H]1O] | 10 |
| 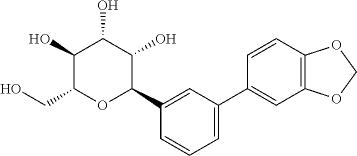 [SMILES: OC[C@H]1O[C@H](c2cccc(-c3ccc4c(c3)OCO4)c2)[C@@H](O)[C@@H](O)[C@@H]1O] | 11 |
| 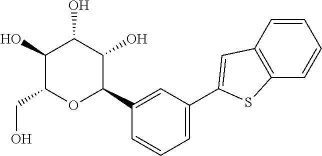 [SMILES: OC[C@H]1O[C@H](c2cccc(-c3cc4ccccc4s3)c2)[C@@H](O)[C@@H](O)[C@@H]1O] | 12 |
| 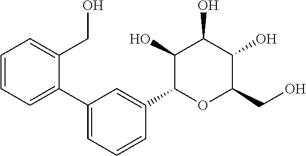 [SMILES: OCc1ccccc1-c1cccc([C@H]2O[C@H](CO)[C@@H](O)[C@H](O)[C@@H]2O)c1] | 13 |
| 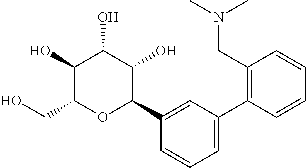 [SMILES: CN(C)Cc1ccccc1-c1cccc([C@H]2O[C@H](CO)[C@@H](O)[C@H](O)[C@@H]2O)c1] | 14 |
| 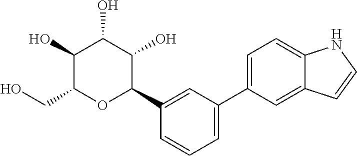 [SMILES: OC[C@H]1O[C@H](c2cccc(-c3ccc4[nH]ccc4c3)c2)[C@@H](O)[C@@H](O)[C@@H]1O] | 15 |
| 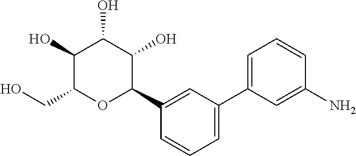 [SMILES: Nc1cccc(-c2cccc([C@H]3O[C@H](CO)[C@@H](O)[C@H](O)[C@@H]3O)c2)c1] | 16 |
| 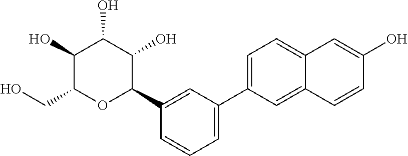 [SMILES: OC[C@H]1O[C@H](c2cccc(-c3ccc4cc(O)ccc4c3)c2)[C@@H](O)[C@@H](O)[C@@H]1O] | 17 |
| 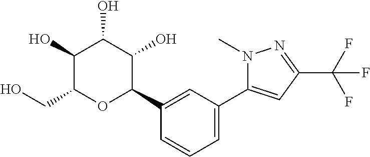 [SMILES: Cn1nc(C(F)(F)F)cc1-c1cccc([C@H]2O[C@H](CO)[C@@H](O)[C@H](O)[C@@H]2O)c1] | 18 |
| 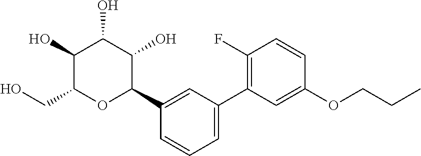 [SMILES: CCCOc1ccc(F)c(-c2cccc([C@H]3O[C@H](CO)[C@@H](O)[C@H](O)[C@@H]3O)c2)c1] | 19 |
| 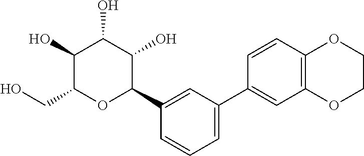 [SMILES: OC[C@H]1O[C@H](c2cccc(-c3ccc4c(c3)OCCO4)c2)[C@@H](O)[C@@H](O)[C@@H]1O] | 20 |
| 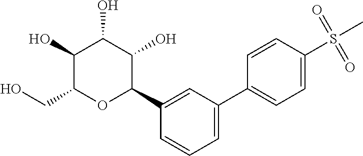 [SMILES: CS(=O)(=O)c1ccc(-c2cccc([C@H]3O[C@H](CO)[C@@H](O)[C@H](O)[C@@H]3O)c2)cc1] | 21 |
| 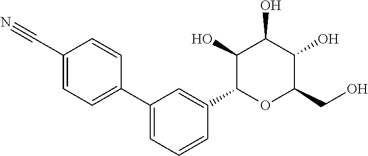 [SMILES: N#Cc1ccc(-c2cccc([C@H]3O[C@H](CO)[C@@H](O)[C@H](O)[C@@H]3O)c2)cc1] | 22 |
| 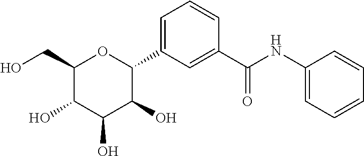 [SMILES: O=C(Nc1ccccc1)c1cccc([C@H]2O[C@H](CO)[C@@H](O)[C@H](O)[C@@H]2O)c1] | 23 |
| 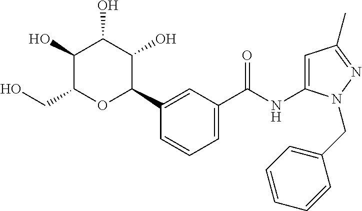 [SMILES: Cc1cc(NC(=O)c2cccc([C@H]3O[C@H](CO)[C@@H](O)[C@H](O)[C@@H]3O)c2)n(Cc2ccccc2)n1] | 24 |
| 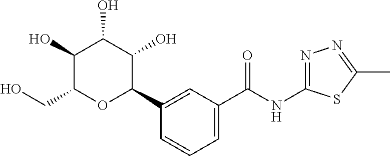 [SMILES: Cc1nnc(NC(=O)c2cccc([C@H]3O[C@H](CO)[C@@H](O)[C@H](O)[C@@H]3O)c2)s1] | 25 |
| 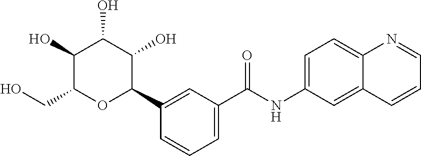 [SMILES: O=C(Nc1ccc2ncccc2c1)c1cccc([C@H]2O[C@H](CO)[C@@H](O)[C@H](O)[C@@H]2O)c1] | 26 |
| 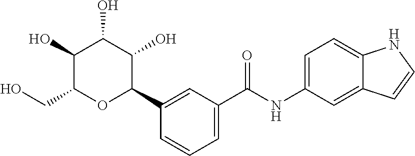 [SMILES: O=C(Nc1ccc2[nH]ccc2c1)c1cccc([C@H]2O[C@H](CO)[C@@H](O)[C@H](O)[C@@H]2O)c1] | 27 |
| 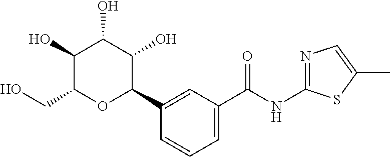 [SMILES: Cc1cnc(NC(=O)c2cccc([C@H]3O[C@H](CO)[C@@H](O)[C@H](O)[C@@H]3O)c2)s1] | 28 |
| 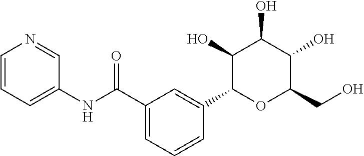 [SMILES: O=C(Nc1cccnc1)c1cccc([C@H]2O[C@H](CO)[C@@H](O)[C@H](O)[C@@H]2O)c1] | 29 |
| 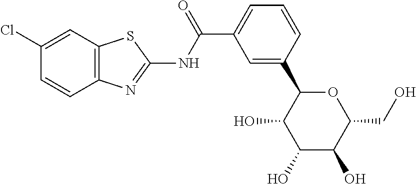 [SMILES: O=C(Nc1nc2ccc(Cl)cc2s1)c1cccc([C@H]2O[C@H](CO)[C@@H](O)[C@H](O)[C@@H]2O)c1] | 30 |
| 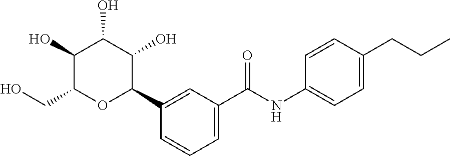 [SMILES: CCCc1ccc(NC(=O)c2cccc([C@H]3O[C@H](CO)[C@@H](O)[C@H](O)[C@@H]3O)c2)cc1] | 31 |
| 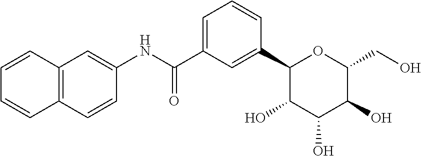 [SMILES: O=C(Nc1ccc2ccccc2c1)c1cccc([C@H]2O[C@H](CO)[C@@H](O)[C@H](O)[C@@H]2O)c1] | 32 |
| 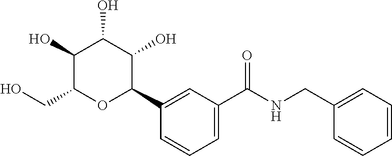 [SMILES: O=C(NCc1ccccc1)c1cccc([C@H]2O[C@H](CO)[C@@H](O)[C@H](O)[C@@H]2O)c1] | 33 |
| 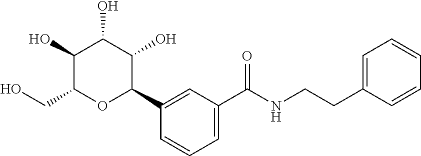 [SMILES: O=C(NCCc1ccccc1)c1cccc([C@H]2O[C@H](CO)[C@@H](O)[C@H](O)[C@@H]2O)c1] | 34 |
| 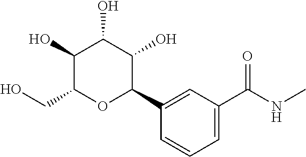 [SMILES: CNC(=O)c1cccc([C@H]2O[C@H](CO)[C@@H](O)[C@H](O)[C@@H]2O)c1] | 35 |
| 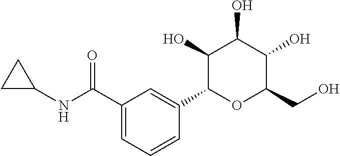 [SMILES: O=C(NC1CC1)c1cccc([C@H]2O[C@H](CO)[C@@H](O)[C@H](O)[C@@H]2O)c1] | 36 |
| 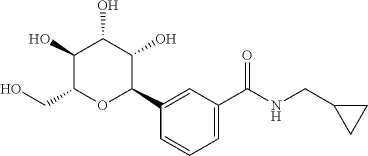 [SMILES: O=C(NCC1CC1)c1cccc([C@H]2O[C@H](CO)[C@@H](O)[C@H](O)[C@@H]2O)c1] | 37 |
| 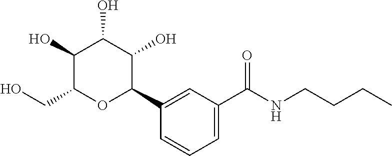 [SMILES: CCCCNC(=O)c1cccc([C@H]2O[C@H](CO)[C@@H](O)[C@H](O)[C@@H]2O)c1] | 38 |
| 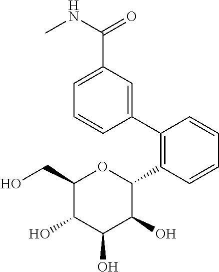 [SMILES: CNC(=O)c1cccc(-c2ccccc2[C@H]2O[C@H](CO)[C@@H](O)[C@H](O)[C@@H]2O)c1] | 39 |
| 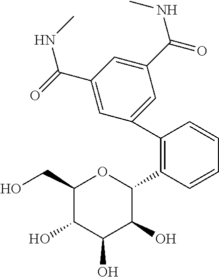 [SMILES: CNC(=O)c1cc(C(=O)NC)cc(-c2ccccc2[C@H]2O[C@H](CO)[C@@H](O)[C@H](O)[C@@H]2O)c1] | 40 |
| 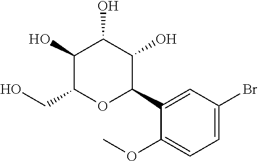 [SMILES: COc1ccc(Br)cc1[C@H]1O[C@H](CO)[C@@H](O)[C@H](O)[C@@H]1O] | 41 |
| 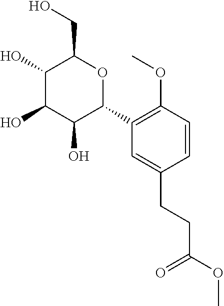 [SMILES: COC(=O)CCc1ccc(OC)c([C@H]2O[C@H](CO)[C@@H](O)[C@H](O)[C@@H]2O)c1] | 42 |
| 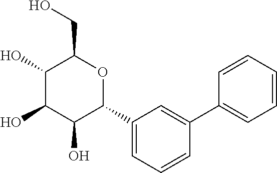 [SMILES: OC[C@H]1O[C@H](c2cccc(-c3ccccc3)c2)[C@@H](O)[C@@H](O)[C@@H]1O] | 43 |
| 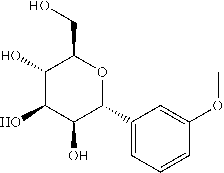 [SMILES: COc1cccc([C@H]2O[C@H](CO)[C@@H](O)[C@H](O)[C@@H]2O)c1] | 44 |
| 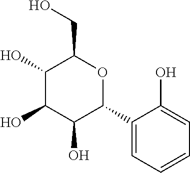 [SMILES: OC[C@H]1O[C@H](c2ccccc2O)[C@@H](O)[C@@H](O)[C@@H]1O] | 45 |
| 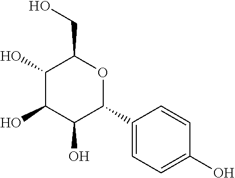 [SMILES: OC[C@H]1O[C@H](c2ccc(O)cc2)[C@@H](O)[C@@H](O)[C@@H]1O] | 46 |
| 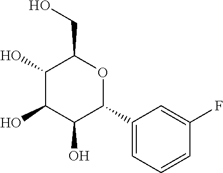 [SMILES: OC[C@H]1O[C@H](c2cccc(F)c2)[C@@H](O)[C@@H](O)[C@@H]1O] | 47 |
| 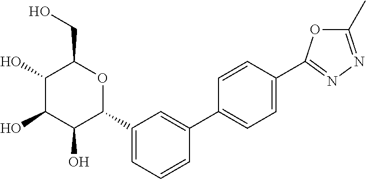 [SMILES: Cc1nnc(-c2ccc(-c3cccc([C@H]4O[C@H](CO)[C@@H](O)[C@H](O)[C@@H]4O)c3)cc2)o1] | 48 |
| 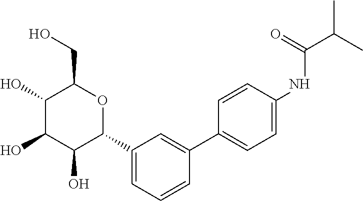 [SMILES: CC(C)C(=O)Nc1ccc(-c2cccc([C@H]3O[C@H](CO)[C@@H](O)[C@H](O)[C@@H]3O)c2)cc1] | 49 |
| 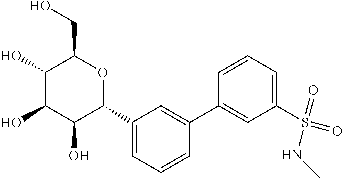 [SMILES: CNS(=O)(=O)c1cccc(-c2cccc([C@H]3O[C@H](CO)[C@@H](O)[C@H](O)[C@@H]3O)c2)c1] | 50 |
| 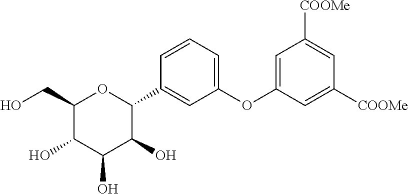 [SMILES: COC(=O)c1cc(Oc2cccc([C@H]3O[C@H](CO)[C@@H](O)[C@H](O)[C@@H]3O)c2)cc(C(=O)OC)c1] | 51 |
| 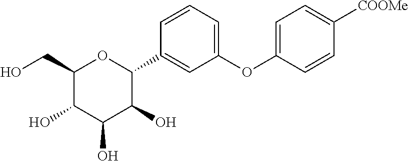 [SMILES: COC(=O)c1ccc(Oc2cccc([C@H]3O[C@H](CO)[C@@H](O)[C@H](O)[C@@H]3O)c2)cc1] | 52 |
| 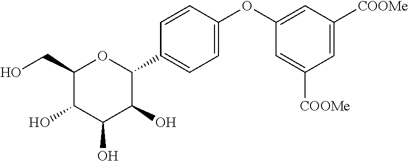 [SMILES: COC(=O)c1cc(Oc2ccc([C@H]3O[C@H](CO)[C@@H](O)[C@H](O)[C@@H]3O)cc2)cc(C(=O)OC)c1] | 53 |
| 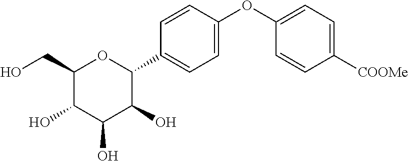 [SMILES: COC(=O)c1ccc(Oc2ccc([C@H]3O[C@H](CO)[C@@H](O)[C@H](O)[C@@H]3O)cc2)cc1] | 54 |
| 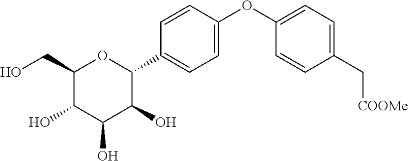 [SMILES: COC(=O)Cc1ccc(Oc2ccc([C@H]3O[C@H](CO)[C@@H](O)[C@H](O)[C@@H]3O)cc2)cc1] | 55 |
| 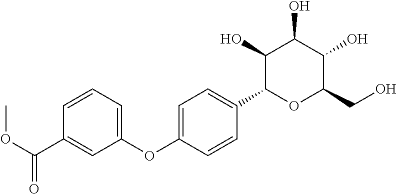 [SMILES: COC(=O)c1cccc(Oc2ccc([C@H]3O[C@H](CO)[C@@H](O)[C@H](O)[C@@H]3O)cc2)c1] | 56 |
| 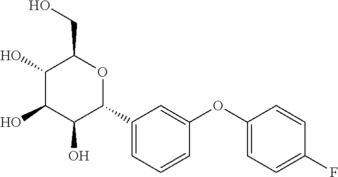 [SMILES: OC[C@H]1O[C@H](c2cccc(Oc3ccc(F)cc3)c2)[C@@H](O)[C@@H](O)[C@@H]1O] | 57 |
| 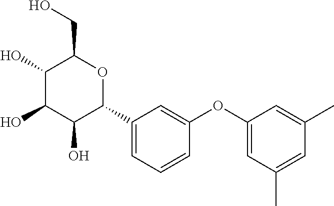 [SMILES: Cc1cc(C)cc(Oc2cccc([C@H]3O[C@H](CO)[C@@H](O)[C@H](O)[C@@H]3O)c2)c1] | 58 |
| 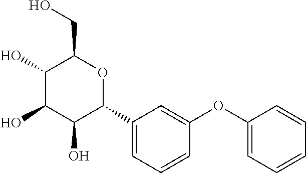 [SMILES: OC[C@H]1O[C@H](c2cccc(Oc3ccccc3)c2)[C@@H](O)[C@@H](O)[C@@H]1O] | 59 |
| 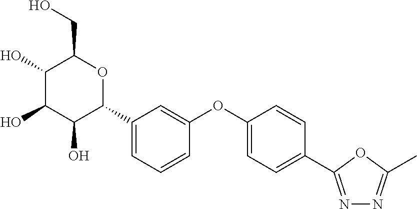 [SMILES: Cc1nnc(-c2ccc(Oc3cccc([C@H]4O[C@H](CO)[C@@H](O)[C@H](O)[C@@H]4O)c3)cc2)o1] | 60 |
| 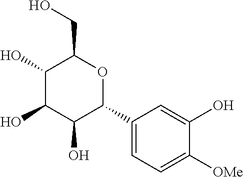 [SMILES: COc1ccc([C@H]2O[C@H](CO)[C@@H](O)[C@H](O)[C@@H]2O)cc1O] | 61 |
| 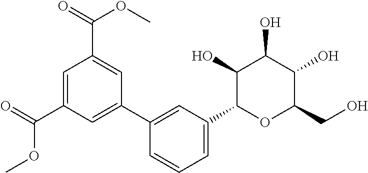 [SMILES: COC(=O)c1cc(C(=O)OC)cc(-c2cccc([C@H]3O[C@H](CO)[C@@H](O)[C@H](O)[C@@H]3O)c2)c1] | 62 |
| 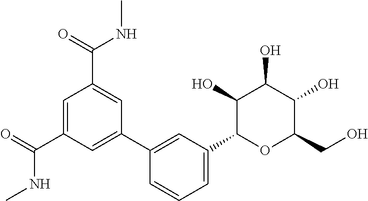 [SMILES: CNC(=O)c1cc(C(=O)NC)cc(-c2cccc([C@H]3O[C@H](CO)[C@@H](O)[C@H](O)[C@@H]3O)c2)c1] | 63 |
| 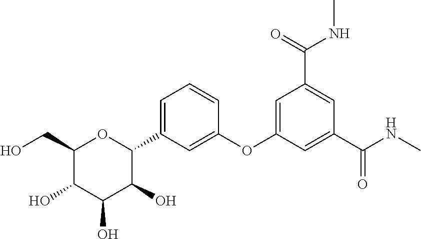 [SMILES: CNC(=O)c1cc(Oc2cccc([C@H]3O[C@H](CO)[C@@H](O)[C@H](O)[C@@H]3O)c2)cc(C(=O)NC)c1] | 64 |
| 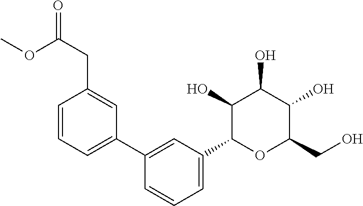 [SMILES: COC(=O)Cc1cccc(-c2cccc([C@H]3O[C@H](CO)[C@@H](O)[C@H](O)[C@@H]3O)c2)c1] | 65 |
| 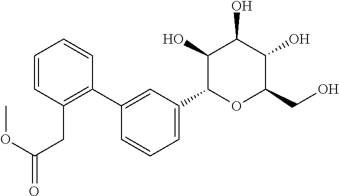 [SMILES: COC(=O)Cc1ccccc1-c1cccc([C@H]2O[C@H](CO)[C@@H](O)[C@H](O)[C@@H]2O)c1] | 66 |
| 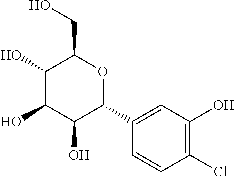 [SMILES: OC[C@H]1O[C@H](c2ccc(Cl)c(O)c2)[C@@H](O)[C@@H](O)[C@@H]1O] | 67 |
| 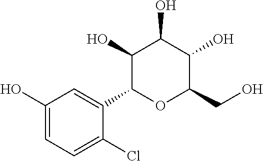 [SMILES: OC[C@H]1O[C@H](c2cc(O)ccc2Cl)[C@@H](O)[C@@H](O)[C@@H]1O] | 68 |
| 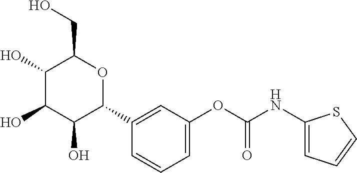 [SMILES: O=C(Nc1cccs1)Oc1cccc([C@H]2O[C@H](CO)[C@@H](O)[C@H](O)[C@@H]2O)c1] | 69 |
| 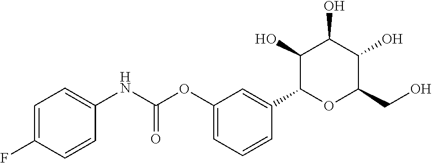 [SMILES: O=C(Nc1ccc(F)cc1)Oc1cccc([C@H]2O[C@H](CO)[C@@H](O)[C@H](O)[C@@H]2O)c1] | 70 |
| 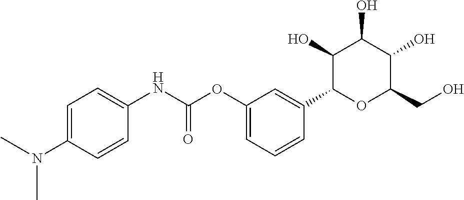 [SMILES: CN(C)c1ccc(NC(=O)Oc2cccc([C@H]3O[C@H](CO)[C@@H](O)[C@H](O)[C@@H]3O)c2)cc1] | 71 |
| 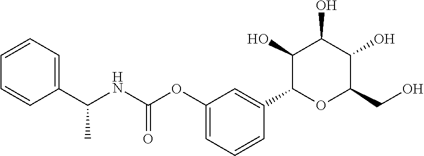 [SMILES: C[C@@H](NC(=O)Oc1cccc([C@H]2O[C@H](CO)[C@@H](O)[C@H](O)[C@@H]2O)c1)c1ccccc1] | 72 |
| 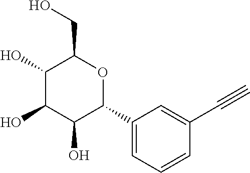 [SMILES: C#Cc1cccc([C@H]2O[C@H](CO)[C@@H](O)[C@H](O)[C@@H]2O)c1] | 73 |
| 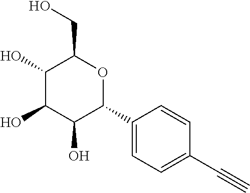 [SMILES: C#Cc1ccc([C@H]2O[C@H](CO)[C@@H](O)[C@H](O)[C@@H]2O)cc1] | 74 |
| 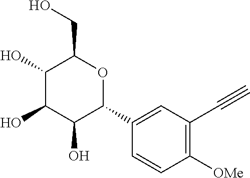 [SMILES: C#Cc1cc([C@H]2O[C@H](CO)[C@@H](O)[C@H](O)[C@@H]2O)ccc1OC] | 75 |
| 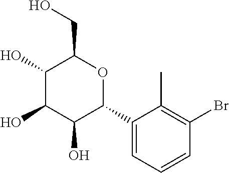 [SMILES: Cc1c(Br)cccc1[C@H]1O[C@H](CO)[C@@H](O)[C@H](O)[C@@H]1O] | 76 |
| 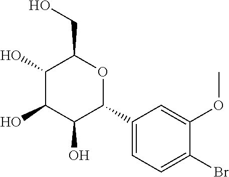 [SMILES: COc1cc([C@H]2O[C@H](CO)[C@@H](O)[C@H](O)[C@@H]2O)ccc1Br] | 77 |
| 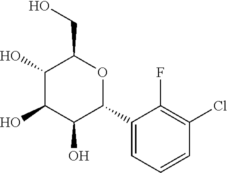 [SMILES: OC[C@H]1O[C@H](c2cccc(Cl)c2F)[C@@H](O)[C@@H](O)[C@@H]1O] | 78 |
| 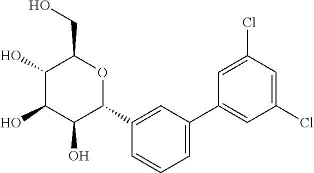 [SMILES: OC[C@H]1O[C@H](c2cccc(-c3cc(Cl)cc(Cl)c3)c2)[C@@H](O)[C@@H](O)[C@@H]1O] | 79 |
| 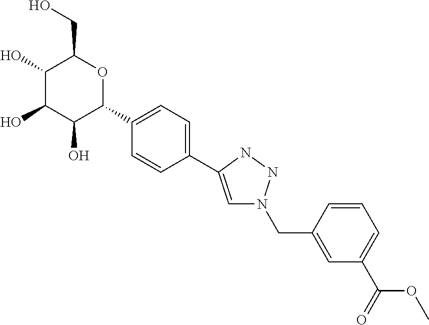 [SMILES: COC(=O)c1cccc(CN2C=C(c3ccc([C@H]4O[C@H](CO)[C@@H](O)[C@H](O)[C@@H]4O)cc3)NN2)c1] | 80 |
| 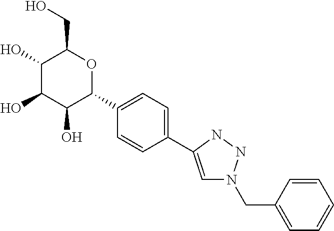 [SMILES: OC[C@H]1O[C@H](c2ccc(C3=CN(Cc4ccccc4)NN3)cc2)[C@@H](O)[C@@H](O)[C@@H]1O] | 81 |
| 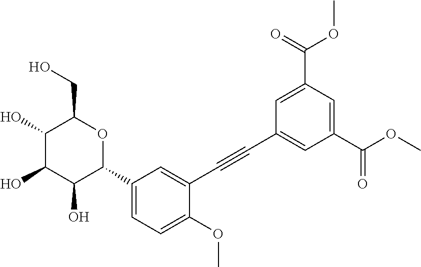 [SMILES: COC(=O)c1cc(C#Cc2cc([C@H]3O[C@H](CO)[C@@H](O)[C@H](O)[C@@H]3O)ccc2OC)cc(C(=O)OC)c1] | 82 |
| 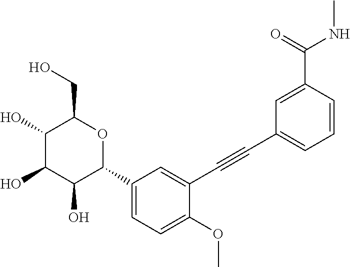 [SMILES: CNC(=O)c1cccc(C#Cc2cc([C@H]3O[C@H](CO)[C@@H](O)[C@H](O)[C@@H]3O)ccc2OC)c1] | 83 |
| 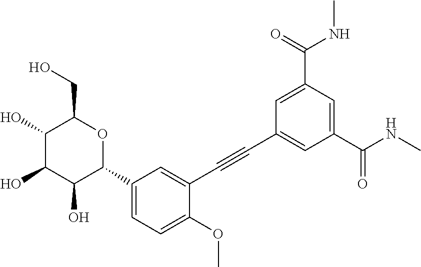 [SMILES: CNC(=O)c1cc(C#Cc2cc([C@H]3O[C@H](CO)[C@@H](O)[C@H](O)[C@@H]3O)ccc2OC)cc(C(=O)NC)c1] | 84 |
| 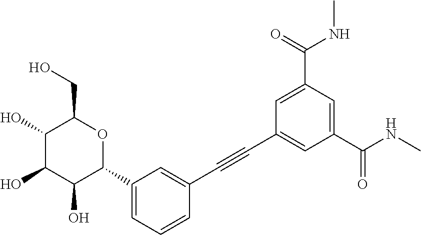 [SMILES: CNC(=O)c1cc(C#Cc2cccc([C@H]3O[C@H](CO)[C@@H](O)[C@H](O)[C@@H]3O)c2)cc(C(=O)NC)c1] | 85 |
| 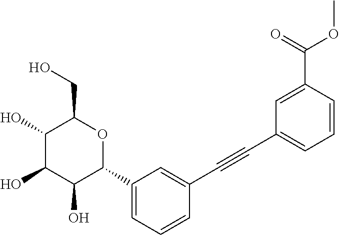 [SMILES: COC(=O)c1cccc(C#Cc2cccc([C@H]3O[C@H](CO)[C@@H](O)[C@H](O)[C@@H]3O)c2)c1] | 86 |
| 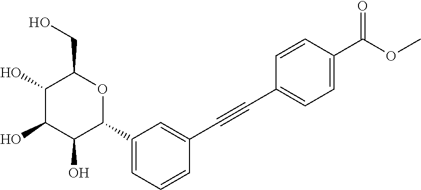 [SMILES: COC(=O)c1ccc(C#Cc2cccc([C@H]3O[C@H](CO)[C@@H](O)[C@H](O)[C@@H]3O)c2)cc1] | 87 |
| 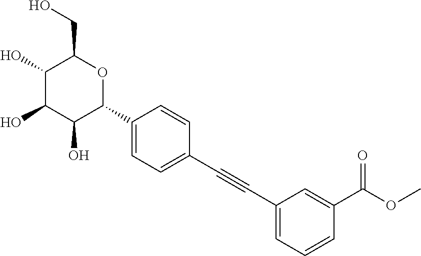 [SMILES: COC(=O)c1cccc(C#Cc2ccc([C@H]3O[C@H](CO)[C@@H](O)[C@H](O)[C@@H]3O)cc2)c1] | 88 |
| 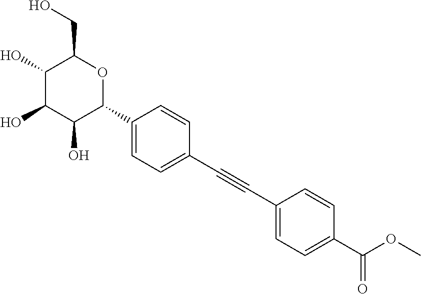 [SMILES: COC(=O)c1ccc(C#Cc2ccc([C@H]3O[C@H](CO)[C@@H](O)[C@H](O)[C@@H]3O)cc2)cc1] | 89 |
| 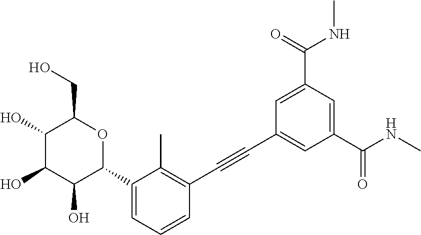 [SMILES: CNC(=O)c1cc(C#Cc2cccc([C@H]3O[C@H](CO)[C@@H](O)[C@H](O)[C@@H]3O)c2C)cc(C(=O)NC)c1] | 90 |
| 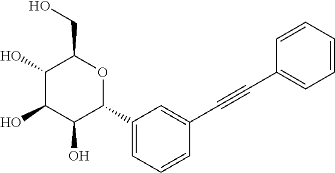 [SMILES: OC[C@H]1O[C@H](c2cccc(C#Cc3ccccc3)c2)[C@@H](O)[C@@H](O)[C@@H]1O] | 91 |
| 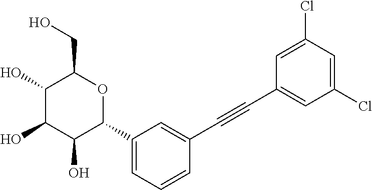 [SMILES: OC[C@H]1O[C@H](c2cccc(C#Cc3cc(Cl)cc(Cl)c3)c2)[C@@H](O)[C@@H](O)[C@@H]1O] | 92 |
| 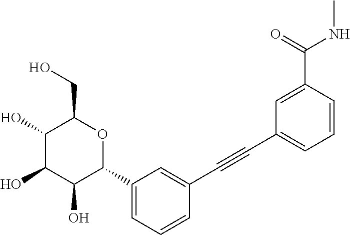 [SMILES: CNC(=O)c1cccc(C#Cc2cccc([C@H]3O[C@H](CO)[C@@H](O)[C@H](O)[C@@H]3O)c2)c1] | 93 |
| 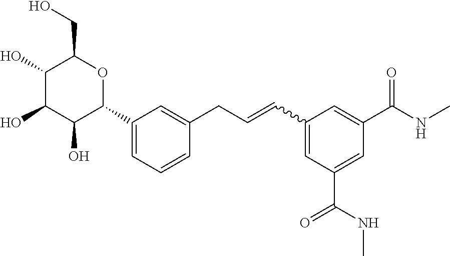 [SMILES: CNC(=O)c1cc(C=CCc2cccc([C@H]3O[C@H](CO)[C@@H](O)[C@H](O)[C@@H]3O)c2)cc(C(=O)NC)c1] | 94 |
| 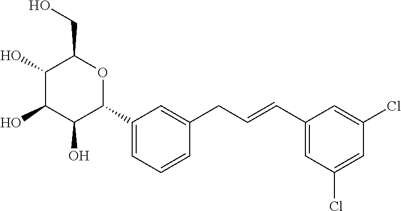 [SMILES: OC[C@H]1O[C@H](c2cccc(C/C=C/c3cc(Cl)cc(Cl)c3)c2)[C@@H](O)[C@@H](O)[C@@H]1O] | 95 |
| 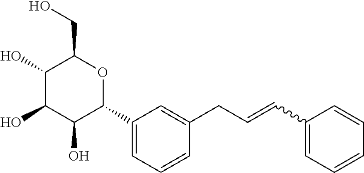 [SMILES: OC[C@H]1O[C@H](c2cccc(CC=Cc3ccccc3)c2)[C@@H](O)[C@@H](O)[C@@H]1O] | 96 |
| 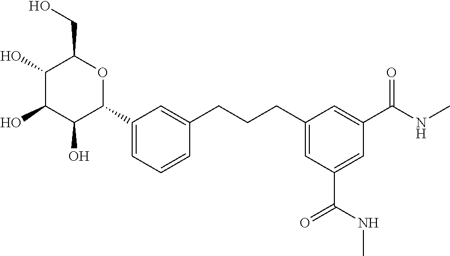 [SMILES: CNC(=O)c1cc(CCCc2cccc([C@H]3O[C@H](CO)[C@@H](O)[C@H](O)[C@@H]3O)c2)cc(C(=O)NC)c1] | 97 |
| 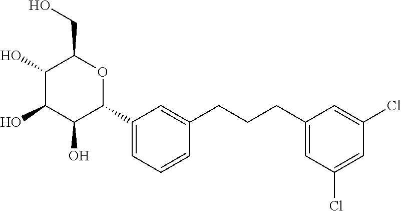 [SMILES: OC[C@H]1O[C@H](c2cccc(CCCc3cc(Cl)cc(Cl)c3)c2)[C@@H](O)[C@@H](O)[C@@H]1O] | 98 |
| 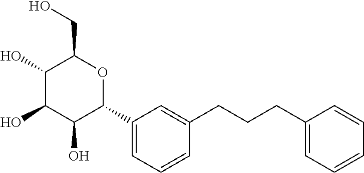 [SMILES: OC[C@H]1O[C@H](c2cccc(CCCc3ccccc3)c2)[C@@H](O)[C@@H](O)[C@@H]1O] | 99 |
| 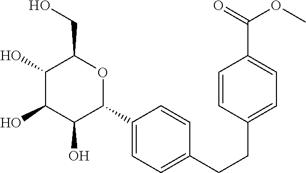 [SMILES: COC(=O)c1ccc(CCc2ccc([C@H]3O[C@H](CO)[C@@H](O)[C@H](O)[C@@H]3O)cc2)cc1] | 100 |
| 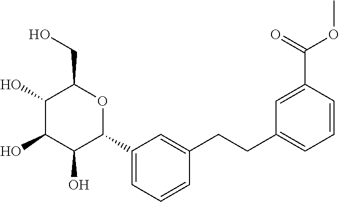 [SMILES: COC(=O)c1cccc(CCc2cccc([C@H]3O[C@H](CO)[C@@H](O)[C@H](O)[C@@H]3O)c2)c1] | 101 |
| 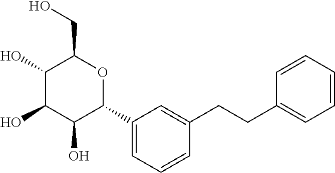 [SMILES: OC[C@H]1O[C@H](c2cccc(CCc3ccccc3)c2)[C@@H](O)[C@@H](O)[C@@H]1O] | 102 |
| 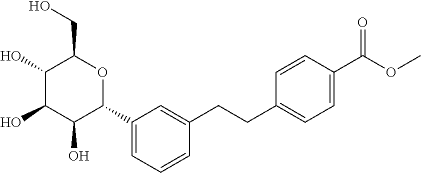 [SMILES: COC(=O)c1ccc(CCc2cccc([C@H]3O[C@H](CO)[C@@H](O)[C@H](O)[C@@H]3O)c2)cc1] | 103 |
| 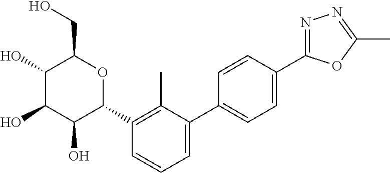 [SMILES: Cc1nnc(-c2ccc(-c3cccc([C@H]4O[C@H](CO)[C@@H](O)[C@H](O)[C@@H]4O)c3C)cc2)o1] | 104 |
| 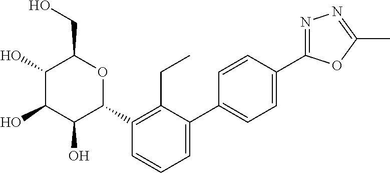 [SMILES: CCc1c(-c2ccc(-c3nnc(C)o3)cc2)cccc1[C@H]1O[C@H](CO)[C@@H](O)[C@H](O)[C@@H]1O] | 105 |
| 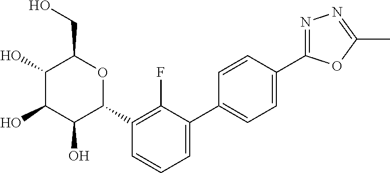 [SMILES: Cc1nnc(-c2ccc(-c3cccc([C@H]4O[C@H](CO)[C@@H](O)[C@H](O)[C@@H]4O)c3F)cc2)o1] | 106 |
| 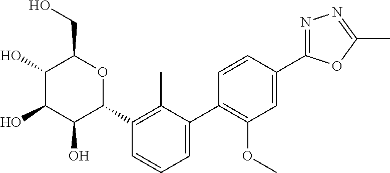 [SMILES: COc1cc(-c2nnc(C)o2)ccc1-c1cccc([C@H]2O[C@H](CO)[C@@H](O)[C@H](O)[C@@H]2O)c1C] | 107 |
| 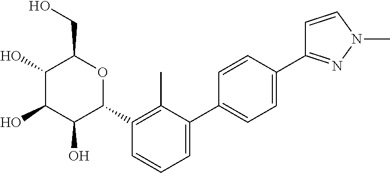 [SMILES: Cc1c(-c2ccc(-c3ccn(C)n3)cc2)cccc1[C@H]1O[C@H](CO)[C@@H](O)[C@H](O)[C@@H]1O] | 108 |
| 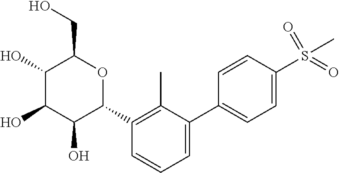 [SMILES: Cc1c(-c2ccc(S(C)(=O)=O)cc2)cccc1[C@H]1O[C@H](CO)[C@@H](O)[C@H](O)[C@@H]1O] | 109 |
| 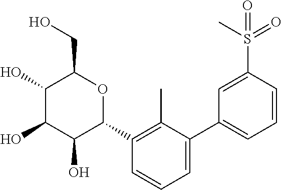 [SMILES: Cc1c(-c2cccc(S(C)(=O)=O)c2)cccc1[C@H]1O[C@H](CO)[C@@H](O)[C@H](O)[C@@H]1O] | 110 |
| 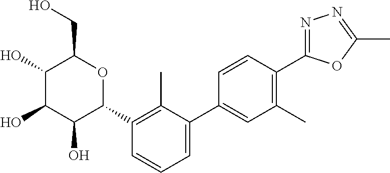 [SMILES: Cc1nnc(-c2ccc(-c3cccc([C@H]4O[C@H](CO)[C@@H](O)[C@H](O)[C@@H]4O)c3C)cc2C)o1] | 111 |
| 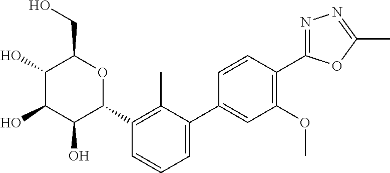 [SMILES: COc1cc(-c2cccc([C@H]3O[C@H](CO)[C@@H](O)[C@H](O)[C@@H]3O)c2C)ccc1-c1nnc(C)o1] | 112 |
| 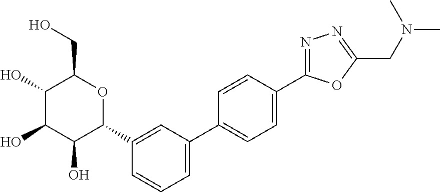 [SMILES: CN(C)Cc1nnc(-c2ccc(-c3cccc([C@H]4O[C@H](CO)[C@@H](O)[C@H](O)[C@@H]4O)c3)cc2)o1] | 113 |
| 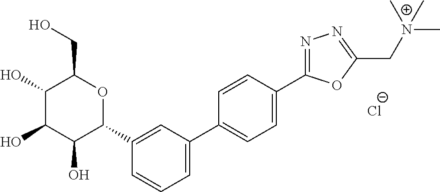 [SMILES: C[N+](C)(C)Cc1nnc(-c2ccc(-c3cccc([C@H]4O[C@H](CO)[C@@H](O)[C@H](O)[C@@H]4O)c3)cc2)o1 ; [Cl-]] | 114 |
| 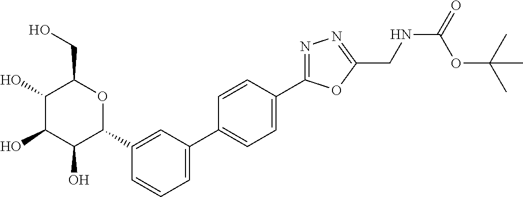 [SMILES: CC(C)(C)OC(=O)NCc1nnc(-c2ccc(-c3cccc([C@H]4O[C@H](CO)[C@@H](O)[C@H](O)[C@@H]4O)c3)cc2)o1] | 115 |
| 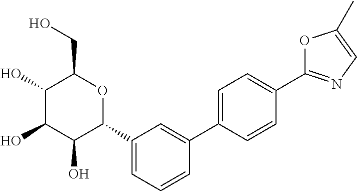 [SMILES: Cc1cnc(-c2ccc(-c3cccc([C@H]4O[C@H](CO)[C@@H](O)[C@H](O)[C@@H]4O)c3)cc2)o1] | 117 |
| 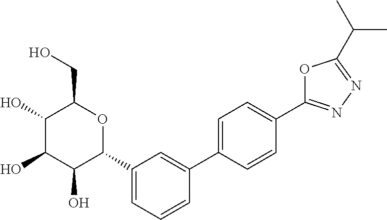 [SMILES: CC(C)c1nnc(-c2ccc(-c3cccc([C@H]4O[C@H](CO)[C@@H](O)[C@H](O)[C@@H]4O)c3)cc2)o1] | 118 |
| 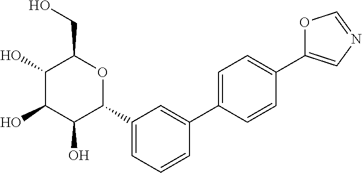 [SMILES: OC[C@H]1O[C@H](c2cccc(-c3ccc(-c4cnco4)cc3)c2)[C@@H](O)[C@@H](O)[C@@H]1O] | 119 |
| 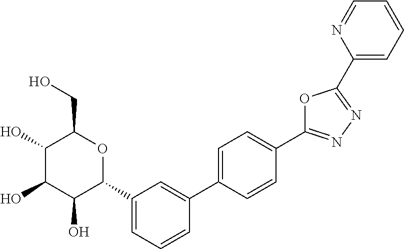 [SMILES: OC[C@H]1O[C@H](c2cccc(-c3ccc(-c4nnc(-c5ccccn5)o4)cc3)c2)[C@@H](O)[C@@H](O)[C@@H]1O] | 120 |
| 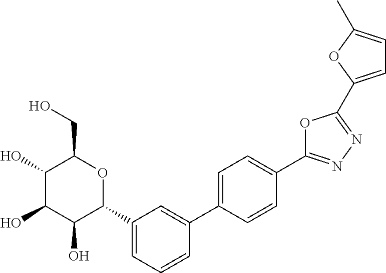 [SMILES: Cc1ccc(-c2nnc(-c3ccc(-c4cccc([C@H]5O[C@H](CO)[C@@H](O)[C@H](O)[C@@H]5O)c4)cc3)o2)o1] | 121 |
| 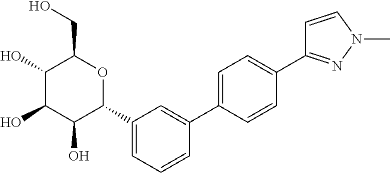 [SMILES: Cn1ccc(-c2ccc(-c3cccc([C@H]4O[C@H](CO)[C@@H](O)[C@H](O)[C@@H]4O)c3)cc2)n1] | 122 |
| 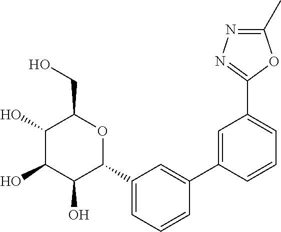 [SMILES: Cc1nnc(-c2cccc(-c3cccc([C@H]4O[C@H](CO)[C@@H](O)[C@H](O)[C@@H]4O)c3)c2)o1] | 123 |
| 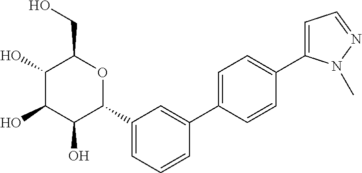 [SMILES: Cn1nccc1-c1ccc(-c2cccc([C@H]3O[C@H](CO)[C@@H](O)[C@H](O)[C@@H]3O)c2)cc1] | 124 |
| 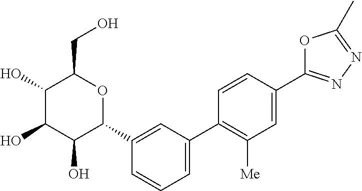 [SMILES: Cc1nnc(-c2ccc(-c3cccc([C@H]4O[C@H](CO)[C@@H](O)[C@H](O)[C@@H]4O)c3)c(C)c2)o1] | 125 |
| 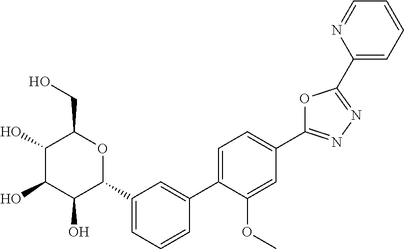 [SMILES: COc1cc(-c2nnc(-c3ccccn3)o2)ccc1-c1cccc([C@H]2O[C@H](CO)[C@@H](O)[C@H](O)[C@@H]2O)c1] | 126 |
| 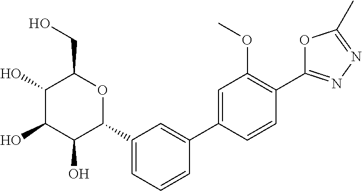 [SMILES: COc1cc(-c2cccc([C@H]3O[C@H](CO)[C@@H](O)[C@H](O)[C@@H]3O)c2)ccc1-c1nnc(C)o1] | 127 |
| 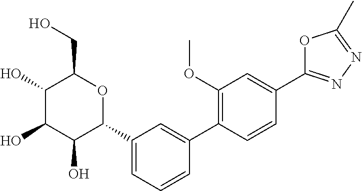 [SMILES: COc1cc(-c2nnc(C)o2)ccc1-c1cccc([C@H]2O[C@H](CO)[C@@H](O)[C@H](O)[C@@H]2O)c1] | 128 |
| 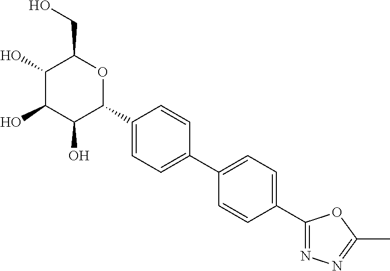 [SMILES: Cc1nnc(-c2ccc(-c3ccc([C@H]4O[C@H](CO)[C@@H](O)[C@H](O)[C@@H]4O)cc3)cc2)o1] | 129 |
| 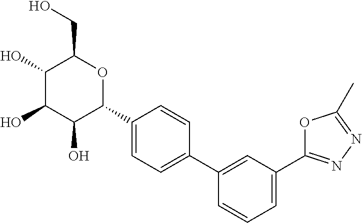 [SMILES: Cc1nnc(-c2cccc(-c3ccc([C@H]4O[C@H](CO)[C@@H](O)[C@H](O)[C@@H]4O)cc3)c2)o1] | 130 |
| 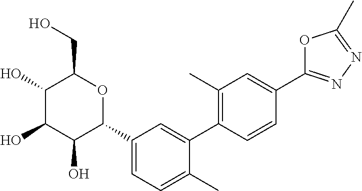 [SMILES: Cc1nnc(-c2ccc(-c3cc([C@H]4O[C@H](CO)[C@@H](O)[C@H](O)[C@@H]4O)ccc3C)c(C)c2)o1] | 131 |
| 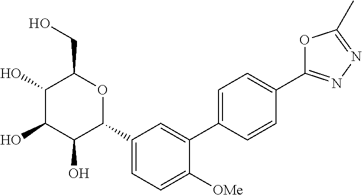 [SMILES: COc1ccc([C@H]2O[C@H](CO)[C@@H](O)[C@H](O)[C@@H]2O)cc1-c1ccc(-c2nnc(C)o2)cc1] | 132 |
| 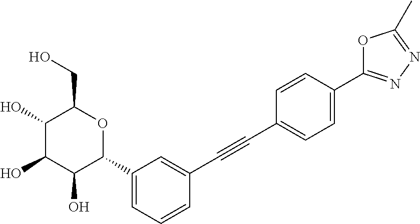 [SMILES: Cc1nnc(-c2ccc(C#Cc3cccc([C@H]4O[C@H](CO)[C@@H](O)[C@H](O)[C@@H]4O)c3)cc2)o1] | 133 |
| 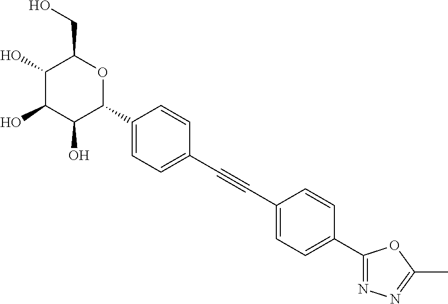 [SMILES: Cc1nnc(-c2ccc(C#Cc3ccc([C@H]4O[C@H](CO)[C@@H](O)[C@H](O)[C@@H]4O)cc3)cc2)o1] | 134 |
| 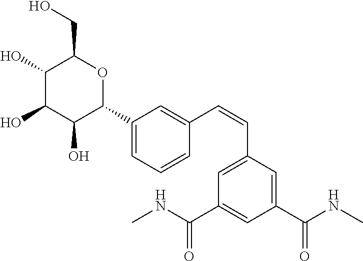 [SMILES: CNC(=O)c1cc(/C=C\c2cccc([C@H]3O[C@H](CO)[C@@H](O)[C@H](O)[C@@H]3O)c2)cc(C(=O)NC)c1] | 135 |
| 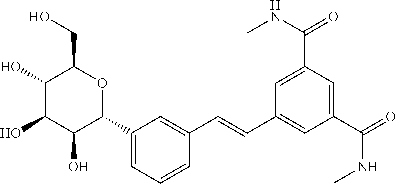 [SMILES: CNC(=O)c1cc(/C=C/c2cccc([C@H]3O[C@H](CO)[C@@H](O)[C@H](O)[C@@H]3O)c2)cc(C(=O)NC)c1] | 136 |
| 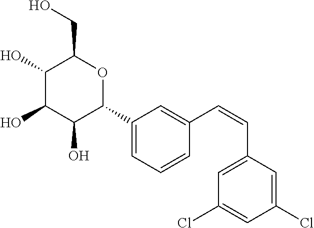 [SMILES: OC[C@H]1O[C@H](c2cccc(/C=C\c3cc(Cl)cc(Cl)c3)c2)[C@@H](O)[C@@H](O)[C@@H]1O] | 137 |
| 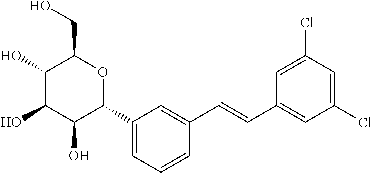 [SMILES: OC[C@H]1O[C@H](c2cccc(/C=C/c3cc(Cl)cc(Cl)c3)c2)[C@@H](O)[C@@H](O)[C@@H]1O] | 138 |
| 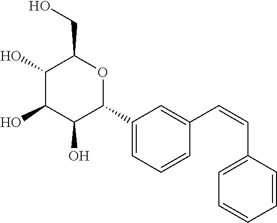 [SMILES: OC[C@H]1O[C@H](c2cccc(/C=C\c3ccccc3)c2)[C@@H](O)[C@@H](O)[C@@H]1O] | 139 |
| 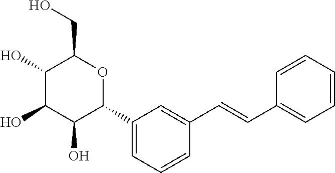 [SMILES: OC[C@H]1O[C@H](c2cccc(/C=C/c3ccccc3)c2)[C@@H](O)[C@@H](O)[C@@H]1O] | 140 |
| 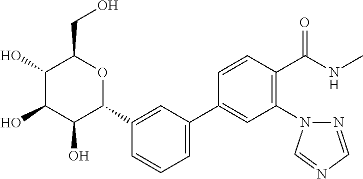 [SMILES: CNC(=O)c1ccc(-c2cccc([C@H]3O[C@H](CO)[C@@H](O)[C@H](O)[C@@H]3O)c2)cc1-n1cncn1] | 141 |
| 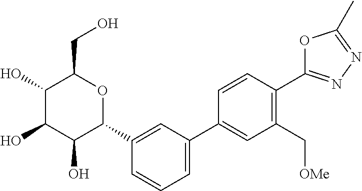 [SMILES: COCc1cc(-c2cccc([C@H]3O[C@H](CO)[C@@H](O)[C@H](O)[C@@H]3O)c2)ccc1-c1nnc(C)o1] | 142 |
| 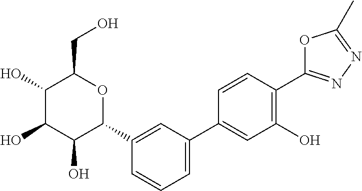 [SMILES: Cc1nnc(-c2ccc(-c3cccc([C@H]4O[C@H](CO)[C@@H](O)[C@H](O)[C@@H]4O)c3)cc2O)o1] | 143 |
| 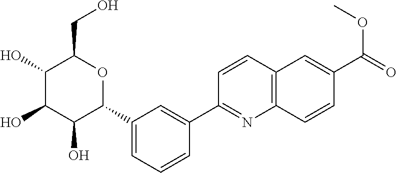 [SMILES: COC(=O)c1ccc2nc(-c3cccc([C@H]4O[C@H](CO)[C@@H](O)[C@H](O)[C@@H]4O)c3)ccc2c1] | 144 |
| 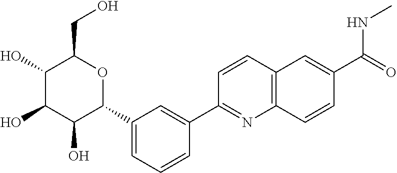 [SMILES: CNC(=O)c1ccc2nc(-c3cccc([C@H]4O[C@H](CO)[C@@H](O)[C@H](O)[C@@H]4O)c3)ccc2c1] | 145 |
| 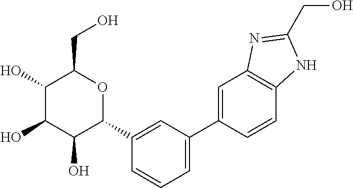 [SMILES: OCc1nc2cc(-c3cccc([C@H]4O[C@H](CO)[C@@H](O)[C@H](O)[C@@H]4O)c3)ccc2[nH]1] | 146 |
| 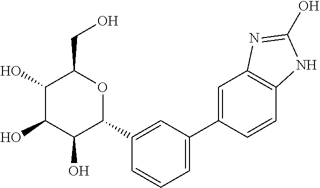 [SMILES: OC[C@H]1O[C@H](c2cccc(-c3ccc4[nH]c(O)nc4c3)c2)[C@@H](O)[C@@H](O)[C@@H]1O] | 147 |
| 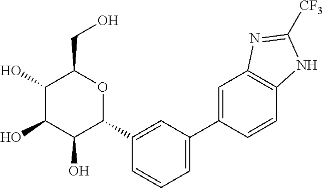 [SMILES: OC[C@H]1O[C@H](c2cccc(-c3ccc4[nH]c(C(F)(F)F)nc4c3)c2)[C@@H](O)[C@@H](O)[C@@H]1O] | 148 |
| 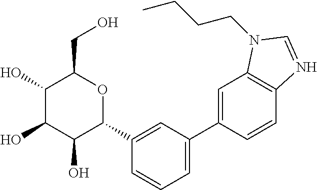 [SMILES: CCCCn1cnc2ccc(-c3cccc([C@H]4O[C@H](CO)[C@@H](O)[C@H](O)[C@@H]4O)c3)cc21] | 149 |
| 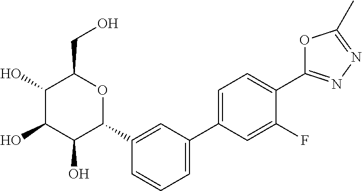 [SMILES: Cc1nnc(-c2ccc(-c3cccc([C@H]4O[C@H](CO)[C@@H](O)[C@H](O)[C@@H]4O)c3)cc2F)o1] | 150 |
| 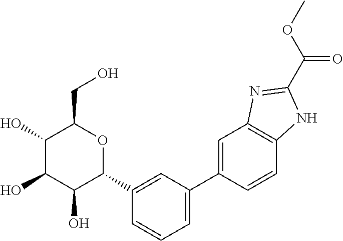 [SMILES: COC(=O)c1nc2cc(-c3cccc([C@H]4O[C@H](CO)[C@@H](O)[C@H](O)[C@@H]4O)c3)ccc2[nH]1] | 151 |
| 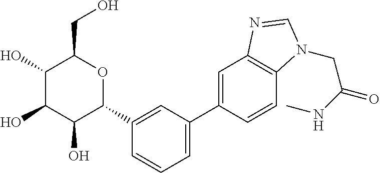 [SMILES: CNC(=O)Cn1cnc2cc(-c3cccc([C@H]4O[C@H](CO)[C@@H](O)[C@H](O)[C@@H]4O)c3)ccc21] | 152 |
| 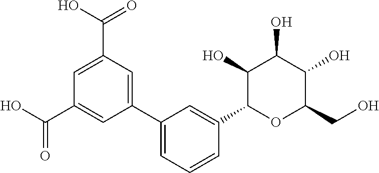 [SMILES: O=C(O)c1cc(C(=O)O)cc(-c2cccc([C@H]3O[C@H](CO)[C@@H](O)[C@H](O)[C@@H]3O)c2)c1] | 153 |
| 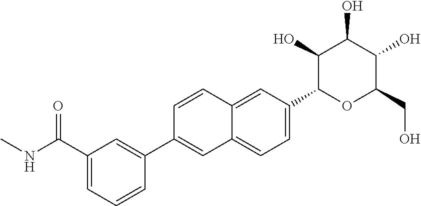 [SMILES: CNC(=O)c1cccc(-c2ccc3cc([C@H]4O[C@H](CO)[C@@H](O)[C@H](O)[C@@H]4O)ccc3c2)c1] | 154 |
| 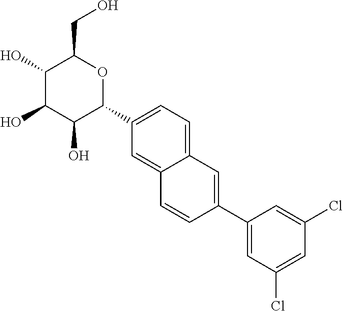 [SMILES: OC[C@H]1O[C@H](c2ccc3cc(-c4cc(Cl)cc(Cl)c4)ccc3c2)[C@@H](O)[C@@H](O)[C@@H]1O] | 155 |
| 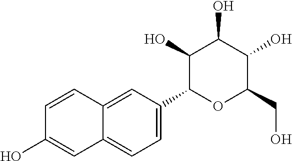 [SMILES: OC[C@H]1O[C@H](c2ccc3cc(O)ccc3c2)[C@@H](O)[C@@H](O)[C@@H]1O] | 156 |
| 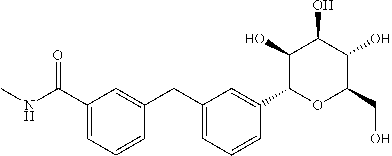 [SMILES: CNC(=O)c1cccc(Cc2cccc([C@H]3O[C@H](CO)[C@@H](O)[C@H](O)[C@@H]3O)c2)c1] | 157 |
| 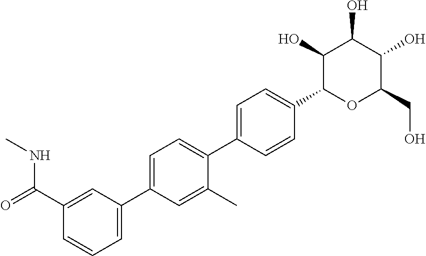 [SMILES: CNC(=O)c1cccc(-c2ccc(-c3ccc([C@H]4O[C@H](CO)[C@@H](O)[C@H](O)[C@@H]4O)cc3)c(C)c2)c1] | 158 |
| 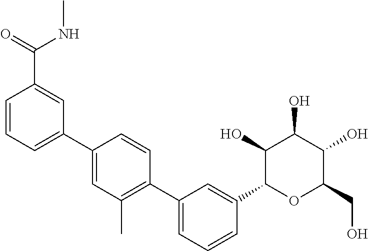 [SMILES: CNC(=O)c1cccc(-c2ccc(-c3cccc([C@H]4O[C@H](CO)[C@@H](O)[C@H](O)[C@@H]4O)c3)c(C)c2)c1] | 159 |
| 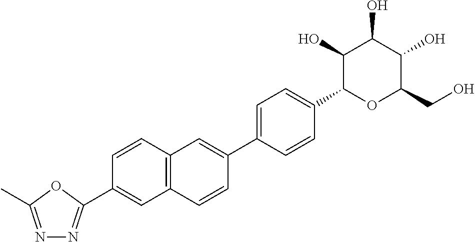 [SMILES: Cc1nnc(-c2ccc3cc(-c4ccc([C@H]5O[C@H](CO)[C@@H](O)[C@H](O)[C@@H]5O)cc4)ccc3c2)o1] | 160 |
| 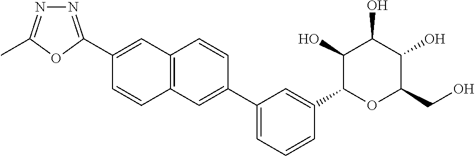 [SMILES: Cc1nnc(-c2ccc3cc(-c4cccc([C@H]5O[C@H](CO)[C@@H](O)[C@H](O)[C@@H]5O)c4)ccc3c2)o1] | 161 |
| 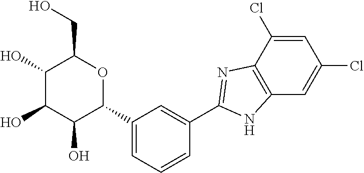 [SMILES: OC[C@H]1O[C@H](c2cccc(-c3nc4c(Cl)cc(Cl)cc4[nH]3)c2)[C@@H](O)[C@@H](O)[C@@H]1O] | 162 |
| 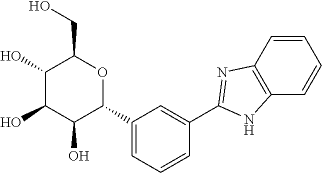 [SMILES: OC[C@H]1O[C@H](c2cccc(-c3nc4ccccc4[nH]3)c2)[C@@H](O)[C@@H](O)[C@@H]1O] | 163 |
| 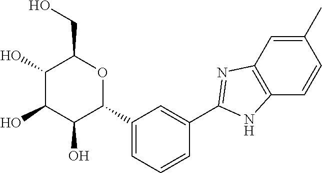 [SMILES: Cc1ccc2[nH]c(-c3cccc([C@H]4O[C@H](CO)[C@@H](O)[C@H](O)[C@@H]4O)c3)nc2c1] | 164 |
| 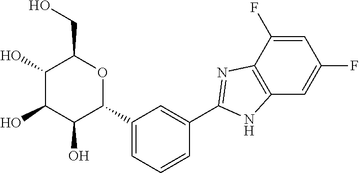 [SMILES: OC[C@H]1O[C@H](c2cccc(-c3nc4c(F)cc(F)cc4[nH]3)c2)[C@@H](O)[C@@H](O)[C@@H]1O] | 165 |
| 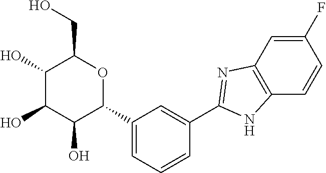 [SMILES: OC[C@H]1O[C@H](c2cccc(-c3nc4cc(F)ccc4[nH]3)c2)[C@@H](O)[C@@H](O)[C@@H]1O] | 166 |
| 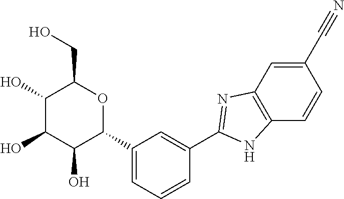 [SMILES: N#Cc1ccc2[nH]c(-c3cccc([C@H]4O[C@H](CO)[C@@H](O)[C@H](O)[C@@H]4O)c3)nc2c1] | 167 |
| 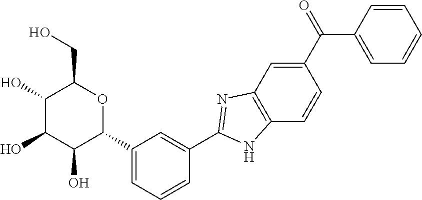 [SMILES: O=C(c1ccccc1)c1ccc2[nH]c(-c3cccc([C@H]4O[C@H](CO)[C@@H](O)[C@H](O)[C@@H]4O)c3)nc2c1] | 168 |
| 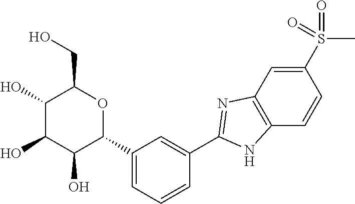 [SMILES: CS(=O)(=O)c1ccc2[nH]c(-c3cccc([C@H]4O[C@H](CO)[C@@H](O)[C@H](O)[C@@H]4O)c3)nc2c1] | 169 |
| 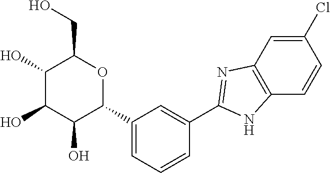 [SMILES: OC[C@H]1O[C@H](c2cccc(-c3nc4cc(Cl)ccc4[nH]3)c2)[C@@H](O)[C@@H](O)[C@@H]1O] | 170 |
| 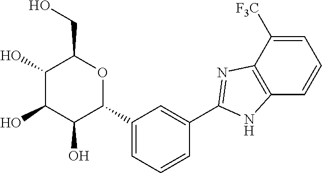 [SMILES: OC[C@H]1O[C@H](c2cccc(-c3nc4c(C(F)(F)F)cccc4[nH]3)c2)[C@@H](O)[C@@H](O)[C@@H]1O] | 171 |
| 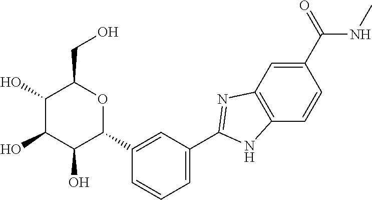 [SMILES: CNC(=O)c1ccc2[nH]c(-c3cccc([C@H]4O[C@H](CO)[C@@H](O)[C@H](O)[C@@H]4O)c3)nc2c1] | 172 |
| 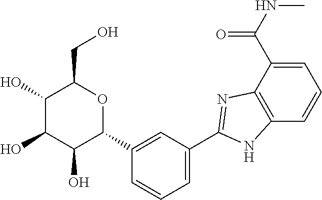 [SMILES: CNC(=O)c1cccc2[nH]c(-c3cccc([C@H]4O[C@H](CO)[C@@H](O)[C@H](O)[C@@H]4O)c3)nc12] | 173 |
| 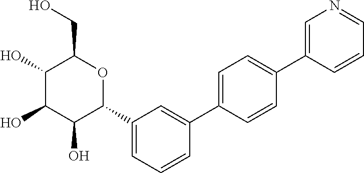 [SMILES: OC[C@H]1O[C@H](c2cccc(-c3ccc(-c4cccnc4)cc3)c2)[C@@H](O)[C@@H](O)[C@@H]1O] | 174 |
| 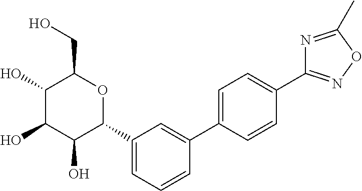 [SMILES: Cc1nc(-c2ccc(-c3cccc([C@H]4O[C@H](CO)[C@@H](O)[C@H](O)[C@@H]4O)c3)cc2)no1] | 175 |
| 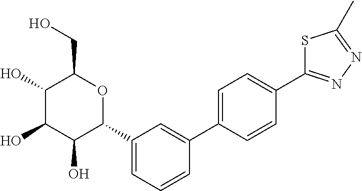 [SMILES: Cc1nnc(-c2ccc(-c3cccc([C@H]4O[C@H](CO)[C@@H](O)[C@H](O)[C@@H]4O)c3)cc2)s1] | 176 |
| 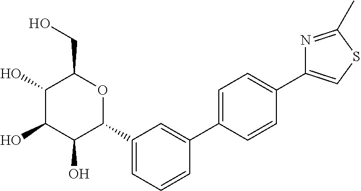 [SMILES: Cc1nc(-c2ccc(-c3cccc([C@H]4O[C@H](CO)[C@@H](O)[C@H](O)[C@@H]4O)c3)cc2)cs1] | 177 |
| 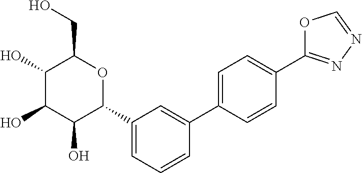 [SMILES: OC[C@H]1O[C@H](c2cccc(-c3ccc(-c4nnco4)cc3)c2)[C@@H](O)[C@@H](O)[C@@H]1O] | 178 |
| 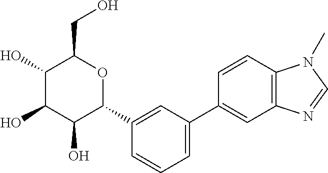 [SMILES: Cn1cnc2cc(-c3cccc([C@H]4O[C@H](CO)[C@@H](O)[C@H](O)[C@@H]4O)c3)ccc21] | 179 |
| 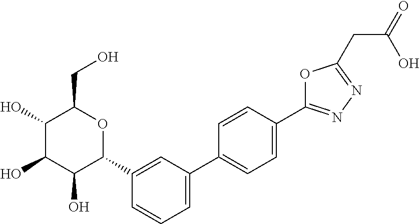 [SMILES: O=C(O)Cc1nnc(-c2ccc(-c3cccc([C@H]4O[C@H](CO)[C@@H](O)[C@H](O)[C@@H]4O)c3)cc2)o1] | 180 |
| 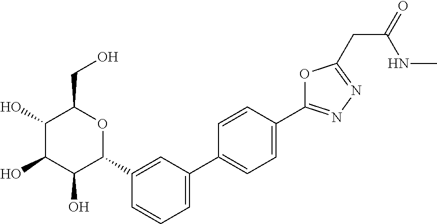 [SMILES: CNC(=O)Cc1nnc(-c2ccc(-c3cccc([C@H]4O[C@H](CO)[C@@H](O)[C@H](O)[C@@H]4O)c3)cc2)o1] | 181 |
| 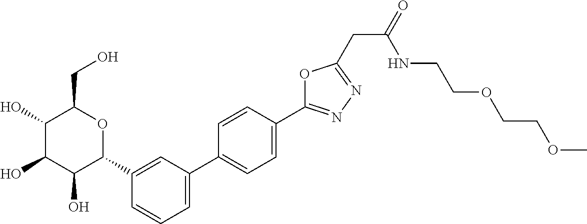 [SMILES: COCCOCCNC(=O)Cc1nnc(-c2ccc(-c3cccc([C@H]4O[C@H](CO)[C@@H](O)[C@H](O)[C@@H]4O)c3)cc2)o1] | 183 |
| 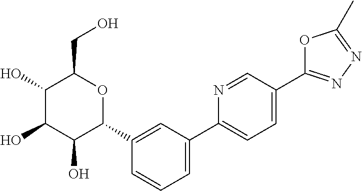 [SMILES: Cc1nnc(-c2ccc(-c3cccc([C@H]4O[C@H](CO)[C@@H](O)[C@H](O)[C@@H]4O)c3)nc2)o1] | 183 |
| 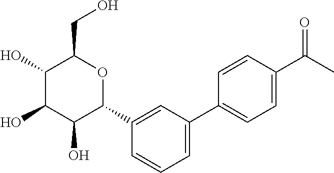 [SMILES: CC(=O)c1ccc(-c2cccc([C@H]3O[C@H](CO)[C@@H](O)[C@H](O)[C@@H]3O)c2)cc1] | 184 |
| 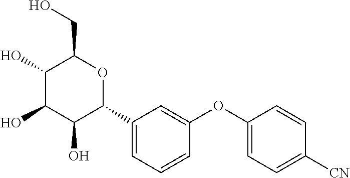 [SMILES: N#Cc1ccc(Oc2cccc([C@H]3O[C@H](CO)[C@@H](O)[C@H](O)[C@@H]3O)c2)cc1] | 185 |
| 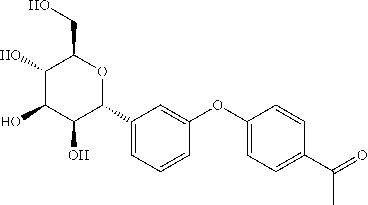 [SMILES: CC(=O)c1ccc(Oc2cccc([C@H]3O[C@H](CO)[C@@H](O)[C@H](O)[C@@H]3O)c2)cc1] | 186 |
| 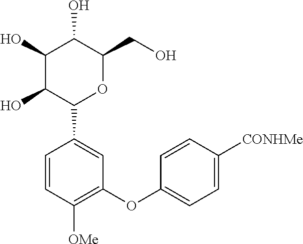 | 187 |
| 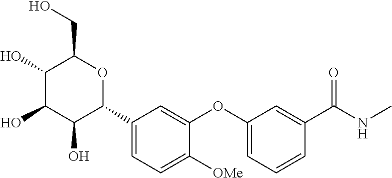 [SMILES: CNC(=O)c1cccc(Oc2cc([C@H]3O[C@H](CO)[C@@H](O)[C@H](O)[C@@H]3O)ccc2OC)c1] | 188 |
| 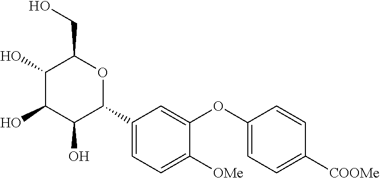 [SMILES: COC(=O)c1ccc(Oc2cc([C@H]3O[C@H](CO)[C@@H](O)[C@H](O)[C@@H]3O)ccc2OC)cc1] | 189 |
| 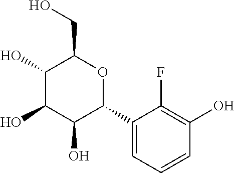 [SMILES: OC[C@H]1O[C@H](c2cccc(O)c2F)[C@@H](O)[C@@H](O)[C@@H]1O] | 190 |
| 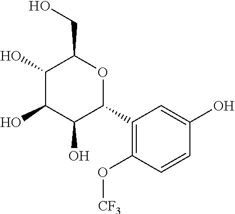 [SMILES: OC[C@H]1O[C@H](c2cc(O)ccc2OC(F)(F)F)[C@@H](O)[C@@H](O)[C@@H]1O] | 191 |
| 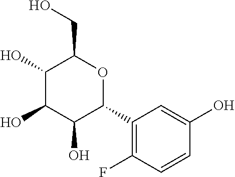 [SMILES: OC[C@H]1O[C@H](c2cc(O)ccc2F)[C@@H](O)[C@@H](O)[C@@H]1O] | 192 |
| 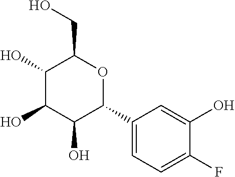 [SMILES: OC[C@H]1O[C@H](c2ccc(F)c(O)c2)[C@@H](O)[C@@H](O)[C@@H]1O] | 193 |
| 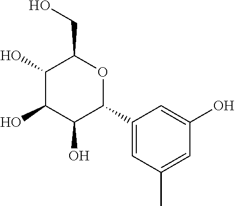 [SMILES: Cc1cc(O)cc([C@H]2O[C@H](CO)[C@@H](O)[C@H](O)[C@@H]2O)c1] | 194 |
| 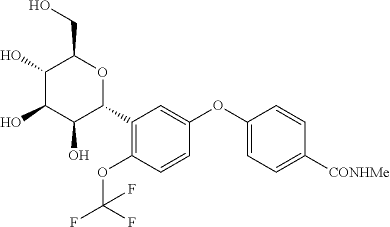 | 195 |
| 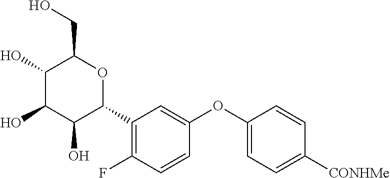 | 196 |
| 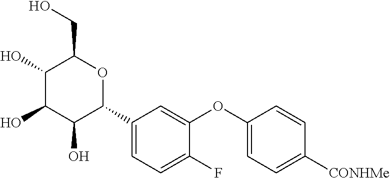 | 197 |
| 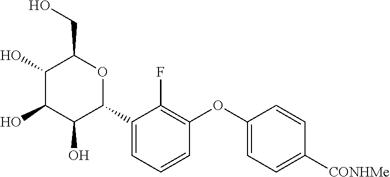 | 198 |
| 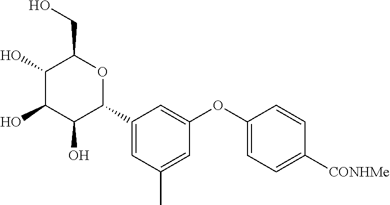 | 199 |
| 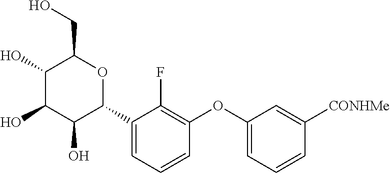 | 200 |
| 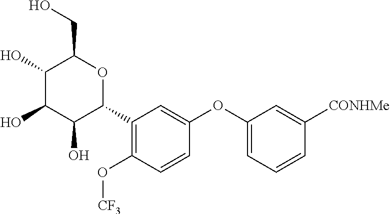 | 201 |
| 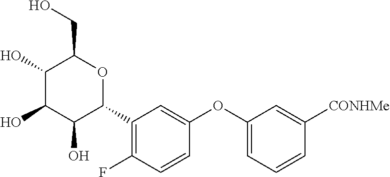 | 202 |
| 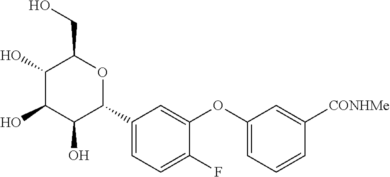 | 203 |
| 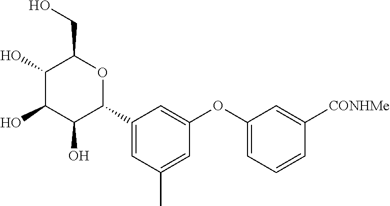 | 204 |
| 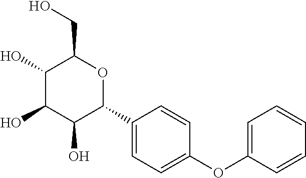 [SMILES: OC[C@H]1O[C@H](c2ccc(Oc3ccccc3)cc2)[C@@H](O)[C@@H](O)[C@@H]1O] | 205 |
| 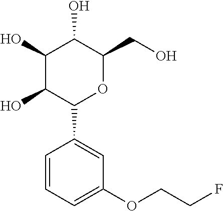 | 206 |
| 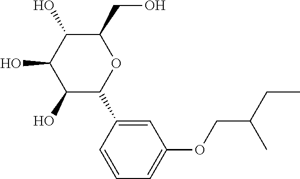 | 207 |
| 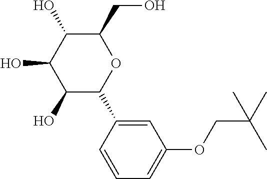 | 208 |
| 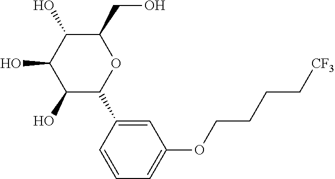 | 209 |
| 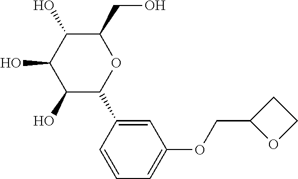 | 210 |
| 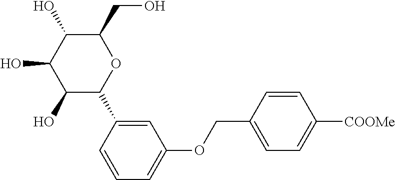 | 211 |
| 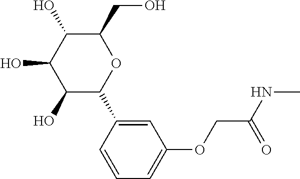 | 212 |
| 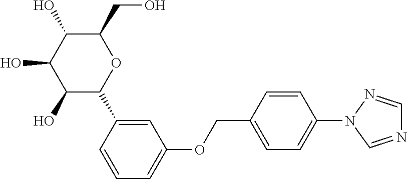 | 213 |
| 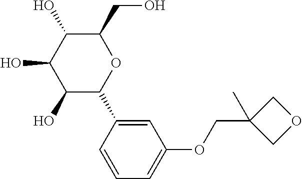 | 214 |
| 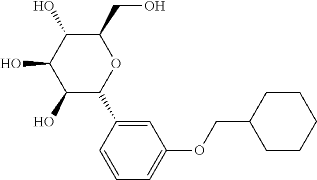 | 215 |
| 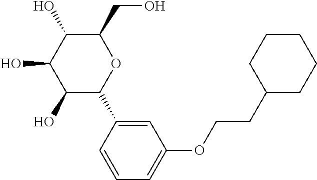 | 216 |
| 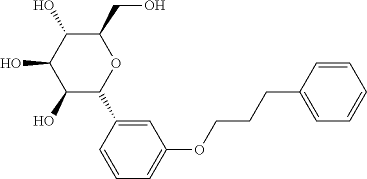 | 217 |
| 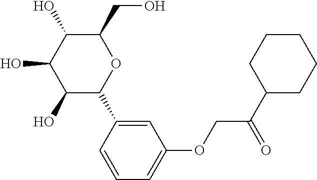 | 218 |
| 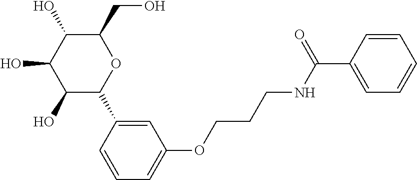 | 219 |
| 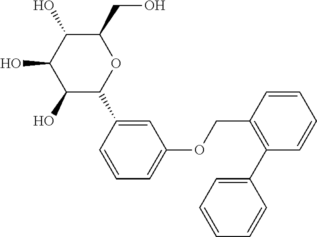 | 220 |
| 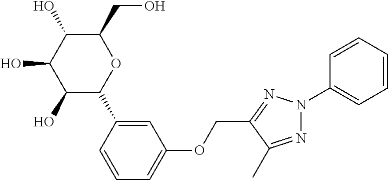 | 221 |
| 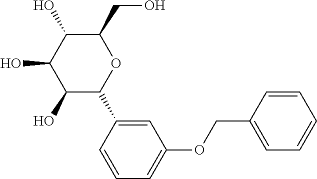 | 222 |
| 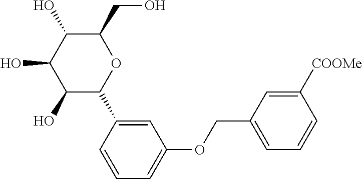 | 223 |
| 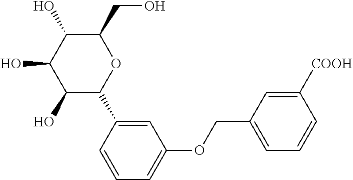 | 224 |
| 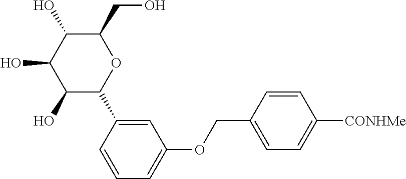 | 225 |
| 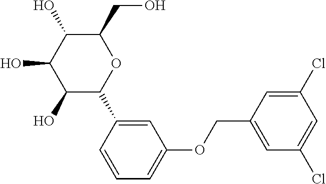 | 226 |
| 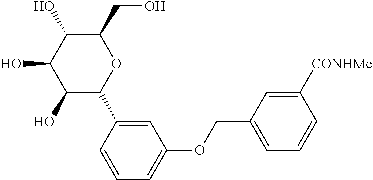 | 227 |
| 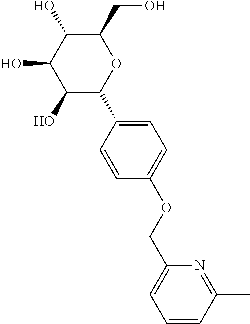 | 228 |
| 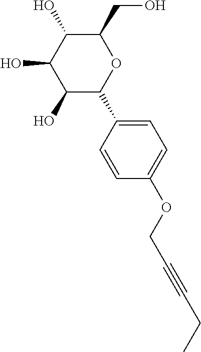 | 229 |
| 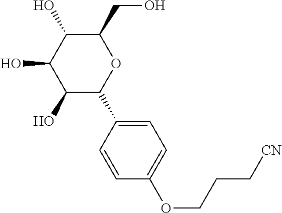 | 230 |
| 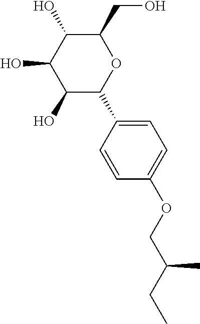 | 231 |
| 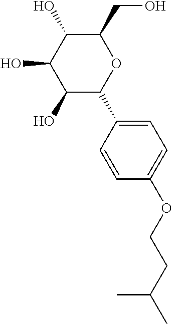 | 232 |
| 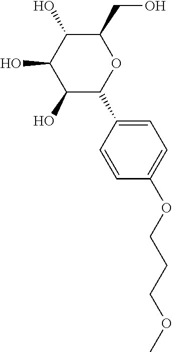 | 233 |
| 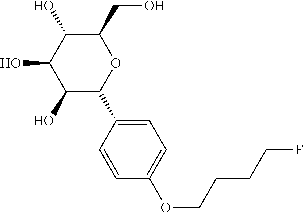 | 234 |
| 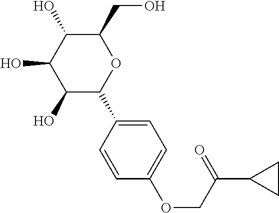 | 235 |
| 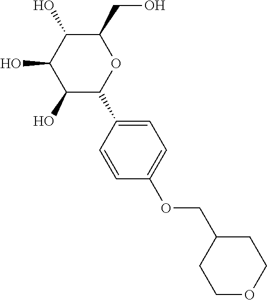 | 236 |
| 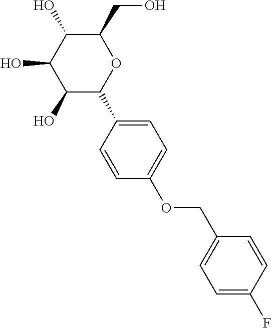 | 237 |
| 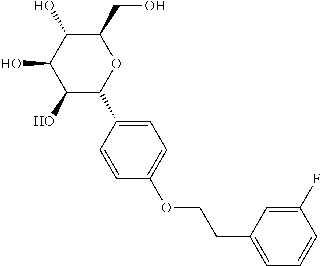 | 238 |
| 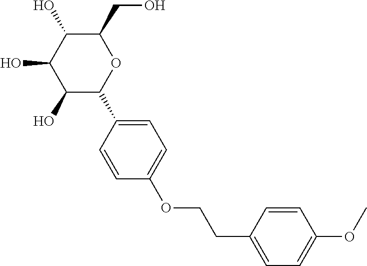 | 239 |
| 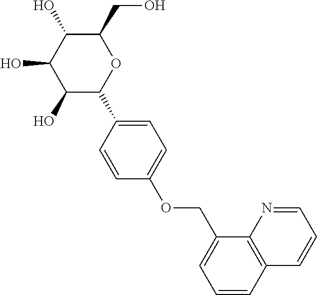 | 240 |
| 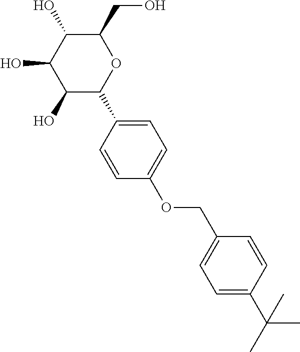 | 241 |
| 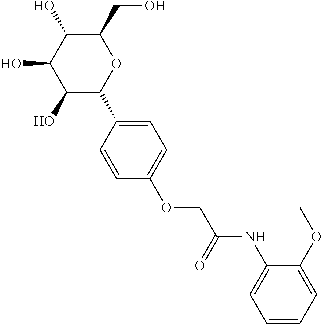 | 242 |
| 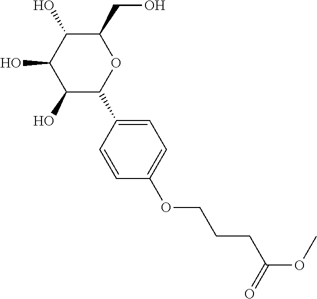 | 243 |
| 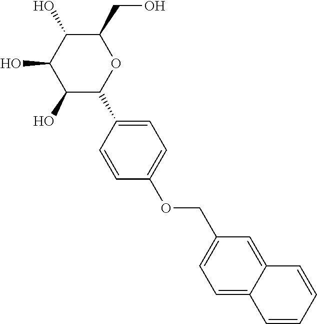 | 244 |
| 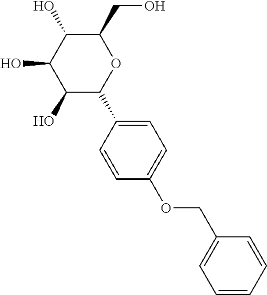 | 245 |
| 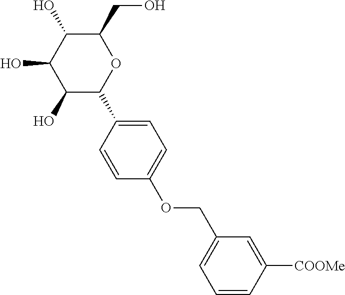 | 246 |
| 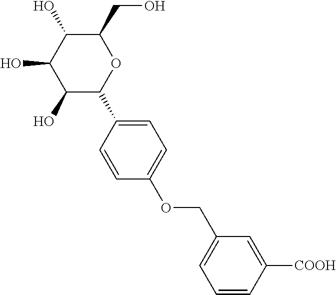 | 247 |
| 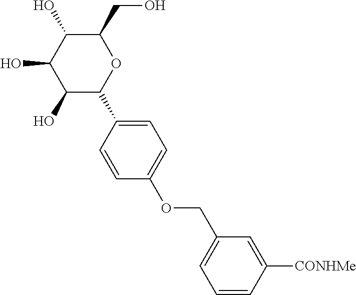 | 248 |
| 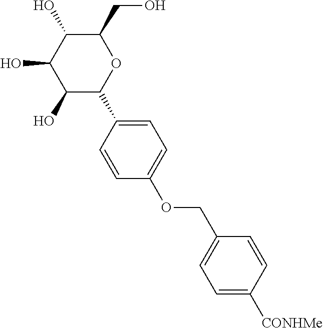 | 249 |
| 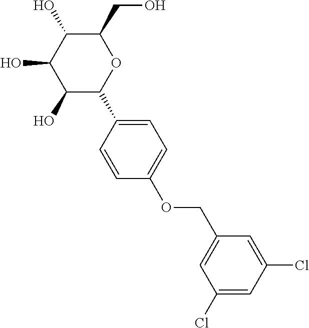 | 250 |
| 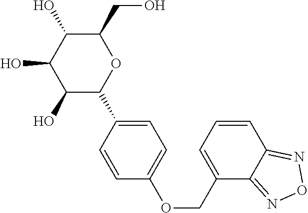 | 251 |
| 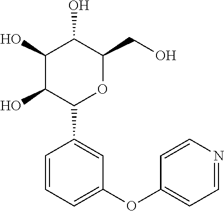 | 252 |
| 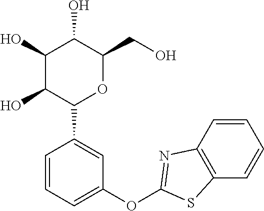 | 253 |
| 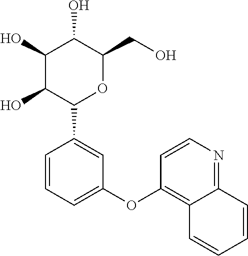 | 254 |
| 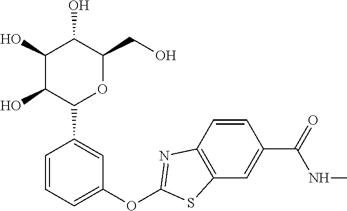 | 255 |
| 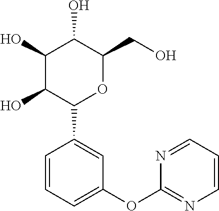 | 256 |
| 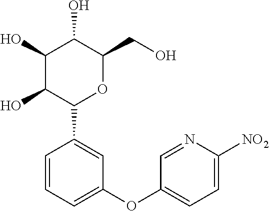 | 257 |
| 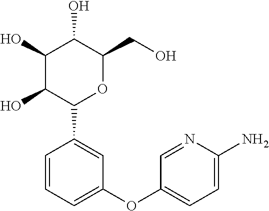 | 258 |
| 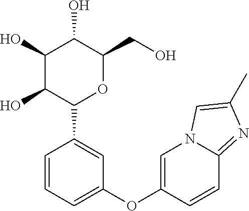 | 259 |
| 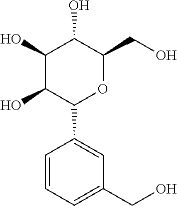 | 260 |
| 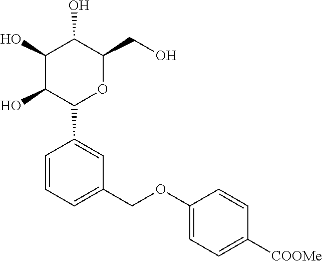 | 261 |
| 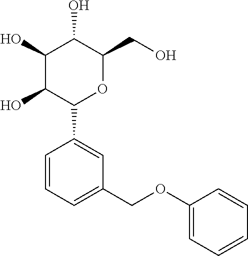 | 262 |
| 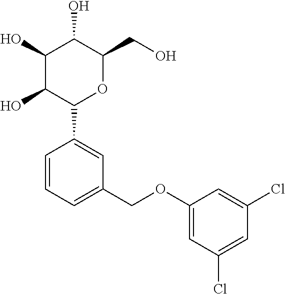 | 263 |
| 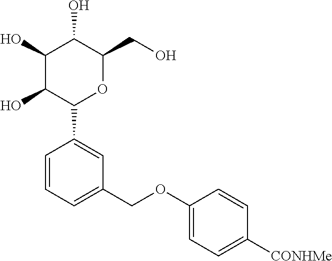 | 264 |
| 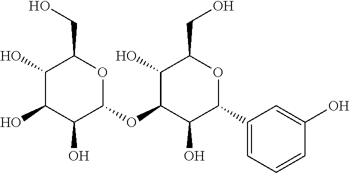 [SMILES: OC[C@H]1O[C@H](O[C@@H]2[C@H](O)[C@@H](c3cccc(O)c3)O[C@H](CO)[C@H]2O)[C@@H](O)[C@@H](O)[C@@H]1O] | 265 |
| 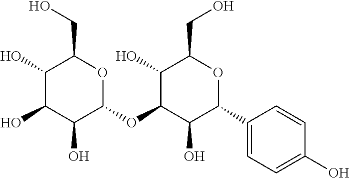 [SMILES: OC[C@H]1O[C@H](O[C@@H]2[C@H](O)[C@@H](c3ccc(O)cc3)O[C@H](CO)[C@H]2O)[C@@H](O)[C@@H](O)[C@@H]1O] | 266 |
| 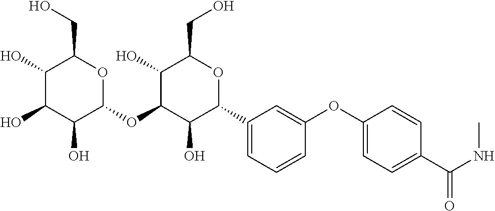 [SMILES: CNC(=O)c1ccc(Oc2cccc([C@H]3O[C@H](CO)[C@@H](O)[C@H](O[C@H]4O[C@H](CO)[C@@H](O)[C@H](O)[C@@H]4O)[C@@H]3O)c2)cc1] | 267 |
| 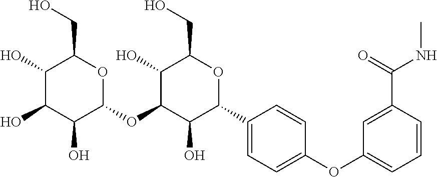 [SMILES: CNC(=O)c1cccc(Oc2ccc([C@H]3O[C@H](CO)[C@@H](O)[C@H](O[C@H]4O[C@H](CO)[C@@H](O)[C@H](O)[C@@H]4O)[C@@H]3O)cc2)c1] | 268 |
| 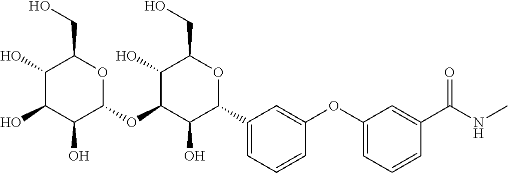 [SMILES: CNC(=O)c1cccc(Oc2cccc([C@H]3O[C@H](CO)[C@@H](O)[C@H](O[C@H]4O[C@H](CO)[C@@H](O)[C@H](O)[C@@H]4O)[C@@H]3O)c2)c1] | 269 |
| 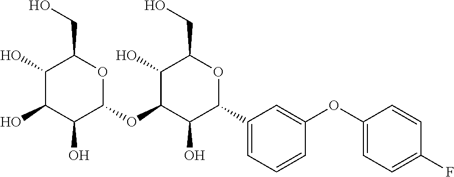 [SMILES: OC[C@H]1O[C@H](O[C@@H]2[C@H](O)[C@@H](c3cccc(Oc4ccc(F)cc4)c3)O[C@H](CO)[C@H]2O)[C@@H](O)[C@@H](O)[C@@H]1O] | 270 |
| 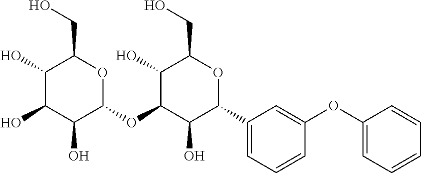 [SMILES: OC[C@H]1O[C@H](O[C@@H]2[C@H](O)[C@@H](c3cccc(Oc4ccccc4)c3)O[C@H](CO)[C@H]2O)[C@@H](O)[C@@H](O)[C@@H]1O] | 271 |
| 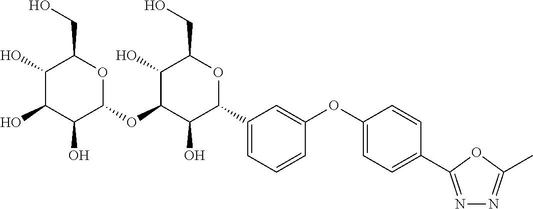 [SMILES: Cc1nnc(-c2ccc(Oc3cccc([C@H]4O[C@H](CO)[C@@H](O)[C@H](O[C@H]5O[C@H](CO)[C@@H](O)[C@H](O)[C@@H]5O)[C@@H]4O)c3)cc2)o1] | 272 |
| 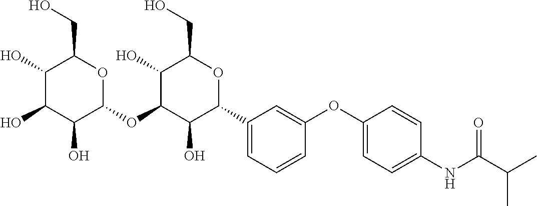 [SMILES: CC(C)C(=O)Nc1ccc(Oc2cccc([C@H]3O[C@H](CO)[C@@H](O)[C@H](O[C@H]4O[C@H](CO)[C@@H](O)[C@H](O)[C@@H]4O)[C@@H]3O)c2)cc1] | 273 |
| 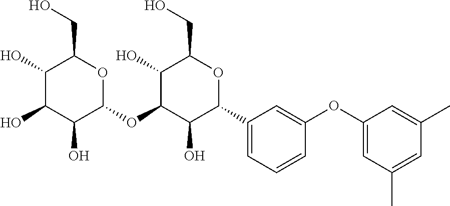 [SMILES: Cc1cc(C)cc(Oc2cccc([C@H]3O[C@H](CO)[C@@H](O)[C@H](O[C@H]4O[C@H](CO)[C@@H](O)[C@H](O)[C@@H]4O)[C@@H]3O)c2)c1] | 274 |
| 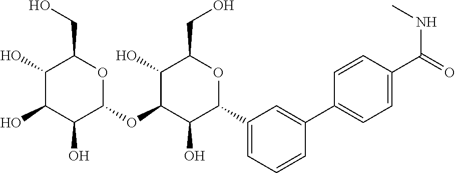 [SMILES: CNC(=O)c1ccc(-c2cccc([C@H]3O[C@H](CO)[C@@H](O)[C@H](O[C@H]4O[C@H](CO)[C@@H](O)[C@H](O)[C@@H]4O)[C@@H]3O)c2)cc1] | 275 |
| 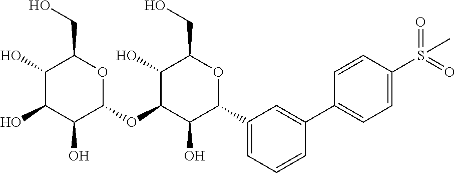 [SMILES: CS(=O)(=O)c1ccc(-c2cccc([C@H]3O[C@H](CO)[C@@H](O)[C@H](O[C@H]4O[C@H](CO)[C@@H](O)[C@H](O)[C@@H]4O)[C@@H]3O)c2)cc1] | 276 |
| 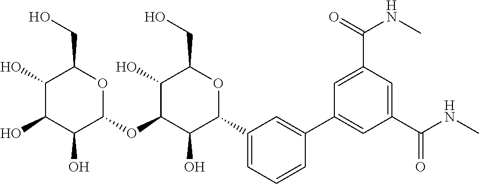 [SMILES: CNC(=O)c1cc(C(=O)NC)cc(-c2cccc([C@H]3O[C@H](CO)[C@@H](O)[C@H](O[C@H]4O[C@H](CO)[C@@H](O)[C@H](O)[C@@H]4O)[C@@H]3O)c2)c1] | 277 |
| 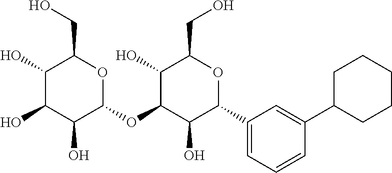 [SMILES: OC[C@H]1O[C@H](O[C@@H]2[C@H](O)[C@@H](c3cccc(C4CCCCC4)c3)O[C@H](CO)[C@H]2O)[C@@H](O)[C@@H](O)[C@@H]1O] | 278 |
| 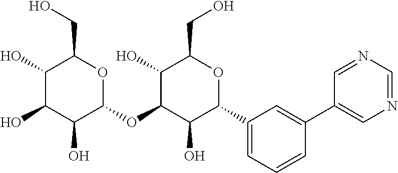 [SMILES: OC[C@H]1O[C@H](O[C@@H]2[C@H](O)[C@@H](c3cccc(-c4cncnc4)c3)O[C@H](CO)[C@H]2O)[C@@H](O)[C@@H](O)[C@@H]1O] | 279 |
| 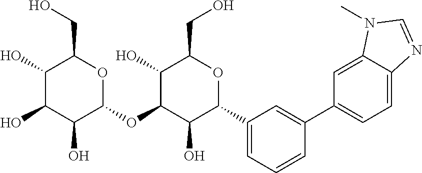 [SMILES: Cn1cnc2ccc(-c3cccc([C@H]4O[C@H](CO)[C@@H](O)[C@H](O[C@H]5O[C@H](CO)[C@@H](O)[C@H](O)[C@@H]5O)[C@@H]4O)c3)cc21] | 280 |
| 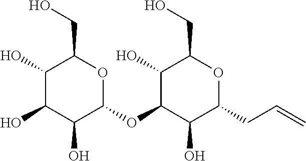 [SMILES: C=CC[C@H]1O[C@H](CO)[C@@H](O)[C@H](O[C@H]2O[C@H](CO)[C@@H](O)[C@H](O)[C@@H]2O)[C@@H]1O] | 281 |
| 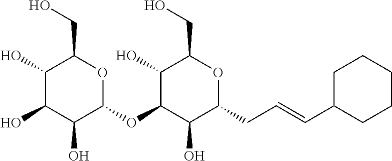 [SMILES: OC[C@H]1O[C@H](O[C@H]2[C@H](O)[C@@H](CO)O[C@H](C/C=C/C3CCCCC3)[C@H]2O)[C@@H](O)[C@@H](O)[C@@H]1O] | 282 |
| 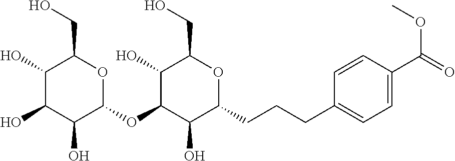 [SMILES: COC(=O)c1ccc(CCC[C@H]2O[C@H](CO)[C@@H](O)[C@H](O[C@H]3O[C@H](CO)[C@@H](O)[C@H](O)[C@@H]3O)[C@@H]2O)cc1] | 283 |
| 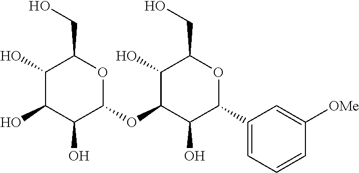 [SMILES: COc1cccc([C@H]2O[C@H](CO)[C@@H](O)[C@H](O[C@H]3O[C@H](CO)[C@@H](O)[C@H](O)[C@@H]3O)[C@@H]2O)c1] | 284 |
| 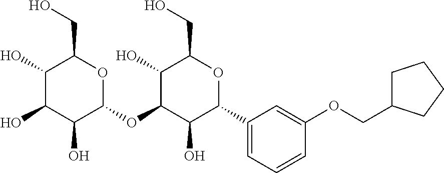 [SMILES: OC[C@H]1O[C@H](O[C@@H]2[C@H](O)[C@@H](c3cccc(OCC4CCCC4)c3)O[C@H](CO)[C@H]2O)[C@@H](O)[C@@H](O)[C@@H]1O] | 285 |
| 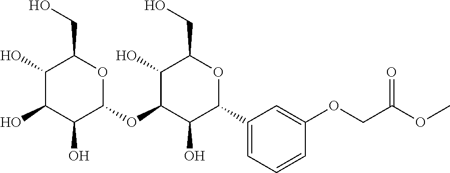 [SMILES: COC(=O)COc1cccc([C@H]2O[C@H](CO)[C@@H](O)[C@H](O[C@H]3O[C@H](CO)[C@@H](O)[C@H](O)[C@@H]3O)[C@@H]2O)c1] | 286 |
| 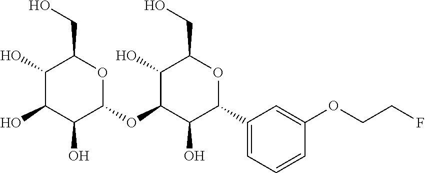 [SMILES: OC[C@H]1O[C@H](O[C@@H]2[C@H](O)[C@@H](c3cccc(OCCF)c3)O[C@H](CO)[C@H]2O)[C@@H](O)[C@@H](O)[C@@H]1O] | 287 |
| 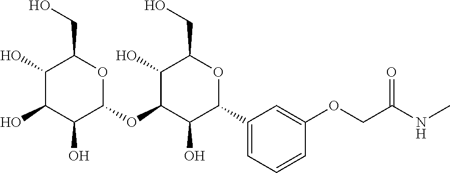 [SMILES: CNC(=O)COc1cccc([C@H]2O[C@H](CO)[C@@H](O)[C@H](O[C@H]3O[C@H](CO)[C@@H](O)[C@H](O)[C@@H]3O)[C@@H]2O)c1] | 288 |
| 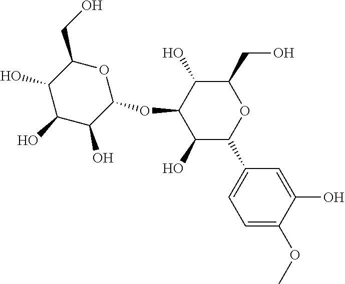 | 289 |
| 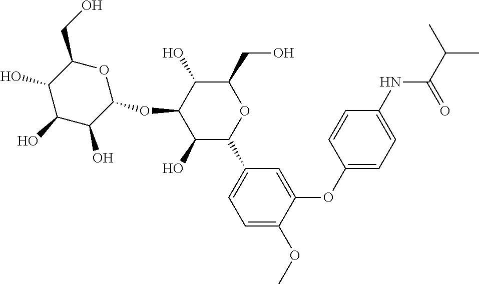 | 290 |
| 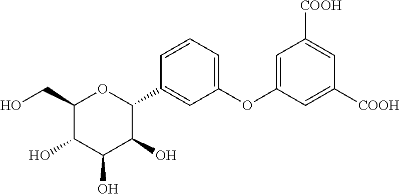 [SMILES: O=C(O)c1cc(Oc2cccc([C@H]3O[C@H](CO)[C@@H](O)[C@H](O)[C@@H]3O)c2)cc(C(=O)O)c1] | 291 |
| 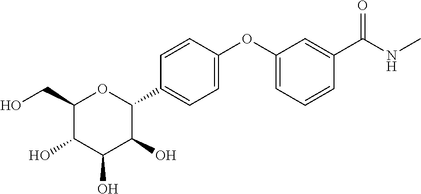 [SMILES: CNC(=O)c1cccc(Oc2ccc([C@H]3O[C@H](CO)[C@@H](O)[C@H](O)[C@@H]3O)cc2)c1] | 292 |
| 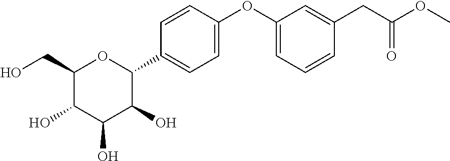 [SMILES: COC(=O)Cc1cccc(Oc2ccc([C@H]3O[C@H](CO)[C@@H](O)[C@H](O)[C@@H]3O)cc2)c1] | 293 |
| 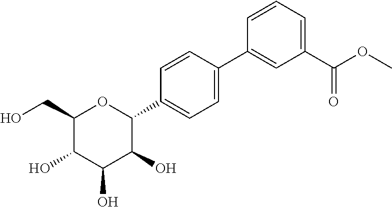 [SMILES: COC(=O)c1cccc(-c2ccc([C@H]3O[C@H](CO)[C@@H](O)[C@H](O)[C@@H]3O)cc2)c1] | 294 |
| 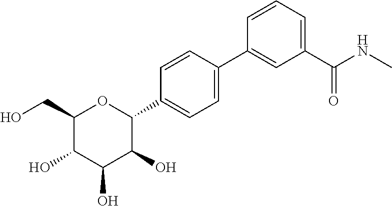 [SMILES: CNC(=O)c1cccc(-c2ccc([C@H]3O[C@H](CO)[C@@H](O)[C@H](O)[C@@H]3O)cc2)c1] | 295 |
| 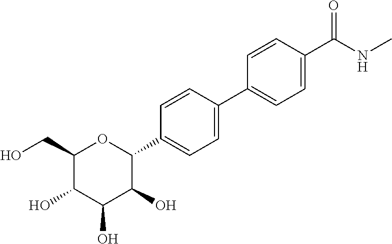 [SMILES: CNC(=O)c1ccc(-c2ccc([C@H]3O[C@H](CO)[C@@H](O)[C@H](O)[C@@H]3O)cc2)cc1] | 296 |

Another embodiment provides a compound selected from one or more of the following: Compound 48, 104, 105, 106, 107, 108, 111, 112, 120, 121, 125, 126, 127, 128, 131, 133, 136, 142, 150, 176, or 178. Another embodiment provides a compound selected from the group consisting of Compound 265 to Compound 290. Yet another embodiment provides a compound selected from the group consisting of Compound 1 to Compound 72 and Compound 291 to compound 296.

For the sake of clarity, it shall be understood that a list of sequential compounds includes all compounds within the range. For example, “Compound 1 to Compound 3” also means Compound 1, Compound 2, and Compound 3.

### Processes

The present invention also provides processes for making compounds of the invention.

One embodiment provides a process for making Compound 48:

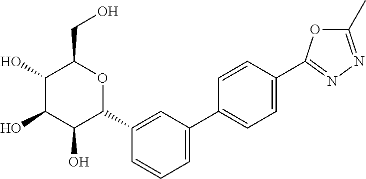

Comprising one or more of the following steps:
A) Reacting a compound of formula i-a:

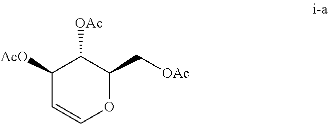 [SMILES: CC(=O)OC[C@H]1OC=C[C@@H](OC(C)=O)[C@@H]1OC(C)=O]

with a compound of formula i-b:

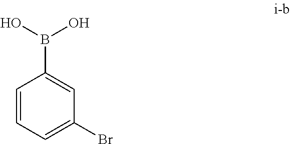

under suitable Suzuki coupling conditions known to one of skill in the art (e.g., an appropriate palladium coupling agent in a appropriate solvent, such as diacetoxypalladium in acetonitrile) to form a compound of formula i-c:

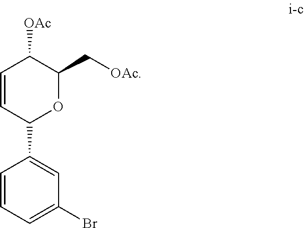

B) Reacting the compound of formula i-c under suitable hydroxylation conditions (e.g., OsO₄ and 4-methyl-4-oxido-morpholin-4-ium in tetrahydrofuran) to form a compound of formula i-d:

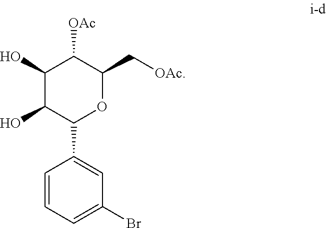

C) Reacting a compound of formula i-d under suitable acetylation conditions (e.g., acetic anhydride, a suitable base (such as dimethylaminopyridine), and a suitable solvent (such as pyridine) to form a compound of formula i-e:

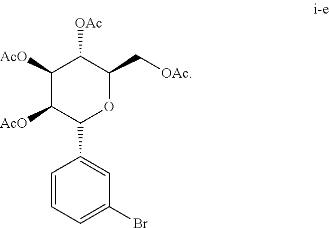

D) Reacting a compound of formula i-e with a compound of formula i-f:

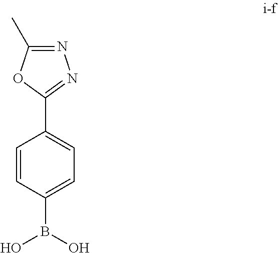

under suitable Suzuki coupling conditions known to one of skill in the art (e.g., an appropriate palladium coupling agent with an optional base in a appropriate solvent, such as diacetoxypalladium in acetonitrile or Pd(PPh₃)₄ with sodium bicarbonate in dioxane) to form a compound of formula i-g:

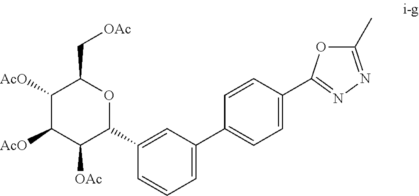

E) Deprotecting the compound of formula i-g under suitable acetyl-removal conditions (e.g., a strong base in a suitable solvent, such as MeONa in methanol) to form Compound 48.

The present invention also provides a composition comprising the compound described herein, and a pharmaceutically acceptable carrier, adjuvant, or vehicle.

The present invention also provides a method of treating or preventing bacteria infection in a subject, comprising administering to the subject an effective amount of the compound or the composition described herein.

In an embodiment of the method, the bacteria infection is urinary tract infection or inflammatory bowel disease.

As described herein, a specified number range of atoms includes any integer therein. For example, a group having from 1-4 atoms could have 1, 2, 3, or 4 atoms.

The term “stable”, as used herein, refers to compounds that are not substantially altered when subjected to conditions to allow for their production, detection, recovery, storage, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched), or branched, hydrocarbon chain that is completely saturated or that contains one or more units of unsaturation but is non-aromatic.

Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. Aliphatic groups may be linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl.

The term “alkyl” as used herein means a saturated straight or branched chain hydrocarbon. The term “alkenyl” as used herein means a straight or branched chain hydrocarbon comprising one or more double bonds. The term “alkynyl” as used herein means a straight or branched chain hydrocarbon comprising one or more triple bonds.

The term “cycloaliphatic” (or “carbocycle” or “carbocyclyl” or “carbocyclic”) refers to a non-aromatic monocyclic carbon containing ring which can be saturated or contain one or more units of unsaturation, having three to fourteen ring carbon atoms. In some embodiments, the ring has three to ten ring carbon atoms; in other embodiments, the ring has three to six carbon atoms. The term includes polycyclic fused, spiro or bridged carbocyclic ring systems. The term also includes polycyclic ring systems in which the carbocyclic ring can be fused to one or more non-aromatic carbocyclic or heterocyclic rings or one or more aromatic rings or combination thereof, wherein the radical or point of attachment is on the carbocyclic ring. Fused bicyclic ring systems comprise two rings which share two adjoining ring atoms, bridged bicyclic group comprise two rings which share three or four adjacent ring atoms, spiro bicyclic ring systems share one ring atom. Examples of cycloaliphatic groups include, but are not limited to, cycloalkyl and cycloalkenyl groups. Specific examples include, but are not limited to, cyclohexyl, cyclopropenyl, and cyclobutyl.

The term “heterocycle” (or “heterocyclyl”, or “heterocyclic”) as used herein means refers to a non-aromatic monocyclic ring which can be saturated or contain one or more units of unsaturation, having three to fourteen ring atoms in which one or more ring carbons is replaced by a heteroatom such as, N, S, or O. In some embodiments, the ring has three to ten ring atoms; in other embodiments, the ring has three to six ring atoms. In yet other embodiments, the ring has five to six ring atoms. The term includes polycyclic fused, spiro or bridged heterocyclic ring systems. The term also includes polycyclic ring systems in which the heterocyclic ring can be fused to one or more non-aromatic carbocyclic or heterocyclic rings or one or more aromatic rings or combination thereof, wherein the radical or point of attachment is on the heterocyclic ring.

Examples of heterocycles include, but are not limited to, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydropyranyl, tetrahydrothiophenyl, morpholino, including, for example, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, thienothienyl, thienothiazolyl, benzothiolanyl, benzodithianyl, 3-(1-alkyl)-benzimidazol-2-onyl, and 1,3-dihydro-imidazol-2-onyl.

Cyclic groups, (e.g. cycloaliphatic and heterocycles), can be linearly fused, bridged, or spirocyclic.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation. As would be known by one of skill in the art, unsaturated groups can be partially unsaturated or fully unsaturated. Examples of partially unsaturated groups include, but are not limited to, butene, cyclohexene, and tetrahydropyridine. Fully unsaturated groups can be aromatic, anti-aromatic, or non-aromatic. Examples of fully unsaturated groups include, but are not limited to, phenyl, cyclooctatetraene, pyridyl, thienyl, and 1-methylpyridin-2(1H)-one.

The term “alkoxy”, or “thioalkyl”, as used herein, refers to an alkyl group, as previously defined, attached to the molecule through an oxygen (“alkoxy” e.g., —O-alkyl) or sulfur (“thioalkyl” e.g., —S-alkyl) atom.

The terms “haloalkyl”, “haloalkenyl”, “haloaliphatic”, and “haloalkoxy” mean alkyl, alkenyl or alkoxy, as the case may be, substituted with one or more halogen atoms. This term includes perfluorinated alkyl groups, such as —CF₃ and —CF₂CF₃.

The terms “halogen”, “halo”, and “hal” mean F, Cl, Br, or I.

The term “aryl” used alone or as part of a larger moiety as in “aralkyl”, “aralkoxy”, or “aryloxyalkyl”, refers to carbocyclic aromatic ring systems. The term “aryl” may be used interchangeably with the term “aryl ring”.

Carbocyclic aromatic ring groups have only carbon ring atoms (typically six to fourteen) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring”, as it is used herein, is a group in which an aromatic ring is fused to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring.

The term “heteroaryl”, “heteroaromatic”, “heteroaryl ring”, “heteroaryl group” and “heteroaromatic group”, used alone or as part of a larger moiety as in “heteroaralkyl” or “heteroarylalkoxy”, refers to heteroaromatic ring groups having five to fourteen members, including monocyclic heteroaromatic rings and polycyclic aromatic rings in which a monocyclic aromatic ring is fused to one or more other aromatic ring. Heteroaryl groups have one or more ring heteroatoms. Also included within the scope of the term “heteroaryl”, as it is used herein, is a group in which an aromatic ring is fused to one or more non-aromatic rings (carbocyclic or heterocyclic), where the radical or point of attachment is on the aromatic ring. Bicyclic 6,5 heteroaromatic ring, as used herein, for example, is a six membered heteroaromatic ring fused to a second five membered ring, wherein the radical or point of attachment is on the six membered ring.

It shall be understood that a 5-10 membered heteroaryl includes both monocyclic and bicyclic rings. For example, it could include 5-6 membered monocyclic rings having 1-4 heteroatoms selected from oxygen, nitrogen, or sulfur and 8-10 membered bicyclic rings having 1-6 heteroatoms selected from oxygen, nitrogen, or sulfur.

Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, benzotriazolyl, benzothiazolyl, benzoxazolyl, benzimidazolyl, isoquinolinyl, indolyl, isoindolyl, acridinyl, benzisoxazolyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “protecting group” and “protective group” as used herein, are interchangeable and refer to an agent used to temporarily block one or more desired functional groups in a compound with multiple reactive sites. In certain embodiments, a protecting group has one or more, or preferably all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

In some embodiments, where indicated, a methylene unit of an aliphatic chain is optionally replaced with another atom or group. Examples of such atoms or groups include, but are not limited to, —NR—, —O—, —C(O)—, —C(═N—CN)—, —C(═NR)—, —C(═NOR)—, —S—, —S(O)—, and —S(O)₂—. These atoms or groups can be combined to form larger groups. Examples of such larger groups include, but are not limited to, —OC(O)—, —C(O)CO—, —CO₂—, —C(O)NR—, —C(═N—CN), —NRC(O)—, —NRC(O)O—, —S(O)₂NR—, —NRSO₂—, —NRC(O)NR—, —OC(O)NR—, and —NRSO₂NR—, wherein R is for example, H or C₁₋₆aliphatic, or is otherwise defined herein.

It should be understood that these groups can be bonded to the methylene units of the aliphatic chain via single, double, or triple bonds. An example of an optional replacement (nitrogen atom in this case) that is bonded to the aliphatic chain via a double bond would be —CH₂CH═N—CH₃. In some cases, especially on the terminal end, an optional replacement can be bonded to the aliphatic group via a triple bond. One example of this would be CH₂CH₂CH₂C≡N. It should be understood that in this situation, the terminal nitrogen is not bonded to another atom.

It should also be understood that, the term “methylene unit” can also refer to branched or substituted methylene units. For example, in an isopropyl moiety [—CH(CH₃)₂], a nitrogen atom (e.g. NR) replacing the first recited “methylene unit” would result in dimethylamine [—N(CH₃)₂]. In instances such as these, one of skill in the art would understand that the nitrogen atom will not have any additional atoms bonded to it, and the “R” from “NR” would be absent in this case.

Only those replacement and combinations of groups that result in a stable structure are contemplated. Optional replacements can occur both within the chain and/or at either end of the chain; i.e. both at the point of attachment and/or also at the terminal end. Two optional replacements can also be adjacent to each other within a chain so long as it results in a chemically stable compound. The optional replacements can also completely replace all of the carbon atoms in a chain. For example, a C₃ aliphatic can be optionally replaced by —NR—, —C(O)—, and —NR— to form —NRC(O)NR— (a urea).

Unless otherwise indicated, if the replacement occurs at the terminal end, the replacement atom is bound to an H on the terminal end. For example, if —CH₂CH₂CH₃ were optionally replaced with —O—, the resulting compound could be —OCH₂CH₃, —CH₂OCH₃, or —CH₂CH₂OH. It should be understood that if the terminal atom does not contain any free valence electrons, then a hydrogen atom is not required at the terminal end (e.g., —CH₂CH₂CH═O or —CH₂CH₂C≡N).

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. For example, a substituent drawn as

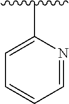 [SMILES: *c1ccccn1]

also represents

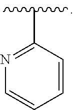 [SMILES: *c1ccccn1]

Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

“D” and “d” both refer to deuterium.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays.

As described herein, where indicated compounds of the invention may optionally be substituted with one or more substituents, such as are illustrated generally herein, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position.

Only those choices and combinations of substituents that result in a stable structure are contemplated. Such choices and combinations will be apparent to those of ordinary skill in the art and may be determined without undue experimentation.

The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen.

An aryl group as defined herein may contain one or more substitutable ring atoms, which may be bonded to a suitable substituent. Examples of suitable substituents on a substitutable ring carbon atom of an aryl group include R′. R′ is —Ra, —Br, —Cl, —I, —F, —ORa, —SRa, —O—CORa, —CORa, —CSRa, —CN, —NO₂, —NCS, —SO₃H, —N(RaRb), —COORa, —NRcNRcCORa, —NRcNRcCO₂Ra, —CHO, —CON(RaRb), —OC(O)N(RaRb), —CSN(RaRb), —NRcCORa, —NRcCOORa, —NRcCSRa, —NRcCON(RaRb), —NRcNRcC(O)N(RaRb), —NRcCSN(RaRb), —C(═NRc)-N(RaRb), —C(═S)N(RaRb), —NRd-C(═NRc)-N(RaRb), —NRcNRaRb, —S(O)ₚNRaRb, —NRcSO₂N(RaRb), —NRcS(O)ₚRa, —S(O)ₚRa, —OS(O)ₚNRaRb or —OS(O)ₚRa; wherein p is 1 or 2.

Ra-Rd are each independently —H, an aliphatic group, aromatic group, non-aromatic carbocyclic or heterocyclic group or —N(RaRb), taken together, form a non-aromatic heterocyclic group. The aliphatic, aromatic and non-aromatic heterocyclic group represented by Ra-Rd and the non-aromatic heterocyclic group represented by —N(RaRb) are each optionally and independently substituted with one or more groups represented by R^{#}. Preferably Ra-Rd are unsubstituted.

R^{#} is halogen, R⁺, —OR⁺, —SR⁺, —NO₂, —CN, —N(R⁺)₂, —COR⁺, —COOR⁺, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)N(R⁺)₂, —OC(O)R⁺, —OC(O)N(R⁺)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂.

R⁺ is —H, a C1-C4 alkyl group, a monocyclic aryl group, a non-aromatic carbocyclic or heterocyclic group each optionally substituted with alkyl, haloalkyl, alkoxy, haloalkoxy, halo, —CN, —NO₂, amine, alkylamine or dialkylamine Preferably R+ is unsubstituted.

An aliphatic or a non-aromatic heterocyclic or carbocyclic group as used herein may contain one or more substituents. Examples of suitable substituents for an aliphatic group or a ring carbon of a non-aromatic heterocyclic group is R″. R″ include those substituents listed above for R′ and ═O, ═S, ═NNHR**, ═NN(R**)2, ═NNHC(O)R**, ═NNHCO2 (alkyl), ═NNHSO2 (alkyl), ═NR**, Spiro cycloalkyl group or fused cycloalkyl group. Each R** is independently selected from hydrogen, an unsubstituted alkyl group or a substituted alkyl group. Examples of substituents on the alkyl group represented by R** include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

A preferred position for substitution of a non-aromatic nitrogen-containing heterocyclic group is the nitrogen ring atom. Suitable substituents on the nitrogen of a non-aromatic heterocyclic group or heteroaryl group include —R̂, —N(R̂)₂, C(O)R̂, CO₂R̂, —C(O)C(O)R̂, —SO₂R̂, SO₂ N(R̂)₂, C(═S)N(R̂)₂, C(═NH)—N(R̂)₂, and —NR̂SO₂R̂; wherein R̂ is hydrogen, an aliphatic group, a substituted aliphatic group, aryl, substituted aryl, heterocyclic or carbocyclic ring or a substituted heterocyclic or carbocyclic ring. Examples of substituents on the group represented by R̂ include alkyl, haloalkoxy, haloalkyl, alkoxyalkyl, sulfonyl, alkylsulfonyl, halogen, nitro, cyano, hydroxy, aryl, carbocyclic or heterocyclic ring, oxo, amino, alkylamino, dialkylamino, aminocarbonyl, alkylaminocarbonyl, dialkylaminocarbonyloxy, alkoxy, carboxy, alkoxycarbonyl, or alkylcarbonyl. Preferably R̂ is not substituted.

Non-aromatic nitrogen containing heterocyclic rings that are substituted on a ring nitrogen and attached to the remainder of the molecule at a ring carbon atom are said to be N substituted. For example, an N alkyl piperidinyl group is attached to the remainder of the molecule at the two, three or four position of the piperidinyl ring and substituted at the ring nitrogen with an alkyl group. Non-aromatic nitrogen containing heterocyclic rings such as pyrazinyl that are substituted on a ring nitrogen and attached to the remainder of the molecule at a second ring nitrogen atom are said to be N′ substituted-N-heterocycles. For example, an N′ acyl N-pyrazinyl group is attached to the remainder of the molecule at one ring nitrogen atom and substituted at the second ring nitrogen atom with an acyl group.

As used herein an optionally substituted aralkyl can be substituted on both the alkyl and the aryl portion. Unless otherwise indicated as used herein optionally substituted aralkyl is optionally substituted on the aryl portion.

The terms “a bond” and “absent” are used interchangeably to indicate that a group is absent.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds of this invention can exist in free form for treatment, or where appropriate, as a pharmaceutically acceptable salt.

### Solid Forms

Another aspect of this invention provides solid forms of the compounds of this invention. On embodiment provides a solid form of compound 48 wherein the form is selected from the group consisting of Compound 48 free base.

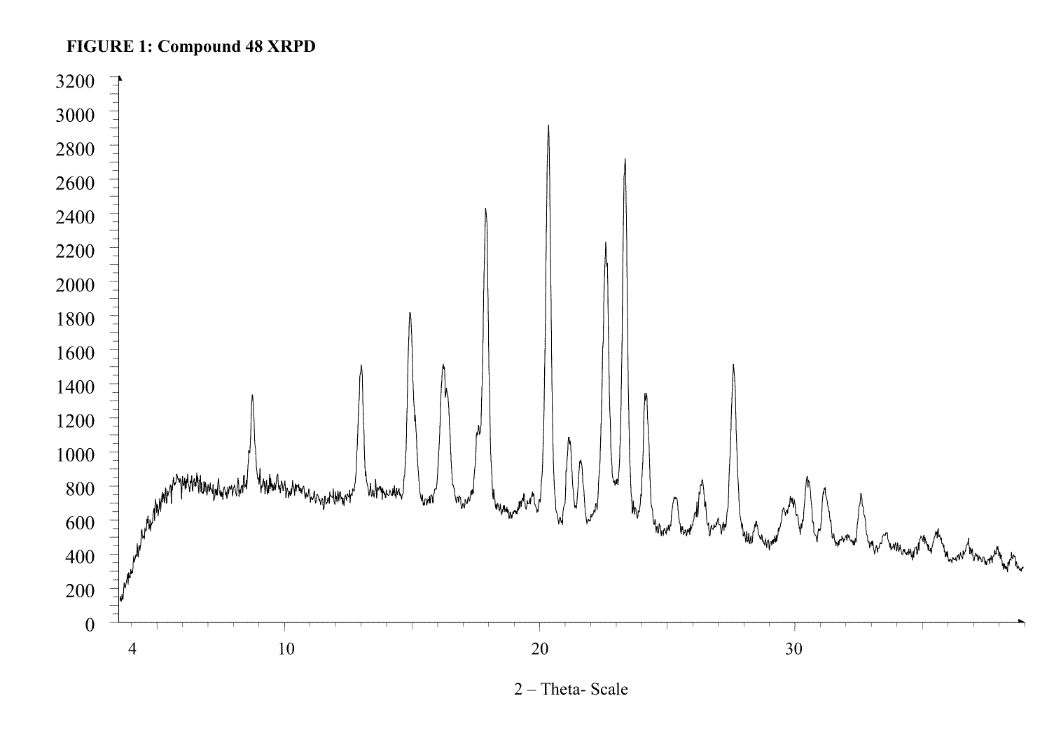

In some embodiments, Compound 48 free base is characterized by a weight loss of from about XX in a temperature range of from about 25° C. to about 350° C. In other embodiments, Compound 48 free base is characterized by one or more peaks expressed in 2-theta±0.2 at 4°-45° in a X-ray powder diffraction pattern obtained using Cu K alpha radiation. In yet other embodiments, crystalline Compound 48 free base is characterized by one or more peaks expressed in 2-theta±0.2 at the values described in the peak chart herein. In some embodiments, crystalline Compound 48 free base is characterized by having an X-ray powder diffraction pattern substantially the same as that shown in FIG. 1.

### Pharmaceutically Acceptable Salts

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

It should be understood that this invention includes mixtures/combinations of different pharmaceutically acceptable salts and also mixtures/combinations of compounds in free form and pharmaceutically acceptable salts.

In addition to the compounds of this invention, pharmaceutically acceptable derivatives or prodrugs of the compounds of this invention may also be employed in compositions to treat or prevent the herein identified disorders.

As used herein and unless otherwise indicated, the term “prodrug” means a derivative of a compound that can hydrolyze, oxidize, or otherwise react under biological conditions (in vitro or in vivo) to provide a compound of this invention. Prodrugs may become active upon such reaction under biological conditions, or they may have activity in their unreacted forms. Examples of prodrugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of the invention that comprise OH moieties. Prodrugs can typically be prepared using well-known methods, such as those described by BURGER'S MEDICINAL CHEMISTRY AND DRUG DISCOVERY (1995) 172-178, 949-982 (Manfred E. Wolff ed., 5th ed).

A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

A “pharmaceutically acceptable derivative or prodrug” includes any pharmaceutically acceptable ester, salt of an ester or other derivative or salt thereof of a compound, of this invention which, upon administration to a recipient, is capable of providing, either directly or indirectly, a compound of this invention or an inhibitorily active metabolite or residue thereof. Particularly favoured derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Pharmaceutically acceptable prodrugs of the compounds of this invention include, without limitation, esters, amino acid esters, phosphate esters, metal salts and sulfonate esters.

As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

In one embodiment the present invention is a pharmaceutical composition comprising a compound of the present invention and a pharmaceutically acceptable carrier, diluent, adjuvant or vehicle. In one embodiment the present invention is a pharmaceutical composition comprising an effective amount of compound of the present invention and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds of present invention or pharmaceutical salts thereof may be formulated into pharmaceutical compositions for administration to a subject as defined herein. These pharmaceutical compositions, which comprise an amount of the compounds effective to treat or prevent a bacteria infection, such as IBD, and a pharmaceutically acceptable carrier, are another embodiment of the present invention.

In one embodiment the present invention is a method of treating or preventing a bacteria infection, such as IBD, in a subject in need thereof, comprising administering to the subject an effective amount of a compound or composition of the present invention.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, an “effective amount” refers to an amount sufficient to elicit the desired biological response. In the present invention the desired biological response is to reduce or ameliorate the severity, duration, progression, or onset of a bateria infection, prevent the advancement of a bateria infection, cause the regression of a bateria infection, prevent the recurrence, development, onset or progression of a symptom associated with a bateria infection, or enhance or improve the prophylactic or therapeutic effect(s) of another therapy. The precise amount of compound administered to a subject will depend on the mode of administration, the type and severity of the disease or condition and on the characteristics of the subject, such as general health, age, sex, body weight and tolerance to drugs. It will also depend on the degree, severity and type of bateria infection, and the mode of administration. The skilled artisan will be able to determine appropriate dosages depending on these and other factors. When co-administered with other agents, e.g., when co-administered with a bateria infection agent, an “effective amount” of the second agent will depend on the type of drug used. Suitable dosages are known for approved agents and can be adjusted by the skilled artisan according to the condition of the subject, the type of condition(s) being treated and the amount of a compound of the invention being used. In cases where no amount is expressly noted, an effective amount should be assumed.

As used herein, the terms “treat”, “treatment” and “treating” refer to the reduction or amelioration of the progression, severity and/or duration of a bateria infection, or the amelioration of one or more symptoms (preferably, one or more discernible symptoms) of a bateria infection resulting from the administration of one or more therapies (e.g., one or more therapeutic agents such as a compound of the invention). In specific embodiments, the terms “treat”, “treatment” and “treating” refer to the amelioration of at least one measurable physical parameter of a bacteria infection. In other embodiments the terms “treat”, “treatment” and “treating” refer to the inhibition of the progression of a bateria infection, either physically by, e.g., stabilization of a discernible symptom, physiologically by, e.g., stabilization of a physical parameter, or both. In other embodiments the terms “treat”, “treatment” and “treating” refer to the reduction or stabilization of a bateria infection.

As used herein, the terms “prevent”, “prevention” and “preventing” refer to the reduction in the risk of acquiring or developing a given bateria infection, or the reduction or inhibition of the recurrence or a bateria infection. In one embodiment, a compound of the invention is administered as a preventative measure to a patient, preferably a human, having a genetic predisposition to any of the conditions, diseases or disorders described herein.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like. The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions of the present invention may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.
The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.
Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The dosage regimen utilizing the compounds of present invention can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compound of present invention required to treat, for example, to prevent, inhibit (fully or partially) or arrest the progress of the disease.

Dosages of the compounds of present invention can range from between about 0.01 to about 100 mg/kg body weight/day, about 0.01 to about 50 mg/kg body weight/day, about 0.1 to about 50 mg/kg body weight/day, or about 1 to about 25 mg/kg body weight/day. It is understood that the total amount per day can be administered in a single dose or can be administered in multiple dosings such as twice, three or four times per day.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

An effective amount can be achieved in the method or pharmaceutical composition of the invention employing a compound of present invention or a pharmaceutically acceptable salt thereof alone or in combination with an additional suitable therapeutic agent, for example, a cancer-therapeutic agent. When combination therapy is employed, an effective amount can be achieved using a first amount of a compound of present invention or a pharmaceutically acceptable salt thereof and a second amount of an additional suitable therapeutic agent.

In one embodiment, the compound of present invention and the additional therapeutic agent, are each administered in an effective amount (i.e., each in an amount which would be therapeutically effective if administered alone). In another embodiment, the compound of present invention and the additional therapeutic agent, are each administered in an amount which alone does not provide a therapeutic effect (a sub-therapeutic dose). In yet another embodiment, the compound of present invention can be administered in an effective amount, while the additional therapeutic agent is administered in a sub-therapeutic dose. In still another embodiment, the compound of present invention can be administered in a sub-therapeutic dose, while the additional therapeutic agent, for example, a suitable cancer-therapeutic agent is administered in an effective amount.

As used herein, the terms “in combination” or “coadministration” can be used interchangeably to refer to the use of more than one therapies (e.g., one or more prophylactic and/or therapeutic agents). The use of the terms does not restrict the order in which therapies (e.g., prophylactic and/or therapeutic agents) are administered to a subject. Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order.

When coadministration involves the separate administration of the first amount of a compound of present invention and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of present invention and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-cancer agent) to a subject.

It is understood that the method of coadministration of a first amount of a compound of present invention and a second amount of an additional therapeutic agent can result in an enhanced or synergistic therapeutic effect, wherein the combined effect is greater than the additive effect that would result from separate administration of the first amount of the compound of present invention and the second amount of the additional therapeutic agent.

As used herein, the term “synergistic” refers to a combination of a compound of the invention and another therapy (e.g., a prophylactic or therapeutic agent), which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

The activity of the compounds as inhibitors of bacteria infection may be assayed in vitro or in vivo. In vitro assays include assays that determine inhibition of the FimH activity. Alternate in vitro assays quantitate the ability of the inhibitor to bind to the FimH and may be measured either by radiolabelling the inhibitor prior to binding, isolating the inhibitor complex and determining the amount of radiolabel bound, or by running a competition experiment where new inhibitors are incubated with the FimH bound to known radioligands. Detailed conditions for assaying a compound utilized in this invention are set forth in the Examples below.

### Experimental Details

The following abbreviations are used in the examples below:
AcOH acetic acid
AC₂O acetic anhydride
aq aqueous
BF₃.OEt₂ diethyloxonio-trifluoro-boron
CH₃CN acetonitrile
CCl₃CN trichloroacetonitrile
CDCl₃ chloroform-D
conc concentrate
CV column volume
Cs₂CO₃ cesium carbonate
Cu(OAc)₂ diacetoxycopper
DCM methylene chloride or dichloromethane
DMAP 4-dimethylaminopyridine
DMF dimethylformamide
DMSO dimethylsulfoxide
Eq. equivalent
EtOAc ethyl acetate
h hour
Hex hexanes
LiOH.H₂O lithium hydroxide monohydrate
M molar
MeOH methanol
MeONa sodium methoxide
Min minute
MS 4 Å molecular sieves 4 angstrom
MTBE methyl tert-butyl ether
Na₂SO₄ sodium sulfate

### NMO N-methylmorpholine-N-oxide

OsO₄ osmium tetroxide
PdCl₂ palladium (II)chloride
Pd(OAc)₂ palladium (II)acetate
PdCl₂(dppf)₂.CH₂Cl₂ (1,1′-Bis-(diphenylphosphino)-ferrocene)palladium (II) dichloride
Pd(OH)₂ dihydroxy palladium
Pd(PPh₃)₄ tetrakis(triphenylphosphine) palladium
Py pyridine
rt room temperature
Rt retention time
Siliacat DPP-Pd Silica supported diphenylphosphine palladium
TEA triethylamine
THF tetrahydrofuran
TLC thin layer chromatography
TMSOTf trimethylsilyl trifluoromethanesulfonate

The compounds of this invention may be prepared in light of the specification using steps generally known to those of ordinary skill in the art. Those compounds may be analyzed by known methods, including but not limited to LC-MS (liquid chromatography mass spectrometry), HPLC (high performance liquid chromatography) and NMR (nuclear magnetic resonance). It should be understood that the specific conditions shown below are only examples, and are not meant to limit the scope of the conditions that can be used for making compounds of this invention. Instead, this invention also includes conditions that would be apparent to those skilled in that art in light of this specification for making the compounds of this invention. Unless otherwise indicated, all variables in the following schemes are as defined herein.

Mass spec. samples are analyzed on a Waters UPLC Acquity mass spectrometer operated in single MS mode with electrospray ionization. Samples are introduced into the mass spectrometer using chromatography. Mobile phase for the mass spec. analyses consisted of 0.1% formic acid and acetonitrile-water mixture. Column gradient conditions are 5%-85% acetonitrile-water over 6 minutes run time Acquity HSS T3 1.8 um 2.1 mm ID×5 0 mm. Flow rate is 1.0 mL/min. As used herein, the term “Rt(min)” refers to the LC-MS retention time, in minutes, associated with the compound. Unless otherwise indicated, the LC-MS method utilized to obtain the reported retention time is as detailed above.

Purification by reverse phase HPLC is carried out under standard conditions using a Phenomenex Gemini 21.2 mm ID×250 mm column, 5 μm, 110 Å. Elution is performed using a linear gradient CH₃CN—H₂O (with or without 0.01% TFA buffer) as mobile phase. Solvent system is tailored according to the polarity of the compound, Flow rate, 20 mL/min. Compounds are collected either by UV or Waters 3100 Mass Detector, ESI Positive Mode. Fractions containing the desired compound are combined, concentrated (rotary evaporator) to remove excess CH3CN and the resulting aqueous solution is lyophilized to afford the desired material in most cases as a white foam.

HPLC analytical method is performed on Phenomenex Gemini C18 3 um 110 Å 4.6 mm ID×250 mm, Phenomenex Gemini C18 3 um 110 Å 4.6 mm ID×50 mm, using different combinations of CH₃CN—H₂O (0.01% TFA as buffer) as mobile phase, Flow rate, 1 mL/min, PDA 210 nm. Method A: Phenomenex Gemini C18 3 um 110 A 4.6 mm ID×250 mm; (10-50% acetonitrile-water for 40 min, 0.01% TFA). Method B: Phenomenex Gemini C18 3 um 110 A 4.6 mm ID×250 mm; (50-90% acetonitrile-water for 40 min, 0.01% TFA). Method C: Phenomenex Gemini C18 3 um 110 A 4.6 mm ID×50 mm; (20-60% acetonitrile-water for 10 min, 0.01% TFA). Method D: Phenomenex Gemini C18 3 um 110 A 4.6 mm ID×50 mm; (10-50% acetonitrile-water for 10 min, 0.01% TFA).

### Preparation of Compounds

The compounds of the invention may be made according to Scheme 1 below. The compounds may also be made according to the preparations described in the experimentals herein.

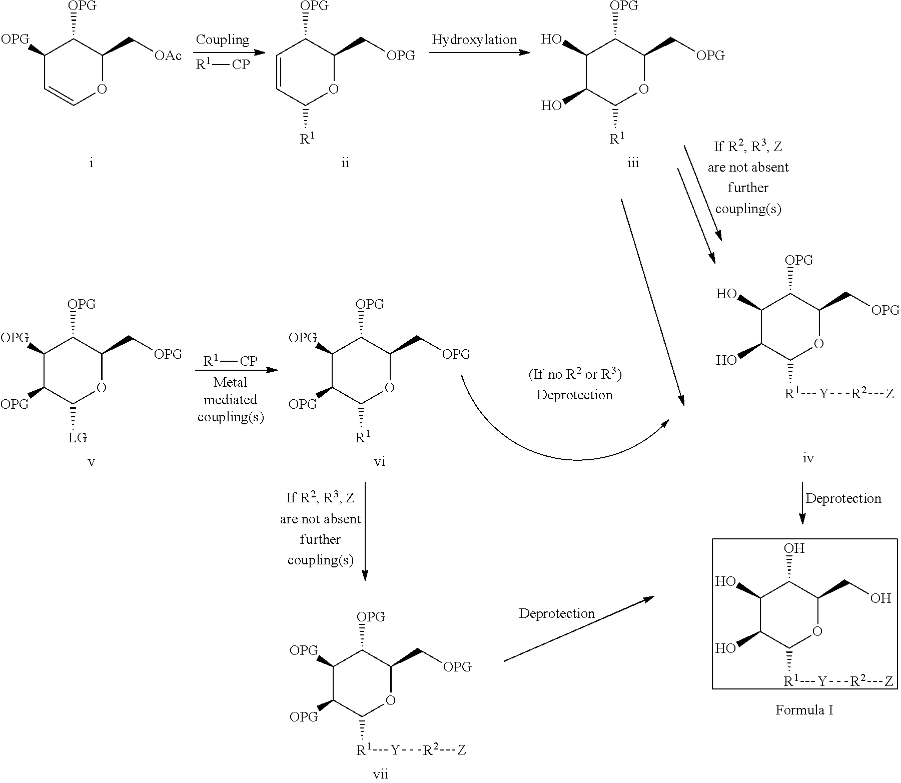

PG is a protecting group such as pivaloyl, acetyl, or other protecting groups known to one of skill in the art for protecting a hydroxyl group. CP is the appropriate coupling partner used in known metal mediated reactions such as, but not limited to, Sonagashira, Negishi, Suzuki, Stille couplings, and Goldberg reactions.

The starting dihydropyran i is coupled to an appropriate coupling partner R¹-CP (e.g., CP is a boronic acid) under suitable coupling conditions to form ii, which is then subject to appropriate hydroxylation conditions (e.g., OsO₄) to form tetrahydropyran iii. Tetrahydropyran iii can optionally be functionalized with a variety of groups using reactions such metal mediated couplings and other reactions known to one of skill in the art to form iv, which can then be deprotected under known deprotection conditions to form a compound of formula I.

Alternatively, protected tetrahydropyran v can be used, which can undergo similar coupling to an appropriate coupling partner R¹-CP (e.g., CP is a boronic acid) under suitable coupling conditions to form vi. Tetrahydropyran vi can optionally be functionalized with a variety of groups using reactions such metal mediated couplings and other reactions known to one of skill in the art to form vii, which can then be deprotected under known deprotection conditions to form a compound of formula I.

The following is a list of key INTERMEDIATES which are used in the preparation of Compounds.

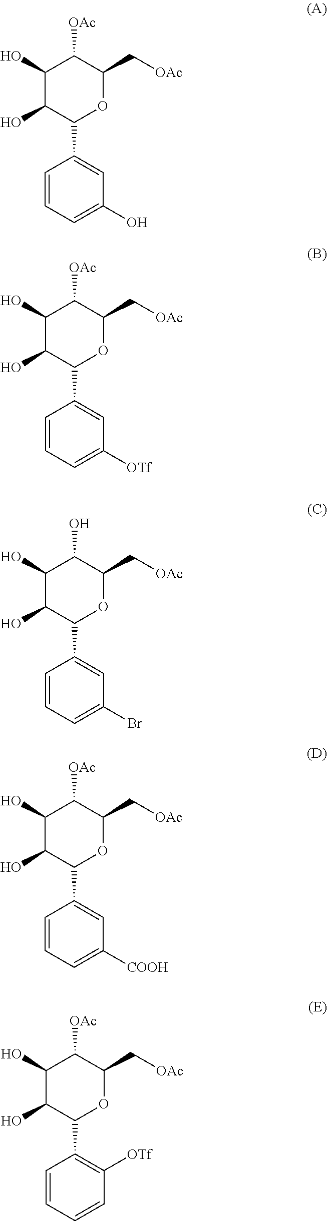

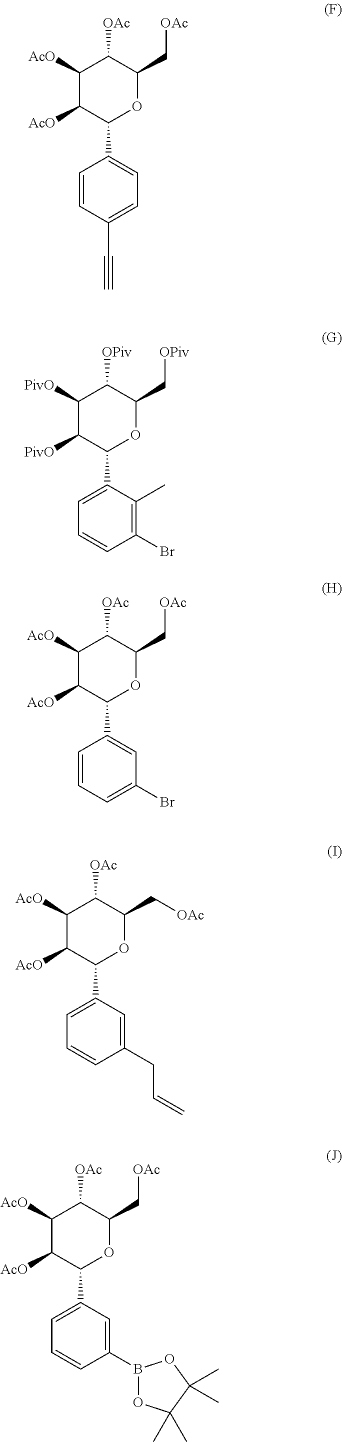

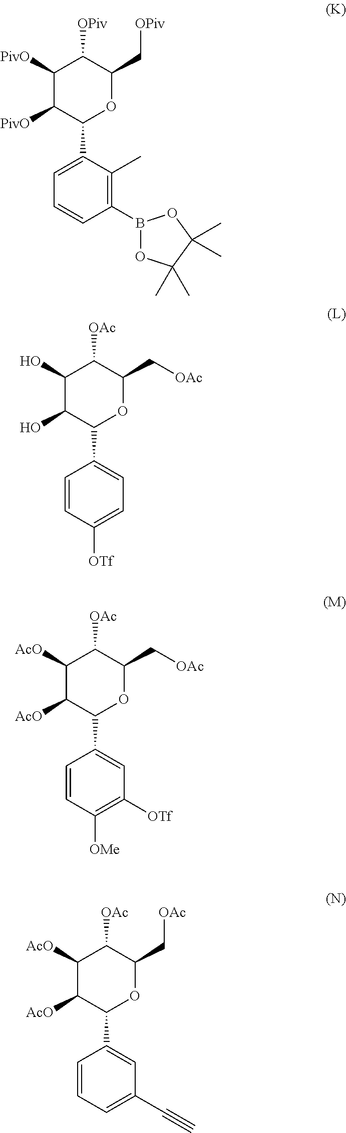

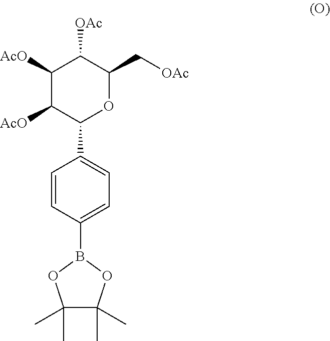 [SMILES: CC(=O)OC[C@H]1O[C@H](c2ccc(B3OC(C)(C)C(C)(C)O3)cc2)[C@@H](OC(C)=O)[C@@H](OC(C)=O)[C@@H]1OC(C)=O]

### Preparation of Intermediate A

### ((2R,3S,4R,5S,6R)-3-acetoxy-4,5-dihydroxy-6-(3-hydroxyphenyl)tetrahydro-2H-pyran-2-yl)methyl acetate

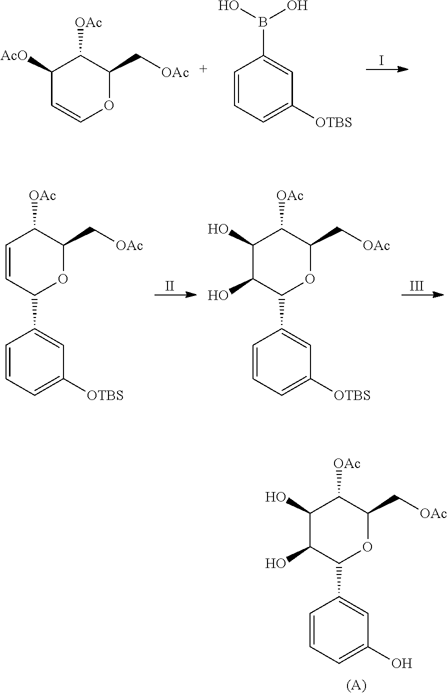

### Step I: [(2R,3S,6S)-3-acetoxy-6-[3-[tert-butyl(dimethyl)silyl]oxyphenyl]-3,6-dihydro-2H-pyran-2-yl]methyl acetate

To a solution of [(2R,3S,4R)-3,4-diacetoxy-3,4-dihydro-2H-pyran-2-yl]methyl acetate (1.100 g, 4.040 mmol) in 10 mL of acetonitrile are added [3-(tert-butyl-dimethyl-silyl)oxyphenyl]boronic acid (2.038 g, 8.080 mmol) and Pd(OAc)₂ (136.1 mg, 0.6060 mmol). The mixture is stirred at rt for 5 h and then to it are added another batch of Pd(OAc)₂ (136 mg, 0.606 mmol) and [3-(tert-butyl-dimethyl-silyl)oxyphenyl]boronic acid (2.038 g, 8.080 mmol). It is then stirred at rt overnight. The mixture is diluted with 20 mL of CH₂Cl₂ and filtered over a pad of celite. The filtrate is concentrated and the residue is separated on Biotage™ SNAP 100 g silica gel cartridge using a gradient of Hex/EtOAc (0-20%) in 20 column volume to afford the title compound (805 mg, 1.91 mmol, 47%) as an oil, which solidifies upon standing.

¹H NMR (CDCl₃, 400 MHz): 7.06 (m, 1H), 6.78 (m, 1H), 6.70 (m, 1H), 6.60 (m, 1H), 5.97 (m, 1H), 5.71 (m, 1H), 5.09 (m, 2H), 4.08 (m, 1H), 3.85 (m, 1H), 3.62 (m, 1H), 1.88 and 1.87 (2s, 6H), 0.78 (m, 9H), 0.00 (m, 6H).

### Step II: [(2R,3S,4R,5S,6R)-3-acetoxy-6-[3-[tert-butyl(dimethyl)silyl]oxyphenyl]-4,5-dihydroxy-tetrahydropyran-2-yl]methyl acetate

To a solution of [(2R,3S,6S)-3-acetoxy-6-[3-[tert-butyl(dimethyl)silyl]oxyphenyl]-3,6-dihydro-2H-pyran-2-yl]methyl acetate (2.500 g, 5.944 mmol) in water (10 mL)/t-BuOH (10 mL) are added methanesulfonamide (848.0 mg, 8.92 mmol), 2.5% OsO₄/t-BuOH (1.87 mL, 0.149 mmol), NMO (1.393 g, 11.89 mmol) and lutidine (689 μL, 5.94 mmol). The mixture is stirred at rt overnight. It is then quenched with 15% sodium bisulfite (15 mL) and diluted with EtOAc (40 mL). The aqueous phase is then separated, washed with water (20 mL) and brine (20 mL) consecutively, dried over Na₂SO₄. After removal of the solvent under reduced pressure, the residue is purified on Biotage™ SNAP 100 g silica gel cartridge using a gradient of CH₂Cl₂/MeOH (0-6%) in 20 column volume to afford the title compound (2.200 g, 81%) as an oil.

¹H NMR (CD₃OD, 400 MHz): 7.06 (m, 1H), 6.78 (m, 1H), 6.70 (m, 1H), 6.58 (m, 1H), 4.85 (m, 1H), 4.64 (m, 1H), 4.46 (m, 1H), 3.96 (m, 1H), 3.85 (m, 1H), 3.62 (m, 2H), 1.86 and 1.83 (2s, 6H), 0.78 (m, 9H), 0.00 (m, 6H).

### Step III: Intermediate A

To a solution of [(2R,3S,4R,5S,6R)-3-acetoxy-6-[3-[tert-butyl(dimethyl)silyl]oxyphenyl]-4,5-dihydroxy-tetrahydropyran-2-yl]methyl acetate (85 mg, 0.19 mmol) in 2 mL of THF are added acetic acid (11 uL, 0.19 mmol) and 1M TBAF/THF (380 μL, 0.38 mmol). After stirring for 1 h, the solvent is removed under reduced pressure and the residue is purified on Biotage™ SNAP 25 g silica gel cartridge using a gradient of CH2CL2/MeOH (0-7%) in 20 column volume to afford INTERMEDIATE A (50 mg, 77%) as an oil.

¹H NMR (CD₃OD, 400 MHz): 7.18 (m, 1H), 6.86 (m, 2H), 6.70 (m, 1H), 5.07 (m, 1H), 4.86 (m, 1H), 4.60 (m, 1H), 4.21 (m, 1H), 4.08 (m, 1H), 3.80 (m, 2H), 2.05 and 2.03 (2s, 6H).

### Preparation of Intermediate B

### ((2R,3S,4R,5S,6R)-3-acetoxy-4,5-dihydroxy-6-(3-(trifluoromethylsulfonyloxy)phenyl)tetrahydro-2H-pyran-2-yl)methyl acetate

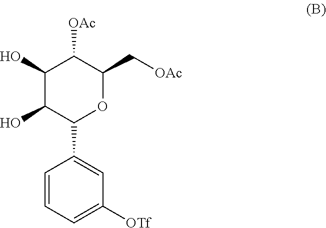

To a solution of INTERMEDIATE A (204 mg, 0.599 mmol) in 5 mL of CH₂Cl₂ are added 1,1,1-trifluoro-N-phenyl-N(trifluoromethylsulfonyl)methanesulfonamide (278 mg, 0.779 mmol) and TEA (167 μL, 1.20 mmol). The mixture is stirred at rt overnight. After removal of the solvent under reduced pressure, the residue is separated on Biotage™ SNAP 25 g silica gel cartridge using a gradient of Hex/EtOAc (0-50%) in 20 column volume to afford INTERMEDIATE B (242 mg, 85%) as a solid.

¹H NMR (CD₃OD, 400 MHz): 7.52 (m, 1H), 7.42 (m, 1H), 7.29 (m, 1H), 5.07 (m, 1H), 4.92 (m, 1H), 4.08 (m, 1H), 3.95 (m, 2H), 3.86 (m, 1H), 2.08 and 2.00 (2s, 6H).

### Preparation of Intermediate C

### (2R,3S,4R,5S,6R)-2-(3-bromophenyl)-6-(hydroxymethyl)tetrahydro-2H-pyran-3,4,5-triol

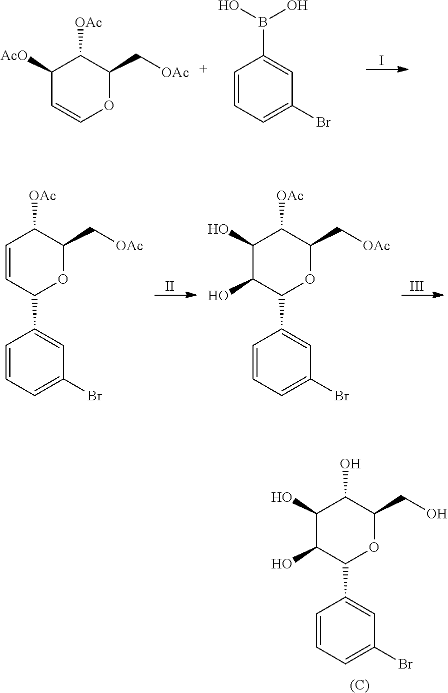

### Step I: ((2R,3S,6S)-3-acetoxy-6-(3-bromophenyl)-3,6-dihydro-2H-pyran-2-yl)methyl acetate

A solution of [(2R,3S,4R)-3,4-diacetoxy-3,4-dihydro-2H-pyran-2-yl]methyl acetate (3.00 g, 11.02 mmol) and (3-bromophenyl)boronic acid (4.426 g, 22.04 mmol) in acetonitrile (22 mL) is degassed by bubbling nitrogen gas through for 3 mins. Palladium (II) acetate (371 mg, 1.65 mmol) is added and the reaction mixture is stirred at rt for 5 h then another portion of palladium (II) acetate (371 mg, 1.65 mmol) is added and stirring is continued for 18 h. The solvent is evaporated and the mixture is diluted with dichloromethane (10 mL) and saturated aqueous NaHCO₃ (20 mL). The mixture is filtered through a phase separator cartridge, the filtrate is evaporated and purified on a Biotage™ Chromatography using 50 g silica gel cartridge using a gradient elution of 5%-10% EtOAc/Hex with a flow rate of 40 mL/min over 30 mins to afford the title product as an oil (1.61 g, 4.36 mmol, 40%).

LC-MS: m/z=391.1, 393.1 (M+Na⁺)

### Step II: ((2R,3S,4R,5S,6R)-3-acetoxy-6-(3-bromophenyl)-4,5-dihydroxytetrahydro-2H-pyran-2-yl)methyl acetate

To a solution of [(2R,3S,65)-3-acetoxy-6-(3-bromophenyl)-3,6-dihydro-2H-pyran-2-yl]methyl acetate (1.60 g, 4.33 mmol) in water (3.1 mL) and THF (19 mL) is added methanesulfonamide (618 mg, 6.50 mmol), osmium tetroxide (1.32 mL of 2.5% w/v in t-BuOH, 0.130 mmol) and N-Methylmorpholine-N-oxide (2.030 g, 17.33 mmol) and the reaction mixture is stirred at rt for 2 days. Another portion of osmium tetroxide (1.32 mL of 2.5% w/v in t-BuOH, 0.130 mmol), methanesulfonamide (618 mg, 6.50 mmol) and N-Methylmorpholine-N-oxide (2.030 g, 17.33 mmol) are added and the mixture is stirred for a further 24 h. The solvent is evaporated and the crude mixture is diluted with a dilute solution of sodium bisulfate (50 mL) and extracted with EtOAc (3×15 mL). The combined organic extracts are dried over Na₂SO₄ and the solvent is evaporated. The gel-like material obtained is dissolved in a minimum amount of MeOH and diluted with diethyl ether and placed in the fridge for 2 h. The mixture is filtered and washed with diethyl ether and dried under high vacuum to afford the title product as a solid (1.480 g, 85%).

LC-MS: m/z=425.1, 427.1 (M+Na⁺)

### Step III: Intermediate C

((2R,3S,4R,5S,6R)-3-acetoxy-6-(3-bromophenyl)-4,5-dihydroxytetrahydro-2H-pyran-2-yl)methyl acetate (1.48 g) is dissolved in MeOH (20 mL) and MeONa in MeOH (187 μL of 25% w/v, 0.87 mmol) is added and the reaction mixture is stirred at rt for 4 h. The reaction mixture is neutralized by the addition of Amberlite IR120H resin until the pH changed to neutral. The reaction mixture is filtered, the filtrate is evaporated and the solid is triturated with Et₂O (2×10 mL) to afford the title product as a solid (1.08 g, 3.046 mmol, 70.3%).

¹H NMR (400 MHz, CD₃OD) δ 7.68 (s, 1H), 7.45 (dd, J=10.6, 4.1 Hz, 2H), 7.29 (t, J=7.9 Hz, 1H), 4.91 (d, J=4.7 Hz, 1H), 4.29 (dd, J=4.6, 3.2 Hz, 1H), 3.95-3.71 (m, 3H), 3.61 (dd, J=7.4, 3.1 Hz, 1H), 3.55-3.47 (m, 1H). LC-MS: m/z=341.1, 343.1 (M+Na⁺)

### Preparation of Intermediate D

### 3-((2R,3S,4R,5S,6R)-5-acetoxy-6-(acetoxymethyl)-3,4-dihydroxytetrahydro-2H-pyran-2-yl)benzoic acid

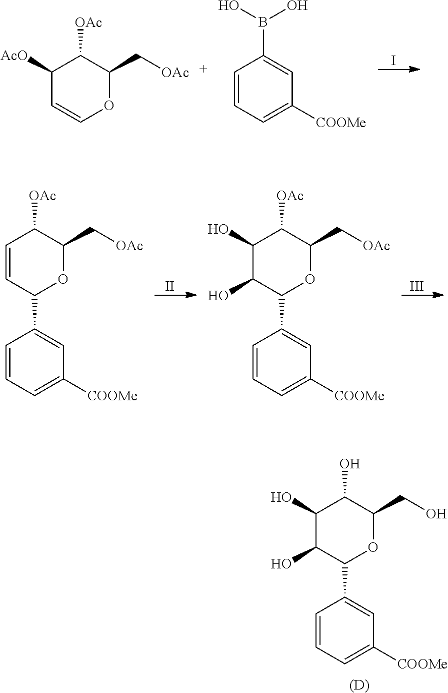

### Step I: Methyl 3-[(2R,3S,6S)-3-acetoxy-2-(acetoxymethyl)-3,6-dihydro-2H-pyran-6-yl]benzoate

The title compound of this step is prepared using the same procedure as described in Step I for Preparation of INTERMEDIATE C, but using (3-methoxycarbonylphenyl)boronic acid as the starting material.

LC-MS: m/z=371.2 (M+Na⁺)

### Step II: Methyl 3-[(2R,3S,4R,5S,6R)-5-acetoxy-6-(acetoxymethyl)-3,4-dihydroxy-tetrahydropyran-2-yl]benzoate

The title compound is prepared using the same procedure as described in Step II for INTERMEDIATE C.

LC-MS: m/z=383.3 (M+H⁺)

### Step III: Intermediate D

A mixture of methyl 3-[(2R,3S,4R,5S,6R)-5-acetoxy-6-(acetoxymethyl)-3,4-dihydroxy-tetrahydropyran-2-yl]benzoate (2.20 g, 5.75 mmol) in MeOH (30 mL) is treated with MeONa in MeOH (341 μL of 25% w/v, 1.58 mmol) and the reaction mixture is stirred at rt for 18 h. The volatiles are evaporated, the mixture is dissolved in MeOH (30 mL), sodium hydroxide (5.13 mL of 2 M, 10.3 mmol) is added and the reaction mixture is stirred at rt for 15 h. The mixture is neutralized by the addition of Amberlite IR120H resin until the pH changed to neutral. The reaction mixture is filtered and the filtrate is evaporated to afford the title product (1.57 g, 5.25 mmol, 66%) as a white solid.

¹H NMR (400 MHz, CD₃OD) δ 8.15 (s, 1H), 7.96 (d, J=7.7 Hz, 1H), 7.75 (d, J=7.7 Hz, 1H), 7.51 (t, J=7.8 Hz, 1H), 4.99 (d, J=4.4 Hz, 1H), 4.42-4.36 (m, 1H), 3.93-3.74 (m, 3H), 3.63 (dd, J=7.5, 3.1 Hz, 1H), 3.54-3.49 (m, 1H). LC-MS: m/z=285.2 (M+H⁺)

### Preparation of Intermediate E

### ((2R,3S,4R,5S,6R)-3-acetoxy-4,5-dihydroxy-6-(2-(trifluoromethylsulfonyloxy)phenyl)tetrahydro-2H-pyran-2-yl)methyl acetate

The title compound is prepared according to the procedures described for INTERMEDIATE A and B but using [2-(tert-butyl-dimethyl-silyl)oxyphenyl]boronic acid in Step I of the synthetic sequence described for INTERMEDIATE A.

### Preparation of Intermediate F

### ((2R,3R,4R,5R,6R)-2-(Acetoxymethyl)-6-(4-ethynylphenyl)tetrahydro-2H-pyran-3,4,5-triyl triacetate)

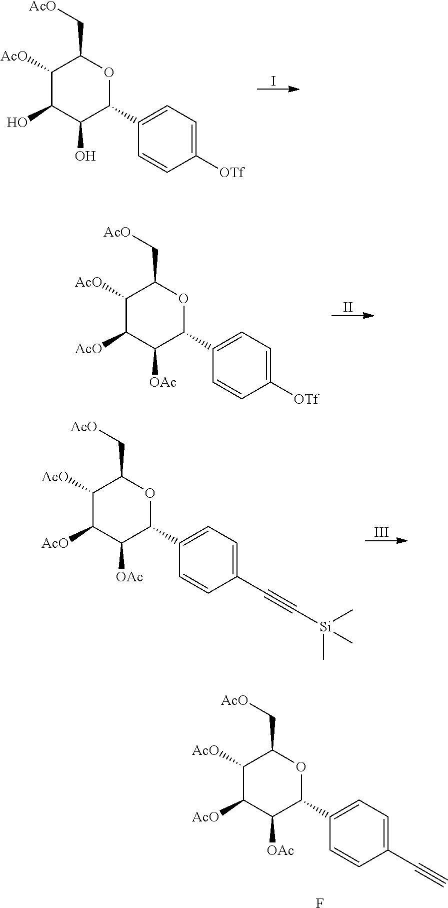

### Step I: [(2R,3R,4R,5R,6R)-3,4,5-triacetoxy-6-[4-(trifluoromethylsulfonyloxy)phenyl]tetrahydropyran-2-yl]methyl acetate

To a solution of (2R,3S,4R,5S,6R)-3-acetoxy-4,5-dihydroxy-6-[4-(trifluoromethylsulfonyloxy)phenyl]tetrahydropyran-2-yl]methyl acetate (256 mg, 0.542 mmol) in 2.6 mL of CH₂Cl₂ is sequentially added pyridine (132 μL, 1.63 mmol), Ac₂O (128 μL, 1.36 mmol) and DMAP (6.6 mg, 0.054 mmol). The reaction mixture is stirred at room temperature for 2 h, diluted with water (1 mL) and the organic layer is dried over Na₂SO₄, filtered, and concentrated to dryness. The residue is purified by flash column chromatography on silica gel (10 to 80% EtOAc in hexanes) to afford the tile compound. (232 mg, 77%).

### Step II: (2R,3R,4R,5R,6R)-2-(acetoxymethyl)-6-(4-((trimethylsilyl)ethynyl)phenyl)tetrahydro-2H-pyran-3,4,5-triyltriacetate

To a mixture of [(2R,3R,4R,5R,6R)-3,4,5-triacetoxy-6-[4-(trifluoromethylsulfonyloxy)phenyl]tetrahydropyran-2-yl]methyl acetate (1217 mg, 2.187 mmol), Pd(dppf)Cl₂—CH₂Cl₂ (178.6 mg, 0.219 mmol) and CuI (83.3 mg, 0.437 mmol) in 12 mL of DMF is added Et₃N (1.5 mL, 11 mmol) followed by ethynyl(trimethyl)silane (1.54 mL, 10.9 mmol). The reaction mixture is heated at 70° C. in a sealed tube for 21 h, cooled to RT, and diluted with water (40 mL). The reaction mixture is extracted by EtOAc (5×20 mL), and the combined organic layer are washed with water (3×10 mL), brine, dried over Na₂SO₄, filtered, and concentrated to dryness. The residue is purified by flash column chromatography on silica gel using a gradient of ethyl acetate in hexanes (10 to 80%) to afford the tile compound (1.0596 g, 96%).

### Step III. Intermediate F

To a solution of (2R,3R,4R,5R,6R)-2-(acetoxymethyl)-6-(4-((trimethylsilyl)ethynyl)phenyl)tetrahydro-2H-pyran-3,4,5-triyltriacetate (1.054 g, 2.089 mmol) in THF (21 mL) is sequentially added AcOH (150.6 mg, 143 μL, 2.507 mmol) and TBAF 1M in THF (2.298 mL of 1 M, 2.298 mmol) under nitrogen atmosphere. The reaction mixture is stirred at room temperature for 2 h, and concentrated to dryness. The residue is purified by flash column chromatography on silica gel using ethyl acetate in hexanes (10 to 80%) to give (2R,3R,4R,5R,6R)-2-(acetoxymethyl)-6-(4-ethynylphenyl)tetrahydro-2H-pyran-3,4,5-triyl triacetate (892 mg, 99%).

### Preparation of Intermediate G

### [(2R,3R,4R,5R,6R)-6-(3-Bromo-2-methyl-phenyl)-3,4,5-tris(2,2-dimethylpropanoyloxy)tetrahydropyran-2-yl]methyl 2,2-dimethylpropanoate

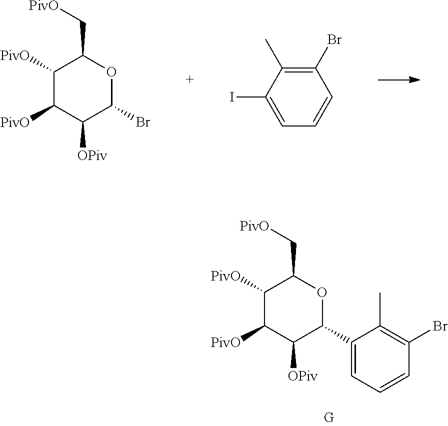

A solution of n-Bu₃MgLi (6.6 mL of 0.66 M, 4.35 mmol) in hexane-heptane-dibutylether (8:20:3) is added to 1-bromo-3-iodo-2-methyl-benzene (3.69 g, 12.42 mmol) in toluene (6.0 mL) and dibutylether (3.6 mL) at 0° C. and stirred at the same temperature for 3.5 h. A solution of ZnBr₂—LiBr in dibutyl ether (6.51 mL of 1.05 M, 6.83 mmol) is added dropwise, cooling bath removed and stirred at RT for 1 h. A solution of [(2R,3R,4S,5S,6R)-6-bromo-3,4,5-tris(2,2-dimethylpropanoyloxy)tetrahydropyran-2-yl]methyl 2,2-dimethylpropanoate (6 g, 10.35 mmol, Ref. Sebastien Lemaire et. al. Org. Letts. 2012, 14, 1480-1483) in toluene (10.8 mL) is added, it is placed on pre-heated oil bath at 90° C. and stirred for 24 h. The reaction mixture is cooled to RT and poured into aq. 1N HCl solution (80 mL), extracted with ethyl acetate (3×50 mL), combined extracts are washed with brine, dried (Na₂SO₄) and concentrated. The residue is purified on Biotage SNAP™ 300 g silica gel cartridge using isocratic ethyl acetate in hexanes (10%, 4 CV) as eluent to afford title compound (4.5 g, 64.9%) as light yellow solid.

1H NMR (400 MHz, CDCl₃) δ 7.55 (d, J=8.0 Hz, 1H), 7.49 (d, J=7.7 Hz, 1H), 7.11 (t, J=7.9 Hz, 1H), 5.71-5.65 (m, 1H), 5.43 (dd, J=6.9, 2.8 Hz, 1H), 5.30-5.23 (m, 1H), 5.20 (d, J=6.0 Hz, 1H), 4.68-4.58 (m, 1H), 4.07 (dd, J=12.0, 3.1 Hz, 1H), 3.87-3.79 (m, 1H), 2.53 (s, 3H), 1.25 (s, 9H), 1.22 (s, 9H), 1.18 (s, 9H), 1.10 (s, 9H).

### Preparation of Intermediate H

### ([(2R,3R,4R,5R,6R)-3,4,5-Triacetoxy-6-(3-bromophenyl)tetrahydropyran-2-yl]methyl acetate)

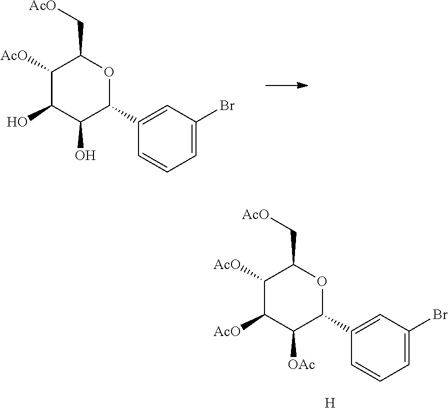

To a solution of [(2R,3S,4R,5S,6R)-3-acetoxy-6-(3-bromophenyl)-4,5-dihydroxy-tetrahydropyran-2-yl]methyl acetate (INTERMEDIATE C, Step 2) (604.8 mg, 1.5 mmol) in THF (10 mL) are added DIPEA (969.3 mg, 1.31 mL, 7.50 mmol), DMAP (18.3 mg, 0.150 mmol) and Ac₂O (536.0 mg, 495 μL, 5.25 mmol) at 0° C. The mixture is stirred at ROOM TEMPERATURE overnight. Then it is quenched with saturated sodium bicarbonate solution. The mixture is extracted with CH₂Cl₂ (3×15 mL). The combined organic extracts are washed with water and brine consecutively, dried over sodium sulfate, filtered, and concentrated to dryness. The residue is separated on Biotage SNAP 25 g silica gel cartridge using a gradient of ethyl acetate in hexanes (0-30%, 20 CV) to obtain title compound (650 mg, 88.9%).

¹H NMR (400 MHz, CDCl₃) δ 7.66 (s, 1H), 7.55-7.45 (m, 1H), 7.41 (dd, 1H), 7.29 (t, 1H), 5.87 (t, 1H), 5.28 (t, 1H), 5.10 (dd, 1H), 5.04 (d, 1H), 4.36 (dd, 1H), 4.14 (dd, 1H), 3.86-3.66 (m, 1H), 2.13 (2s, 6H), 2.05 (s, 3H), 2.02 (s, 3H)

### Preparation of Intermediate I

### [(2R,3R,4R,5R,6R)-3,4,5-Triacetoxy-6-(3-allylphenyl)tetrahydropyran-2-yl]methyl acetate

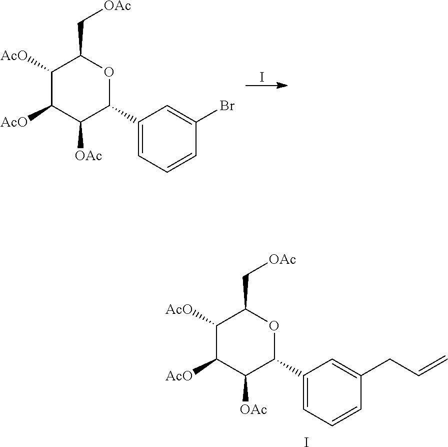

To a degassed (house vacuum/nitrogen) solution of INTERMEDIATE H (320 mg, 0.657 mmol) and allyl-tributyl-stannane (261 mg, 244.0 μL, 0.788 mmol) in benzene (10 mL) is added Pd(PPh₃)₄ (75.9 mg, 0.066 mmol) in one portion, reaction mixture is heated at 100° C. for 48 hours, concentrated. Purified on 50 g silica gel SNAP cartridge on SP1 system using a gradient of ethyl acetate in hexanes (15% to 40%, 8 CV; 40% 4 CV) as eluent to afford title compound (300 mg, quant.) as light yellow oil.

¹H NMR (400 MHz, CDCl₃) δ 7.40-7.31 (m, 3H), 7.21-7.15 (m, 1H), 6.04-5.93 (m, 2H), 5.35 (t, J=9.0 Hz, 1H), 5.16 (dd, J=9.2, 3.2 Hz, 1H), 5.13-5.04 (m, 3H), 4.37 (dt, J=12.1, 7.7 Hz, 1H), 4.14 (dd, J=12.1, 2.7 Hz, 1H), 3.81-3.74 (m, 1H), 3.43 (d, J=6.7 Hz, 2H), 2.17 (s, 3H), 2.13 (s, 3H), 2.06 (s, 3H), 2.02 (s, 3H).

### Preparation of Intermediate J

### [(2R,3R,4R,5R,6R)-3,4,5-triacetoxy-6-[3-(4,4,5,5-tetramethyl-1,3,2-dioxaborolan-2-yl)phenyl]tetrahydropyran-2-yl]methyl acetate

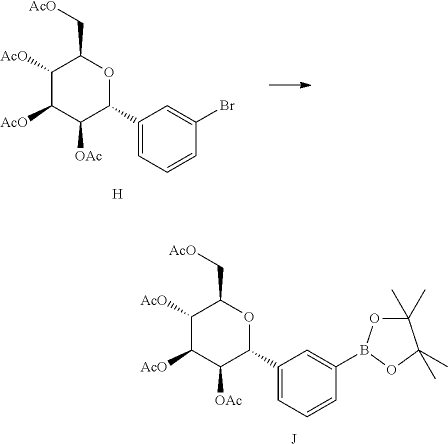

To a solution of INTERMEDIATE H (6.64 g, 13.63 mmol) in DMF (99.6 mL) under nitrogen atmosphere is sequentially added 4,4,5,5-tetramethyl-2-(4,4,5,5-tetramethyl-1,3,2-dioxaborolan-2-yl)-1,3,2-dioxaborolane (5.191 g, 20.44 mmol), KOAc (5.351 g, 54.52 mmol), PdCl₂(dppf) (1.113 g, 1.363 mmol) and the mixture is heated to 60° C. for 22 hours. The mixture is then filtered over Celite, the filtrate is washed with 3 portions of 100 mL of hexanes, diluted with 300 mL of EtOAc, washed with NH₄Cl 20% (100 mL), water (2×100 mL), brine (100 mL), dried over Na₂SO₄ and concentrated to dryness. The residue is purified by flash column chromatography on silica gel (7 to 60% AcOEt in hexanes) to the tile compound (7.283 g).

### Preparation of Intermediate K

### [(2R,3R,4R,5R,6R)-3,4,5-Tris(2,2-dimethylpropanoyloxy)-6-[2-methyl-3-(4,4,5,5-tetramethyl-1,3,2-dioxaborolan-2-yl)phenyl]tetrahydropyran-2-yl]methyl 2,2-dimethylpropanoate

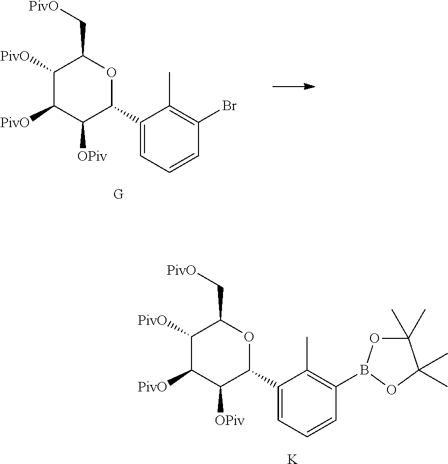

The INTERMEDIATE K (637 mg) is prepared starting from the INTERMEDIATE G as described for the preparation of INTERMEDIATE J.

¹H NMR (400 MHz, CDCl₃) δ 7.72 (d, J=7.3 Hz, 1H), 7.61 (d, J=7.7 Hz, 1H), 7.24 (t, 1H), 5.80-5.73 (m, 1H), 5.46 (dd, J=7.2, 2.9 Hz, 1H), 5.31 (t, J=6.6 Hz, 1H), 5.23 (d, J=5.3 Hz, 1H), 4.51 (dd, J=11.7, 6.8 Hz, 1H), 4.12 (dd, J=12.0, 3.3 Hz, 1H), 3.84-3.74 (m, 1H), 2.62 (s, 3H), 1.34 (s, 12H), 1.24 (s, 9H), 1.21 (s, 9H), 1.19 (s, 9H), 1.12 (s, 9H).

### Preparation of Intermediate L

### ((2R,3S,4R,5S,6R)-3-acetoxy-4,5-dihydroxy-6-(4-(((trifluoromethyl)sulfonyl)oxy)phenyl)tetrahydro-2H-pyran-2-yl)methyl acetate

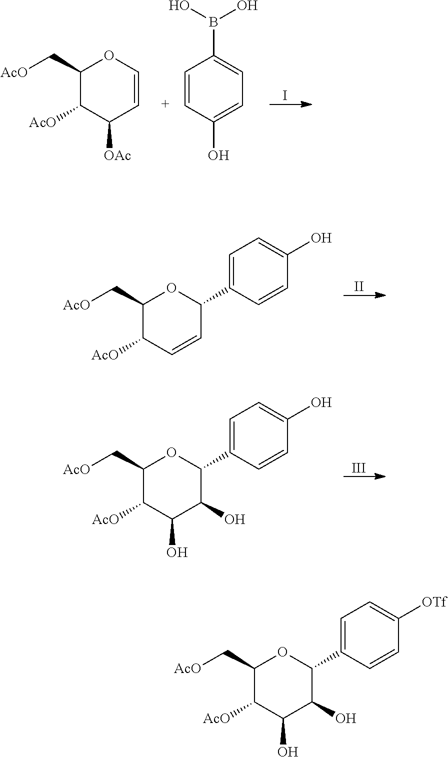

### Step I: [(2R,3S,65)-3-acetoxy-6-(4-hydroxyphenyl)-3,6-dihydro-2H-pyran-2-yl]methyl acetate

Acetonitrile (50.00 mL) is added to a mixture of [(2R,3S,4R)-3,4-diacetoxy-3,4-dihydro-2H-pyran-2-yl]methyl acetate (9.869 g, 36.25 mmol), (4-hydroxyphenyl)boronic acid (5 g, 36.25 mmol) and Pd(OAc)2 (1.221 g, 5.438 mmol) and the reaction mixture is stirred at room temperature overnight. An additional amount of (4-hydroxyphenyl)boronic acid (1 g) is added and the reaction mixture is stirred for a further 2 h and filtered through celite. The filtrate is evaporated and the crude product is purified on a Biotage™ Chromatography system using 340 g silica gel cartridge with a gradient of 5%-80% EtOAc in Hexanes to afford title product.

### Step II: [(2R,3S,4R,5S,6R)-3-Acetoxy-4,5-dihydroxy-6-(4-hydroxyphenyl)tetrahydropyran-2-yl]methyl acetate

To a suspension of [(2R,3S,6S)-3-acetoxy-6-(4-hydroxyphenyl)-3,6-dihydro-2H-pyran-2-yl]methyl acetate (6.03 g, 19.69 mmol) in THF (36 mL)/water (24 mL) are added methanesulfonamide (2.810 g, 29.54 mmol), OsO4 (6.007 g, 7.4 mL of 2.5% w/w in -t-BuOH, 0.5907 mmol) and NMO (4.613 g, 39.38 mmol). The reaction mixture is stirred at room temperature overnight. 1M Na2S2O3 (40 mL) is added and the mixture is extract with EtOAc (3×40 mL). The combined organic extracts are washed with brine (15 mL) and dried over Na2SO4. The mixture is filtered, the solvent is evaporated and the crude product is purified on a Biotage™ Chromatography system using 220 g silica gel cartridge with a gradient of 0%-20% MeOH in CH₂Cl₂ to afford title product.

LC-MS: m/z=329.3 (M+Na+)

### Step III: Intermediate L

To a solution of [(2R,3S,4R,5S,6R)-3-acetoxy-4,5-dihydroxy-6-(4-hydroxyphenyl)tetrahydropyran-2-yl]methyl acetate (872 mg, 2.562 mmol) in CH₂Cl₂ (22 mL) are added 1,1,1-trifluoro-N-phenyl-N-(trifluoromethylsulfonyl)methanesulfonamide (1.190 g, 3.331 mmol), NEt3 (518.5 mg, 714 μL, 5.124 mmol) and the reaction mixture is stirred at room temperature overnight. The solvent is evaporated and the crude product is purified on a Biotage™ Chromatography system using 100 g silica gel cartridge with a gradient of 0%-20% MeOH/CH₂Cl₂ over 15 column volume to afford the title product.

### Preparation of Intermediate M

### [(2R,3R,4R,5R,6R)-3,4,5-triacetoxy-6-[4-methoxy-3-(trifluoromethylsulfonyloxy)phenyl]tetrahydropyran-2-yl]methyl acetate

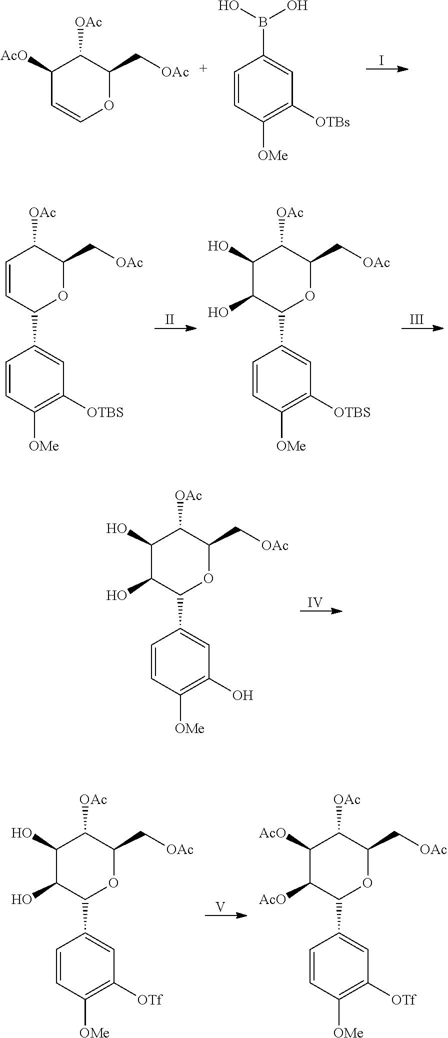

### Step I: [(2R,3S,6S)-3-acetoxy-6-[3-[tert-butyl(dimethyl)silyl]oxy-4-methoxy-phenyl]-3,6-dihydro-2H-pyran-2-yl]methyl acetate

To a solution of [(2R,3S,4R)-3,4-diacetoxy-3,4-dihydro-2H-pyran-2-yl]methyl acetate (2 g, 7.346 mmol) in 35 mL of ACN are added [3-[tert-butyl(dimethyl)silyl]oxy-4-methoxy-phenyl]boronic acid (2.073 g, 7.346 mmol) and Pd(OAc)2 (247.4 mg, 1.102 mmol). The mixture is stirred at room temperature overnight and then to it are added another batch of Pd(OAc)2 (247.4 mg, 1.102 mmol) and [3-[tert-butyl(dimethyl)silyl]oxy-4-methoxy-phenyl]boronic acid (2.073 g, 7.346 mmol). It is then stirred at room temperature overnight again. The mixture is diluted with 30 mL of CH₂Cl₂ and filtered over a pad of celite. The filtrate is concentrated and the residue is separated on Biotage plug 100 g (a silica gel cartridge) using a gradient of ethyl acetate/hexane at 0-15% in 20 column volume to obtain the title compound.

### Step II: ((2R,3S,4R,5S,6R)-3-acetoxy-6-(3-((tert-butyldimethylsilyl)oxy)-4-methoxyphenyl)-4,5-dihydroxytetrahydro-2H-pyran-2-yl)methyl acetate

To a solution of [(2R,3S,6S)-3-acetoxy-6-[3-[tert-butyl(dimethyl)silyl]oxy-4-methoxy-phenyl]-3,6-dihydro-2H-pyran-2-yl]methyl acetate (1.85 g, 4.106 mmol) in water (7.400 mL)/t-BuOH (7.400 mL) were added methanesulfonamide (585.9 mg, 6.159 mmol), 2.5% OsO4/t-BuOH (1.044 g, 1.289 mL, 0.1027 mmol), NMO (962.0 mg, 8.212 mmol) and lutidine (440.0 mg, 475.7 μL, 4.106 mmol). The mixture was stirred at room temperature for 24 h. It was then quenched with 15% sodium bisulfate (15 mL) and diluted with ethyl acetate. The aqueous phase was separated, washed with water and brine, dried over sodium sulfate. After removal of the solvent under reduced pressure, the residue is purified on Biotage SNAP (silica gel cartridge) 50 g using a gradient of MeOH/CH₂Cl₂ 0-8% in 20 column volume to obtain the title compound.

### Step III: [(2R,3S,4R,5S,6R)-3-acetoxy-4,5-dihydroxy-6-(3-hydroxy-4-methoxy-phenyl)tetrahydropyran-2-yl]methyl acetate

To a solution of [(2R,3S,4R,5S,6R)-3-acetoxy-6-[3-[tert-butyl(dimethyl)silyl]oxy-4-methoxy-phenyl]-4,5-dihydroxy-tetrahydropyran-2-yl]methyl acetate (400 mg, 0.8254 mmol) in CH₂Cl₂ (16.00 mL) was added 1M TBAF/THF (1.651 mL of 1 M, 1.651 mmol) and AcOH (49.57 mg, 46.94 μL, 0.8254 mmol). The reaction mixture was stirred at room temperature for 2 h, washed with H2O, brine, dried over Na2SO4, filtered and dried. The residue was purified on biotage SP (25 g cartridge) using MeOH in CH₂Cl₂ 0 to 5% (20 cv) as eluent to afford the title compound.

### Step IV: [(2R,3S,4R,5S,6R)-3-acetoxy-4,5-dihydroxy-6-[4-methoxy-3-(trifluoromethylsulfonyloxy)phenyl]tetrahydropyran-2-yl]methyl acetate

To a solution of [(2R,3S,4R,5S,6R)-3-acetoxy-4,5-dihydroxy-6-(3-hydroxy-4-methoxy-phenyl)tetrahydropyran-2-yl]methyl acetate (230 mg, 0.6210 mmol) in CH₂Cl₂ (5.750 mL) were added N-[dioxo-(trifluoro-$1̂{4}-sulfanyl)methyl]-1,1,1-trifluoro-N-phenyl-methanesulfonamide (288.4 mg, 0.8073 mmol) and TEA (125.7 mg, 173.1 μL, 1.242 mmol), not completely soluble, addition of CH₂Cl₂ (3.450 mL). The mixture was stirred at room temperature for 2 days. After removal of the solvent under reduced pressure, the residue was purified on Biotage SNAP (25 g cartridge) using a gradient of CH₂Cl₂/MeOH 0-5% in 20 cv (column volume) to obtain the title compound.

### Step V: Intermediate M

To a stirred solution of [(2R,3S,4R,5S,6R)-3-acetoxy-4,5-dihydroxy-6-[4-methoxy-3-(trifluoromethylsulfonyloxy)phenyl]tetrahydropyran-2-yl]methyl acetate (200 mg, 0.3981 mmol) in CH₂Cl₂ (3 mL) at was added sequentially pyridine (100.8 mg, 103.1 μL, 1.274 mmol), acetic anhydride (121.9 mg, 112.7 μL, 1.194 mmol) and DMAP (4.863 mg, 0.03981 mmol), stirred for 16 hours, diluted with water, organic solution was separated with phase separator, aqueous solution was washed with methylene chloride, combined organic solution was concentrated, purified on 25 g SNAP silica gel cartridge using ethyl acetate in hexanes (20% to 50%, 10 CV; 50% 5 CV) as eluent to afford the title compound.

### Preparation of Intermediate N

### ((2R,3S,4R,5S,6R)-2-(3-Ethynylphenyl)-6-(hydroxymethyl)tetrahydropyran-3,4,5-triol)

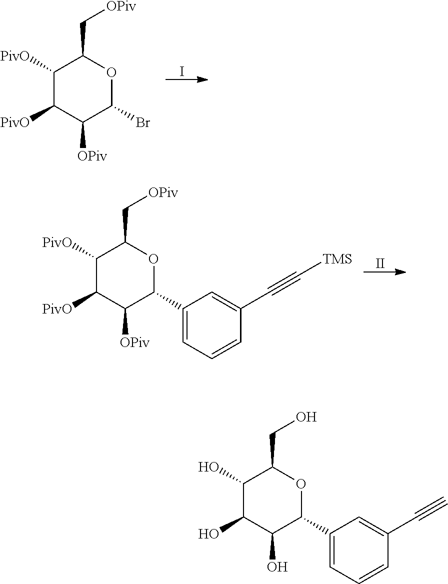

### Step I: (2R,3R,4R,5R,6R)-2-((pivaloyloxy)methyl)-6-(3-((trimethylsilyl)ethynyl)phenyl)tetrahydro-2H-pyran-3,4,5-triyltris(2,2-dimethylpropanoate)

A solution of n-Bu₃MgLi (2.65 mL of 0.65 M, 1.725 mmol) in hexane-heptane-dibutylether (8:20:3) is added to 2-(3-bromophenyl)ethynyl-trimethyl-silane (1.248 g, 1.05 mL, 4.928 mmol) in toluene (2.4 mL) and dibutylether (1.4 mL) at 0° C. and stirred in cold room for 25 h. A solution of ZnBr₂—LiBr in dibutyl ether (2.6 mL of 1.05 M, 2.711 mmol) is added dropwise, cooling bath removed, stirred at room temperature for 1 h. A solution of [(2R,3R,4S,5S,6R)-6-bromo-3,4,5-tris(2,2-dimethylpropanoyloxy)tetrahydropyran-2-yl]methyl 2,2-dimethylpropanoate (2.38 g, 4.107 mmol) in toluene (4.3 mL) is added, it is placed on pre-heated oil bath at 90° C., stirred over weekend. The reaction mixture is cooled to room temperature, it is poured into aq. 1 N HCl solution (40 mL) and extracted with ethyl acetate (3×40 mL). The combined extracts are washed with brine, dried (Na₂SO₄), concentrated, purified on Biotage™ 100 g SNAP silica gel cartridge using ethyl acetate in hexanes (0% to 10%, 12 CV, 10%, 5 CV) as eluent to afford the title compound.

### Step II: (2R,3S,4R,5S,6R)-2-(3-ethynylphenyl)-6-(hydroxymethyl)tetrahydropyran-3,4,5-triol

To a stirred light suspension of [(2R,3R,4R,5R,6R)-3,4,5-tris(2,2-dimethylpropanoyloxy)-6-[3-(2-trimethylsilylethynyl)phenyl]tetrahydropyran-2-yl]methyl 2,2-dimethylpropanoate (765 mg, 1.137 mmol) in methanol (15 mL) is added methanolate (Sodium Ion (1)) (4.6 mL of 0.5 M, 2.274 mmol) and stirred at room temperature for 24 h. To the resultant solution is added DOWEX 50WX4-400 until pH 4-5, filtered, eluted with methanol. The filtrate is concentrated, purified on Biotage™ 40 g silica gel SNAP cartridge using EtOAc-MeOH—H₂O (47.5:1.5:1 to 10:1.5:1) as eluent to afford title compound. LC-MS: m/z=265.28 (M+H⁺).

### Preparation of Intermediate O

### [(2R,3R,4R,5R,6R)-3,4,5-triacetoxy-6-[4-(trifluoromethylsulfonyloxy)phenyl]tetrahydropyran-2-yl]methyl acetate

INTERMEDIATE F (16.54 g, 29.7 mmol), bis(pinacolato)diboron (11.36 g, 44.7 mmol) and KOAc (11.77 g, 119.9 mmol) are combined in DMF (250 mL). The resulting mixture is degassed (vacuum then N2, 3×), then Pd(DPPF)(Cl)₂.CH₂Cl₂ (2.48 g, 3.04 mmol) is added, the mixture is degassed again and stirred at 60° C. for 3.5 h. The reaction mixture is cooled down to room temperature, filtered through a celite plug, rinsing with portions of DMF (total 50 mL). The resulting DMF solution is washed with hexanes (3×250 mL). The DMF layer is diluted with EtOAc (750 mL), washed with saturated aqueous NH₄Cl solution (250 mL), H₂O (2×250 mL), and brine (250 mL), dried over Na₂SO₄, filtered and concentrated to provide crude product which is purified by flash chromatography on a silica Biotage™ snap 340 g cartridge, using a gradient of EtOAc in hexanes (30-40%). Mixed fractions are concentrated and purified on a silica Biotage™ snap 340 g cartridge, using a gradient of EtOAc in CH₂Cl₂ (0-30%). Fractions from the two columns are combined and concentrated, affording the title compound (12.94 g, 81% yield) as a white foamy solid.

### Example 1

### Preparation of Compound 1

### (2R,3S,4R,5S,6R)-2-(hydroxymethyl)-6-(3-hydroxyphenyl)tetrahydropyran-3,4,5-triol

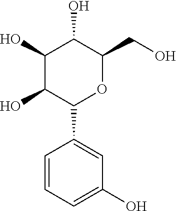

To a solution of INTERMEDIATE A (45 mg, 0.132 mmol) in 3 mL of MeOH is added sodium methoxide (2.9 μL, 0.013 mmol). The mixture is stirred at room temperature for 30 minutes and then it is neutralized with resin Amberlite IR120(H). After filtration, the filtrate is concentrated to dryness under reduced pressure. The residue is purified by reverse phase HPLC to afford the title compound.

¹H NMR (CD₃OD, 400 MHz): δ 7.18 (m, 1H), 6.90 (m, 2H), 6.66 (m, 1H), 4.91 (m, 1H), 4.40 (m, 1H), 3.80 (m, 3H), 3.55 (m, 1H), 3.44 (m, 1H). LC-MS: m/z=257.3 (M+H⁺).

### Example 2

### Preparation of Compound 2

### N-methyl-4-[3-[(2R,3S,4R,5S,6R)-3,4,5-trihydroxy-6-(hydroxymethyl)tetrahydropyran-2-yl]phenyl]benzamide

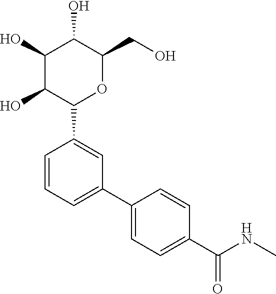

### Step I: ((2R,3S,4R,5S,6R)-3-acetoxy-4,5-dihydroxy-6-(4′-(methylcarbamoyl)biphenyl-3-yl)tetrahydro-2H-pyran-2-yl)methyl acetate

To a solution of INTERMEDIATE B (30 mg, 0.0635 mmol) in 3 mL of dioxane are added [4-(methylcarbamoyl)phenyl]boronic acid (17.1 mg, 0.0953 mmol), 1 M sodium bicarbonate (254 μL, 0.254 mmol) and Pd(PPh₃)₄ (7 mg, 0.0064 mmol). The mixture is stirred at 90° C. overnight under nitrogen. After removal of the solvent under reduced pressure, the residue is purified on Biotage™ SNAP 10 g silica gel cartridge using a gradient of CH₂Cl₂/MeOH (0-8%) in 25 column volume to obtain a mixture (20 mg) of 3 compounds. The mixture is used directly in the next step without further purification.

### Step II: Compound 2

To a solution of the above-mentioned mixture (20 mg) in methanol (3 mL) is added a drop of 25% Sodium methoxide/methanol. After stirring for 20 min, it is neutralized with Amberlite IR120(H). After filtration, the solvent is removed under reduced pressure and the residue is purified by reverse phase HPLC to afford the title compound (11 mg).

¹H NMR (CD₃OD, 400 MHz): δ 7.80 (m, 2H), 7.74 (s, 1H), 7.67 (m, 2H), 7.51 (m, 1H), 7.40 (m, 2H), 4.94 (d, 1H), 4.38 (m, 1H), 3.77 (m, 2H), 3.66 (m, 1H), 3.55 (m, 1H), 3.47 (m, 1H), 2.84 (s, 3H). LC-MS: m/z=374.2 (M+H⁺).

### Example 3

### Preparation of Compound 3

### N-methyl-4-[3-[(2R,3S,4R,5S,6R)-3,4,5-trihydroxy-6-(hydroxymethyl)tetrahydropyran-2-yl]phenoxy]benzamide

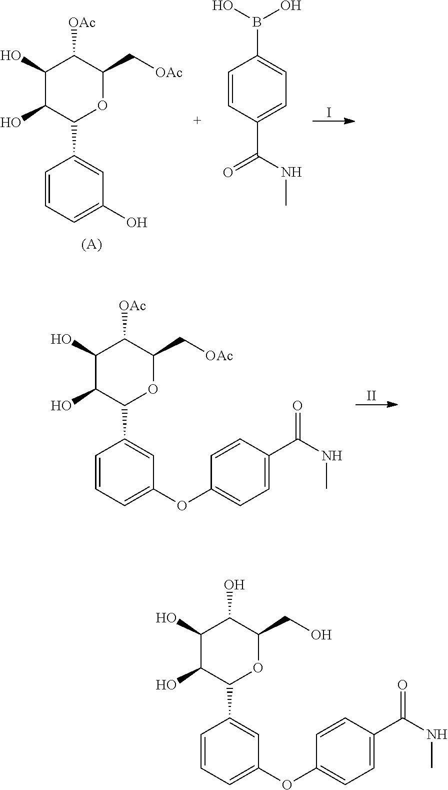

### Step I: ((2R,3S,4R,5S,6R)-3-acetoxy-4,5-dihydroxy-6-(3-(4-(methylcarbamoyl)phenoxy)phenyl)tetrahydro-2H-pyran-2-yl)methyl acetate

To a solution of INTERMEDIATE A (50 mg, 0.147 mmol) in 5 mL of CH₂Cl₂ are added 4-(methylcarbamoyl)phenylboronic acid (53 mg, 0.296 mmol), molecular sieves (300 mg) and Cu(OAc)₂ (37 mg, 0.206 mmol). After stirring for 10 min, lutidine (85 μL, 0.735 mmol) is added to the mixture. The reaction mixture is then stirred at room temperature for 2 days. After removal of the solvent under reduced pressure, the residue is separated on Biotage™ SNAP 25 g silica gel cartridge using a gradient of CH₂Cl₂/MeOH (0-8%) in 20 column volume to obtain a mixture, which contains the desired material based on LC-MS and is used directly in the next step without further purification.

LC-MS: m/z=474.3 (M+H⁺).

### Step II: Compound 3

To a solution of the above-mentioned mixture (50 mg) in methanol (3 mL) is added a drop of 25% MeONa/MeOH. After stirring for 20 min, it is neutralized with Amberlite IR120(H). After filtration, the solvent is removed under reduced pressure and the residue is purified by reverse phase HPLC to afford the title compound (14 mg).

¹H NMR (CD₃OD, 400 MHz): δ 7.79 (m, 2H), 7.42 (m, 1H), 7.30 (m, 1H), 7.20 (m, 1H), 6.98 (m, 3H), 4.94 (d, 1H), 4.32 (m, 1H), 3.74 (m, 3H), 3.60 (m, 1H), 3.47 (m, 1H), 2.84 (s, 3H). LC-MS: m/z=390.3 (M+H⁺).

### Example 4

### Preparation of Compound 4

### (2R,3S,4R,5S,6R)-2-(hydroxymethyl)-6-[3-(3-methylbenzimidazol-5-yl)phenyl]tetrahydropyran-3,4,5-triol (Trifluoroacetic Acid Salt)

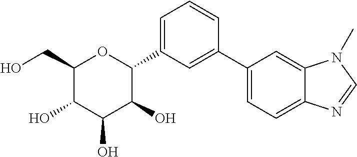 [SMILES: Cn1cnc2ccc(-c3cccc([C@H]4O[C@H](CO)[C@@H](O)[C@H](O)[C@@H]4O)c3)cc21]

The title compound is prepared using similar procedure as described for COMPOUND 2 but using 1-methyl-1H-benzo[d]imidazol-6-ylboronic acid as the appropriate starting material.

¹H NMR (CD₃OD, 400 MHz): δ 9.21 (s, 1H), 8.07 (s, 1H), 7.86 (m, 2H), 7.78 (m, 1H), 7.66 (m, 1H), 7.44 (m, 2H), 4.94 (d, 1H), 4.38 (m, 1H), 4.07 (s, 3H), 3.77 (m, 2H), 3.66 (m, 1H), 3.55 (m, 1H), 3.51 (m, 1H). LC-MS: m/z=371.3 (M+H⁺).

### Example 5

### Preparation of Compound 5

### N-methyl-3-[3-[(2R,3S,4R,5S,6R)-3,4,5-trihydroxy-6-(hydroxymethyl)tetrahydropyran-2-yl]phenoxy]benzamide

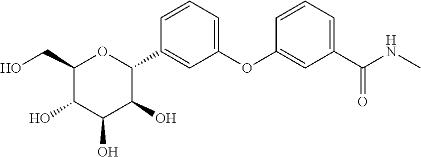 [SMILES: CNC(=O)c1cccc(Oc2cccc([C@H]3O[C@H](CO)[C@@H](O)[C@H](O)[C@@H]3O)c2)c1]

The title compound is prepared using similar procedure as described in COMPOUND 3 but using 3-(methylcarbamoyl)phenylboronic acid.

¹H NMR (CD₃OD, 400 MHz): 7.53 (m, 1H), 7.40 (m, 3H), 7.24 (m, 1H), 7.15 (m, 2H), 7.92 (m, 1H), 4.94 (d, 1H), 4.38 (m, 1H), 3.77 (m, 3H), 3.59 (m, 1H), 3.47 (m, 1H), 2.81 (s, 3H). LC-MS: m/z=390.3 (M+H⁺).

### Example 6

### Preparation of Compound 6

### (2R,3S,4R,5S,6R)-2-(hydroxymethyl)-6-(4′-methyl-[1,1′-biphenyl]-3-yl)tetrahydro-2H-pyran-3,4,5-triol

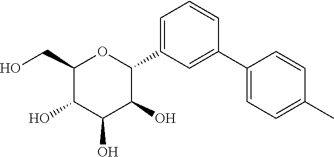 [SMILES: Cc1ccc(-c2cccc([C@H]3O[C@H](CO)[C@@H](O)[C@H](O)[C@@H]3O)c2)cc1]

A mixture of INTERMEDIATE C (126 μL of 0.5 M, 0.063 mmol), p-tolylboronic acid in N-methylpyrrolidine (189 μL of 0.5M, 0.095 mmol), PdCl₂(dppf)₂.CH₂Cl₂ (3.8 mg, 0.0063 mmol) and aqueous Na₂CO₃ (63 μL of 2M, 0.126 mmol) is heated in a 4 mL sealed vial at 90° C. for 15 h. The mixture is filtered through a pad of celite and purified directly by reverse phase HPLC to afford the title compound.

¹H NMR (400 MHz, CD₃OD) δ 7.75 (s, 1H), 7.58-7.49 (m, 3H), 7.44 (dd, J=9.1, 4.4 Hz, 2H), 7.25 (d, J=8.0 Hz, 2H), 5.04 (d, J=3.6 Hz, 1H), 4.50 (t, J=3.4 Hz, 1H), 3.85 (d, J=5.0 Hz, 2H), 3.76 (t, J=7.9 Hz, 1H), 3.64 (dd, J=8.0, 3.1 Hz, 1H), 3.54 (dt, J=7.9, 4.8 Hz, 1H), 2.37 (s, 3H). LC-MS: m/z=353.2 (M+Na⁺)

COMPOUNDS 7 to 22 listed in Table 1 below are prepared using similar procedure described in COMPOUND 6:

TABLE 1

| | | LC-MS: |
|---|---|---|
| COMPOUND | Structure | m/z (M + H⁺) |
| 7 | 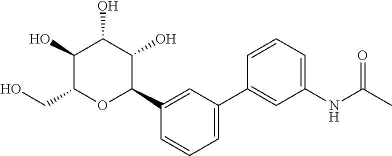 [SMILES: CC(=O)Nc1cccc(-c2cccc([C@H]3O[C@H](CO)[C@@H](O)[C@H](O)[C@@H]3O)c2)c1] N-[3-[3-[(2R,3S,4R,5S,6R)-3,4,5-trihydroxy-6- (hydroxymethyl)tetrahydropyran-2- yl]phenyl]phenyl]acetamide | 374.2 |
| 8 | 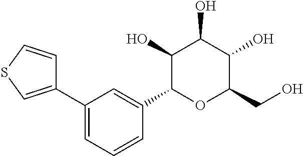 [SMILES: OC[C@H]1O[C@H](c2cccc(-c3ccsc3)c2)[C@@H](O)[C@@H](O)[C@@H]1O] (2R,3S,4R,5S,6R)-2-(hydroxymethyl)-6-[3-(3- thienyl)phenyl]tetrahydropyran-3,4,5-triol | 323.2 |
| 9 | 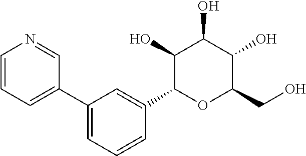 [SMILES: OC[C@H]1O[C@H](c2cccc(-c3cccnc3)c2)[C@@H](O)[C@@H](O)[C@@H]1O] TFA salt (2R,3S,4R,5S,6R)-2-(hydroxymethyl)-6-[3-(3- pyridyl)phenyl]tetrahydropyran-3,4,5-triol | 318.2 |
| 10 | 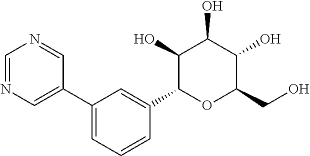 [SMILES: OC[C@H]1O[C@H](c2cccc(-c3cncnc3)c2)[C@@H](O)[C@@H](O)[C@@H]1O] (2R,3S,4R,5S,6R)-2-(hydroxymethyl)-6-(3-pyrimidin-5- ylphenyl)tetrahydropyran-3,4,5-triol | 319.3 |
| 11 | 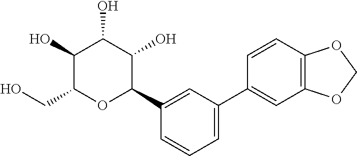 [SMILES: OC[C@H]1O[C@H](c2cccc(-c3ccc4c(c3)OCO4)c2)[C@@H](O)[C@@H](O)[C@@H]1O] (2R,3S,4R,5S,6R)-2-[3-(1,3-benzodioxol-5-yl)phenyl]-6- (hydroxymethyl)tetrahydropyran-3,4,5-triol | 361.2 |
| 12 | 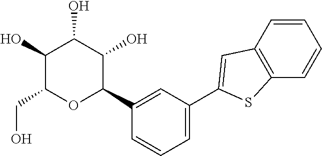 [SMILES: OC[C@H]1O[C@H](c2cccc(-c3cc4ccccc4s3)c2)[C@@H](O)[C@@H](O)[C@@H]1O] (2R,3S,4R,5S,6R)-2-[3-(benzothiophen-2-yl)phenyl]-6- (hydroxymethyl)tetrahydropyran-3,4,5-triol | 373.2 |
| 13 | 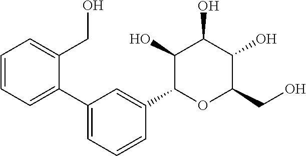 [SMILES: OCc1ccccc1-c1cccc([C@H]2O[C@H](CO)[C@@H](O)[C@H](O)[C@@H]2O)c1] (2R,3S,4R,5S,6R)-2-(hydroxymethyl)-6-(2′- (hydroxymethyl)-[1,1′-biphenyl]-3-yl)tetrahydro-2H-pyran- 3,4,5-triol | 369.2 (M + Na⁺) |
| 14 | 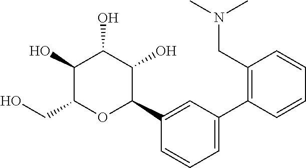 [SMILES: CN(C)Cc1ccccc1-c1cccc([C@H]2O[C@H](CO)[C@@H](O)[C@H](O)[C@@H]2O)c1] TFA salt (2R,3S,4R,5S,6R)-2-(2′-((dimethylamino)methyl)-[1,1′- biphenyl]-3-yl)-6-(hydroxymethyl)tetrahydro-2H-pyran- 3,4,5-triol | 374.3 |
| 15 | 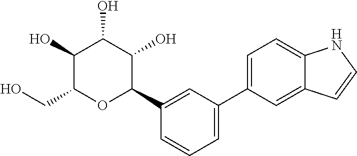 [SMILES: OC[C@H]1O[C@H](c2cccc(-c3ccc4[nH]ccc4c3)c2)[C@@H](O)[C@@H](O)[C@@H]1O] (2R,3S,4R,5S,6R)-2-(3′-amino-[1,1′-biphenyl-3-yl)-6- (hydroxymethyl)tetrahydro-2H-pyran-3,4,5-triol | 356.2 |
| 16 | 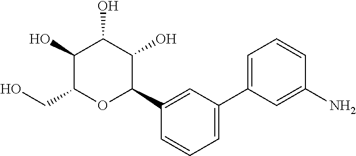 [SMILES: Nc1cccc(-c2cccc([C@H]3O[C@H](CO)[C@@H](O)[C@H](O)[C@@H]3O)c2)c1] (2R,3S,4R,5S,6R)-2-[3-(3-aminophenyl)phenyl]-6- (hydroxymethyl)tetrahydropyran-3,4,5-triol | 332.2 |
| 17 | 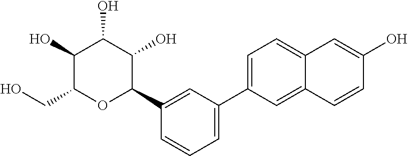 [SMILES: OC[C@H]1O[C@H](c2cccc(-c3ccc4cc(O)ccc4c3)c2)[C@@H](O)[C@@H](O)[C@@H]1O] (2R,3S,4R,5S,6R)-2-(hydroxymethyl)-6-[3-(6-hydroxy-2- naphthyl)phenyl]tetrahydropyran-3,4,5-triol | 383.2 |
| 18 | 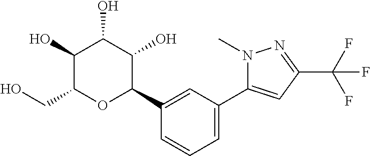 [SMILES: Cn1nc(C(F)(F)F)cc1-c1cccc([C@H]2O[C@H](CO)[C@@H](O)[C@H](O)[C@@H]2O)c1] (2R,3S,4R,5S,6R)-2-(hydroxymethyl)-6-(3-(1-methyl-3- (trifluoromethyl)-1H-pyrazol-5-yl)phenyl)tetrahydro-2H- pyran-3,4,5-triol | 389.2 |
| 19 | 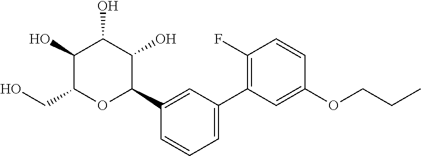 [SMILES: CCCOc1ccc(F)c(-c2cccc([C@H]3O[C@H](CO)[C@@H](O)[C@H](O)[C@@H]3O)c2)c1] (2R,3S,4R,5S,6R)-2-(2′- fluoro-5′-propoxy-[1,1′-biphenyl-3-yl)-6- (hydroxymethyl)tetrahydro-2H-pyran-3,4,5-triol | 415.2 (M + Na⁺) |
| 20 | 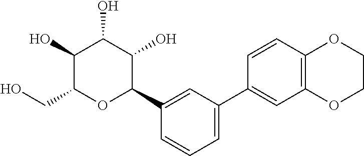 [SMILES: OC[C@H]1O[C@H](c2cccc(-c3ccc4c(c3)OCCO4)c2)[C@@H](O)[C@@H](O)[C@@H]1O] (2R,3S,4R,5S,6R)-2-(3-(2,3-dihydrobenzo[b][1,4]dioxin-6- yl)phenyl)-6-(hydroxymethyl)tetrahydro-2H-pyran-3,4,5- triol | 375.2 |
| 21 | 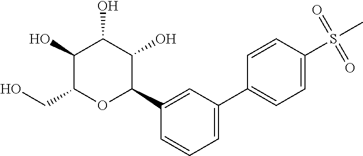 [SMILES: CS(=O)(=O)c1ccc(-c2cccc([C@H]3O[C@H](CO)[C@@H](O)[C@H](O)[C@@H]3O)c2)cc1] (2R,3S,4R,5S,6R)-2-(hydroxymethyl)-6-(4′- (methylsulfonyl)-[1,1′-biphenyl-3-yl)tetrahydro-2H-pyran- 3,4,5-triol | 417.1 (M + Na⁺) |
| 22 | 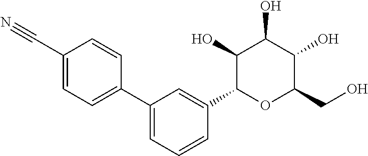 [SMILES: N#Cc1ccc(-c2cccc([C@H]3O[C@H](CO)[C@@H](O)[C@H](O)[C@@H]3O)c2)cc1] 3′-((2R,3S,4R,5S,6R)-3,4,5-trihydroxy-6- (hydroxymethyl)tetrahydro-2H-pyran-2-yl)-[1,1′-biphenyl]- 4-carbonitrile | 364.2 (M + Na⁺) |

### Example 7

### Preparation of Compound 23

### N-phenyl-3-[(2R,3S,4R,5S,6R)-3,4,5-trihydroxy-6-(hydroxymethyl)tetrahydropyran-2-yl]benzamide

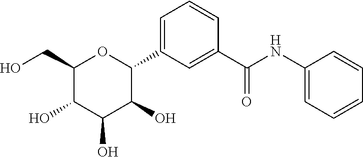 [SMILES: O=C(Nc1ccccc1)c1cccc([C@H]2O[C@H](CO)[C@@H](O)[C@H](O)[C@@H]2O)c1]

To a solution of INTERMEDIATE D in NMP (88 μL of 1M, 0.088 mmol), aniline in NMP (194 μL of 0.5 M, 0.097 mmol), HATU in NMP (114 μL of 1 M, 0.114 mmol) and triethylamine (25 μL, 0.18 mmol) are added. The reaction mixture is stirred at room temperature for 18 hours and purified directly by reverse phase HPLC to afford the title compound (14.4 mg, 41%).

¹H NMR (400 MHz, CD₃OD) δ 8.04 (d, J=14.9 Hz, 1H), 7.86 (d, J=7.8 Hz, 1H), 7.76-7.65 (m, 3H), 7.53 (t, J=7.7 Hz, 1H), 7.36 (t, J=7.9 Hz, 2H), 7.15 (t, J=7.4 Hz, 1H), 5.01 (d, J=7.3 Hz, 1H), 4.42 (dd, J=4.5, 3.2 Hz, 1H), 3.92 (dd, J=11.9, 7.0 Hz, 1H), 3.86-3.73 (m, 2H), 3.67 (dd, J=7.4, 3.1 Hz, 1H), 3.57 (td, J=7.0, 3.0 Hz, 1H). LC-MS: m/z=360.0 (M+H⁺)

COMPOUNDS 24 to 38 listed in Table 2 below are prepared using similar procedure described in COMPOUND 7:

TABLE 2

| | | LC-MS: |
|---|---|---|
| COMPOUND | Structure | m/z (M + H⁺) |
| 24 | 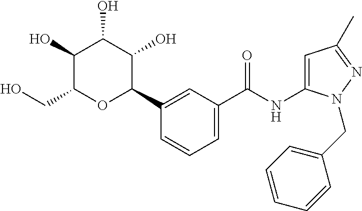 [SMILES: Cc1cc(NC(=O)c2cccc([C@H]3O[C@H](CO)[C@@H](O)[C@H](O)[C@@H]3O)c2)n(Cc2ccccc2)n1] N-(1-benzyl-3-methyl-1H-pyrazol-5-yl)-3-((2R,3S,4R,5S,6R)- 3,4,5-trihydroxy-6-(hydroxymethyl)tetrahydro-2H-pyran-2- yl)benzamide | 454.3 |
| 25 | 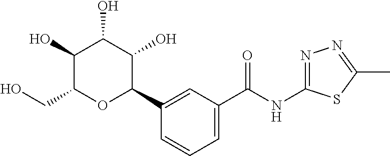 [SMILES: Cc1nnc(NC(=O)c2cccc([C@H]3O[C@H](CO)[C@@H](O)[C@H](O)[C@@H]3O)c2)s1] N-(5-methyl-1,3,4-thiadiazol-2-yl)-3-((2R,3S,4R,5S,6R)-3,4,5- trihydroxy-6-(hydroxymethyl)tetrahydro-2H-pyran-2- yl)benzamide | 382.2 |
| 26 | 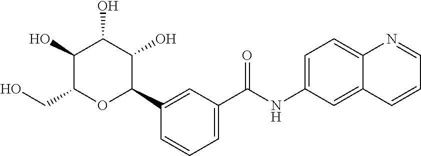 [SMILES: O=C(Nc1ccc2ncccc2c1)c1cccc([C@H]2O[C@H](CO)[C@@H](O)[C@H](O)[C@@H]2O)c1] N-(quinolin-6-yl)-3-((2R,3S,4R,5S,6R)-3,4,5-trihydroxy-6- (hydroxymethyl)tetrahydro-2H-pyran-2-yl)benzamide | 411.5 |
| 27 | 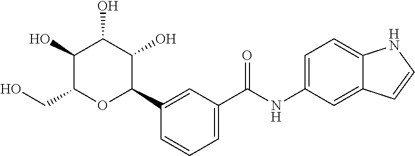 [SMILES: O=C(Nc1ccc2[nH]ccc2c1)c1cccc([C@H]2O[C@H](CO)[C@@H](O)[C@H](O)[C@@H]2O)c1] N-(1H-indol-5-yl)-3-((2R,3S,4R,5S,6R)-3,4,5-trihydroxy-6- (hydroxymethyl)tetrahydro-2H-pyran-2-yl)benzamide | 399.3 |
| 28 | 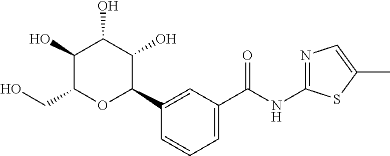 [SMILES: Cc1cnc(NC(=O)c2cccc([C@H]3O[C@H](CO)[C@@H](O)[C@H](O)[C@@H]3O)c2)s1] N-(5-methylthiazol-2-yl)-3-((2R,3S,4R,5S,6R)-3,4,5- trihydroxy-6-(hydroxymethyl)tetrahydro-2H-pyran-2- yl)benzamide | 381.2 |
| 29 | 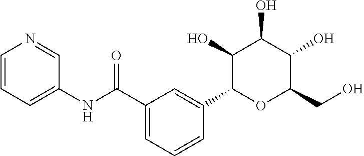 [SMILES: O=C(Nc1cccnc1)c1cccc([C@H]2O[C@H](CO)[C@@H](O)[C@H](O)[C@@H]2O)c1] •TFA salt N-(pyridin-3-yl)-3-((2R,3S,4R,5S,6R)-3,4,5-trihydroxy-6- (hydroxymethyl)tetrahydro-2H-pyran-2-yl)benzamide TFA salt | 361.2 |
| 30 | 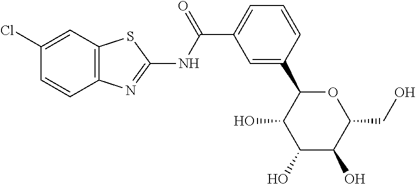 [SMILES: O=C(Nc1nc2ccc(Cl)cc2s1)c1cccc([C@H]2O[C@H](CO)[C@@H](O)[C@H](O)[C@@H]2O)c1] N-(6-chlorobenzo[d]thiazol-2-yl)-3-((2R,3S,4R,5S,6R)-3,4,5- trihydroxy-6-(hydroxymethyl)tetrahydro-2H-pyran-2- yl)benzamide | 451.2 |
| 31 | 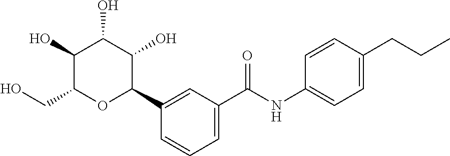 [SMILES: CCCc1ccc(NC(=O)c2cccc([C@H]3O[C@H](CO)[C@@H](O)[C@H](O)[C@@H]3O)c2)cc1] N-(4-propylphenyl)-3-((2R,3S,4R,5S,6R)-3,4,5-trihydroxy-6- (hydroxymethyl)tetrahydro-2H-pyran-2-yl)benzamide | 402.3 |
| 32 | 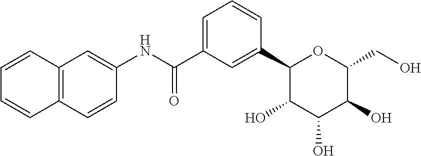 [SMILES: O=C(Nc1ccc2ccccc2c1)c1cccc([C@H]2O[C@H](CO)[C@@H](O)[C@H](O)[C@@H]2O)c1] N-(naphthalen-2-yl)-3-((2R,3S,4R,5S,6R)-3,4,5-trihydroxy-6- (hydroxymethyl)tetrahydro-2H-pyran-2-yl)benzamide | 410.2 |
| 33 | 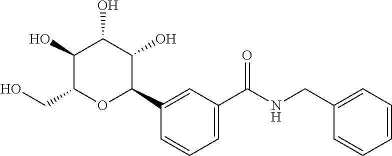 [SMILES: O=C(NCc1ccccc1)c1cccc([C@H]2O[C@H](CO)[C@@H](O)[C@H](O)[C@@H]2O)c1] N-benzyl-3-((2R,3S,4R,5S,6R)-3,4,5-trihydroxy-6- (hydroxymethyl)tetrahydro-2H-pyran-2-yl)benzamide | 374.0 |
| 34 | 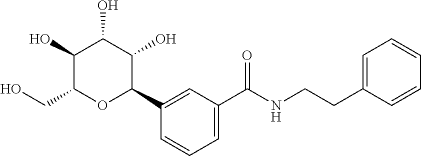 [SMILES: O=C(NCCc1ccccc1)c1cccc([C@H]2O[C@H](CO)[C@@H](O)[C@H](O)[C@@H]2O)c1] N-phenethyl-3-((2R,3S,4R,5S,6R)-3,4,5-trihydroxy-6- (hydroxymethyl)tetrahydro-2H-pyran-2-yl)benzamide | 388.0 |
| 35 | 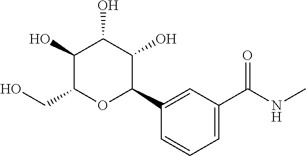 [SMILES: CNC(=O)c1cccc([C@H]2O[C@H](CO)[C@@H](O)[C@H](O)[C@@H]2O)c1] N-methyl-3-((2R,3S,4R,5S,6R)-3,4,5-trihydroxy-6- (hydroxymethyl)tetrahydro-2H-pyran-2-yl)benzamide | 298.2 |
| 36 | 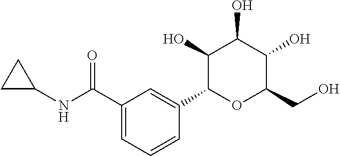 [SMILES: O=C(NC1CC1)c1cccc([C@H]2O[C@H](CO)[C@@H](O)[C@H](O)[C@@H]2O)c1] N-cyclopropyl-3-((2R,3S,4R,5S,6R)-3,4,5-trihydroxy-6- (hydroxymethyl)tetrahydro-2H-pyran-2-yl)benzamide | 324.2 |
| 37 | 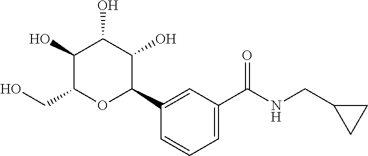 [SMILES: O=C(NCC1CC1)c1cccc([C@H]2O[C@H](CO)[C@@H](O)[C@H](O)[C@@H]2O)c1] N-(cyclopropylmethyl)-3-((2R,3S,4R,5S,6R)-3,4,5-trihydroxy- 6-(hydroxymethyl)tetrahydro-2H-pyran-2-yl)benzamide | 338.0 |
| 38 | 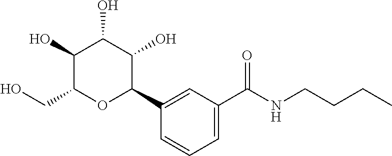 [SMILES: CCCCNC(=O)c1cccc([C@H]2O[C@H](CO)[C@@H](O)[C@H](O)[C@@H]2O)c1] N-butyl-3-((2R,3S,4R,5S,6R)-3,4,5-trihydroxy-6- (hydroxymethyl)tetrahydro-2H-pyran-2-yl)benzamide | 340.0 |

### Example 8

### Preparation of Compound 39

### N-methyl-2′-((2R,3S,4R,5S,6R)-3,4,5-trihydroxy-6-(hydroxymethyl)tetrahydro-2H-pyran-2-yl)-[1,1′-biphenyl]-3-carboxamide

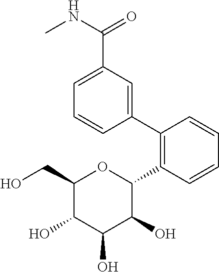 [SMILES: CNC(=O)c1cccc(-c2ccccc2[C@H]2O[C@H](CO)[C@@H](O)[C@H](O)[C@@H]2O)c1]

### Step I: ((2R,3S,4R,5S,6R)-3-acetoxy-4,5-dihydroxy-6-(3′-(methylcarbamoyl)-[1,1′-biphenyl]-2-yl)tetrahydro-2H-pyran-2-yl)methyl acetate

A mixture of INTERMEDIATE E (50 mg, 0.106 mmol), [3-(methylcarbamoyl)phenyl]boronic acid (38 mg, 0.21 mmol) and potassium phosphate (43 mg, 0.32 mmol) in dioxane (530 μL) is degassed by bubbling nitrogen through for 2 mins. PdCl₂(dppf)₂.CH₂CL₂ (7 mg, 0.011 mmol) is added and the mixture is heated in a 4 mL sealed vial at 90° C. for 8 h. The mixture is filtered through a pad of celite and purified directly by reverse phase HPLC to afford the title product (21 mg, 43%).

LC-MS: m/z=458.3 (M+H⁺)

### Step II: Compound 39

((2R,3S,4R,5S,6R)-3-acetoxy-4,5-dihydroxy-6-(3′-(methylcarbamoyl)-[1,1′-biphenyl]-2-yl)tetrahydro-2H-pyran-2-yl)methyl acetate (21 mg, 0.046 mmol) is dissolved in MeOH (0.5 mL) and MeONa/MeOH (4.6 μL of 25% w/v, 0.021 mmol) is added and the reaction mixture is stirred at room temperature for 6 h. The reaction is neutralized by the addition of Amberlite IR120H resin until the pH changed to neutral. The reaction mixture is filtered and the filtrate is evaporated to afford the title compound (18 mg, 40%) as a white solid.

¹H NMR (400 MHz, CD₃OD) δ 7.94 (s, 1H), 7.80 (d, J=7.7 Hz, 1H), 7.76 (d, J=7.6 Hz, 1H), 7.62 (d, J=7.6 Hz, 1H), 7.53 (t, J=7.7 Hz, 1H), 7.45 (t, J=7.6 Hz, 1H), 7.38 (t, J=7.1 Hz, 1H), 7.28 (d, J=7.4 Hz, 1H), 5.02 (d, J=8.6 Hz, 1H), 4.20 (dd, J=8.6, 3.2 Hz, 1H), 3.97-3.92 (m, 1H), 3.83-3.73 (m, 3H), 3.55 (q, J=9.5 Hz, 1H), 2.92 (s, 3H). LC-MS: m/z=374.2 (M+H⁺)

### Example 9

### Preparation of Compound 40

### N³,N⁵-dimethyl-2′-((2R,3S,4R,5S,6R)-3,4,5-trihydroxy-6-(hydroxymethyl)tetrahydro-2H-pyran-2-yl)-[1,1′-biphenyl]-3,5-dicarboxamide

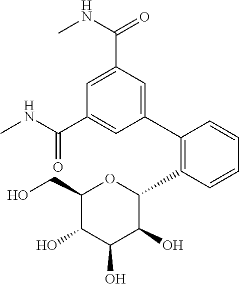 [SMILES: CNC(=O)c1cc(C(=O)NC)cc(-c2ccccc2[C@H]2O[C@H](CO)[C@@H](O)[C@H](O)[C@@H]2O)c1]

The title compound is prepared according to the procedure described for COMPOUND 39 but using N1,N3-dimethyl-5-(4,4,5,5-tetramethyl-1,3,2-dioxaborolan-2-yl)benzene-1,3-dicarboxamide (prepared according to the literature procedure Corinne K. Cusumano, et al. Sci. Transl. Med. 3, 109ra115 (2011)) in Step 1.

¹H NMR (400 MHz, CD₃OD) δ 8.27 (t, J=1.4 Hz, 1H), 8.08 (d, J=1.3 Hz, 2H), 7.78 (d, J=7.6 Hz, 1H), 7.48 (t, J=7.0 Hz, 1H), 7.41 (t, J=7.4 Hz, 1H), 7.33 (d, J=6.5 Hz, 1H), 4.99 (d, J=8.6 Hz, 1H), 4.21 (dd, J=8.6, 3.1 Hz, 1H), 4.01-3.93 (m, 1H), 3.86-3.72 (m, 3H), 3.58-3.46 (m, 1H), 2.94 (s, 6H). LC-MS: m/z=431.2 (M+H⁺)

### Example 10

### Preparation of Compound 41

### (2R,3S,4R,5S,6R)-2-(5-bromo-2-methoxyphenyl)-6-(hydroxymethyl)tetrahydro-2H-pyran-3,4,5-triol

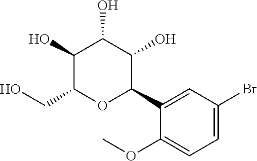 [SMILES: COc1ccc(Br)cc1[C@H]1O[C@H](CO)[C@@H](O)[C@H](O)[C@@H]1O]

To a mixture of [(2R,3R,4S,5S,6R)-3,4,5,6-tetraacetoxytetrahydropyran-2-yl]methyl acetate (40 mg, 0.103 mmol), 1-bromo-4-methoxy-benzene (38 mg, 0.21 mmol) and (2,2,2-trifluoroacetyl)oxysilver (34 mg, 0.16 mmol) in dichloromethane (500 μL) is added tetrachlorostannane in CH₂Cl₂ (308 μL of 1 M, 0.308 mmol) at 0° C. and the reaction mixture is warmed to room temperature and stirred for 15 h. The mixture is diluted with saturated aqueous NaHCO₃ (1 mL), filtered using a phase separator cartridge and washed with CH₂Cl₂ (1 mL). The filtrate is evaporated to afford the crude product (2R,3R,4R,5R,6R)-2-(acetoxymethyl)-6-(5-bromo-2-methoxyphenyl)tetrahydro-2H-pyran-3,4,5-triyltriacetate which is dissolved in methanol (0.5 mL), treated with MeONa/MeOH (6.7 μL of 25% w/v, 0.031 mmol) and stirred at room temperature for 3 h. The reaction mixture is washed with Hex (2×1 mL), the solvent is evaporated and purified directly by reverse phase HPLC to afford the title product (3.2 mg).

¹H NMR (400 MHz, CD₃OD) δ 7.67 (d, J=2.5 Hz, 1H), 7.39 (dd, J=8.8, 2.5 Hz, 1H), 6.92 (d, J=8.8 Hz, 1H), 5.20 (d, J=6.9 Hz, 1H), 4.19 (dd, J=6.9, 3.0 Hz, 1H), 4.09-3.99 (m, 1H), 3.86-3.72 (m, 6H). LC-MS: m/z=371.1, 373.1 (M+Na⁺)

### Example 11

### Preparation of Compound 42

### Methyl 3-(4-methoxy-3-((2R,3S,4R,5S,6R)-3,4,5-trihydroxy-6-(hydroxymethyl)tetrahydro-2H-pyran-2-yl)phenyl)propanoate

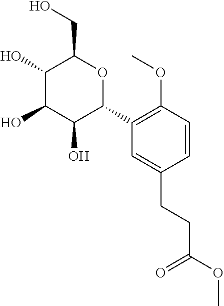 [SMILES: COC(=O)CCc1ccc(OC)c([C@H]2O[C@H](CO)[C@@H](O)[C@H](O)[C@@H]2O)c1]

The title compound is prepared according to the procedure described for COMPOUND 41, but using methyl 3-(3-bromo-4-methoxyphenyl)propanoate as the starting material.

¹H NMR (400 MHz, CD₃OD) δ 7.39 (d, J=2.1 Hz, 1H), 7.12 (dd, J=8.4, 2.3 Hz, 1H), 6.91 (d, J=8.4 Hz, 1H), 5.19 (d, J=5.8 Hz, 1H), 4.34 (dd, J=5.8, 3.1 Hz, 1H), 3.98 (dd, J=11.4, 6.5 Hz, 2H), 3.87-3.76 (m, 5H), 3.74 (dd, J=6.3, 3.1 Hz, 1H), 3.64 (s, 3H), 2.88 (t, J=7.6 Hz, 2H), 2.62 (t, J=7.6 Hz, 2H). LC-MS: m/z=357.2 (M+H⁺)

### Example 12

### Preparation of Compounds 43-47

COMPOUNDS 43-47 are prepared using similar procedure described for INTERMEDIATE A, but using the appropriate boronic acid as starting material.

### Compound 43

### (2R,3S,4R,5S,6R)-2-(hydroxymethyl)-6-(3-phenylphenyl)tetrahydropyran-3,4,5-triol

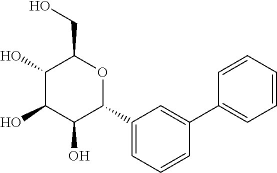 [SMILES: OC[C@H]1O[C@H](c2cccc(-c3ccccc3)c2)[C@@H](O)[C@@H](O)[C@@H]1O]

¹H NMR (400 MHz, CD₃OD) δ 7.76 (s, 1H), 7.63 (dd, 1H), 7.61 (dd, 1H), 7.53 (m, 1H), 7.44-7.39 (m, 2H), 7.33-7.29 (m, 1H), 5.02 (d, 1H), 4.47 (t, 1H), 3.95-3.83 (m, 2H), 3.73 (t, 1H0, 3.63-3.61 (dd, 1H), 3.54-3.51 (m, 1H). LC-MS: m/z=339.2 (M+Na⁺).

### Compound 44

### (2R,3S,4R,5S,6R)-2-(hydroxymethyl)-6-(3-methoxyphenyl)tetrahydropyran-3,4,5-triol

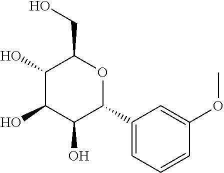 [SMILES: COc1cccc([C@H]2O[C@H](CO)[C@@H](O)[C@H](O)[C@@H]2O)c1]

¹H NMR (400 MHz, CD₃OD) δ 7.27 (t, 1H), 7.07 (s, 1H), 7.00 (d, 1H), 6.82 (dd, 1H), 4.92 (d, 1H), 4.41 (m, 1H), 3.83-3.80 (m, 2H), 3.77 (s, 3H), 3.69 (t, 1H), 3.54 (dd, 1H), 3.46 (m, 1H). LC-MS: m/z=293.2 (M+Na⁺).

### Compound 45

### (2R,3S,4R,5S,6R)-2-(hydroxymethyl)-6-(2-hydroxyphenyl)tetrahydropyran-3,4,5-triol

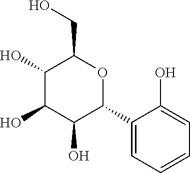 [SMILES: OC[C@H]1O[C@H](c2ccccc2O)[C@@H](O)[C@@H](O)[C@@H]1O]

¹H NMR (400 MHz, CD₃OD) δ 7.32 (d, 1H), 7.14 (t, 1H), 6.85-6.78 (m, 2H), 5.12 (d, 1H), 4.44 (m, 1H), 3.94 (dd, 1H), 3.80-3.73 (m, 3H), 3.62 (m, 1H). LC-MS: m/z=279.2 (M+Na⁺).

### Compound 46

### (2R,3S,4R,5S,6R)-2-(hydroxymethyl)-6-(4-hydroxyphenyl)tetrahydropyran-3,4,5-triol

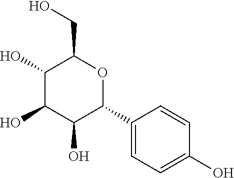 [SMILES: OC[C@H]1O[C@H](c2ccc(O)cc2)[C@@H](O)[C@@H](O)[C@@H]1O]

¹H NMR (400 MHz, CD₃OD) δ 7.26 (d, 2H), 6.77 (d, 2H), 4.88 (m, 2H), 4.38 (t, 1H), 3.78 (m, 1H), 3.72-3.68 (t, 1H), 3.6-3.57 (dd, 1H), 3.43-3.39 (m, 1H). LC-MS: m/z=279.2 (M+Na⁺).

### Compound 47

### (2R,3S,4R,5S,6R)-2-(3-fluorophenyl)-6-(hydroxymethyl)tetrahydropyran-3,4,5-triol

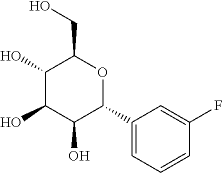 [SMILES: OC[C@H]1O[C@H](c2cccc(F)c2)[C@@H](O)[C@@H](O)[C@@H]1O]

¹H NMR (400 MHz, CD₃OD) δ 7.37 (td, 1H), 7.30-7.19 (m, 2H), 6.99 (tt, 1H), 4.92 (t, 1H), 4.39-4.28 (m, 1H), 3.90-3.76 (m, 2H), 3.76-3.65 (m, 1H), 3.56 (dd, 1H), 3.48 (td, 1H). LC-MS: m/z=281.2 (M+Na⁺).

### Compound 48

### (2R,3S,4R,5S,6R)-2-(hydroxymethyl)-6-[3-[4-(5-methyl-1,3,4-oxadiazol-2-yl)phenyl]phenyl]tetrahydropyran-3,4,5-triol

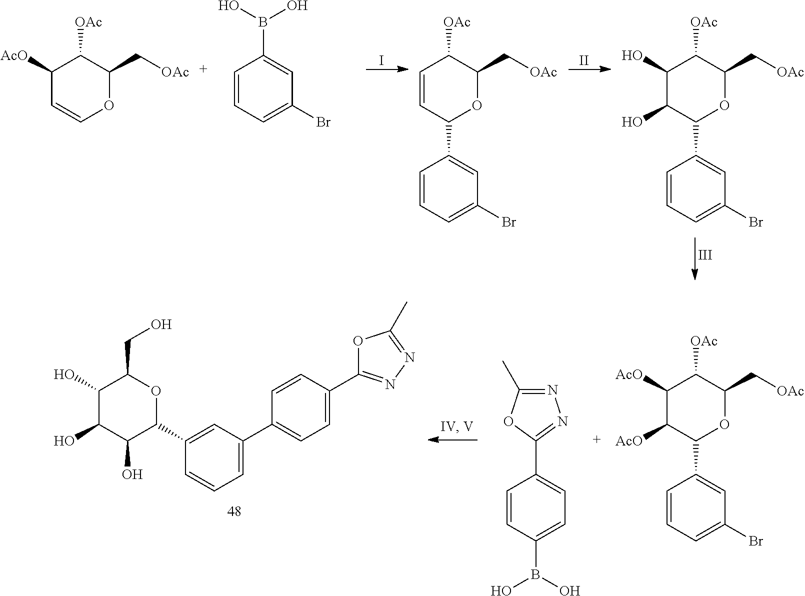

### Step I: ((2R,3S,6S)-3-acetoxy-6-(3-bromophenyl)-3,6-dihydro-2H-pyran-2-yl)methyl acetate

To a solution of [(2R,3S,4R)-3,4-diacetoxy-3,4-dihydro-2H-pyran-2-yl]methyl acetate (34 g, 124.9 mmol) and (3-bromophenyl)boronic acid (55.19 g, 274.8 mmol) in pre-degassed (alternating bubbling of N₂/vacuum×4 times) acetonitrile (340.0 mL) was added diacetoxypalladium (4.207 g, 18.74 mmol) and the reaction mixture was stirred at room temperature for 65 hrs under an inert (N₂) atmosphere. Additional diacetoxypalladium (2.804 g, 12.49 mmol) was added after 23 h. The suspension was filtered over Celite, rinsing with EtOAc. The filtrate was concentrated to dryness, to afford 90 g of a brown oil. The residue was filtered through a pad of Silica Gel (16.5 cm×7 cm, 1.2 L silica) eluting with Hex/EtOAc 50/50 (16 volume), then the filtrate was concentrated to dryness.

The product was purified on a Biotage™ Chromatography in 6 batches (dry loaded on ˜1.5 g silica per g of crude) using 340 g Snap cartridge or 100 g Snap Ultra cartridge and a gradient of 5%-30% EtOAc/Hexanes as the eluent with a flow rate of 100 mL/min or 5 0 mL/ml n (collect at 210 and 220 nm) over 14 CV to afford the title compound (20.0 g, 43.3%). The mixed fractions (2.66 g crude mass) were combined and re-purified by Biotage™ Chromatography (dry loaded) using Snap Ultra 50 g silica gel cartridge and a gradient of 5%-30% EtOAc/Hexanes as the eluent with a flow rate of 50 mL/min over 14 CV to afford additional desired material (1.04 g, 2.2%).

¹H NMR (400 MHz, Methanol-d4) δ 7.61 (dd, J=2.1, 1.3 Hz, 1H), 7.52-7.45 (m, 1H), 7.41-7.35 (m, 1H), 7.30 (t, J=7.8 Hz, 1H), 6.26 (ddd, J=10.4, 3.1, 1.6 Hz, 1H), 5.99 (ddd, J=10.4, 2.9, 2.1 Hz, 1H), 5.36-5.28 (m, 1H), 5.22 (dddd, J=6.8, 2.8, 2.1, 1.5 Hz, 1H), 4.24 (dd, J=12.0, 6.9 Hz, 1H), 4.14 (dd, J=12.0, 3.1 Hz, 1H), 3.79 (td, J=7.0, 3.1 Hz, 1H), 2.08 (s, 3H), 2.06 (s, 3H). LCMS: Mass found for (M+Na)=393.

### Step II: ((2R,3S,4R,5S,6R)-3-acetoxy-6-(3-bromophenyl)-4,5-dihydroxytetrahydro-2H-pyran-2-yl)methyl acetate

To a solution of [(2R,3S,65)-3-acetoxy-6-(3-bromophenyl)-3,6-dihydro-2H-pyran-2-yl]methyl acetate (20.6 g, 55.80 mmol) in THF (618.0 mL)/water (412.0 mL) was added OsO4 (2.5% Wt. in -t-BuOH) (23.84 g, 29.40 mL, 2.344 mmol) and 4-methyl-4-oxido-morpholin-4-ium (19.61 g, 17.35 mL, 167.4 mmol). The mixture was stirred at room temperature for 6 days. The resulting mixture is poured on 2-Me-THF (500 mL) and 25 mL brine. The layers are separated and the aqueous layer is back extracted with 1×500 mL 2-Me-THF. Combined organic layers are washed with once with brine (200 mL), twice with Na₂SO₃ 15% (200 mL) and once again with brine (200 mL). The solution was concentrated to a minimum volume. Upon concentration, the residue turned into a light yellow gelatinous solid. Addition of CH₂Cl₂ (˜250 mL) allowed the product to crystallize out of solution. The suspension was stirred at room temperature for 2 hrs, sonicated for 5 min and stirred again at room temperature for an additional hour. The mixture was then filtered and the solid was rinsed with cold CH₂Cl₂ (2×50 mL) to afford 7.21 g of white-grey solid.

The mother liquors from filtration are concentrated to dryness and the residue purified by flash chromatography to afford additional desired material. A total of 12.1 g (54%) of the title compound is contained.

### Step III: (2R,3R,4R,5R,6R)-2-(acetoxymethyl)-6-(3-bromophenyl)tetrahydro-2H-pyran-3,4,5-triyl triacetate

To a solution of [(2R,3S,4R,5S,6R)-3-acetoxy-6-(3-bromophenyl)-4,5-dihydroxy-tetrahydropyran-2-yl]methyl acetate (12.1 g, 30.01 mmol) in pyridine (28.48 g, 29.12 mL, 360.1 mmol) is added 4-(DIMETHYLAMINO)PYRIDINE (366.6 mg, 3.001 mmol). The reaction mixture is then cooled in an ice bath and acetic anhydride (24.51 g, 22.65 mL, 240.1 mmol) is added dropwise keeping the temperature below 10° C. The resulting mixture is stirred for 20 hours at room temperature. 100 ml of water is poured into reaction mixture and stirred for 5 minutes. 100 ml CH₂Cl₂ is then added and stirred 5 min. Add 200 ml of 1N HCl (pH=4-5) transfer in a separation funnel and cut the phase. The aqueous phase was back-extracted with CH₂Cl₂ (2×50 ml), merge the organic phases. The organic phase is back washed with 200 ml more HCl 1N (pH=1) stir for 10 minutes then cut the phase. The organic phase is then dried with sodium sulfate filter and evaporate to dryness, co-evaporated with heptane (3×100 ml) then dried on the vacuum pump over the weekend to afford [(2R,3R,4R,5R,6R)-3,4,5-triacetoxy-6-(3-bromophenyl)tetrahydropyran-2-yl]methyl acetate (13.04 g, 26.76 mmol, 89.19%) as a beige powder.

¹H NMR (400 MHz, Chloroform-d) δ 7.67 (d, J=1.4 Hz, 1H), 7.52-7.46 (m, 1H), 7.45-7.39 (m, 1H), 7.30 (t, J=7.9 Hz, 1H), 5.88 (t, J=3.3 Hz, 1H), 5.30 (t, J=8.6 Hz, 1H), 5.11 (dd, J=8.8, 3.1 Hz, 1H), 5.06 (d, J=3.5 Hz, 1H), 4.38 (dd, J=12.1, 6.9 Hz, 1H), 4.16 (dd, J=12.1, 2.8 Hz, 1H), 3.82-3.72 (m, 1H), 2.15 (d, J=1.4 Hz, 6H), 2.07 (s, 3H), 2.04 (s, 3H).

### Step IV: (2R,3R,4R,5R,6R)-2-(acetoxymethyl)-6-(4′-(5-methyl-1,3,4-oxadiazol-2-yl)-[1,1′-biphenyl]-3-yl)tetrahydro-2H-pyran-3,4,5-triyl triacetate

In a 1 L 3 necks round bottom flask equipped with a condenser, heating mantle, magnetic stirrer and N₂ inlet is dissolved [(2R,3R,4R,5R,6R)-3,4,5-triacetoxy-6-(3-bromophenyl)tetrahydropyran-2-yl]methyl acetate (18.1 g, 37.14 mmol), [4-(5-methyl-1,3,4-oxadiazol-2-yl)phenyl]boronic acid (12.50 g, 61.28 mmol) and sodium bicarbonate (132.6 mL of 1.4 M, 185.7 mmol) in dioxane (543.0 mL). Pd(PPh3)4 (4.292 g, 3.714 mmol) is then added and the mixture (yellow thick slurry) is stirred at 90° C. for 4 h. The reaction mixture is cooled to room temperature, filtered over celite to remove inorganic salts and the filtrate is concentrated. The residue is dissolved back in 250 mL of EtOAc, adsorb on 50 g of silica gel then purify in two batches on 340 g Snap Ultra cartridge with a gradient from 30-80% EtOAc/Hexanes. The appropriated fractions are merged, and then evaporated to afford the title compound (11.74 g, 56%)

¹H NMR (400 MHz, Chloroform-d) δ 8.13 (d, J=8.4 Hz, 2H), 7.84-7.76 (m, 3H), 7.63 (dd, J=5.8, 2.0 Hz, 1H), 7.54 (dd, J=4.9, 1.7 Hz, 2H), 6.08 (t, J=3.1 Hz, 1H), 5.37 (t, J=9.0 Hz, 1H), 5.20 (dd, J=9.2, 3.0 Hz, 2H), 4.38 (dd, J=12.1, 6.6 Hz, 1H), 4.19-4.06 (m, 1H), 3.83 (ddd, J=9.0, 6.6, 2.6 Hz, 1H), 2.64 (s, 3H), 2.19 (s, 3H), 2.09 (s, 3H), 2.08 (s, 3H), 2.04 (s, 1H), 2.02 (s, 3H), 1.59 (s, 2H), 1.26 (t, J=7.2 Hz, 1H).

### Step V: Compound 48

To a solution of [(2R,3R,4R,5R,6R)-3,4,5-triacetoxy-6-[3-[4-(5-methyl-1,3,4-oxadiazol-2-yl)phenyl]phenyl]tetrahydropyran-2-yl]methyl acetate (11.74 g, 20.72 mmol) in methanol (293.5 mL) is added MeONa (2.239 g, 2.308 mL of 25% w/w, 10.36 mmol). The mixture (yellow sltn) is stirred at room temperature under nitrogen for 1.5 h. Neutralize through a amberlist resine IR-120 (×g) column (pH 8-9 to 4-5) then treated with 130 mg (4 eq., loading of 1.2 mmol/g, assumed 500 ppm residual Pd) of SiliaMetS Thiol. The mixture is stirred at room temperature (1 h) then filtered on a celite pad and evaporated to dryness to yield a yellow solid. The residue is adsorbed on silica gel then purify on a 100 g snap HP cartridge with a gradient from 0-25% MeOH in dichloromethane. Then the appropriated fractions are merge and evaporated to dryness to afford 2.76 g of white solid. The solid is suspended in MeOH (93 mL, 65 vol). The mixture is then stirred at 65° C. until complete dissolution of the product (45 min) The solution is cooled to room temperature then evaporated under light vacuum on rotavap (bath temperature: 40° C.) until the product crashed out (15-20 ml of MeOH left). MTBE 30 mL (20 vol) is added to the mixture and stirred at rt for 1 h. The white solid is then filtered on a Buchner, washed with MTBE and dried on Buchner to afford 1.3135 g. The solid was then dried in vacuum oven at 45° C. for 4 days to afford the totle compound (1.299 g)

¹H NMR (400 MHz, DMSO-d6) δ 8.06 (d, J=8.4 Hz, 2H), 7.91 (d, J=8.5 Hz, 2H), 7.81 (s, 1H), 7.66 (dt, J=6.7, 2.1 Hz, 1H), 7.53-7.45 (m, 2H), 4.86 (d, J=4.7 Hz, 1H), 4.80 (dd, J=11.9, 5.5 Hz, 2H), 4.72-4.63 (m, 2H), 4.11 (td, J=6.0, 3.1 Hz, 1H), 3.68 (td, J=6.7, 4.9 Hz, 2H), 3.58 (q, J=5.7 Hz, 1H), 3.50 (ddt, J=6.3, 5.1, 2.3 Hz, 2H), 3.16 (d, J=5.3 Hz, 0H), 3.07 (s, 0H), 2.60 (s, 3H), 1.10 (s, 0H).

¹H NMR (400 MHz, CD₃OD) δ 8.09 (d, 2H), 7.86 (d, 3H), 7.64-7.61 (m, 1H), 7.520-7.47 (m, 2H), 5.03 (d, 1H), 4.59-4.36 (m, 1H), 3.89-3.81 (m, 2H), 3.74 (t, 1H), 3.63 (dd, 1H), 3.55 (td, 1H), 2.62 (s, 3H). LC-MS: m/z=399.2 (M+H⁺).

### Example 13

### Preparation of Compounds 49-50

COMPOUNDS 48-50 are prepared using similar procedure described in COMPOUND 2, but using the appropriate boronic acid in Step I.

### Compound 49

### 2-methyl-N-[4-[3-[(2R,3S,4R,5S,6R)-3,4,5-trihydroxy-6-(hydroxymethyl)tetrahydropyran-2-yl]phenyl]phenyl]propanamide

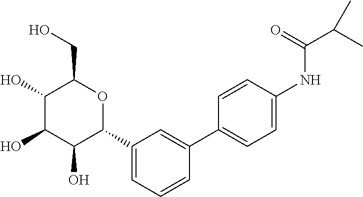 [SMILES: CC(C)C(=O)Nc1ccc(-c2cccc([C@H]3O[C@H](CO)[C@@H](O)[C@H](O)[C@@H]3O)c2)cc1]

¹H NMR (400 MHz, CD₃OD) δ 9.70 (s, 1H), 7.67 (s, 1H), 7.63-7.41 (m, 5H), 7.41-7.22 (m, 2H), 4.93 (d, 1H), 4.39 (t, 1H), 3.75 (d, 2H), 3.68-3.40 (m, 3H), 2.55 (m, 1H), 1.14 (d, 6H). LC-MS: m/z=402.3 (M+H⁺).

### Compound 50

### N-methyl-3-[3-[(2R,3S,4R,5S,6R)-3,4,5-trihydroxy-6-(hydroxymethyl)tetrahydropyran-2-yl]phenyl]benzenesulfonamide

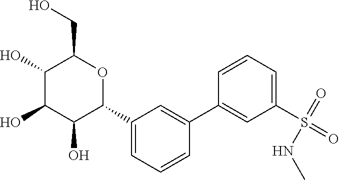 [SMILES: CNS(=O)(=O)c1cccc(-c2cccc([C@H]3O[C@H](CO)[C@@H](O)[C@H](O)[C@@H]3O)c2)c1]

¹H NMR (400 MHz, CD₃OD) δ 8.07 (t, 1H), 7.91 (d, 1H), 7.84-7.75 (m, 2H), 7.70-7.46 (m, 4H), 5.02 (d, 1H), 4.47-4.40 (m, 1H), 3.85 (qd, 2H), 3.74 (t, 1H), 3.63 (dd, 1H), 3.55 (td, 1H), 2.52 (d, 3H). LC-MS: m/z=410.1 (M+H⁺).

Compounds within the scope of the generic Formula X shown below are prepared via two distinct routes. In Route A, compound X is prepared in two steps from the mono-mannoside intermediate Z. In Route B, compound X is obtained as a side product in the last deprotection step leading to bis-mannose type compounds prepared from the bis-mannoside intermediate Y.

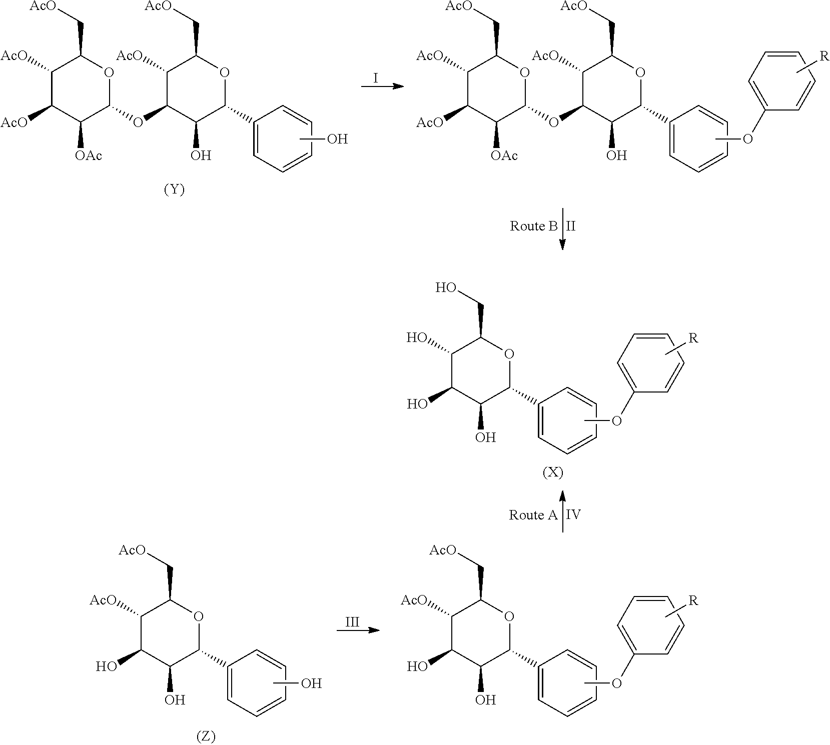

### Example 14

### Preparation of Compound 51 Via Route A

### Dimethyl 5-(3-((2R,3S,4R,5S,6R)-3,4,5-trihydroxy-6-(hydroxymethyl)tetrahydro-2H-pyran-2-yl)phenoxy)isophthalate

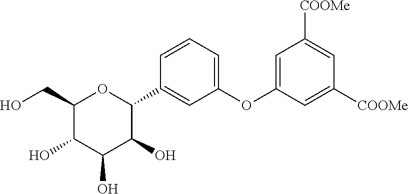 [SMILES: COC(=O)c1cc(Oc2cccc([C@H]3O[C@H](CO)[C@@H](O)[C@H](O)[C@@H]3O)c2)cc(C(=O)OC)c1]

### Step I: Dimethyl 5-(3-((2R,3S,4R,5S,6R)-5-acetoxy-6-(acetoxymethyl)-3,4-dihydroxytetrahydro-2H-pyran-2-yl)phenoxy)isophthalate

To a suspension of INTERMEDIATE A (215 mg, 0.632 mmol) and [3,5-bis(methoxycarbonyl)phenyl]boronic acid (301 mg, 1.26 mmol) in 6.4 mL of CH₂Cl₂ is sequentially added Cu(OAc)₂ (161 mg, 0.884 mmol) and molecular sieves (4 Å, 800 mg). The suspension is stirred at room temperature for 15 min and 2,6-lutidine (366 μL, 3.16 mmol) is added. The reaction mixture is stirred at room temperature for 3 days and is filtered on celite. The filtrate is evaporated to dryness and purified by flash column chromatography on silica gel (0 to 20% MeOH in CH₂Cl₂). The main product is recovered and purified again by reverse phase HPLC to afford the title compound (81 mg, 24%).

### Step II: Compound 51

To a solution of dimethyl 5-(3-((2R,3S,4R,5S,6R)-5-acetoxy-6-(acetoxymethyl)-3,4-dihydroxytetrahydro-2H-pyran-2-yl)phenoxy)isophthalate (77 mg, 0.145 mmol) in 4 mL of MeOH is added NaOMe (25% (w/w) 8.3 μL, 0.036 mmol), under a nitrogen atmosphere. The reaction mixture is stirred at room temperature for 18 hours and filtered over an SPE column (isolute SCX-2, 1 g). The column is washed with methanol and the filtrate is evaporated to dryness. The residue is purified by reverse phase HPLC to afford the title compound (35 mg, 46%).

¹H NMR (400 MHz, DMSO) δ 8.19 (t, J=1.5 Hz, 1H), 7.69 (m, 2H), 7.41 (t, J=7.9 Hz, 1H), 7.27 (d, J=7.7 Hz, 1H), 7.16 (s, 1H), 7.00 (dd, J=7.9, 2.3 Hz, 1H), 4.67 (d, J=5.7 Hz, 1H), 3.97 (dd, J=5.7, 3.0 Hz, 1H), 3.84 (s, 6H), 3.66-3.49 (m, 3H), 3.41 (m, 2H).

### Example 15

### Preparation of Compound 52

### Methyl 4-[3-[(2R,3S,4R,5S,6R)-3,4,5-trihydroxy-6-(hydroxymethyl)tetrahydropyran-2-yl]phenoxy]benzoate

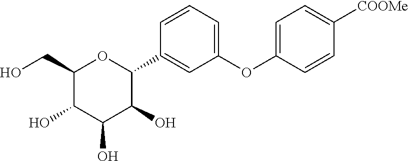 [SMILES: COC(=O)c1ccc(Oc2cccc([C@H]3O[C@H](CO)[C@@H](O)[C@H](O)[C@@H]3O)c2)cc1]

COMPOUND 52 is prepared using the procedure described for COMPOUND 3 but using 4-(methoxycarbonyl)phenyl]boronic acid in the first step.

¹H NMR (400 MHz, DMSO) δ 7.94 (m, 2H), 7.40 (t, J=7.9 Hz, 1H), 7.26 (d, J=7.7 Hz, 1H), 7.14 (s, 1H), 7.02 (m, 3H), 4.66 (d, J=5.7 Hz, 1H), 3.95 (dd, J=5.7, 3.0 Hz, 1H), 3.80 (s, 3H), 3.57 (m, 3H), 3.41 (m, 2H). LC-MS: m/z=391.2 (M+H⁺)

### Example 16

### Preparation of Compound 53

### dimethyl 5-(4-((2R,3S,4R,5S,6R)-3,4,5-trihydroxy-6-(hydroxymethyl)tetrahydro-2H-pyran-2-yl)phenoxy)isophthalate

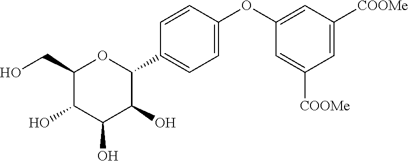 [SMILES: COC(=O)c1cc(Oc2ccc([C@H]3O[C@H](CO)[C@@H](O)[C@H](O)[C@@H]3O)cc2)cc(C(=O)OC)c1]

COMPOUND 53 is prepared using similar procedure described for COMPOUND 3 but using [(2R,3S,4R,5S,6R)-3-acetoxy-4,5-dihydroxy-6-(4-hydroxyphenyl)tetrahydropyran-2-yl]methyl acetate (INTERMEDIATE L, Step II) and 3,5-bis(methoxycarbonyl)phenylboronic acid in the first step.

¹H NMR (400 MHz, DMSO) δ 8.15 (t, J=1.5 Hz, 1H), 7.65 (dd, J=6.5, 1.5 Hz, 2H), 7.43 (d, J=8.5 Hz, 2H), 7.06 (t, J=8.5 Hz, 2H), 4.64 (d, J=6.0 Hz, 1H), 3.94 (dd, J=6.0, 3.0 Hz, 1H), 3.80 (s, 6H), 3.70-3.49 (m, 3H), 3.49-3.33 (m, 2H). LCMS (M+1): 449.3

### Example 17

### Preparation of Compound 54

### methyl 4-(4-((2R,3S,4R,5S,6R)-3,4,5-trihydroxy-6-(hydroxymethyl)tetrahydro-2H-pyran-2-yl)phenoxy)benzoate

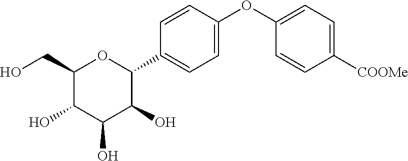 [SMILES: COC(=O)c1ccc(Oc2ccc([C@H]3O[C@H](CO)[C@@H](O)[C@H](O)[C@@H]3O)cc2)cc1]

COMPOUND 54 is prepared using similar procedure described for COMPOUND 3 but using [(2R,3S,4R,5S,6R)-3-acetoxy-4,5-dihydroxy-6-(4-hydroxyphenyl)tetrahydropyran-2-yl]methyl acetate (INTERMEDIATE L, Step II) and 4-(methoxycarbonyl)phenylboronic acid in the first step.

¹H NMR (400 MHz, DMSO) δ 7.94 (m, 1H), 7.46 (d, J=8.6 Hz, 1H), 7.08 (d, J=8.6 Hz, 1H), 7.02 (m, 1H), 4.81 (m, 2H), 4.67 (d, J=5.8 Hz, 1H), 4.62 (d, J=6.0 Hz, 1H), 4.56 (m, 1H), 3.99 (m, 1H), 3.80 (s, 3H), 3.61 (m, 3H), 3.44 (m, 2H), 3.31 (s, 1H). LCMS (M+1): 391.3

### Example 18

### Preparation of Compound 55

### methyl 2-(4-(4-((2R,3S,4R,5S,6R)-3,4,5-trihydroxy-6-(hydroxymethyl)tetrahydro-2H-pyran-2-yl)phenoxy)phenyl)acetate

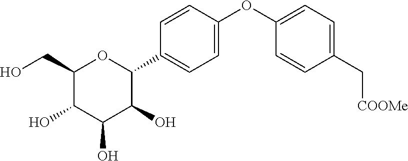 [SMILES: COC(=O)Cc1ccc(Oc2ccc([C@H]3O[C@H](CO)[C@@H](O)[C@H](O)[C@@H]3O)cc2)cc1]

COMPOUND 55 is prepared using the procedure described for COMPOUND 3 but using [(2R,3S,4R,5S,6R)-3-acetoxy-4,5-dihydroxy-6-(4-hydroxyphenyl)tetrahydropyran-2-yl]methyl acetate (INTERMEDIATE L, Step II) and 4-(2-methoxy-2-oxoethyl)phenylboronic acid in the first step.

¹H NMR (400 MHz, DMSO) δ 7.38 (d, J=8.5 Hz, 2H), 7.24 (d, J=8.6 Hz, 2H), 6.93 (m, 4H), 4.65 (d, J=5.6 Hz, 1H), 3.99 (dd, J=5.6, 3.0 Hz, 1H), 3.64 (s, 2H), 3.60 (m, 2H), 3.59 (s, 3H), 3.53 (m, 1H), 3.44 (m, 1H), 3.39 (m, 1H). LCMS (M+1): 405.2

### Example 19

### Preparation of Compound 56

### Methyl 3-(4-((2R,3S,4R,5S,6R)-3,4,5-trihydroxy-6-(hydroxymethyl)tetrahydro-2H-pyran-2-yl)phenoxy)benzoate

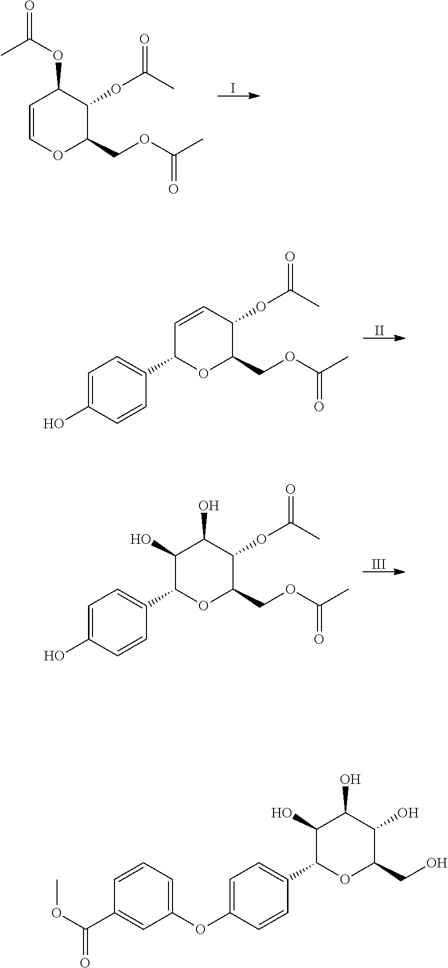

### Step I: ((2R,3S,6S)-3-acetoxy-6-(4-hydroxyphenyl)-3,6-dihydro-2H-pyran-2-yl)methyl acetate

A solution of (2R,3S,4R)-2-(acetoxymethyl)-3,4-dihydro-2H-pyran-3,4-diyl diacetate (9.869 g, 36.25 mmol), 4-(hydroxyphenylboronic acid (5.00 g, 36.3 mmol) and palladium acetate (1.221 g, 5.438 mol) in 50 mL of CH₃CN is stirred at room temperature for 2 days. The mixture is filtered on celite and the filtrate is concentrated to dryness. The residue is purified by chromatography on silica gel (5% to 80% EtOAc in Hex) to afford the title compound (6.03 g, 54%).

### Step II: ((2R,3S,4R,5S,6R)-3-acetoxy-4,5-dihydroxy-6-(4-hydroxyphenyl)tetrahydro-2H-pyran-2-yl)methyl acetate

To a suspension of ((2R,3S,6S)-3-acetoxy-6-(4-hydroxyphenyl)-3,6-dihydro-2H-pyran-2-yl)methyl acetate (6.01 g, 0.591 mmol) in THF (36 mL) and water (24 mL) is added methanesulfonamide (2.81 g, 29.5 mmol), OsO₄ (7.4 mL of a 2.5% (w/w) solution in t-BuOH, 0.591 mmol) and NMO (4.613 g, 39.4 mmol). The mixture is stirred at room temperature for 24 h. The mixture is treated with 40 mL of Na₂S₂O₃ (1M in water) and the product is extracted with EtOAc (3×40 mL). The combined organic layers are washed with brine (15 mL) and dried over Na₂SO₄. The resulting solution is evaporated to dryness and the residue is purified by chromatography on silica gel (0% to 20% MeOH in CH₂Cl₂) to give the title compound (4.447 g, 66%).

### Step III: Compound 56

To a suspension of ((2R,3S,4R,5S,6R)-3-acetoxy-4,5-dihydroxy-6-(4-hydroxyphenyl)tetrahydro-2H-pyran-2-yl)methyl acetate (100 mg, 0.294 mmol) and (3-(methoxycarbonyl)phenyl)boronic acid (106 mg, 0.588 mmol) in 6 mL of CH₂Cl₂ is sequentially added Cu(OAc)₂ (74 mg, 0.411 mmol) and molecular sieves (4 Å, 400 mg). The suspension is stirred at room temperature for 15 minutes and 2,6-lutidine (170 μL, 1.47 mmol) is added. The reaction mixture is stirred at room temperature for 2 days and is filtered on an SPE column (isolute SCX-2, 1 g). The filtrate is evaporated to dryness. To the residue dissolved in methanol (3 mL) is added NaOMe (17 μL of 25% (w/w) solution, 0.073 mmol). The reaction mixture is stirred at room temperature 18 h and filtered over an SPE column (isolute SCX-2, 1 g). The column is washed with MeOH and the filtrate is evaporated to dryness. The residue is purified by reverse phase HPLC to give the title compound (32 mg, 23%).

¹H NMR (400 MHz, DMSO) δ 7.69 (m, 1H), 7.52 (t, J=8.0 Hz, 1H), 7.43 (m, 2H), 7.32 (m, 2H), 7.00 (m, 2H), 4.80 (dd, J=9.8, 4.9 Hz, 2H), 4.66 (d, J=5.8 Hz, 1H), 4.61 (d, J=6.2 Hz, 1H), 4.56 (t, J=5.8 Hz, 1H), 3.99 (m, 1H), 3.79 (s, 3H), 3.63 (m, 2H), 3.56 (m, 1H), 3.44 (m, 2H). LC-MS: m/z=391.2 (M+H⁺)

### Example 20

### Preparation of Compound 57 Via Route B

### (2R,3S,4R,5S,6R)-2-[3-(4-fluorophenoxy)phenyl]-6-(hydroxymethyl)tetrahydropyran-3,4,5-triol

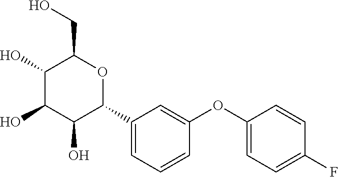 [SMILES: OC[C@H]1O[C@H](c2cccc(Oc3ccc(F)cc3)c2)[C@@H](O)[C@@H](O)[C@@H]1O]

### Step I: [(2R,3R,4R,5R)-3-acetoxy-6-[3-[tert-butyl(dimethyl)silyl]oxyphenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate

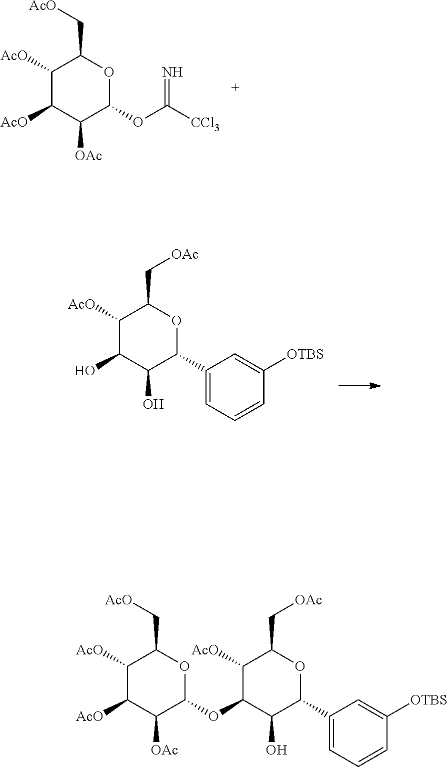

To a stirred solution of [(2R,3R,4S,5S,6R)-3,4,5-triacetoxy-6-(2,2,2-trichloroethanimidoyl)oxy-tetrahydropyran-2-yl]methyl acetate (300 mg, 0.518 mmol) and [(2R,3S,4R,5S)-3-acetoxy-6-[3-[tert-butyl(dimethyl)silyl]oxyphenyl]-4,5-dihydroxy-tetrahydropyran-2-yl]methyl acetate (259 mg, 0.569 mmol) in CH₂Cl₂ (10 mL) is added 4 A MS (1.00 g), stirred at room temperature for 30 min. After cooling to −40° C., freshly opened trimethylsilyl trifluoromethanesulfonate (9.4 μL, 0.052 mmol) is added dropwise. The mixture is stirred at −40° C. and slowly warmed up to −10° C. in 2 h. Then Et₃N (72 μL, 0.52 mmol) is added. After removal of the cooling bath, the mixture is warmed to room temperature, filtered off to remove the molecular sieves, and concentrated to dryness. The residue is purified on Biotage™ SNAP 50 g silica gel cartridge using MeOH/CH₂Cl₂ (0 to 5% in 20 CV) to provide an inseparable mixture containing the title compound (375 mg), which is used directly in the next step without further purification.

LC-MS: m/z=808 (M+Na+).

### Step II: [(2R,3R,4R,5R)-3-acetoxy-5-hydroxy-6-(3-hydroxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate

To a stirred solution of the mixture containing [(2R,3R,4R,5R)-3-acetoxy-6-[3-[tert-butyl(dimethyl)silyl]oxyphenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate (375 mg, 0.478 mmol) in THF (4 mL) are added acetic acid (41 μL, 0.72 mmol) and 1M TBAF/THF (1.43 mL, 1.43 mmol). The mixture is stirred at room temperature for 30 min. It is then diluted EtOAc (30 mL), washed with water (20 mL) and brine (20 mL) consecutively, dried over Na2SO4, concentrated to dryness. The residue is purified on Biotage™ SNAP 25 g silica gel cartridge using CH₂Cl₂/MeOH (0-6%) in 20 column volume to obtain an inseparable mixture (230 mg), containing the title compound which is used directly in the next step without further purification.

### Step III: [(2R,3R,4R,5R,6R)-3-acetoxy-6-[3-(4-fluorophenoxy)phenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate

To a solution of [(2R,3R,4R,5R,6R)-3-acetoxy-5-hydroxy-6-(3-hydroxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate (68 mg, 0.101 mmol) and (4-fluorophenyl)boronic acid (28 mg, 0.203 mmol) in CH₂Cl₂ (3.4 mL) are added dried 4 A molecular sieves (400 mg) and Cu(OAc)₂ (26 mg, 0.142 mmol). After stirring for 10 minutes, 2,6-lutidine (59 mL, 0.51 mmol) is added to the mixture. The reaction mixture is stirred at room temperature for 2 days. After filtration of molecular sieves on celite, the filtrate is concentrated and purified on 10 g SNAP Biotage™ using CH₂Cl₂/MeOH (0 to 5%) in 20 CV to afford a mixture (33 mg) containing the title compound which is used without further purification.

LC-MS: m/z=788 (M+Na⁺).

### Step IV: Compound 57

To a solution of [(2R,3R,4R,5R,6R)-3-acetoxy-6-[3-(4-fluorophenoxy)phenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate (33 mg, 0.0432 mmol) in MeOH (660 mL) is added sodium methoxide/25% in MeOH (8.6 μL of 1 M, 0.0086 mmol). After 2 h, the reaction mixture is passed through an Isolute SCX-2 SPE (2 g) column (pre-wetted with MeOH), washing 2 times with 5 mL of MeOH. The filtrate is evaporated to dryness and purified by reverse phase HPLC to afford the title compound (4 mg) as a white powder.

¹H NMR (400 MHz, CD₃OD) δ 7.26 (t, 1H), 7.11 (d, 1H), 7.05-6.96 (m, 3H), 6.91 (ddd, 2H), 6.78 (dd, 1H), 4.82 (m, 1H), 4.24 (t, 1H), 3.77-3.57 (m, 3H), 3.48 (dd, 1H), 3.43-3.29 (m, 1H). LC-MS: m/z=373.2 (M+Na⁺).

### Example 21

### Preparation of Compounds 58-60

COMPOUNDS 58-60 are prepared according to similar procedure described for COMPOUND 3 but using the appropriate boronic acids:

### Compound 58

### (2R,3S,4R,5S,6R)-2-[3-(3,5-dimethylphenoxy)phenyl]-6-(hydroxymethyl)tetrahydropyran-3,4,5-triol

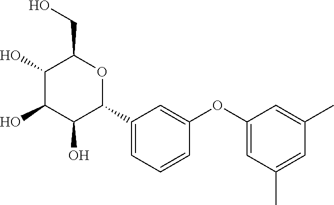 [SMILES: Cc1cc(C)cc(Oc2cccc([C@H]3O[C@H](CO)[C@@H](O)[C@H](O)[C@@H]3O)c2)c1]

¹H NMR (400 MHz, CD₃OD) δ 7.24 (t, 1H), 7.10 (d, 1H), 7.01 (s, 1H), 6.77 (dd, 1H), 6.67 (s, 1H), 6.50 (s, 2H), 4.79 (m, 1H), 4.33 (dd, 1H), 3.73 (dd, 1H), 3.69-3.60 (m, 2H), 3.48 (dd, 1H), 3.41-3.31 (m, 1H), 2.16 (s, 6H). LC-MS: m/z=361.3 (M+H⁺).

### Compound 59

### (2R,3S,4R,5S,6R)-2-(hydroxymethyl)-6-(3-phenoxyphenyl)tetrahydropyran-3,4,5-triol

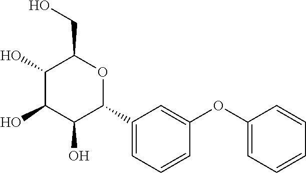 [SMILES: OC[C@H]1O[C@H](c2cccc(Oc3ccccc3)c2)[C@@H](O)[C@@H](O)[C@@H]1O]

¹H NMR (400 MHz, cd3od) δ 7.30-7.19 (m, 3H), 7.13 (d, 1H), 7.02 (dd, 2H), 6.93-6.83 (m, 2H), 6.80 (dd, 1H), 4.8 (m, 1H), 4.25 (t, 1H), 3.77-3.57 (m, 3H), 3.49 (dd, 1H), 3.43-3.32 (m, 1H). LC-MS: m/z=333.2 (M+H⁺).

### Compound 60

### (2R,3S,4R,5S,6R)-2-(hydroxymethyl)-6-[3-[4-(5-methyl-1,3,4-oxadiazol-2-yl)phenoxy]phenyl]tetrahydropyran-3,4,5-triol

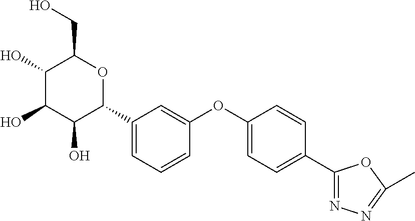 [SMILES: Cc1nnc(-c2ccc(Oc3cccc([C@H]4O[C@H](CO)[C@@H](O)[C@H](O)[C@@H]4O)c3)cc2)o1]

1H NMR (400 MHz, CD₃OD) δ 7.90 (m, 2H), 7.35 (t, 1H), 7.25 (d, 1H), 7.16 (s, 1H), 7.08-6.98 (m, 2H), 6.94 (d, 1H), 4.86 (d, 1H), 4.52 (s, 1H), 4.39-3.97 (m, 1H), 3.79-3.41 (m, 4H), 2.51 (s, 3H). LC-MS: m/z=415.2 (M+H⁺).

### Example 22

### Preparation of Compound 61

### (2R,3S,4R,5S,6R)-2-(3-hydroxy-4-methoxy-phenyl)-6-(hydroxymethyl)tetrahydropyran-3,4,5-triol

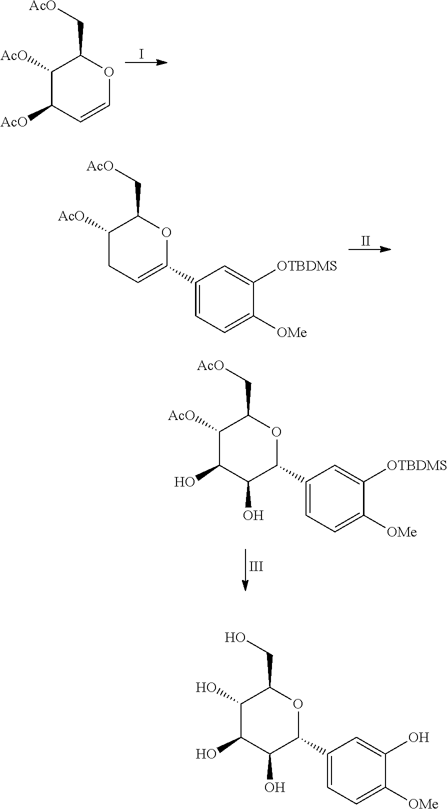

### Step I: [(2R,3S,6S)-3-acetoxy-6-[3-[tert-butyl(dimethyl)silyl]oxy-4-methoxy-phenyl]-3,6-dihydro-2H-pyran-2-yl]methyl acetate

To a solution of commercially available [(2R,3S,4R)-3,4-diacetoxy-3,4-dihydro-2H-pyran-2-yl]methyl acetate (2.00 g, 7.34 mmol) in 35 mL of CH₃CN are added [3-[tert-butyl(dimethyl)silyl]oxy-4-methoxy-phenyl]boronic acid (2.073 g, 7.34 mmol) and Pd(OAc)2 (247 mg, 1.10 mmol). The mixture is stirred at room temperature overnight and then to it are added another batch of Pd(OAc)2 (247 mg, 1.10 mmol) and [3-[tert-butyl(dimethyl)silyl]oxy-4-methoxy-phenyl]boronic acid (2.073 g, 7.35 mmol). It is then stirred at room temperature overnight again. The mixture is diluted with 30 mL of CH₂Cl₂ and filtered over a pad of celite. The filtrate is concentrated and the residue is purified on a Biotage™ (100 g silica gel cartridge) using a gradient of Hex/EtOAc (0-15%) in 20 column volume to afford the title compound (2.00 g, 60% yield) as a yellow oil.

¹H NMR (400 MHz, CDCl₃) δ 6.87-6.72 (m, 2H), 6.67 (d, 1H), 6.05-5.90 (m, 1H), 5.90-5.71 (m, 1H), 5.26-5.02 (m, 2H), 4.09 (dd, 1H), 4.02-3.81 (m, 2H), 3.67 (s, 3H), 1.92 (m, 6H), 0.84 (d, 9H), 0.00 (d, 6H).

### Step II: [(2R,3S,4R,5S,6R)-3-acetoxy-6-[3-[tert-butyl(dimethyl)silyl]oxy-4-methoxy-phenyl]-4,5-dihydroxy-tetrahydropyran-2-yl]methyl acetate

To a solution of [(2R,3S,6S)-3-acetoxy-6-[3-[tert-butyl(dimethyl)silyl]oxy-4-methoxy-phenyl]-3,6-dihydro-2H-pyran-2-yl]methyl acetate (1.85 g, 4.10 mmol) in water (7.4 ml)/t-BuOH (7.4 ml) are added methanesulfonamide (586 mg, 6.16 mmol), 2.5% OsO4/t-BuOH (1.29 mL, 0.10 mmol), NMO (962 mg, 8.21 mmol) and 2,6-lutidine (476 μL, 4.10 mmol). The mixture is stirred at room temperature for 24 h, quenched with 15% sodium bisulfate (15 mL) and diluted with EtOAc. The aqueous phase is separated, washed with water and brine, dried over Na2SO4. After removal of the solvent under reduced pressure, the residue is purified on a Biotage™ SNAP (50 g silica gel cartridge) using a gradient of CH₂Cl₂/MeOH (0-8%) in 20 CV to afford the title compound (1.38 g, 69% yield).

¹H NMR (400 MHz, CD₃OD) δ 6.97-6.34 (m, 3H), 4.95-4.86 (m, 1H), 4.68 (d, 1H), 4.50 (dd, 1H), 4.03 (dd, 1H), 3.89 (dd, 1H), 3.68 (dd, 2H), 3.64 (d, 3H), 1.90 (d, 6H), 1.10-0.68 (m, 9H), 0.00 (d, 6H).

### Step III: Compound 61

To a solution of [(2R,3S,4R,5S,6R)-3-acetoxy-6-[3-[tert-butyl(dimethyl)silyl]oxy-4-methoxy-phenyl]-4,5-dihydroxy-tetrahydropyran-2-yl]methyl acetate (50 mg, 0.103 mmol) in MeOH (1.5 mL) is added a 1M solution of MeONa in MeOH (2.3 μL, 0.0103 mmol). The mixture is stirred at room temperature for 30 min and then to it is added 1M TBAF/THF (103 μL of 1 M, 0.103 mmol). The mixture is stirred at room temperature overnight. Then it is passed through an Isolute SCX-2 SPE (15 mL, 2 g) column (pre-wetted with MeOH), washed twice with 5 mL of MeOH. The filtrate is evaporated to dryness and purified by reverse phase HPLC to afford the title compound (19 mg, 62%).

¹H NMR (400 MHz, CD₃OD) δ 7.24-6.46 (m, 3H), 4.38 (t, 1H), 3.82 (s, 3H), 3.79 (dd, 2H), 3.76-3.66 (m, 1H), 3.58 (dd, 1H), 3.47-3.39 (m, 1H). LC-MS: m/z=573.3 (dimer+H⁺).

### Example 23

### Preparation of Compound 62

### Dimethyl 3′-((2R,3S,4R,5S,6R)-3,4,5-trihydroxy-6-(hydroxymethyl)tetrahydro-2H-pyran-2-yl)-[1,1′-biphenyl]-3,5-dicarboxylate

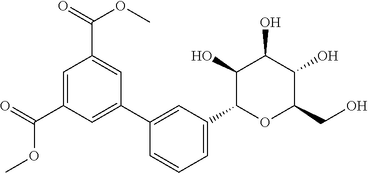 [SMILES: COC(=O)c1cc(C(=O)OC)cc(-c2cccc([C@H]3O[C@H](CO)[C@@H](O)[C@H](O)[C@@H]3O)c2)c1]

To a suspension of INTERMEDIATE B (222 mg, 0.470 mmol) from (3,5-bis(methoxycarbonyl)phenyl)boronic acid (168 mg, 0.705 mmol) and Pd(PPh₃)₄ (54 mg, 0.047 mmol) in 2 mL of dioxane and is added aqueous NaHCO₃ (1.57 mL of 1.2 M solution, 1.88 mmol) under a nitrogen atmosphere. The reaction mixture is heated at 95° C. for 18 hours, cooled to room temperature, and filtered on celite. The filter cake is washed with methanol and the filtrate is evaporated. The residue is dissolved in methanol (2 mL) and MeONa (27 μL of 25% (w/w) solution, 0.118 mmol) is added. The reaction mixture is stirred at room temperature for 18 hours and filtered over an SPE column (isolute SCX-2, 1 g). The column is washed with methanol and the filtrate is evaporated to dryness. The residue is purified by reverse phase HPLC to afford the title compound (85 mg, 38%).

¹H NMR (400 MHz, DMSO) δ 8.44 (t, 1.5 Hz, 1H), 8.39 (d, J=1.6 Hz, 2H), 7.75 (s, 1H), 7.62 (m, 1H), 7.48 (d, J=4.8 Hz, 2H), 4.87 (d, J=4.5 Hz, 1H), 4.82 (d, J=5.1 Hz, 1H), 4.74 (d, J=6.3 Hz, 1H), 4.66 (d, J=6.5 Hz, 1H), 4.59 (t, J=5.7 Hz, 1H), 4.01 (m, 1H), 3.90 (s, 6H), 3.71 (m, 1H), 3.59 (m, 2H), 3.50 (m, 2H). LC-MS: m/z=433.3 (M+H⁺)

### Example 24

### Preparation of Compound 63

### N³,N⁵-dimethyl-3′-((2R,3S,4R,5S,6R)-3,4,5-trihydroxy-6-(hydroxymethyl)tetrahydro-2H-pyran-2-yl)-[1,1′-biphenyl]-3,5-dicarboxamide

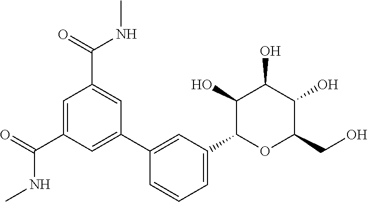 [SMILES: CNC(=O)c1cc(C(=O)NC)cc(-c2cccc([C@H]3O[C@H](CO)[C@@H](O)[C@H](O)[C@@H]3O)c2)c1]

### Step I: 3′-((2R,3S,4R,5S,6R)-3,4,5-trihydroxy-6-(hydroxymethyl)tetrahydro-2H-pyran-2-yl)-[1,1′-biphenyl]-3,5-dicarboxylic acid

To a solution of dimethyl 3′-((2R,3S,4R,5S,6R)-3,4,5-trihydroxy-6-(hydroxymethyl)tetrahydro-2H-pyran-2-yl)-[1,1′-biphenyl]-3,5-dicarboxylate (73 mg, 0.168 mmol) in 1.5 mL of THF and 1.5 mL of water is added LiOH (hydrate, 35 mg, 0.840 mmol) under a nitrogen atmosphere. The reaction mixture is stirred at room temperature for 6 hours, treated with 4M HCl (0.21 mL) and concentrated to dryness. The residue is purified by reverse phase HPLC to afford the title compound (56 mg, 82%).

### Step II: Compound 63

To a solution of 3′-((2R,3S,4R,5S,6R)-3,4,5-trihydroxy-6-(hydroxymethyl)tetrahydro-2H-pyran-2-yl)-[1,1′-biphenyl]-3,5-dicarboxylic acid (50 mg, 0.124 mmol) in 2 mL of DMF is sequentially added methylamine (2M in THF, 155 mL, 0.309 mmol), HATU (118 mg, 0.309 mmol) and DIPEA (65 mL, 0.371 mmol) under nitrogen atmosphere. The reaction mixture is stirred at room temperature for 18 hours and filtered over an SPE column (isolute SCX-2, 1 g). The column is washed with methanol and the filtrate is evaporated to dryness. The residue is purified by reverse phase HPLC to afford the title compound (20 mg, 33%).

¹H NMR (400 MHz, DMSO) δ 8.60 (m, 2H), 8.21 (m, 1H), 8.15 (m, 2H), 7.73 (s, 1H), 7.62 (m, 1H), 7.43 (m, 2H), 4.73 (d, J=5.8 Hz, 1H), 4.04 (dd, J=5.8, 3.0 Hz, 1H), 3.62 (m, 2H), 3.52 (m, 1H), 3.43 (m, 2H), 2.76 (d, J=4.5 Hz, 6H). LC-MS: m/z=431.3 (M+H⁺)

### Example 25

### Preparation of Compound 64

### N1,N3-dimethyl-5-(3-((2R,3S,4R,5S,6R)-3,4,5-trihydroxy-6-(hydroxymethyl)tetrahydro-2H-pyran-2-yl)phenoxy)isophthalamide

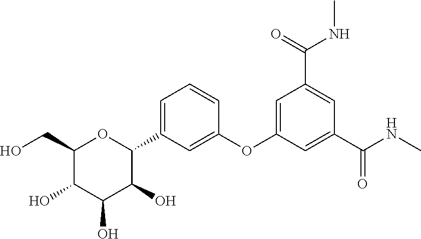 [SMILES: CNC(=O)c1cc(Oc2cccc([C@H]3O[C@H](CO)[C@@H](O)[C@H](O)[C@@H]3O)c2)cc(C(=O)NC)c1]

COMPOUND 64 is prepared according to similar procedure described in Steps I and II of EXAMPLE 24.

¹H NMR (400 MHz, DMSO) δ 8.51 (m, 2H), 8.00 (t, J=1.4 Hz, 1H), 7.50 (dd, J=4.6, 1.4 Hz, 2H), 7.33 (m, 1H), 7.18 (d, J=7.8 Hz, 1H), 7.07 (s, 1H), 6.87 (dd, J=8.0, 2.3 Hz, 1H), 4.63 (d, J=5.7 Hz, 1H), 3.92 (dd, J=5.5, 3.0 Hz, 1H), 3.62-3.32 (m, 5H), 2.70 (d, J=4.5 Hz, 6H). LCMS (M+1): 447.3

### Example 26

### Preparation of Compound 65

### Methyl 2-(3′-((2R,3S,4R,5S,6R)-3,4,5-trihydroxy-6-(hydroxymethyl)tetrahydro-2H-pyran-2-yl)-[1,1′-biphenyl]-3-yl)acetate

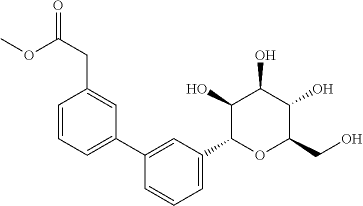 [SMILES: COC(=O)Cc1cccc(-c2cccc([C@H]3O[C@H](CO)[C@@H](O)[C@H](O)[C@@H]3O)c2)c1]

To a suspension of INTERMEDIATE B (100 mg, 0.212 mmol), (3-(2-methoxy-2-oxoethyl)phenyl)boronic acid (62 mg, 0.317 mmol) and Pd(PPh₃)₄ (24 mg, 0.021 mmol) in 2 mL of dioxane and is added aqueous NaHCO₃ (0.71 mL of 1.2 M solution, 0.847 mmol) under nitrogen atmosphere. The reaction mixture is heated at 95° C. for 18 hours, cooled to room temperature, and filtered on celite. The filter cake is washed with methanol and the filtrate is evaporated. The residue is dissolved in methanol (2 mL) and MeONa (12 μL of 25% (w/w) solution, 0.053 mmol) is added. The reaction mixture is stirred at room temperature 18 hours and filtered over an SPE column (isolute SCX-2, 1 g). The column is washed with methanol and the filtrate is evaporated to dryness. The residue is purified by reverse phase HPLC to afford the title compound (31 mg, 33%).

¹H NMR (400 MHz, DMSO) δ 7.67 (s, 1H), 7.52 (m, 3H), 7.40 (m, 3H), 7.24 (d, J=7.6 Hz, 1H), 4.74 (d, J=5.5 Hz, 1H), 4.50 (broad s, 4H), 4.08 (dd, J=5.5, 3.0 Hz, 1H), 3.74 (s, 2H), 3.64 (m, 2H), 3.60 (s, 3H), 3.54 (m, 1H), 3.44 (m, 2H). LC-MS: m/z=389.2 (M+H⁺)

### Example 27

### Preparation of Compound 66

### Methyl 2-(3′-((2R,3S,4R,5S,6R)-3,4,5-trihydroxy-6-(hydroxymethyl)tetrahydro-2H-pyran-2-yl)-[1,1′-biphenyl]-2-yl)acetate

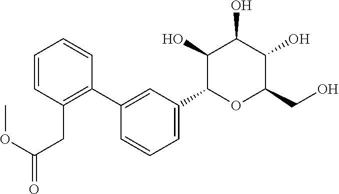 [SMILES: COC(=O)Cc1ccccc1-c1cccc([C@H]2O[C@H](CO)[C@@H](O)[C@H](O)[C@@H]2O)c1]

COMPOUND 66 is prepared according to similar procedure described for COMPOUND 65 but using (2-(2-methoxy-2-oxoethyl)phenyl)boronic acid.

¹H NMR (400 MHz, DMSO) δ 7.38 (m, 2H), 7.32 (m, 3H), 7.28 (s, 1H), 7.21 (m, 1H), 7.12 (m, 1H), 4.71 (d, J=5.3 Hz, 1H), 4.48 (broad s, 4H), 4.05 (dd, J=5.3, 3.1 Hz, 1H), 3.60 (m, 4H), 3.54 (m, 1H), 3.49 (s, 3H), 3.43 (m, 1H), 3.39 (m, 1H). LC-MS: m/z=389.2 (M+H⁺)

### Example 28

### Preparation of Compound 67

### (2R,3S,4R,5S,6R)-2-(4-chloro-3-hydroxyphenyl)-6-(hydroxymethyl)tetrahydro-2H-pyran-3,4,5-triol

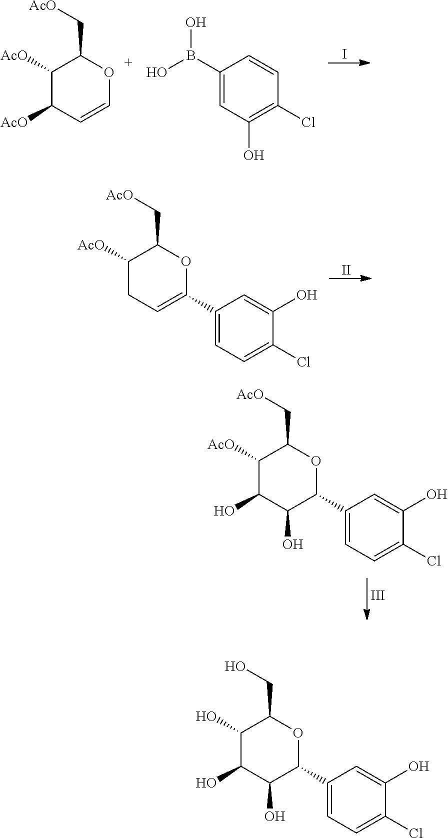

### Step I: ((2R,3S,6S)-3-acetoxy-6-(4-chloro-3-hydroxyphenyl)-3,6-dihydro-2H-pyran-2-yl)methyl acetate

To a solution of [(2R,3S,4R)-3,4-diacetoxy-3,4-dihydro-2H-pyran-2-yl]methyl acetate (0.900 g, 3.31 mmol in 10 mL of CH₃CN are added (4-chloro-3-hydroxy-phenyl)boronic acid (1.140 g, 6.61 mmol) and Pd(OAc)₂ (111 mg, 0.496 mmol). The mixture was stirred at room temperature for 23 h. To the reaction was added another portion of Pd(OAc)₂ (111 mg, 0.496 mmol) and (4-chloro-3-hydroxy-phenyl)boronic acid (0.350 g, 2.03 mmol). It was then stirred at room temperature for 27 h, when complete consumption of the starting material was observed. The mixture was diluted with 10 mL of CH₂Cl₂ and filtered over a pad of celite. The filtrate was concentrated to a black foam (1.50 g). The crude material was purified on a 50 g SNAP silica cartridge, eluting with Hex/EtOAc (0-50%) to give the title compound (328 mg, 28%) as a white foam. LC-MS: m/z=363 (M+Na⁺).

### Step II: ((2R,3S,4R,5S,6R)-3-acetoxy-6-(4-chloro-3-hydroxyphenyl)-4,5-dihydroxytetrahydro-2H-pyran-2-yl)methyl acetate

To a solution of [(2R,3S,65)-3-acetoxy-6-(4-chloro-3-hydroxy-phenyl)-3,6-dihydro-2H-pyran-2-yl]methyl acetate (328 mg, 0.963 mmol) in water (5.9 mL)/t-BuOH (5.9 mL) are added methanesulfonamide (137 mg, 1.44 mmol), 2.5% OsO4/t-BuOH (363 μL, 1.16 mmol), NMO (226 mg, 1.93 mmol) and the mixture is stirred for 2 days. The reaction is quenched with 15% sodium bisulfate (10 mL, stirred for 5 min) then diluted with EtOAc (20 mL). The aqueous phase was separated, washed with water (10 mL) and brine (10 mL), and dried over Na₂SO₄. Evaporation of solvent gave an off-white solid (355 mg). The crude material was purified on 25 g SNAP silica cartridge, eluting with Hex/EtOAc/AcOH (30/30/1) over 25 CV, to give the title compound (206 mg (54%).

### Step III: Compound 67

To a solution of [(2R,3S,4R,5S,6R)-3-acetoxy-6-(4-chloro-3-hydroxy-phenyl)-4,5-dihydroxy-tetrahydropyran-2-yl]methyl acetate (35 mg, 0.0887 mmol) in dry MeOH (998 μL), at room temperature was added MeONa (46 μL of 25% w/v, 0.213 mmol). The reaction was stirred for 45 min, then treated with acidic resin and shaken till pH was no longer basic. The mixture was filtered and evaporated to a gum. The gum was taken into CH₃CN/water (1/1) and freeze-dried to a solid (23 mg). The gum was purified by prep HPLC to give the title compound as a white powder (13 mg, 49%).

¹H NMR (400 MHz, CD₃OD) δ 7.18 (d, 1H), 6.95 (d, 1H), 6.81 (dd, 1H), 4.24 (m, 1H), 3.79-3.70 (m, 2H), 3.64 (t, 1H), 3.43 (ddd, 2H). LC-MS: m/z=313.1 (M+Na+).

### Example 29

### Preparation of Compound 68

### (2R,3S,4R,5S,6R)-2-(2-chloro-5-hydroxyphenyl)-6-(hydroxymethyl)tetrahydro-2H-pyran-3,4,5-triol

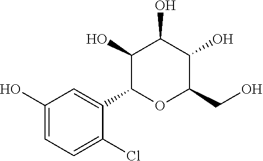 [SMILES: OC[C@H]1O[C@H](c2cc(O)ccc2Cl)[C@@H](O)[C@@H](O)[C@@H]1O]

COMPOUND 68 is prepared according to similar procedure described for COMPOUND 67 but using (2-chloro-3-hydroxy-phenyl)boronic acid.

¹H NMR (400 MHz, CD₃OD) δ 7.11 (dd, 2H), 6.68 (dd, 1H), 5.21 (d, 1H), 4.30-3.93 (m, 2H), 3.94-3.55 (m, 4H). LC-MS: m/z=290.1 (M+).

### Example 30

### Preparation of Compound 69

### 3-((2R,3S,4R,5S,6R)-3,4,5-trihydroxy-6-(hydroxymethyl)tetrahydro-2H-pyran-2-yl)phenyl thiophen-2-ylcarbamate

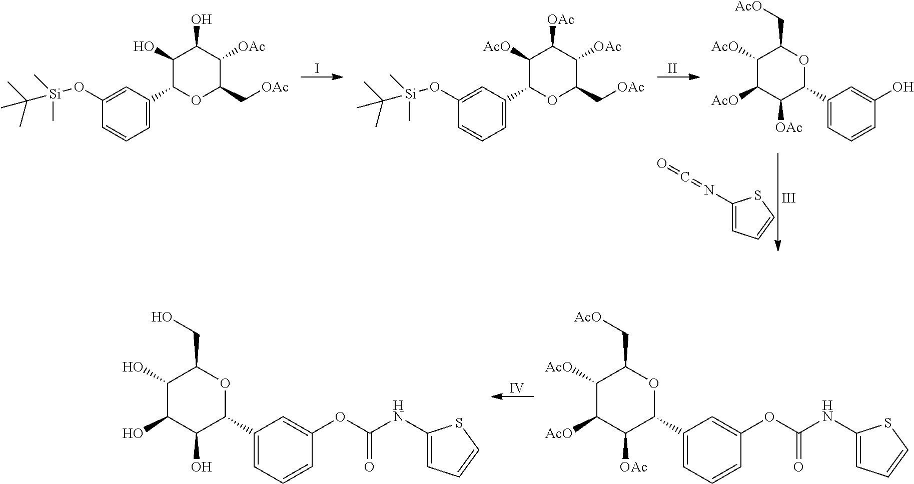

### Step I: (2R,3R,4R,5R,6R)-2-(acetoxymethyl)-6-(3-((tert-butyldimethylsilyl)oxy)phenyl)tetrahydro-2H-pyran-3,4,5-triyltriacetate

To a solution of [(2R,3S,4R,5S,6R)-3-acetoxy-6-[3-[tert-butyl(dimethyl)silyl]oxyphenyl]-4,5-dihydroxy-tetrahydropyran-2-yl]methyl acetate (INTERMEDIATE A, Step II) (1.00 g, 2.20 mmol) in CH₂Cl₂ (20 mL) at 0° C., under N₂ atmosphere was added leutidine (872 μL, 6.60 mmol) followed by DMAP (54 mg, 0.440 mmol and acetic anhydride (623 μL, 6.60 mmol). The yellow solution was stirred at 0° C. for 1.5 h. The reaction mixture was treated with KHSO₄ (15%, 2×6 ml) then washed with brine, dried and evaporated to a gum (1.10 g). The crude material was purified on SNAP column using Hex/EtOAc (0-5%; 3 CV, 5-30%; 20 CV) as the eluent to give a clear gum (1.02 g 82%).

### Step II: (2R,3R,4R,5R,6R)-2-(acetoxymethyl)-6-(3-hydroxyphenyl)tetrahydro-2H-pyran-3,4,5-triyltriacetate

To a stirred solution of (2R,3R,4R,5R,6R)-2-(acetoxymethyl)-6-(3-((tert-butyldimethylsilyl)oxy)phenyl)tetrahydro-2H-pyran-3,4,5-triyltriacetate (1.02 g, 1.89 mmol) in THF (11 mL) are added acetic acid (162 μL, 2.841 mmol) and TBAF (5.68 mL of 1 M, 5.68 mmol). The mixture was stirred at room temperature for 30 min then was diluted with EtOAc (30 mL), washed with water (20 mL) and brine (20 mL), dried over Na₂SO₄, and concentrated to give a clear oil which solidified to a wax. The crude material was purified on SNAP column (25 g) eluting with Hex/EtOAc (5%; SCV, 5-30%; 25 CV, 30-40%; SCV, 40-50%; 30 CV) to give the title compound as a white foam (409 mg, 48%)

### Step III: Compound 69

### 3-((2R,3S,4R,5S,6R)-3,4,5-trihydroxy-6-(hydroxymethyl)tetrahydro-2H-pyran-2-yl)phenyl thiophen-2-ylcarbamate

To a solution of [(2R,3R,4R,5R)-3,4,5-triacetoxy-6-(3-hydroxyphenyl)tetrahydropyran-2-yl]methyl acetate (50 mg, 0.118 mmol) in CH₂Cl₂ (500 L) was added under N₂ atmosphere molecular sieves (100 mg) followed by triethylamine (49 L, 0.35 mmol) followed by isocyanatothiophene (44 mg, 0.35 mmol). The reaction was filtered and the filtrate evaporated to dryness. The residual crude material was purified by reverse phase HPLC to give after freeze-drying 6.2 mg of the title compound (13%)

LC-MS: m/z=380.9 (M+H⁺).

### Example 31

### Preparation of Compounds 70-72

COMPOUNDS 70-72 listed in Table 3 are prepared according to similar procedure described for COMPOUND 69 but using the appropriate isocyanate.

TABLE 3

| | | LC-MS: |
|---|---|---|
| COMPOUND | Structure and Name | m/z (M + H⁺) |
| 70 | 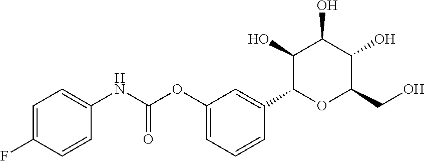 [SMILES: O=C(Nc1ccc(F)cc1)Oc1cccc([C@H]2O[C@H](CO)[C@@H](O)[C@H](O)[C@@H]2O)c1] | 393.1 |
| | 3-((2R,3S,4R,5S,6R)-3,4,5-trihydroxy-6- | |
| | (hydroxymethyl)tetrahydro-2H-pyran-2-yl)phenyl (4- | |
| | fluorophenyl)carbamate | |
| 71 | 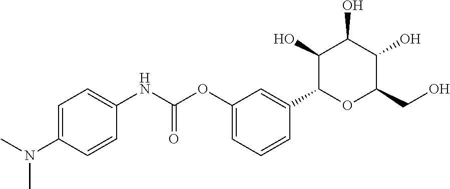 [SMILES: CN(C)c1ccc(NC(=O)Oc2cccc([C@H]3O[C@H](CO)[C@@H](O)[C@H](O)[C@@H]3O)c2)cc1] | 417.9 |
| | 3-((2R,3S,4R,5S,6R)-3,4,5-trihydroxy-6- | |
| | (hydroxymethyl)tetrahydro-2H-pyran-2-yl)phenyl (4- | |
| | (dimethylamino)phenyl)carbamate | |
| 72 | 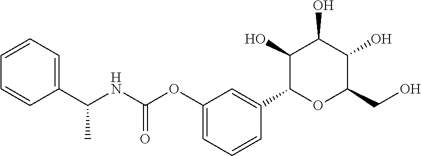 [SMILES: C[C@@H](NC(=O)Oc1cccc([C@H]2O[C@H](CO)[C@@H](O)[C@H](O)[C@@H]2O)c1)c1ccccc1] | 404.1 |
| | 3-((2R,3S,4R,5S,6R)-3,4,5-trihydroxy-6- | |
| | (hydroxymethyl)tetrahydro-2H-pyran-2-yl)phenyl (R)-1- | |
| | phenylethylcarbamate | |

### Preparation of Compound 73

### ((2R,3S,4R,5S,6R)-2-(3-Ethynylphenyl)-6-(hydroxymethyl)tetrahydropyran-3,4,5-triol)

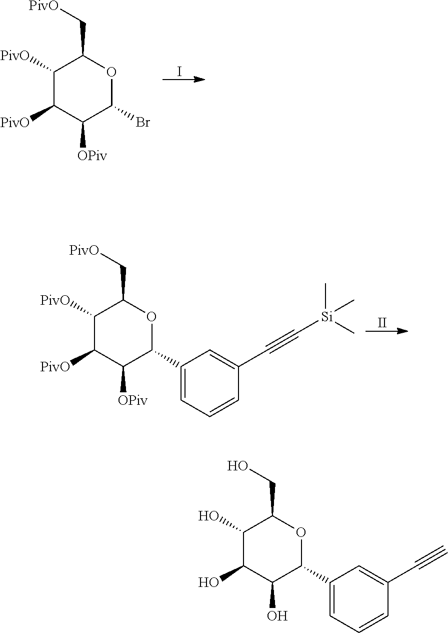

### Step I: [(2R,3R,4R,5R,6R)-3,4,5-tris(2,2-dimethylpropanoyloxy)-6-[3-(2-trimethylsilylethynyl)phenyl]tetrahydropyran-2-yl]methyl 2,2-dimethylpropanoate

A solution of n-Bu₃MgLi (2.65 mL of 0.65 M, 1.725 mmol) in hexane-heptane-dibutylether (8:20:3) is added to 2-(3-bromophenyl)ethynyl-trimethyl-silane (1.248 g, 1.05 mL, 4.928 mmol) in toluene (2.4 mL) and dibutylether (1.4 mL) at 0° C. and stirred in cold room for 25 h. A solution of ZnBr₂—LiBr in dibutyl ether (2.6 mL of 1.05 M, 2.711 mmol) is added dropwise, cooling bath removed, stirred at room temperature for 1 h. A solution of [(2R,3R,4S,5S,6R)-6-bromo-3,4,5-tris(2,2-dimethylpropanoyloxy)tetrahydropyran-2-yl]methyl 2,2-dimethylpropanoate (2.38 g, 4.107 mmol) in toluene (4.3 mL) is added, it is placed on pre-heated oil bath at 90° C., stirred over weekend. The reaction mixture is cooled to room temperature, it is poured into aq. 1 N HCl solution (40 mL) and extracted with ethyl acetate (3×40 mL). The combined extracts are washed with brine, dried (Na₂SO₄), concentrated, purified on Biotage™ 100 g SNAP silica gel cartridge using ethyl acetate in hexanes (0% to 10%, 12 CV, 10%, 5 CV) as eluent to afford the title compound (765 mg) as an oil.

### Step II. Compound 73

To a stirred light suspension of [(2R,3R,4R,5R,6R)-3,4,5-tris(2,2-dimethylpropanoyloxy)-6-[3-(2-trimethylsilylethynyl)phenyl]tetrahydropyran-2-yl]methyl 2,2-dimethylpropanoate (765 mg, 1.137 mmol) in methanol (15 mL) is added methanolate (Sodium Ion (1)) (4.6 mL of 0.5 M, 2.274 mmol) and stirred at room temperature for 24 h. To the resultant solution is added DOWEX 50WX4-400 until pH 4-5, filtered, eluted with methanol. The filtrate is concentrated, purified on Biotage™ 40 g silica gel SNAP cartridge using EtOAc-MeOH—H₂O (47.5:1.5:1 to 10:1.5:1) as eluent to afford title product (170 mg, 55%) as beige solid.

LC-MS: m/z=265.28 (M+H⁺).

### Preparation of Compound 74

### (2R,3S,4R,5S,6R)-2-(4-Ethynylphenyl)-6-(hydroxymethyl)tetrahydropyran-3,4,5-triol

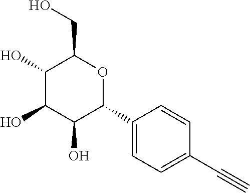 [SMILES: C#Cc1ccc([C@H]2O[C@H](CO)[C@@H](O)[C@H](O)[C@@H]2O)cc1]

The title compound is prepared from 2-(4-iodophenyl)ethynyl-trimethyl-silane as described in COMPOUND 73

¹H NMR (400 MHz, CD₃OD) δ 7.45 (s, 4H), 4.93 (d, J=4.1 Hz, 1H), 4.38-4.33 (m, 1H), 3.87-3.76 (m, 2H), 3.72 (t, J=7.7 Hz, 1H), 3.55 (dd, J=7.8, 3.1 Hz, 1H), 3.49-3.42 (m, 1H), 3.46 (s, 1H).

### Preparation of Compound 75

### (2R,3S,4R,5S,6R)-2-(3-Ethynyl-4-methoxy-phenyl)-6-(hydroxymethyl)tetrahydropyran-3,4,5-triol

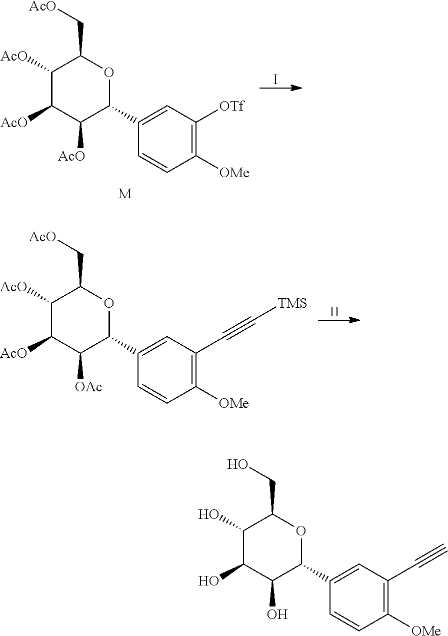

### Step I: [(2R,3R,4R,5R,6R)-3,4,5-triacetoxy-6-[4-methoxy-3-(2-trimethylsilylethynyl)phenyl]tetrahydropyran-2-yl]methyl acetate

The title compound is prepared from INTERMEDIATE M using similar procedure described for INTERMEDIATE F

### Step II: Compound 75

To a solution of [(2R,3R,4R,5R,6R)-3,4,5-triacetoxy-6-[4-methoxy-3-(2-trimethylsilylethynyl)phenyl]tetrahydropyran-2-yl]methyl acetate (145 mg, 0.2712 mmol) in methanol (2 mL) is added a solution of sodium methoxide in methanol (60 μL of 25% w/v, 0.278 mmol), reaction mixture is stirred at room temperature for 18 hours, quenched with ion-exchange acid resin (DOWEX 50WX4-400) until pH 5-6, filtered, washed with dry methanol, combined organic solution is concentrated. Purified on reverse phase 25 g C18 silica gel column on Isolera system using a gradient of acetonitrile in water (5%, 3 CV; 5% to 15%, 8 CV; 15% 2 CV) as eluent to afford title compound (66 mg, 0.2168 mmol, 80%) as white solid.

¹H NMR (400 MHz, CD₃OD) δ 7.50 (d, J=2.1 Hz, 1H), 7.44 (dd, J=8.9, 2.0 Hz, 1H), 6.99 (d, J=8.7 Hz, 1H), 4.86-4.84 (m, 1H), 4.34-4.28 (m, 1H), 3.84 (s, 3H), 3.83-3.75 (m, 2H), 3.72 (t, J=7.6 Hz, 1H), 3.60 (dd, J=7.7, 3.1 Hz, 1H), 3.57 (s, 1H), 3.48-3.40 (m, 1H). LC-MS: m/z=295.32 (M+H⁺).

### Preparation of Compound 76

### (2R,3S,4R,5S,6R)-2-(3-Bromo-2-methyl-phenyl)-6-(hydroxymethyl)tetrahydropyran-3,4,5-triol

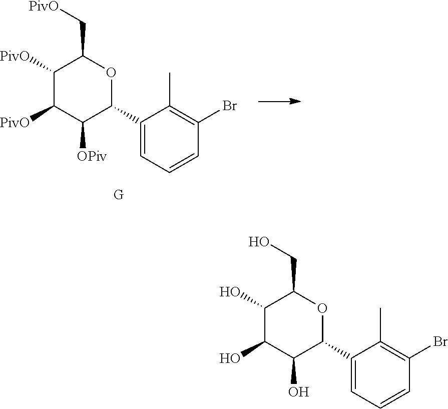

To a stirred suspension of INTERMEDIATE G (390 mg, 0.5824 mmol) in methanol (7.2 mL) is added a solution of sodium methoxide in methanol (3.5 mL of 0.5 M, 1.75 mmol), stirred at room temperature for 24 hours, to the resultant solution is added DOWEX 50WX4-400 until pH 4-5, filtered, eluted with methanol, filtrate is concentrated. The residue is purified on 60 g C18 silica gel cartridge on Isolera system using a gradient of acetonitrile in water (10%, 2 CV; 10% to 45%, 7 CV; 45%, 3 CV) as eluent to afford title compound (60 mg, 30.1%) as white solid.

¹H NMR (400 MHz, CD₃OD) δ 7.48 (t, J=7.5 Hz, 2H), 7.07 (t, J=7.9 Hz, 1H), 5.13 (d, J=7.1 Hz, 1H), 4.18-4.11 (m, 1H), 4.02 (dd, J=11.9, 7.4 Hz, 1H), 3.98-3.91 (m, 1H), 3.82 (dd, J=5.4, 4.2 Hz, 1H), 3.71 (dd, J=12.0, 3.8 Hz, 1H), 3.63-3.55 (m, 1H), 2.51 (s, 3H). LC-MS: m/z=333.29 (M+H⁺).

### Preparation of Compound 77

### 2R,3S,4R,5S,6R)-2-(4-Bromo-3-methoxy-phenyl)-6-(hydroxymethyl)tetrahydropyran-3,4,5-triol

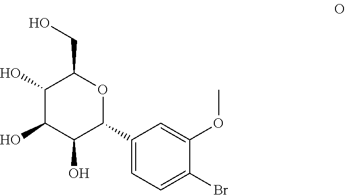 [SMILES: COc1cc([C@H]2O[C@H](CO)[C@@H](O)[C@H](O)[C@@H]2O)ccc1Br]

The title compound is prepared starting from 4-iodo-2-methoxy-benzene as described in INTERMEDIATE C.

¹H NMR (400 MHz, CDC30D) δ 7.48 (d, J=8.2 Hz, 1H), 7.21 (s, 1H), 6.90 (d, J=8.0 Hz, 1H), 4.89 (d, J=4.0 Hz, 1H), 4.33 (t, J=3.5 Hz, 1H), 3.87 (s, 3H), 3.82 (d, J=4.8 Hz, 2H), 3.69 (t, J=7.6 Hz, 1H), 3.57 (dd, J=7.7, 3.0 Hz, 1H), 3.53-3.45 (m, 1H).

### Preparation of Compound 78

### (2R,3S,4R,5S,6R)-2-(3-Chloro-2-fluoro-phenyl)-6-(hydroxymethyl)tetrahydropyran-3,4,5-triol

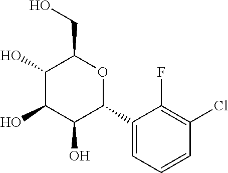 [SMILES: OC[C@H]1O[C@H](c2cccc(Cl)c2F)[C@@H](O)[C@@H](O)[C@@H]1O]

The title product is prepared from the 1-chloro-2-fluoro-3-iodo-benzene as described in COMPOUND 77

¹H NMR (400 MHz, CD₃OD) δ 7.60-7.52 (m, 1H), 7.42-7.35 (m, 1H), 7.16 (t, J=7.9 Hz, 1H), 5.14 (d, J=7.4 Hz, 1H), 4.14-4.00 (m, 2H), 3.88-3.81 (m, 2H), 3.80-3.69 (m, 2H). LC-MS: m/z=293.14 (M+H⁺).

### Preparation of Compound 79

### (2R,3S,4R,5S,6R)-2-[3-(3,5-Dichlorophenyl)phenyl]-6-(hydroxymethyl)tetrahydropyran-3,4,5-triol

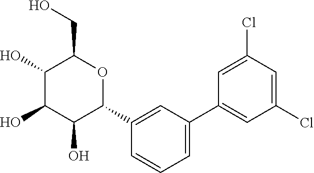 [SMILES: OC[C@H]1O[C@H](c2cccc(-c3cc(Cl)cc(Cl)c3)c2)[C@@H](O)[C@@H](O)[C@@H]1O]

To a solution of INTERMEDIATE C (40 mg, 0.125 mmol) and (3,5-dichlorophenyl)boronic acid (48 mg, 0.2515 mmol) in MeOH (2.5 mL) in microwave vial (10 mL) is added K₂CO₃ (35 mg, 0.253 mmol) and SiliaCat DPP-Pd (144.6 mg, 0.0376 mmol) and heated in microwave for 20 min at 100° C., diluted with methanol-CH₂Cl₂-Water, filtered off SilicaCat, and the filtrate was concentrated. The residue is dissolved in methanol-water, neutralized with DOWEX 500 until pH 5, filtered off. The filtrate is concentrated, purified on reverse phase HPLC to afford the tile compound (7 mg, 14.5%) as white solid.

HPLC details: Phenomenex C18 Gemini AXIA 5μ 110 Å 21.2×250 mm; using acetonitrile in water (10% to 60%, 40 min, with 0.01% TFA as buffer).

¹H NMR (400 MHz, CD₃OD) δ 7.79 (brs, 1H), 7.63 (d, J=1.9 Hz, 2H), 7.58-7.46 (m, 3H), 7.42 (t, J=1.9 Hz, 1H), 5.01 (d, J=4.4 Hz, 1H), 4.43 (dd, J=4.4, 3.2 Hz, 1H), 3.93-3.80 (m, 2H), 3.76 (t, J=7.3 Hz, 1H), 3.66 (dd, J=7.5, 3.1 Hz, 1H), 3.56 (td, J=7.1, 3.1 Hz, 1H). LC-MS: m/z=385.2 (M+H⁺).

### Preparation of Compound 80

### Methyl 3-[[4-[4-[(2R,3S,4R,5S,6R)-3,4,5-trihydroxy-6-(hydroxymethyl)tetrahydropyran-2-yl]phenyl]triazol-1-yl]methyl]benzoate

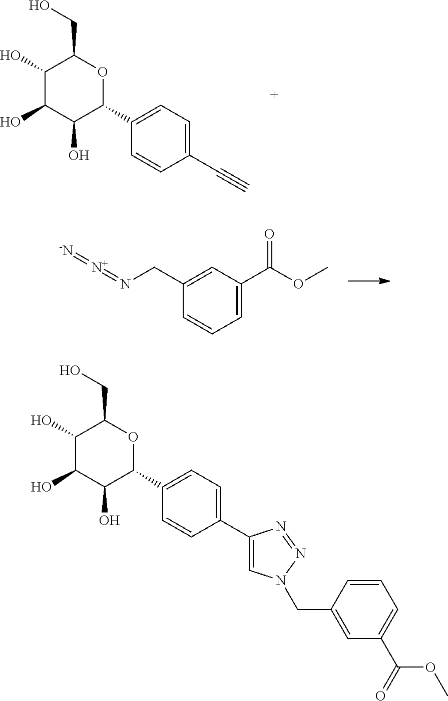

To a stirred solution of methyl 3-(azidomethyl)benzoate (26.0 mg, 0.1362 mmol) and COMPOUND 74 (30 mg, 0.114 mmol) in EtOH (520 μL) and H₂O (173 μL) is added CuSO₄ (9.1 mg, 0.057 mmol). To the resulting light blue colored suspension is added (2R)-2-[(1S)-1,2-dihydroxyethyl]-4-hydroxy-5-oxo-2H-furan-3-olate (562.2 mg, 2.84 mmol) (Sodium ascorbate). The reaction flask is sealed, the resultant suspension is stirred at room temperature over the weekend, diluted with water, washed with methylene chloride. The resulting aqueous suspension is filtered through 0.4 micron filter, concentrated, and the resulting solid purified on 25 g C18 SNAP silica gel cartridge using a gradient of acetonitrile in water (5% to 35%) as eluent to afford title compound (15 mg, 27.6%) as white solid.

¹H NMR (400 MHz, CD₃OD) δ 8.36 (s, 1H), 8.01 (s, 1H), 7.98 (d, J=7.8 Hz, 1H), 7.80 (d, J=8.3 Hz, 2H), 7.60 (d, J=7.7 Hz, 1H), 7.56-7.46 (m, 3H), 5.69 (s, 2H), 4.97 (d, J=3.5 Hz, 1H), 4.42 (t, J=3.4 Hz, 1H), 3.87 (s, 3H), 3.84-3.81 (m, 2H), 3.74 (t, J=7.9 Hz, 1H), 3.59 (dd, J=8.0, 3.0 Hz, 1H), 3.52-3.45 (m, 1H). LC-MS: m/z=456.43 (M+H⁺).

### Preparation of Compound 81

### (2R,3S,4R,5S,6R)-2-[4-(1-Benzyltriazol-4-yl)phenyl]-6-(hydroxymethyl)tetrahydropyran-3,4,5-triol

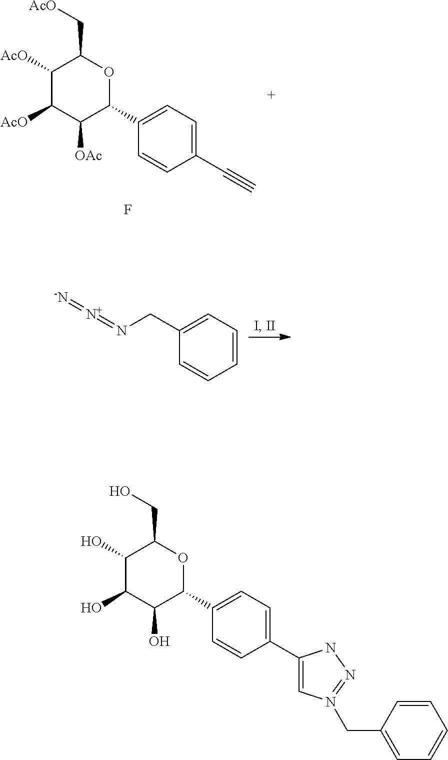

The title compound is prepared from INTERMEDIATE F and azidomethylbenzene as described for COMPOUND 80, followed by a standard deprotection of the acetates using NaOMe/MeOH.

¹H NMR (400 MHz, CD₃OD) δ 8.31 (s, 1H), 7.80 (d, J=8.4 Hz, 2H), 7.53 (d, J=8.1 Hz, 2H), 7.41-7.29 (m, 5H), 5.62 (s, 2H), 4.97 (d, J=3.7 Hz, 1H), 4.42 (t, J=3.5 Hz, 1H), 3.85-3.79 (m, 2H), 3.73 (t, J=7.9 Hz, 1H), 3.58 (dd, J=8.0, 3.1 Hz, 1H), 3.52-3.44 (m, 1H). LC-MS: m/z=398.53 (M+H⁺).

### Preparation of Compound 82

### Dimethyl 5-[2-[2-methoxy-5-[(2R,3S,4R,5S,6R)-3,4,5-trihydroxy-6-(hydroxymethyl)tetrahydropyran-2-yl]phenyl]ethynyl]benzene-1,3-dicarboxylate

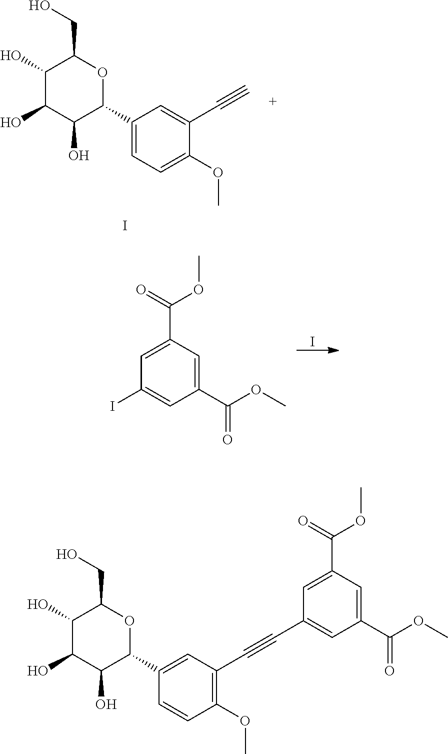

To a degassed (purged with nitrogen gas for 5 min) mixture of dimethyl 5-iodobenzene-1,3-dicarboxylate (18.9 mg, 0.059 mmol) and CuI (1.9 mg, 0.01 mmol) and Pd(dppf)Cl₂—CH₂Cl₂ (4.0 mg, 0.005 mmol) in DMF (2 mL) is added sequentially, TEA (29.92 mg, 41.0 μL, 0.296 mmol) and COMPOUND 75 (15 mg, 0.0493 mmol), dark brown reaction mixture is heated at 50° C. for 18 hours, filtered through 0.4 micron filter, purified on prep. HPLC (Injected on Phenomenex C18 Gemini AXIA Sum 110 A 21.2×75 mm Hold 10 min-10% ACN/H₂O+0.1% Formic Acid-To 60% ACN+0.1% Formic Acid in 40 min) to afford title compound (13 mg, 51.3%) a s white solid.

¹H NMR (400 MHz, CD₃OD) δ 8.47 (t, J=1.6 Hz, 1H), 8.21 (d, J=1.6 Hz, 2H), 7.53 (d, J=2.0 Hz, 1H), 7.42 (dd, J=8.7, 2.3 Hz, 1H), 6.98 (d, J=8.7 Hz, 1H), 4.84-4.80 (m, 1H), 4.30-4.25 (m, 1H), 3.87 (s, 6H), 3.84 (s, 3H), 3.81-3.69 (m, 2H), 3.65 (t, J=7.5 Hz, 1H), 3.56 (dd, J=7.7, 3.1 Hz, 1H), 3.44-3.36 (m, 1H). LC-MS: m/z=487.47 (M+H⁺).

### Preparation of Compound 83

### 3-[2-[2-Methoxy-5-[(2R,3S,4R,5S,6R)-3,4,5-trihydroxy-6-(hydroxymethyl)tetrahydropyran-2-yl]phenyl]ethynyl]-N-methyl-benzamide

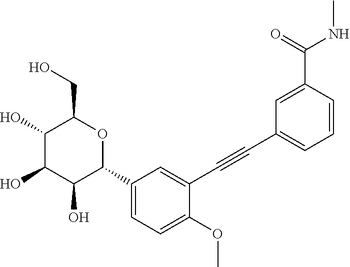 [SMILES: CNC(=O)c1cccc(C#Cc2cc([C@H]3O[C@H](CO)[C@@H](O)[C@H](O)[C@@H]3O)ccc2OC)c1]

The title compound is prepared as described for COMPOUND 82 using commercially available 3-iodo-N-methylbenzamide.

LC-MS: m/z=428.43 (M+H⁺).

### Preparation of Compound 84

### 5-[2-[2-Methoxy-5-[(2R,3S,4R,5S,6R)-3,4,5-trihydroxy-6-(hydroxymethyl)tetrahydropyran-2-yl]phenyl]ethynyl]-N1,N3-dimethyl-benzene-1,3-dicarboxamide

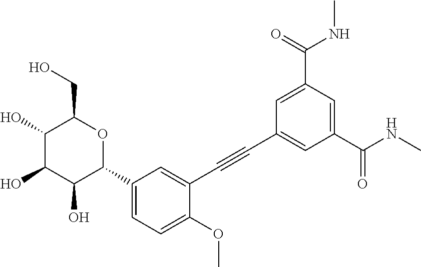 [SMILES: CNC(=O)c1cc(C#Cc2cc([C@H]3O[C@H](CO)[C@@H](O)[C@H](O)[C@@H]3O)ccc2OC)cc(C(=O)NC)c1]

The title compound is prepared as described for COMPOUND 82 using commercially available 5-iodo-N1,N3-dimethylisophthalamide.

¹H NMR (400 MHz, CD₃OD) δ 8.22 (t, J=1.6 Hz, 1H), 8.06 (d, J=1.6 Hz, 2H), 7.58 (d, J=2.0 Hz, 1H), 7.51-7.45 (m, 1H), 7.05 (d, J=8.7 Hz, 1H), 4.90-4.86 (m, 1H), 4.38-4.32 (m, 1H), 3.90 (s, 3H), 3.89-3.77 (m, 2H), 3.74 (t, J=7.5 Hz, 1H), 3.63 (dd, J=7.7, 3.1 Hz, 1H), 3.52-3.43 (m, 1H), 2.92 (s, 6H). LC-MS: m/z=485.48 (M+H⁺).

### Preparation of Compound 85

### N1,N3-Dimethyl-5-[2-[3-[(2R,3S,4R,5S,6R)-3,4,5-trihydroxy-6-(hydroxymethyl)tetrahydropyran-2-yl]phenyl]ethynyl]benzene-1,3-dicarboxamide

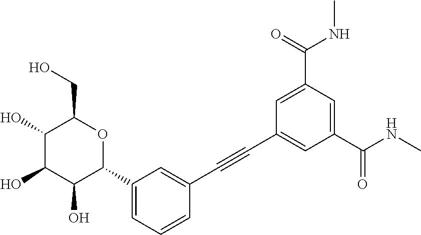 [SMILES: CNC(=O)c1cc(C#Cc2cccc([C@H]3O[C@H](CO)[C@@H](O)[C@H](O)[C@@H]3O)c2)cc(C(=O)NC)c1]

The title compound is prepared as described for COMPOUND 73 using commercially available 5-iodo-N1,N3-dimethylisophthalamide.

¹H NMR (400 MHz, CD₃OD) δ 8.15 (t, J=1.6 Hz, 1H), 8.00 (d, J=1.7 Hz, 2H), 7.60 (s, 1H), 7.45 (d, J=7.7 Hz, 1H), 7.39 (d, J=7.6 Hz, 1H), 7.33 (t, J=7.6 Hz, 1H), 4.86 (d, J=4.3 Hz, 1H), 4.32-4.25 (m, 1H), 3.84-3.64 (m, 3H), 3.53 (dd, J=7.5, 3.1 Hz, 1H), 3.48-3.40 (m, 1H), 2.84 (s, 6H). LC-MS: m/z=455.41 (M+H⁺).

### Preparation of Compound 86

### Methyl 3-[2-[3-[(2R,3S,4R,5S,6R)-3,4,5-trihydroxy-6-(hydroxymethyl)tetrahydropyran-2-yl]phenyl]ethynyl]benzoate

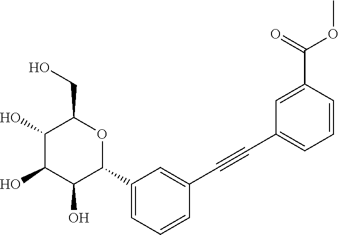 [SMILES: COC(=O)c1cccc(C#Cc2cccc([C@H]3O[C@H](CO)[C@@H](O)[C@H](O)[C@@H]3O)c2)c1]

The title compound is prepared as described for COMPOUND 82 using COMPOUND 73 and commercially available methyl 3-iodobenzoate.

¹H NMR (400 MHz, CD₃OD) δ 8.12 (t, J=1.5 Hz, 1H), 8.02-7.96 (m, 1H), 7.76-7.71 (m, 1H), 7.69-7.66 (m, 1H), 7.55-7.36 (m, 4H), 4.94 (d, J=4.3 Hz, 1H), 4.41-4.34 (m, 1H), 3.91 (s, 3H), 3.89-3.79 (m, 2H), 3.75 (t, J=7.5 Hz, 1H), 3.62 (dd, J=7.6, 3.1 Hz, 1H), 3.56-3.47 (m, 1H). LC-MS: m/z=399.88 (M+H⁺).

### Preparation of Compound 87

### Methyl 4-[2-[3-[(2R,3S,4R,5S,6R)-3,4,5-trihydroxy-6-(hydroxymethyl)tetrahydropyran-2-yl]phenyl]ethynyl]benzoate

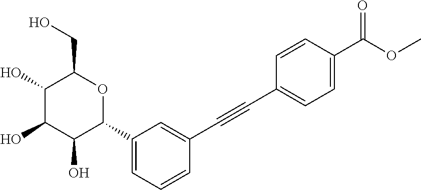 [SMILES: COC(=O)c1ccc(C#Cc2cccc([C@H]3O[C@H](CO)[C@@H](O)[C@H](O)[C@@H]3O)c2)cc1]

The title compound is prepared as described for COMPOUND 82 using COMPOUND 73 commercially available methyl 4-iodobenzoate.

¹H NMR (400 MHz, CD₃OD) δ 8.06-7.97 (m, 2H), 7.68 (s, 1H), 7.63-7.58 (m, 2H), 7.53 (d, J=8.1 Hz, 1H), 7.46 (d, J=7.6 Hz, 1H), 7.40 (t, J=7.7 Hz, 1H), 4.94 (d, J=4.3 Hz, 1H), 4.40-4.33 (m, 1H), 3.90 (s, 3H), 3.88-3.78 (m, 2H), 3.75 (t, J=7.4 Hz, 1H), 3.61 (dd, J=7.6, 3.1 Hz, 1H), 3.55-3.47 (m, 1H). LC-MS: m/z=399.38 (M+H⁺).

### Preparation of Compound 88

### Methyl 3-[2-[4-[(2R,3S,4R,5S,6R)-3,4,5-trihydroxy-6-(hydroxymethyl)tetrahydropyran-2-yl]phenyl]ethynyl]benzoate

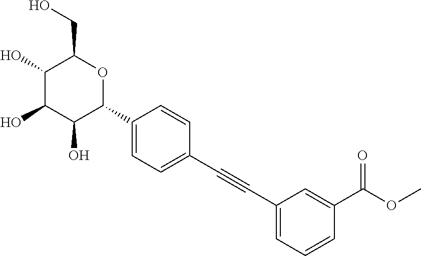 [SMILES: COC(=O)c1cccc(C#Cc2ccc([C@H]3O[C@H](CO)[C@@H](O)[C@H](O)[C@@H]3O)cc2)c1]

The title compound is prepared as described for COMPOUND 82 using COMPOUND 74 and commercially available methyl 3-iodobenzoate.

¹H NMR (400 MHz, CD₃OD) δ 8.11 (t, J=1.5 Hz, 1H), 8.01-7.95 (m, 1H), 7.76-7.70 (m, 1H), 7.60-7.43 (m, 3H), 4.96 (d, J=4.0 Hz, 1H), 4.42-4.33 (m, 1H), 3.91 (s, 3H), 3.88-3.79 (m, 1H), 3.74 (t, J=7.6 Hz, 1H), 3.58 (dd, J=7.8, 3.1 Hz, 1H), 3.52-3.44 (m, 1H). LC-MS: m/z=399.34 (M+H⁺).

### Preparation of Compound 89

### Methyl 4-[2-[4-[(2R,3S,4R,5S,6R)-3,4,5-trihydroxy-6-(hydroxymethyl)tetrahydropyran-2-yl]phenyl]ethynyl]benzoate

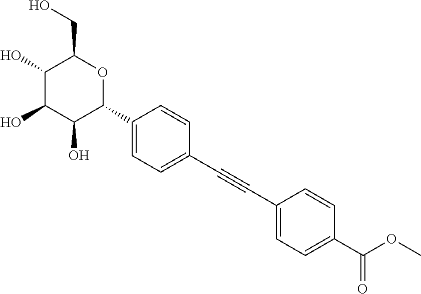 [SMILES: COC(=O)c1ccc(C#Cc2ccc([C@H]3O[C@H](CO)[C@@H](O)[C@H](O)[C@@H]3O)cc2)cc1]

The title compound is prepared as described for COMPOUND 82 using COMPOUND 74 and commercially available methyl 4-iodobenzoate.

¹H NMR (400 MHz, CD₃OD) δ 8.01 (d, J=8.5 Hz, 2H), 7.60 (d, J=8.5 Hz, 2H), 7.57-7.49 (m, 4H), 4.96 (d, J=4.1 Hz, 1H), 4.40-4.33 (m, 1H), 3.90 (s, 3H), 3.87-3.78 (m, 2H), 3.74 (t, J=7.6 Hz, 1H), 3.58 (dd, J=7.7, 3.1 Hz, 1H), 3.53-3.45 (m, 1H). LC-MS: m/z=399.38 (M+H⁺).

### Preparation of Compound 90

### N1,N3-Dimethyl-5-[2-[2-methyl-3-[(2R,3S,4R,5S,6R)-3,4,5-trihydroxy-6-(hydroxymethyl)tetrahydropyran-2-yl]phenyl]ethynyl]benzene-1,3-dicarboxamide

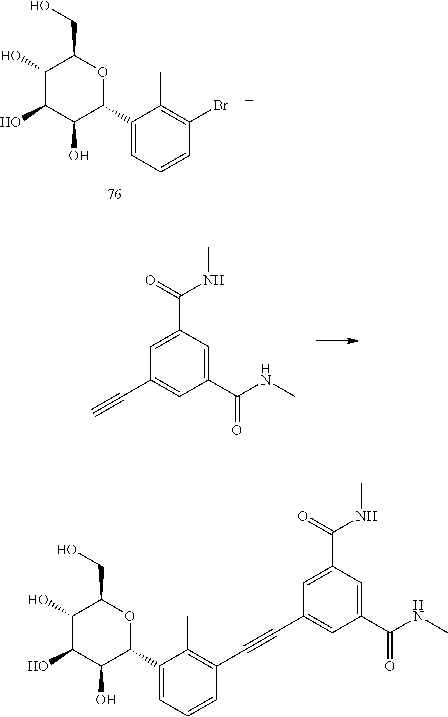 [SMILES: Cc1c(Br)cccc1[C@H]1O[C@H](CO)[C@@H](O)[C@H](O)[C@@H]1O ; C#Cc1cc(C(=O)NC)cc(C(=O)NC)c1 ; CNC(=O)c1cc(C#Cc2cccc([C@H]3O[C@H](CO)[C@@H](O)[C@H](O)[C@@H]3O)c2C)cc(C(=O)NC)c1]

The title compound is prepared using COMPOUND 76 and 5-ethynyl-N1,N3-dimethylisophthalamide according to the procedure described for COMPOUND 82.

¹H NMR (400 MHz, CD₃OD) δ 7.53-7.43 (m, 2H), 7.08 (t, J=7.9 Hz, 1H), 5.13 (d, J=7.2 Hz, 1H), 4.14 (dd, J=7.1, 3.2 Hz, 1H), 4.02 (dd, J=11.9, 7.5 Hz, 1H), 3.95 (dd, J=5.4, 3.3 Hz, 1H), 3.82 (dd, J=5.4, 4.1 Hz, 1H), 3.71 (dd, J=12.0, 3.8 Hz, 1H), 3.64-3.56 (m, 1H), 2.64 (s, 6H), 2.51 (s, 3H). LC-MS: m/z=469.51 (M+H⁺).

### Preparation of N1,N3-dimethyl-5-(2-trimethylsilylethynyl)benzene-1,3-dicarboxamide (XX) and 5-ethynyl-N1,N3-dimethyl-benzene-1,3-dicarboxamide (YY)

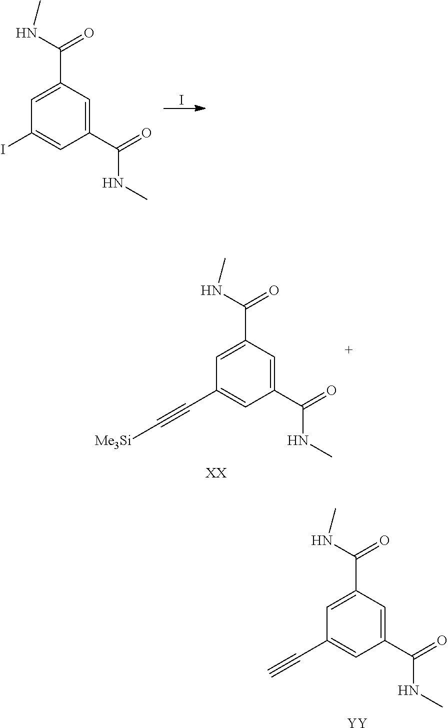

To a degassed (purged with nitrogen gas for 5 min) 5-iodo-N1,N3-dimethyl-benzene-1,3-dicarboxamide (600 mg, 1.886 mmol) and CuI (71.8 mg, 0.377 mmol) in DMF (6.0 mL) is added sequentially Pd(dppf)Cl₂-CH₂Cl₂ (154.0 mg, 0.1886 mmol), TEA (954 mg, 1.3 mL, 9.43 mmol) and ethynyl(trimethyl)silane (926 mg, 1.33 mL, 9.43 mmol), dark brown reaction mixture is heated at 50° C. for 3 hours, diluted with water (5 mL). Reaction mixture is extracted with ethyl acetate (3×10 mL), combined extracts are washed with brine, concentrated, purified on 50 g SNAP silica gel cartridge using methanol in dichloromethane (2%, 4 CV; 2% to 4%, 8 CV; 4%, 2 CV) as eluent to afford N1,N3-dimethyl-5-(2-trimethylsilylethynyl)benzene-1,3-dicarboxamide, XX (250 mg, 46%) and 5-ethynyl-N1,N3-dimethyl-benzene-1,3-dicarboxamide, YY, (80 mg, 0.2474 mmol, 13.12%).

¹H NMR (400 MHz, CD₃OD) δ 8.24 (t, J=1.7 Hz, 1H), 8.00 (d, J=1.7 Hz, 2H), 2.92 (s, 6H), 0.25 (s, 9H) and ¹H NMR (400 MHz, CD₃OD) δ 8.22 (t, J=1.6 Hz, 1H), 8.00 (d, J=1.6 Hz, 2H), 3.68 (s, 1H), 2.91 (d, J=3.6 Hz, 7H). LC-MS: m/z=217.43 (M+H⁺).

### Preparation of Compound 91

### (2R,3S,4R,5S,6R)-2-(Hydroxymethyl)-6-[3-(2-phenylethynyl)phenyl]tetrahydropyran-3,4,5-triol

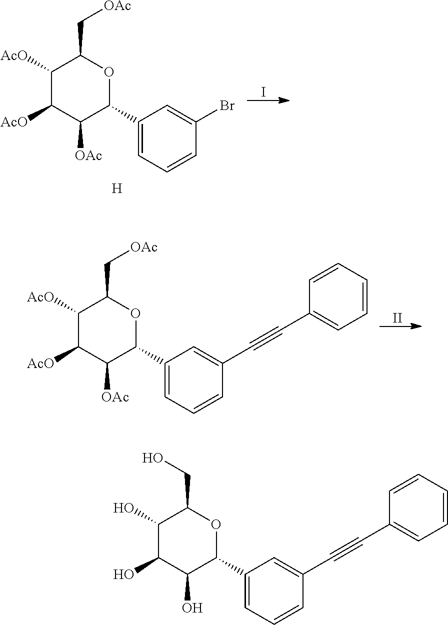

### Step I: [(2R,3R,4R,5R,6R)-3,4,5-triacetoxy-6-[3-(2-phenylethynyl)phenyl]tetrahydropyran-2-yl]methyl acetate

To a degassed (house vacuum/nitrogen flush) mixture of INTERMEDIATE H (55 mg, 0.1129 mmol), Copper Iodide (4.3 mg, 0.0226 mmol) in DMF (2.2 mL) is added PdCl₂(dppf)-CH₂Cl₂ (18.4 mg, 0.0226 mmol), Et₃N (57.12 mg, 78.7 μL, 0.565 mmol) and ethynylbenzene (34.6 mg, 37.0 μL, 0.338 mmol), heated at 80° C. for 6 hours, cooled to room temperature, diluted with water, extracted with ethyl acetate, combined extracts are washed with brine, dried (Na₂SO₄), concentrated, purified on 25 g SNAP silica gel cartridge using ethyl acetate in hexanes (15% to 50%) as eluent to afford the title compound (25 mg, 43.6%) as yellow oil.

### Step II: Compound 91

To a stirred solution of the acetates from step I in MeOH (1 mL) is added a solution of NaOMe (500 μL of 0.5 M, 0.2500 mmol, MeOH), stirred at room temperature overnight, quenched with acetic acid, concentrated, and purified by reverse phase HPLC to afford the tilte compound (23.6 mg, 61.4%) as white solid.

¹H NMR (400 MHz, CD₃OD) δ 7.56 (s, 1H), 7.45-7.23 (m, 8H), 4.86 (d, J=4.2 Hz, 1H), 4.35-4.25 (m, 1H), 3.82-3.70 (m, 2H), 3.67 (t, J=7.5 Hz, 1H), 3.53 (dd, J=7.7, 3.1 Hz, 1H), 3.46-3.36 (m, 1H). LC-MS: m/z=341.31 (M+H⁺).

### Preparation of Compound 92

### (2R,3S,4R,5S,6R)-2-[3-[2-(3,5-Dichlorophenyl)ethynyl]phenyl]-6-(hydroxymethyl)tetrahydropyran-3,4,5-triol

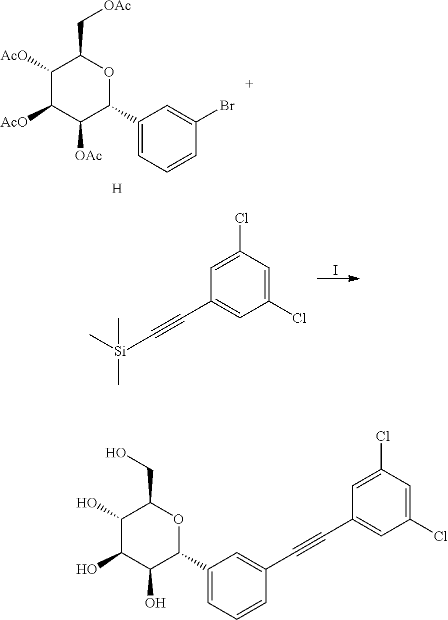

To a mixture of 2-(3,5-dichlorophenyl)ethynyl-trimethyl-silane (29.4 mg, 0.121 mmol), INTERMEDIATE H (49 mg, 0.1006 mmol), PdCl₂(dppf)-CH₂Cl₂ (16.4 mg, 0.020 mmol) and Copper Iodide (3.8 mg, 0.020 mmol) under nitrogen atmosphere is added DMF (2.0 mL), degassed twice (vacuum and nitrogen gas), DBU (119.0 μL, 0.798 mmol) and water (10.0 μL) is added, heated at 95° C. for 5 hrs, cooled to 0° C., concentrated, dissolved in DMSO (1 mL), loaded onto 3 g C18 silica gel samplet and purified on 25 g C18 on Isolera system using acetonitrile in water (10% to 50%) as eluent afforded (2R,3S,4R,5S,6R)-2-[3-[2-(3,5-dichlorophenyl)ethynyl]phenyl]-6-(hydroxymethyl)tetrahydropyran-3,4,5-triol (3 mg, 7%) as white solid.

LC-MS: m/z=409.35 (M+H⁺).

### Preparation of Compound 93

### N-Methyl-3-[2-[3-[(2R,3S,4R,5S,6R)-3,4,5-trihydroxy-6-(hydroxymethyl)tetrahydropyran-2-yl]phenyl]ethynyl]benzamide

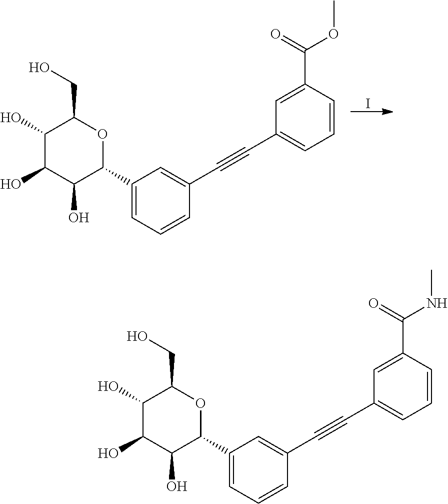

A solution of COMPOUND 86 (10 mg, 0.024 mmol) and methylamine solution in ethanol (1 mL of 33% w/w) is stirred at room temperature for 5 days. Reaction mixture is concentrated and purified on Isolera system using 12 g C18 silica gel cartridge using acetonitrile-water (10% to 50%) as eluent to afford title compound (7.5 mg, 76.6%) as solid.

¹H NMR (400 MHz, CD₃OD) δ 7.87 (t, J=1.5 Hz, 1H), 7.74-7.68 (m, 1H), 7.61-7.55 (m, 2H), 7.47-7.28 (m, 4H), 4.86 (d, J=4.3 Hz, 1H), 4.32-4.26 (m, 1H), 3.84-3.71 (m, 2H), 3.67 (t, J=7.4 Hz, 1H), 3.53 (dd, J=7.6, 3.1 Hz, 1H), 3.47-3.41 (m, 1H), 2.83 (s, 3H). LC-MS: m/z=398.4 (M+H⁺).

### Preparation of Compound 94

### N1,N3-Dimethyl-5-[(E)-3-[3-[(2R,3S,4R,5S,6R)-3,4,5-trihydroxy-6-(hydroxymethyl)tetrahydropyran-2-yl]phenyl]prop-1-enyl]benzene-1,3-dicarboxamide

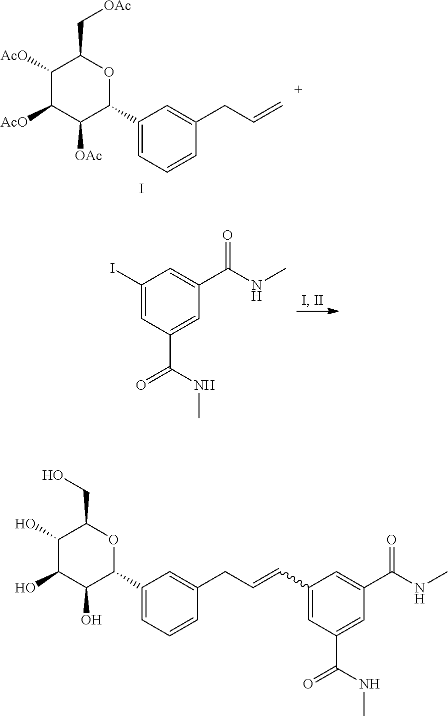

### Step I: [(2R,3R,4R,5R,6R)-3,4,5-triacetoxy-6-[3-[(E)-3-[3,5-bis(methylcarbamoyl)phenyl]allyl]phenyl]tetrahydropyran-2-yl]methyl acetate

To a solution of INTERMEDIATE I (75 mg, 0.1672 mmol) in DMF (1.9 mL) are added 5-iodo-N1,N3-dimethyl-benzene-1,3-dicarboxamide (66.5 mg, 0.209 mmol) (see preparation below), palladium acetate (6 mg, 0.0267 mmol), tetrabutylammonium bromide (53.9 mg, 0.1672 mmol) and sodium bicarbonate (42.1 mg, 0.5016 mmol). The reaction mixture is heated at 85° C. overnight under nitrogen atmosphere. Reaction mixture is quenched with water and, extracted with EtOAc (3×10 mL), combined extracts are washed with brine, dried (Na₂SO₄), concentrated, purified on 25 g SNAP silica gel cartridge on SP1 using methanol in methylene chloride (2%, 4 CV; 2% to 4%, 8 CV; 4%, 2 CV) as eluent to afford the title compound (60 mg, 56.2%) as colorless gum.

### Step II: Compound 94

To a stirred solution of [(2R,3R,4R,5R,6R)-3,4,5-triacetoxy-6-[3-[(E)-3-[3,5-bis(methylcarbamoyl)phenyl]allyl]phenyl]tetrahydropyran-2-yl]methyl acetate from step I (60 mg) in methanol (0.5 mL) is added a solution of NaOMe in methanol (500 μL of 0.5 M, 0.25 mmol), stirred at room temperature for 20 hours, quenched with DOWEX 50WX4-400 until pH 4-5, filtered, Purified on prep HPLC to afford title compound (27 mg, 34.0%) as cis-trans mixture.

LC-MS: m/z=471.38 (M+H⁺).

### Preparation of 5-iodo-N1,N3-dimethyl-benzene-1,3-dicarboxamide

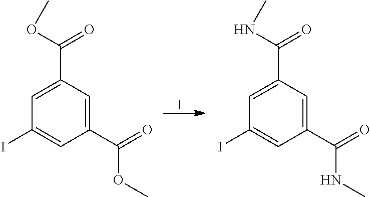

A solution of dimethyl 5-iodobenzene-1,3-dicarboxylate (2000 mg, 6.25 mmol) and methyl amine solution in ethanol (40 mL of 33% w/w) is stirred at room temperature for 5 days in a sealed tube, stopper is removed, let it stand at room temperature, product is crystallized out, filtered off through Buchner funnel to afford title compound (1.3 g, 65.4%) as light brown solid.

¹H NMR (400 MHz, CD₃OD) δ 8.27 (d, J=1.6 Hz, 2H), 8.22 (t, J=1.6 Hz, 1H), 2.90 (s, 6H).

### Preparation of Compound 95

### (2R,3S,4R,5S,6R)-2-[3-[(E)-3-(3,5-Dichlorophenyl)allyl]phenyl]-6-(hydroxymethyl)tetrahydropyran-3,4,5-triol

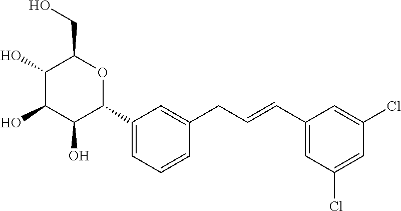 [SMILES: OC[C@H]1O[C@H](c2cccc(C/C=C/c3cc(Cl)cc(Cl)c3)c2)[C@@H](O)[C@@H](O)[C@@H]1O]

The title compound is prepared as described for COMPOUND 94 using commercially available 1,3-dichloro-5-iodobenzene.

¹H NMR (400 MHz, CD₃OD) δ 7.45 (s, 1H), 7.31-7.05 (m, 6H), 6.43 (d, J=15.8 Hz, 1H), 6.35-6.23 (m, 1H), 4.86 (t, J=4.8 Hz, 1H), 4.35-4.29 (m, 1H), 3.76-3.70 (m, 2H), 3.66-3.60 (m, 1H), 3.53-3.36 (m, 4H). LC-MS: m/z=425.23 (M+H⁺).

### Preparation of Compound 96

### (2R,3S,4R,5S,6R)-2-[3-[(E)-Cinnamyl]phenyl]-6-(hydroxymethyl)tetrahydropyran-3,4,5-triol

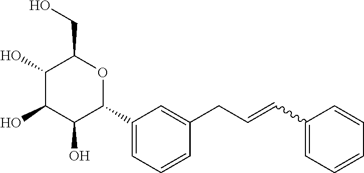 [SMILES: OC[C@H]1O[C@H](c2cccc(CC=Cc3ccccc3)c2)[C@@H](O)[C@@H](O)[C@@H]1O]

The title compound is prepared as described for COMPOUND 94 using commercially available iodobenzene.

LC-MS: m/z=357.34 (M+H⁺).

### Preparation of Compound 97

### N1,N3-Dimethyl-5-[3-[3-[(2R,3S,4R,5S,6R)-3,4,5-trihydroxy-6-(hydroxymethyl)tetrahydropyran-2-yl]phenyl]propyl]benzene-1,3-dicarboxamide

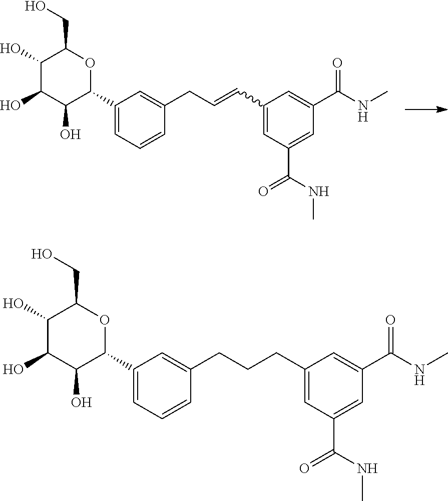 [SMILES: CNC(=O)c1cc(C=CCc2cccc([C@H]3O[C@H](CO)[C@@H](O)[C@H](O)[C@@H]3O)c2)cc(C(=O)NC)c1 ; CNC(=O)c1cc(CCCc2cccc([C@H]3O[C@H](CO)[C@@H](O)[C@H](O)[C@@H]3O)c2)cc(C(=O)NC)c1]

A mixture of COMPOUND 94 (9 mg, 0.0157 mmol) and Pd on C, wet, Degussa (18 mg, 0.017 mmol) in methanol (3 mL) is hydrogenated at 40 psi for 5 hours, filtered off catalyst, concentrated, dissolved in water-acetonitrile and freeze dried to afford title compound (4 mg, 48.6%).

¹H NMR (400 MHz, CD₃OD) δ 7.97 (t, 1H), 7.69 (d, J=1.6 Hz, 2H), 7.26 (brs, 1H), 7.19 (d, J=4.6 Hz, 2H), 7.06-7.01 (m, 1H), 4.86 (d, J=3.4 Hz, 1H), 4.35 (t, J=3.3 Hz, 1H), 3.72 (d, J=4.5 Hz, 2H), 3.63 (t, J=8.2 Hz, 1H), 3.48 (dd, J=8.2, 3.1 Hz, 1H), 3.41-3.32 (m, 1H), 2.83 (s, 3H), 2.71-2.55 (m, 4H), 1.99-1.84 (m, 2H). LC-MS: m/z=473.47 (M+H⁺).

### Preparation of Compound 98

### (2R,3S,4R,5S,6R)-2-[3-[3-(3,5-Dichlorophenyl)propyl]phenyl]-6-(hydroxymethyl)tetrahydropyran-3,4,5-triol

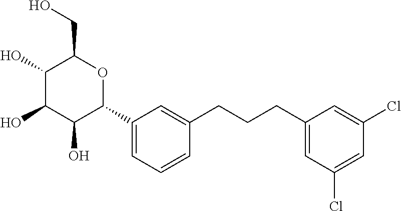 [SMILES: OC[C@H]1O[C@H](c2cccc(CCCc3cc(Cl)cc(Cl)c3)c2)[C@@H](O)[C@@H](O)[C@@H]1O]

A mixture of COMPOUND 95 (9 mg, 0.0157 mmol) and SiliaCat-Pd (43 mg, 0.05 mmol/g) in MeOH (3 mL) is stirred under one atmosphere of H₂ for 5 hours The catalyst is filtered off, concentrated, dissolved in water-acetonitrile and freeze dried to afford title compound.

¹H NMR (400 MHz, CD₃OD) δ 7.27-7.11 (m, 4H), 7.09-6.99 (m, 3H), 4.86 (d, J=2.9 Hz, 1H), 4.34 (t, 1H), 3.73 (d, J=4.6 Hz, 2H), 3.64 (t, J=8.0 Hz, 1H), 3.52-3.46 (m, 1H), 3.41-3.31 (m, 1H), 2.55 (dt, J=12.3, 7.8 Hz, 4H), 1.91-1.75 (m, 2H).

### Preparation of Compound 99

### 2R,3S,4R,5S,6R)-2-(Hydroxymethyl)-6-[3-(3-phenylpropyl)phenyl]tetrahydropyran-3,4,5-triol

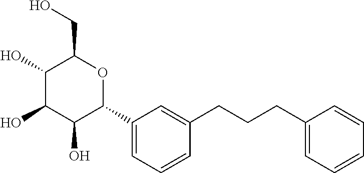 [SMILES: OC[C@H]1O[C@H](c2cccc(CCCc3ccccc3)c2)[C@@H](O)[C@@H](O)[C@@H]1O]

The title compound is prepared from COMPOUND 96 as described for COMPOUND 97.

¹H NMR (400 MHz, CD₃OD) δ 7.27-6.96 (m, 9H), 4.86 (d, J=3.3 Hz, 1H), 4.35 (t, J=3.2 Hz, 1H), 3.72 (d, J=4.6 Hz, 2H), 3.64 (t, J=8.1 Hz, 1H), 3.49 (dd, J=8.2, 3.0 Hz, 1H), 3.40-3.33 (m, 1H), 2.60-2.48 (m, 4H), 1.90-1.77 (m, 2H). LC-MS: m/z=381.21 (M+Na)⁺.

### Preparation of Compound 100

### Methyl 4-[2-[4-[(2R,3S,4R,5S,6R)-3,4,5-trihydroxy-6-(hydroxymethyl)tetrahydropyran-2-yl]phenyl]ethyl]benzoate

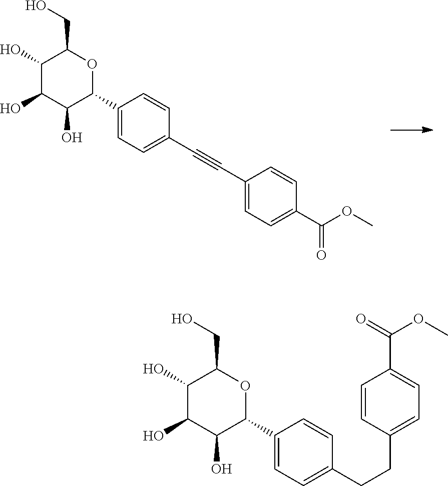 [SMILES: COC(=O)c1ccc(C#Cc2ccc([C@H]3O[C@H](CO)[C@@H](O)[C@H](O)[C@@H]3O)cc2)cc1 ; COC(=O)c1ccc(CCc2ccc([C@H]3O[C@H](CO)[C@@H](O)[C@H](O)[C@@H]3O)cc2)cc1]

The title compound is prepared from COMPOUND 89 as described for COMPOUND 97

LC-MS: m/z=403.44 (M+H⁺).

### Preparation of Compound 101

### Methyl 3-[2-[3-[(2R,3S,4R,5S,6R)-3,4,5-trihydroxy-6-(hydroxymethyl)tetrahydropyran-2-yl]phenyl]ethyl]benzoate

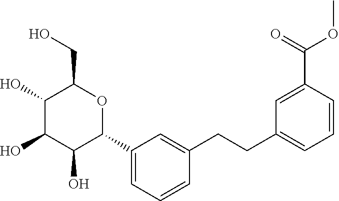 [SMILES: COC(=O)c1cccc(CCc2cccc([C@H]3O[C@H](CO)[C@@H](O)[C@H](O)[C@@H]3O)c2)c1]

The title compound is prepared from COMPOUND 86 as described for COMPOUND 97.

¹H NMR (400 MHz, CD₃OD) δ 7.84-7.76 (m, 2H), 7.42-7.20 (m, 5H), 7.07 (d, J=6.3 Hz, 1H), 4.92 (d, J=3.2 Hz, 1H), 4.41 (t, J=3.2 Hz, 1H), 3.87 (s, 3H), 3.78 (d, J=4.6 Hz, 2H), 3.71 (t, J=8.3 Hz, 1H), 3.52 (dd, J=8.3, 3.1 Hz, 1H), 3.42-3.34 (m, 1H), 3.02-2.88 (m, 4H). LC-MS: m/z=403.4 (M+H⁺).

### Preparation of Compound 102

### (2R,3S,4R,5S,6R)-2-(Hydroxymethyl)-6-[3-(2-phenylethyl)phenyl]tetrahydropyran-3,4,5-triol

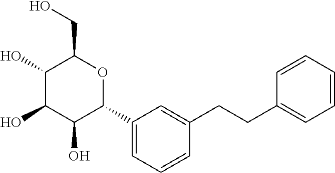 [SMILES: OC[C@H]1O[C@H](c2cccc(CCc3ccccc3)c2)[C@@H](O)[C@@H](O)[C@@H]1O]

The title compound is prepared from COMPOUND 91 as described for COMPOUND 97

¹H NMR (400 MHz, CD₃OD) δ 7.32-7.03 (m, 9H), 4.93 (d, J=3.3 Hz, 1H), 4.41 (t, J=3.2 Hz, 1H), 3.79 (d, J=4.6 Hz, 2H), 3.72 (t, J=8.3 Hz, 1H), 3.54 (dd, J=8.3, 3.1 Hz, 1H), 3.44-3.37 (m, 1H), 2.93-2.84 (m, 4H).

### Preparation of Compound 103

### Methyl 4-[2-[3-[(2R,3S,4R,5S,6R)-3,4,5-trihydroxy-6-(hydroxymethyl)tetrahydropyran-2-yl]phenyl]ethyl]benzoate

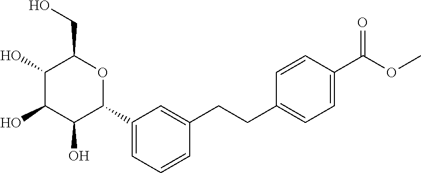 [SMILES: COC(=O)c1ccc(CCc2cccc([C@H]3O[C@H](CO)[C@@H](O)[C@H](O)[C@@H]3O)c2)cc1]

The title compound is prepared from COMPOUND 100 as described for COMPOUND 97

¹H NMR (400 MHz, CD₃OD) δ 7.84-7.75 (m, 2H), 7.23-7.13 (m, 5H), 7.02-6.96 (m, 1H), 4.84 (d, J=3.3 Hz, 1H), 4.32 (t, J=3.2 Hz, 1H), 3.78 (s, 3H), 3.70 (d, J=4.6 Hz, 2H), 3.62 (t, J=8.3 Hz, 1H), 3.43 (dd, J=8.3, 3.1 Hz, 1H), 3.33-3.25 (m, 1H), 2.95-2.80 (m, 4H). LC-MS: m/z=403.4 (M+H⁺).

### Preparation of Compound 104

### (2R,3S,4R,5S,6R)-2-(Hydroxymethyl)-6-[2-methyl-3-[4-(5-methyl-1,3,4-oxadiazol-2-yl)phenyl]phenyl]tetrahydropyran-3,4,5-triol

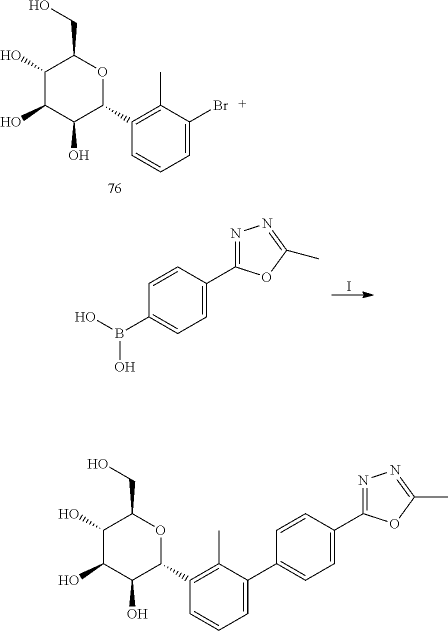

In 10 mL microwave vial, to a solution of COMPOUND 76 (31.0 mg, 0.09 mmol) and [4-(5-methyl-1,3,4-oxadiazol-2-yl)phenyl]boronic acid (28.5 mg, 0.14 mmol) in toluene (2.5 mL) and methanol (0.5 mL) is added powdered K₃PO₄ (39.5 mg, 0.186 mmol) in one portion, degassed (house vacuum/N₂ flush), Pd(PPh₃)₄ (16.1 mg, 0.014 mmol) is added in one portion and sealed and heated at 95° C. overnight, filtered through 0.4 micron filter, concentrated and purified by reverse phase HPLC to afford the title compound (24 mg, 61.4%) as white solid

¹H NMR (400 MHz, CD₃OD) δ 8.07 (d, J=8.3 Hz, 2H), 7.55 (d, J=7.5 Hz, 1H), 7.48 (d, J=8.3 Hz, 2H), 7.28 (t, J=7.7 Hz, 1H), 7.16 (d, J=6.7 Hz, 1H), 5.20 (d, J=7.0 Hz, 1H), 4.26 (dd, J=6.9, 3.3 Hz, 1H), 4.08-3.97 (m, 2H), 3.88-3.80 (m, 1H), 3.75 (dd, J=11.9, 3.8 Hz, 1H), 3.68-3.58 (m, 1H), 2.63 (s, 3H), 2.32 (s, 3H). LC-MS: m/z=413.37 (M+H⁺).

### Preparation of Compound 105

### (2R,3S,4R,5S,6R)-2-[2-Ethyl-3-[4-(5-methyl-1,3,4-oxadiazol-2-yl)phenyl]phenyl]-6-(hydroxymethyl)tetrahydropyran-3,4,5-triol

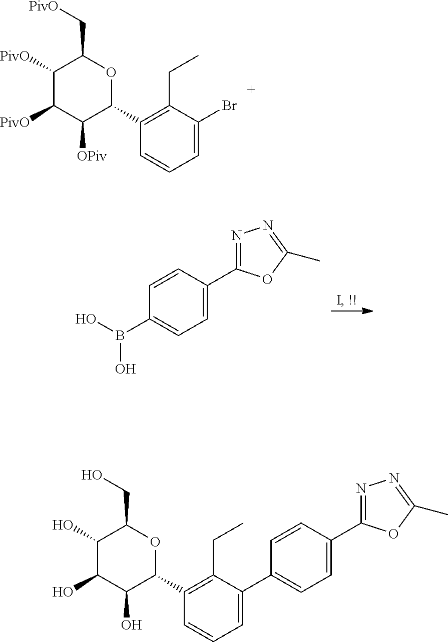

### Step I: [(2R,3R,4R,5R,6R)-6-(3-Bromo-2-ethyl-phenyl)-3,4,5-tris(2,2-dimethylpropanoyloxy)tetrahydropyran-2-yl]methyl 2,2-dimethylpropanoate

A solution of n-Bu₃MgLi (1.633 mL of 0.66 M, 1.078 mmol) in hexane heptane-dibutyl ether (8:20:3) is added to 1-bromo-2-ethyl-3-iodo-benzene (958 mg, 3.081 mmol) in toluene (1.5 mL) and dibutylether (0.9 mL) at 0° C., stirred at the same temperature for 3.5 h, a solution of ZnBr₂—LiBr in dibutyl ether (1.6 mL of 1.05 M, 1.67 mmol) is added drop wise, cooling bath is removed, stirred at room temperature for 1 h, a solution of [(2R,3R,4S,5S,6R)-6-bromo-3,4,5-tris(2,2-dimethylpropanoyloxy)tetrahydropyran-2-yl]methyl 2,2-dimethylpropanoate (1.49 g, 2.568 mmol) in toluene (2.7 mL) is added, it is placed on pre-heated oil bath at 90° C. for 18 hours. The reaction mixture is cooled to room temperature, poured into aq. 1N HCl solution, extracted with ethyl acetate, combined extracts are washed with brine, dried (Na₂SO₄), concentrated. The residue is purified on 25 g SNAP silica gel cartridge using ethyl acetate-hexanes (0% to 10%, 20 CV) as eluent afforded a mixture containing [(2R,3R,4R,5R,6R)-6-(3-bromo-2-ethyl-phenyl)-3,4,5-tris(2,2-dimethylpropanoyloxy)tetrahydropyran-2-yl]methyl 2,2-dimethylpropanoate (527 mg, 0.7708 mmol, 30.01%) as colorless foam. This material is used as such in the next step.

¹H NMR (400 MHz, CDCl₃) δ 7.49 (d, J=8.0 Hz, 1H), 7.42 (d, J=7.8 Hz, 1H), 7.07 (t, J=7.8 Hz, 1H), 5.52 (d, J=8.0 Hz, 1H), 5.48-5.42 (m, 1H), 5.22 (d, J=8.1 Hz, 1H), 5.16-5.09 (m, 1H), 4.76 (dd, J=11.5, 8.1 Hz, 1H), 4.16-4.04 (m, 2H), 3.05-2.82 (m, 2H), 1.26 (d, J=2.9 Hz, 18H), 1.15 (s, 12H), 0.96 (s, 9H)

### Step II: Compound 105

The title compound is prepared from [(2R,3R,4R,5R,6R)-6-(3-bromo-2-ethyl-phenyl)-3,4,5-tris(2,2-dimethylpropanoyloxy)tetrahydropyran-2-yl]methyl 2,2-dimethylpropanoate from Step I and [4-(5-methyl-1,3,4-oxadiazol-2-yl)phenyl]boronic acid using SiliaCat Pd at 100° C. in microwave as described for COMPOUND 79 followed by hydrolysis of the pivaloyl ester using NaOMe/MeOH at room temperature as described for COMPOUND 76.

¹H NMR (400 MHz, CD₃OD) δ 8.05 (d, J=8.4 Hz, 2H), 7.63 (d, J=7.8 Hz, 1H), 7.47 (d, J=8.4 Hz, 2H), 7.27 (t, J=7.7 Hz, 1H), 7.08 (dd, J=7.5, 1.2 Hz, 1H), 5.21 (d, J=8.5 Hz, 1H), 4.17 (dd, J=8.4, 3.1 Hz, 1H), 4.12-4.02 (m, 2H), 3.91 (dd, J=4.2, 2.3 Hz, 1H), 3.84-3.77 (m, 2H), 2.87-2.66 (m, 2H), 2.62 (s, 3H), 1.02 (t, J=7.5 Hz, 3H). LC-MS: m/z=427.24 (M+H⁺).

### Preparation of Compound 106

### (2R,3S,4R,5S,6R)-2-[2-Fluoro-3-[4-(5-methyl-1,3,4-oxadiazol-2-yl)phenyl]phenyl]-6-(hydroxymethyl)tetrahydropyran-3,4,5-triol

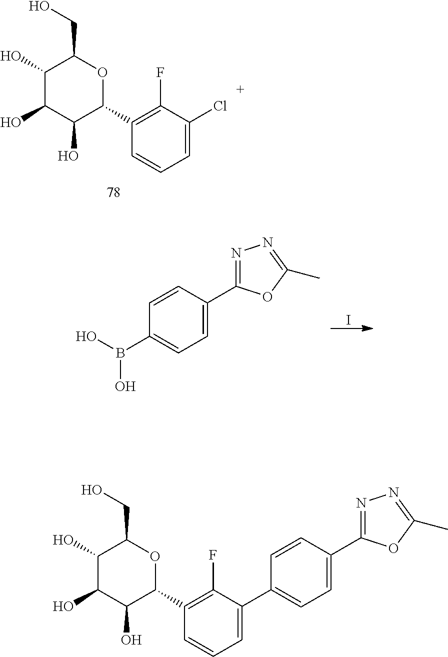

To a degassed mixture of [3-(2-dicyclohexylphosphanylphenyl)-2,4-dimethoxy-phenyl]sulfonyloxysodium (84.3 mg, 0.1644 mmol), COMPOUND 78 (50 mg, 0.1644 mmol), [3-(2-dicyclohexylphosphanylphenyl)-2,4-dimethoxy-phenyl]sulfonyloxysodium (84.3 mg, 0.164 mmol), [4-(5-methyl-1,3,4-oxadiazol-2-yl)phenyl]boronic acid (50.30 mg, 0.2466 mmol) and K₂CO₃ (114 mg, 0.82 mmol) in 2-Me THF (1.2 mL) and water (240 μL) is added Palladium (II) acetate (18.5 mg, 0.082 mmol), reaction mixture is slowly heated to 90° C., stirrer for 20 hours, filtered through celite, washed with 10% H₂O-methanol, concentrated, purified on 25 g C18 SNAP silica gel cartridge using acetonitrile in water (10% to 40%) as eluent, followed by further purification on reverse phase HPLC to afford the title compound (25 mg, 34.7%) as white solid.

¹H NMR (400 MHz, CD₃OD) δ 8.11-8.04 (m, 2H), 7.72 (dd, J=8.3, 1.3 Hz, 2H), 7.70-7.64 (m, 1H), 7.45 (td, J=7.5, 1.6 Hz, 1H), 7.30 (t, J=7.7 Hz, 1H), 5.23 (d, J=7.2 Hz, 1H), 4.19 (dd, J=7.2, 2.4 Hz, 1H), 4.11-4.01 (m, 1H), 3.90-3.74 (m, 4H), 2.62 (s, 3H). LC-MS: m/z=417.19 (M+H⁺).

### Preparation of Compound 107

### (2R,3S,4R,5S,6R)-2-(hydroxymethyl)-6-[3-[2-methoxy-4-(5-methyl-1,3,4-oxadiazol-2-yl)phenyl]-2-methyl-phenyl]tetrahydropyran-3,4,5-triol

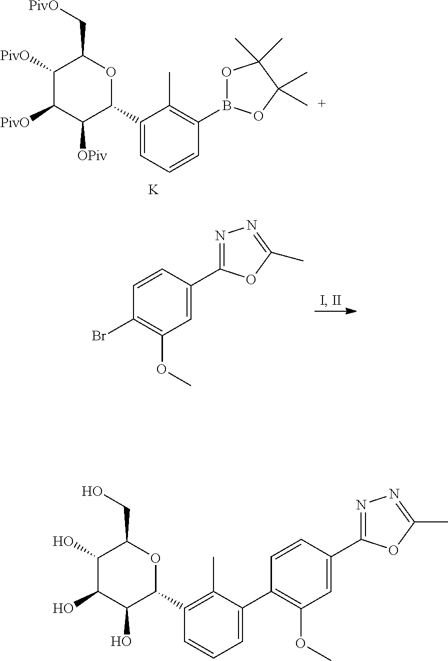

The title compound is prepared from INTERMEDIATE K and 2-(4-bromo-3-methoxyphenyl)-5-methyl-1,3,4-oxadiazole as described in COMPOUND 79.

¹H NMR (400 MHz, CD₃OD) δ 7.68-7.63 (m, 2H), 7.51 (d, J=7.8 Hz, 1H), 7.30-7.20 (m, 2H), 7.06 (d, J=7.6 Hz, 1H), 5.18 (d, J=5.3 Hz, 1H), 4.31-4.20 (m, 1H), 4.07-3.95 (m, 2H), 3.87-3.83 (m, 1H), 3.82 (s, 3H), 3.79-3.71 (m, 1H), 3.65-3.57 (m, 1H), 2.63 (s, 3H), 2.18 and 2.17 (two singlets, 3H). LC-MS: m/z=443.24 (M+H⁺).

### Preparation of Compound 108

### (2R,3S,4R,5S,6R)-2-(Hydroxymethyl)-6-[2-methyl-3-[4-(1-methylpyrazol-3-yl)phenyl]phenyl]tetrahydropyran-3,4,5-triol

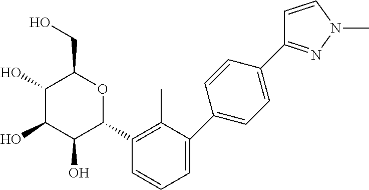 [SMILES: Cc1c(-c2ccc(-c3ccn(C)n3)cc2)cccc1[C@H]1O[C@H](CO)[C@@H](O)[C@H](O)[C@@H]1O]

The title compound is prepared from INTERMEDIATE K and 3-(4-bromophenyl)-1-methyl-1H-pyrazole as described in COMPOUND 79.

¹H NMR (400 MHz, CD₃OD) δ 7.71 (d, J=8.2 Hz, 2H), 7.53 (d, J=2.2 Hz, 1H), 7.42 (d, J=7.4 Hz, 1H), 7.21 (d, J=8.2 Hz, 2H), 7.16 (t, J=7.7 Hz, 1H), 7.07 (d, J=6.9 Hz, 1H), 6.56 (d, J=2.3 Hz, 1H), 5.11 (d, J=6.8 Hz, 1H), 4.19 (dd, J=6.7, 3.2 Hz, 1H), 3.98-3.89 (m, 2H), 3.85 (s, 3H), 3.79-3.73 (m, 1H), 3.68 (dd, J=11.9, 3.8 Hz, 1H), 3.59-3.49 (m, 1H), 2.25 (s, 3H). LC-MS: m/z=411.42 (M+H⁺).

### Preparation of Compound 109

### (2R,3S,4R,5S,6R)-2-(Hydroxymethyl)-6-[2-methyl-3-(4-methylsulfonylphenyl)phenyl]tetrahydropyran-3,4,5-triol

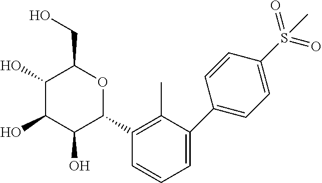 [SMILES: Cc1c(-c2ccc(S(C)(=O)=O)cc2)cccc1[C@H]1O[C@H](CO)[C@@H](O)[C@H](O)[C@@H]1O]

The title compound is prepared from INTERMEDIATE K and 1-bromo-4-(methylsulfonyl)benzene as described in COMPOUND 79.

¹H NMR (400 MHz, CD₃OD) δ 7.91 (d, J=8.3 Hz, 2H), 7.52-7.43 (m, 3H), 7.20 (t, J=7.7 Hz, 1H), 7.06 (d, J=7.4 Hz, 1H), 5.11 (d, J=7.1 Hz, 1H), 4.17 (dd, J=7.0, 3.2 Hz, 1H), 4.00-3.89 (m, 2H), 3.78-3.74 (m, 1H), 3.67 (dd, J=11.9, 3.7 Hz, 1H), 3.61-3.52 (m, 1H), 3.08 (s, 3H), 2.22 (s, 3H).

### Preparation of Compound 110

### (2R,3S,4R,5S,6R)-2-(Hydroxymethyl)-6-[2-methyl-3-(3-methylsulfonylphenyl)phenyl]tetrahydropyran-3,4,5-triol

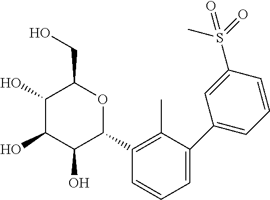 [SMILES: Cc1c(-c2cccc(S(C)(=O)=O)c2)cccc1[C@H]1O[C@H](CO)[C@@H](O)[C@H](O)[C@@H]1O]

The title compound is prepared from INTERMEDIATE K and 1-bromo-3-(methylsulfonyl)benzene as described in COMPOUND 79.

¹H NMR (400 MHz, CD₃OD) δ 7.96-7.90 (m, 1H), 7.84 (t, J=1.6 Hz, 1H), 7.68 (t, J=7.6 Hz, 1H), 7.63 (dt, J=7.7, 1.4 Hz, 1H), 7.57 (d, J=7.2 Hz, 1H), 7.29 (t, J=7.7 Hz, 1H), 7.16 (d, J=6.6 Hz, 1H), 5.19 (d, J=7.1 Hz, 1H), 4.25 (dd, J=7.0, 3.2 Hz, 1H), 4.07-3.98 (m, 2H), 3.84 (dd, J=5.4, 4.2 Hz, 1H), 3.75 (dd, J=11.9, 3.8 Hz, 1H), 3.68-3.59 (m, 1H), 3.15 (s, 3H), 2.29 (s, 3H). LC-MS: m/z=409.24 (M+H⁺).

### Preparation of Compound 111

### (2R,3S,4R,5S,6R)-2-(Hydroxymethyl)-6-[2-methyl-3-[3-methyl-4-(5-methyl-1,3,4-oxadiazol-2-yl)phenyl]phenyl]tetrahydropyran-3,4,5-triol

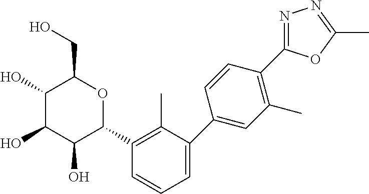 [SMILES: Cc1nnc(-c2ccc(-c3cccc([C@H]4O[C@H](CO)[C@@H](O)[C@H](O)[C@@H]4O)c3C)cc2C)o1]

The title compound is prepared from INTERMEDIATE K and 2-(4-bromo-2-methylphenyl)-5-methyl-1,3,4-oxadiazole (see preparation below) as described in COMPOUND 105.

¹H NMR (400 MHz, CD₃OD) δ 7.94 (d, J=8.0 Hz, 1H), 7.54 (d, J=7.1 Hz, 1H), 7.32 (s, 1H), 7.30-7.23 (m, 2H), 7.17-7.12 (m, 1H), 5.19 (d, J=6.9 Hz, 1H), 4.26 (dd, J=6.9, 3.3 Hz, 1H), 4.06-3.97 (m, 2H), 3.86-3.81 (m, 1H), 3.76 (dd, J=11.9, 3.8 Hz, 1H), 3.67-3.61 (m, 1H), 2.68 (s, 3H), 2.63 (s, 3H), 2.32 (s, 3H). LC-MS: m/z=426.89 (M+H⁺).

### Preparation of 2-(4-bromo-2-methyl-phenyl)-5-methyl-1,3,4-oxadiazole

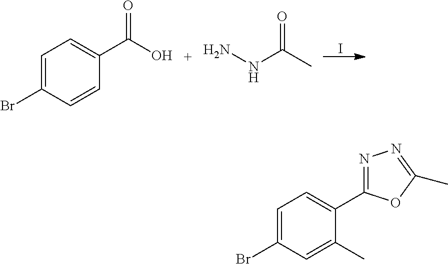

A mixture of acetohydrazide (500 mg, 6.749 mmol), 4-bromo-2-methyl-benzoic acid (1.451 g, 6.749 mmol) in POCl₃ (5 mL, 53.64 mmol) is refluxed as described for the preparation of the 1-[5-(4-bromophenyl)-1,3,4-oxadiazol-2-yl]-N,N-dimethyl-methanamine of step I followed by purification to afford 2-(4-bromo-2-methyl-phenyl)-5-methyl-1,3,4-oxadiazole (680 mg, 2.687 mmol, 39.81%) as white solid.

¹H NMR (400 MHz, CDCl₃) δ 7.76 (d, J=8.4 Hz, 1H), 7.52-7.49 (m, 1H), 7.45 (d, J=8.7 Hz, 1H), 2.68 (s, 3H), 2.63 (s, 3H).

Ref: Gaster, L. et al. PCT Int. Appl. (1996), WO9619477A119960627.

### Preparation of Compound 112

### (2R,3S,4R,5S,6R)-2-(Hydroxymethyl)-6-[3-[3-methoxy-4-(5-methyl-1,3,4-oxadiazol-2-yl)phenyl]-2-methyl-phenyl]tetrahydropyran-3,4,5-triol

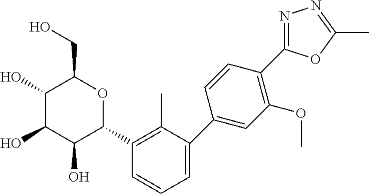 [SMILES: COc1cc(-c2cccc([C@H]3O[C@H](CO)[C@@H](O)[C@H](O)[C@@H]3O)c2C)ccc1-c1nnc(C)o1]

The title compound is prepared from INTERMEDIATE K and 2-(4-bromo-2-methoxyphenyl)-5-methyl-1,3,4-oxadiazole as described in COMPOUND 79.

LC-MS: m/z=443.3 (M+H⁺).

### Preparation of Compound 113

### (2R,3S,4R,5S,6R)-2-[3-[4-[5-[(Dimethylamino)methyl]-1,3,4-oxadiazol-2-yl]phenyl]phenyl]-6-(hydroxymethyl)tetrahydropyran-3,4,5-triol

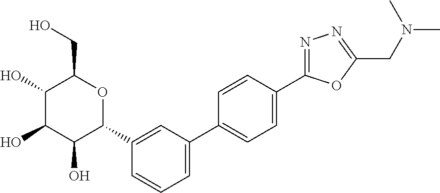 [SMILES: CN(C)Cc1nnc(-c2ccc(-c3cccc([C@H]4O[C@H](CO)[C@@H](O)[C@H](O)[C@@H]4O)c3)cc2)o1]

The title compound is prepared from INTERMEDIATE J and 1-(5-(4-bromophenyl)-1,3,4-oxadiazol-2-yl)-N,N-dimethylmethanamine (see preparation below) according to the procedure described for COMPOUND 106 followed by deacetylation using NaOMe/MeOH.

¹H NMR (400 MHz, CD₃OD) δ 8.05 (d, J=8.5 Hz, 2H), 7.84-7.77 (m, 2H), 7.56 (d, J=6.6 Hz, 1H), 7.47-7.38 (m, 2H), 4.95 (d, J=4.0 Hz, 1H), 4.42-4.34 (m, 1H), 3.82 (s, 2H), 3.79-3.75 (m, 2H), 3.66 (t, J=7.6 Hz, 1H), 3.55 (dd, J=7.7, 3.1 Hz, 1H), 3.51-3.43 (m, 1H), 2.32 (s, 6H). LC-MS: m/z=442.26 (M+H⁺).

### Preparation of 1-[5-(4-bromophenyl)-1,3,4-oxadiazol-2-yl]-N,N-dimethyl-methanamine

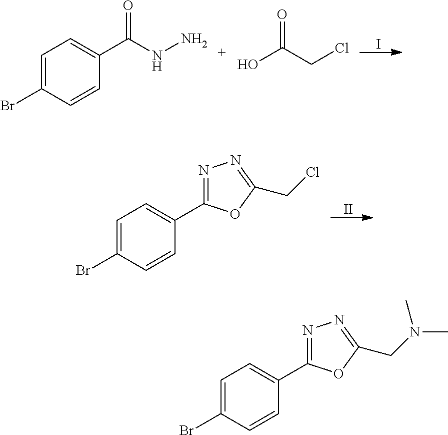

### Step I: 2-(4-bromophenyl)-5-(chloromethyl)-1,3,4-oxadiazole

A mixture of 4-bromobenzohydrazide (6.451 g, 30 mmol), 2-chloroacetic acid (2.835 g, 30.00 mmol) in POCl₃ (21 mL, 225.3 mmol) is slowly heated to reflux, suspension became clear light brown solution, it is refluxed for 2.5 hour (Padmavathi, V. et. al. Eur. J. Med. Chem. 2011, 46, 1367), cooled to room temperature, excess POCl₃ is removed on rotavaporator, the syrup is poured into crushed ice, it is extracted with ˜20% EtOAC in methylene chloride (3×60 mL), combined extracts are washed with bicarbonate solution (until basic pH), dried, concentrated, triturated twice with methylene chloride (5 mL) to afford 2-(4-bromophenyl)-5-(chloromethyl)-1,3,4-oxadiazole (4.3 g, 52.4%) as light brown solid. Mother liquid is concentrated and purified on 50 g SNAP silica gel cartridge using a gradient of ethyl acetate in hexanes (15% to 50%) as eluent to afford additional product (2.2 g, 26.8%) as light yellow solid. For stepwise procedure see, Daniel, et. al. PCT Int. Appl., 2005121152 A1, 22 Dec. 2005

### Step II: 1-(5-(4-bromophenyl)-1,3,4-oxadiazol-2-yl)-N,N-dimethylmethanamine (See Preparation Below)

A mixture of 2-(4-bromophenyl)-5-(chloromethyl)-1,3,4-oxadiazole (500 mg, 1.828 mmol) in dimethylammonium in ethanol (5 mL of 33% w/v, 15.26 mmol) is heated in microwave vial at 100° C. for 20 min (TLC showed complete consumption of the starting material), filtered through 0.4 micron filter, concentrated, diluted with methanol and Et₃N (0.5 mL) is added, loaded onto C18 samplet, purified on 50 G C18 SNAP silica gel cartridge using acetonitrile in water (10% to 55%) as eluent to afford title compound (422 mg, 81.8%) as colorless oil.

1H NMR (400 MHz, CDCl₃) δ 7.99-7.91 (m, 2H), 7.70-7.63 (m, 2H), 3.83 (s, 2H), 2.40 (s, 6H).

### Preparation of Compound 114

### Trimethyl-[[5-[4-[3-[(2R,3S,4R,5S,6R)-3,4,5-trihydroxy-6-(hydroxymethyl)tetrahydropyran-2-yl]phenyl]phenyl]-1,3,4-oxadiazol-2-yl]methyl]ammonium

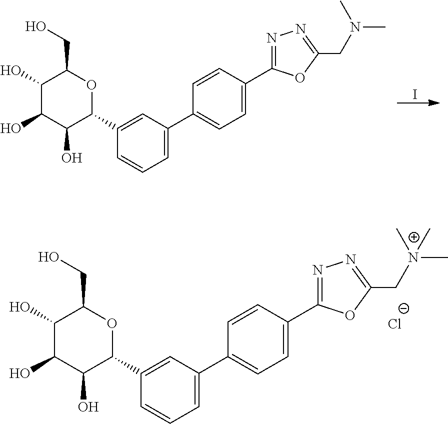

To a stirred solution of COMPOUND 113 (7 mg, 0.016 mmol) in DMF (0.4 mL) is added iodomethane (20 μL, 0.321 mmol), reaction mixture is stirred at room temperature over weekend, diluted with aq. 1N HCl (0.4 mL), stirred for 15 min, purified on 12 g C-18 SNAP silica gel cartridge using gradient of acetonitrile-water (10% to 30%), freeze dried to afford the title compound.

¹H NMR (400 MHz, CD₃OD) δ 8.18 (d, J=8.5 Hz, 2H), 7.92-7.85 (m, 3H), 7.64 (d, J=6.6 Hz, 1H), 7.53-7.47 (m, 2H), 5.13 (s, 2H), 5.03 (d, J=3.9 Hz, 1H), 4.47 (t, J=3.5 Hz, 1H), 3.89-3.80 (m, 2H), 3.75 (t, J=7.6 Hz, 1H), 3.68-3.61 (m, 1H), 3.59-3.51 (m, 1H), 3.39 (s, 9H). LC-MS: m/z=456.65 (M+H⁺).

### Preparation of Compound 115

### tert-Butyl N-[[5-[4-[3-[(2R,3S,4R,5S,6R)-3,4,5-trihydroxy-6-(hydroxymethyl)tetrahydropyran-2-yl]phenyl]phenyl]-1,3,4-oxadiazol-2-yl]methyl]carbamate

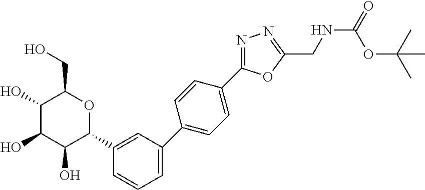 [SMILES: CC(C)(C)OC(=O)NCc1nnc(-c2ccc(-c3cccc([C@H]4O[C@H](CO)[C@@H](O)[C@H](O)[C@@H]4O)c3)cc2)o1]

The title compound is prepared from INTERMEDIATE J and tert-butyl N-[[5-(4-bromophenyl)-1,3,4-oxadiazol-2-yl]methyl]-N-tert-butoxycarbonyl-carbamate according to the procedure described for COMPOUND 106 followed by deacetylation using NaOMe/MeOH.

¹H NMR (400 MHz, CD₃OD) δ 8.07 (d, J=8.2 Hz, 2H), 7.88-7.81 (m, 3H), 7.61 (d, J=6.9 Hz, 1H), 7.53-7.45 (m, 2H), 5.02 (d, J=3.9 Hz, 1H), 4.54 (s, 2H), 4.46 (t, J=3.5 Hz, 1H), 3.89-3.83 (m, 2H), 3.75 (t, J=7.6 Hz, 1H), 3.64 (dd, J=7.7, 3.0 Hz, 1H), 3.59-3.50 (m, 1H), 1.46 (s, 9H). LC-MS: m/z=514.32 (M+H⁺).

### Preparation of tert-butyl N-[[5-(4-bromophenyl)-1,3,4-oxadiazol-2-yl]methyl]-N-tert-butoxycarbonyl-carbamate and tert-butyl N-[[5-(4-bromophenyl)-1,3,4-oxadiazol-2-yl]methyl]carbamate

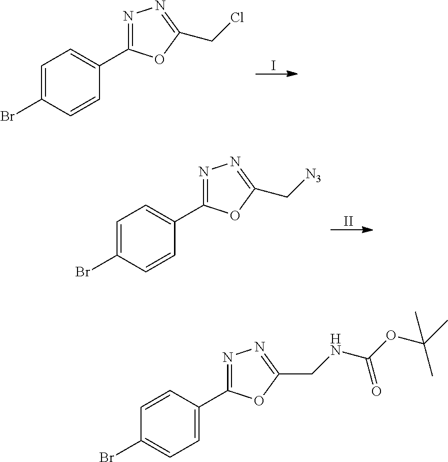

### Step I: 2-(azidomethyl)-5-(4-bromophenyl)-1,3,4-oxadiazole

To a stirred solution of 2-(4-bromophenyl)-5-(chloromethyl)-1,3,4-oxadiazole (1000 mg, 3.656 mmol) in DMSO (10 mL) is added sodium azide (713.0 mg, 10.97 mmol), reaction mixture is heated at 60° C. for 1.5 hour, cooled to room temperature, diluted with water (20 mL), extracted with ethyl acetate (2×30 mL), combined extracts are washed with brine, dried (Na₂SO₄), concentrated, purification of the residue on 50 g SNAP silica gel cartridge using ethyl acetate-hexanes (15% to 40%, 8 CV; and then 40%) as eluent afforded the title compound (900 mg, 87.9%).

¹H NMR (400 MHz, CDCl₃) δ 7.99-7.90 (m, 2H), 7.71-7.61 (m, 2H), 4.64 (s, 2H).

### Step II: tert-butyl N-[[5-(4-bromophenyl)-1,3,4-oxadiazol-2-yl]methyl]carbamate

To a stirred solution of 2-(azidomethyl)-5-(4-bromophenyl)-1,3,4-oxadiazole (600 mg, 2.142 mmol) in THF (6.0 mL) and water (667 μL) is added PPh₃ (590 mg, 2.249 mmol) in one portion (Nitrogen gas evolution is occurred after few minutes, exothermic), reaction mixture is stirred at room temperature for 2.5 hours, concentrated, dissolved in CH₂Cl₂ (6.0 mL), Et₃N (542 mg, 746.0 μL, 5.355 mmol), tert-butoxycarbonyl tert-butyl carbonate (608 mg, 2.785 mmol) and N,N-dimethylpyridin-4-amine (17 mg, 0.1392 mmol) is added sequentially, resultant light yellow suspension is stirred overnight, concentrated, diluted with water (10 mL) and methylene chloride (10 mL), organic solution is separated on phase separator, aqueous solution is rinsed with methylene chloride (2×), combined filtrate is concentrated, purified on 40 g Silica gel cartridge using ethyl acetate in hexanes (15% to 50%) to afford the title compound (170 mg, 22.4%) as white solid.

¹H NMR (400 MHz, CDCl₃) δ 7.93-7.86 (m, 2H), 7.69-7.62 (m, 2H), 5.33 (s, 1H), 4.64 (d, J=5.9 Hz, 2H), 1.48 (s, 9H).

### Preparation of Compound 117

### (2R,3S,4R,5S,6R)-2-(hydroxymethyl)-6-[3-[4-(5-methyloxazol-2-yl)phenyl]phenyl]tetrahydropyran-3,4,5-triol

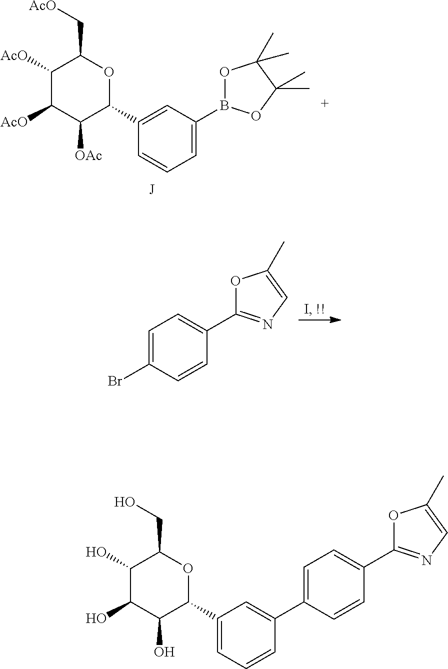

A microwave vial is charged with (2R,3R,4R,5R,6R)-2-(acetoxymethyl)-6-(3-(4,4,5,5-tetramethyl-1,3,2-dioxaborolan-2-yl)phenyl)tetrahydro-2H-pyran-3,4,5-triyl triacetate (1 eq.), the 2-(4-bromophenyl)-5-methyloxazole (1 eq.), Siliacat-DPP-Pd™ (0.26 mmol/g, 0.1 eq.), Cs₂CO₃ (2.2 eq.) and acetonitrile. The mixture is heated in the microwaved for 30 minutes at 100° C., filtered on Celite and concentrated to dryness. The residue is dissolved in MeOH, treated with 0.5 M NaOMe (0.5 eq.) and stirred overnight at room temperature. The mixture is concentrated and loaded onto a cation-exchange resin (SXC, cartridge, 1 g). The column is rinsed with methanol for the equivalent of 4 CV. The mixture is concentrated to dryness and purified by reverse phase preparative HPLC to give the desired product.

¹H NMR (400 MHz, CD₃OD) δ 7.95 (d, J=8.5 Hz, 2H), 7.84-7.67 (m, 3H), 7.58-7.50 (m, J=3.7 Hz, 1H), 7.49-7.35 (m, 2H), 6.84 (d, J=1.2 Hz, 1H), 4.95 (d, J=3.8 Hz, 1H), 4.39 (t, 1H), 3.80-3.74 (m, 1H), 3.66 (t, J=7.7 Hz, 1H), 3.55 (dd, J=7.8, 3.1 Hz, 1H), 3.50-3.41 (m, 1H), 2.34 (d, J=1.1 Hz, 3H). LC-MS: m/z=398.33 (M+H+)

### Preparation of Compound 118

### (2R,3S,4R,5S,6R)-2-(hydroxymethyl)-6-[3-[4-(5-isopropyl-1,3,4-oxadiazol-2-yl)phenyl]phenyl]tetrahydropyran-3,4,5-triol

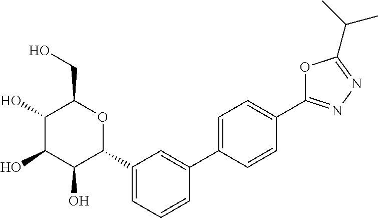 [SMILES: CC(C)c1nnc(-c2ccc(-c3cccc([C@H]4O[C@H](CO)[C@@H](O)[C@H](O)[C@@H]4O)c3)cc2)o1]

The title compound is prepared from INTERMEDIATE J and 2-(4-bromophenyl)-5-isopropyl-1,3,4-oxadiazole according to the procedure described for COMPOUND 117.

¹H NMR (400 MHz, CD₃OD) δ 8.10 (d, J=8.0 Hz, 2H), 7.91-7.84 (m, J=7.4 Hz, 3H), 7.51 (s, 3H), 5.03 (s, 1H), 4.46 (s, 1H), 3.87-3.83 (m, J=6.2 Hz, 1H), 3.83-3.79 (m, 1H), 3.73 (dd, J=15.5, 7.8 Hz, 3H), 3.63 (d, J=5.3 Hz, 1H), 3.61-3.52 (m, 3H), 1.46 (s, 3H), 1.45 (d, 6H). LC-MS: m/z=427.34 (M+H+)

### Preparation of Compound 119

### (2R,3S,4R,5S,6R)-2-(hydroxymethyl)-6-[3-(4-oxazol-5-ylphenyl)phenyl]tetrahydropyran-3,4,5-triol

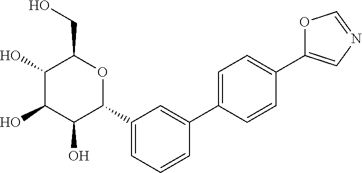 [SMILES: OC[C@H]1O[C@H](c2cccc(-c3ccc(-c4cnco4)cc3)c2)[C@@H](O)[C@@H](O)[C@@H]1O]

The title compound is prepared from INTERMEDIATE J and 5-(4-bromophenyl)oxazole according to the procedure described for COMPOUND 117.

¹H NMR (400 MHz, CD₃OD) δ 8.26 (s, 1H), 7.85-7.78 (m, 3H), 7.78-7.73 (m, J=8.3 Hz, 2H), 7.62-7.52 (m, J=17.1 Hz, 2H), 7.50-7.44 (m, J=4.5 Hz, 2H), 5.03 (d, J=3.5 Hz, 1H), 4.49-4.44 (m, 1H), 3.89-3.81 (m, J=6.3 Hz, 2H), 3.74 (t, J=7.6 Hz, 1H), 3.63 (dd, J=7.8, 2.8 Hz, 1H), 3.59-3.50 (m, J=6.5 Hz, 1H) LC-MS: m/z=384.32 (M+H+)

### Preparation of Compound 120

### (2R,3S,4R,5S,6R)-2-(hydroxymethyl)-6-[3-[4-[5-(2-pyridyl)-1,3,4-oxadiazol-2-yl]phenyl]phenyl]tetrahydropyran-3,4,5-triol

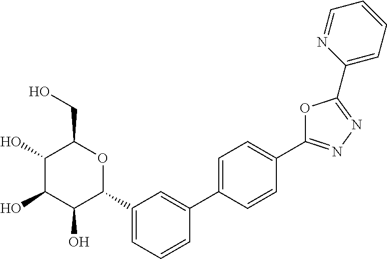 [SMILES: OC[C@H]1O[C@H](c2cccc(-c3ccc(-c4nnc(-c5ccccn5)o4)cc3)c2)[C@@H](O)[C@@H](O)[C@@H]1O]

The title compound is prepared from INTERMEDIATE J and 2-(4-bromophenyl)-5-(pyridin-2-yl)-1,3,4-oxadiazole according to the procedure described for COMPOUND 117.

¹H NMR (400 MHz, CD₃OD) δ 8.70 (d, J=4.8 Hz, 1H), 8.28-8.15 (m, J=20.9, 8.2 Hz, 3H), 8.00 (td, J=7.8, 1.7 Hz, 1H), 7.90-7.77 (m, 3H), 7.62-7.50 (m, J=9.8, 5.0 Hz, 2H), 7.49-7.36 (m, 2H), 4.96 (d, J=4.0 Hz, 1H), 4.42-4.35 (m, 1H), 3.85-3.71 (m, 2H), 3.71-3.62 (m, J=7.6 Hz, 1H), 3.62-3.53 (m, J=7.7, 3.1 Hz, 1H), 3.52-3.43 (m, J=6.9, 3.4 Hz, 1H). LC-MS: m/z=462.42 (M+H+)

### Preparation of Compound 121

### (2R,3S,4R,5S,6R)-2-(hydroxymethyl)-6-[3-[4-[5-(5-methyl-2-furyl)-1,3,4-oxadiazol-2-yl]phenyl]phenyl]tetrahydropyran-3,4,5-triol

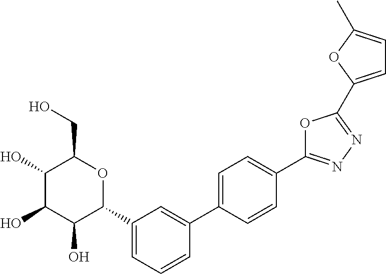 [SMILES: Cc1ccc(-c2nnc(-c3ccc(-c4cccc([C@H]5O[C@H](CO)[C@@H](O)[C@H](O)[C@@H]5O)c4)cc3)o2)o1]

The title compound is prepared from INTERMEDIATE J and 2-(4-bromophenyl)-5-(5-methylfuran-2-yl)-1,3,4-oxadiazole according to the procedure described for COMPOUND 117.

¹H NMR (400 MHz, CD₃OD) δ 8.17 (d, J=8.5 Hz, 2H), 7.93-7.81 (m, 3H), 7.67-7.58 (m, J=6.6 Hz, 1H), 7.56-7.43 (m, 2H), 7.27 (d, J=3.4 Hz, 1H), 6.36 (dd, J=3.4, 0.9 Hz, 1H), 5.03 (d, J=4.0 Hz, 1H), 4.48-4.39 (m, 1H), 3.93-3.80 (m, 2H), 3.75 (t, J=7.6 Hz, 1H), 3.64 (dd, J=7.7, 3.1 Hz, 1H), 3.56 (td, J=6.9, 3.4 Hz, 1H), 2.45 (s, 3H). LC-MS: m/z=465.34 (M+H+)

### Preparation of Compound 122

### (2R,3S,4R,5S,6R)-2-(hydroxymethyl)-6-[3-[4-(1-methylpyrazol-3-yl)phenyl]phenyl]tetrahydropyran-3,4,5-triol

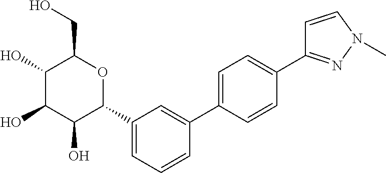 [SMILES: Cn1ccc(-c2ccc(-c3cccc([C@H]4O[C@H](CO)[C@@H](O)[C@H](O)[C@@H]4O)c3)cc2)n1]

The title compound is prepared from INTERMEDIATE J and 3-(4-bromophenyl)-1-methyl-1H-pyrazole according to the procedure described for COMPOUND 117.

¹H NMR (400 MHz, CD₃OD) δ 7.86-7.77 (m, J=6.4 Hz, 3H), 7.68 (d, J=8.4 Hz, 2H), 7.62-7.54 (m, 2H), 7.48-7.40 (m, 2H), 6.64 (d, J=2.3 Hz, 1H), 5.03 (d, J=3.7 Hz, 1H), 4.49 (t, J=3.4 Hz, 1H), 3.93 (s, 3H), 3.85-3.82 (m, 2H), 3.74 (t, J=7.9 Hz, 1H), 3.64 (dd, J=8.0, 3.2 Hz, 1H), 3.59-3.50 (m, 1H). LC-MS: m/z=397.34 (M+H+).

### Preparation of Compound 123

### (2R,3S,4R,5S,6R)-2-(hydroxymethyl)-6-[3-[3-(5-methyl-1,3,4-oxadiazol-2-yl)phenyl]phenyl]tetrahydropyran-3,4,5-triol

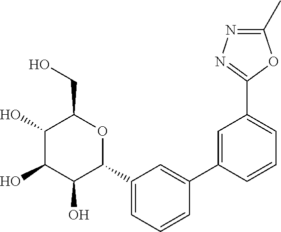 [SMILES: Cc1nnc(-c2cccc(-c3cccc([C@H]4O[C@H](CO)[C@@H](O)[C@H](O)[C@@H]4O)c3)c2)o1]

The title compound is prepared from INTERMEDIATE J and 2-(3-bromophenyl)-5-methyl-1,3,4-oxadiazole according to the procedure described for COMPOUND 117.

¹H NMR (400 MHz, CD₃OD) δ 8.31-8.27 (m, J=1.6 Hz, 1H), 8.04-7.95 (m, 1H), 7.93-7.80 (m, 2H), 7.70-7.57 (m, J=9.2, 8.3, 6.1 Hz, 2H), 7.55-7.44 (m, 2H), 5.03 (d, J=4.1 Hz, 1H), 4.51-4.39 (m, 1H), 3.93-3.81 (m, 2H), 3.75 (t, J=7.6 Hz, 1H), 3.64 (dd, J=7.7, 3.1 Hz, 1H), 3.59-3.50 (m, J=6.9, 3.3 Hz, 1H), 2.63 (d, J=3.6 Hz, 2H). LC-MS: m/z=399.34 (M+H+).

### Preparation of Compound 124

### (2R,3S,4R,5S,6R)-2-(hydroxymethyl)-6-[3-[4-(2-methylpyrazol-3-yl)phenyl]phenyl]tetrahydropyran-3,4,5-triol

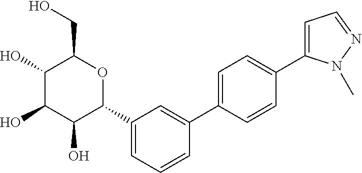 [SMILES: Cn1nccc1-c1ccc(-c2cccc([C@H]3O[C@H](CO)[C@@H](O)[C@H](O)[C@@H]3O)c2)cc1]

The title compound is prepared from INTERMEDIATE J and 5-(4-bromophenyl)-1-methyl-1H-pyrazole according to the procedure described for COMPOUND 117.

¹H NMR (400 MHz, CD₃OD) δ 7.84 (s, 1H), 7.78 (d, J=8.3 Hz, 2H), 7.63-7.53 (m, J=15.7, 6.1 Hz, 3H), 7.51-7.44 (m, J=8.3, 3.5 Hz, 3H), 6.40 (d, J=2.0 Hz, 1H), 5.04 (d, J=3.9 Hz, 1H), 4.60 (s, 1H), 4.48 (t, J=3.5 Hz, 1H), 3.90 (s, 3H), 3.88-3.81 (m, J=9.1, 5.9 Hz, 2H), 3.75 (t, J=7.8 Hz, 1H), 3.63 (dd, J=7.8, 3.1 Hz, 1H), 3.58-3.48 (m, 1H). LC-MS: m/z=397.45 (M+H+).

### Preparation of Compound 125

### (2R,3S,4R,5S,6R)-2-(hydroxymethyl)-6-[3-[2-methyl-4-(5-methyl-1,3,4-oxadiazol-2-yl)phenyl]phenyl]tetrahydropyran-3,4,5-triol

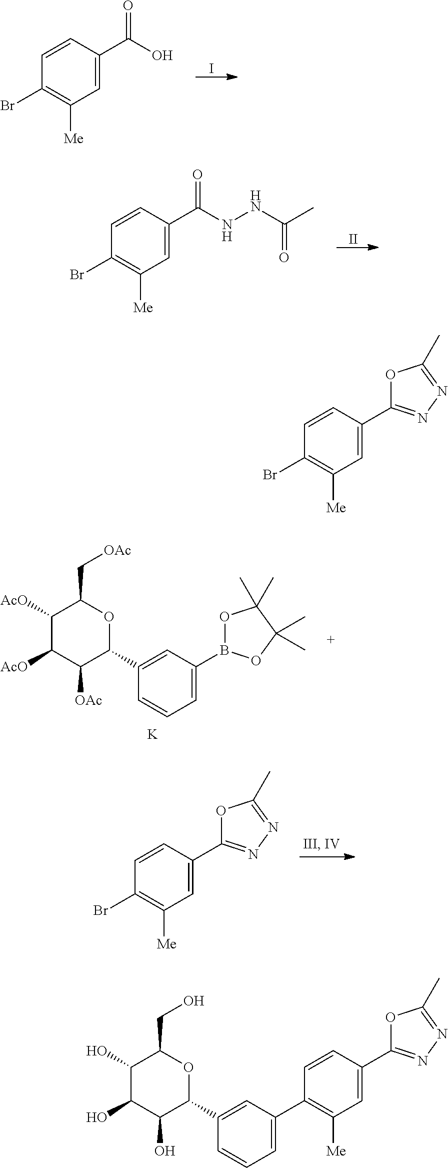

### Step I: N′-acetyl-4-bromo-3-methyl-benzohydrazide

To a solution of 4-bromo-3-methyl-benzoic acid (1.45 g, 6.75 mmol), acetohydrazide (500 mg, 6.75 mmol) in DMF (20 mL) are sequentially added HATU (3.08 g, 8.1 mmol), triethylamine (1.50 g, 2.07 mL, 14.85 mmol) and the mixture is stirred overnight at room temperature. Ethyl acetate (80 mL) and (20 mL) of water are added and the phases are separated. Aqueous phase is back extracted one time with EtOAc. The organic phases are combined and passed through a phase separator cartridge to give the title compound.

### Step II: 2-(4-bromo-3-methyl-phenyl)-5-methyl-1,3,4-oxadiazole

The title compound is prepared according to the procedure described in: Org. Biomol. Chem., 2012, 10, 988.

### Step III, IV: Compound 125

The title compound is prepared from INTERMEDIATE K and 5-(4-bromophenyl)-1-methyl-1H-pyrazole according to the procedure described for COMPOUND 117.

¹H NMR (400 MHz, CD₃OD) δ 7.94 (s, 1H), 7.87 (d, J=8.1 Hz, 1H), 7.57-7.43 (m, 3H), 7.39 (d, J=8.0 Hz, 1H), 7.28 (d, J=7.1 Hz, 1H), 5.00 (d, J=11.4 Hz, 1H), 4.43 (t, J=3.2 Hz, 1H), 3.94-3.79 (m, 2H), 3.74 (t, J=7.7 Hz, 1H), 3.63 (dd, J=7.8, 2.8 Hz, 1H), 3.57-3.46 (m, 1H), 2.62 (s, 3H), 2.35 (s, 3H). LC-MS: m/z=413.22 (M+H+)

### Preparation of Compound 126

### (2R,3S,4R,5S,6R)-2-(hydroxymethyl)-6-[3-[2-methoxy-4-[5-(2-pyridyl)-1,3,4-oxadiazol-2-yl]phenyl]phenyl]tetrahydropyran-3,4,5-triol

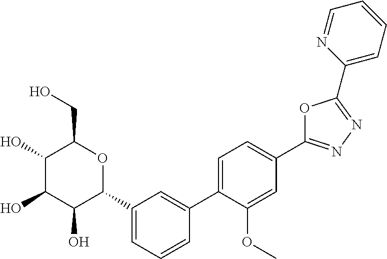 [SMILES: COc1cc(-c2nnc(-c3ccccn3)o2)ccc1-c1cccc([C@H]2O[C@H](CO)[C@@H](O)[C@H](O)[C@@H]2O)c1]

The title compound is prepared from INTERMEDIATE J and 2-(4-bromo-3-methoxyphenyl)-5-(pyridin-2-yl)-1,3,4-oxadiazole according to the procedure described for COMPOUND 117.

¹H NMR (400 MHz, CD₃OD) δ 8.71 (d, J=4.5 Hz, 1H), 8.44 (s, 1H), 8.27 (d, J=7.9 Hz, 1H), 8.08-7.95 (m, J=7.0 Hz, 1H), 7.86-7.74 (m, 2H), 7.66-7.56 (m, 2H), 7.48 (d, J=7.8 Hz, 1H), 7.45-7.29 (m, J=13.5, 8.0 Hz, 3H), 4.96 (d, J=3.2 Hz, 1H), 4.52 (s, 2H), 4.40 (t, J=3.2 Hz, 1H), 3.88 (s, 3H), 3.76 (d, J=4.6 Hz, 2H), 3.68 (t, J=8.1 Hz, 1H), 3.56 (dd, J=8.1, 3.0 Hz, 1H), 3.51-3.43 (m, J=8.3, 4.0 Hz, 1H). LC-MS: m/z=492.41 (M+H+)

### Preparation of Compound 127

### (2R,3S,4R,5S,6R)-2-(hydroxymethyl)-6-[3-[3-methoxy-4-(5-methyl-1,3,4-oxadiazol-2-yl)phenyl]phenyl]tetrahydropyran-3,4,5-triol

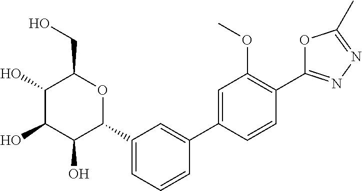 [SMILES: COc1cc(-c2cccc([C@H]3O[C@H](CO)[C@@H](O)[C@H](O)[C@@H]3O)c2)ccc1-c1nnc(C)o1]

The title compound is prepared from INTERMEDIATE J and 2-(4-bromo-2-methoxyphenyl)-5-methyl-1,3,4-oxadiazole according to the procedure described for COMPOUND 117.

¹H NMR (400 MHz, CD₃OD) δ 7.94-7.89 (m, 2H), 7.67-7.63 (m, 1H), 7.50 (d, J=5.9 Hz, 2H), 7.45 (s, 1H), 7.40 (dd, J=8.1, 1.5 Hz, 1H), 5.04 (d, J=3.8 Hz, 1H), 4.51-4.43 (m, 1H), 4.03 (s, 3H), 3.87-3.82 (m, 2H), 3.73 (t, J=7.7 Hz, 1H), 3.66-3.53 (m, 2H), 2.61 (s, 3H). LC-MS: m/z=429.53 (M+H+)

### Preparation of Compound 128

### (2R,3S,4R,5S,6R)-2-(hydroxymethyl)-6-[3-[2-methoxy-4-(5-methyl-1,3,4-oxadiazol-2-yl)phenyl]phenyl]tetrahydropyran-3,4,5-triol

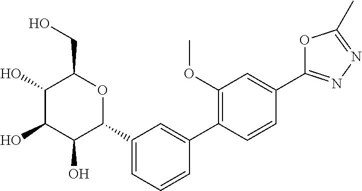 [SMILES: COc1cc(-c2nnc(C)o2)ccc1-c1cccc([C@H]2O[C@H](CO)[C@@H](O)[C@H](O)[C@@H]2O)c1]

The title compound is prepared from INTERMEDIATE J and 2-(4-bromo-3-methoxyphenyl)-5-methyl-1,3,4-oxadiazole according to the procedure described for COMPOUND 117.

¹H NMR (400 MHz, CD₃OD) δ 7.70-7.64 (m, J=7.7 Hz, 3H), 7.52-7.39 (m, 4H), 5.03 (d, J=3.5 Hz, 1H), 4.47 (t, J=3.3 Hz, 1H), 3.90 (s, 3H), 3.84-3.80 (m, J=5.0 Hz, 2H), 3.75 (t, J=8.1 Hz, 1H), 3.62 (dd, J=8.2, 3.0 Hz, 1H), 3.56-3.49 (m, 1H), 3.46 (s, 1H), 2.63 (s, 3H)). LC-MS: m/z=429.44 (M+H+)

### Preparation of Compound 129

### (2R,3S,4R,5S,6R)-2-(hydroxymethyl)-6-[4-[4-(5-methyl-1,3,4-oxadiazol-2-yl)phenyl]phenyl]tetrahydropyran-3,4,5-triol

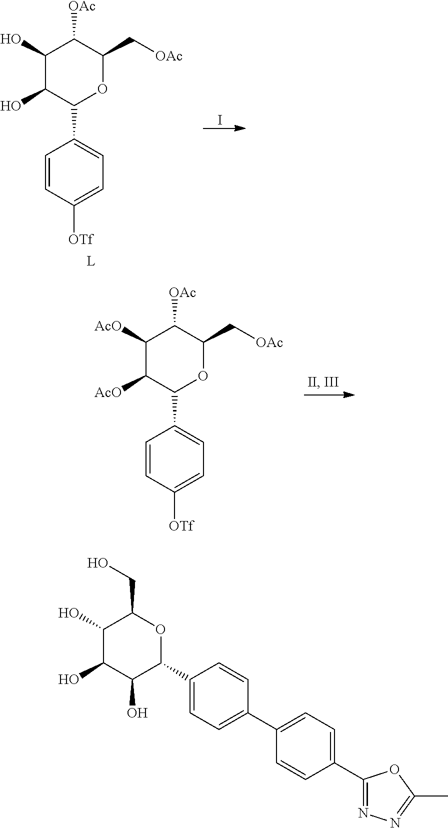

### Step I: (2R,3R,4R,5R,6R)-2-(acetoxymethyl)-6-(4-(((trifluoromethyl)sulfonyl)oxy)phenyl)tetrahydro-2H-pyran-3,4,5-triyltriacetate

The title compound is prepared from INTERMEDIATE L according to the procedure described for INTERMEDIATE H.

### Step II: (2R,3S,4R,5S,6R)-2-(hydroxymethyl)-6-[4-[4-(5-methyl-1,3,4-oxadiazol-2-yl)phenyl]phenyl]tetrahydropyran-3,4,5-triol

The title compound is prepared from (2R,3R,4R,5R,6R)-2-(acetoxymethyl)-6-(4-(((trifluoromethyl)sulfonyl)oxy)phenyl)tetrahydro-2H-pyran-3,4,5-triyl triacetate and [4-(5-methyl-1,3,4-oxadiazol-2-yl)phenyl]boronic acid as starting materials according to the procedure described for COMPOUND 2.

¹H NMR (400 MHz, CD₃OD) δ 8.09 (d, J=8.5 Hz, 2H), 7.84 (d, J=8.6 Hz, 2H), 7.73 (d, J=8.4 Hz, 2H), 7.60 (d, J=8.1 Hz, 2H), 5.02 (d, J=3.7 Hz, 1H), 4.45 (t, J=3.4 Hz, 1H), 3.84 (d, J=5.3 Hz, 2H), 3.75 (t, J=7.9 Hz, 1H), 3.64-3.57 (m, J=8.0, 3.1 Hz, 1H), 3.56-3.41 (m, 1H), 2.62 (s, 3H). LC-MS: m/z=399.41 (M+H+).

### Preparation of Compound 130

### (2R,3S,4R,5S,6R)-2-(hydroxymethyl)-6-[4-[3-(5-methyl-1,3,4-oxadiazol-2-yl)phenyl]phenyl]tetrahydropyran-3,4,5-triol

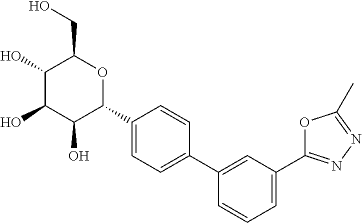 [SMILES: Cc1nnc(-c2cccc(-c3ccc([C@H]4O[C@H](CO)[C@@H](O)[C@H](O)[C@@H]4O)cc3)c2)o1]

The title compound is prepared from INTERMEDIATE J and 2-(3-bromophenyl)-5-methyl-1,3,4-oxadiazole according to the procedure described for COMPOUND 117.

¹H NMR (400 MHz, CD₃OD) δ 8.19 (s, 1H), 7.91 (d, J=7.9 Hz, 1H), 7.78 (d, J=7.9 Hz, 1H), 7.68-7.49 (m, J=14.8, 8.1 Hz, 5H), 4.94 (d, J=3.7 Hz, 1H), 4.52 (s, 1H), 4.38 (t, J=3.4 Hz, 1H), 3.84-3.73 (m, 2H), 3.67 (t, J=7.9 Hz, 1H), 3.53 (dd, J=8.0, 3.1 Hz, 1H), 3.48-3.34 (m, 1H), 2.54 (s, 3H). LC-MS: m/z=399.41 (M+H+)

### Preparation of Compound 131

### (2R,3S,4R,5S,6R)-2-(hydroxymethyl)-6-[2-methyl-3-[2-methyl-4-(5-methyl-1,3,4-oxadiazol-2-yl)phenyl]phenyl]tetrahydropyran-3,4,5-triol

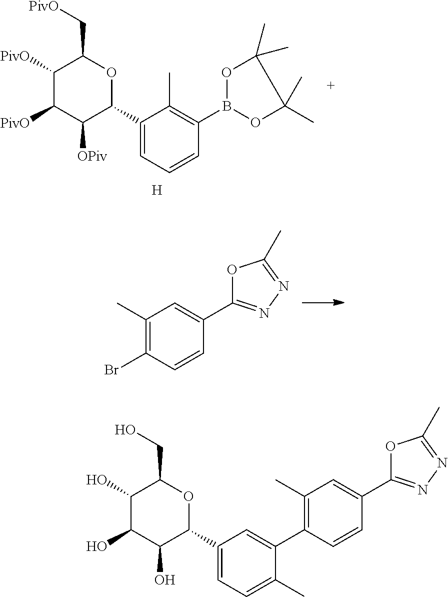

The title compound is prepared from INTERMEDIATE H and 2-(4-bromo-3-methyl-phenyl)-5-methyl-1,3,4-oxadiazole according to the procedure described for COMPOUND 79.

₁H NMR (400 MHz, CD₃OD) δ 7.86 (s, 1H), 7.79 (d, J=8.4 Hz, 1H), 7.46 (d, J=7.7 Hz, 1H), 7.26-7.08 (m, J=14.3, 7.7 Hz, 2H), 6.95 (d, J=7.4 Hz, 1H), 5.10 (d, J=6.9 Hz, 1H), 4.22-4.15 (m, 1H), 3.99-3.87 (m, 2H), 3.75 (q, J=9.7, 4.9 Hz, 1H), 3.66 (dd, J=11.8, 3.7 Hz, 1H), 3.59-3.46 (m, J=9.9 Hz, 1H), 2.54 (s, 3H), 2.06 (s, 3H), 2.03 (s, 3H). LC-MS: m/z=427.45 (M+H+)

### Preparation of Compound 132

### (2R,3S,4R,5S,6R)-2-(hydroxymethyl)-6-[4-methoxy-3-[4-(5-methyl-1,3,4-oxadiazol-2-yl)phenyl]phenyl]tetrahydropyran-3,4,5-triol

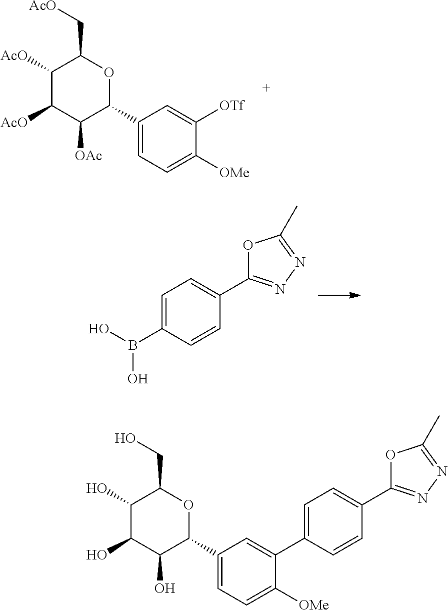 [SMILES: COc1ccc([C@H]2O[C@H](COC(C)=O)[C@@H](OC(C)=O)[C@H](OC(C)=O)[C@@H]2OC(C)=O)cc1OS(=O)(=O)C(F)(F)F ; Cc1nnc(-c2ccc(B(O)O)cc2)o1 ; COc1ccc([C@H]2O[C@H](CO)[C@@H](O)[C@H](O)[C@@H]2O)cc1-c1ccc(-c2nnc(C)o2)cc1]

The title compound is prepared from INTERMEDIATE M and [4-(5-methyl-1,3,4-oxadiazol-2-yl)phenyl]boronic acid according to the procedure described for COMPOUND 2. ¹H NMR (400 MHz, CD₃OD) δ 8.05 (d, J=8.0 Hz, 2H), 7.74 (d, J=8.1 Hz, 2H), 7.57-7.43 (m, 2H), 7.13 (d, J=8.4 Hz, 1H), 4.98 (s, 1H), 4.62 (s, 1H), 4.43 (s, 1H), 3.84 (s, 4H), 3.79-3.70 (m, 1H), 3.70-3.63 (m, 1H), 3.57-3.50 (m, 1H), 2.64 (s, 3H). LC-MS: m/z=429.53 (M+H+).

### Preparation of Compound 133

### (2R,3S,4R,5S,6R)-2-(hydroxymethyl)-6-[3-[2-[4-(5-methyl-1,3,4-oxadiazol-2-yl)phenyl]ethynyl]phenyl]tetrahydropyran-3,4,5-triol

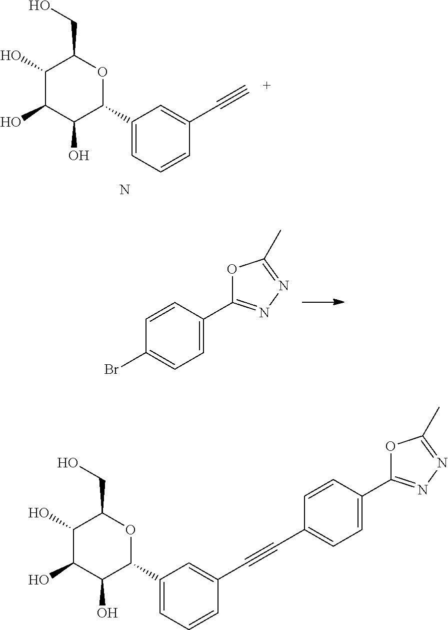

To a degassed (house vacuum/nitrogen flush) mixture of INTERMEDIATE N (27 mg, 0.101 mmol) and 2-(4-bromophenyl)-5-methyl-1,3,4-oxadiazole (28.88 mg, 0.121 mmol), Copper Iodide (4.19 mg, 0.022 mmol) in DMF (2.200 mL) is added d PdCl₂(dppf)2-CH₂Cl₂ (18.44 mg, 0.0226 mmol) and triethylamine (61 mg, 84 μL, 0.604 mmol) heated at 95° C. overnight, cooled to room temperature, diluted with water, extracted with ethyl acetate, combined extracts are washed with brine, dried (Na₂SO₄), concentrated, purified on reverse phase HPLC to afford the title compound.

¹H NMR (400 MHz, CD₃OD) δ 7.95 (d, J=8.4 Hz, 2H), 7.68-7.56 (m, J=8.2 Hz, 3H), 7.53-7.24 (m, 3H), 4.86 (d, J=4.1 Hz, 1H), 4.33-4.25 (m, 1H), 3.83-3.64 (m, 2H), 3.58-3.52 (m, J=7.5, 3.0 Hz, 1H), 3.47-3.34 (m, J=21.1, 10.4, 5.4 Hz, 2H), 2.54 (s, 3H). LC-MS: m/z=423.39 (M+H+).

### Preparation of Compound 134

### (2R,3S,4R,5S,6R)-2-(hydroxymethyl)-6-[4-[2-[4-(5-methyl-1,3,4-oxadiazol-2-yl)phenyl]ethynyl]phenyl]tetrahydropyran-3,4,5-triol

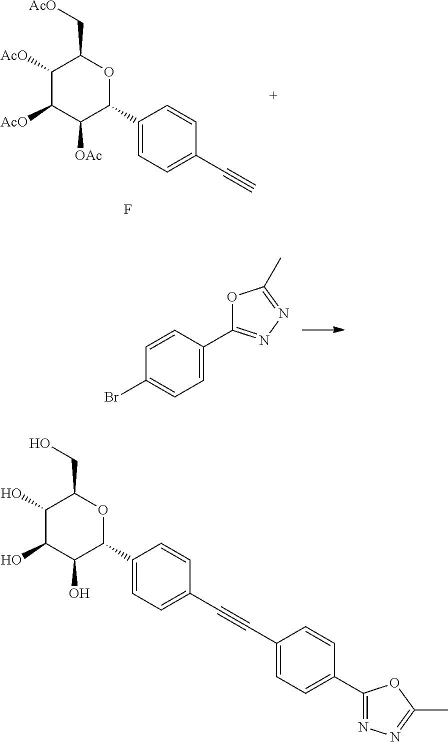

The title compound is prepared using INTERMEDIATE F and 2-(4-bromophenyl)-5-methyl-1,3,4-oxadiazole according to the procedure described for COMPOUND 133

¹H NMR (400 MHz, CD₃OD) δ 7.95 (d, J=8.2 Hz, 2H), 7.61 (d, J=8.1 Hz, 2H), 7.46 (dd, J=14.8, 8.1 Hz, 4H), 4.91-4.87 (m, 1H), 4.30 (t, 1H), 3.81-3.71 (m, 2H), 3.70-3.62 (m, 1H), 3.55-3.47 (m, J=5.0 Hz, 2H), 3.44-3.41 (m, 1H), 2.54 (s, 3H). LC-MS: m/z=423.40 (M+H+)

### Preparation of Compound 135 and 136

### (N1,N3-dimethyl-5-[(Z)-2-[3-[(2R,3S,4R,5S,6R)-3,4,5-trihydroxy-6-(hydroxymethyl)tetrahydropyran-2-yl]phenyl]vinyl]benzene-1,3-dicarboxamide (135)

### (N1,N3-dimethyl-5-[(E)-2-[3-[(2R,3S,4R,5S,6R)-3,4,5-trihydroxy-6-(hydroxymethyl)tetrahydropyran-2-yl]phenyl]vinyl]benzene-1,3-dicarboxamide (136)

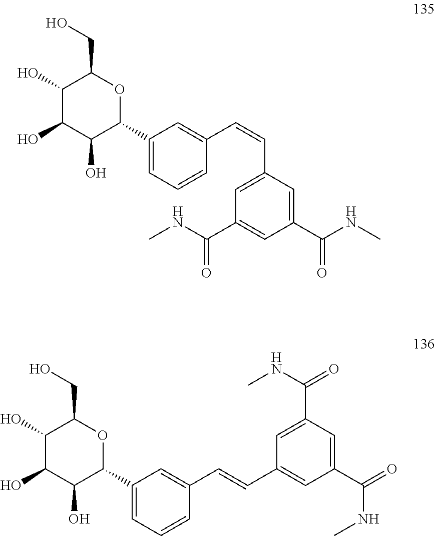 [SMILES: CNC(=O)c1cc(/C=C\c2cccc([C@H]3O[C@H](CO)[C@@H](O)[C@H](O)[C@@H]3O)c2)cc(C(=O)NC)c1 ; CNC(=O)c1cc(/C=C/c2cccc([C@H]3O[C@H](CO)[C@@H](O)[C@H](O)[C@@H]3O)c2)cc(C(=O)NC)c1]

Under air, a reaction tube is charged with COMPOUND 85 (140.1 mg, 0.2250 mmol), triethylsilane (52.32 mg, 71.87 μL, 0.4500 mmol), Pd(dppf)Cl₂-DCM (2.756 mg, 0.003375 mmol), dppf (6.146 mg, 0.01125 mmol) and copper(+2) cation (Sulfate Ion (1)) (5.387 mg, 0.03375 mmol) in toluene (1.125 mL), water (112.5 μL). The mixture is refluxed for 24 h. The solvents are evaporated under reduced pressure and to the residue is added MeOH (2 mL), Sodium methoxide (225.0 μL of 0.5 M, 0.1125 mmol). After stirring overnight, the reaction mixture is passed through SCX-2 cartridge and rinsed with MeOH. The solvent is removed under reduced pressure and the residue is purified by reverse phase HPLC to afford the two title compounds.

Compound 135: ¹H NMR (400 MHz, CD₃OD) δ 8.08 (s, 1H), 7.97 (t, 1H), 7.71 (d, J=1.3 Hz, 1H), 7.38-7.24 (m, J=15.3, 7.7 Hz, 2H), 7.20-7.16 (m, 1H), 7.12 (d, J=7.6 Hz, 1H), 6.82 (d, J=12.0 Hz, 1H), 6.73 (d, J=12.1 Hz, 1H), 4.82 (d, J=3.3 Hz, 1H), 4.16 (t, J=3.3 Hz, 1H), 3.74-3.58 (m, 3H), 3.48-3.45 (m, 1H), 3.24-3.16 (m, 1H), 3.11 (t, 1H), 2.85 (s, 3H). LC-MS: m/z=457.49 (M+H+)

Compound 136: ¹H NMR (400 MHz, CD₃OD) δ 8.07-8.01 (m, 3H), 7.68 (s, 1H), 7.46-7.39 (m, 1H), 7.36-7.18 (m, 4H), 4.91 (d, J=3.7 Hz, 1H), 4.37 (t, J=3.4 Hz, 1H), 3.77 (d, J=4.9 Hz, 2H), 3.66 (t, J=7.8 Hz, 1H), 3.52 (dd, J=8.0, 2.9 Hz, 1H), 3.48-3.41 (m, 1H), 3.40-3.38 (m, 2H), 2.86 (s, 6H). LC-MS: m/z=457.41 (M+H+)

### Preparation of Compound 137 and 138

### (2R,3S,4R,5S,6R)-2-[3-[(Z)-2-(3,5-dichlorophenyl)vinyl]phenyl]-6-(hydroxymethyl)tetrahydropyran-3,4,5-triol (137)

### (2R,3S,4R,5S,6R)-2-[3-[(E)-2-(3,5-dichlorophenyl)vinyl]phenyl]-6-(hydroxymethyl)tetrahydropyran-3,4,5-triol (138)

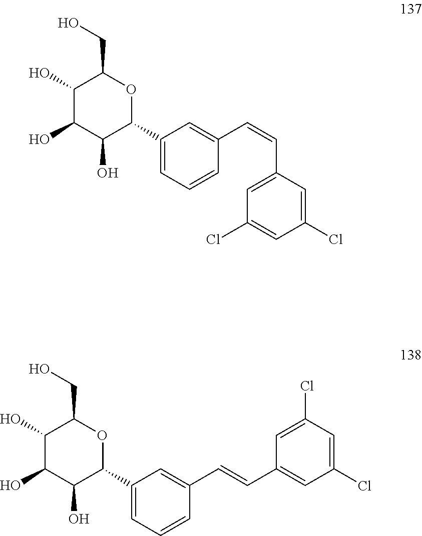 [SMILES: OC[C@H]1O[C@H](c2cccc(/C=C\c3cc(Cl)cc(Cl)c3)c2)[C@@H](O)[C@@H](O)[C@@H]1O ; OC[C@H]1O[C@H](c2cccc(/C=C/c3cc(Cl)cc(Cl)c3)c2)[C@@H](O)[C@@H](O)[C@@H]1O]

The title compounds are prepared from COMPOUND 92 according to the procedure described for COMPOUND 135 and 136.

Compound 137: ¹H NMR (400 MHz, CD₃OD) δ 7.33-7.14 (m, 4H), 7.08-7.01 (m, J=1.6 Hz, 3H), 6.70 (d, J=12.1 Hz, 1H), 6.48 (d, J=12.1 Hz, 1H), 4.53 (s, 1H), 4.24 (t, J=3.3 Hz, 1H), 3.76-3.58 (m, 3H), 3.45 (dd, J=8.1, 3.1 Hz, 2H). LC-MS: m/z=412.28 (M+H+)

Compound 138: ¹H NMR (400 MHz, CD₃OD) δ 7.65 (s, 1H), 7.45 (d, J=1.8 Hz, 2H), 7.42 (d, J=6.8 Hz, 1H), 7.34-7.27 (m, 2H), 7.24-7.18 (m, 2H), 7.08 (s, 1H), 7.04 (s, 1H), 4.89 (d, J=3.7 Hz, 1H), 4.36 (t, J=3.5 Hz, 1H), 3.76 (d, J=4.9 Hz, 2H), 3.65 (t, J=7.8 Hz, 1H), 3.51 (dd, J=7.9, 3.1 Hz, 1H), 3.48-3.38 (m, 1H). LC-MS: m/z=412.28 (M+H+)

### Preparation of Compound 139 and 140

### (2R,3S,4R,5S,6R)-2-(hydroxymethyl)-6-[3-[(Z)-styryl]phenyl]tetrahydropyran-3,4,5-triol (139)

### (2R,3S,4R,5S,6R)-2-(hydroxymethyl)-6-[3-[(E)-styryl]phenyl]tetrahydropyran-3,4,5-triol (140)

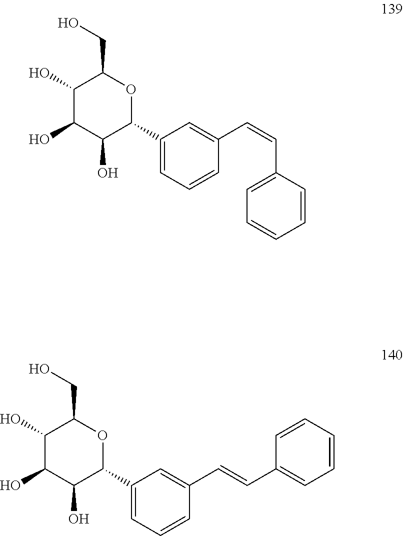 [SMILES: OC[C@H]1O[C@H](c2cccc(/C=C\c3ccccc3)c2)[C@@H](O)[C@@H](O)[C@@H]1O ; OC[C@H]1O[C@H](c2cccc(/C=C/c3ccccc3)c2)[C@@H](O)[C@@H](O)[C@@H]1O]

The title compounds are prepared from COMPOUND 91 according to the procedure described for COMPOUND 135 and 136.

Compound 139: ¹H NMR (400 MHz, CD₃OD) δ 7.35-7.27 (m, J=5.1 Hz, 2H), 7.28-7.09 (m, 7H), 6.62 (d, 2H), 4.27 (t, J=3.2 Hz, 1H), 3.78-3.62 (m, 4H), 3.48 (dd, J=8.3, 3.1 Hz, 2H), 3.39-3.33 (m, 2H). LC-MS: m/z=343.39 (M+H+)

Compound 140: ¹H NMR (400 MHz, CD₃OD) δ 7.70 (s, 1H), 7.54 (d, J=7.3 Hz, 2H), 7.50-7.41 (m, J=3.7 Hz, 1H), 7.40-7.27 (m, 4H), 7.26-7.14 (m, 3H), 4.98 (d, J=3.7 Hz, 1H), 4.46 (t, J=3.4 Hz, 1H), 3.84 (d, J=4.9 Hz, 2H), 3.73 (t, J=7.9 Hz, 1H), 3.61 (dd, J=8.0, 3.1 Hz, 1H), 3.56-3.48 (m, J=7.8, 4.9 Hz, 1H). LC-MS: m/z=343.29 (M+H+)

### Preparation of Compound 141

### N-methyl-2-(1,2,4-triazol-1-yl)-4-[3-[(2R,3S,4R,5S,6R)-3,4,5-trihydroxy-6-(hydroxymethyl)tetrahydropyran-2-yl]phenyl]benzamide

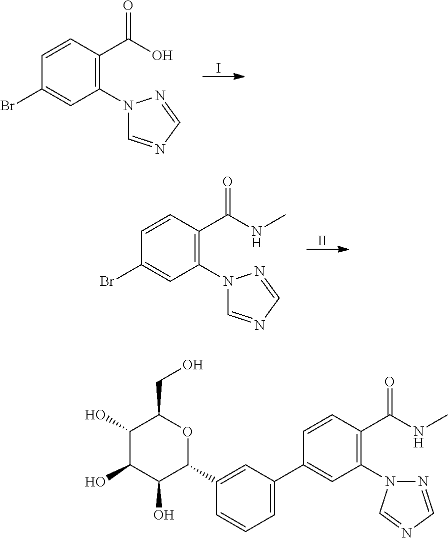

### Step I: 4-bromo-N-methyl-2-(1,2,4-triazol-1-yl)benzamide

The title intermediate is prepared amide coupling of commercially available 4-bromo-2-(1,2,4-triazol-1-yl)benzoic acid and methylamine (solution in ethanol).

### Step II: Compound 141

The title compound is prepared from INTERMEDIATE J and 4-bromo-N-methyl-2-(1,2,4-triazol-1-yl)benzamide (Step 1) according to the procedure described for COMPOUND 117.

¹H NMR (400 MHz, CD₃OD) δ 8.74 (s, 1H), 8.44 (s, 1H), 8.06 (s, 1H), 7.86-7.75 (m, J=10.9 Hz, 2H), 7.66-7.54 (m, 2H), 7.51-7.35 (m, 2H), 4.93 (d, J=4.1 Hz, 1H), 4.52 (s, 1H), 4.35 (t, 1H), 3.82-3.70 (m, J=14.7, 11.8 Hz, 2H), 3.69-3.60 (m, 1H), 3.60-3.51 (m, 1H), 3.52-3.43 (m, 1H), 2.71 (s, 1H). LC-MS: m/z=441.52 (M+H+).

### Preparation of Compound 142

### (2R,3S,4R,5S,6R)-2-(hydroxymethyl)-6-[3-[3-(methoxymethyl)-4-(5-methyl-1,3,4-oxadiazol-2-yl)phenyl]phenyl]tetrahydropyran-3,4,5-triol

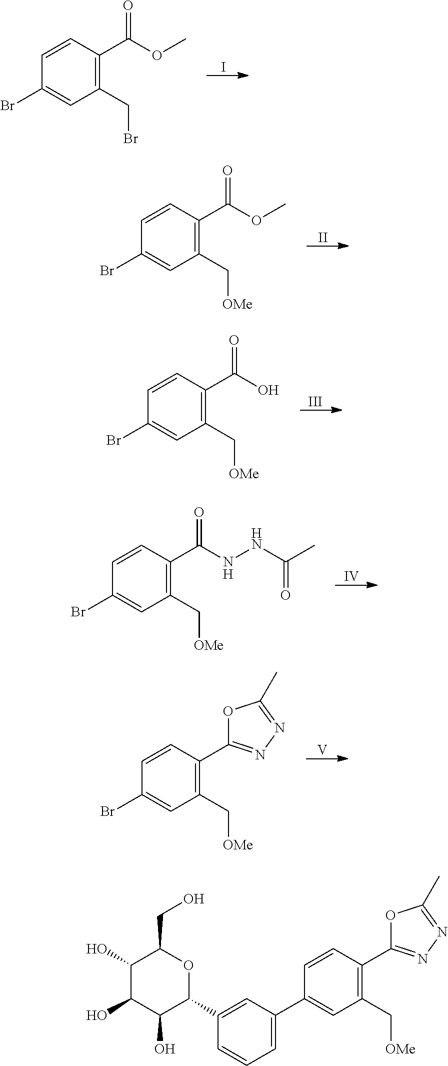

### Step I: Methyl 4-bromo-2-(methoxymethyl)benzoate

To a stirred solution of methyl 4-bromo-2-(bromomethyl)benzoate (1 g, 3.25 mmol) at room temperature in THF (11 mL) is added sodium methoxide (748.7 mg, 772 μL of 25% w/v, 3.57 mmol) and the resulting solution is stirred overnight at 23° C. CH₂Cl₂ (15 ml) and water (5 ml) are added, the phases are separated and the organic phase is passed through a phase separator cartridge. The solvent is removed to afford the title compound.

### Step II: 4-Bromo-2-(methoxymethyl)benzoic acid

To a stirred solution of methyl 4-bromo-2-(methoxymethyl)benzoate (841 mg, 3.25 mmol) in MeOH (11 mL) at room temperature is added NaOH (11 mL of 3 M, 32.46 mmol) and the resulting solution is stirred overnight. The pH is adjust to 7, EtOAc, and water are added. The aqueous phase is extracted. The latter is discarded and the organic phase is passed through a phase separator cartridge. The solvent is removed to afford the title compound.

### Step III: N′-acetyl-4-bromo-2-(methoxymethyl)benzohydrazide

The title compound is prepared according to the procedure described in: Org. Biomol. Chem., 2012, 10, 988.

### Step IV: 2-[4-bromo-2-(methoxymethyl)phenyl]-5-methyl-1,3,4-oxadiazole

The title is prepared according to the procedure described in: Org. Biomol. Chem., 2012, 10, 988.

### Step V: Compound 142

The title compound is prepared from INTERMEDIATE J and 2-[4-bromo-2-(methoxymethyl)phenyl]-5-methyl-1,3,4-oxadiazole (Step 1V) according to the procedure described for COMPOUND 117.

¹H NMR (400 MHz, CD₃OD) δ 7.94 (d, J=8.1 Hz, 1H), 7.88 (s, 1H), 7.79 (s, 1H), 7.69 (d, J=8.3 Hz, 1H), 7.56 (d, J=6.4 Hz, 1H), 7.47-7.37 (m, 2H), 4.95 (d, J=4.0 Hz, 1H), 4.84 (s, 2H), 4.37 (t, 1H), 3.84-3.71 (m, 2H), 3.67 (t, J=7.7 Hz, 1H), 3.56 (dd, J=7.5, 2.9 Hz, 1H), 3.51-3.44 (m, 1H), 3.39 (s, 3H), 2.55 (s, 3H). LC-MS: m/z=443.55 (M+H+)

### Preparation of Compound 143

### (2R,3S,4R,5S,6R)-2-(hydroxymethyl)-6-[3-[3-hydroxy-4-(5-methyl-1,3,4-oxadiazol-2-yl)phenyl]phenyl]tetrahydropyran-3,4,5-triol

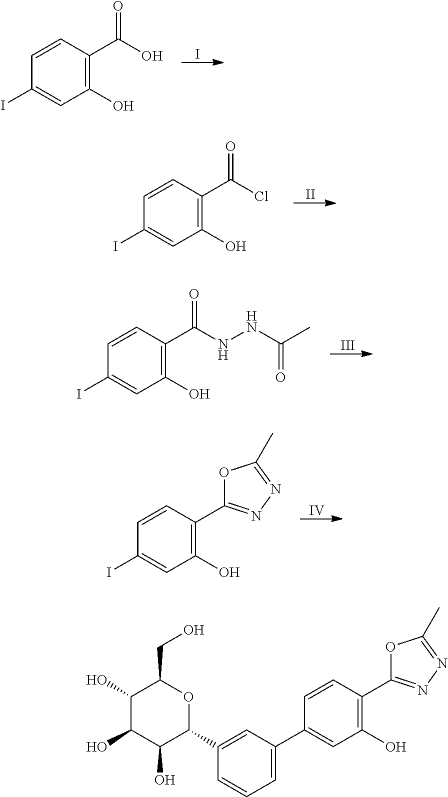

### Step I: 2-hydroxy-4-iodo-benzoyl chloride

Under nitrogen atmosphere, 2-hydroxy-4-iodo-benzoic acid (975 mg, 3.69 mmol) is dissolved in toluene and oxalyl dichloride (3.693 mL of 2 M, 7.39 mmol) is added. The reaction is stirred for 2 minutes and dimethylformamide (13.5 mg, 15 μL, 0.185 mmol) is added. The reaction is stirred at room temperature for 1 hour. The solvent is removed under reduced pressure to afford the title compound. The latter is used as is for the next step.

### Step II: N′-acetyl-2-hydroxy-4-iodo-benzohydrazide

Under nitrogen atmosphere, 2-hydroxy-4-iodo-benzoyl chloride (1.43 g, 5.06 mmol) and acetohydrazide (488 mg, 6.58 mmol) are dissolved in CH₂Cl₂ (17 mL) and triethylamine (1.127 g, 1.55 mL, 11.1 mmol) is added. The reaction is stirred at room temperature overnight. EtOAc and water are added and the phases are separated. Aqueous phase is back extracted one time with EtOAc. The organic phases are combined and passed through a phase separator cartridge. The residue is loaded on a snap 10 g cartridge and eluted O— to 100% of 20% MeOH in CH₂Cl₂ to give the title compound.

### Step III: 5-iodo-2-(5-methyl-1,3,4-oxadiazol-2-yl)phenol

The title compound is prepared according to the procedure described in: Org. Biomol. Chem., 2012, 10, 988.

### Step IV: Compound 143

A microwave vial is charged with A microwave vial is charged with INTERMEDIATE J (23.6 mg, 0.0442 mmol), 5-iodo-2-(5-methyl-1,3,4-oxadiazol-2-yl)phenol (39.33 mg, 0.130 mmol), K₃PO₄ (92.12 mg, 0.434 mmol), Pd(dppf)Cl₂—CH₂Cl₂ (8.86 mg, 0.0109 mmol) in DMF (2.2 mL) and heated in the microwave for 15 minutes at 120° C. The mixture is filtered on Millipore and concentrated to dryness. The residue is dissolved in MeOH (2 mL), treated with NaOMe (25% w/w, 10 μL 0.044 mmol) and stirred overnight at room temperature. The mixture is then filter on SCX-2 SPE column, the column is washed with 2M NH3 in MeOH. The filtrate is concentrated to dryness and purified by reverse phase preparative HPLC to give the title compound.

¹H NMR (400 MHz, CD₃OD) δ 7.84-7.66 (m, J=26.2, 18.6 Hz, 2H), 7.52 (d, J=7.2 Hz, 1H), 7.47-7.33 (m, 2H), 7.33-7.15 (m, J=9.2 Hz, 2H), 4.94 (d, J=3.2 Hz, 1H), 4.37 (t, 1H), 3.86-3.72 (m, 2H), 3.66 (t, J=7.5 Hz, 1H), 3.59-3.42 (m, J=26.2, 10.1 Hz, 2H), 2.56 (s, 3H). LC-MS: m/z=415.34 (M+H+)

### Preparation of Compound 144

### Methyl 2-[3-[(2R,3S,4R,5S,6R)-3,4,5-trihydroxy-6-(hydroxymethyl)tetrahydropyran-2-yl]phenyl]quinoline-6-carboxylate

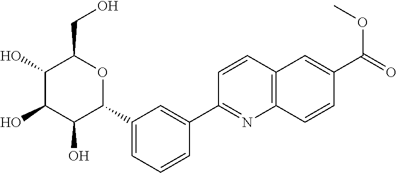 [SMILES: COC(=O)c1ccc2nc(-c3cccc([C@H]4O[C@H](CO)[C@@H](O)[C@H](O)[C@@H]4O)c3)ccc2c1]

The title compound is prepared from INTERMEDIATE J and methyl 2-chloroquinoline-6-carboxylate according to the procedure described for COMPOUND 117.

¹H NMR (400 MHz, CD₃OD) δ 8.60 (d, 1H), 8.50-8.41 (m, 1H), 8.28 (s, 1H), 8.23 (m, 1H), 8.08 (m, 3H), 7.59 (d, 1H), 7.51 (t, 1H), 5.01 (d, 1H), 4.49-4.40 (m, 1H), 3.92 (s, 3H), 3.87-3.75 (m, 2H), 3.71 (t, 1H), 3.61 (m, 1H), 3.53 (m, 1H).

LC-MS: 426.4 (M+H+).

### Preparation of Compound 145

### N-Methyl 2-[3-[(2R,3S,4R,5S,6R)-3,4,5-trihydroxy-6-(hydroxymethyl)tetrahydropyran-2-yl]phenyl]quinoline-6-carboxylate

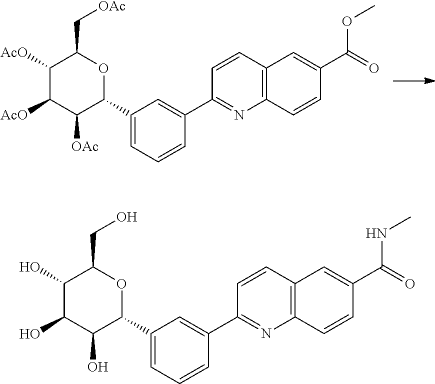 [SMILES: COC(=O)c1ccc2nc(-c3cccc([C@H]4O[C@H](COC(C)=O)[C@@H](OC(C)=O)[C@H](OC(C)=O)[C@@H]4OC(C)=O)c3)ccc2c1 ; CNC(=O)c1ccc2nc(-c3cccc([C@H]4O[C@H](CO)[C@@H](O)[C@H](O)[C@@H]4O)c3)ccc2c1]

A solution of sat. methylamine/EtOH (1 mL) is added to methyl 2-[3-[(2R,3R,4R,5R,6R)-3,4,5-triacetoxy-6-(acetoxymethyl)tetrahydropyran-2-yl]phenyl]quinoline-6-carboxylate (Intermediate in COMPOUND 144 before final deprotection) (15 mg, 0.02527 mmol) in a flask. The reaction mixture is stirred at room temperature overnight. After removal of the volatiles, the residue is purified using reverse-phase prep-HPLC to obtain the title compound (6.6 mg) as a white solid.

¹H NMR (400 MHz, CD₃OD) δ 8.39 (d, 1H), 8.32 (s, 1H), 8.24 (s, 1H), 8.13-7.98 (m, 4H), 7.56 (d, 1H), 7.49 (t, 1H), 4.99 (d, 1H), 4.48-4.31 (m, 1H), 3.89-3.74 (m, 2H), 3.69 (t, 1H), 3.59 (m, 1H), 3.50 (m, 1H), 2.90 (s, 3H). LC-MS: 425.4 (M+H⁺).

### Preparation of Compound 146

### (2R,3S,4R,5S,6R)-2-(Hydroxymethyl)-6-[3-[2-(hydroxymethyl)-1H-benzimidazol-5-yl]phenyl]tetrahydropyran-3,4,5-triol

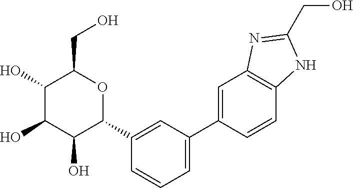 [SMILES: OCc1nc2cc(-c3cccc([C@H]4O[C@H](CO)[C@@H](O)[C@H](O)[C@@H]4O)c3)ccc2[nH]1]

The title compound is prepared from INTERMEDIATE J and (5-bromo-1H-benzo[d]imidazol-2-yl) methanol according to the procedure described for COMPOUND 117.

¹H NMR (400 MHz, CD₃OD) δ 7.73 (s, 1H), 7.70 (d, 1H), 7.49 (m, 3H), 7.42-7.20 (m, 2H), 4.96 (d, 1H), 4.77 (s, 2H), 4.41 (t, 1H), 3.95-3.71 (m, 2H), 3.67 (t, 1H), 3.56 (m, 1H), 3.47 (m, 1H). LC-MS: 387.3 (M+H⁺).

### Preparation of Compound 147

### (2R,3S,4R,5S,6R)-2-[3-(2-Hydroxy-1H-benzimidazol-5-yl)phenyl]-6-(hydroxymethyl)tetrahydropyran-3,4,5-triol

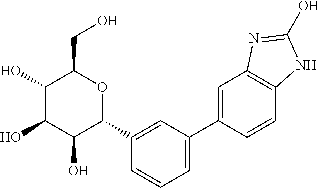 [SMILES: OC[C@H]1O[C@H](c2cccc(-c3ccc4[nH]c(O)nc4c3)c2)[C@@H](O)[C@@H](O)[C@@H]1O]

The title compound is prepared from INTERMEDIATE J and 5-bromo-1H-benzo[d]imidazol-2-ol according to the procedure described for COMPOUND 117.

¹H NMR (400 MHz, CD₃OD) δ 7.74 (s, 1H), 7.50 (d, 1H), 7.46-7.37 (m, 2H), 7.32 (m, 2H), 7.09 (d, 1H), 5.02 (d, 1H), 4.47 (t, 1H), 3.84 (d, 2H), 3.74 (t, 1H), 3.62 (m, 1H), 3.58-3.49 (m, 1H). LC-MS: 373.3 (M+H⁺).

### Preparation of Compound 148

### (2R,3S,4R,5S,6R)-2-(Hydroxymethyl)-6-[3-[2-(trifluoromethyl)-1H-benzimidazol-5-yl]phenyl]tetrahydropyran-3,4,5-triol

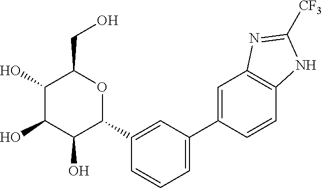 [SMILES: OC[C@H]1O[C@H](c2cccc(-c3ccc4[nH]c(C(F)(F)F)nc4c3)c2)[C@@H](O)[C@@H](O)[C@@H]1O]

The title compound is prepared from INTERMEDIATE J and 5-bromo-2-(trifluoromethyl)-1H-benzo[d]imidazole according to the procedure described for COMPOUND 117.

¹H NMR (400 MHz, CD₃OD) δ 7.84 (s, 1H), 7.73 (t, 2H), 7.65-7.37 (m, 4H), 5.04 (d, 1H), 4.48 (t, 1H), 3.85 (m, 2H), 3.74 (m, 1H), 3.64 (m, 1H), 3.60-3.48 (m, 1H). LC-MS: 425.3 (M+H⁺).

### Preparation of Compound 149

### (2R,3S,4R,5S,6R)-2-[3-(3-Butylbenzimidazol-5-yl)phenyl]-6-(hydroxymethyl)tetrahydropyran-3,4,5-triol

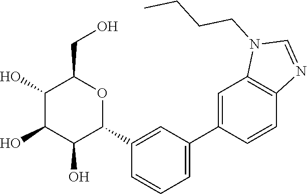 [SMILES: CCCCn1cnc2ccc(-c3cccc([C@H]4O[C@H](CO)[C@@H](O)[C@H](O)[C@@H]4O)c3)cc21]

The title compound is prepared from INTERMEDIATE J and 6-bromo-1-butyl-1H-benzo[d]imidazole according to the procedure described for COMPOUND 117.

¹H NMR (400 MHz, CD₃OD) δ 8.19 (s, 1H), 7.89 (s, 1H), 7.80 (s, 1H), 7.63 (m, 2H), 7.58 (d, 1H), 7.51-7.33 (m, 2H), 5.05 (d, 1H), 4.50 (t, 1H), 4.31 (t, 2H), 3.91-3.80 (m, 2H), 3.75 (t, 1H), 3.65 (m, 1H), 3.60-3.49 (m, 1H), 2.01-1.80 (m, 2H), 1.35 (m, 2H), 0.96 (t, 3H). LC-MS: 413.4 (M+H⁺).

### Preparation of Compound 150

### (2R,3S,4R,5S,6R)-2-[3-[3-Fluoro-4-(5-methyl-1,3,4-oxadiazol-2-yl)phenyl]phenyl]-6-(hydroxymethyl)tetrahydropyran-3,4,5-triol

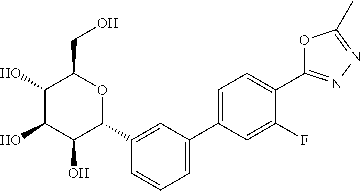 [SMILES: Cc1nnc(-c2ccc(-c3cccc([C@H]4O[C@H](CO)[C@@H](O)[C@H](O)[C@@H]4O)c3)cc2F)o1]

The title compound is prepared from INTERMEDIATE J and 2-(4-bromo-2-fluorophenyl)-5-methyl-1,3,4-oxadiazole according to the procedure described for COMPOUND 117.

¹H NMR (400 MHz, CD₃OD) δ 8.20-8.00 (m, 1H), 7.89 (s, 1H), 7.77-7.60 (m, 3H), 7.51 (m, 2H), 5.02 (d, 1H), 4.50-4.38 (m, 1H), 3.85 (m, 2H), 3.74 (t, 1H), 3.63 (m, 1H), 3.56 (m, 1H), 2.64 (s, 3H). LC-MS: 417.3 (M+H⁺).

### Preparation of Compound 151

### Methyl 5-[3-[(2R,3S,4R,5S,6R)-3,4,5-trihydroxy-6-(hydroxymethyl)tetrahydropyran-2-yl]phenyl]-1H-benzimidazole-2-carboxylate

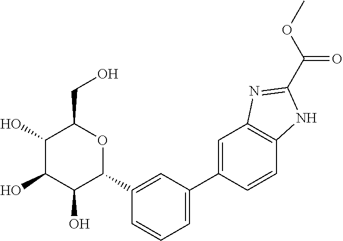 [SMILES: COC(=O)c1nc2cc(-c3cccc([C@H]4O[C@H](CO)[C@@H](O)[C@H](O)[C@@H]4O)c3)ccc2[nH]1]

The title compound is prepared from INTERMEDIATE J and methyl 5-bromo-1H-benzo[d]imidazole-2-carboxylate according to the procedure described for COMPOUND 117.

¹H NMR (400 MHz, CD₃OD) δ 7.92-7.46 (m, 5H), 7.38 (d, 2H), 4.96 (d, 1H), 4.41 (t, 1H), 3.83-3.74 (m, 2H), 3.67 (t, 1H), 3.56 (m, 1H), 3.50-3.44 (m, 1H), 2.91 (s, 3H). LC-MS: 414.4 (M+H⁺).

### Preparation of Compound 152

### N-Methyl-2-[5-[3-[(2R,3S,4R,5S,6R)-3,4,5-trihydroxy-6-(hydroxymethyl)tetrahydropyran-2-yl]phenyl]benzimidazol-1-yl]acetamide

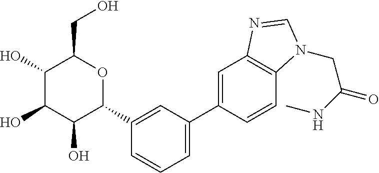 [SMILES: CNC(=O)Cn1cnc2cc(-c3cccc([C@H]4O[C@H](CO)[C@@H](O)[C@H](O)[C@@H]4O)c3)ccc21]

The title compound is prepared from INTERMEDIATE J and 2-(5-bromo-1H-benzo[d]imidazol-1-yl)-N-methylacetamide according to the procedure described for COMPOUND 117.

¹H NMR (400 MHz, CD₃OD) δ 9.48 (s, 1H), 8.07 (d, 1H), 8.01-7.83 (m, 3H), 7.71-7.61 (m, 1H), 7.51 (d, 2H), 5.34 (d, 2H), 5.03 (d, 1H), 4.45 (m, 1H), 3.95-3.80 (m, 2H), 3.78-3.68 (m, 1H), 3.68-3.62 (m, 1H), 3.60-3.53 (m, 1H), 2.82 (d, 3H). LC-MS: 428.6 (M+H⁺).

### Preparation of Compound 153

### 5-[3-[(2R,3S,4R,5S,6R)-3,4,5-trihydroxy-6-(hydroxymethyl)tetrahydropyran-2-yl]phenyl]benzene-1,3-dicarboxylic acid

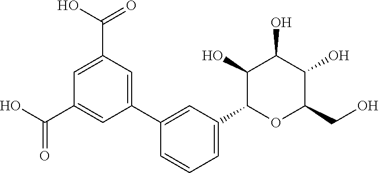 [SMILES: O=C(O)c1cc(C(=O)O)cc(-c2cccc([C@H]3O[C@H](CO)[C@@H](O)[C@H](O)[C@@H]3O)c2)c1]

To a solution of COMPOUND 62 (28 mg, 0.065 mmol) in THF (560 μL) and H₂O (560 μL) is added LiOH (15 mg, 0.36 mmol). After stirring overnight, the reaction mixture is passed through a prewashed (H₂O, MeOH then H₂O)SCX-2 1 g cartridge, and rinsed with H₂O/THF mixture (1:1, 3×1 mL). Combined filtrates concentrated and purified by reverse phase flash chromatography on Biotage C18 snap 12 g cartridge, using a gradient of 0-50% MeCN in H₂O as eluent. The combined fractions are concentrated and freeze-dried to provide title compound as a fluffy white solid (21 mg, 77% yield).

¹H NMR (400 MHz, CD₃OD) δ 8.63 (s, 1H), 8.49 (d, J=1.2 Hz, 2H), 7.85 (s, 1H), 7.63 (d, J=7.1 Hz, 1H), 7.60-7.45 (m, 2H), 5.05 (d, J=4.4 Hz, 1H), 4.45 (dd, J=4.2, 3.3 Hz, 1H), 3.91 (dd, J=11.9, 6.9 Hz, 1H), 3.83 (dd, J=11.9, 3.0 Hz, 1H), 3.78 (t, J=7.3 Hz, 1H), 3.69 (dd, J=7.5, 3.1 Hz, 1H), 3.58 (td, J=6.9, 3.0 Hz, 1H). ESI-MS m/z calc. 404.11072, found 405.33 (M+1)⁺

### Preparation of Compound 154

### N-methyl-3-[6-[(2R,3S,4R,5S,6R)-3,4,5-trihydroxy-6-(hydroxymethyl)tetrahydropyran-2-yl]-2-naphthyl]benzamide

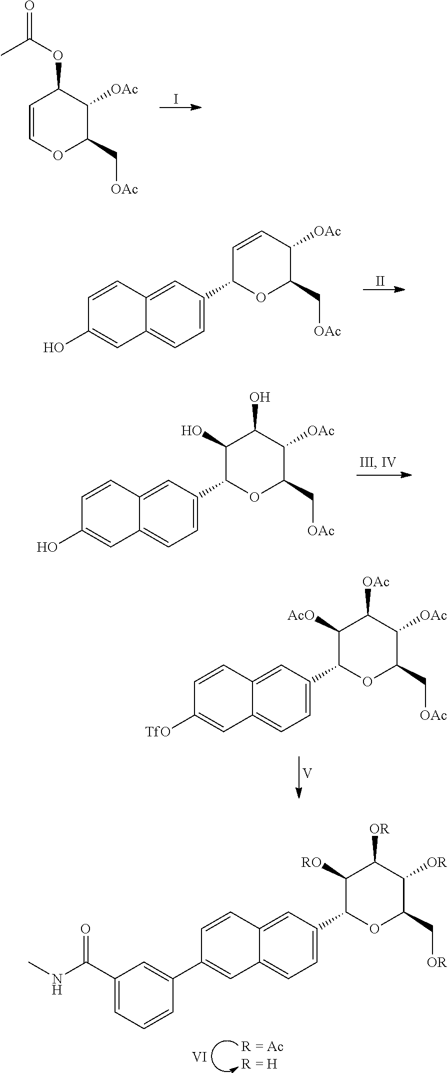

### Step I: [(2R,3S,65)-3-acetoxy-6-(6-hydroxy-2-naphthyl)-3,6-dihydro-2H-pyran-2-yl]methyl acetate

Prepared from (2R,3S,4R)-3,4-diacetoxy-3,4-dihydro-2H-pyran-2-yl]methyl acetate (1.49 g, 5.47 mmol) and (6-hydroxy-2-naphthyl)boronic acid (1.0 g, 5.32 mmol) following the same procedure than INTERMEDIATE A, step 1. Purification by flash chromatography on Biotage™ snap 50 g cartridge using a gradient of EtOAc in hexanes (0-50%) affords the title compound an off-white foamy solid (935 mg, 49% yield).

### Step II: [(2R,3S,4R,5S,6R)-3-acetoxy-4,5-dihydroxy-6-(6-hydroxy-2-naphthyl)tetrahydropyran-2-yl]methyl acetate

Prepared from [(2R,3S,65)-3-acetoxy-6-(6-hydroxy-2-naphthyl)-3,6-dihydro-2H-pyran-2-yl]methyl acetate (930 mg, 2.61 mmol) following the same procedure than INTERMEDIATE A, step II. Purification by flash chromatography on Biotage™ snap 50 g cartridge using a gradient of iPrOH in CH₂Cl₂ (0-10%) affords the title compound as an off-white solid (281 mg, 28% yield).

### Step III: [(2R,3S,4R,5S,6R)-3-acetoxy-4,5-dihydroxy-6-[6-(trifluoromethylsulfonyloxy)-2-naphthyl]tetrahydropyran-2-yl]methyl acetate

To a suspension of [(2R,3S,4R,5S,6R)-3-acetoxy-4,5-dihydroxy-6-(6-hydroxy-2-naphthyl)tetrahydropyran-2-yl]methyl acetate (235 mg, 0.602 mmol) in CH₂Cl₂ (6 mL) is added Et₃N (168 μL, 1.21 mmol) and 1,1,1-trifluoro-N-phenyl-N-(trifluoromethylsulfonyl)methanesulfonamide (266 mg, 0.74 mmol) and another portion of CH₂Cl₂ (4 mL). The resulting suspension is stirred for 3 days, then concentrated and purified on Biotage™ snap 25 g silica cartridge, using a gradient of MeOH in CH₂Cl₂ (0-10%), then purified again Biotage™ snap 25 g silica cartridge, using a gradient of EtOAc in hexanes (50-80%), to afford title compound (214 mg, 68% yield) as a yellowish waxy solid.

### Step IV: [(2R,3R,4R,5R,6R)-3,4,5-triacetoxy-6-[6-(trifluoromethylsulfonyloxy)-2-naphthyl]tetrahydropyran-2-yl]methyl acetate

To a mixture of [(2R,3S,4R,5S,6R)-3-acetoxy-4,5-dihydroxy-6-[6-(trifluoromethylsulfonyloxy)-2-naphthyl]tetrahydropyran-2-yl]methyl (214 mg, 0.41 mmol) in CH₂Cl₂ (2.2 mL) is added sequentially pyridine (100 μL, 1.24 mmol), DMAP (2.5 mg, 0.02 mmol) and Ac₂O (97 μL, 1.03 mmol). After stirring for 2 h, the reaction mixture is diluted with CH₂Cl₂ (5 mL) and aqueous 1N HCl (5 mL). The layers are separated. The aqueous layer is extracted with CH₂Cl₂ (2×5 mL). The combined organic extracts are concentrated, redissolved in CH₂Cl₂, treated with prewashed Dowex 50WX4-400 resin, filtered and rinsed with portions of CH₂Cl₂. The combined filtrates are concentrated to provide title compound (232 mg, 93% yield) as an off-white foamy solid.

### Step V: [(2R,3R,4R,5R,6R)-3,4,5-triacetoxy-6-[6-[3-(methylcarbamoyl)phenyl]-2-naphthylitetrahydropyran-2-yl]methyl acetate

A microwave vial is charged with [(2R,3R,4R,5R,6R)-3,4,5-triacetoxy-6-[6-(trifluoromethylsulfonyloxy)-2-naphthylitetrahydropyran-2-yl]methyl acetate (21.5 mg, 0.035 mmol), N-methyl-3-(4,4,5,5-tetramethyl-1,3,2-dioxaborolan-2-yl)benzamide (11.3 mg, 0.043 mmol), Cs₂CO₃ (37.1 mg, 0.11 mmol) and SiliaCat DPP-Pd (15.3 mg, 0.26 mmol/g, 0.0040 mmol). MeCN (500 μL) is added, the vial is degassed (vacuum then N₂, 3×), capped and submitted to microwave irradiation for 10 min at 100° C. The reaction mixture is diluted with EtOAc, passed on a 500 mg silica cartridge and eluted with EtOAc. The combined filtrates are concentrated and used directly in step VI.

### Step VI: Compound 154

The crude material obtained from step V is dissolved in MeOH (500 μL) and treated with MeONa solution in MeOH (35 μL of 0.5 M, 0.018 mmol). After stirring overnight, the reaction mixture is passed through a prewashed 1 g SCX-2 cartridge and rinsed with MeOH (3×1 mL). The combined filtrates are concentrated and the crude material is purified by HPLC (Injected on Phenomenex C18 Gemini AXIA Sum 110 A 21.2×75 mm Hold 10 min-10% ACN/H2O+0.01% TFA-To 40% ACN+0.01% TFA in 20 min). Fractions are combined, concentrated and freeze-dried to provide the title compound (8.1 mg, 53% yield over two steps) as a fluffy white solid.

¹H NMR (400 MHz, CD₃OD) δ 8.12 (t, J=1.7 Hz, 1H), 8.07 (s, 1H), 7.94-7.81 (m, 4H), 7.79-7.68 (m, 2H), 7.62 (d, J=8.6 Hz, 1H), 7.49 (t, J=7.8 Hz, 1H), 5.05 (d, J=3.6 Hz, 1H), 4.50 (t, J=3.4 Hz, 1H), 3.82-3.75 (m, 2H), 3.70 (t, J=7.8 Hz, 1H), 3.60 (dd, J=7.9, 3.1 Hz, 1H), 3.50-3.42 (m, 1H), 2.87 (s, 3H). ESI-MS m/z calc. 423.16818, found 424.4 (M+1)⁺

### Preparation of Compound 155

### (2R,3S,4R,5S,6R)-2-[6-(3,5-dichlorophenyl)-2-naphthyl]-6-(hydroxymethyl)tetrahydropyran-3,4,5-triol

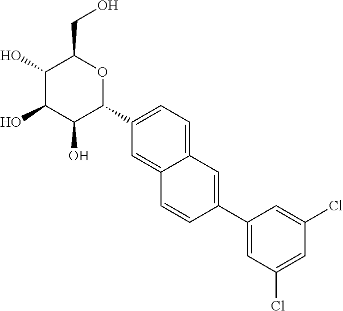 [SMILES: OC[C@H]1O[C@H](c2ccc3cc(-c4cc(Cl)cc(Cl)c4)ccc3c2)[C@@H](O)[C@@H](O)[C@@H]1O]

The title compound is prepared from [(2R,3R,4R,5R,6R)-3,4,5-triacetoxy-6-[6-(trifluoromethylsulfonyloxy)-2-naphthyl]tetrahydropyran-2-yl]methyl acetate (COMPOUND 154, Step 1V) and (3,5-dichlorophenyl)boronic acid following the same procedure of COMPOUND 154 (Step V and VI).

¹H NMR (400 MHz, CD₃OD) δ 8.13 (s, 1H), 8.03-7.94 (m, 3H), 7.78-7.69 (m, 4H), 7.44 (t, J=1.8 Hz, 1H), 5.12 (d, J=3.9 Hz, 1H), 4.60-4.53 (m, 1H), 3.89-3.84 (m, 2H), 3.77 (t, J=7.8 Hz, 1H), 3.67 (dd, J=7.9, 3.1 Hz, 1H), 3.53 (ddd, J=7.4, 6.2, 3.6 Hz, 1H). ESI-MS m/z calc. 435.30, found (M+Na)+457.28, 459.30.

### Preparation of Compound 156

### (2R,3S,4R,5S,6R)-2-(hydroxymethyl)-6-(6-hydroxy-2-naphthyl)tetrahydropyran-3,4,5-triol

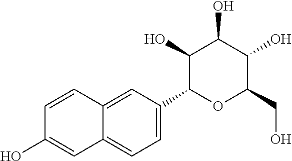 [SMILES: OC[C@H]1O[C@H](c2ccc3cc(O)ccc3c2)[C@@H](O)[C@@H](O)[C@@H]1O]

To a solution of [(2R,3S,4R,5S,6R)-3-acetoxy-4,5-dihydroxy-6-(6-hydroxy-2-naphthyl)tetrahydropyran-2-yl]methyl acetate from Compound 154, Step II (42 mg, 0.107 mmol) in MeOH (1.7 mL) is added MeONa solution in MeOH (54 μL of 0.5 M, 0.027 mmol). After stirring overnight, the resulting suspension is treated with prewashed Dowex 50WX4-400 resin and diluted with THF (2.5 mL), H₂O (0.5 mL) and MeOH (1.5 mL), filtered and washed with portions of THF and MeOH. The combined filtrates are concentrated. Purification by reverse-phase flash chromatography on Biotage™ C18 snap 12 g cartridge, using a gradient of MeCN in H₂O (0-50%) provides title compound (19.6 mg, 59% yield) as an off-white solid.

¹H NMR (400 MHz, CD₃OD δ 9.68 (broad s, 1H), 7.76-7.68 (m, 2H), 7.64 (d, J=8.6 Hz, 1H), 7.47 (dd, J=8.6, 1.4 Hz, 1H), 7.10-7.01 (m, 2H), 4.86-4.70 (m, 2H), 4.60 (2 broad s, 2H), 4.19 (broad s, 1H), 3.65 (broad s, 2H), 3.60-3.52 (m, 1H), 3.48 (dd, J=6.7, 2.8 Hz, 1H), 3.39 (dd, J=11.2, 5.4 Hz, 1H). ESI-MS m/z calc. 306.11035, found 307.29 (M+1)⁺

### Preparation of Compound 157

### N-methyl-3-[[3-[(2R,3S,4R,5S,6R)-3,4,5-trihydroxy-6-(hydroxymethyl)tetrahydropyran-2-yl]phenyl]methyl]benzamide

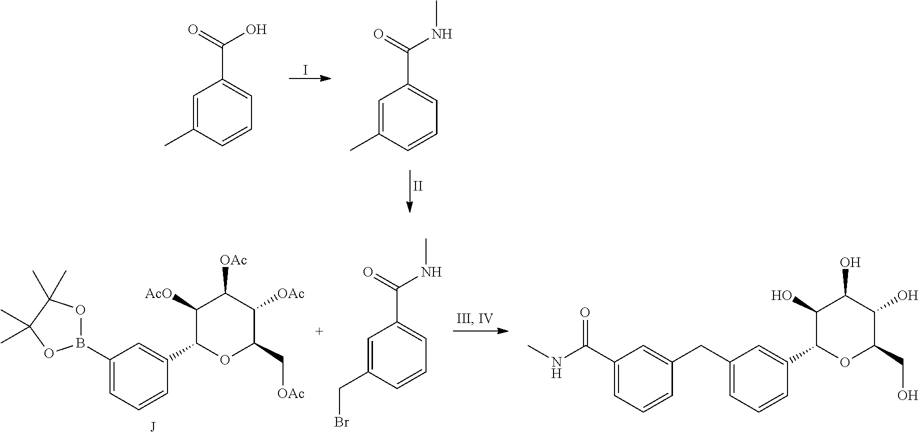

### Step I: N,3-dimethylbenzamide

To a solution of 3-methylbenzoic acid (1.3 g, 9.55 mmol) in DMF (13 mL) is added HATU (4.80 g, 12.6 mmol), followed by DIEA (3.3 mL, 19.1 mmol) then MeNH₂ in THF (7.2 mL of 2 M, 14.3 mmol). After stirring 2 h, the reaction mixture is diluted with EtOAc (100 mL), washed with H₂O (2×50 mL), aqueous 1N HCl (50 ml), brine (50 mL), dried over Na₂SO₄, filtered and concentrated, then purified by flash chromatography on a Biotage™ snap 50 g cartridge, using a gradient of MeOH in CH₂Cl₂ (2-10%). Title compound (1.11 g, 78% yield) is obtained as amber colored oil.

### Step II: 3-(bromomethyl)-N-methyl-benzamide

To a refluxed solution of N,3-dimethylbenzamide (1.1 g, 7.34 mmol) and NBS (1.60 g, 8.99 mmol) in CCl₄ (23 mL) is added AIBN (121 mg, 0.74 mmol). The reaction mixture is stirred at reflux overnight and concentrated, then purified by flash chromatography on a Biotage™ snap 50 g cartridge using a gradient of EtOAc in hexanes (0-70%), affording title compound (761 mg, 45% yield) as a clear yellow oil. ¹H NMR (CDCl₃) shows it contains some succinimide. Used directly for the next step.

### Step III: [(2R,3R,4R,5R,6R)-3,4,5-triacetoxy-6-[3-[[3-(methylcarbamoyl)phenyl]methyl]phenyl]tetrahydropyran-2-yl]methyl acetate

To a solution of INTERMEDIATE J (108 mg, 0.202 mmol) and 3-(bromomethyl)-N-methyl-benzamide (47 mg, 0.206 mmol) THF (1.3 mL) placed in a pressure tube is added aqueous Na₂CO₃ solution (300 μL of 1 M, 0.300 mmol). The reaction mixture is degassed (vacuum then N₂, 3×) and Pd(PPh₃)₄ (7.9 mg, 0.0068 mmol) is added. The reaction mixture is degassed again, capped and heated to 70° C. overnight, then diluted with EtOAc, dried over Na₂SO₄ and passed through an isolute 500 mg silica cartridge, eluting with portions of EtOAc. The combined filtrates are concentrated and the resulting crude product is used directly in the next step.

### Step IV: Compound 157

Crude [(2R,3R,4R,5R,6R)-3,4,5-triacetoxy-6-[3-[[3-(methylcarbamoyl)phenyl]methyl]phenyl]tetrahydropyran-2-yl]methyl acetate from step III is dissolved in MeOH (2.2 mL) and treated with MeONa solution in MeOH (200 μL of 0.5 M, 0.100 mmol) and stirred overnight, then passed on a prewashed SCX-2 1 g cartridge and rinsed with MeOH (3×1 mL). The combined filtrates are concentrated and purified by prep HPLC (caper). After concentration and freeze-drying, title compound (30 mg, 35% yield over two steps) is obtained as a white fluffy solid.

¹H NMR (400 MHz, CD₃OD) δ 7.68 (s, 1H), 7.62 (dt, J=7.2, 1.6 Hz, 1H), 7.43-7.33 (m, 3H), 7.30 (d, J=5.1 Hz, 2H), 7.19-7.11 (m, 1H), 4.95 (d, J=3.5 Hz, 1H), 4.41 (t, J=3.3 Hz, 1H), 4.04 (s, 2H), 3.84-3.75 (m, 2H), 3.72 (t, J=8.1 Hz, 1H), 3.55 (dd, J=8.1, 3.1 Hz, 1H), 3.43 (ddd, J=8.4, 6.0, 3.5 Hz, 1H), 2.90 (s, 3H). ESI-MS m/z calc. 387.43, found 388.39 (M+H)⁺.

### Preparation of Compound 158

### N-methyl-3-[3-methyl-4-[4-[(2R,3S,4R,5S,6R)-3,4,5-trihydroxy-6-(hydroxymethyl)tetrahydropyran-2-yl]phenyl]phenyl]benzamide

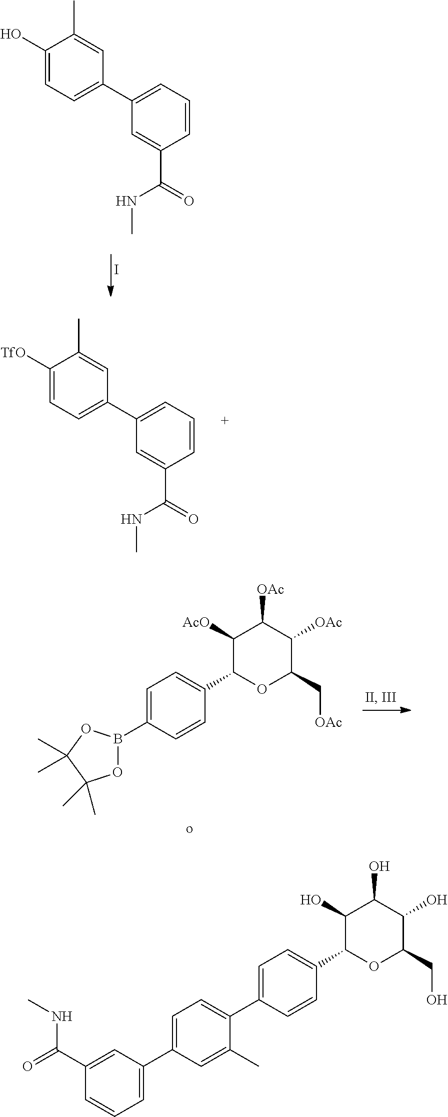

### Step I: [2-methyl-4-[3-(methylcarbamoyl)phenyl]phenyl]trifluoromethanesulfonate

To a solution of 3-(4-hydroxy-3-methyl-phenyl)-N-methyl-benzamide (500 mg, 2.07 mmol) in CH₂Cl₂ (12.5 mL) is added 1,1,1-trifluoro-N-phenyl-N-(trifluoromethylsulfonyl)methanesulfonamide (923 mg, 2.58 mmol) and Et₃N (580 μL, 4.16 mmol). After stirring overnight at room temperature, the reaction mixture is concentrated to dryness then purified by flash chromatography on a Biotage™ snap 25 g cartridge, using a gradient of EtOAc in CH₂Cl₂ (0-50%). Title compound (827 mg, 98% yield) is obtained as a white waxy solid.

### Step II: [(2R,3R,4R,5R,6R)-3,4,5-triacetoxy-6-[4-[2-methyl-4-[3-(methylcarbamoyl)phenyl]phenyl]phenyl]tetrahydropyran-2-yl]methyl acetate

A microwave vial is charged with [2-methyl-4-[3-(methylcarbamoyl)phenyl]phenyl]trifluoromethanesulfonate (79 mg, 0.194 mmol), [(2R,3R,4R,5R,6R)-3,4,5-triacetoxy-6-[4-(4,4,5,5-tetramethyl-1,3,2-dioxaborolan-2-yl)phenyl]tetrahydropyran-2-yl]methyl acetate (compound x, described previously in the patent) (90 mg, 0.87 mmol), Cs₂CO₃ (180 mg, 0.55 mmol) and SiliaCat DPP-Pd (79 mg, 0.26 mmol/g, 0.021 mmol). MeCN (2 mL) is added and the vial is capped and submitted to microwave irradiation for 10 min at 100° C. The reaction mixture is diluted with EtOAc-CH₂Cl₂ mixture (1:1) and passed through a 500 mg silica cartridge and eluted with portions of EtOAc-CH₂Cl₂ (1:1). The combined filtrates are concentrated then purified by flash chromatography on a Biotage™ snap 10 g cartridge, using a gradient of EtOAc in CH₂Cl₂ (0-80%), then purified again by flash chromatography on a Biotage™ snap 10 g cartridge, using a gradient of EtOAc in hexanes (50-80%), affording title compound (30.2 mg, 25% yield).

### Step III: Compound 158

A solution of [(2R,3R,4R,5R,6R)-3,4,5-triacetoxy-6-[4-[2-methyl-4-[3-(methylcarbamoyl)phenyl]phenyl]phenyl]tetrahydropyran-2-yl]methyl acetate (26 mg, 0.041 mmol) in MeOH (1 mL) is treated with MeONa solution in MeOH (42 μL, 0.5M, 0.51 mmol). After stirring overnight, the reaction mixture is passed through a prewashed 1 g SCX-2 cartridge and washed with MeOH (3×1 mL). The combined filtrates are concentrated, suspended in MeCN/H₂O mixture (20% MeCN) and freeze-dried to provide title compound (16.3 mg, 83% yield) as a white fluffy solid.

¹H NMR (400 MHz, CD₃OD) δ 8.12 (t, J=1.6 Hz, 1H), 7.87-7.82 (m, 1H), 7.82-7.77 (m, 1H), 7.66-7.51 (m, 5H), 7.39 (d, J=8.2 Hz, 2H), 7.30 (d, J=7.9 Hz, 1H), 5.06 (d, J=3.6 Hz, 1H), 4.51 (t, J=3.4 Hz, 1H), 3.87 (d, J=4.7 Hz, 2H), 3.78 (t, J=8.0 Hz, 1H), 3.66 (dd, J=8.1, 3.1 Hz, 1H), 3.60-3.51 (m, 1H), 2.96 (s, 3H), 2.35 (s, 3H). ESI-MS m/z calc. 463.52, found 464.51 (M+H)⁺.

### Preparation of Compound 159

### N,3′-dimethyl-3″-((2R,3S,4R,5S,6R)-3,4,5-trihydroxy-6-(hydroxymethyl)tetrahydro-2H-pyran-2-yl)-[1,1′:4′,1″-terphenyl]-3-carboxamide

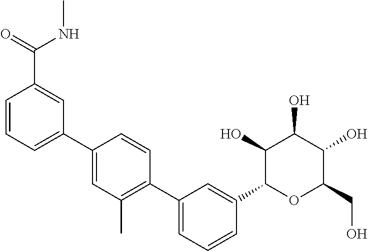 [SMILES: CNC(=O)c1cccc(-c2ccc(-c3cccc([C@H]4O[C@H](CO)[C@@H](O)[C@H](O)[C@@H]4O)c3)c(C)c2)c1]

The title compound is prepared following the procedure described in COMPOUND 158, but using INTERMEDIATE J as the starting material in Step II.

¹H NMR (400 MHz, CD₃OD) δ 8.12 (t, J=1.6 Hz, 1H), 7.87-7.82 (m, 1H), 7.82-7.76 (m, 1H), 7.62 (d, J=1.2 Hz, 1H), 7.58-7.43 (m, 5H), 7.36-7.26 (m, 2H), 5.05 (d, J=3.6 Hz, 1H), 4.49 (t, J=3.4 Hz, 1H), 3.88-3.82 (m, 2H), 3.77 (t, J=7.9 Hz, 1H), 3.66 (dd, J=8.0, 3.1 Hz, 1H), 3.55 (ddd, J=8.0, 5.7, 3.9 Hz, 1H), 2.96 (s, 3H), 2.36 (s, 3H). ESI-MS m/z calc. 463.52, found 464.51 (M+H)⁺.

### Preparation of Compound 160

### (2R,3S,4R,5S,6R)-2-(hydroxymethyl)-6-[4-[6-(5-methyl-1,3,4-oxadiazol-2-yl)-2-naphthyl]phenyl]tetrahydropyran-3,4,5-triol

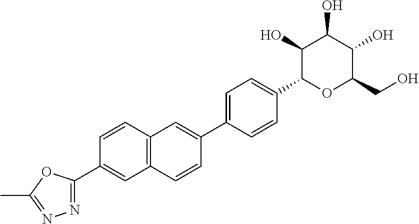 [SMILES: Cc1nnc(-c2ccc3cc(-c4ccc([C@H]5O[C@H](CO)[C@@H](O)[C@H](O)[C@@H]5O)cc4)ccc3c2)o1]

The title compound is prepared following the procedure described in COMPOUND 158 step II and III, using INTERMEDIATE 0 and 2-(6-bromo-2-naphthyl)-5-methyl-1,3,4-oxadiazole as the appropriate starting materials. Step II was conducted on microwave, for a total of 15 minutes at 100° C. The final compound was purified by reverse-phase flash chromatography on a Biotage™ C18 snap 30 g cartridge, using a gradient of MeCN in H₂O (0-80%), then purified again by prep HPLC (caper), affording the title compound (3.8 mg, 5% yield over two steps) as a fluffy white solid.

ESI-MS m/z calc. 448.47, found 449.32 (M+H)⁺.

### Preparation of Compound 161

### (2R,3S,4R,5S,6R)-2-(hydroxymethyl)-6-[3-[6-(5-methyl-1,3,4-oxadiazol-2-yl)-2-naphthyl]phenyl]tetrahydropyran-3,4,5-triol

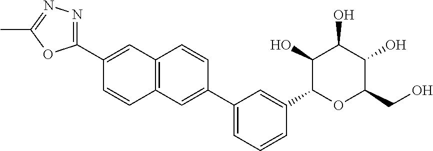 [SMILES: Cc1nnc(-c2ccc3cc(-c4cccc([C@H]5O[C@H](CO)[C@@H](O)[C@H](O)[C@@H]5O)c4)ccc3c2)o1]

The title compound is prepared following the procedure described in COMPOUND 154, Step II and III, using INTERMEDIATE J and 2-(6-bromo-2-naphthyl)-5-methyl-1,3,4-oxadiazole as the appropriate starting materials. Step II was conducted on microwave, for a total of 15 minutes at 100° C. The final compound was purified by reverse-phase flash chromatography on a Biotage™ C18 snap 12 g cartridge, using a gradient of MeCN in H₂O (0-80%), affording the title compound (20.4 mg, 28% yield over two steps) as a fluffy white solid.

¹H NMR (400 MHz, DMSO) δ 8.61 (s, 1H), 8.33 (s, 1H), 8.24 (d, J=8.7 Hz, 1H), 8.18 (d, J=8.7 Hz, 1H), 8.08 (dd, J=8.6, 1.6 Hz, 1H), 7.98 (dd, J=8.6, 1.6 Hz, 1H), 7.92 (s, 1H), 7.75 (d, J=7.4 Hz, 1H), 7.54-7.41 (m, 2H), 4.87-4.76 (m, 3H), 4.70 (dd, J=5.8, 4.5 Hz, 2H), 4.16 (td, J=5.6, 3.2 Hz, 1H), 3.68 (t, J=5.6 Hz, 2H), 3.57 (dd, J=11.8, 5.9 Hz, 1H), 3.52-3.44 (m, 2H), 2.62 (s, 3H). ESI-MS m/z calc. 448.47, found 449.32 (M+H)⁺.

Compound 162 to 171 are prepared according to the general procedure below

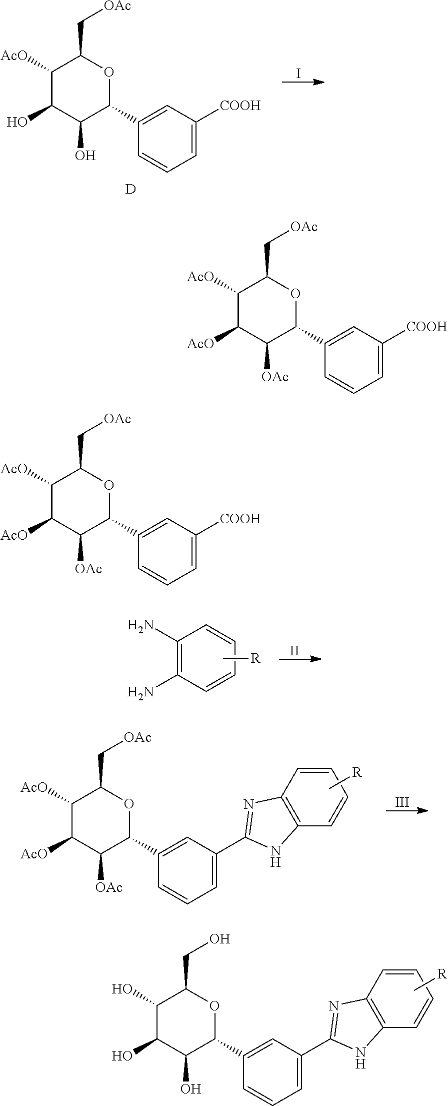

### Step I: 3-((2R,3R,4R,5R,6R)-3,4,5-triacetoxy-6-(acetoxymethyl)tetrahydro-2H-pyran-2-yl)benzoic acid

To a suspension of INTERMEDIATE D (165 mg, 0.551 mmol) in 1.6 mL of CH₂Cl₂ was sequentially added pyridine (312 μL, 3.86 mmol), DMAP (6.7 mg, 0.055 mmol) and Ac₂O (312 μL, 3.31 mmol) under nitrogen atmosphere. The reaction mixture was stirred at room temperature for 20 hours and diluted with 2M HCl (0.5 mL). The organic layer was dried over Na₂SO₄, filtered, and concentrated to dryness. The residue was purified by flash column chromatography on silica gel (0 to 20% MeOH in CH₂Cl₂) to give the raw final product. The product was dissolved in 5 mL of CH₂Cl₂ and washed with 1M HCl (1 mL). The organic layer was dried over Na₂SO₄, filtered, and concentrated to dryness to give the title compound (242.2 mg, 97%).

### Step II

To a solution of 3-[(2R,3R,4R,5R,6R)-3,4,5-triacetoxy-6-(acetoxymethyl)tetrahydropyran-2-yl]benzoic acid (Step I, 1 eq.) and the corresponding orthophenylene diamine (1 eq.) in THF are sequentially added HATU (1 eq.) and DIPEA (1 eq.). The mixture is stirred under nitrogen at room temperature until completion and the reaction mixture is diluted with saturated NaHCO3, extracted by EtOAc and the combined organic layer are washed with H₂O and concentrated to dryness. The residue is dissolved in AcOH and heated to 50° C. overnight. The reaction mixture is then evaporated to dryness and the residue purified by flash column chromatography on silica gel (10 to 80% EtOAc in hexanes) to give the desired material.

### Step III

The COMPOUNDs in Table 4 are deacetylated using previously described procedure.

TABLE 4

| CMPD | | ¹H-NMR | M + 1 |
|---|---|---|---|
| 162 | 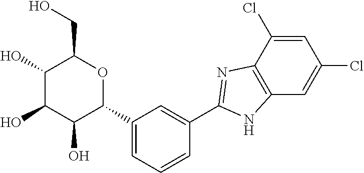 [SMILES: OC[C@H]1O[C@H](c2cccc(-c3nc4c(Cl)cc(Cl)cc4[nH]3)c2)[C@@H](O)[C@@H](O)[C@@H]1O] | (400 MHz, DMSO-d6) δ 13.40 (broad s, 1H), 8.17 (m, 1H), 8.03 (s, 1H), 7.49 (m, 3H), 7.32 (d, J = 1.6 Hz, 1H), 4.83 (broad s, 1H), 4.70 (d, J = 6.5 Hz, 1H), 4.63 (broad s, 1H), 4.54 (broad s, 1H), 3.99 (broad s, 1H), 3.69 (m, 1H), 3.58 (m, 2H), 3.50 (m, 3H). | 425.5 |
| 163 | 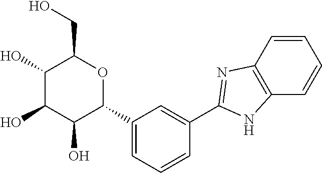 [SMILES: OC[C@H]1O[C@H](c2cccc(-c3nc4ccccc4[nH]3)c2)[C@@H](O)[C@@H](O)[C@@H]1O] | (400 MHz, DMSO-d6) δ 12.87 (s, 1H), 8.05 (m, 2H), 7.46 (m, 4H), 7.13 (m, 2H), 4.81 (broad s, 1H), 4.72 (m, 1H), 4.63 (broad, s, 1H), 4.54 (broad s, 1H), 4.05 (m, 1H), 3.67 (m, 1H), 3.58 (m, 2H), 3.49 (m, 1H), 3.44 (m, 1H), 3.27 (broad s, 1H). | 357.29 |
| 164 | 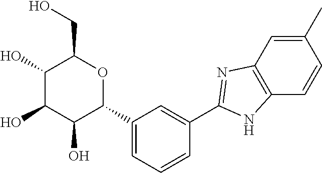 [SMILES: Cc1ccc2[nH]c(-c3cccc([C@H]4O[C@H](CO)[C@@H](O)[C@H](O)[C@@H]4O)c3)nc2c1] | (400 MHz, CD₃OD) δ 8.10 (s, 1H), 7.90 (d, J = 7.7 Hz, 1H), 7.56 (d, J = 7.7 Hz, 1H), 7.47 (t, J = 7.7 Hz, 1H), 7.40 (d, J = 8.3 Hz, 1H), 7.31 (s, 1H), 7.02 (dd, J = 8.3, 1.2 Hz, 1H), 4.96 (d, J = 4.2 Hz, 1H), 4.40 (m, 1H), 3.80 (m, 2H), 3.69 (t, J = 7.5 Hz, 1H), 3.58 (dd, J = 7.7, 3.1 Hz, 1H), 3.47 (m, 1H), 2.38 (s, 3H). | 371.62 |
| 165 | 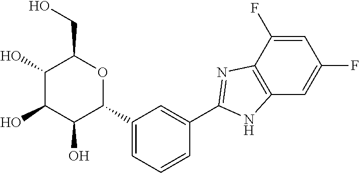 [SMILES: OC[C@H]1O[C@H](c2cccc(-c3nc4c(F)cc(F)cc4[nH]3)c2)[C@@H](O)[C@@H](O)[C@@H]1O] | (400 MHz, CD₃OD) δ 8.13 (s, 1H), 7.94 (d, J = 7.7 Hz, 1H), 7.60 (d, J = 7.8 Hz, 1H), 7.49 (t, J = 7.8 Hz, 1H), 7.06 (s, 1H), 6.81 (t, J = 10.1 Hz, 1H), 4.96 (d, J = 4.2 Hz, 1H), 4.40 (m, 1H), 3.80 (m, 2H), 3.70 (t, J = 7.4 Hz, 1H), 3.59 (m, 1H), 3.48 (m, 1H). | 393.43 |
| 166 | 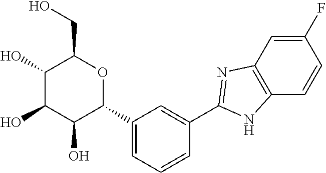 [SMILES: OC[C@H]1O[C@H](c2cccc(-c3nc4cc(F)ccc4[nH]3)c2)[C@@H](O)[C@@H](O)[C@@H]1O] | (400 MHz, CD₃OD) δ 8.18 (s, 1H), 7.99 (d, J = 7.7 Hz, 1H), 7.65 (d, J = 7.8 Hz, 1H), 7.56 (m, 2H), 7.29 (dd, J = 9.1, 2.4 Hz, 1H), 7.04 (td, J = 9.4, 2.4 Hz, 1H), 5.03 (d, J = 4.2 Hz, 1H), 4.47 (m, 1H), 3.89 (m, 2H), 3.78 (m, 1H), 3.66 (dd, J = 7.6, 3.1 Hz, 1H), 3.55 (td, J = 6.8, 3.3 Hz, 1H). | 375.59 |
| 167 | 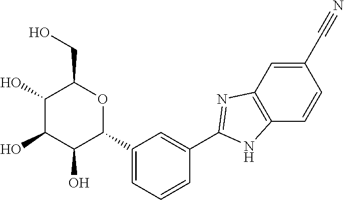 [SMILES: N#Cc1ccc2[nH]c(-c3cccc([C@H]4O[C@H](CO)[C@@H](O)[C@H](O)[C@@H]4O)c3)nc2c1] | (400 MHz, CD₃OD) δ 8.16 (s, 1H), 7.95 (m, 2H), 7.62 (m, 2H), 7.51 (t, J = 7.8 Hz, 2H), 4.96 (d, J = 4.5 Hz, 1H), 4.37 (dd, J = 4.3, 3.3 Hz, 1H), 3.81 (m, 2H), 3.71 (m, 1H), 3.60 (dd, J = 7.4, 3.1 Hz, 1H), 3.49 (m, 1H). | 382.59 |
| 168 | 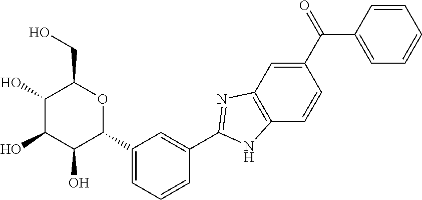 [SMILES: O=C(c1ccccc1)c1ccc2[nH]c(-c3cccc([C@H]4O[C@H](CO)[C@@H](O)[C@H](O)[C@@H]4O)c3)nc2c1] | (400 MHz, CD₃OD) δ 8.17 (s, 1H), 7.99 (s, 1H), 7.71 (m, 3H), 7.68-7.43 (m, 7H), 4.97 (d, J = 4.3 Hz, 1H), 4.40 (m, 1H), 3.81 (m, 2H), 3.72 (m, 1H), 3.60 (dd, J = 7.5, 3.1 Hz, 1H), 3.50 (td, J = 6.9, 3.2 Hz, 1H). | 461.61 |
| 169 | 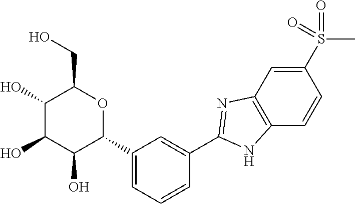 [SMILES: CS(=O)(=O)c1ccc2[nH]c(-c3cccc([C@H]4O[C@H](CO)[C@@H](O)[C@H](O)[C@@H]4O)c3)nc2c1] | (400 MHz, CD₃OD) δ 8.29-8.17 (m, 2H), 8.06 (d, J = 7.8 Hz, 1H) 7.88-7.74 (m, 2H), 7.70 (d, J = 7.9 Hz, 1H), 7.58 (m, 1H), 5.04 (d, J = 4.5 Hz, 1H), 4.45 (dd, J = 4.3, 3.3 Hz, 1H), 3.89 (m, 2H), 3.79 (t, J = 7.2 Hz, 1H), 3.67 (m, 1H), 3.57 (td, J = 6.9, 3.1 Hz, 1H), 3.16 (s, 3H). | 435.58 |
| 170 | 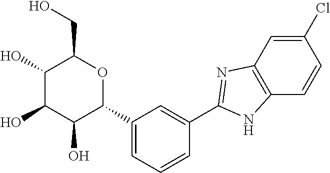 [SMILES: OC[C@H]1O[C@H](c2cccc(-c3nc4cc(Cl)ccc4[nH]3)c2)[C@@H](O)[C@@H](O)[C@@H]1O] | (400 MHz, CD₃OD) δ 8.11 (m, 1H), 7.92 (d, J = 7.7 Hz, 1H), 7.58 (d, J = 7.9 Hz, 1H), 7.50 (m, 3H), 7.17 (dd, J = 8.6, 2.0 Hz, 1H), 4.96 (d, J = 4.3 Hz, 1H), 4.38 (m, 1H), 3.80 (m, 2H), 3.70 (dd, J = 15.0, 7.7 Hz, 1H), 3.58 (dd, J = 7.6, 3.1 Hz, 1H), 3.47 (td, J = 6.8, 3.2 Hz, 1H). | 391.29 |
| 171 | 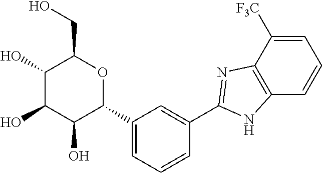 [SMILES: OC[C@H]1O[C@H](c2cccc(-c3nc4c(C(F)(F)F)cccc4[nH]3)c2)[C@@H](O)[C@@H](O)[C@@H]1O] | (400 MHz, CD₃OD) δ 8.24 (s, 1H), 8.05 (d, J = 7.6 Hz, 1H), 7.77 (broad s, 1H), 7.65 (d, J = 7.8 Hz, 1H), 7.54 (m, 2H), 7.34 (t, J = 7.7 Hz, 1H), 5.01 (d, J = 4.4 Hz, 1H), 4.45 (m, 1H), 3.84 (m, 2H), 3.74 (t, J = 7.3 Hz, 1H), 3.64 (dd, J = 7.5, 3.1 Hz, 1H), 3.54 (td, J = 6.9, 3.1 Hz, 1H). | 425.35 |

### Preparation of Compound 172

### N-methyl-2-(3-((2R,3S,4R,5S,6R)-3,4,5-trihydroxy-6-(hydroxymethyl)tetrahydro-2H-pyran-2-yl)phenyl)-1H-benzo[d]imidazole-5-carboxamide

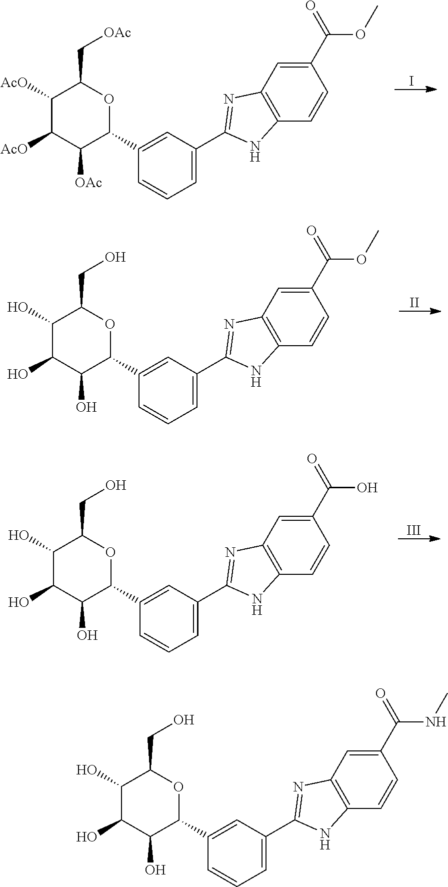

(2R,3R,4R,5R,6R)-2-(acetoxymethyl)-6-(3-(5-(methoxycarbonyl)-1H-benzo[d]imidazol-2-yl)phenyl)tetrahydro-2H-pyran-3,4,5-triyltriacetate is prepared according to the procedure described for COMPOUND 162-171 using methyl 3,4-diaminobenzoate as reagent in Step II.

### Step I: methyl 2-(3-((2R,3S,4R,5S,6R)-3,4,5-trihydroxy-6-(hydroxymethyl)tetrahydro-2H-pyran-2-yl)phenyl)-1H-benzo[d]imidazole-5-carboxylate

To a solution of (2R,3R,4R,5R,6R)-2-(acetoxymethyl)-6-(3-(5-(methoxycarbonyl)-1H-benzo[d]imidazol-2-yl)phenyl)tetrahydro-2H-pyran-3,4,5-triyltriacetate (49.5 mg, 0.0849 mmol) in methanol (2.4 mL) is added NaOMe (0.5 M in MeOH, 42.5 μL, 0.0212 mmol). The mixture is stirred overnight at room temperature, filtered over a 1 g SCX-2 SPE column and concentrated to dryness. The resulting title compound is used as is for next step.

### Step II: 2-(3-((2R,3S,4R,5S,6R)-3,4,5-trihydroxy-6-(hydroxymethyl)tetrahydro-2H-pyran-2-yl)phenyl)-1H-benzo[d]imidazole-5-carboxylic

To a solution of methyl 2-(3-((2R,3S,4R,5S,6R)-3,4,5-trihydroxy-6-(hydroxymethyl)tetrahydro-2H-pyran-2-yl)phenyl)-1H-benzo[d]imidazole-5-carboxylate (0.0849 mmol) in THF (1.5 mL) and water (1.5 mL) is added LiOH (hydrate, 17.8 mg, 0.425 mmol) and the mixture is stirred overnight at room temperature. Another portion of LiOH (hydrate, 17.8 mg, 0.425 mmol) is added and the mixture was heated to 35° C. for 1 hour to complete the reaction. The mixture is then cooled to room temperature, HCl (315 μ of 4 M, 1.274 mmol) is added and the mixture is concentrated to dryness to give the title compound which is used as is for next step.

### Step III: Compound 172

To a solution of 2-(3-((2R,3S,4R,5S,6R)-3,4,5-trihydroxy-6-(hydroxymethyl)tetrahydro-2H-pyran-2-yl)phenyl)-1H-benzo[d]imidazole-5-carboxylic acid (0.0849 mmol) see previous procedure) in DMF (2 mL) is sequentially added MeNH2 (2M in THF, 55.2 μL, 0.110 mmol), HATU (42 mg, 0.110 mmol) and DIPEA (22.2 μL, 0.1274 mmol). The mixture is stirred overnight at room temperature, concentrated to dryness and purified by reverse phase preparative HPLC to the title compound (12.1 mg, 31%).

¹H NMR (400 MHz, CD₃OD) δ 8.50 (m, 1H), 8.23 (s, 1H), 8.11 (m, 1H), 8.04 (m, 1H), 7.76 (m, 1H), 7.68 (m, 2H), 7.58 (t, J=7.8 Hz, 1H), 5.04 (d, J=4.4 Hz, 1H), 4.46 (dd, J=4.3, 3.2 Hz, 1H), 3.88 (m, 2H), 3.78 (t, J=7.3 Hz, 1H), 3.67 (dd, J=7.5, 3.1 Hz, 1H), 3.57 (td, J=6.9, 3.1 Hz, 1H), 2.94 (s, 3H). LC/MS: m/z=414.6 (M+H⁺)

### Preparation of Compound 173

### N-methyl-2-(3-((2R,3S,4R,5S,6R)-3,4,5-trihydroxy-6-(hydroxymethyl)tetrahydro-2H-pyran-2-yl)phenyl)-1H-benzo[d]imidazole-4-carboxamide

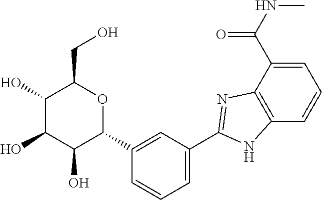 [SMILES: CNC(=O)c1cccc2[nH]c(-c3cccc([C@H]4O[C@H](CO)[C@@H](O)[C@H](O)[C@@H]4O)c3)nc12]

The title compound is prepared according to the procedure described for COMPOUND 163 using methyl 2,3-diaminobenzoate as reagent.

¹H NMR (400 MHz, CD₃OD) δ 8.28 (s, 1H), 8.06 (d, J=7.6 Hz, 1H), 7.85 (d, J=7.3 Hz, 1H), 7.64 (d, J=7.9 Hz, 1H), 7.59 (d, J=7.7 Hz, 1H), 7.51 (t, J=7.7 Hz, 1H), 7.29 (m, 1H), 5.00 (d, J=4.3 Hz, 1H), 4.41 (m, 1H), 3.85 (m, 2H), 3.72 (t, J=7.4 Hz, 1H), 3.60 (m, 1H), 3.54 (td, J=7.0, 3.1 Hz, 1H), 3.06 (d, J=3.5 Hz, 3H). LC/MS: m/z=414.4 (M+H⁺).

Compound 174 to 178 in Table 5 are prepared according to the general procedure below

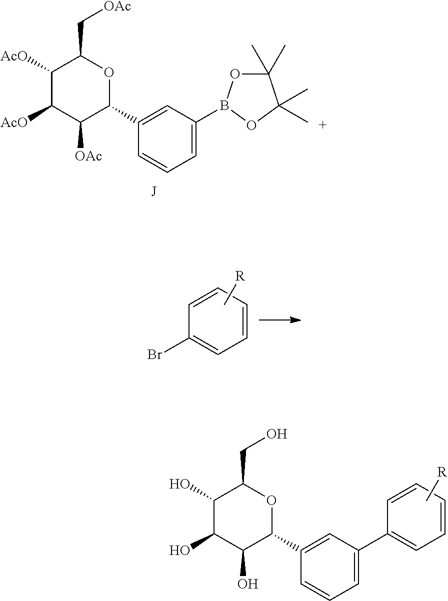

A microwave vial is charged with INTERMEDIATE J (1 eq.), the appropriate aryl bromide (1 eq.), Siliacat-DPP-Pd™ (0.26 mmol/g, 0.1 eq.), Cs₂CO₃ (2.2 eq.) and acetonitrile. The mixture is heated in the microwaved for 30 minutes at 100° C., filtered on Celite and concentrated to dryness. The residue is dissolved in MeOH, treated with 0.5 M NaOMe (0.5 eq.) and stirred overnight at room temperature. AcOH (0.5 eq.) is then added and the mixture is concentrated to dryness and purified by reverse phase preparative HPLC to give the desired product.

TABLE 5

| CMPD | | NMR | M + 1 |
|---|---|---|---|
| 174 | 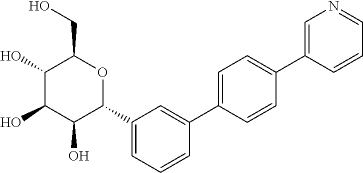 [SMILES: OC[C@H]1O[C@H](c2cccc(-c3ccc(-c4cccnc4)cc3)c2)[C@@H](O)[C@@H](O)[C@@H]1O] | ¹H NMR (400 MHz, DMSO-d6) δ 8.93 (d, J = 2.5 Hz, 1H), 8.56 (dd, J = 4.5, 1.7 Hz, 1H), 8.11 (dt, J = 7.8, 1.9 Hz, 1H), 7.79 (m, 5H), 7.60 (dt, J = 7.4, 1.7 Hz, 1H), 7.45 (m, 3H), 4.77 (m, 3H), 4.65 (s, 2H), 4.12 (m, 1H), 3.64 (m, 2H), 3.54 (t, J = 6.3 Hz, 1H), 3.45 (m, 2H). | 394.41 |
| 175 | 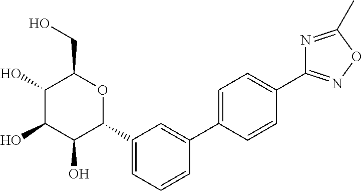 [SMILES: Cc1nc(-c2ccc(-c3cccc([C@H]4O[C@H](CO)[C@@H](O)[C@H](O)[C@@H]4O)c3)cc2)no1] | ¹H NMR (400 MHz, DMSO-d6) δ 8.05 (d, J = 8.4 Hz, 2H), 7.84 (d, J = 8.4 Hz, 2H), 7.77 (s, 1H), 7.62 (dt, J = 6.8, 2.1 Hz, 1H), 7.44 (m, 2H), 4.83 (d, J = 4.7 Hz, 1H), 4.78 (d, J = 5.3 Hz, 1H), 4.75 (d, J = 5.7 Hz, 1H), 4.64 (m, 2H), 4.08 (td, J = 5.9, 3.0 Hz, 1H), 3.65 (m, 2H), 3.54 (m, 1H), 3.46 (m, 2H), 2.65 (s, 3H). | 399.17 |
| 176 | 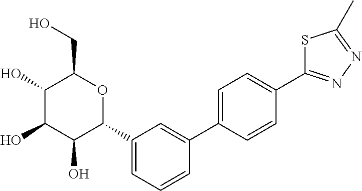 [SMILES: Cc1nnc(-c2ccc(-c3cccc([C@H]4O[C@H](CO)[C@@H](O)[C@H](O)[C@@H]4O)c3)cc2)s1] | ¹H NMR (400 MHz, Methanol-d4) δ 8.00 (d, J = 8.4 Hz, 2H), 7.86 (m, 1H), 7.81 (d, J = 8.4 Hz, 2H), 7.62 (dt, J = 6.5, 2.0 Hz, 1H), 7.49 (m, 2H), 5.03 (d, J = 4.0 Hz, 1H), 4.46 (dd, J = 4.1, 3.1 Hz, 1H), 3.85 (m, 2H), 3.74 (t, J = 7.6 Hz, 1H), 3.63 (dd, J = 7.8, 3.1 Hz, 1H), 3.55 (m, 1H), 2.81 (s, 3H). | 415.3 |
| 177 | 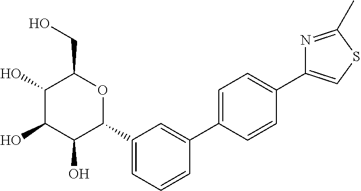 [SMILES: Cc1nc(-c2ccc(-c3cccc([C@H]4O[C@H](CO)[C@@H](O)[C@H](O)[C@@H]4O)c3)cc2)cs1] | ¹H NMR (400 MHz, Methanol-d4) δ 7.94 (d, J = 8.4 Hz, 2H), 7.82 (m, 1H), 7.71 (d, J = 8.4 Hz, 2H), 7.65 (s, 1H), 7.59 (td, J = 4.7, 1.8 Hz, 1H), 7.46 (dd, J = 4.9, 0.9 Hz, 2H), 5.03 (d, J = 3.8 Hz, 1H), 4.48 (t, J = 3.5 Hz, 1H), 3.85 (m, 2H), 3.74 (t, J = 7.8 Hz, 1H), 3.64 (dd, J = 7.9, 3.1 Hz, 1H), 3.54 (ddd, J = 7.7, 5.8, 4.0 Hz, 1H), 2.75 (s, 3H). | 414.3 |
| 178 | 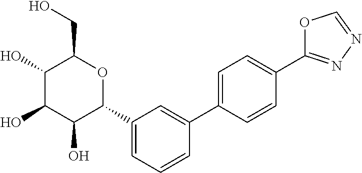 [SMILES: OC[C@H]1O[C@H](c2cccc(-c3ccc(-c4nnco4)cc3)c2)[C@@H](O)[C@@H](O)[C@@H]1O] | ¹H NMR (400 MHz, Methanol-d4) δ 8.95 (s, 1H), 8.07 (m, 2H), 7.80 (m, 3H), 7.56 (dt, J = 6.7, 2.1 Hz, 1H), 7.43 (m, 2H), 4.96 (d, J = 4.0 Hz, 1H), 4.39 (dd, J = 4.1, 3.0 Hz, 1H), 3.78 (m, 2H), 3.67 (t, J = 7.6 Hz, 1H), 3.56 (dd, J = 7.7, 3.1 Hz, 1H), 3.48 (td, J = 7.0, 3.5 Hz, 1H). | 385.34 |

### Preparation of Compound 179

### (2R,3S,4R,5S,6R)-2-(hydroxymethyl)-6-(3-(1-methyl-1H-benzo[d]imidazol-5-yl)phenyl)tetrahydro-2H-pyran-3,4,5-triol

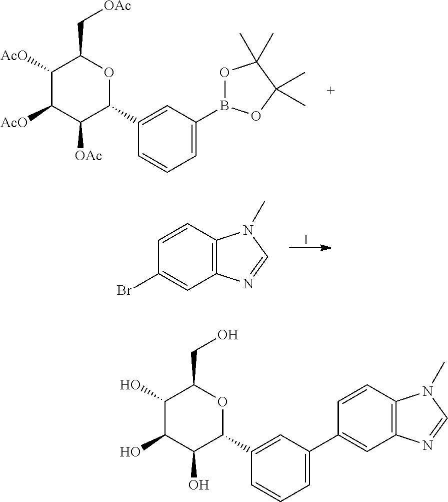

A microwave vial is charged with INTERMEDIATE J, 5-bromo-1-methyl-benzimidazole (12.1 mg, 0.0574 mmol), K₃PO₄ (37.5 mg, 0.177 mmol), PdCl₂(dppf)₂ (3.6 mg, 0.0044 mmol), DMF (1 mL) and heated in the microwave for 15 minutes at 120° C. The mixture is filtered on Millipore and concentrated to dryness. The residue is dissolved in MeOH (2 mL), treated with NaOMe (25% w/w, 10 μL 0.044 mmol) and stirred overnight at room temperature. The mixture is then filter on SCX-2 SPE column, the column is wash with 2M NH₃ in MeOH. The filtrate is concentrated to dryness and purified by reverse phase preparative HPLC to give the title compound (5.3 mg, 28%).

¹H NMR (400 MHz, Methanol-d4) δ 8.15 (s, 1H), 7.88 (m, 1H), 7.80 (m, 1H), 7.60 (m, 3H), 7.45 (m, 2H), 5.04 (d, J=3.7 Hz, 1H), 4.50 (t, J=3.5 Hz, 1H), 3.92 (s, 3H), 3.86 (m, 2H), 3.75 (t, J=7.9 Hz, 1H), 3.65 (dd, J=8.0, 3.1 Hz, 1H), 3.55 (m, 1H).

### Preparation of Compound 180

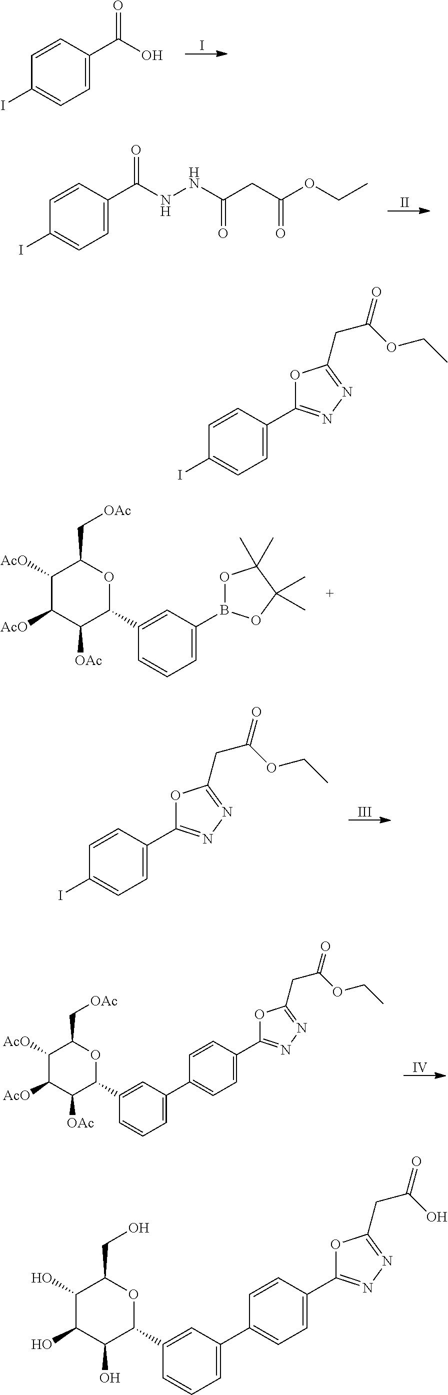

### Step I: ethyl 3-[2-(4-iodobenzoyl)hydrazino]-3-oxo-propanoate

To a solution of 4-iodobenzoic acid (2.021 g, 8.149 mmol), ethyl 3-hydrazino-3-oxo-propanoate (1.191 g, 8.149 mmol) in DMF (20 mL) are sequentially added HATU (3.408 g, 8.964 mmol), DIPEA (1.7 mL, 9.78 mmol) and the mixture is stirred overnight at room temperature. 80 mL of water is added to the mixture and the resulting solid is collected by filtration (2.617 g, 85%) to afford the title compound which is used as is in next step.

### Step II: ethyl 2-(5-(4-iodophenyl)-1,3,4-oxadiazol-2-yl)acetate

Ethyl 3-[2-(4-iodobenzoyl)hydrazino]-3-oxo-propanoate (2587 mg, 6.878 mmol) is suspended in POCl3 (25.9 mL) and the mixture is heated to reflux for 1 hour, cooled to room temperature, evaporated to dryness, the residue is diluted with 50 mL of DCM and poured on 100 g of crushed ice, the layers are separated and the aqueous phase is extracted with CH₂Cl₂ (4×50 mL). The combined organic layers are dried over Na₂SO₄, concentrated to dryness and purified by chromatography on silica gel (6 to 50% AcOEt) in hexanes to give the title compound (1.750 g, 71%).

### Step III: (2R,3R,4R,5R,6R)-2-(acetoxymethyl)-6-(4′-(5-(2-ethoxy-2-oxoethyl)-1,3,4-oxadiazol-2-yl)-[1,1′-biphenyl]-3-yl)tetrahydro-2H-pyran-3,4,5-triyltriacetate

A solution of ethyl 2-[5-(4-iodophenyl)-1,3,4-oxadiazol-2-yl]acetate (76.0 mg, 0.212 mmol), PdCl₂(dppf) (15.7 mg, 0.0193 mmol) and K₃PO₄ (81.85 mg, 0.3856 mmol) in DMF (2.1 mL) is heated to 100° C. overnight. The mixture is cooled to room temperature, water is added (4 mL) and the mixture is extracted with EtOAc (3×10 mL). The combined organic layers are washed with water (3×5 mL), brine (5 mL), dried on Na2SO4 and concentrated to dryness. The residue is purified by chromatography on silica gel (10 to 80% AcOEt in hexanes) to give the title compound (31.3 mg, 25%).

### Step IV: Compound 180

To a solution of (2R,3R,4R,5R,6R)-2-(acetoxymethyl)-6-(4′-(5-(2-ethoxy-2-oxoethyl)-1,3,4-oxadiazol-2-yl)-[1,1′-biphenyl]-3-yl)tetrahydro-2H-pyran-3,4,5-triyl triacetate (31 mg, 0.0485 mmol) in MeOH (1 mL) and water (0.5 mL) is added NaOH (2M, 24, 3 μL, 0.0485 mmol) and the mixture is stirred at room temperature for 3 days. The mixture is then filtered on an SCX-2 SPE cartridge, the filtrate is concentrated to dryness and purified by reverse phase preparative HPLC to give the title compound (3.6 mg, 17%).

### Preparation of Compound 181

### N-methyl-2-(5-(3′-((2R,3S,4R,5S,6R)-3,4,5-trihydroxy-6-(hydroxymethyl)tetrahydro-2H-pyran-2-yl)-[1,1′-biphenyl]-4-yl)-1,3,4-oxadiazol-2-yl)acetamide

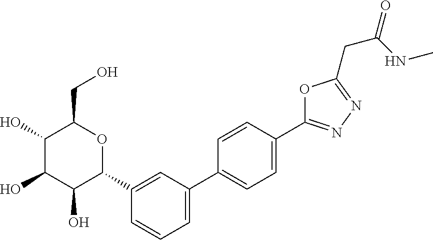 [SMILES: CNC(=O)Cc1nnc(-c2ccc(-c3cccc([C@H]4O[C@H](CO)[C@@H](O)[C@H](O)[C@@H]4O)c3)cc2)o1]

The title compound is prepared according to the procedure described for COMPOUND 180 using INTERMEDIATE J and 2-(5-(4-iodophenyl)-1,3,4-oxadiazol-2-yl)-N-methylacetamide prepared as described below.

¹H NMR (400 MHz, Methanol-d4) δ 8.53 (broad s, 1H), 8.10 (m, 2H), 7.85 (m, 3H), 7.62 (m, 1H), 7.49 (m, 2H), 5.03 (d, J=4.0 Hz, 1H), 4.46 (t, J=3.6 Hz, 1H), 4.01 (s, 2H), 3.85 (m, 2H), 3.75 (t, J=7.6 Hz, 1H), 3.58 (m, 8H), 3.43 (m, 2H), 3.35 (s, 3H). LC/MS: m/z=544.36 (M+H⁺)

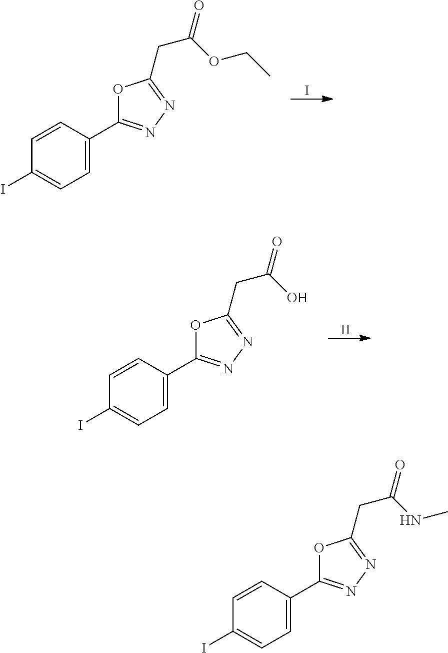

### Step I: 2-(5-(4-iodophenyl)-1,3,4-oxadiazol-2-yl)acetic acid

To a solution of ethyl 2-(5-(4-iodophenyl)-1,3,4-oxadiazol-2-yl)acetate (411 mg, 1.148 mmol) in dioxane (4.1 mL) is added NaOH (1M, 1.148 mmol) and the mixture is heated to 100° C. for 30 minutes. HCl (1M, 1.72 mL) is added, the mixture is concentrated to dryness, the residue is diluted with water (10 mL), extracted with EtOAc (3×15 mL), dried on Na₂SO₄ and concentrated to dryness to give the title compound (370 mg, 98%).

### Step II: 2-(5-(4-iodophenyl)-1,3,4-oxadiazol-2-yl)-N-methylacetamide

To a solution of 2-(5-(4-iodophenyl)-1,3,4-oxadiazol-2-yl)acetic acid 114.8 mg, 0.3478 mmol) in DMF (1.1 mL) is added sequentially methylamine (2M in THF, 191.3 μL, 0.3826 mmol), HATU (145.5 mg, 0.3826 mmol), DIPEA (72.7 μL, 0.4174 mmol) and the mixture is stirred overnight at room temperature. The mixture is diluted with water (5 mL), extracted with EtOAc (3×10 mL). The combined organic layers are washed with water (3×5 mL), brine (5 mL), dried on Na2SO4 and concentrated to dryness. The residue is purified by chromatography on silica gel (1 to 10% MeOH in CH₂Cl₂) to give the tilte compound (51 mg, 43%).

### Preparation of Compound 182

### N-(2-(2-methoxyethoxy)ethyl)-2-(5-(3′-((2R,3S,4R,5S,6R)-3,4,5-trihydroxy-6-(hydroxymethyl)tetrahydro-2H-pyran-2-yl)-[1,1′-biphenyl]-4-yl)-1,3,4-oxadiazol-2-yl)acetamide

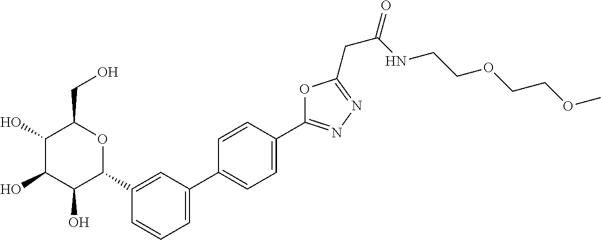 [SMILES: COCCOCCNC(=O)Cc1nnc(-c2ccc(-c3cccc([C@H]4O[C@H](CO)[C@@H](O)[C@H](O)[C@@H]4O)c3)cc2)o1]

The title compound is prepared according to the procedure described for COMPOUND 181 using INTERMEDIATE J and 2-(5-(4-iodophenyl)-1,3,4-oxadiazol-2-yl)-N-(2-(2-methoxyethoxy)ethyl)acetamide prepared as 2-(5-(4-iodophenyl)-1,3,4-oxadiazol-2-yl)-N-methylacetamide from COMPOUND 172 Step II.

### Preparation of Compound 183

### (2R,3S,4R,5S,6R)-2-(hydroxymethyl)-6-(3-(5-(5-methyl-1,3,4-oxadiazol-2-yl)pyridin-2-yl)phenyl)tetrahydro-2H-pyran-3,4,5-triol

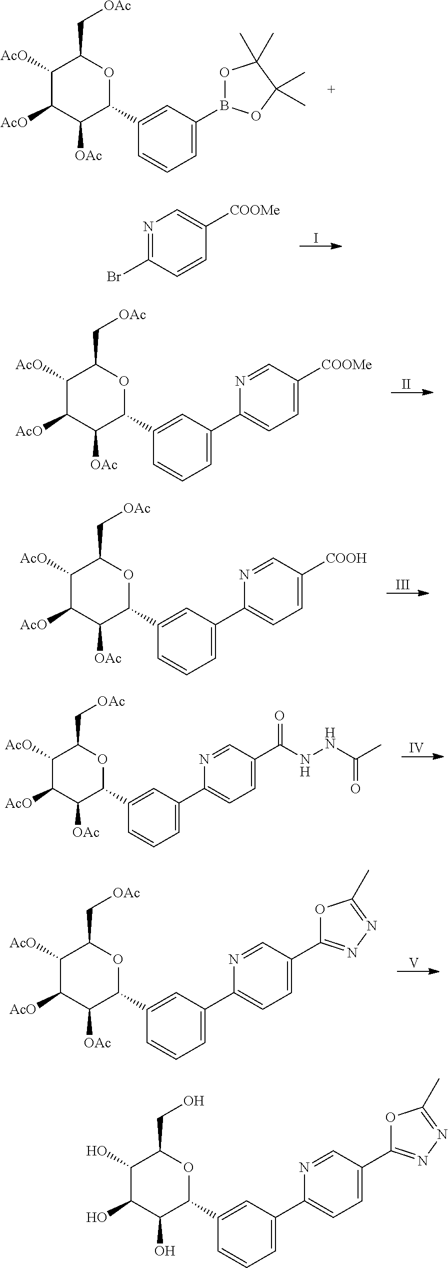

### Step I: (2R,3R,4R,5R,6R)-2-(acetoxymethyl)-6-(3-(5-(methoxycarbonyl)pyridin-2-yl)phenyl)tetrahydro-2H-pyran-3,4,5-triyltriacetate

A microwave vial is charged with INTERMEDIATE J (189 mg, 0.354 mmol), methyl 6-bromopyridine-3-carboxylate (76 mg, 0.354 mmol), Siliacat-DPP-Pd™ (0.26 mmol/g, 136 mg, 0.0354 mmol), Cs₂CO₃ (254 mg, 0.7781 mmol) and acetonitrile (3.8 mL). The mixture is heated in the microwaved for 30 minutes at 100° C., filtered on Celite and concentrated to dryness. The residue is purified by chromatography on silica gel (7 to 60% EtOAc in hexanes) to give the title compound (84 mg, 44%).

### Step II: 6-(3-((2R,3R,4R,5R,6R)-3,4,5-triacetoxy-6-(acetoxymethyl)tetrahydro-2H-pyran-2-yl)phenyl)nicotinic acid

To a solution of (2R,3R,4R,5R,6R)-2-(acetoxymethyl)-6-(3-(5-(methoxycarbonyl)pyridin-2-yl)phenyl)tetrahydro-2H-pyran-3,4,5-triyltriacetate (84 mg, 0.155 mmol) in MeOH (3 mL) is added NaOH (2M, 309.0 μL, 0.6180 mmol) and the mixture is stirred overnight at room temperature. HCl (4M) is then added until pH is between 1 and 2 and the mixture is evaporated to dryness. The residue is redissolved in pyridine (3 mL), DMAP (1.9 mg, 0.015 mmol), Ac₂O (87.5 μL, 0.927 mmol) are added, the mixture is stirred overnight at room temperature and concentrated to dryness. The residue is treated with HCl (1 M, 5 mL), extracted with CH₂Cl₂ (3×10 mL), dried on Na₂SO₄ and concentrated to dryness to give the title compound (75.5 mg, 92%).

Step III and IV are conducted as previously described in COMPOUND 171 Step I and II using acetohydrazide as reagent

This step is carried out using a procedure similar to what was previously described for the preparation of 2-(5-(4-iodophenyl)-1,3,4-oxadiazol-2-yl)-N-methylacetamide.

### Step V: Compound 183

The title compound is prepared from (2R,3R,4R,5R,6R)-2-(acetoxymethyl)-6-(3-(5-(5-methyl-1,3,4-oxadiazol-2-yl)pyridin-2-yl)phenyl)tetrahydro-2H-pyran-3,4,5-triyl triacetate according to the procedure described for COMPOUND 171 Step 1V.

¹H NMR (400 MHz, Methanol-d4) δ 9.22 (d, J=2.2 Hz, 1H), 8.43 (dd, J=8.4, 2.3 Hz, 1H), 8.23 (s, 1H), 8.10 (d, J=8.4 Hz, 1H), 8.03 (d, J=7.6 Hz, 1H), 7.61 (d, J=7.8 Hz, 1H), 7.53 (t, J=7.7 Hz, 1H), 5.04 (d, J=4.1 Hz, 1H), 4.47 (t, J=3.7 Hz, 1H), 3.86 (m, 2H), 3.76 (t, J=7.5 Hz, 1H), 3.65 (dd, J=7.7, 3.1 Hz, 1H), 3.55 (dt, J=7.0, 3.5 Hz, 1H), 2.64 (s, 3H).

### Preparation of Compound 184

### 1-(3′-((2R,3S,4R,5S,6R)-3,4,5-trihydroxy-6-(hydroxymethyl)tetrahydro-2H-pyran-2-yl)-[1,1′-biphenyl]-4-yl)ethanone

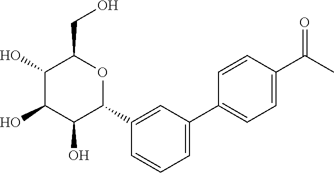 [SMILES: CC(=O)c1ccc(-c2cccc([C@H]3O[C@H](CO)[C@@H](O)[C@H](O)[C@@H]3O)c2)cc1]

The title compound is prepared according to the procedure described for COMPOUND 180 using INTERMEDIATE J and 1-(4-iodophenyl)ethanone.

¹H NMR (400 MHz, CD₃OD) δ 7.98 (d, J=8.3 Hz, 2H), 7.77 (d, J=1.3 Hz, 0H), 7.71 (d, J=8.4 Hz, 2H), 7.53 (m, 1H), 7.41 (m, 2H), 4.94 (d, J=4.0 Hz, 1H), 4.37 (dd, J=4.1, 3.0 Hz, 1H), 3.77 (m, 2H), 3.66 (t, J=7.6 Hz, 1H), 3.55 (dd, J=7.7, 3.1 Hz, 1H), 3.47 (dt, J=7.1, 3.5 Hz, 1H), 2.54 (s, 3H).

### Preparation of Compound 185

### 4-[3-[(2R,3S,4R,5S,6R)-3,4,5-trihydroxy-6-(hydroxymethyl)tetrahydropyran-2-yl]phenoxy]benzonitrile

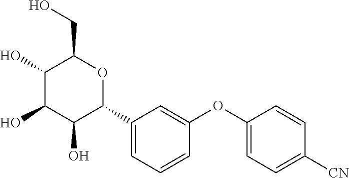 [SMILES: N#Cc1ccc(Oc2cccc([C@H]3O[C@H](CO)[C@@H](O)[C@H](O)[C@@H]3O)c2)cc1]

The title compound is prepared using similar procedure as described for COMPOUND 3 but using (4-cyanophenyl)boronic acid as the appropriate starting material. ¹H NMR (CD₃OD, 400 MHz): δ 7.76-7.58 (m, 2H), 7.44 (t, 1H), 7.35 (d, 1H), 7.24 (s, 1H), 7.14-6.91 (m, 3H), 4.93 (d, 1H), 4.34-4.25 (m, 1H), 3.90-3.65 (m, 3H), 3.59 (dd, 1H), 3.54-3.39 (m, 1H). LC-MS: m/z=358.3 (M+H⁺).

### Preparation of Compound 186

### 1-[4-[3-[(2R,3S,4R,5S,6R)-3,4,5-trihydroxy-6-(hydroxymethyl)tetrahydropyran-2-yl]phenoxy]phenyl]ethanone

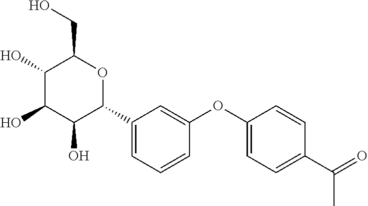 [SMILES: CC(=O)c1ccc(Oc2cccc([C@H]3O[C@H](CO)[C@@H](O)[C@H](O)[C@@H]3O)c2)cc1]

The title compound is prepared using similar procedure as described for COMPOUND 3 but using (4-acetylphenyl)boronic acid as the appropriate starting material. ¹H NMR (CD₃OD, 400 MHz): δ 8.04-7.90 (m, 2H), 7.43 (t, 1H), 7.33 (d, 1H), 7.23 (s, 1H), 7.09-6.92 (m, 3H), 4.92 (t, 1H), 4.40-4.24 (m, 1H), 3.94-3.65 (m, 3H), 3.59 (dd, 1H), 3.55-3.40 (m, 1H), 2.55 (s, 3H). LC-MS: m/z=375.4 (M+H⁺).

### Preparation of Compound 187

### 4-[2-methoxy-5-[(2R,3S,4R,5S,6R)-3,4,5-trihydroxy-6-(hydroxymethyl)tetrahydropyran-2-yl]phenoxy]-N-methyl-benzamide

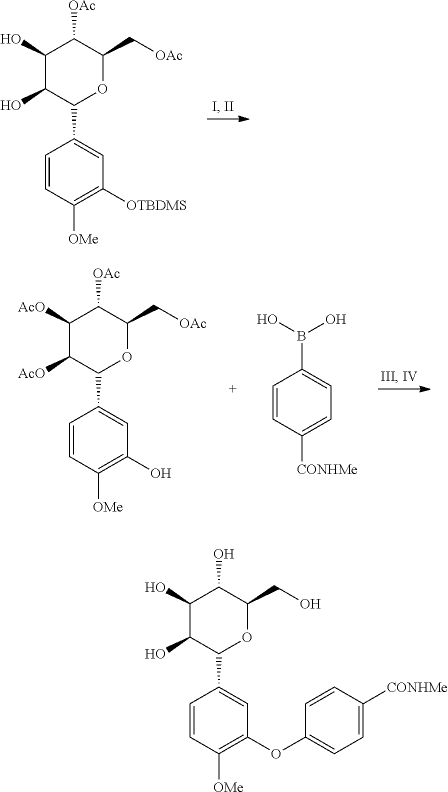

### Steps I, II: [(2R,3R,4R,5R,6R)-3,4,5-triacetoxy-6-(3-hydroxy-4-methoxy-phenyl)tetrahydropyran-2-yl]methyl acetate

To [(2R,3S,4R,5S,6R)-3-acetoxy-6-[3-[tert-butyl(dimethyl)silyl]oxy-4-methoxy-phenyl]-4,5-dihydroxy-tetrahydropyran-2-yl]methyl acetate (400 mg, 0.826 mmol) in Pyridine (8 ml) is added Ac₂O (194.7 μl, 2.065 mmol) followed by cat DMAP (10.08 mg, 0.0826 mmol). The reaction mixture is stirred at room temperature for 2 h, concentrated to dryness, diluted with CH₂Cl₂, washed with H₂O, brine. The organic phase was dried over Na₂SO₄, filtered and concentrated. To the previous residue in THF (8 ml) is added TBAF (1.65 ml of 1 M in THF, 1.65 mmol) followed by AcOH (47 μl, 0.826 mmol). The reaction mixture is stirred at room temperature overnight. The reaction mixture is concentrated, diluted with CH₂Cl₂, washed with H₂O, brine. The organic phase is dried over Na₂SO₄, filtered and concentrated.

LC-MS: m/z=455.4 (M+H⁺).

### Step III: [(2R,3R,4R,5R,6R)-3,4,5-triacetoxy-6-[4-methoxy-3-[4-(methylcarbamoyl)phenoxy]phenyl]tetrahydropyran-2-yl]methyl acetate

To a solution of [(2R,3R,4R,5R,6R)-3,4,5-triacetoxy-6-(3-hydroxy-4-methoxy-phenyl)tetrahydropyran-2-yl]methyl acetate (80 mg, 0.176 mmol) from COMPOUND 61 Step II, in CH₂Cl₂ (4.8 mL) is added [4-(methylcarbamoyl)phenyl]boronic acid (63 mg, 0.352 mmol), 4 A molecular sieves in powder (400 mg) followed by Cu(OAc)₂ (44.75 mg, 0.247 mmol). After stirring for 10 min, 2,6-lutidine (101.9 μl, 0.88 mmol) is added to the mixture. The reaction mixture is stirred at room temperature for 2 days, diluted with CH₂Cl₂, filtered on celite. After removal of the solvent under reduced pressure, the residue is separated on 10 g SNAP silica gel cartridge using a gradient of MeOH/CH₂Cl₂ 0-15% in 24 CV to afford the title compound (58 mg, 56%) as a colorless oil.

### Step IV: Compound 187

To a solution of the above-mentioned mixture (58 mg) in MeOH (1.6 ml) is added 2 drops of NaOMe (25% w/v in MeOH). After stirring at room temperature overnight the reaction mixture is neutralized with Amberlite IR120(H), the filtrate is concentrated and purified by reverse-phase prep-HPLC to afford the title compound (21.7 mg, 26.46%).

¹H NMR (CD₃OD, 400 MHz): δ 8.31 (s, 1H), 7.79-7.60 (m, 2H), 7.32 (dd, 1H), 7.22 (d, 1H), 7.13 (d, 1H), 6.97-6.75 (m, 2H), 4.89 (d, 1H), 4.31 (m, 1H), 3.86-3.66 (m, 6H), 3.61 (dd, 1H), 3.47 (td, 1H), 2.88 (d, 3H). LC-MS: m/z=420.3 (M+H⁺).

### Preparation of Compound 188

### 3-[2-methoxy-5-[(2R,3S,4R,5S,6R)-3,4,5-trihydroxy-6-(hydroxymethyl)tetrahydropyran-2-yl]phenoxy]-N-methyl-benzamide

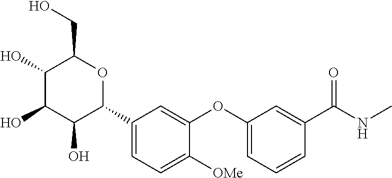 [SMILES: CNC(=O)c1cccc(Oc2cc([C@H]3O[C@H](CO)[C@@H](O)[C@H](O)[C@@H]3O)ccc2OC)c1]

The title compound is prepared as described for Compound 187 but using [3-(methylcarbamoyl)phenyl]boronic acid as the appropriate starting material.

¹H NMR (CD₃OD, 400 MHz): δ 7.48-7.37 (m, 1H), 7.38-7.22 (m, 3H), 7.23-7.14 (m, 1H), 7.12 (d, 1H), 6.99 (ddd, 1H), 4.91-4.87 (m, 1H), 4.38-4.26 (m, 1H), 3.87-3.67 (m, 6H), 3.65-3.56 (m, 1H), 3.46 (td, 1H), 2.86 (s, 3H). LC-MS: m/z=420.4 (M+H⁺).

### Preparation of Compound 189

### Methyl-4-[2-methoxy-5-[(2R,3S,4R,5S,6R)-3,4,5-trihydroxy-6-(hydroxymethyl)tetrahydropyran-2-yl]phenoxy]benzoate

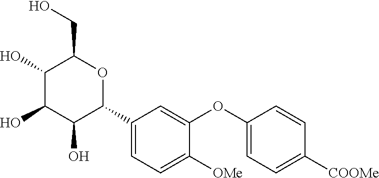 [SMILES: COC(=O)c1ccc(Oc2cc([C@H]3O[C@H](CO)[C@@H](O)[C@H](O)[C@@H]3O)ccc2OC)cc1]

The title compound is prepared as described for COMPOUND 187 but using (4-methoxycarbonylphenyl)boronic acid as the appropriate starting material.

¹H NMR (CD₃OD, 400 MHz): δ 8.02-7.80 (m, 2H), 7.34 (dd, 1H), 7.23 (d, 1H), 7.14 (d, 1H), 6.95-6.69 (m, 2H), 4.93 (m, 1H), 4.39-4.27 (m, 1H), 3.85 (s, 3H), 3.84-3.76 (m, 2H), 3.74 (s, 4H), 3.73-3.66 (m, 1H), 3.62 (dd, 1H), 3.48 (td, 1H). LC-MS: m/z=421.4 (M+H⁺).

### Preparation of Compound 190

### (2R,3S,4R,5S,6R)-2-(2-fluoro-3-hydroxy-phenyl)-6-(hydroxymethyl)tetrahydropyran-3,4,5-triol

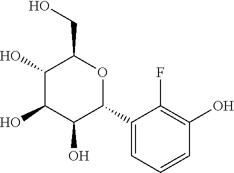 [SMILES: OC[C@H]1O[C@H](c2cccc(O)c2F)[C@@H](O)[C@@H](O)[C@@H]1O]

The title compound is prepared as described for COMPOUND 67 but using (2-fluoro-3-hydroxyphenyl)boronic acid as the appropriate starting material.

¹H NMR (CD₃OD, 400 MHz): δ 7.01-6.83 (m, 2H), 6.76 (td, 1H), 5.05 (d, 1H), 4.16 (dd, 1H), 3.86 (dd, 1H), 3.79-3.65 (m, 3H), 3.65-3.49 (m, 1H).

LC-MS: m/z=297.2 (M+Na⁺)

### Preparation of Compound 191

### (2R,3S,4R,5S,6R)-2-(hydroxymethyl)-6-[5-hydroxy-2-(trifluoromethoxy)phenyl]tetrahydropyran-3,4,5-triol

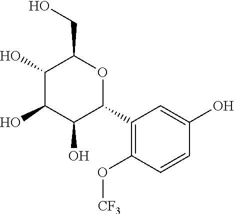 [SMILES: OC[C@H]1O[C@H](c2cc(O)ccc2OC(F)(F)F)[C@@H](O)[C@@H](O)[C@@H]1O]

The title compound is prepared as described for COMPOUND 67 but using (5-hydroxy-2-(trifluoromethoxy)phenyl)boronic acid as the appropriate starting material.

¹H NMR (CD₃OD, 400 MHz): δ 7.17-6.97 (m, 2H), 6.73 (dd, 1H), 5.09 (d, 1H), 4.11 (dd, 1H), 4.00-3.80 (m, 4H), 3.73 (dt, 1H). LC-MS: m/z=342.3 (M+H⁺)

### Preparation of Compound 192

### (2R,3S,4R,5S,6R)-2-(2-fluoro-5-hydroxy-phenyl)-6-(hydroxymethyl)tetrahydropyran-3,4,5-triol

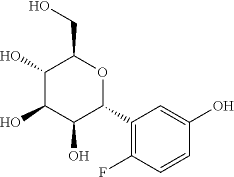 [SMILES: OC[C@H]1O[C@H](c2cc(O)ccc2F)[C@@H](O)[C@@H](O)[C@@H]1O]

The title compound is prepared as described for COMPOUND 67 but using (3-hydroxy-6-fluorophenyl)boronic acid as the appropriate starting material.

¹H NMR (CD₃OD, 400 MHz): δ 6.89 (dd, 1H), 6.79 (dd, 1H), 6.66-6.50 (m, 1H), 4.98 (d, 1H), 4.15 (dd, 1H), 3.86 (dd, 1H), 3.78-3.49 (m, 4H). LC-MS: m/z=275.2 (M+H⁺)

### Preparation of Compound 193

### (2R,3S,4R,5S,6R)-2-(4-fluoro-3-hydroxy-phenyl)-6-(hydroxymethyl)tetrahydropyran-3,4,5-triol

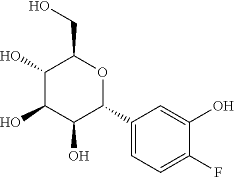 [SMILES: OC[C@H]1O[C@H](c2ccc(F)c(O)c2)[C@@H](O)[C@@H](O)[C@@H]1O]

The title compound is prepared as described for COMPOUND 67 but using (3-hydroxy-4-fluorophenyl)boronic acid as the appropriate starting material.

¹H NMR (CD₃OD, 400 MHz): δ 7.12-6.95 (m, 2H), 6.95-6.75 (m, 1H), 4.85 (d, 1H), 4.33 (t, 1H), 3.87-3.77 (m, 2H), 3.71 (t, 1H), 3.57 (dd, 1H), 3.53-3.33 (m, 1H). LC-MS: m/z=275.2 (M+H⁺)

### Preparation of Compound 194

### (2R,3S,4R,5S,6R)-2-(hydroxymethyl)-6-(3-hydroxy-5-methyl-phenyl)tetrahydropyran-3,4,5-triol

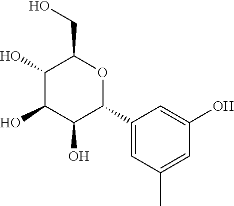 [SMILES: Cc1cc(O)cc([C@H]2O[C@H](CO)[C@@H](O)[C@H](O)[C@@H]2O)c1]

The title compound is prepared as described for COMPOUND 67 but using (3-hydroxy-5-methylphenyl)boronic acid as the appropriate starting material.

¹H NMR (CD₃OD, 400 MHz): δ 6.74 (s, 1H), 6.69 (s, 1H), 6.51 (s, 1H), 4.86-4.86 (m, 1H), 4.39 (t, 1H), 3.86-3.61 (m, 3H), 3.55 (dd, 1H), 3.45 (ddd, 1H), 2.25 (s, 3H). LC-MS: m/z=271.2 (M+H⁺)

### Preparation of Compound 195

### N-methyl-4-[4-(trifluoromethoxy)-3-[(2R,3S,4R,5S,6R)-3,4,5-trihydroxy-6-(hydroxymethyl)tetrahydropyran-2-yl]phenoxy]benzamide

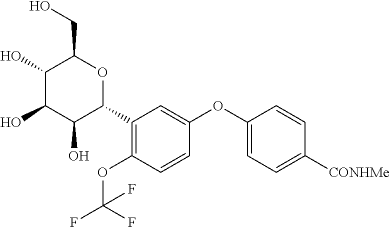

The title compound is prepared using similar procedure as described for COMPOUND 3 but using the intermediate [(2R,3S,4R,5S,6R)-3-acetoxy-4,5-dihydroxy-6-[5-[4-(methylcarbamoyl)phenoxy]-2-(trifluoromethoxy)phenyl]tetrahydropyran-2-yl]methyl acetate from COMPOUND 182 as the appropriate starting material.

¹H NMR (CD₃OD, 400 MHz): δ 7.92-7.70 (m, 2H), 7.43 (dd, 1H), 7.32 (d, 1H), 7.13-6.93 (m, 3H), 5.15 (dd, 1H), 4.13-3.57 (m, 6H), 2.89 (s, 3H). LC-MS: m/z=474.4 (M+H⁺).

### Preparation of Compound 196

### 4-[2-fluoro-5-[(2R,3S,4R,5S,6R)-3,4,5-trihydroxy-6-(hydroxymethyl)tetrahydropyran-2-yl]phenoxy]-N-methyl-benzamide

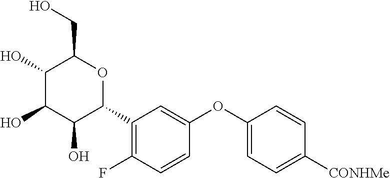

The title compound is prepared using similar procedure as described for COMPOUND 3 but using the intermediate [(2R,3S,4R,5S,6R)-3-acetoxy-6-(2-fluoro-5-hydroxy-phenyl)-4,5-dihydroxy-tetrahydropyran-2-yl]methyl acetate from COMPOUND 183 as the appropriate starting material.

¹H NMR (CD₃OD, 400 MHz): δ 7.87-7.71 (m, 2H), 7.35 (t, 2H), 7.32-7.14 (m, 1H), 6.97 (d, 2H), 4.86-4.85 (m, 1H), 4.21 (dd, 1H), 3.87 (dd, 1H), 3.81-3.66 (m, 2H), 3.63 (dd, 1H), 3.57-3.38 (m, 1H), 2.88 (s, 3H). LC-MS: m/z=408.3 (M+H⁺).

### Preparation of Compound 197

### 4-[2-fluoro-5-[(2R,3S,4R,5S,6R)-3,4,5-trihydroxy-6-(hydroxymethyl)tetrahydropyran-2-yl]phenoxy]-N-methyl-benzamide

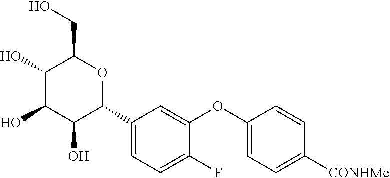

The title compound is prepared using similar procedure as described for COMPOUND 3 but using the intermediate [(2R,3S,4R,5S,6R)-3-acetoxy-6-[2-fluoro-3-[4-(methylcarbamoyl)phenoxy]phenyl]-4,5-dihydroxy-tetrahydropyran-2-yl]methyl acetate from COMPOUND 184 as the appropriate starting material.

¹H NMR (CD₃OD, 400 MHz): δ 8.01-7.52 (m, 2H), 7.35 (dd, 1H), 7.25-6.44 (m, 4H), 5.12 (d, 1H), 4.05 (ddd, 2H), 3.89-3.76 (m, 2H), 3.72 (dd, 2H), 2.88 (s, 3H). LC-MS: m/z=408.3 (M+H⁺).

### Preparation of Compound 198

### 4-[2-fluoro-3-[(2R,3S,4R,5S,6R)-3,4,5-trihydroxy-6-(hydroxymethyl)tetrahydropyran-2-yl]phenoxy]-N-methyl-benzamide

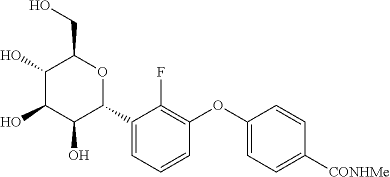

The title compound is prepared using similar procedure as described for COMPOUND 3 but using the intermediate [(2R,3S,4R,5S,6R)-3-acetoxy-6-(2-fluoro-3-hydroxy-phenyl)-4,5-dihydroxy-tetrahydropyran-2-yl]methyl acetate from COMPOUND 181 as the appropriate starting material.

¹H NMR (CD₃OD, 400 MHz): δ 7.97-7.62 (m, 2H), 7.51 (t, 1H), 7.17 (dt, 2H), 6.97 (dd, 2H), 5.16 (d, 1H), 4.30-3.93 (m, 2H), 3.90-3.59 (m, 4H), 2.89 (d, 3H).

LC-MS: m/z=408.4 (M+H⁺)

### Preparation of Compound 199

### N-methyl-4-[3-methyl-5-[(2R,3S,4R,5S,6R)-3,4,5-trihydroxy-6-(hydroxymethyl)tetrahydropyran-2-yl]phenoxy]benzamide

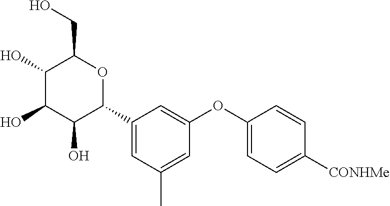

The title compound is prepared using similar procedure as described for COMPOUND 3 but using the intermediate [(2R,3S,4R,5S,6R)-3-acetoxy-4,5-dihydroxy-6-(3-hydroxy-5-methyl-phenyl)tetrahydropyran-2-yl]methyl acetate from COMPOUND 185 as the appropriate starting material.

¹H NMR (CD₃OD, 400 MHz): δ 7.73-7.61 (m, 2H), 7.05 (s, 1H), 6.99-6.83 (m, 3H), 6.72 (s, 1H), 5.39 (s, 1H), 4.29-4.14 (m, 1H), 3.82-3.56 (m, 3H), 3.50 (dd, 1H), 3.39 (td, 1H), 2.81 (s, 3H), 2.26 (s, 3H). LC-MS: m/z=404.3 (M+H⁺).

### Preparation of Compound 200

### 3-[2-fluoro-3-[(2R,3S,4R,5S,6R)-3,4,5-trihydroxy-6-(hydroxymethyl)tetrahydropyran-2-yl]phenoxy]-N-methyl-benzamide

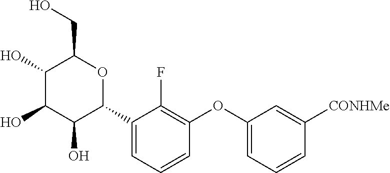

The title compound is prepared using similar procedure as described for COMPOUND 3 but using the intermediate [(2R,3S,4R,5S,6R)-3-acetoxy-6-(2-fluoro-3-hydroxy-phenyl)-4,5-dihydroxy-tetrahydropyran-2-yl]methyl acetate from COMPOUND 181 and [3-(methylcarbamoyl)phenyl]boronic acid as the appropriate starting materials.

¹H NMR (CD₃OD, 400 MHz): δ 7.49 (dt, 2H), 7.43-7.28 (m, 2H), 7.20 (t, 1H), 7.09 (dd, 2H), 5.16 (d, 1H), 4.14 (dd, 1H), 4.04 (dd, 1H), 3.92-3.60 (m, 4H), 2.86 (s, 3H). LC-MS: m/z=408.3 (M+H⁺).

### Preparation of Compound 201

### N-methyl-3-[4-(trifluoromethoxy)-3-[(2R,3S,4R,5S,6R)-3,4,5-trihydroxy-6-(hydroxymethyl)tetrahydropyran-2-yl]phenoxy]benzamide

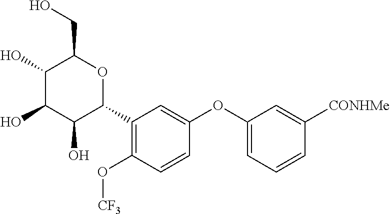

The title compound is prepared using similar procedure as described for COMPOUND 3 but using the intermediate [(2R,3S,4R,5S,6R)-3-acetoxy-4,5-dihydroxy-6-[5-hydroxy-2-(trifluoromethoxy)phenyl]tetrahydropyran-2-yl]methyl acetate from COMPOUND 182 and [3-(methylcarbamoyl)phenyl]boronic acid as the appropriate starting materials.

¹H NMR (CD₃OD, 400 MHz): δ 7.59-7.40 (m, 1H), 7.42-7.27 (m, 3H), 7.22 (dd, 1H), 7.14-7.03 (m, 1H), 6.94 (dd, 1H), 5.07 (d, 1H), 3.98-3.84 (m, 2H), 3.84-3.59 (m, 4H), 2.81 (d, 3H). LC-MS: m/z=474.3 (M+H⁺).

### Preparation of Compound 202

### 3-[4-fluoro-3-[(2R,3S,4R,5S,6R)-3,4,5-trihydroxy-6-(hydroxymethyl)tetrahydropyran-2-yl]phenoxy]-N-methyl-benzamide

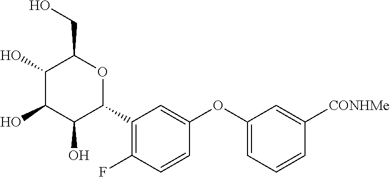

The title compound is prepared using similar procedure as described for COMPOUND 3 but using the intermediate [(2R,3S,4R,5S,6R)-3-acetoxy-6-(2-fluoro-5-hydroxy-phenyl)-4,5-dihydroxy-tetrahydropyran-2-yl]methyl acetate from COMPOUND 183 and [3-(methylcarbamoyl)phenyl]boronic acid as the appropriate starting materials.

¹H NMR (CD₃OD, 400 MHz): δ 7.46-7.38 (m, 1H), 7.38-7.27 (m, 2H), 7.23 (dd, 1H), 7.09-6.97 (m, 2H), 6.95-6.82 (m, 1H), 5.04 (d, 1H), 4.03 (dd, 1H), 3.93 (dd, 1H), 3.78-3.68 (m, 2H), 3.68-3.55 (m, 2H), 2.79 (s, 3H). LC-MS: m/z=408.3 (M+H⁺).

### Preparation of Compound 203

### 3-[2-fluoro-5-[(2R,3S,4R,5S,6R)-3,4,5-trihydroxy-6-(hydroxymethyl)tetrahydropyran-2-yl]phenoxy]-N-methyl-benzamide

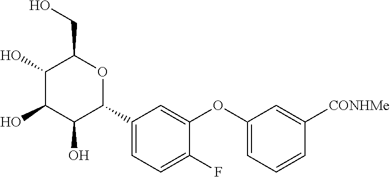

The title compound is prepared using similar procedure as described for COMPOUND 3 but using the intermediate [(2R,3S,4R,5S,6R)-3-acetoxy-6-(4-fluoro-3-hydroxy-phenyl)-4,5-dihydroxy-tetrahydropyran-2-yl]methyl acetate from COMPOUND 184 and [3-(methylcarbamoyl)phenyl]boronic acid as the appropriate starting materials.

¹H NMR (CD₃OD, 400 MHz): δ 7.61-7.47 (m, 1H), 7.47-7.35 (m, 2H), 7.36-7.18 (m, 3H), 7.11 (dd, 1H), 4.86-4.85 (m, 1H), 4.22 (dd, 1H), 3.85 (dd, 1H), 3.78-3.69 (m, 2H), 3.62 (dd, 1H), 3.57-3.41 (m, 1H), 2.87 (s, 3H). LC-MS: m/z=408.4 (M+H⁺).

### Preparation of Compound 204

### N-methyl-3-[3-methyl-5-[(2R,3S,4R,5S,6R)-3,4,5-trihydroxy-6-(hydroxymethyl)tetrahydropyran-2-yl]phenoxy]benzamide

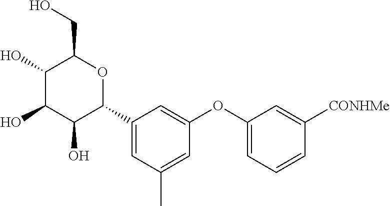

The title compound is prepared using similar procedure as described for COMPOUND 3 but using the intermediate [(2R,3S,4R,5S,6R)-3-acetoxy-4,5-dihydroxy-6-(3-hydroxy-5-methyl-phenyl)tetrahydropyran-2-yl]methyl acetate from COMPOUND 185 and [3-(methylcarbamoyl)phenyl]boronic acid as the appropriate starting materials.

¹H NMR (CD₃OD, 400 MHz): δ 7.56-7.41 (m, 1H), 7.36 (dd, 2H), 7.14-6.99 (m, 2H), 6.90 (s, 1H), 6.71 (s, 1H), 5.43 (s, 1H), 4.27 (t, 1H), 3.87-3.60 (m, 3H), 3.53 (dd, 1H), 3.41 (td, 1H), 2.82 (s, 3H), 2.28 (s, 3H). LC-MS: m/z=404.4 (M+H⁺).

### Preparation of Compound 205

### (2R,3S,4R,5S,6R)-2-(hydroxymethyl)-6-(4-phenoxyphenyl)tetrahydropyran-3,4,5-triol

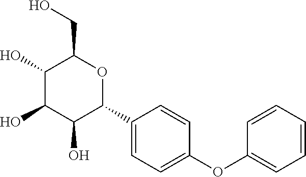 [SMILES: OC[C@H]1O[C@H](c2ccc(Oc3ccccc3)cc2)[C@@H](O)[C@@H](O)[C@@H]1O]

The title compound is prepared using similar procedure as described for COMPOUND 3 but using [(2R,3S,4R,5S,6R)-3-acetoxy-4,5-dihydroxy-6-(4-hydroxyphenyl)tetrahydropyran-2-yl]methyl acetate from INTERMEDIATE L Step II and phenylboronic acid as the appropriate starting materials.

¹H NMR (CD₃OD, 400 MHz): δ 7.37 (d, 2H), 7.31-7.13 (m, 2H), 7.12-6.95 (m, 1H), 6.95-6.78 (m, 4H), 4.84 (t, 1H), 4.30 (t, 1H), 3.78-3.68 (m, 2H), 3.65 (t, 1H), 3.52 (dd, 1H), 3.39 (ddd, 1H). LC-MS: m/z=355.3 (M+Na⁺).

### Preparation of Compound 206

### (2R,3S,4R,5S,6R)-2-[3-(2-fluoroethoxy)phenyl]-6-(hydroxymethyl)tetrahydropyran-3,4,5-triol

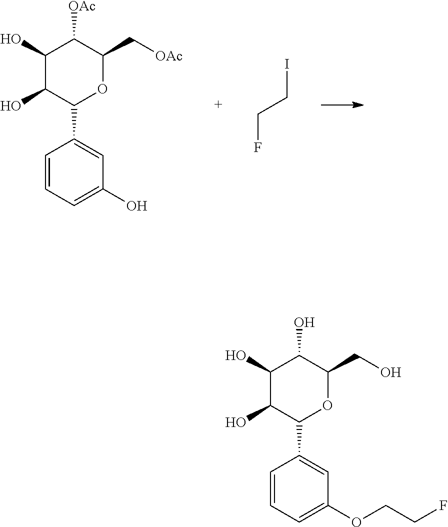

To a mixture of INTERMEDIATE A (30 mg, 0.088 mmol) and 1-fluoro-2-iodo-ethane (45.99 mg, 0.265 mmol) in DMF (600 μl) is added Cs₂CO₃ (143.6 mg, 0.44 mmol). The reaction mixture is heated at 70° C. for 20 h, concentrated to dryness. The reaction mixture is diluted with MeOH, treated with 2 drops catalytic NaOMe (25% w/v in MeOH). After 20 h at room temperature, the reaction mixture is neutralized with Amberlite IR120(H). After filtration, washing with CH₂Cl₂/MeOH 9/1, the solvent is removed under reduced pressure and the residue purified by reverse-phase prep-HPLC to give the title compound as a white powder (5.2 mg, 17%).

¹H NMR (400 MHz, CD₃OD) δ 7.19 (t, 1H), 7.02 (s, 1H), 6.95 (d, 1H), 6.78 (dd, 1H), 4.85 (d, 1H), 4.71-4.64 (m, 1H), 4.55 (dd, 1H), 4.33 (t, 1H), 4.21-4.12 (m, 1H), 4.12-4.03 (m, 1H), 3.78-3.67 (m, 2H), 3.62 (t, 1H), 3.47 (dd, 1H), 3.43-3.33 (m, 1H). LC-MS: m/z=325.3 (M+Na⁺).

### Preparation of Compound 207 to 226 in Table 6

COMPOUNDs 198 to 217 are prepared from INTERMEDIATE A according to the procedure described for COMPOUND 197 but using the appropriate commercially available bromo or iodo alkylating reagent. In some cases DMF is replaced by NMP as solvent.

TABLE 6

| | | LC-MS: |
|---|---|---|
| CMPD | Structure and Name | m/z (M + H⁺) |
| 207 | 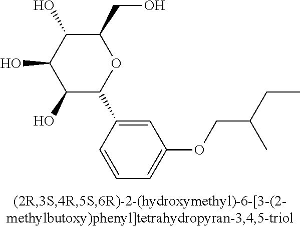 | 349.3 (M + Na⁺) |
| 208 | 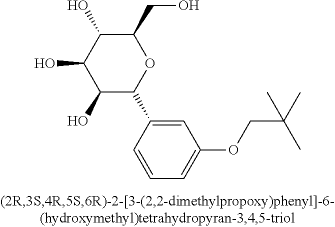 | 349.3 (M + Na⁺) |
| 209 | 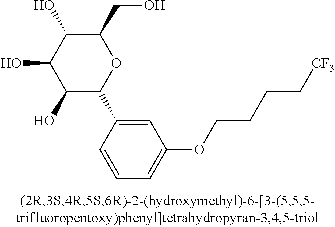 | 381.3 |
| 210 | 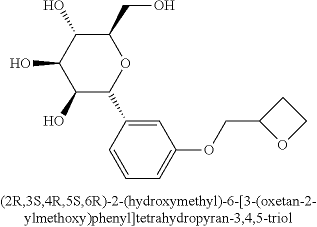 | 349.3 (M + Na⁺) |
| 211 | 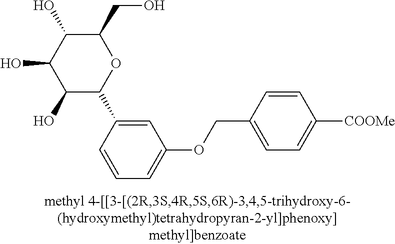 | 405.3 |
| 212 | 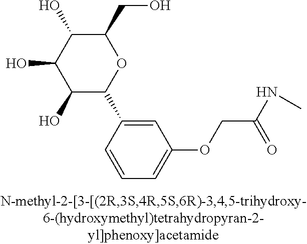 | 428.3 |
| 213 | 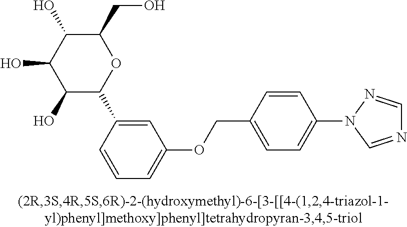 | 414.4 |
| 214 | 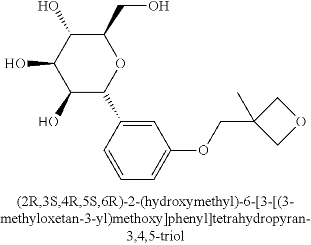 | 341.4 |
| 215 | 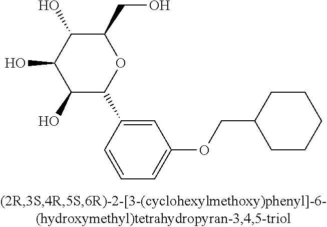 | 353.4 |
| 216 | 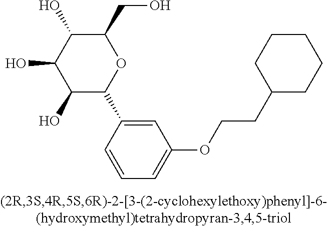 | 367.4 |
| 217 | 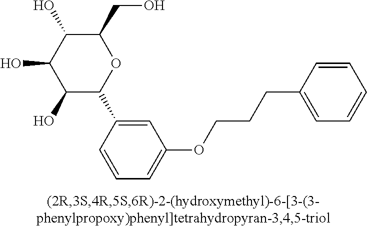 | 375.4 |
| 218 | 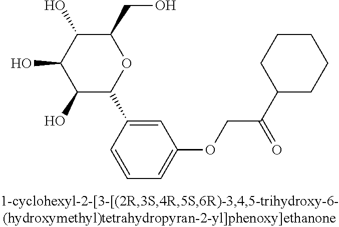 | 381.4 |
| 219 | 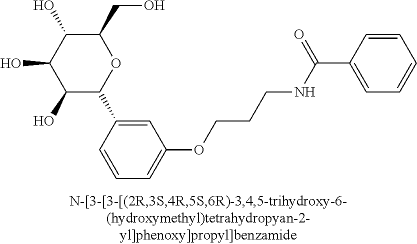 | 418.4 |
| 220 | 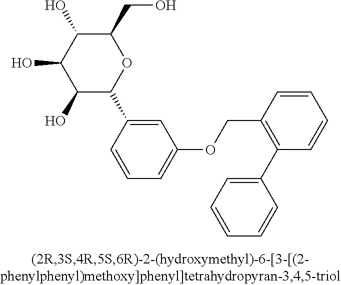 | 423.4 |
| 221 | 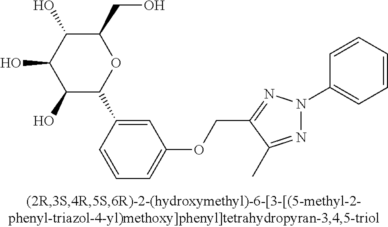 | 428.4 |
| 222 | 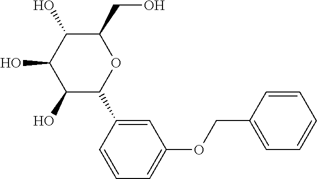 | 347.3 |
| 223 | 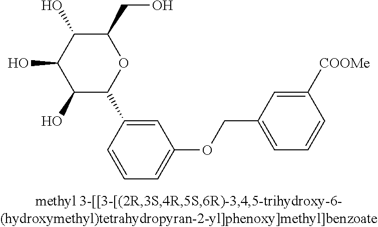 | 427.4 (M + Na⁺) |
| 224 | 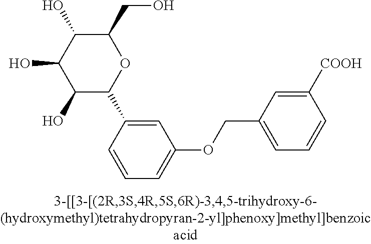 | 391.4 |
| 225 | 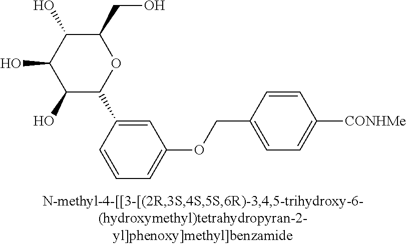 | 404.4 |
| 226 | 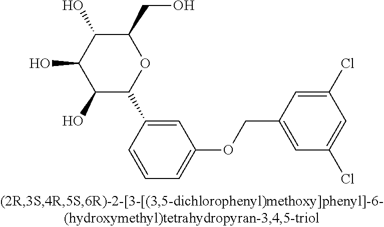 | 415.3 |

### Preparation of Compound 227

### N-methyl-3-[[3-[(2R,3S,4R,5S,6R)-3,4,5-trihydroxy-6-(hydroxymethyl)tetrahydropyran-2-yl]phenoxy]methyl]benzamide

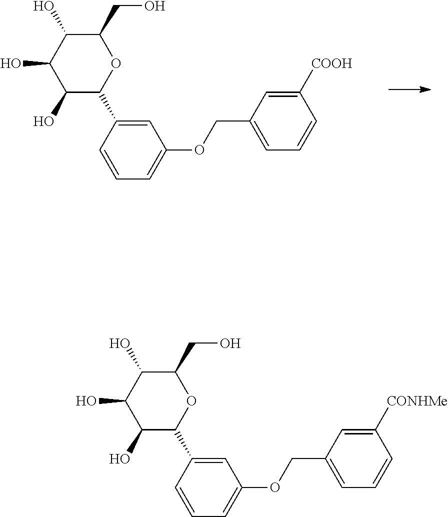

To COMPOUND 224 (11.2 mg, 0.0281 mmol) in NMP (220 μl) is added a THF solution of methylamine (17 μl of 2 M, 0.0338 mmol), 2,6-lutidine (6.5 μl, 0.0563 mmol) followed by HATU (13.90 mg, 0.0366 mmol). The reaction mixture is stirred at RT overnight and directly purified by reverse-phase prep-HPLC to afford N-methyl-3-[[3-[(2R,3S,4R,5S,6R)-3,4,5-trihydroxy-6-(hydroxymethyl)tetrahydropyran-2-yl]phenoxy]methyl]benzamide (8.6 mg, 69%).

¹H NMR (400 MHz, CD₃OD) δ 7.89 (s, 1H), 7.74 (d, 1H), 7.60 (d, 1H), 7.46 (t, 1H), 7.27 (t, 1H), 7.17 (s, 1H), 7.03 (d, 1H), 6.92 (dd, 1H), 5.14 (s, 2H), 4.93 (d, 1H), 4.41 (t, 1H), 3.89-3.74 (m, 2H), 3.75-3.58 (m, 1H), 3.54 (dd, 1H), 3.44 (dt, 1H), 2.90 (s, 3H). LC-MS: m/z=404.4 (M+H⁺).

### Preparation of Compound 228 to 251 in Table 7

COMPOUNDs 219 to 242 are prepared, according to the procedure described for COMPOUND 197 but using the reagent described in INTERMEDIATE L Step II and the appropriate commercially available bromo or iodo alkylating reagent.

### Preparation of Compound 219

TABLE 7

| | | LC-MS: |
|---|---|---|
| COMPOUND | Structure and Name | m/z (M + H⁺) |
| 228 | 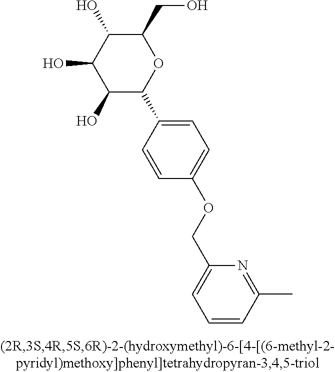 | 362.3 |
| 229 | 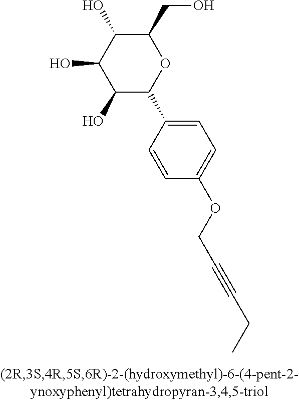 | 345.3 (M + Na⁺) |
| 230 | 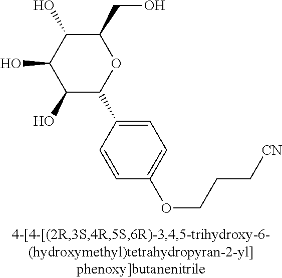 | 324.3 |
| 231 | 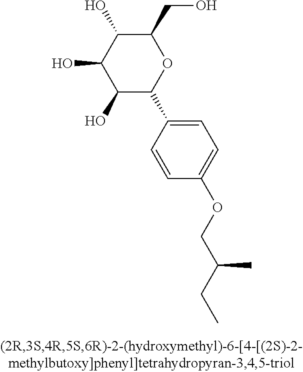 | 349.3 (M + Na⁺) |
| 232 | 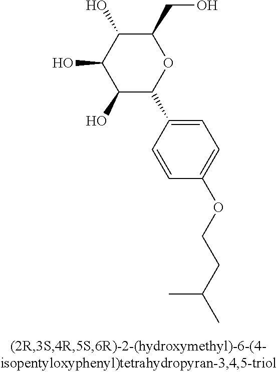 | 349.3 (M + Na⁺) |
| 233 | 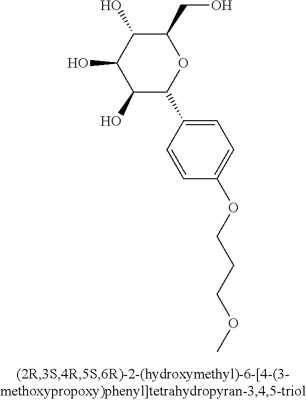 | 351.3 (M + Na⁺) |
| 234 | 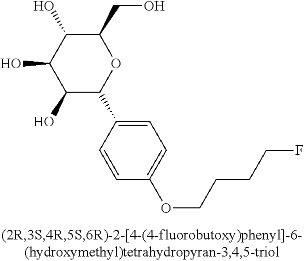 | 353.3 (M + Na⁺) |
| 235 | 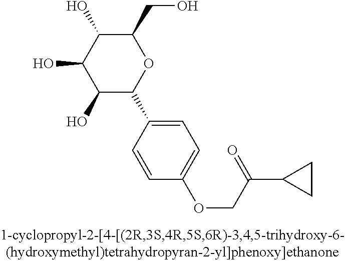 | 361.3 (M + Na⁺) |
| 236 | 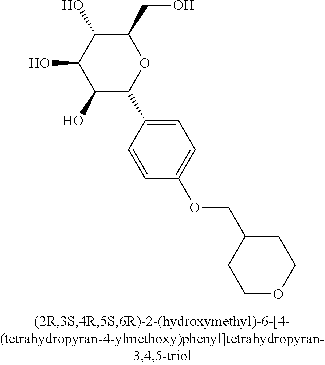 | 377.3 (M + Na⁺) |
| 237 | 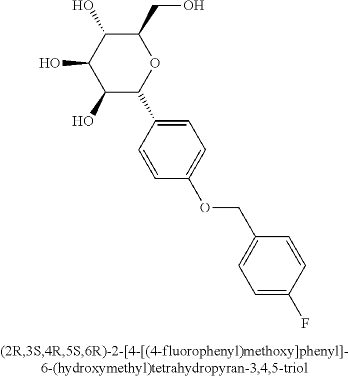 | 387.4 (M + Na⁺) |
| 238 | 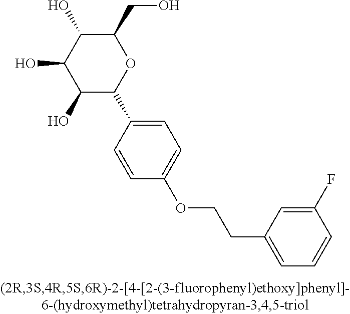 | 401.3 (M + Na⁺) |
| 239 | 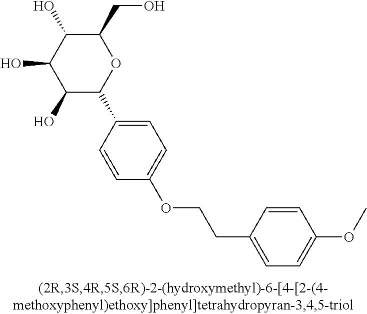 | 413.4 (M + Na⁺) |
| 240 | 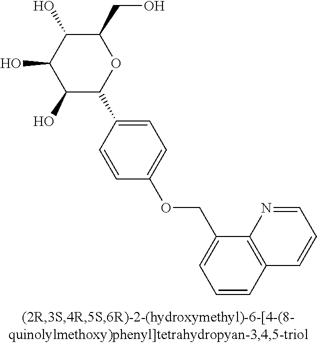 | 398.4 |
| 241 | 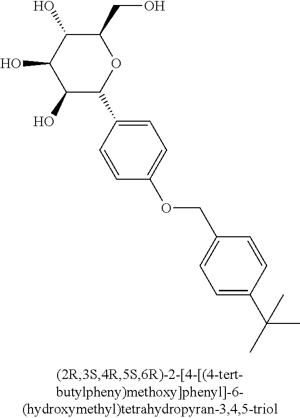 | 425.4 (M + Na⁺) |
| 242 | 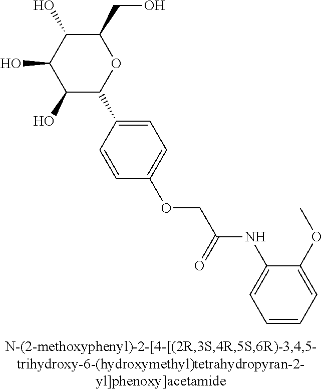 | 420.3 |
| 243 | 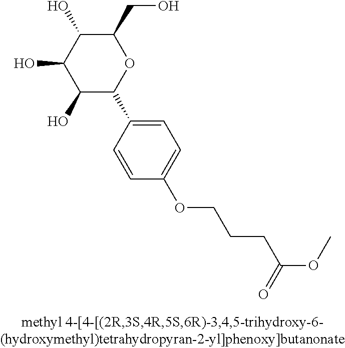 | 379.3 (M + Na⁺) |
| 244 | 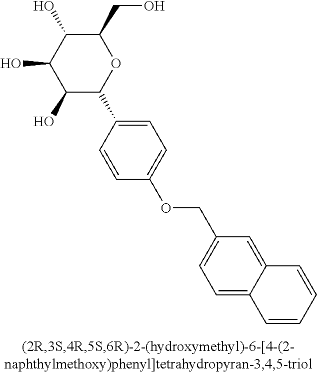 | 419.4 (M + Na⁺) |
| 245 | 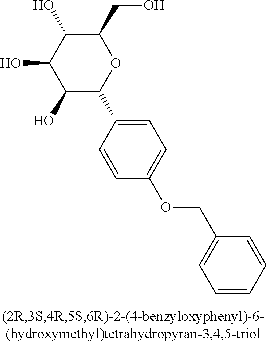 | 369.4 (M + Na⁺) |
| 246 | 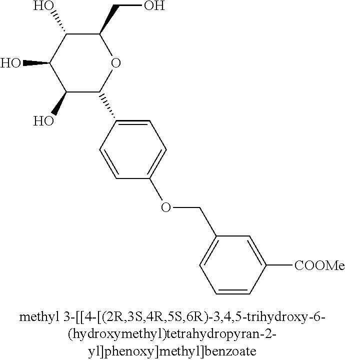 | 427.5 (M + Na⁺) |
| 247 | 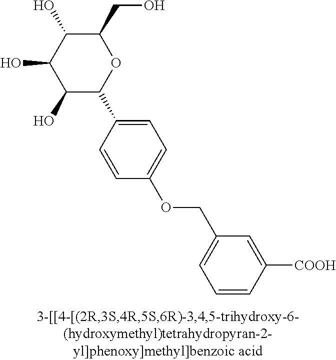 | 413.4 (M + Na⁺) |
| 248 | 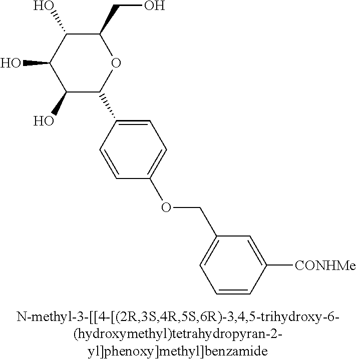 | 404.4 |
| 249 | 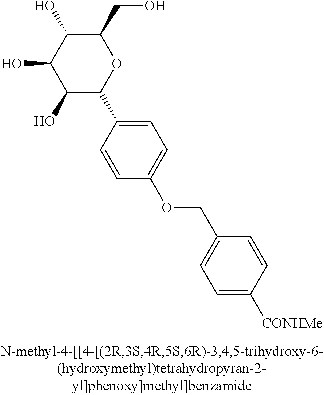 | 404.4 |
| 250 | 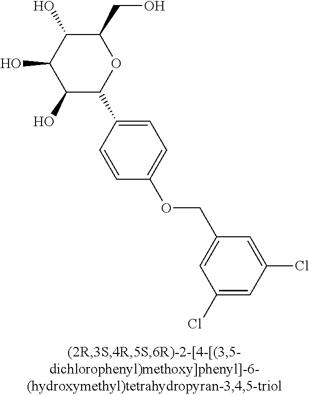 | 415.2 |
| 251 | 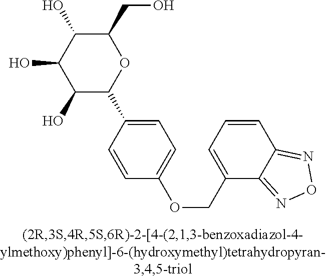 | 411.5 (M + Na⁺) |

### Preparation of Compound 252

### (2R,3S,4R,5S,6R)-2-(hydroxymethyl)-6-[3-(4-pyridyloxy)phenyl]tetrahydropyran-3,4,5-triol

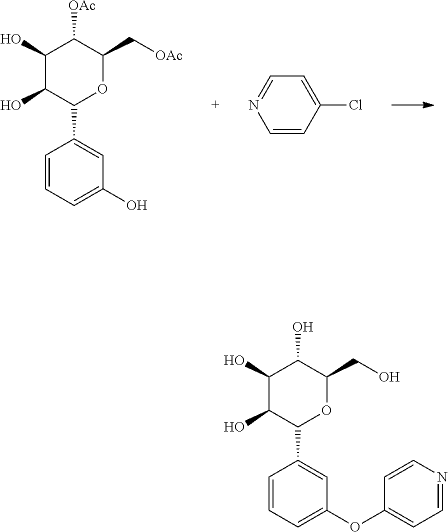

To a solution of INTERMEDIATE A (40 mg, 0.118 mmol) in DMF (800 μl) is added 4-chloropyridine (HCl salt) (21.15 mg, 0.141 mmol) followed by Cs₂CO₃ (114.9 mg, 0.353 mmol). The reaction mixture is stirred at 120° C. for 48 h, filtered and concentrated. To the resulting residue in MeOH (0.8 ml) is added 2 drops cat NaOMe (25% w/v in MeOH). After 1 h, the reaction mixture is neutralized with Amberlite IR120(H). After filtration, washing with MeOH, the solvent is removed under reduced pressure and the residue purified using reverse-phase prep-HPLC by to afford the title compound (3.4 mg, 8.2%).

¹H NMR (400 MHz, CD₃OD) δ 8.33 (s, 2H), 7.58-7.29 (m, 2H), 7.22 (s, 1H), 7.00 (d, 1H), 6.91 (s, 2H), 4.86 (d, 1H), 4.22 (dd, 1H), 3.77 (dd, 1H), 3.74-3.58 (m, 2H), 3.53 (dd, 1H), 3.44 (td, 1H). LC-MS: m/z=334.3 (M+H+).

### Preparation of Compound 253

### (2R,3S,4R,5S,6R)-2-[3-(1,3-benzothiazol-2-yloxy)phenyl]-6-(hydroxymethyl)tetrahydropyran-3,4,5-triol

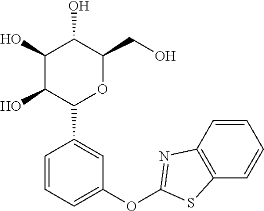

The title compound is prepared according to the procedure described for COMPOUND 243 but using INTERMEDIATE A and 2-chlorobenzo[d]thiazole.

¹H NMR (400 MHz, CD₃OD) δ 7.72 (d, 1H), 7.59 (d, 1H), 7.54-7.39 (m, 3H), 7.36 (td, 1H), 7.30-7.19 (m, 2H), 4.93 (d, 1H), 4.32-4.26 (m, 1H), 3.81 (dd, 1H), 3.77-3.64 (m, 2H), 3.58 (dd, 1H), 3.50 (td, 1H). LC-MS: m/z=390.3 (M+H+).

### Preparation of Compound 254

### [(2R,3S,4R,5S,6R)-3-acetoxy-4,5-dihydroxy-6-[3-(4-quinolyloxy)phenyl]tetrahydropyran-2-yl]methyl acetate

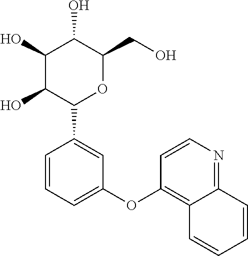

The title compound is prepared according to the procedure described for COMPOUND 243 but using INTERMEDIATE A and 4-chloroquinoline.

¹H NMR (400 MHz, CD₃OD) δ 8.54 (s, 1H), 8.32 (d, 1H), 7.93 (d, 1H), 7.84-7.67 (m, 1H), 7.63-7.52 (m, 1H), 7.38 (ddd, 3H), 7.11 (d, 1H), 6.59 (d, 1H), 4.89 (d, 1H), 4.27-4.19 (m, 1H), 3.77 (dd, 1H), 3.73-3.61 (m, 2H), 3.56 (dd, 1H), 3.46 (td, 1H). LC-MS: m/z=384.4 (M+H+).

### Preparation of Compound 255

### N-methyl-2-[3-[(2R,3S,4R,5S,6R)-3,4,5-trihydroxy-6-(hydroxymethyl)tetrahydropyran-2-yl]phenoxy]-1,3-benzothiazole-6-carboxamide

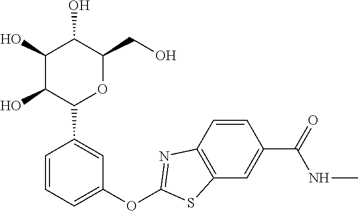

The title compound is prepared according to the procedure described for COMPOUND 243 but using INTERMEDIATE A and 2-chloro-N-methyl-1,3-benzothiazole-6-carboxamide. The later is prepared as described below.

¹H NMR (400 MHz, CD₃OD) δ 8.18 (d, 1H), 7.77 (dd, 1H), 7.61 (d, 1H), 7.51-7.38 (m, 3H), 7.24 (d, 1H), 4.90 (d, 1H), 4.31-4.14 (m, 1H), 3.87-3.60 (m, 3H), 3.54 (dd, 1H), 3.52-3.42 (m, 1H), 2.84 (s, 3H). LC-MS: m/z=447.4 (M+H+).

### Preparation of 2-chloro-N-methyl-1,3-benzothiazole-6-carboxamide

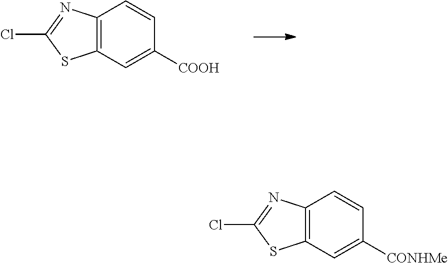

To 2-chloro-1,3-benzothiazole-6-carboxylic acid (300 mg, 1.40 mmol) in DMF (6 ml) is added a THF solution of methylamine (1.05 ml of 2M, 2.106 mmol), 2,6-lutidine (488 μl, 4.212 mmol) followed by HATU (640.7 mg, 1.685 mmol). The reaction mixture is stirred at room temperature for 3 h, diluted with H₂O, extracted with EtOAc. The organic phase is washed with sat sol NaHCO₃, brine, dried over Na₂SO₄, filtered and dried. The residue is purified on 25 g SNAP silica gel cartridge using EtOAc in Hexanes 50 to 100% to afford 2-chloro-N-methyl-1,3-benzothiazole-6-carboxamide (290 mg, 91%) as a white solid. LC-MS: m/z=227.1 (M+H+).

### Preparation of Compound 256

### (2R,3S,4R,5S,6R)-2-(hydroxymethyl)-6-(3-pyrimidin-2-yloxyphenyl)tetrahydropyran-3,4,5-triol

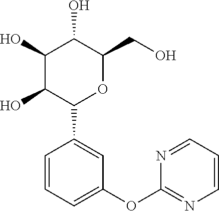

The title compound is prepared according to the procedure described for COMPOUND 243 but using INTERMEDIATE A and 2-chloropyrimidine.

¹H NMR (400 MHz, CD₃OD) δ 8.49 (d, 2H), 7.50-7.18 (m, 3H), 7.11 (t, 1H), 7.01 (d, 1H), 4.88 (d, 1H), 4.34-4.19 (m, 1H), 3.81-3.57 (m, 3H), 3.52 (dd, 1H), 3.44 (td, 1H). LC-MS: m/z=335.3 (M+H+).

### Preparation of Compound 257

### (2R,3S,4R,5S,6R)-2-(hydroxymethyl)-6-[3-[(6-nitro-3-pyridyl)oxy]phenyl]tetrahydropyran-3,4,5-triol

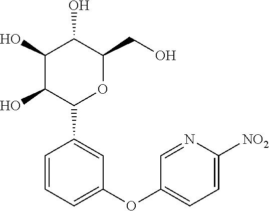

The title compound is prepared according to the procedure described for COMPOUND 243 but using INTERMEDIATE A and 5-bromo-2-nitro-pyridine as the appropriate reagent. The reaction is performed at room temperature for 20 h.

¹H NMR (400 MHz, CD₃OD) δ 8.21 (dd, 2H), 7.50 (dd, 1H), 7.47-7.38 (m, 1H), 7.34 (d, 1H), 7.27 (s, 1H), 7.04 (d, 1H), 4.85 (d, 1H), 4.27-4.14 (m, 1H), 3.77 (dd, 1H), 3.72-3.58 (m, 2H), 3.53 (dd, 1H), 3.49-3.39 (m, 1H). LC-MS: m/z=379.3 (M+H+).

### Preparation of Compound 258

### (2R,3S,4R,5S,6R)-2-[3-[(6-amino-3-pyridyl)oxy]phenyl]-6-(hydroxymethyl)tetrahydropyran-3,4,5-triol

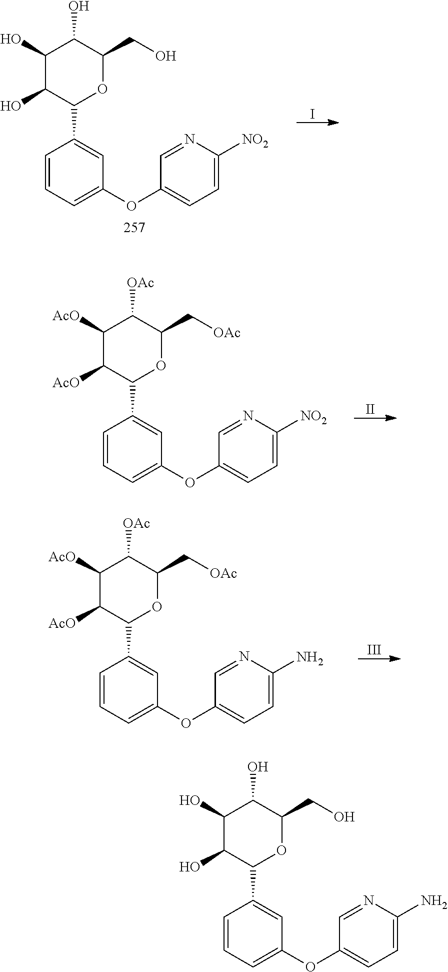

### Step I: [(2R,3R,4R,5R,6R)-3,4,5-triacetoxy-6-[3-[(6-nitro-3-pyridyl)oxy]phenyl]tetrahydropyran-2-yl]methyl acetate

To a solution of COMPOUND 257 (300 mg, 0.793 mmol) in pyridine (3 ml) is added acetic anhydride (748.2 μl, 7.93 mmol) followed by catalytic DMAP (4.85 mg, 0.04 mmol). The reaction mixture is stirred at room temperature overnight, concentrated to dryness, diluted with EtOAc. The organic phase is carefully washed with sat sol NaHCO₃, H₂O, brine then dried over Na₂SO₄, filtered and dried. The residue is purified on 25 g SNAP silica gel cartridge using EtOAc in Hexanes 5 to 100% to afford the title compound (180 mg, 41.5%). LC-MS: m/z=547.4 (M+H+).

### Step II: [(2R,3R,4R,5R,6R)-3,4,5-triacetoxy-6-[3-[(6-amino-3-pyridyl)oxy]phenyl]tetrahydropyran-2-yl]methyl acetate

To [(2R,3R,4R,5R,6R)-3,4,5-triacetoxy-6-[3-[(6-nitro-3-pyridyl)oxy]phenyl]tetrahydropyran-2-yl]methyl acetate (130 mg, 0.238 mmol) in MeOH (1.95 ml) is added 10% Pd on Charcoal (50.63 mg, 0.0476 mmol). The reaction mixture is hydrogenated under 1 atm of H₂ overnight. The catalyst is filtered on celite, washed with MeOH. The filtrate is concentrated and the residue purified on 10 g SNAP silica gel cartridge using EtOAc in Hexanes 30 to 100% to afford a mixture. The title compound is the major constituent of this mixture and is used as such in the next step without further purification. LC-MS: m/z=517.5 (M+H+).

### Step III: Compound 258

To the previous residue (35 mg) in MeOH (650 μl) is added 2 drops of catalytic NaOMe (25% w/v in MeOH). The reaction mixture is stirred at room temperature for 1 h, neutralized with Amberlite IR120(H). After filtration, the solvent is removed under reduced pressure and the residue purified using reverse-phase prep-HPLC to afford the title compound (11.4 mg, 13.5%).

¹H NMR (400 MHz, CD₃OD) δ 7.60 (s, 1H), 7.32-7.13 (m, 2H), 7.07 (d, 1H), 6.99 (s, 1H), 6.75 (dd, 1H), 6.56 (d, 1H), 4.82 (d, J=4.0 Hz, 1H), 4.24 (t, 1H), 3.78-3.57 (m, 3H), 3.47 (dd, 1H), 3.41-3.29 (m, 1H). LC-MS: m/z=349.3 (M+H+).

### Preparation of Compound 259

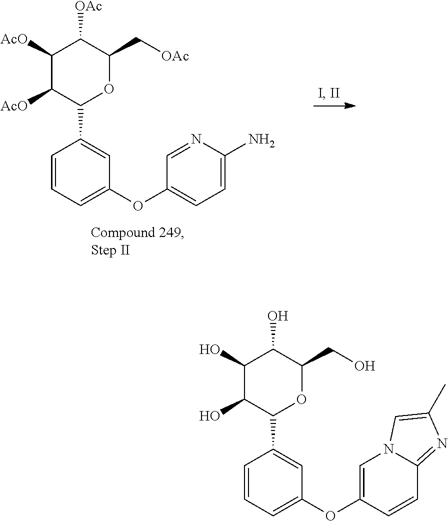

### Step I: [(2R,3R,4R,5R,6R)-3,4,5-triacetoxy-6-[3-(2-methylimidazo[1,2-a]pyridin-6-yl)oxyphenyl]tetrahydropyran-2-yl]methyl acetate

To a solution of [(2R,3R,4R,5R,6R)-3,4,5-triacetoxy-6-[3-[(6-amino-3-pyridyl)oxy]phenyl]tetrahydropyran-2-yl]methyl acetate (COMPOUND 249, Step II) (50 mg, 0.097 mmol) in DMF (1 ml) is added chloroacetone (11.56 μl, 0.145 mmol). The reaction mixture is stirred at 70° C. overnight, then another 1.5 eq of chloroacetone (11.56 μl, 0.1452 mmol) is added and the reaction mixture is stirred for 5 h at 70° C. The reaction mixture is concentrated to dryness to afford the title compound which is used in the next step without further purification. LC-MS: m/z=555.5 (M+H+).

### Step II: Compound 259

To the previous crude (53 mg) in MeOH (1 ml) is added 2 drops catalytic NaOMe (25% w/v in MeOH). The reaction mixture is stirred for 2 h at room temperature, diluted with MeOH, neutralized with Amberlite IR120(H). After filtration, washing with MeOH, the solvent is removed under reduced pressure and the residue purified by reverse-phase prep-HPLC to afford the title compound (3 mg, 7.3%).

¹H NMR (400 MHz, CD₃OD) δ 8.14 (s, 1H), 7.39 (s, 2H), 7.29 (t, 1H), 7.22-6.98 (m, 3H), 6.88 (dd, 1H), 4.82 (d, 1H), 4.28-4.08 (m, 1H), 3.83-3.56 (m, 3H), 3.49 (dd, 1H), 3.44-3.25 (m, 1H), 2.30 (s, 3H). LC-MS: m/z=387.4 (M+H+).

### Preparation of Compound 260

### (2R,3S,4R,5S,6R)-2-(hydroxymethyl)-6-[3-(hydroxymethyl)phenyl]tetrahydropyran-3,4,5-triol

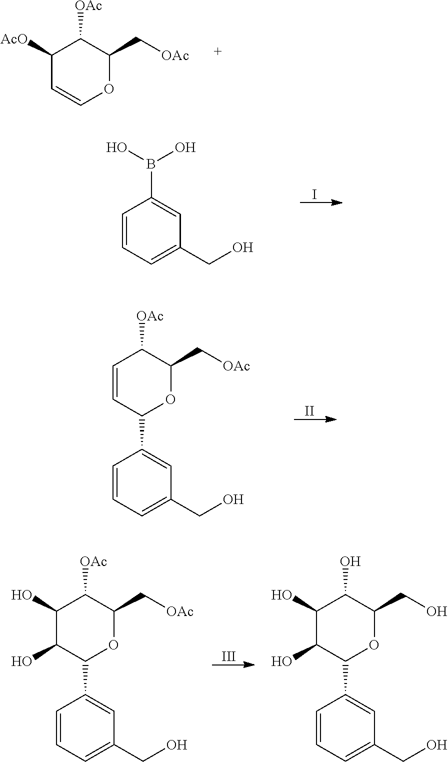

### Step I: [(2R,3S,6S)-3-acetoxy-6-[3-(hydroxymethyl)phenyl]-3,6-dihydro-2H-pyran-2-yl]methyl acetate

To a solution of [(2R,3S,4R)-3,4-diacetoxy-3,4-dihydro-2H-pyran-2-yl]methyl acetate (500 mg, 1.837 mmol) and [3-(hydroxymethyl)phenyl]boronic acid (558.3 mg, 3.674 mmol) in acetonitrile (5 ml) is added Pd(OAc)₂ (61.87 mg, 0.276 mmol). The reaction mixture is stirred at room temperature for 5 h. 1 eq [3-(hydroxymethyl)phenyl]boronic acid and 0.075 eq Pd(OAc)₂ are added and the reaction mixture is stirred at room temperature overnight. The mixture is diluted with CH₂Cl₂ and filtered through a pad of celite. The filtrate is concentrated and the residue purified on 25 g SNAP silica gel cartridge using EtOAc in Hexanes 10 to 60% to afford the title compound. (320 mg, 54.4%) as a colorless oil. LC-MS: m/z=343.3 (M+Na⁺).

### Step II: [(2R,3S,4R,5S,6R)-3-acetoxy-4,5-dihydroxy-6-[3-(hydroxymethyl)phenyl]tetrahydropyran-2-yl]methyl acetate

To a solution of [(2R,3S,65)-3-acetoxy-6-[3-(hydroxymethyl)phenyl]-3,6-dihydro-2H-pyran-2-yl]methyl acetate (135 mg, 0.4214 mmol) in a mixture water (540 μl)/t-BuOH (540 μl) are added methanesulfonamide (60.13 mg, 0.632 mmol), 2.5% OsO4/t-BuOH (214.3 μl of 2.5% w/v, 0.0210 mmol), NMO (98.73 mg, 0.843 mmol) and 2,6-lutidine (48.81 μl, 0.4214 mmol). The mixture is stir red at room temperature overnight, quenched with 15% sodium bisulfite and diluted with ethyl acetate. The aqueous phase is separated, washed with water and brine, dried over sodium sulfate. After removal of the solvent under reduced pressure, the residue is purified on 25 g SNAP silica gel cartridge using MeOH in CH₂Cl₂ 0 to 12% to afford the title compound (48 mg, 32%) as a white solid. LC-MS: m/z=377.3 (M+Na⁺).

### Step III: Compound 260

To a solution of the previous residue (48 mg, 0.135 mmol) in MeOH (540 μL) is added 2 drops of NaOMe (25% w/v in MeOH). The reaction mixture is stirred at room temperature for 2 h, diluted with MeOH, neutralized with Amberlite IR120(H). After filtration, washing with MeOH, the solvent is removed under reduced pressure and the residue purified by reverse-phase prep-HPLC to afford the title compound (21.5 mg, 18%).

¹H NMR (400 MHz, CD₃OD) δ 7.45 (s, 1H), 7.35 (dt, 2H), 7.26 (d, 1H), 4.96 (d, 1H), 4.60 (s, 2H), 4.44 (t, 1H), 3.86-3.77 (m, 2H), 3.73 (t, 1H), 3.57 (dd, 1H), 3.51-3.39 (m, 1H). LC-MS: m/z=293.3 (M+Na⁺).

### Preparation of Compound 261

### methyl 4-[[3-[(2R,3S,4R,5S,6R)-3,4,5-trihydroxy-6-(hydroxymethyl)tetrahydropyran-2-yl]phenyl]methoxy]benzoate

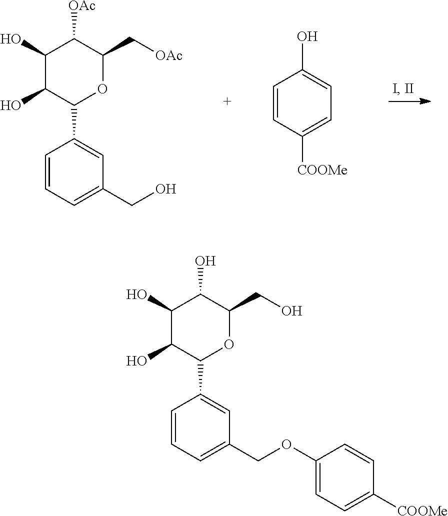

### Step I: methyl 4-[[3-[(2R,3S,4R,5S,6R)-5-acetoxy-6-(acetoxymethyl)-3,4-dihydroxy-tetrahydropyran-2-yl]phenyl]methoxy]benzoate

To methyl 4-hydroxybenzoate (34.35 mg, 0.226 mmol) in THF (1.6 ml) is added [(2R,3S,4R,5S,6R)-3-acetoxy-4,5-dihydroxy-6-[3-(hydroxymethyl)phenyl]tetrahydropyran-2-yl]methyl acetate from COMPOUND 251, Step II (80 mg, 0.226 mmol), PPh₃ (62.19 mg, 0.237 mmol) followed by DIAD (45.92 μl, 0.237 mmol) dropwise. The reaction mixture is stirred at room temperature overnight, concentrated and purified on 10 g SNAP silica gel cartridge using EtOAc in Hexanes 5 to 100% as eluent to afford the title compound as a mixture which is used in the next step without further purification. LC-MS: m/z=511.4 (M+Na⁺).

### Step II: Compound 261

To the previous residue (70 mg, 0.14 mmol) in MeOH (800 μl) is added 2 drops of NaOMe (25% w/v in MeOH). The reaction is stirred at room temperature overnight, neutralized with Amberlite IR120(H). After filtration, washing with MeOH, the solvent is removed under reduced pressure and the residue purified by reverse-phase prep-HPLC to afford the title compound (20.2 mg, 21.5%).

¹H NMR (400 MHz, CD₃OD) δ 7.91-7.70 (m, 2H), 7.50 (s, 1H), 7.42-7.15 (m, 3H), 7.06-6.83 (m, 2H), 5.09 (s, 2H), 4.87 (t, 1H), 4.33 (t, 1H), 3.83-3.73 (m, 3H), 3.72 (t, 2H), 3.69-3.58 (m, 1H), 3.49 (dd, 1H), 3.43-3.32 (m, 1H). LC-MS: m/z=427.4 (M+Na⁺).

### Preparation of Compound 262

### (2R,3S,4R,5S,6R)-2-(hydroxymethyl)-6-[3-(phenoxymethyl)phenyl]tetrahydropyran-3,4,5-triol

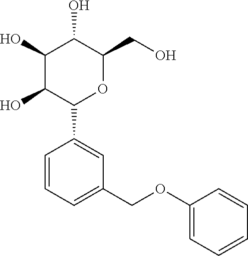

The title compound is prepared according to the procedure described for COMPOUND 261 but using phenol as reagent.

¹H NMR (400 MHz, CD₃OD) δ 7.48 (s, 1H), 7.31 (dq, 3H), 7.16 (dd, 2H), 6.96-6.68 (m, 3H), 5.00 (s, 2H), 4.89 (d, 1H), 4.35 (t, 1H), 3.78-3.60 (m, 3H), 3.49 (dd, 1H), 3.43-3.30 (m, 1H). LC-MS: m/z=369.3 (M+Na⁺).

### Preparation of Compound 263

### (2R,3S,4R,5S,6R)-2-[3-[(3,5-dichlorophenoxy)methyl]phenyl]-6-(hydroxymethyl)tetrahydropyran-3,4,5-triol

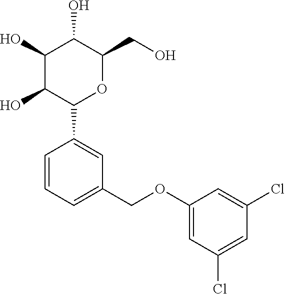

The title compound is prepared according to the procedure described for COMPOUND 261 but using 3,5-dichlorophenol as reagent.

¹H NMR (400 MHz, CD₃OD) δ 7.56 (s, 1H), 7.40 (dq, 3H), 6.99 (s, 3H), 5.10 (s, 2H), 4.95 (t, 1H), 4.41 (t, 1H), 3.90-3.77 (m, 2H), 3.74 (dd, 1H), 3.57 (dd, 1H), 3.46 (td, 1H). LC-MS: m/z=437.3 (M+Na⁺).

### Preparation of Compound 264

### N-methyl-4-[[3-[(2R,3S,4R,5S,6R)-3,4,5-trihydroxy-6-(hydroxymethyl)tetrahydropyran-2-yl]phenyl]methoxy]benzamide

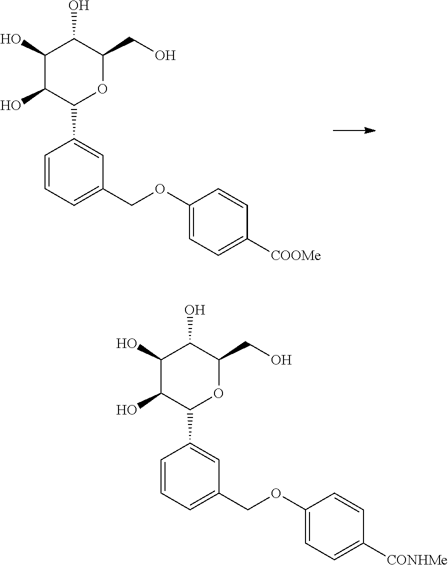

To COMPOUND 261 in a mixture 3/2/1 THF (75 μl)/MeOH (50 μl)/H₂O (25 μl) is added LiOH (2.7 mg, 0.113 mmol). The reaction mixture is stirred at room temperature overnight, acidified by a 3N HCl solution, concentrated to dryness. To the previous residue (14.7 mg) in DMF (306 μl) is added methylamine (28.33 μL of 2 M solution in MeOH, 0.0566 mmol), 2,6-lutidine (13.12 μl, 0.113 mmol) followed by HATU (17.23 mg, 0.0453 mmol). The reaction mixture is stirred at room temperature overnight, concentrated and purified by reverse-phase prep-HPLC to afford the title compound (8.7 mg, 54.8%).

¹H NMR (400 MHz, CD₃OD) δ 7.72-7.59 (m, 2H), 7.49 (s, 1H), 7.42-7.18 (m, 3H), 6.95 (d, 2H), 5.07 (s, 2H), 4.87 (t, 1H), 4.33 (t, 1H), 3.79-3.59 (m, 3H), 3.57-3.43 (m, 1H), 3.41-3.30 (m, 1H), 2.76 (d, 3H). LC-MS: m/z=404.3 (M+H⁺).

### Preparation of Compound 265

### (2R,3S,4S,5S,6R)-2-[(2R,3R,4R,5R,6R)-3,5-Dihydroxy-2-(hydroxymethyl)-6-(3-hydroxyphenyl)tetrahydropyran-4-yl]oxy-6-(hydroxymethyl)tetrahydropyran-3,4,5-triol

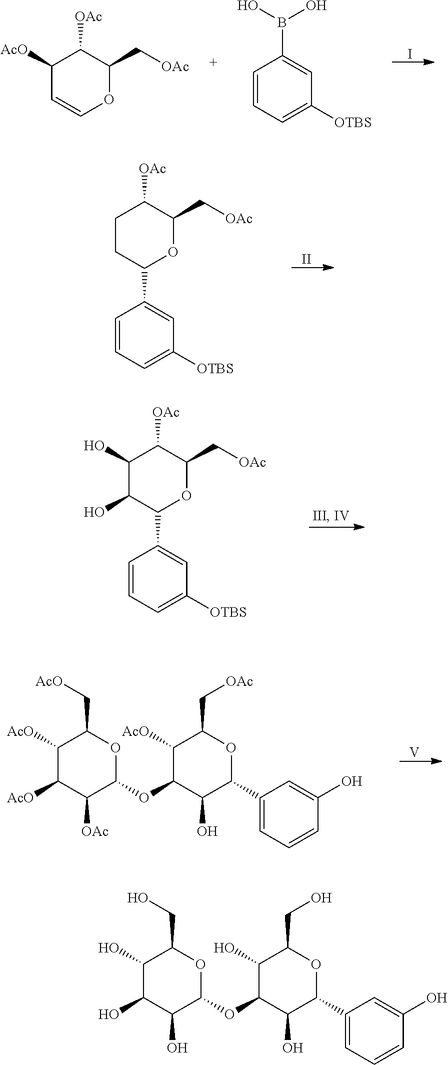

### Step I: [(2R,3S,6S)-3-Acetoxy-6-[3-[tert-butyl(dimethyl)silyl]oxyphenyl]-3,6-dihydro-2H-pyran-2-yl]methyl acetate

To a solution of [(2R,3S,4R)-3,4-diacetoxy-3,4-dihydro-2H-pyran-2-yl]methyl acetate (1100 mg, 4.040 mmol) in 10 mL of acetonitrile are added [3-(tert-butyl-dimethyl-silyl)oxyphenyl]boronic acid (2.038 g, 8.080 mmol) and Pd(OAc)₂ (136.1 mg, 0.6060 mmol). The mixture is stirred at rt for 5 h and then to it are added another batch of Pd(OAc)₂ (136.1 mg, 0.606 mmol) and [3-(tert-butyl-dimethyl-silyl)oxyphenyl]boronic acid (2.038 g, 8.080 mmol). It is then stirred at rt overnight. The mixture is diluted with 20 mL of DCM and filtered over a pad of celite. The filtrate is concentrated and the residue is separated on Biotage SNAP 100 g silica gel cartridge using a gradient of ethyl acetate/hexane (0-20% in 20 CV) to obtain [(2R,3S,65)-3-acetoxy-6-[3-[tert-butyl(dimethyl)silyl]oxyphenyl]-3,6-dihydro-2H-pyran-2-yl]methyl acetate (805 mg, 47.38%) as an oil, which solidifies upon standing.

¹H NMR (CDCl₃, 400 MHz): 7.06 (m, 1H), 6.78 (m, 1H), 6.70 (m, 1H), 6.60 (m, 1H), 5.97 (m, 1H), 5.71 (m, 1H), 5.09 (m, 2H), 4.08 (m, 1H), 3.85 (m, 1H), 3.62 (m, 1H), 1.88 and 1.87 (2s, 6H), 0.78 (m, 9H), 0.00 (m, 6H).

### Step II: [(2R,3S,4R,5S,6R)-3-Acetoxy-6-[3-[tert-butyl(dimethyl)silyl]oxyphenyl]-4,5-dihydroxy-tetrahydropyran-2-yl]methyl acetate

To a solution of [(2R,3S,6S)-3-acetoxy-6-[3-[tert-butyl(dimethyl)silyl]oxyphenyl]-3,6-dihydro-2H-pyran-2-yl]methyl acetate (2.5 g, 5.944 mmol) in water (10 mL)/t-BuOH (10 mL) are added methanesulfonamide (848.1 mg, 8.916 mmol), 2.5% OsO₄/t-BuOH (1.865 mL, 0.1486 mmol), NMO (1.393 g, 11.89 mmol) and 2,6-lutidine (636.9 mg, 688.5 μL, 5.944 mmol). The mixture is stirred at RT overnight. It is then quenched with 15% sodium bisulfate (15 mL) and diluted with ethyl acetate (40 mL). The aqueous phase is separated, and the organic layer is washed with water (20 mL) and brine (20 mL) consecutively, dried over sodium sulfate. After removal of the solvent under reduced pressure, the residue is purified on Biotage SNAP 100 g silica gel cartridge using a gradient of MeOH/DCM (0-6% in 20 CV) to obtain [(2R,3S,4R,5S,6R)-3-acetoxy-6-[3-[tert-butyl(dimethyl)silyl]oxyphenyl]-4,5-dihydroxy-tetrahydropyran-2-yl]methyl acetate (2.2 g, 81.42%) as an oil.

1H NMR (CD₃OD, 400 MHz): 7.06 (m, 1H), 6.78 (m, 1H), 6.70 (m, 1H), 6.58 (m, 1H), 4.85 (m, 1H), 4.64 (m, 1H), 4.46 (m, 1H), 3.96 (m, 1H), 3.85 (m, 1H), 3.62 (m, 2H), 1.86 and 1.83 (2s, 6H), 0.78 (m, 9H), 0.00 (m, 6H).

### Step III: [(2R,3R,4R,5R,6R)-3-Acetoxy-6-[3-[tert-butyl(dimethyl)silyl]oxyphenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate

To a stirred solution of [(2R,3R,4S,5S,6R)-3,4,5-triacetoxy-6-(2,2,2-trichloroethanimidoyl)oxy-tetrahydropyran-2-yl]methyl acetate (300 mg, 0.5176 mmol) and [(2R,3S,4R,5S,6R)-3-acetoxy-6-[3-[tert-butyl(dimethyl)silyl]oxyphenyl]-4,5-dihydroxy-tetrahydropyran-2-yl]methyl acetate (258.8 mg, 0.5694 mmol) in DCM (10 mL) is added 4 A MS (1.0 g), stirred at rt for 30 min. After cooling to −40° C., freshly opened trimethylsilyl trifluoromethanesulfonate (9.4 μL, 0.052 mmol) is added dropwise. The mixture is stirred at −40° C. and slowly warmed up to −10° C. in 2 h. Then Et₃N (72.2 μL, 0.5180 mmol) is added. After removal of the cooling bath, the mixture is warmed to rt, filtered off to remove the molecular sieves, and concentrated to dryness. The residue is purified on Biotage SNAP 50 g silica gel cartridge using MeOH/DCM (0 to 5% in 20 CV) to provide an inseparable mixture containing [(2R,3R,4R,5R,6R)-3-acetoxy-6-[3-[tert-butyl(dimethyl)silyl]oxyphenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate (375 mg), which is used directly in the next step without further purification.

LC-MS: m/z=807.5 (M+Na⁺).

### Step IV: [(2R,3R,4R,5R,6R)-3-Acetoxy-5-hydroxy-6-(3-hydroxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate

To a stirred solution of the mixture containing [(2R,3R,4R,5R,6R)-3-acetoxy-6-[3-[tert-butyl(dimethyl)silyl]oxyphenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate (375 mg, 0.4778 mmol) in THF (4 mL) are added acetic acid (40.8 μL, 0.7174 mmol) and 1M TBAF/THF (1.433 mL, 1.433 mmol). The mixture is stirred at rt for 30 min. It is then diluted with ethyl acetate (30 mL), washed with water (20 mL) and brine (20 mL) consecutively, dried over sodium sulfate, concentrated to dryness. The residue is purified on Biotage SNAP 25 g silica gel cartridge using MeOH/DCM (0-6% in 20 CV) to obtain an inseparable mixture (230 mg), containing [(2R,3R,4R,5R,6R)-3-acetoxy-5-hydroxy-6-(3-hydroxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate. It is used directly in the next step without further purification.

LC-MS: m/z=693.3 (M+Na⁺).

### Step V: Compound 265

To a stirred solution of a mixture containing [(2R,3R,4R,5R,6R)-3-acetoxy-5-hydroxy-6-(3-hydroxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate (66 mg, 0.09842 mmol) in MeOH (3 mL) is added 25% MeONa (2.0 μL, 0.01 mmol) and stirred at rt overnight. It is then neutralized with resin Amberlite IR120 (H) and filtered. The filtrate is concentrated to dryness under reduced pressure. The residue is purified using reverse-phase prep HPLC to provide (2R,3S,4S,5S,6R)-2-[(2R,3R,4R,5R,6R)-3,5-dihydroxy-2-(hydroxymethyl)-6-(3-hydroxyphenyl)tetrahydropyran-4-yl]oxy-6-(hydroxymethyl)tetrahydropyran-3,4,5-triol (27 mg) as a white solid.

¹H NMR (CD₃OD, 400 MHz): 7.09 (m, 1H), 6.83 (m, 2H), 6.57 (m, 1H), 5.08 (d, 1H), 5.04 (d, 1H), 4.42 (m, 1H), 3.90 (m, 1H), 3.58-3.77 (m, 5H), 3.47-3.54 (m, 3H), 3.40 (m, 2H). LC-MS: m/z=418.4 (M+H⁺).

### Preparation of Compound 266

### (2R,3S,4S,5S,6R)-2-[(2R,3R,4R,5R,6R)-3,5-Dihydroxy-2-(hydroxymethyl)-6-(4-hydroxyphenyl)tetrahydropyran-4-yl]oxy-6-(hydroxymethyl)tetrahydropyran-3,4,5-triol

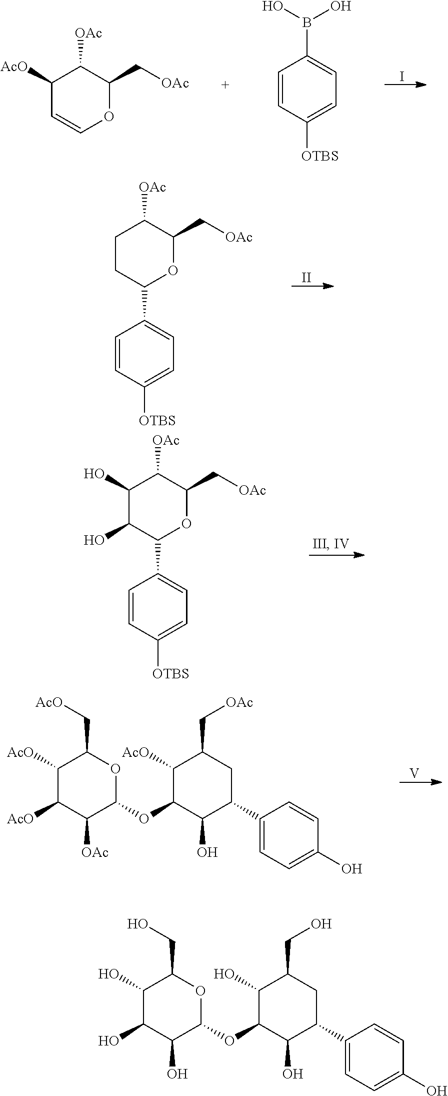

Step I and II are carried out using a similar procedure for COMPOUND 265.

### Step III

To a stirred solution of [(2R,3R,4S,5S,6R)-3,4,5-triacetoxy-6-(2,2,2-trichloroethanimidoyl)oxy-tetrahydropyran-2-yl]methyl acetate (486.9 mg, 0.8400 mmol) and [(2R,3S,4R,5S,6R)-3-acetoxy-6-[4-[tert-butyl(dimethyl)silyl]oxyphenyl]-4,5-dihydroxy-tetrahydropyran-2-yl]methyl acetate (381.9 mg, 0.8400 mmol) in DCM (15 mL) is added 4 A MS (1.0 g) and stirred at rt for 30 min. After cooling to −40° C., trimethylsilyl trifluoromethanesulfonate (7.6 μL, 0.04206 mmol) is added dropwise, and the mixture is stirred at −40° C. under nitrogen and slowly warmed up to −10° C. in 1 h. Then Et₃N (117.1 μL, 0.8400 mmol) is added and it is warmed to rt. After filtration to remove the molecular sieves, the solvent is evaporated under reduced pressure, and the residue is purified on Biotage SNAP 50 g silica gel cartridge using MeOH/DCM (0 to 5% in 20 CV) to provide an inseparable mixture (700 mg) containing several fractions with the desired mass, which is used in the next step without further purification.

LC-MS: m/z=807.5 (M+Na⁺).

### Step IV: [(2R,3R,4R,5R,6R)-3-Acetoxy-5-hydroxy-6-(4-hydroxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate

To the above-mentioned mixture (700 mg) in 10 mL of THF are added acetic acid (47.8 μL, 0.8405 mmol) and 1M TBAF/THF (1.680 mL, 1.680 mmol). The mixture is stirred at rt for 1 h. After removal of the solvent under reduced pressure, the residue is dissolved in DCM (20 mL), washed with water and brine consecutively, dried over sodium sulfate, and concentrated to dryness. The residue is purified on Biotage SNAP 50 g silica gel cartridge using MeOH/DCM (0-5% in 20 CV) to provide an inseparable mixture (300 mg) containing the title compound, which is used in the next step without further purification.

LC-MS: m/z=693.3 (M+Na⁺).

### Step V: Compound 266

To a solution of the above-mentioned mixture (50 mg) in methanol (3 mL) is added a drop of 25% sodium methoxide/methanol. After stirring for 20 min at rt, it is neutralized with Amberlite IR120 (H). After filtration, the solvent is removed under reduced pressure and the residue is purified using reverse-phase prep-HPLC to obtain the title compound (9 mg) as a white solid.

1H NMR (400 MHz, CD₃OD): δ 7.19 (d, 2H), 6.68 (d, 2H), 5.04 (m, 2H), 4.41 (s, 1H), 3.89 (m, 1H), 3.77-3.57 (m, 6H), 3.56-3.44 (m, 2H), 3.38 (m, 2H). LC-MS: m/z=441.3 (M+Na⁺).

### Preparation of Compound 267

### 4-[3-[(2R,3R,4R,5R,6R)-3,5-Dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenoxy]-N-methyl-benzamide

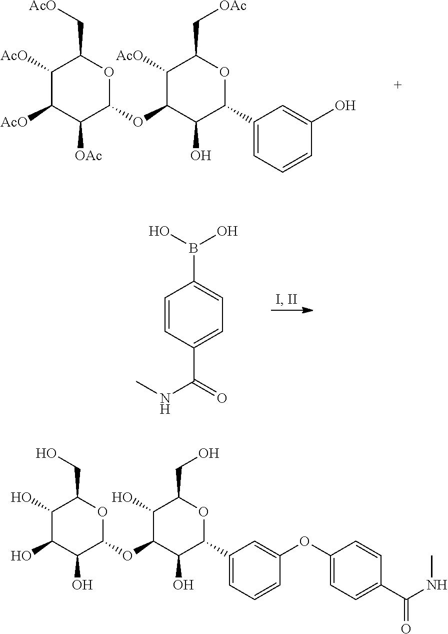

To a solution of [(2R,3R,4R,5R,6R)-3-acetoxy-5-hydroxy-6-(3-hydroxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate (236 mg, 0.3519 mmol) in 5 mL of DCM are added (4-N-methylaminocarbonylphenyl)boronic acid (126.6 mg, 0.7034 mmol), molecular sieves 4 Å (500 mg) and Cu(OAc)₂ (89.49 mg, 0.4927 mmol). After stirring for 10 min, 2,6-lutidine (203.9 μL, 1.760 mmol) is added to the mixture. It is stirred at rt for 2 days. After removal of the solvent under reduced pressure, the residue is separated on Biotage SNAP 25 g silica gel cartridge using a gradient of MeOH/DCM (0-6% in 20 CV) to obtain a mixture containing the desired product (about 240 mg), which is used in the next step without further purification. To a solution of the above-mentioned mixture (240 mg) in methanol (3 mL) is added a drop of 25% MeONa/MeOH. After stirring for 20 min at rt, it is neutralized with Amberlite IR120 (H). After filtration, the solvent is removed under reduced pressure and the residue is purified using reverse-phase prep-HPLC to obtain the title compound (55 mg) as a white solid.

¹H NMR (CD₃OD, 400 MHz): 7.80 (m, 2H), 7.41 (m, 1H), 7.31 (m, 1H), 7.20 (s, 1H), 7.02 (m, 2H), 6.96 (m, 1H), 5.18 (d, 1H), 5.09 (d, 1H), 4.42 (m, 1H), 3.95 (m, 1H), 3.64-3.77 (m, 6H), 3.54 (m, 3H), 3.32 (m, 1H), 2.89 (s, 3H).

LC-MS: m/z=552.3 (M+H⁺);

### Preparation of Compound 268

### 3-[4-[(2R,3R,4R,5R,6R)-3,5-Dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenoxy]-N-methyl-benzamide

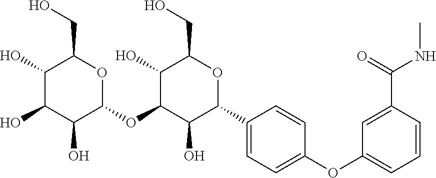 [SMILES: CNC(=O)c1cccc(Oc2ccc([C@H]3O[C@H](CO)[C@@H](O)[C@H](O[C@H]4O[C@H](CO)[C@@H](O)[C@H](O)[C@@H]4O)[C@@H]3O)cc2)c1]

The title compound is prepared as described in COMPOUND 267 but using (3-N-methylaminocarbonylphenyl)boronic acid as reagent.

¹H NMR (400 MHz, CD₃OD): δ 7.39 (m, 5H), 7.08 (m, 1H), 6.92 (d, 2H), 5.06 (d, 1H), 5.02 (s, 1H), 4.37 (m, 1H), 3.90-3.84 (m, 1H), 3.83-3.65 (m, 4H), 3.65-3.41 (m, 6H), 2.79 (s, 3H). LC-MS: m/z=552.3 (M+H⁺).

### Preparation of Compound 269

### 3-[3-[(2R,3R,4R,5R,6R)-3,5-Dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenoxy]-N-methyl-benzamide

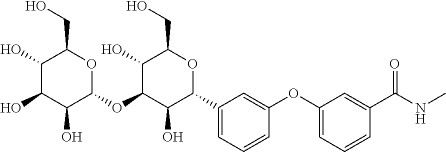 [SMILES: CNC(=O)c1cccc(Oc2cccc([C@H]3O[C@H](CO)[C@@H](O)[C@H](O[C@H]4O[C@H](CO)[C@@H](O)[C@H](O)[C@@H]4O)[C@@H]3O)c2)c1]

The title compound is prepared as described in COMPOUND 267 but using (3-N-methylaminocarbonylphenyl)boronic acid as reagent.

¹H NMR (400 MHz, CD₃OD): δ 7.45 (d, 1H), 7.33 (m, 3H), 7.18 (m, 1H), 7.08 (m, 2H), 6.83 (d, 1H), 5.25-4.91 (m, 2H), 4.38 (s, 1H), 3.87 (m, 1H), 3.70 (m, 2H), 3.66-3.53 (m, 4H), 3.54-3.35 (m, 3H), 3.32-3.24 (m, 1H), 3.03 (d, 3H).

LC-MS: m/z=552.3 (M+H⁺).

### Preparation of Compound 270

### (2R,3S,4S,5S,6R)-2-[(2R,3R,4R,5R,6R)-2-[3-(4-Fluorophenoxy)phenyl]-3,5-dihydroxy-6-(hydroxymethyl)tetrahydropyran-4-yl]oxy-6-(hydroxymethyl)tetrahydropyran-3,4,5-triol 4.3 mg White Powder, 16.7% Yield

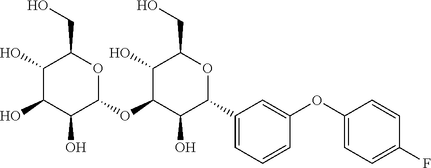 [SMILES: OC[C@H]1O[C@H](O[C@@H]2[C@H](O)[C@@H](c3cccc(Oc4ccc(F)cc4)c3)O[C@H](CO)[C@H]2O)[C@@H](O)[C@@H](O)[C@@H]1O]

The title compound is prepared as described in COMPOUND 267 but using 4-fluorophenylboronic acid as reagent.

¹H NMR (400 MHz, CD₃OD): δ 7.25 (m, 1H), 7.13 (d, 1H), 7.05-6.87 (m, 5H), 6.76 (m, 1H), 5.03 (m, 2H), 4.36 (m, 1H), 3.86 (m, 1H), 3.7-3.4 (m, 9H), 3.21 (m, 1H). LC-MS: m/z=513.2 (M+H⁺).

### Preparation of Compound 271

### (2R,3S,4S,5S,6R)-2-[(2R,3R,4R,5R,6R)-3,5-Dihydroxy-2-(hydroxymethyl)-6-(3-phenoxyphenyl)tetrahydropyran-4-yl]oxy-6-(hydroxymethyl)tetrahydropyran-3,4,5-triol 4.2 mg White Powder, 15.5% Yield

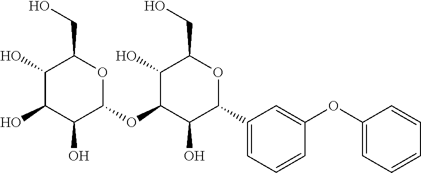 [SMILES: OC[C@H]1O[C@H](O[C@@H]2[C@H](O)[C@@H](c3cccc(Oc4ccccc4)c3)O[C@H](CO)[C@H]2O)[C@@H](O)[C@@H](O)[C@@H]1O]

The title compound is prepared as described in COMPOUND 267 but using phenylboronic acid as reagent.

¹H NMR (400 MHz, CD₃OD): δ 7.26 (m, 2H), 7.14 (d, 1H), 7.02 (m, 2H), 6.90 (m, 2H), 6.78 (m, 2H), 5.10 (d, 1H), 5.01 (d, 1H), 4.60-4.16 (m, 1H), 3.87 (dd, 1H), 3.76-3.44 (m, 4H), 3.44-3.35 (m, 2H), 3.49-3.29 (m, 4H). LC-MS: m/z=495.2 (M+H⁺).

### Preparation of Compound 272

### (2R,3S,4S,5S,6R)-2-[(2R,3R,4R,5R,6R)-3,5-Dihydroxy-2-(hydroxymethyl)-6-[3-[4-(5-methyl-1,3,4-oxadiazol-2-yl)phenoxy]phenyl]tetrahydropyran-4-yl]oxy-6-(hydroxymethyl)tetrahydropyran-3,4,5-triol

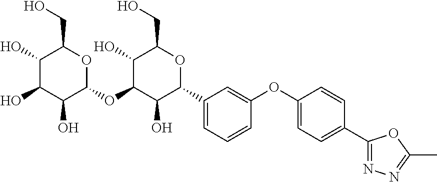 [SMILES: Cc1nnc(-c2ccc(Oc3cccc([C@H]4O[C@H](CO)[C@@H](O)[C@H](O[C@H]5O[C@H](CO)[C@@H](O)[C@H](O)[C@@H]5O)[C@@H]4O)c3)cc2)o1]

The title compound is prepared as described in COMPOUND 267 but using (4-(5-methyl-1,3,4-oxadiazol-2-yl)phenyl)boronic acid as reagent.

¹H NMR (400 MHz, CD₃OD): δ 7.97-7.84 (m, 2H), 7.35 (m, 1H), 7.25 (d, 1H), 7.17 (s, 1H), 7.11-7.00 (m, 2H), 6.92 (d, 1H), 5.06 (m, 2H), 4.43 (m, 1H), 3.87 (m, 1H), 3.7-3.22 (m, 10H), 2.51 (s, 3H). LC-MS: m/z=577.3 (M+H⁺).

### Preparation of Compound 273

### N-[4-[3-[(2R,3R,4R,5R,6R)-3,5-Dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenoxy]phenyl]-2-methyl-propanamide

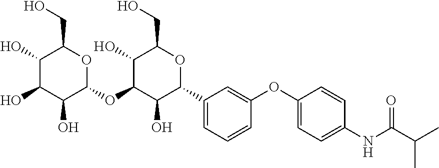 [SMILES: CC(C)C(=O)Nc1ccc(Oc2cccc([C@H]3O[C@H](CO)[C@@H](O)[C@H](O[C@H]4O[C@H](CO)[C@@H](O)[C@H](O)[C@@H]4O)[C@@H]3O)c2)cc1]

The title compound is prepared as described in COMPOUND 267 but using (4-isobutyramidophenyl)boronic acid as reagent.

¹H NMR (400 MHz, CD₃OD): δ 9.63 (s, 1H), 7.44 (m, 2H), 7.24 (m, 1H), 7.12 (d, 1H), 7.03 (s, 1H), 6.94-6.82 (m, 2H), 6.77 (d, 1H), 5.04 (m, 2H), 4.37 (m, 1H), 3.87 (m, 1H), 3.79-3.55 (m, 6H), 3.57-3.44 (m, 2H), 3.40 (dd, 1H), 3.35-3.25 (m, 1H), 2.52 (dt, 1H), 1.10 (d, 6H). LC-MS: m/z=580.3 (M+H⁺).

### Preparation of Compound 274

### (2R,3S,4S,5S,6R)-2-[(2R,3R,4R,5R,6R)-2-[3-(3,5-Dimethylphenoxy)phenyl]-3,5-dihydroxy-6-(hydroxymethyl)tetrahydropyran-4-yl]oxy-6-(hydroxymethyl)tetrahydropyran-3,4,5-triol

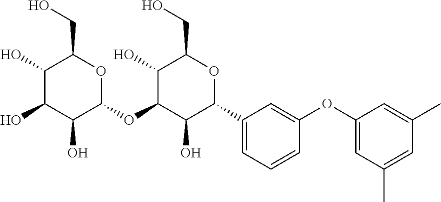 [SMILES: Cc1cc(C)cc(Oc2cccc([C@H]3O[C@H](CO)[C@@H](O)[C@H](O[C@H]4O[C@H](CO)[C@@H](O)[C@H](O)[C@@H]4O)[C@@H]3O)c2)c1]

The title compound is prepared as described in COMPOUND 267 but using 3-5-dimethylphenyl boronic acid as reagent

¹H NMR (400 MHz, CD₃OD): δ 7.31 (m, 1H), 7.18 (d, 1H), 7.11 (s, 1H), 6.82 (d, 1H), 6.75 (s, 1H), 6.59 (s, 2H), 5.19 (d, 1H), 5.10 (d, 1H), 4.48 (m, 1H), 3.96 (m, 1H), 3.80-3.63 (m, 6H), 3.58 (m, 2H), 3.45 (m, 2H), 2.25 (s, 6H). LC-MS: m/z=523.3 (M+H⁺).

### Preparation of Compound 275

### 4-[3-[(2R,3R,4R,5R,6R)-3,5-Dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenyl]-N-methyl-benzamide

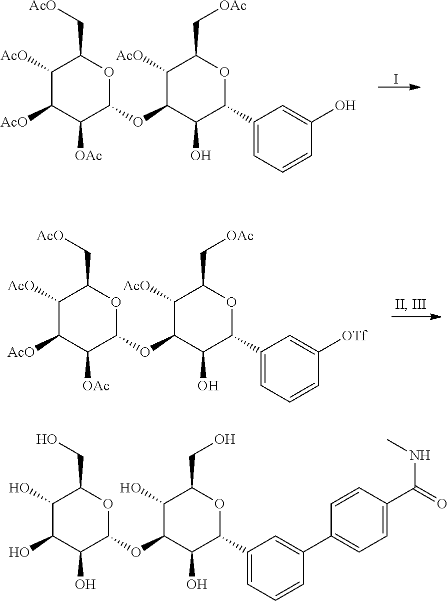

### Step I: (2R,3S,4S,5R,6R)-2-(((2R,3R,4R,5R,6R)-3-acetoxy-2-(acetoxymethyl)-5-hydroxy-6-(3-(((trifluoromethyl)sulfonyl)oxy)phenyl)tetrahydro-2H-pyran-4-yl)oxy)-6-(acetoxymethyl)tetrahydro-2H-pyran-3,4,5-triyltriacetate

To a solution of [(2R,3R,4R,5R,6R)-3-acetoxy-5-hydroxy-6-(3-hydroxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate (COMPOUND 256, Step 1V) (858 mg, 1.279 mmol) in 10 mL of DCM are added 1,1,1-trifluoro-N-phenyl-N-(trifluoromethylsulfonyl)methanesulfonamide (594.1 mg, 1.663 mmol) and TEA (445.7 μL, 3.198 mmol). The mixture is stirred at room temperature overnight. After removal of the solvent under reduced pressure, the residue is separated on Biotage SNAP 100 g silica gel cartridge using a gradient of ethyl acetate/hexane (0-50% in 20 CV) to obtain a inseparable mixture (800 mg) containing two fractions with the desired mass, which is used directly in the next step without further purification.

LC-MS: m/z=825 (M+Na⁺).

### Step II

To a solution of (2R,3S,4S,5R,6R)-2-(((2R,3R,4R,5R,6R)-3-acetoxy-2-(acetoxymethyl)-5-hydroxy-6-(3-(((trifluoromethyl)sulfonyl)oxy)phenyl)tetrahydro-2H-pyran-4-yl)oxy)-6-(acetoxymethyl)tetrahydro-2H-pyran-3,4,5-triyltriacetate (60 mg, 0.075 mmol) in 3 mL of dioxane are added [4-(methylcarbamoyl)phenyl]boronic acid (20.06 mg, 0.1121 mmol), PdCl₂(dppf)₂-DCM (6.10 mg, 0.00748 mmol) and sodium carbonate (112.1 μL of 2 M, 0.2242 mmol). The mixture is stirred at 90° C. overnight under nitrogen. After removal of the solvent under reduced pressure, the residue is purified on Biotage SNAP 10 g silica gel cartridge using a gradient of MeOH/DCM (0-6% in 25 CV) to obtain a mixture (30 mg) containing several compounds with the same mass, which is used directly in the next step without further purification.

LC-MS: m/z=788.4 (M+Na⁺).

### Step III: Compound 275

To a solution of the above-mentioned mixture (30 mg) (from Step II) in methanol (3 mL) is added a drop of 25% sodium methoxide/methanol. After stirring for 20 min at rt, it is neutralized with Amberlite IR120 (H). After filtration, the solvent is removed under reduced pressure and the residue is purified using reverse-phase prep-HPLC to obtain 4-[3-[(2R,3R,4R,5R,6R)-3,5-dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenyl]-N-methyl-benzamide (4.9 mg) as a white solid.

¹H NMR (400 MHz, CD₃OD) δ 7.86 (m, 3H), 7.75 (m, 2H), 7.58 (d, 1H), 7.46 (m, 2H), 5.28 (d, 1H), 5.14 (d, 1H), 4.59 (d, 1H), 3.97 (m, 1H), 3.87 (m, 2H), 3.68 (m, 3H), 3.60 (m, 3H), 3.40 (m, 2H), 2.92 (s, 3H). LC-MS: m/z=536.3 (M+H⁺).

### Preparation of Compound 276

### (2R,3S,4S,5S,6R)-2-[(2R,3R,4R,5R,6R)-3,5-dihydroxy-2-(hydroxymethyl)-6-[3-(4-methylsulfonylphenyl)phenyl]tetrahydropyran-4-yl]oxy-6-(hydroxymethyl)tetrahydropyran-3,4,5-triol

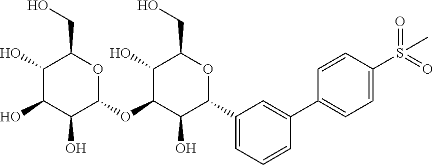 [SMILES: CS(=O)(=O)c1ccc(-c2cccc([C@H]3O[C@H](CO)[C@@H](O)[C@H](O[C@H]4O[C@H](CO)[C@@H](O)[C@H](O)[C@@H]4O)[C@@H]3O)c2)cc1]

The title compound is prepared as described in COMPOUND 275 but using (4-(methylsulfonyl)phenyl)boronic acid as reagent in Step II.

¹H NMR (400 MHz, CD₃OD): δ 7.93 (d, 2H), 7.84 (d, 2H), 7.79 (s, 1H), 7.54 (d, 1H), 7.50-7.35 (m, 2H), 5.19 (d, 1H), 5.05 (d, 1H), 4.52 (s, 1H), 4.49 (d, 1H), 3.88 (m, 1H), 3.76 (d, 2H), 3.65 (d, 2H), 3.51 (m, 5H), 3.06 (s, 3H). LC-MS: m/z=557.3 (M+H⁺).

### Preparation of Compound 277

### 5-[3-[(2R,3R,4R,5R,6R)-3,5-Dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenyl]-N1,N3-dimethyl-benzene-1,3-dicarboxamide

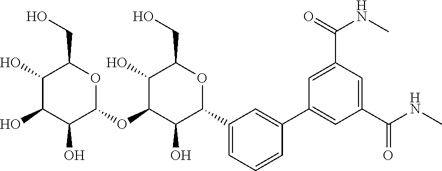 [SMILES: CNC(=O)c1cc(C(=O)NC)cc(-c2cccc([C@H]3O[C@H](CO)[C@@H](O)[C@H](O[C@H]4O[C@H](CO)[C@@H](O)[C@H](O)[C@@H]4O)[C@@H]3O)c2)c1]

The title compound is prepared as described in COMPOUND 275 but using (3,5-bis(methylcarbamoyl)phenyl)boronic acid as reagent in Step II.

¹H NMR (400 MHz, CD₃OD): δ 8.23 (m, 3H), 7.87 (s, 1H), 7.64 (d, 1H), 7.57-7.42 (m, 2H), 5.24 (d, 1H), 5.14 (s, 1H), 4.61 (s, 1H), 4.56 (d, 1H), 3.96 (m, 1H), 3.86 (m, 2H), 3.76 (m, 2H), 3.69-3.59 (m, 3H), 3.54 (m, 2H), 2.95 (s, 6H). LC-MS: m/z=593.3 (M+H⁺).

### Preparation of Compound 278

### (2R,3S,4S,5S,6R)-2-[(2R,3R,4R,5R,6R)-2-(3-Cyclohexylphenyl)-3,5-dihydroxy-6-(hydroxymethyl)tetrahydropyran-4-yl]oxy-6-(hydroxymethyl)tetrahydropyran-3,4,5-triol

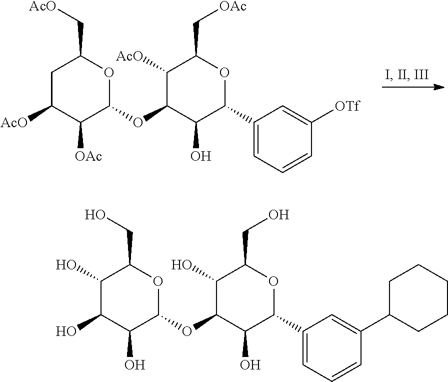

### Step I

To a solution of [(2R,3R,4R,5R,6R)-3-acetoxy-5-hydroxy-4-[(2R,3S,4S,5R,6R)-3,4,5-triacetoxy-6-(acetoxymethyl)tetrahydropyran-2-yl]oxy-6-[3-(trifluoromethylsulfonyloxy)phenyl]tetrahydropyran-2-yl]methyl acetate (80 mg, 0.09967 mmol) in 3 mL of dioxane are added 1-cyclohexenylboronic acid (18.83 mg, 0.1495 mmol), PdCl₂(dppf)₂-DCM (8.14 mg, 0.009967 mmol) and sodium carbonate (149.5 μL of 2 M, 0.2990 mmol). The mixture is stirred at 90° C. overnight under nitrogen. After removal of the solvent under reduced pressure, the residue is purified on Biotage SNAP 25 g silica gel cartridge using a gradient of MeOH/DCM (0-6% in 25 CV) to obtain a mixture (62 mg) containing the right mass, which is used directly in the next step without further purification.

### Step II

To a solution of the above-mentioned mixture (62 mg) in methanol is added a catalytic amount of 10% Pd/C. The flask is attached to a hydrogen balloon for hydrogenation. The mixture is stirred at rt for 1 h. After filtration, the filtrate is concentrated to dryness and the residue is purified on Biotage SNAP 25 g silica gel cartridge using a gradient of MeOH/DCM (0-6% in 25 CV) to obtain a mixture (41 mg), which is used directly in the next step without further purification.

### Step III: Compound 278

To a solution of the above-mentioned mixture (41 mg) in methanol (3 mL) is added a catalytic amount of 25% sodium methoxide/methanol. After stirring for 30 min, it is neutralized with resin Amberlite IR120 (H). After filtration, the solvent is removed under reduced pressure and the residue is purified using reverse-phase prep-HPLC to obtain (2R,3S,4S,5S,6R)-2-[(2R,3R,4R,5R,6R)-2-(3-cyclohexylphenyl)-3,5-dihydroxy-6-(hydroxymethyl)tetrahydropyran-4-yl]oxy-6-(hydroxymethyl)tetrahydropyran-3,4,5-triol (2 mg) as a white solid.

¹H NMR (400 MHz, CD₃OD): δ 7.26 (s, 1H), 7.16 (m, 2H), 7.02 (d, 1H), 5.12 (d, 1H), 5.05 (d, 1H), 4.47 (m, 1H), 3.90 (m, 1H), 3.79-3.54 (m, 6H), 3.53-3.45 (m, 2H), 3.39 (m, 2H), 2.42 (m, 1H), 1.70 (m, 5H), 1.41-1.14 (m, 5H). LC-MS: m/z=485.4 (M+H⁺).

### Preparation of Compound 279

### (2R,3S,4S,5S,6R)-2-[(2R,3R,4R,5R,6R)-3,5-Dihydroxy-2-(hydroxymethyl)-6-(3-pyrimidin-5-ylphenyl)tetrahydropyran-4-yl]oxy-6-(hydroxymethyl)tetrahydropyran-3,4,5-triol

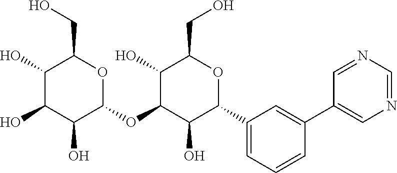 [SMILES: OC[C@H]1O[C@H](O[C@@H]2[C@H](O)[C@@H](c3cccc(-c4cncnc4)c3)O[C@H](CO)[C@H]2O)[C@@H](O)[C@@H](O)[C@@H]1O]

The title compound is prepared as described in COMPOUND 275 but using pyrimidin-5-ylboronic acid as reagent in Step II.

¹H NMR (400 MHz, CD₃OD) δ 9.03 (m, 3H), 7.79 (s, 1H), 7.59-7.40 (m, 3H), 5.17 (d, 1H), 5.03 (d, 1H), 4.46 (d, 1H), 3.87 (m, 1H), 3.81-3.73 (m, 2H), 3.70-3.62 (m, 2H), 3.57-3.33 (m, 6H). LC-MS: m/z=481.3 (M+H⁺).

### Preparation of Compound 280

### (2R,3S,4S,5S,6R)-2-[(2R,3R,4R,5R,6R)-3,5-Dihydroxy-2-(hydroxymethyl)-6-[3-(3-methylbenzimidazol-5-yl)phenyl]tetrahydropyran-4-yl]oxy-6-(hydroxymethyl)tetrahydropyran-3,4,5-triol (Trifluoroacetate Ion (1))

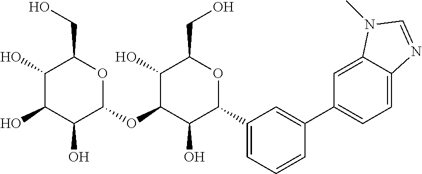 [SMILES: Cn1cnc2ccc(-c3cccc([C@H]4O[C@H](CO)[C@@H](O)[C@H](O[C@H]5O[C@H](CO)[C@@H](O)[C@H](O)[C@@H]5O)[C@@H]4O)c3)cc21]

The title compound is prepared as described in COMPOUND 275 but using (1-methyl-1H-benzo[d]imidazol-6-yl)boronic acid as reagent in Step II.

1H NMR (400 MHz, CD₃OD) δ 9.01 (s, 1H), 8.02 (s, 1H), 7.79 (m, 3H), 7.58 (d, 1H), 7.46-7.36 (m, 2H), 5.19 (d, 1H), 5.05 (d, 1H), 4.49 (d, 1H), 4.05 (s, 3H), 3.89 (m, 1H), 3.81-3.76 (m, 2H), 3.71-3.63 (m, 3H), 3.60-3.50 (m, 3H), 3.45 (m, 2H). LC-MS: m/z=533.3 (M+H⁺).

### Preparation of Compound 281

### (2R,3S,4S,5S,6R)-2-[(2R,3R,4R,5R,6R)-2-Allyl-3,5-dihydroxy-6-(hydroxymethyl)tetrahydropyran-4-yl]oxy-6-(hydro I am ymethyl)tetrahydropyran-3,4,5-triol

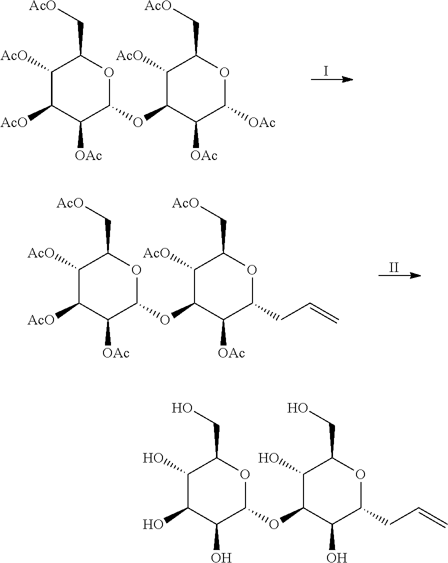

### Step I: [(2R,3R,4R,5R,6R)-3,5-Diacetoxy-6-allyl-4-[(2R,3S,4S,5R,6R)-3,4,5-triacetoxy-6-(acetoxymethyl)tetrahydropyran-2-yl]oxy-tetrahydropyran-2-yl]methyl acetate

To a stirred solution of [(2R,3R,4S,5S,6R)-3,4,5-triacetoxy-6-[(2R,3S,4S,5R,6R)-2,3,5-triacetoxy-6-(acetoxymethyl)tetrahydropyran-4-yl]oxy-tetrahydropyran-2-yl]methyl acetate (500 mg, 0.7368 mmol) and allyl-trimethyl-silane (351.2 μL, 2.210 mmol) in 10 mL of dry acetonitrile is added TMS triflate (159.8 μL, 0.8842 mmol) at 0° C. The mixture is stirred at rt overnight and quenched with TEA (102.7 μL, 0.7368 mmol). After removal of the solvent under reduced pressure, the residue is purified on Biotage SNAP 25 g silica gel cartridge using a gradient of ethyl acetate/hexane (0-50% in 20 CV) to afford the title compound.

LC-MS: m/z=683.3 (M+Na⁺).

### Step II: Compound 281

To a stirred solution of the compound (31 mg) (from Step I) in methanol (3 mL) is added a drop of 25% sodium methoxide. The mixture is stirred at rt overnight. It is then neutralized with resin Amberlite IR120 (H) and filtered. The filtrate is concentrated to dryness under reduced pressure. The residue is purified using reverse-phase HPLC to afford the title compound (3 mg) as a white solid.

1H NMR (CD₃OD, 400 MHz): 5.79 (m, 1H), 4.96-5.07 (m, 3H), 3.85 (m, 2H), 3.56-3.80 (m, 8H), 3.45 (m, 3H), 2.42 (m, 2H). LC-MS: m/z=367.2 (M+H⁺).

### Preparation of Compound 282

### (2R,3S,4S,5S,6R)-2-[(2R,3R,4R,5R,6R)-2-[(E)-3-Cyclohexylallyl]-3,5-dihydroxy-6-(hydroxymethyl)tetrahydropyran-4-yl]oxy-6-(hydroxymethyl)tetrahydropyran-3,4,5-triol

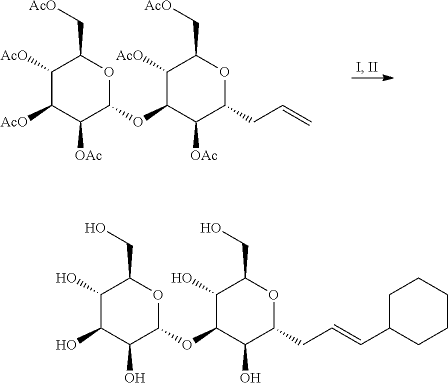

### Step I: [(2R,3R,4R,5R,6R)-3,5-Diacetoxy-6-[(E)-3-cyclohexylallyl]-4-[(2R,3S,4S,5R,6R)-3,4,5-triacetoxy-6-(acetoxymethyl)tetrahydropyran-2-yl]oxy-tetrahydropyran-2-yl]methyl acetate

To a stirred solution of [(2R,3R,4R,5R,6R)-3,5-diacetoxy-6-allyl-4-[(2R,3S,4S,5R,6R)-3,4,5-triacetoxy-6-(acetoxymethyl)tetrahydropyran-2-yl]oxy-tetrahydropyran-2-yl]methyl acetate (Compound 281, Step 1) (40 mg, 0.06055 mmol) in DCM (2 mL) are added vinylcyclohexane (26.69 mg, 0.2422 mmol) and Grubbs catalyst II (5.8 mg, 0.0060 mmol). The mixture is stirred at 40° C. under nitrogen overnight. After removal of the solvent under reduced pressure, the residue is purified on Biotage SNAP 10 g silica gel cartridge using a gradient of MeOH/DCM (0-5% in 20 CV) to afford the title compound (30 mg, 66.7%).

1H NMR (CDCl₃, 400 MHz): 5.47 (m, 1H), 5.15-5.31 (m, 5H), 4.96-5.02 (m, 2H), 4.26 (m, 2H), 3.98-4.07 (m, 5H), 3.78 (m, 1H), 2.38 (m, 1H), 1.90-2.27 (m, 21H), 1.88 (m, 1H), 1.65 (m, 6H), 1.02-1.22 (m, 5H).

### Step II: COMPOUND 282

To a solution of the compound (30 mg) (from Step I) in MeOH (3 mL) is added a drop of 25% NaOMe/MeOH and stirred at rt for 30 min. It is neutralized with resin Amberlite IR-120 (H). After filtration, it is concentrated to dryness under vacuum and the residue is purified using reverse-phase prep-HPLC to afford a 6/1 mixture of the title compound (major) cis analog (minor) (3 mg) as a white solid.

1H NMR (CD₃OD, 400 MHz): 5.43 (m, 1H), 5.33 (m, 1H), 5.02 (s, 1H), 3.85 (m, 2H), 3.48-3.77 (m, 10H), 3.37 (m, 1H), 2.32 (m, 1H), 2.22 (m, 1H), 1.84 (m, 1H), 1.60 (m, 5H), 0.96-1.18 (m, 5H). LC-MS: m/z=449.3 (M+H⁺).

### Preparation of Compound 283

### Methyl 4-[3-[(2R,3R,4R,5R,6R)-3,5-dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]propyl]benzoate

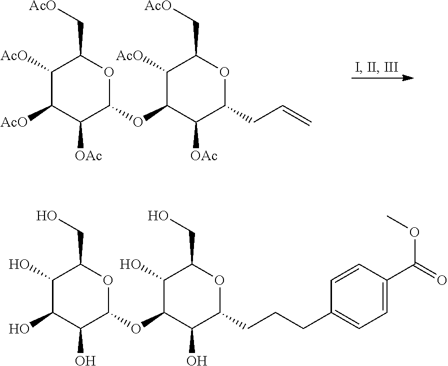

### Step I: Methyl 4-[(E)-3-[(2R,3R,4R,5R,6R)-3,5-diacetoxy-6-(acetoxymethyl)-4-[(2R,3S,4S,5R,6R)-3,4,5-triacetoxy-6-(acetoxymethyl)tetrahydropyran-2-yl]oxy-tetrahydropyran-2-yl]prop-1-enyl]benzoate

To a stirred solution of [(2R,3R,4R,5R,6R)-3,5-diacetoxy-6-allyl-4-[(2R,3S,4S,5R,6R)-3,4,5-triacetoxy-6-(acetoxymethyl)tetrahydropyran-2-yl]oxy-tetrahydropyran-2-yl]methyl acetate (Compound 281, Step 1) (50 mg, 0.07569 mmol) in DCM (5 mL) are added methyl 4-vinylbenzoate (49.11 mg, 0.3028 mmol) and Grubbs catalyst II (6.2 mg, 0.0075 mmol). The mixture is stirred at 40° C. under nitrogen overnight. After removal of the solvent under reduced pressure, the residue is purified on Biotage SNAP 10 g silica gel cartridge using a gradient of MeOH/DCM (0-5% in 20 CV) to afford the title compound (50 mg, 83.1%).

LC-MS: m/z=817.3 (M+Na⁺).

### Step II: Methyl 4-[3-[(2R,3R,4R,5R,6R)-3,5-diacetoxy-6-(acetoxymethyl)-4-[(2R,3S,4S,5R,6R)-3,4,5-triacetoxy-6-(acetoxymethyl)tetrahydropyran-2-yl]oxy-tetrahydropyran-2-yl]propyl]benzoate

To a solution of the product (50 mg) (from Step I) in MeOH (3 mL) is added a catalytic amount of 10% Pd/C. Then the mixture is hydrogenated using a hydrogen balloon. After stirring at rt for 30 min, the mixture is filtered, and the solvent is removed under reduced pressure. The residue is purified on Biotage SNAP 10 g silica gel cartridge using a gradient of MeOH/DCM (0-5% in 20 CV) to obtain afford the title compound (45 mg), which is used in the next step without further purification.

LC-MS: m/z=819.4 (M+Na⁺).

### Step III: Compound 283

To a stirred solution of methyl 4-[3-[(2R,3R,4R,5R,6R)-3,5-diacetoxy-6-(acetoxymethyl)-4-[(2R,3S,4S,5R,6R)-3,4,5-triacetoxy-6-(acetoxymethyl)tetrahydropyran-2-yl]oxy-tetrahydropyran-2-yl]propyl]benzoate (45 mg, 0.07530 mmol) in 2 mL of MeOH is added one drop of 25% sodium methoxide/methanol. The mixture is stirred at rt for 20 min. It is then neutralized with Amberlite IR120 (H). After filtration, the solvent is removed under reduced pressure and the residue is purified using reverse-phase prep-HPLC to afford the title compound (6.8 mg) as a white solid.

¹H NMR (400 MHz, CD₃OD): δ 7.91 (d, 2H), 7.32 (d, 2H), 5.10-5.03 (m, 1H), 3.97-3.89 (m, 2H), 3.86 (s, 3H), 3.78 (m, 7H), 3.65 (m, 6.2 Hz, 2H), 3.55 (m, 1H), 3.37 (m, 1H), 2.73 (m, 2H), 1.91-1.60 (m, 3H), 1.57-1.41 (m, 1H). LC-MS: m/z=503.3 (M+H⁺).

### Preparation of Compound 284

### (2R,3S,4S,5S,6R)-2-[(2R,3R,4R,5R,6R)-3,5-Dihydroxy-2-(hydroxymethyl)-6-(3-methoxyphenyl)tetrahydropyran-4-yl]oxy-6-(hydroxymethyl)tetrahydropyran-3,4,5-triol

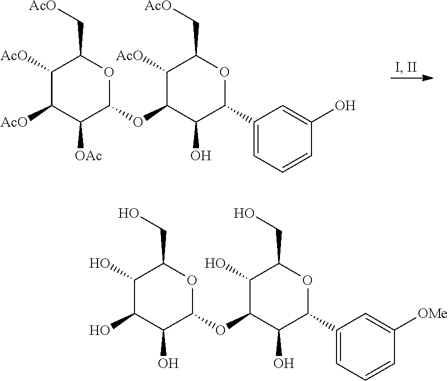

### Step I: [(2R,3R,4R,5R,6R)-3-acetoxy-5-hydroxy-6-(3-methoxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate

To a solution of [(2R,3R,4R,5R,6R)-3-acetoxy-5-hydroxy-6-(3-hydroxyphenyl)-4-I [(2R,3S,4S,5R,6R)-3,4,5-triacetoxy-6-(acetoxymethyl)tetrahydropyran-2-yl]oxy-tetrahydropyran-2-yl]methyl acetate (COMPOUND 265, Step 1V) (58 mg, 0.086 mmol) in DMF (1.16 mL) is added Cs₂CO₃ (42.2 mg, 0.13 mmol) followed by MeI (8.1 μL, 0.130 mmol). The reaction mixture is stirred at 50° C. overnight, and then diluted with EtOAC, washed with H₂O and brine consecutively. The organic phase is dried over Na₂SO₄, filtered and concentrated. The residue is purified on Biotage SNAP 10 g silica gel cartridge using MeOH in CH₂Cl₂ (0 to 8% in 20 CV) to afford the expected compound as a mixture of compounds having the same mass, which is used in the next step without any further purification.

LC-MS: m/z=685.3 (M+H⁺).

### Step II: Compound 284

To a solution of [(2R,3R,4R,5R,6R)-3-acetoxy-5-hydroxy-6-(3-methoxyphenyl)-4-[(2R,3S,4S,5R,6R)-3,4,5-triacetoxy-6-(acetoxymethyl)tetrahydropyran-2-yl]oxy-tetrahydropyran-2-yl]methyl acetate (47 mg, 0.068 mmol) in MeOH (940 μL) is added dry K₂CO₃ (9.4 mg, 0.068 mmol). The reaction mixture is stirred at rt for 1 h, then neutralized with resin Amberlite IR-120 (H), filtered and dried. The residue is purified by preparative HPLC to afford 2R,3S,4S,5S,6R)-2-[(2R,3R,4R,5R,6R)-3,5-dihydroxy-2-(hydroxymethyl)-6-(3-methoxyphenyl)tetrahydropyran-4-yl]oxy-6-(hydroxymethyl)tetrahydropyran-3,4,5-triol (29.6 mg, 54% yield).

1H NMR (400 MHz, CD₃OD): δ 7.25 (t, 1H), 7.12-6.96 (m, 2H), 6.81 (m, 1H), 5.19 (d, 1H), 5.12 (d, 1H), 4.52 (t, 1H), 3.97 (m, 1H), 3.85-3.79 (m, 1H), 3.78 (s, 3H), 3.75-3.36 (m, 9H).

LC-MS: m/z=433.2 (M+H⁺).

### Preparation of Compound 285 to 288

The title compounds are prepared as described in COMPOUND 284 but using the appropriate commercially available alkylating reagents.

### Compound 285

### (2R,3S,4S,5S,6R)-2-[(2R,3R,4R,5R,6R)-2-[3-(Cyclopentylmethoxy)phenyl]-3,5-dihydroxy-6-(hydroxymethyl)tetrahydropyran-4-yl]oxy-6-(hydroxymethyl)-tetrahydropyran-3,4,5-triol

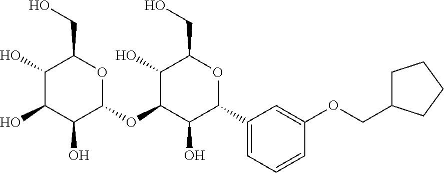 [SMILES: OC[C@H]1O[C@H](O[C@@H]2[C@H](O)[C@@H](c3cccc(OCC4CCCC4)c3)O[C@H](CO)[C@H]2O)[C@@H](O)[C@@H](O)[C@@H]1O]

¹H NMR (400 MHz, CD₃OD): δ 7.15 (t, 1H), 6.99 (s, 1H), 6.91 (d, 1H), 6.71 (d, 1H), 5.10 (d, 1H), 5.04 (d, 1H), 4.48-4.40 (m, 1H), 3.89 (m, 1H), 3.83-3.29 (m, 12H), 2.35-2.12 (m, 2H), 1.75 (d, 2H), 1.66-1.42 (m, 3H), 1.30 (m, 2H). LC-MS: m/z=501.4 (M+H⁺)

### Compound 286

### Methyl 2-[3-[(2R,3R,4R,5R,6R)-3,5-dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenoxy]acetate

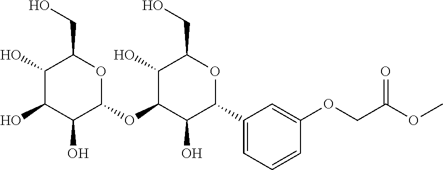 [SMILES: COC(=O)COc1cccc([C@H]2O[C@H](CO)[C@@H](O)[C@H](O[C@H]3O[C@H](CO)[C@@H](O)[C@H](O)[C@@H]3O)[C@@H]2O)c1]

¹H NMR (400 MHz, CD₃OD): δ 7.27 (m, 1H), 7.07 (d, 2H), 6.91-6.69 (m, 1H), 5.15 (m, 2H), 4.71 (s, 2H), 4.51 (t, 1H), 3.96 (m, 1H), 3.90-3.79 (m, 1H), 3.8 (s, 3H), 3.74-3.35 (m, 9H). LC-MS: m/z=491.3 (M+H⁺)

### Compound 287

### (2R,3S,4S,5S,6R)-2-[(2R,3R,4R,5R,6R)-2-[3-(2-Fluoroethoxy)phenyl]-3,5-dihydroxy-6-(hydroxymethyl)tetrahydropyran-4-yl]oxy-6-(hydroxymethyl)tetrahydropyran-3,4,5-triol

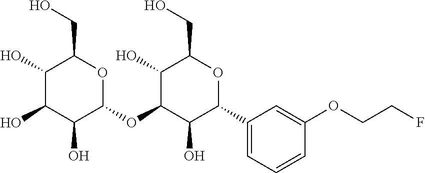 [SMILES: OC[C@H]1O[C@H](O[C@@H]2[C@H](O)[C@@H](c3cccc(OCCF)c3)O[C@H](CO)[C@H]2O)[C@@H](O)[C@@H](O)[C@@H]1O]

¹H NMR (400 MHz, CD₃OD): δ 7.19 (m, 1H), 7.06-6.92 (m, 2H), 6.76 (m, 1H), 5.11 (d, 1H), 5.03 (m, 1H), 4.72-4.63 (m, 1H), 4.62-4.51 (m, 1H), 4.48-4.36 (m, 1H), 4.21-4.13 (m, 1H), 4.13-4.07 (m, 1H), 3.89 (m, 1H), 3.80-3.29 (m, 10H). LC-MS: m/z=465.3 (M+H⁺)

### Compound 288

### 2-[3-[(2R,3R,4R,5R,6R)-3,5-dihydroxy-6-(hydroxymethyl)-4-[(2R,3S,4S,5S,6R)-3,4,5-trihydroxy-6-(hydroxymethyl)tetrahydropyran-2-yl]oxy-tetrahydropyran-2-yl]phenoxy]-N-methyl-acetamide

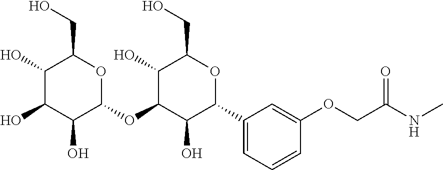 [SMILES: CNC(=O)COc1cccc([C@H]2O[C@H](CO)[C@@H](O)[C@H](O[C@H]3O[C@H](CO)[C@@H](O)[C@H](O)[C@@H]3O)[C@@H]2O)c1]

¹H NMR (400 MHz, CD₃OD) δ 8.06 (s, 1H), 7.21 (t, 1H), 7.10-6.96 (m, 2H), 6.80 (m, 1H), 5.05 (m, 2H), 4.45-4.29 (m, 3H), 3.88 (m, 1H), 3.74 (m, 2H), 3.69-3.53 (m, 5H), 3.53-3.32 (m, 3H), 2.72 (d, 3H). LC-MS: m/z=490.3 (M+H⁺)

### Preparation of Compound 289

### (2R,3S,4S,5S,6R)-2-[(2R,3R,4R,5R,6R)-3,5-Dihydroxy-2-(3-hydroxy-4-methoxy-phenyl)-6-(hydroxymethyl)tetrahydropyran-4-yl]oxy-6-(hydroxymethyl)tetrahydropyran-3,4,5-triol

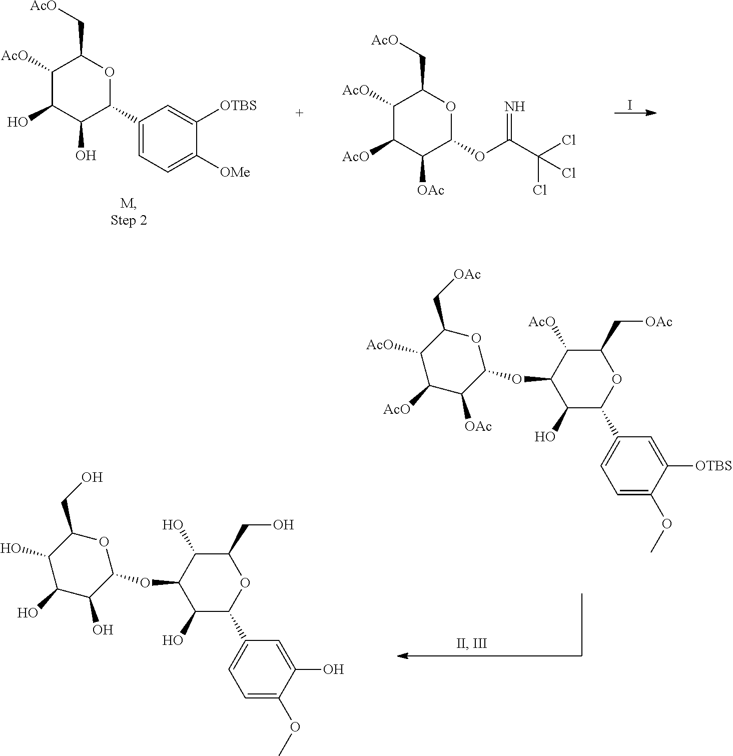

### Step I: [(2R,3R,4R,5R,6R)-3-acetoxy-6-[3-[tert-butyl(dimethyl)silyl]oxy-4-methoxy-phenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate

To a stirred solution of [(2R,3R,4S,5S,6R)-3,4,5-triacetoxy-6-(2,2,2-trichloroethanimidoyl)oxy-tetrahydropyran-2-yl]methyl acetate (INTERMEDIATE M, Step II) (500.2 mg, 0.8629 mmol) and [(2R,3R,4S,5S,6R)-3,4,5-triacetoxy-6-(2,2,2-trichloroethanimidoyl)oxy-tetrahydropyran-2-yl]methyl acetate (500.2 mg, 0.8629 mmol) in DCM (18.04 mL) is added 4 molecular sieves powder (1 g), stirred at rt for 30 min. After cooling to −40° C., trimethylsilyl trifluoromethanesulfonate (15.6 μL, 0.08629 mmol) is added dropwise to the mixture. It is stirred at −40° C. and slowly warmed up to −5° C. in 1 h. Then NEt₃ (120.3 μL, 0.8629 mmol) is added, and the mixture is warmed up to rt, filtered off to remove the molecular sieves, and concentrated to dryness. The residue is purified on Biotage SNAP 50 g silica gel cartridge using MeOH/DCM (0 to 7% in 20 CV) to provide an inseparable mixture containing the title compound, which is used in the next step without further purification.

LC-MS: m/z=815.5 (M+H⁺).

### Step II: [(2R,3R,4R,5R,6R)-3-acetoxy-5-hydroxy-6-(3-hydroxy-4-methoxy-phenyl)-4-[(2R,3S,4S,5R,6R)-3,4,5-triacetoxy-6-(acetoxymethyl)tetrahydropyran-2-yl]oxy-tetrahydropyran-2-yl]methyl acetate

To a solution of [(2R,3R,4R,5R,6R)-3-acetoxy-6-[3-[tert-butyl(dimethyl)silyl]oxy-4-methoxy-phenyl]-5-hydroxy-4-[(2R,3S,4S,5R,6R)-3,4,5-triacetoxy-6-(acetoxymethyl)tetrahydropyran-2-yl]oxy-tetrahydropyran-2-yl]methyl acetate (0.75 g, 0.9204 mmol) in THF (22.50 mL) is added 1M TBAF/THF (1.841 mL of 1 M, 1.841 mmol) and AcOH (52.3 μL, 0.92 mmol). The reaction mixture is stirred at rt overnight. After removal of the solvent under reduced pressure, the residue is dissolved in DCM, washed with H₂O and brine consecutively, dried over Na₂SO₄, filtered and dried. The residue is purified on Biotage SNAP 25 g silica gel cartridge using MeOH in CH₂Cl₂ (0-10% in 25 CV) to afford the title compound as an inseparable mixture.

LC-MS: m/z=701.4 (M+H⁺).

### Step V: Compound 289

To a solution of [(2R,3R,4R,5R,6R)-3-acetoxy-5-hydroxy-6-(3-hydroxy-4-methoxy-phenyl)-4-[(2R,3S,4S,5R,6R)-3,4,5-triacetoxy-6-(acetoxymethyl)tetrahydropyran-2-yl]oxy-tetrahydropyran-2-yl]methyl acetate (36 mg, 0.05138 mmol) in MeOH (720.0 μL) is added dry K₂CO₃ (7.1 mg, 0.051 mmol). The reaction mixture is stirred at rt for 1 h, then neutralized with resin Amberlite 120 (H), filtered and dried. The residue is purified by preparative HPLC to afford the title compound (12 mg, 52% yield).

¹H NMR (400 MHz, CD₃OD): δ 6.99-6.79 (m, 3H), 5.10 (d, 2H), 4.46 (s, 1H), 3.96 (m, 1H), 3.87-3.73 (m, 5H), 3.75-3.62 (m, 4H), 3.59 (m, 2H), 3.53-3.44 (m, 1H), 3.43-3.34 (m, 1H). LC-MS: m/z=449.3 (M+H⁺).

### Preparation of Compound 290

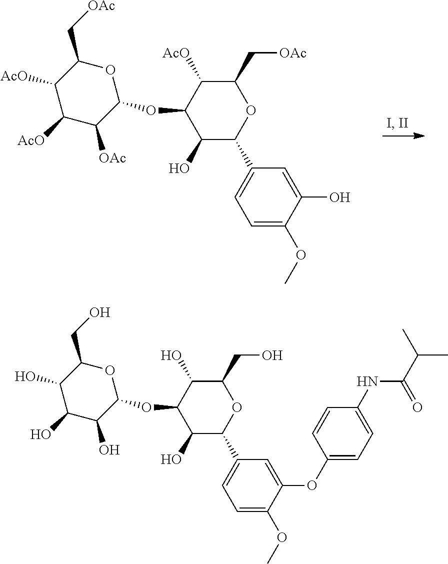 [SMILES: COc1ccc([C@H]2O[C@H](COC(C)=O)[C@@H](OC(C)=O)[C@H](O[C@H]3O[C@H](COC(C)=O)[C@@H](OC(C)=O)[C@H](OC(C)=O)[C@@H]3OC(C)=O)[C@@H]2O)cc1O ; COc1ccc([C@H]2O[C@H](CO)[C@@H](O)[C@H](O[C@H]3O[C@H](CO)[C@@H](O)[C@H](O)[C@@H]3O)[C@@H]2O)cc1Oc1ccc(NC(=O)C(C)C)cc1]

### Step I: [(2R,3R,4R,5R,6R)-3-acetoxy-5-hydroxy-6-[4-methoxy-3-[4-(2-methylpropanoylamino)phenoxy]phenyl]-4-[(2R,3S,4S,5R,6R)-3,4,5-triacetoxy-6-(acetoxymethyl)tetrahydropyran-2-yl]oxy-tetrahydropyran-2-yl]methyl acetate

To a solution of [(2R,3R,4R,5R,6R)-3-acetoxy-5-hydroxy-6-(3-hydroxy-4-methoxy-phenyl)-4-[(2R,3S,4S,5R,6R)-3,4,5-triacetoxy-6-(acetoxymethyl)tetrahydropyran-2-yl]oxy-tetrahydropyran-2-yl]methyl acetate (COMPOUND 289, Step II) (70 mg, 0.10 mmol) and [4-(2-methylpropanoylamino)phenyl]boronic acid (41.37 mg, 0.20 mmol) in DCM (5 mL) are added 4 molecular sieves powder (400 mg) and Cu(OAc)₂ (25.41 mg, 0.1399 mmol). After stirring for 10 min, 2,6-lutidine (53.53 mg, 57.9 μL, 0.50 mmol) is added to the mixture and stirred at rt for 20 h. The reaction mixture is filtered off to remove the molecular sieves, and concentrated to dryness. The residue is purified on Biotage SNAP 10 g silica gel cartridge using MeOH/DCM (0 to 7% in 20 CV) to provide an inseparable mixture containing expected compound (63.5 mg).

### Step II: Compound 290

To a solution of [(2R,3R,4R,5R,6R)-3-acetoxy-5-hydroxy-6-[4-methoxy-3-[4-(2-methylpropanoylamino)phenoxy]phenyl]-4-[(2R,3S,4S,5R,6R)-3,4,5-triacetoxy-6-(acetoxymethyl)tetrahydropyran-2-yl]oxy-tetrahydropyran-2-yl]methyl acetate (63.5 mg, 0.07368 mmol) in MeOH (1.270 mL) is added dry K₂CO₃ (10.18 mg, 0.07368 mmol). The mixture is stirred at rt for 1 h, neutralized with resin Amberlite 120 (H), filtered and dried. The residue is purified by preparative HPLC to afford the title compound (20.4 mg, 45.0%) as a white powder after lyophylization.

1H NMR (400 MHz, CD₃OD) δ 9.64 (s, 1H), 7.43 (m, 2H), 7.24 (d, 1H), 7.10 (m, 2H), 6.88-6.71 (m, 2H), 5.09 (m, 2H), 4.42 (s, 1H), 3.94 (m, 1H), 3.83-3.32 (m, 13H), 2.58 (m, 1H), 1.17 (d, 6H). LC-MS: m/z=610.4 (M+H⁺).

### Preparation of Compound 291

### 5-(3-((2R,3S,4R,5S,6R)-3,4,5-trihydroxy-6-(hydroxymethyl)tetrahydro-2H-pyran-2-yl)phenoxy)isophthalic acid

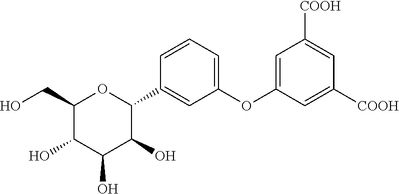 [SMILES: O=C(O)c1cc(Oc2cccc([C@H]3O[C@H](CO)[C@@H](O)[C@H](O)[C@@H]3O)c2)cc(C(=O)O)c1]

Dimethyl 5-[3-[(2R,3S,4R,5S,6R)-3,4,5-trihydroxy-6-(hydroxymethyl)tetrahydropyran-2-yl]phenoxy]benzene-1,3-dicarboxylate (Compound 51) (26.9 mg, 0.05159 mmol) is dissolved in THF (463 μL) and H₂O (463 μL). LiOH (10.8 mg, 0.2580 mmol) is added and the mixture is stirred overnight. The resulting mixture is acidified with 65 μL of HCl (4M) and then purified by reverse phase HPLC to afford the title compound (15.6 mg, 66%)

LC-MS: m/z=421.25 (M+H⁺).

### Preparation of Compound 292

### N-methyl-3-(4-((2R,3S,4R,5S,6R)-3,4,5-trihydroxy-6-(hydroxymethyl)tetrahydro-2H-pyran-2-yl)phenoxy)benzamide

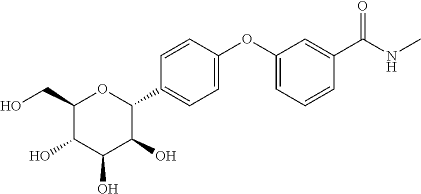 [SMILES: CNC(=O)c1cccc(Oc2ccc([C@H]3O[C@H](CO)[C@@H](O)[C@H](O)[C@@H]3O)cc2)c1]

The title compound is prepared using similar procedure as described for COMPOUND 3 but using ((2R,3S,4R,5S,6R)-3-acetoxy-4,5-dihydroxy-6-(4-hydroxyphenyl)tetrahydro-2H-pyran-2-yl)methyl acetate (INTERMEDIATE L, Step II) and (3-(methylcarbamoyl)phenyl)boronic acid as the appropriate starting materials.

¹H NMR (400 MHz, DMSO-D₆) δ 8.45 (m, 1H), 7.56 (m, 1H), 7.43 (m, 4H), 7.13 (m, 1H), 6.99 (m, 2H), 4.66 (d, J=5.6 Hz, 1H), 4.00 (dd, J=5.6, 3.0 Hz, 1H), 3.69-3.50 (m, 3H), 3.50-3.35 (m, 2H), 2.73 (m, 3H). LC-MS: m/z=390.25 (M+H⁺).

### Preparation of Compound 293

### methyl 2-(3-(4-((2R,3S,4R,5S,6R)-3,4,5-trihydroxy-6-(hydroxymethyl)tetrahydro-2H-pyran-2-yl)phenoxy)phenyl)acetate

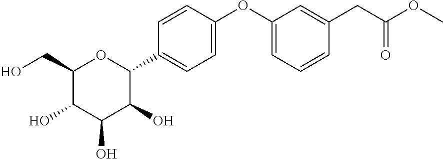 [SMILES: COC(=O)Cc1cccc(Oc2ccc([C@H]3O[C@H](CO)[C@@H](O)[C@H](O)[C@@H]3O)cc2)c1]

The title compound is prepared using similar procedure as described for COMPOUND 3 but using ((2R,3S,4R,5S,6R)-3-acetoxy-4,5-dihydroxy-6-(4-hydroxyphenyl)tetrahydro-2H-pyran-2-yl)methyl acetate (INTERMEDIATE L, Step II) and (3-(2-methoxy-2-oxoethyl)phenyl)boronic acid as the appropriate starting materials.

¹H NMR (400 MHz, DMSO-D₆) δ 7.38 (m, 2H), 7.31 (m, 1H), 6.95 (m, 4H), 6.86 (m, 1H), 4.64 (d, J=5.4 Hz, 1H), 3.99 (m, 1H), 3.66 (s, 2H), 3.64-3.51 (m, 6H), 3.47-3.37 (m, 2H). LC-MS: m/z=405.31 (M+H⁺).

### Preparation of Compound 294

### methyl 4′-((2R,3S,4R,5S,6R)-3,4,5-trihydroxy-6-(hydroxymethyl)tetrahydro-2H-pyran-2-yl)-[1,1′-biphenyl]-3-carboxylate

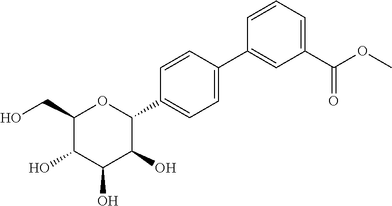 [SMILES: COC(=O)c1cccc(-c2ccc([C@H]3O[C@H](CO)[C@@H](O)[C@H](O)[C@@H]3O)cc2)c1]

The title compound is prepared following the procedure described in COMPOUND 158 using INTERMEDIATE 0 and methyl 3-bromobenzoate as the appropriate starting materials.

¹H NMR (400 MHz, DMSO-D₆) δ 8.13 (t, J=1.6 Hz, 1H), 7.90 (m, 2H), 7.63 (d, J=8.4 Hz, 2H), 7.57 (t, J=7.8 Hz, 1H), 7.48 (d, J=8.2 Hz, 2H), 4.69 (m, 1H), 4.03 (m, 1H), 3.83 (s, 3H), 3.58 (m, 2H), 3.56-3.47 (m, 2H), 3.43-3.34 (m, 1H). LC-MS: m/z=375.3 (M+H⁺).

### Preparation of Compound 295

### N-methyl-4′-((2R,3S,4R,5S,6R)-3,4,5-trihydroxy-6-(hydroxymethyl)tetrahydro-2H-pyran-2-yl)-[1,1′-biphenyl]-3-carboxamide

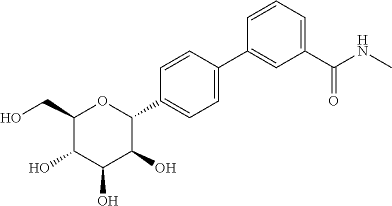 [SMILES: CNC(=O)c1cccc(-c2ccc([C@H]3O[C@H](CO)[C@@H](O)[C@H](O)[C@@H]3O)cc2)c1]

The title compound is prepared following the procedure described in COMPOUND 158 using INTERMEDIATE 0 and 3-bromo-N-methylbenzamide as the appropriate starting materials.

¹H NMR (400 MHz, DMSO-D₆) δ 8.53 (d, J=4.4 Hz, 1H), 8.08 (s, 1H), 7.77 (m, 2H), 7.68 (d, J=8.3 Hz, 2H), 7.49 (m, 3H), 4.74 (m, 3H), 4.62 (m, 2H), 4.08 (s, 1H), 3.55 (m, 3H), 3.40 (m, 2H), 2.77 (d, J=4.5 Hz, 3H). LC-MS: m/z=374.32 (M+H⁺).

### Preparation of Compound 296

### N-methyl-4′-((2R,3S,4R,5S,6R)-3,4,5-trihydroxy-6-(hydroxymethyl)tetrahydro-2H-pyran-2-yl)-[1,1′-biphenyl]-4-carboxamide

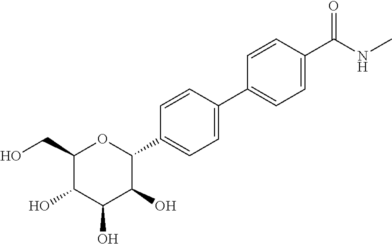 [SMILES: CNC(=O)c1ccc(-c2ccc([C@H]3O[C@H](CO)[C@@H](O)[C@H](O)[C@@H]3O)cc2)cc1]

The title compound is prepared following the procedure described in COMPOUND 158 using INTERMEDIATE O and 4-bromo-N-methylbenzamide as the appropriate starting materials.

¹H NMR (400 MHz, DMSO-D₆) δ 8.42 (d, J=4.6 Hz, 1H), 7.85 (d, J=8.3 Hz, 2H), 7.70 (d, J=8.3 Hz, 2H), 7.65 (d, J=8.2 Hz, 2H), 7.45 (t, J=9.8 Hz, 2H), 4.77 (d, J=4.8 Hz, 1H), 4.70 (m, 2H), 4.62 (d, J=5.9 Hz, 1H), 4.55 (t, J=5.8 Hz, 1H), 4.04 (m, 1H), 3.59 (t, J=5.6 Hz, 2H), 3.50 (m, 1H), 3.38 (m, 2H), 2.73 (d, J=4.4 Hz, 3H). LC-MS: m/z=374.32 (M+H⁺).

### Thermo Analysis of Compound 48

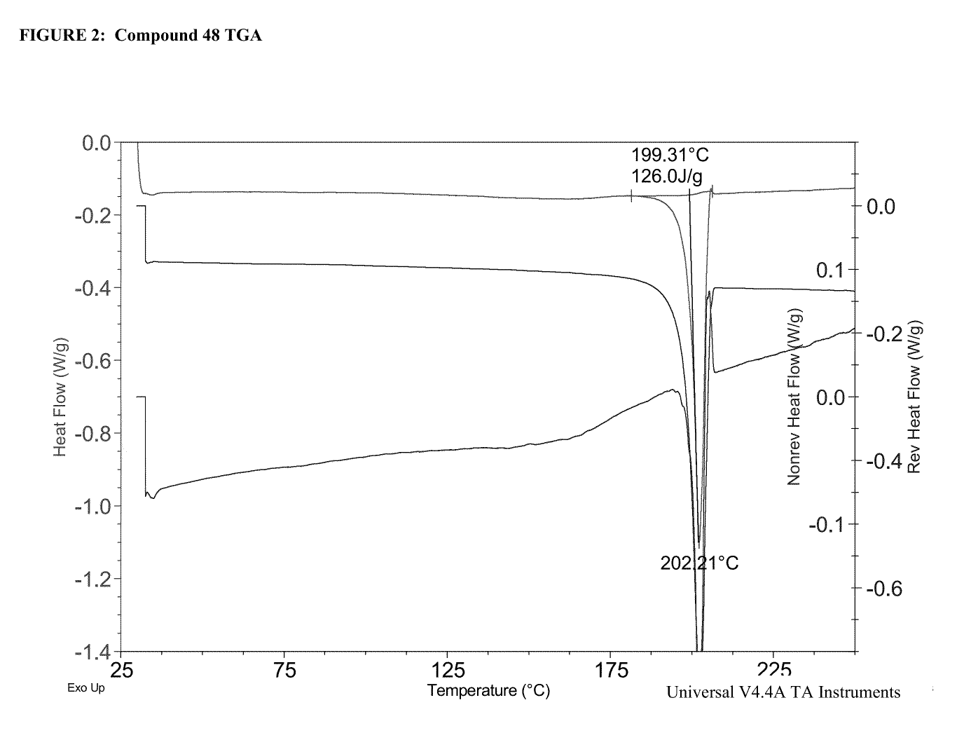

A thermal gravimetric analysis of Compound 48 was performed to determine the percent weight loss as a function of time using the TA Instrument TGA Q5000 (Asset V014258). A sample is added to a pre-tared aluminum pan and heated from ambient temperature to 350° C. at 10° C./min. Weight loss ca. 2.3% with gradual and significant weight loss observed at >100° C. The TGA result is shown in FIG. 2.

### XRPD of Compound 48 (Free Base)

The XRPD was recorded at room temperature in reflection mode using Bruker D8 Discover system (Asset Tag V012842) equipped with a sealed tube source and a Hi-Star area detector (Bruker AXS, Madison, Wis.). The X-Ray generator was operating at a tension of 40 kV and a current of 35 mA. The powder sample was placed on a Si zero-background wafer. Two frames were registered with an exposure time of 120 s each. The data were subsequently integrated over the range of 3°-41° 2q with a step size of 0.02° and merged into one continuous pattern. FIG. 1A shows the X-ray powder diffractogram of the sample. Representative XRPD peaks from Compound 48:

| No. | 2-Theta ° | Intensity % |
|---|---|---|
| 1 | 8.7 | 45.9 |
| 2 | 12.9 | 52.7 |
| 3 | 14.9 | 63.4 |
| 4 | 16.2 | 52.9 |
| 5 | 17.6 | 38.2 |
| 6 | 17.9 | 84.9 |
| 7 | 19.4 | 24.7 |
| 8 | 19.7 | 26.5 |
| 9 | 20.3 | 100 |
| 10 | 21.1 | 37.8 |
| 11 | 21.6 | 33.2 |
| 12 | 22.6 | 75.9 |
| 13 | 23.3 | 92.3 |
| 14 | 24.2 | 45.9 |
| 15 | 25.3 | 25.1 |
| 16 | 26.4 | 29 |
| 17 | 27.6 | 52.8 |
| 18 | 28.5 | 20.5 |

### Competitive Binding Assay

The first 177 amino acids of the FimH protein were expressed as a fusion protein with thrombin in a pET21b plasmid in bacteria. This FimH protein sequence contains the carbohydrate recognition domain (CRD) and shall be termed FimH-CRD. Following bacterial expression of the protein, the FimH-CRD protein was purified to homogeneity and the thrombin tag removed by protease cleavage. A competitive binding assay by fluorescence polarization was performed using 5 nM of the Alexa 647 mannoside probe and 60 nM of the FimH-CRD. The samples are assayed in a low volume 384 well microtiter plate in a final volume of 20 μl. The final assay buffer conditions are the following, 50 mM Tris-Cl, ph 7.0, 100 mM NaCl, 1 mM EDTA, 5 mM β-mercaptoethanol, 0.05% BSA and 2.5% DMSO. Two assays are performed for FimH, termed assay 1 or assay 2. The assay conditions are the same for both assays except the following: assay 1 has compounds prepared by manual dilution in a serial dilution factor with 12-point dose response while assay 2 has compounds prepared by a robotics system also through a serial dilution factor (12 point dose response) and initially prepared in duplicate in 384 well-Corning polypropylene round bottom plates. The assay 2 plates have compound which is then frozen and must be thawed prior to use. Initially the Alexa 647 probe and the FimH-CRD are added to the assay buffer and then 0.5 μl of test compound (assay 1 or 2) between 0.4 nM to 75 μM final concentration are added (12 point titration with 3-fold serial dilution). Control wells for the Alexa 647 probe are prepared with the same conditions except for the addition of the FimH-CRD protein. Plates are then incubated for 5 hrs at room temperature in the dark and under humid conditions to prevent drying. Plates are read using the SpectraMax Paradigm multi-mode plate reader and the appropriate fluorescent polarization detection cartridge (Alexa-647).

Alexa 647 mannoside probe is prepared using the similar procedure reported for FAM mannoside (Han, Z. et. al., 2010, J. Med. Chem., 53, 4779) and is described in the scheme below.

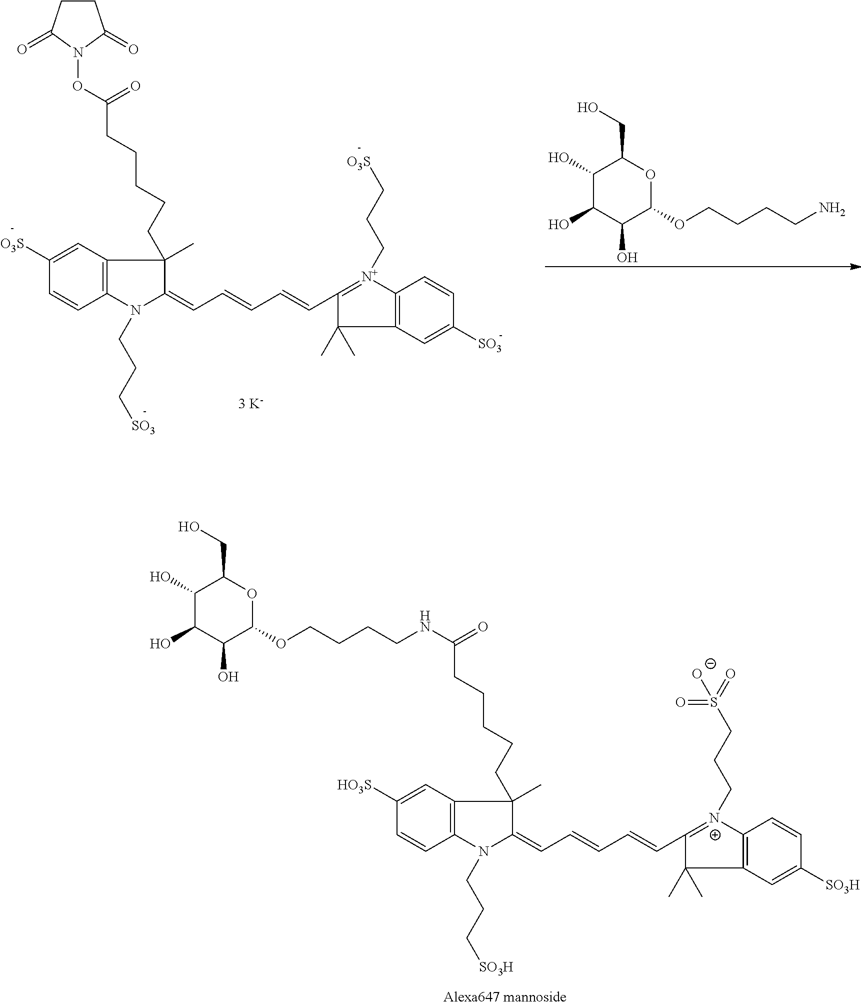

To a blue colored stirred solution of (2S,3S,4S,5S,6R)-2-(4-aminobutoxy)-6-(hydroxymethyl)tetrahydropyran-3,4,5-triol (2.21 mg, 0.009 mmol) and the (2E)-2-[(2E,4E)-5-[3,3-dimethyl-5-sulfonato-1-(3-sulfonatopropyl)indol-1-ium-2-yl]penta-2,4-dienylidene]-3-[6-(2,5-dioxopyrrolidin-1-yl)oxy-6-oxo-hexyl]-3-methyl-1-(3-sulfonatopropyl)indoline-5-sulfonate (Potassium Ion (3)) (4.9 mg, 0.0044 mmol) in DMF (44 μL) is added Et₃N (5.4 mg, 7.0 μL, 0.053 mmol) at RT. The solution is stirred at room temperature over night, concentrated, dissolved in water and purified on 12 g C-18 silica gel cartridge on Isolera system using acetonitrile in water (0 to 40%, 10 CV) and followed by lyophilization to afford Alexa 647 mannoside probe (3.3 mg, 34%) as deep blue solid.

The K_{d} values of the compounds are determined from dose response curves using twelve concentrations per compound in duplicate. Curves are fitted to data points using Fluorescence Polarization competitive displacement analysis, and Kds are interpolated from the resulting curves using GraphPad Prism software, version 50.4 (GraphPad software Inc., San Diego, Calif., USA).

TABLE 8

| | Assay 1 | Assay 2 | |
|---|---|---|---|
| | Kd (μM) ± Standard | Kd (μM) ± Standard | |
| | error of the mean | error of the mean | IC50 |
| COMPOUND | (number of repeats) | (number of repeats) | (μM) |
| 1 | 0.0123 ± 0.0051 (2) | | 0.053 |
| 2 | <0.005 (3) | | 0.029 |
| 3 | 0.0072 ± 0.0011 (4) | | 0.039 |
| 4 | 0.0051 ± 0.0001 (2) | 0.0136 ± 0.0061 (2) | 0.019 |
| 5 | <0.005 (2) | | 0.018 |
| 6 | 0.0137 ± 0.0077 (2) | | 0.087 |
| 7 | 0.0143 ± 0.0091 (3) | | 0.07 |
| 8 | 0.0401 ± 0.0171 (2) | | 0.155 |
| 9 | 0.0285 ± 0.0237 (3) | | 0.163 |
| 10 | 0.0134 ± 0.0066 (3) | 0.0101 ± 0.0006 (2) | 0.099 |
| 11 | 0.0119 ± 0.0073 (3) | | 0.088 |
| 12 | 0.0092 ± 0.0044 (2) | 0.0117 ± 0.0061 (2) | 0.067 |
| 13 | 0.0399 ± 0.0236 (2) | | 0.195 |
| 14 | 0.0778 ± 0.0704 (3) | | 0.551 |
| 15 | 0.0482 ± 0.0477 (3) | | 0.306 |
| 16 | 0.037 ± 0.0337 (3) | | 0.247 |
| 17 | 0.0086 ± 0.0025 (2) | | 0.048 |
| 18 | 0.0337 ± 0.0158 (2) | | 0.16 |
| 19 | 0.0062 ± 0.0012 (2) | | 0.052 |
| 20 | 0.0071 ± 0.0024 (2) | | 0.055 |
| 21 | 0.0123 ± 0.0051 (2) | | 0.092 |
| 22 | 0.0066 ± 0.0008 (2) | 0.0142 ± 0.0111 (2) | 0.049 |
| 23 | 0.0384 ± 0.0236 (2) | | 0.016 |
| 24 | 0.0567 ± 0.0002 (2) | | 0.243 |
| 25 | 0.007 ± 0.0005 (2) | | 0.036 |
| 26 | 0.0168 ± 0.0078 (2) | | 0.1 |
| 27 | 0.037 ± 0.0031 (2) | | 0.138 |
| 28 | 0.0167 ± 0.0075 (2) | | 0.056 |
| 29 | 0.0181 ± 0.0059 (2) | | 0.059 |
| 30 | 0.0098 ± 0.0018 (2) | | 0.053 |
| 31 | 0.0228 ± 0.0009 (2) | | 0.084 |
| 32 | 0.0226 ± 0.0187 (2) | | 0.045 |
| 33 | 0.0289 ± 0.0145 (2) | | 0.076 |
| 34 | 0.038 ± 0.0071 (2) | | 0.124 |
| 35 | 0.0562 ± 0.0049 (2) | 0.113 ± 0 (1) | 0.172 |
| 36 | 0.0532 ± 0.0065 (2) | | 0.168 |
| 37 | 0.0401 ± 0.0151 (3) | | |
| 38 | 0.0504 ± 0.0036 (2) | 0.0724 ± 0.0348 (2) | |
| 39 | 0.3957 ± 0.0447 (3) | | 1.395 |
| 40 | 0.4738 ± 0.0495 (2) | 0.4471 ± 0.4905 (2) | 2.05 |
| 41 | 0.0478 ± 0.0068 (2) | | 0.223 |
| 42 | 0.0584 ± 0.0098 (2) | | 0.225 |
| 43 | 0.0124 ± 0.0013 (2) | | 0.064 |
| 44 | 0.0418 ± 0.0116 (2) | 0.0504 ± 0.0101 (4) | 0.156 |
| 45 | 0.0769 ± 0.0491 (2) | | 0.183 |
| 46 | 0.0282 ± 0.0133 (2) | | 0.148 |
| 47 | 0.0406 ± 0.0104 (2) | | 0.163 |
| 48 | <0.005 (2) | 0.0085 ± 0.0013 (2) | 0.034 |
| 49 | 0.0063 ± 0.0014 (2) | | 0.045 |
| 50 | 0.0107 ± 0.0052 (2) | | 0.039 |
| 51 | 0.0052 ± 0.0001 (2) | | 0.033 |
| 52 | <0.005 (2) | | 0.023 |
| 53 | <0.005 (2) | 0.0157 ± 0.0102 (3) | 0.025 |
| 54 | <0.005 (2) | 0.0132 ± 0.0092 (3) | 0.016 |
| 55 | 0.0063 ± 0.0004 (2) | | 0.035 |
| 56 | 0.0054 ± 0.0003 (2) | | 0.014 |
| 57 | 0.0132 ± 0.0002 (2) | | 0.076 |
| 58 | 0.0111 ± 0.0016 (2) | | 0.066 |
| 59 | 0.0063 ± 0.0009 (2) | | 0.026 |
| 60 | 0.0087 ± 0.0021 (3) | 0.0107 ± 0.0053 (2) | 0.031 |
| 61 | 0.0172 ± 0.0107 (2) | | 0.042 |
| 62 | 0.0055 ± 0.0004 (2) | 0.0076 ± 0.002 (2) | |
| 63 | 0.0254 ± 0.0144 (2) | | 0.019 |
| 64 | 0.015 ± 0.0026 (2) | 0.0207 ± 0.0168 (3) | 0.055 |
| 65 | 0.0052 ± 0.0001 (2) | | 0.018 |
| 66 | 0.0143 ± 0.0066 (2) | | 0.024 |
| 67 | 0.021 ± 0.0043 (2) | | 0.106 |
| 68 | 0.0221 ± 0.0101 (3) | 0.035 ± 0.0212 (45) | 0.022 |
| 69 | 0.0131 ± 0.0007 (2) | | 0.066 |
| 70 | 0.0116 ± 0.0042 (2) | | 0.046 |
| 71 | 0.0146 ± 0.0023 (2) | | 0.067 |
| 72 | 0.0162 ± 0.0054 (2) | | 0.06 |
| 73 | | 0.0338 ± 0.0136 (2) | |
| 74 | | 0.0264 ± 0.0143 (2) | |
| 75 | | 0.04 ± 0.0113 (2) | |
| 76 | | 0.0389 ± 0.013 (2) | |
| 77 | | 0.0817 ± 0.011 (2) | |
| 79 | <0.005 (2) | 0.0128 ± 0.0009 (2) | |
| 80 | | 0.0123 ± 0.0062 (3) | |
| 81 | | 0.0152 ± 0.0043 (2) | |
| 82 | | 0.0232 ± 0.0151 (3) | |
| 83 | | 0.0322 ± 0.0058 (2) | |
| 84 | | 0.0275 ± 0.0039 (2) | |
| 85 | | <0.005 (1) | |
| 86 | | 0.0237 ± 0.0108 (3) | |
| 87 | | 0.0068 ± 0.0002 (2) | |
| 88 | | 0.0118 ± 0.0079 (2) | |
| 89 | | 0.0145 ± 0.0022 (3) | |
| 90 | | 0.0203 ± 0.0022 (2) | |
| 91 | | 0.0236 ± 0.0087 (2) | |
| 92 | 0.0249 ± 0.0113 (2) | | |
| 93 | | 0.0075 ± 0.0025 (2) | |
| 94 | | 0.0944 ± 0.013 (2) | |
| 95 | | 0.034 ± 0.0078 (2) | |
| 96 | | 0.029 ± 0.0023 (2) | |
| 97 | | 0.0316 ± 0.0175 (2) | |
| 98 | | 0.0278 ± 0.0021 (2) | |
| 99 | | 0.0264 ± 0.0011 (2) | |
| 100 | | 0.012 ± 0.0031 (3) | |
| 101 | | 0.0129 ± 0.0097 (2) | |
| 102 | | 0.022 ± 0.0034 (2) | |
| 103 | | 0.0097 ± 0.0045 (3) | |
| 104 | | 0.0085 ± 0.0031 (2) | |
| 105 | | 0.0281 ± 0.0039 (2) | |
| 106 | | 0.0234 ± 0.0075 (2) | |
| 107 | | 0.0098 ± 0.0006 (2) | |
| 108 | | 0.0079 ± 0.0029 (2) | |
| 109 | | 0.0144 ± 0.0062 (2) | |
| 110 | | 0.0289 ± 0.0045 (2) | |
| 113 | | 0.0207 ± 0.0047 (2) | |
| 115 | | 0.0242 ± 0.0029 (2) | |
| 117 | | 0.0209 ± 0.0067 (2) | |
| 118 | | 0.0131 ± 0.0002 (2) | |
| 119 | | 0.0195 ± 0.0088 (2) | |
| 120 | | 0.0128 ± 0.0021 (2) | |
| 121 | | 0.0131 ± 0.0009 (2) | |
| 122 | | 0.0276 ± 0.0074 (2) | |
| 123 | | 0.0371 ± 0.0085 (2) | |
| 124 | | 0.0162 ± 0.002 (2) | |
| 125 | | 0.0139 ± 0.0026 (2) | |
| 127 | | 0.0174 ± 0.0048 (2) | |
| 128 | | 0.0747 ± 0.0604 (2) | |
| 129 | | 0.0229 ± 0.0054 (2) | |
| 130 | <0.005 (2) | 0.0462 ± 0 (1) | |
| 131 | | 0.0228 ± 0.0113 (2) | |
| 132 | 0.0069 ± 0.00002 (2) | | |
| 133 | 0.0061 ± 0.0014 (2) | | |
| 134 | 0.0542 ± 0.017 (2) | | |
| 135 | | 0.0223 ± 0.0098 (2) | |
| 136 | | 0.0192 ± 0.0036 (2) | |
| 137 | | 0.0299 ± 0.0152 (2) | |
| 138 | | 0.0329 ± 0.0063 (2) | |
| 139 | <0.005 (1) | | |
| 140 | | 0.0153 ± 0.0035 (2) | |
| 141 | | 0.0562 ± 0.02 (2) | |
| 142 | | 0.0148 ± 0.0059 (2) | |
| 143 | | 0.0112 ± 0.001 (2) | |
| 144 | | 0.0183 ± 0.0075 (2) | |
| 145 | | 0.0224 ± 0.0047 (2) | |
| 146 | | 0.0278 ± 0.0108 (2) | |
| 147 | | 0.0109 ± 0.004 (2) | |
| 148 | | 0.0251 ± 0.012 (2) | |
| 149 | | 0.0198 ± 0.0112 (2) | |
| 150 | | 0.0259 ± 0.0051 (2) | |
| 151 | | 0.0378 ± 0.0134 (2) | |
| 152 | | 0.0438 ± 0.0011 (2) | |
| 153 | | 0.0887 ± 0.0412 (2) | |
| 154 | | 0.0184 ± 0.0069 (2) | |
| 155 | | 0.0717 ± 0.002 (2) | |
| 156 | | 0.0165 ± 0.0043 (2) | |
| 157 | | 0.0343 ± 0.0081 (2) | |
| 158 | | 0.0205 ± 0.0043 (2) | |
| 159 | | 0.0156 ± 0.0034 (2) | |
| 161 | | 0.0066 ± 0.0012 (2) | |
| 162 | | 0.0395 ± 0.011 (2) | |
| 163 | | 0.0821 ± 0.0006 (2) | |
| 164 | | 0.0502 ± 0.0141 (2) | |
| 165 | | 0.0289 ± 0.0117 (2) | |
| 166 | | 0.0441 ± 0.0199 (3) | |
| 167 | | 0.0321 ± 0.0043 (2) | |
| 168 | | 0.0425 ± 0.0183 (2) | |
| 169 | | 0.0257 ± 0.0004 (2) | |
| 170 | | 0.0324 ± 0.0144 (2) | |
| 171 | | 0.0321 ± 0.0191 (2) | |
| 172 | | 0.5299 ± 0.7142 (2) | |
| 173 | | 0.0546 ± 0.023 (2) | |
| 174 | | 0.0136 ± 0.0034 (2) | |
| 175 | | 0.0102 ± 0.00004 (2) | |
| 176 | | 0.0211 ± 0.0026 (2) | |
| 177 | | 0.0121 ± 0.0058 (2) | |
| 178 | | 0.0179 ± 0.0037 (2) | |
| 179 | | 0.0204 ± 0.0008 (2) | |
| 181 | | 0.0217 ± 0.0042 (2) | |
| 182 | | 0.0202 ± 0.0047 (2) | |
| 183 | | 0.0248 ± 0.0083 (2) | |
| 184 | | 0.0182 ± 0.0084 (2) | |
| 185 | | 0.0117 ± 0.0075 (2) | |
| 186 | | 0.0123 ± 0.0089 (3) | |
| 187 | | 0.0099 ± 0.0019 (4) | |
| 188 | | 0.0111 ± 0.0011 (3) | |
| 189 | | 0.0081 ± 0.0027 (3) | |
| 190 | | 0.0241 ± 0.0153 (2) | |
| 191 | | 0.0374 ± 0.0172 (2) | |
| 192 | | 0.0565 ± 0.0091 (2) | |
| 193 | | 0.0306 ± 0.0011 (2) | |
| 194 | | 0.031 ± 0.0294 (2) | |
| 195 | | 0.1784 ± 0.0609 (2) | |
| 196 | | 0.0219 ± 0.0018 (2) | |
| 197 | | 0.0117 ± 0.0028 (2) | |
| 198 | | 0.0108 ± 0.0052 (2) | |
| 199 | | 0.0083 ± 0.0022 (2) | |
| 200 | | 0.0084 ± 0.0008 (2) | |
| 201 | | 0.1027 ± 0.0018 (2) | |
| 202 | | 0.0192 ± 0.0038 (2) | |
| 203 | | 0.0092 ± 0.0012 (2) | |
| 204 | | 0.0106 ± 0.0009 (2) | |
| 205 | | 0.0387 ± 0.0162 (2) | |
| 206 | | 0.0284 ± 0.0062 (2) | |
| 207 | | 0.0094 ± 0.0043 (2) | |
| 209 | | 0.0125 ± 0.0053 (2) | |
| 210 | | 0.0242 ± 0.0013 (2) | |
| 211 | | 0.008 ± 0.0024 (2) | |
| 212 | | 0.0189 ± 0.0008 (2) | |
| 213 | | 0.0093 ± 0.0005 (2) | |
| 214 | | 0.012 ± 0.0079 (2) | |
| 215 | | 0.0131 ± 0.0039 (3) | |
| 216 | | 0.0164 ± 0.0052 (3) | |
| 217 | | 0.0121 ± 0.0076 (2) | |
| 218 | | 0.0087 ± 0.0014 (2) | |
| 219 | | 0.0163 ± 0.0051 (2) | |
| 220 | | 0.0166 ± 0.0044 (2) | |
| 221 | | 0.0082 ± 0.0005 (2) | |
| 222 | | 0.0407 ± 0.0012 (2) | |
| 223 | | 0.0232 ± 0.0016 (2) | |
| 225 | | 0.0345 ± 0.0123 (2) | |
| 226 | | 0.0321 ± 0.0089 (2) | |
| 227 | | 0.0371 ± 0.0154 (2) | |
| 228 | | 0.0092 ± 0.00004 (2) | |
| 229 | | 0.0331 ± 0.004 (2) | |
| 230 | | 0.0139 ± 0.0036 (2) | |
| 231 | | 0.0089 ± 0.0014 (2) | |
| 232 | | 0.0085 ± 0.0042 (2) | |
| 233 | | 0.0103 ± 0.0039 (2) | |
| 234 | | 0.0114 ± 0.0045 (2) | |
| 235 | | 0.0141 ± 0.0003 (2) | |
| 236 | | 0.0115 ± 0.0046 (2) | |
| 237 | | 0.0079 ± 0.0016 (2) | |
| 238 | | 0.0097 ± 0 (1) | |
| 239 | | 0.0077 ± 0.00005 (2) | |
| 240 | | 0.0076 ± 0.0009 (2) | |
| 241 | | 0.0102 ± 0.0008 (2) | |
| 242 | | 0.0215 ± 0.0039 (2) | |
| 243 | | 0.0058 ± 0 (1) | |
| 244 | | 0.0233 ± 0.0011 (2) | |
| 245 | | 0.0228 ± 0.0024 (2) | |
| 246 | | 0.0236 ± 0.0055 (2) | |
| 247 | | 0.032 ± 0.0051 (2) | |
| 248 | 0.014 ± 0.0084 (2) | | |
| 249 | | 0.0196 ± 0.0102 (3) | |
| 250 | | 0.0406 ± 0.0234 (2) | |
| 251 | | 0.0299 ± 0.0139 (3) | |
| 252 | | 0.0132 ± 0.0022 (2) | |
| 253 | | <0.005 (2) | |
| 254 | | 0.0079 ± 0 (1) | |
| 255 | 0.0087 ± 0.002 (2) | | |
| 256 | | 0.0676 ± 0.0218 (2) | |
| 257 | | 0.0192 ± 0.0063 (2) | |
| 258 | | 0.0319 ± 0.0063 (2) | |
| 259 | | | |
| 260 | | 0.1251 ± 0.0526 (2) | |
| 261 | | 0.0322 ± 0.0081 (2) | |
| 262 | | 0.0266 ± 0.0114 (2) | |
| 263 | | 0.0223 ± 0.005 (2) | |
| 264 | | 0.0308 ± 0.0091 (2) | |
| 265 | 0.0521 ± 0.0138 (2) | | 0.162 |
| 266 | 0.0213 ± 0.0062 (2) | | 0.059 |
| 267 | 0.0128 ± 0.008 (3) | | 0.068 |
| 268 | 0.019 ± 0.0038 (2) | | 0.077 |
| 269 | <0.005 (2) | | 0.010 |
| 270 | 0.0144 ± 0.0026 (2) | | 0.054 |
| 271 | 0.0147 ± 0.007 (3) | | 0.027 |
| 272 | 0.0127 ± 0.0077 (2) | | 0.046 |
| 273 | 0.0121 ± 0.0057 (3) | 0.0218 ± 0.0052 (2) | 0.038 |
| 274 | 0.0431 ± 0.053 (2) | | 0.034 |
| 275 | 0.0268 ± 0.0154 (2) | | 0.028 |
| 276 | 0.0111 ± 0.0034 (2) | | 0.049 |
| 277 | 0.0107 ± 0.0039 (2) | | 0.047 |
| 278 | 0.0127 ± 0.0009 (2) | | 0.064 |
| 279 | 0.0099 ± 0.0011 (2) | | 0.051 |
| 280 | 0.0181 ± 0.0071 (2) | | 0.059 |
| 281 | 0.0511 ± 0.0076 (2) | | 0.216 |
| 282 | 0.0406 ± 0.00003 (2) | | 0.212 |
| 283 | 0.0108 ± 0.0041 (2) | | 0.027 |
| 284 | 0.166 ± 0.0965 (3) | | 0.049 |
| 285 | 0.0147 ± 0.0012 (2) | | 0.080 |
| 286 | 0.0174 ± 0.0048 (2) | 0.0351 ± 0.0167 (2) | 0.063 |
| 287 | 0.0322 ± 0.0085 (2) | | 0.107 |
| 288 | 0.0287 ± 0.0256 (2) | | 0.046 |
| 289 | 0.1619 ± 0.241 (4) | | 0.175 |
| 290 | 0.0119 ± 0.0049 (2) | | 0.029 |
| 292 | 0.006 ± 0.0004 (2) | — | |
| 293 | 0.009 ± 0.002 (2) | | |
| 294 | 0.011 ± 0.003 (2) | | |
| 295 | 0.012 ± 0.003 (2) | | |
| 296 | 0.005 ± 0.0004 (2) | | |
| Intermediate C | 0.024 ± 0.005 (2) | 0.027 (1) | |

### Mouse Model of Inflammatory Bowel Disease (IBD):

Transgenic humanized-CEACAM6 mice model can be used. (Carvalho F A et al. (2009) J Exp Med. September 28; 206(10):2179-89). The Transgenic humanized-CEACAM6 mice are infected as described. The infected mice can be then treated with COMPOUNDS of the present invention.

While we have described a number of embodiments of this invention, it is apparent that our basic examples may be altered to provide other embodiments that utilize the COMPOUNDS, methods, and processes of this invention. Therefore, it will be appreciated that the scope of this invention is to be defined by the appended claims rather than by the specific embodiments that have been represented by way of example herein.

1. A compound of Formula (I), or a pharmaceutically acceptable salt thereof: 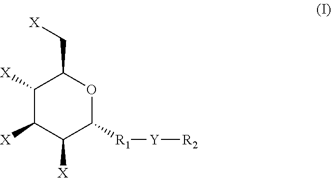
wherein
X is —OR₇;
Y is absent or a C₁-C₁₀ aliphatic wherein up to four methylene units of the C₁-C₁₀ aliphatic can be optionally replaced with —NR₈, —O—, —S—, C(O)—, —S(O)—, or —SO₂—; Y is optionally substituted with 1-2 occurrences of halogen, OH, C₃₋₆cycloalkyl or C₁₋₆aliphatic;
R₁ is C₆₋₁₀ aryl optionally substituted with one or more R_{3A} groups; and
R₂ is H, C₃-C₆ cycloalkyl, 3-8 membered heterocyclyl, C₆₋₁₀ aryl, (C₆₋₁₀ aryl)-(C₁-C₆alkyl)-, or 5-10 membered heteroaryl; each R₂ is independently and optionally substituted with one or more R_{3B} groups and optionally substituted with one R₃ group;
each R_{3A} and R_{3B} is independently halogen, —CN, NO₂, C₃-C₆ cycloalkyl, 3-8 membered heterocyclyl, (C₆₋₁₀ aryl)-(C₁-C₆alkyl)-; or a C₁-C₁₀ aliphatic wherein up to four methylene units of the C₁-C₁₀ aliphatic can be optionally replaced with —NR₄, —O—, —S—, —C(O)—, —S(O)—, —SO₂—, or —P(O)—; each R_{3A} and R_{3B} is independently and optionally substituted with one or more R₄ or R_{4A} groups;
R₃ is C₃-C₆ cycloalkyl, 3-8 membered heterocyclyl, C₆₋₁₀ aryl, (C₆₋₁₀ aryl)-(C₁-C₆alkyl)-, or 5-10 membered heteroaryl; each R₃ is optionally substituted with one or more R₄ or R_{4A} groups;
R₄ is H, C₁-C₆ alkyl, C₁-C₆ alkenyl, C₁-C₆ alkynyl, C₃₋₈ cycloalkyl, 3-8 membered heterocyclyl, C₆₋₁₀ aryl or 5-10 membered heteroaryl; each R₄ is optionally substituted with one or more R_{4B} groups;
R_{4A} is halogen, CN, NO₂, or a C₁-C₁₀ aliphatic wherein up to four methylene units of the C₁-C₁₀ aliphatic can be optionally replaced with —NR₄, —O—, —S—, —C(O)—, —S(O)—, —SO₂—, or —P(O)—; each R_{4A} is optionally substituted with 0-3 halo;
R_{4B} is halogen, CN, NO₂, or a C₁-C₁₀ aliphatic wherein up to four methylene units of the C₁-C₁₀ aliphatic can be optionally replaced with —NR, —O—, —S—, —C(O)—, —S(O)—, —SO₂—, or —P(O)—; each R_{4A} is optionally substituted with 0-3 halo;
R₇ is H or a 5-6 membered heterocyclyl having 1-2 heteroatoms selected from oxygen, nitrogen, or sulfur; wherein said 5-6 membered heterocyclyl is independently and optionally substituted with 1-4 occurrences of C₁₋₄alkyl wherein up to one methylene unit of the C₁₋₄alkyl is optionally replaced with —O—;
R₈ is H, C₁-C₆ alkyl, C₁-C₆ alkenyl, C₁-C₆ alkynyl, C₃-C₆ cycloalkyl, 3-8 membered heterocyclyl, C₆₋₁₀ aryl or 5-10 membered heteroaryl; or —C(O)R₉;
R₉ and R₁₀ are each independently C₁-C₆ alkyl or C₃-C₆ cycloalkyl;
R is H, C₁-C₆ alkyl or C₃-C₆ cycloalkyl;
m is 0, 1 or 2; and
n is 0, 1, 2, 3, or 4;
provided that the compound is not one of the following:

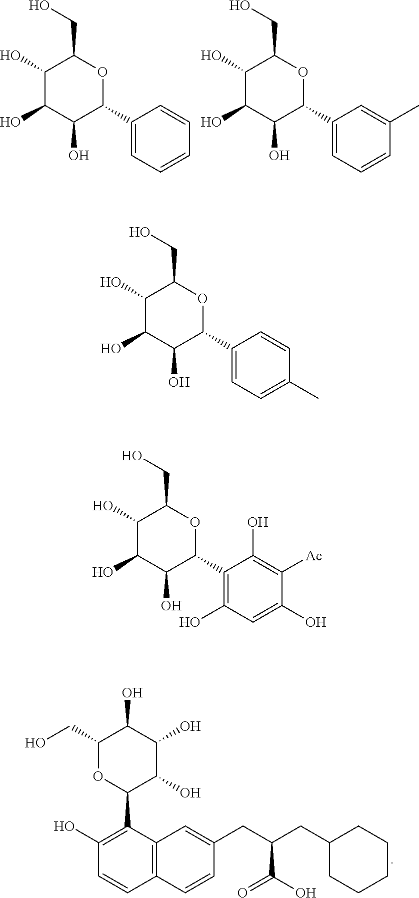

2. The compound of claim 1, wherein
Y is absent, or is —NR₈, —O—, —S—, —C(O)—, —C(R¹⁰)(OH)—, —C(O)N(R₈)(CH₂)ₘ—, —N(R₈)C(O)O—, —OC(O)NR₈—, —NR₈SO₂—, —NR₈—C(O)—, —SO₂—, —NR₈C(O)NR₈—, —S(O)—, —SO₂NR₈, —(C₁-C₆)alkyl-, —(C₁-C₆)alkenyl-, —(C₁-C₆)alkynyl-, —(O—(C₁-C₆ alkyl))ₙ-, —O—(C₁₋₆alkyl)NR₈C(O)—, —O—(C₁₋₆alkyl)C(O)NR₈, —O—(C₁₋₆alkyl)-C(O)—, or —((C₁-C₆)alkyl)-O—;
each R_{3A} and R_{3B} is independently —OH, —CN, halogen, —C(R₁₀)₃, —C(R₁₀)₂OH, —(CH₂)ₙOR₄, —(CH₂)ₙC(O)OR₄, —(CH₂)ₙN(R₄)₂, —C(O)OR₄, —C(O)N(R₄)₂, —N(R₄)C(O)(R₄)₂, —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, or —P(O)(R₄)₂; and
R_{4A} is —OH, —CN, halogen, —C(R₁₀)₃, —C(R₁₀)₂OH, —(CH₂)ₙOR₄, —(CH₂)ₙC(O)OR₄, —(CH₂)ₙN(R₄)₂, —C(O)OR₄, —C(O)N(R₄)₂, —N(R₄)C(O)(R₄)₂, —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —P(O)(OR₄)₂, —P(O)(R₄)₂; and
R⁷ is H or mannosyl.

3. The compound of claim 1, wherein:
X is —OH;
Y is absent, or is —NR₈, —O—, —S—, —C(O)—, —C(R¹⁰)(OH)—, —SO₂—, —S(O)—, —(C₁-C₆)alkyl, —(C₁-C₆)alkenyl, —(C₁-C₆)alkynyl, —(O—(C₁-C₆ alkyl))ₙ-, —O(C₁₋₆alkyl)N—R₈C(O)—, —O—(C₁₋₆alkyl)-C(O)NR₈, —O—(C₁₋₆alkyl)C(O)—, or —((C₁-C₆)alkyl)-O—;
R₂ is C₆₋₁₀ aryl, (C₆₋₁₀ aryl)-(C₁-C₆alkyl)-, or 5-10 membered heteroaryl; each R₂ is independently and optionally substituted with one or C₆₋₁₀ aryl, (C₆₋₁₀ aryl)-(C₁-C₆ alkyl)-, or 5-10 membered heteroaryl;
R₈ is —H, C₁-C₆ alkyl, C₁-C₆ alkenyl, C₁-C₆ alkynyl, or C₃-C₆ cycloalkyl.

4. The compound of claim 3 wherein R₁ is optionally substituted phenyl.

5. The compound of claim 3 wherein R₁ is optionally substituted naphthyl.

6. The compound of claim 1 wherein X is —OR₇ and R₇ is H or

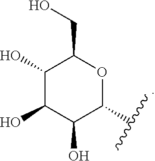 [SMILES: *[C@H]1O[C@H](CO)[C@@H](O)[C@H](O)[C@@H]1O ; "."]

7. The compound of claim 6, wherein R₇ is bonded as shown in Formula IA, IB, IC, or ID: 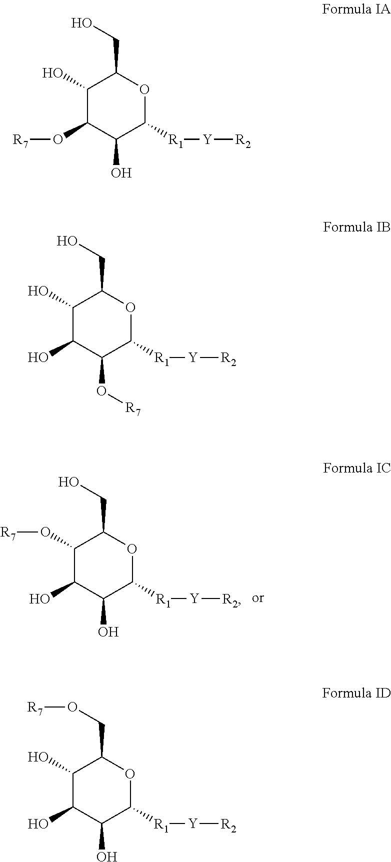
wherein R₁, Y, and R₂ are as defined in any one of claims 1-7.

8. The compound of claim 6, wherein R₇ is H.

9. The compound of claim 1, wherein X is —OH, —F, —OCH₃, or —CH₃.

10. The compound of claim 1 wherein:
X is —OR₇ and R₇ is H or 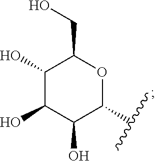 [SMILES: *[C@H]1O[C@H](CO)[C@@H](O)[C@H](O)[C@@H]1O ; ";"]
R₁ is phenyl or naphthyl;
Y is absent, or is —O—, —C(O)N(R₈)(CH₂)ₘ—, —OC(O)NR₈—, —(C₁-C₆)alkyl-, —(C₁-C₆)alkenyl-, —(C₁-C₆)alkynyl-, —(O—(C₁-C₆alkyl)ₙ-, —O(C₁₋₆alkyl)NR₈C(O)—, —O(C₁₋₆alkyl)C(O)NR₈, —O(C₁₋₆alkyl)C(O)—, or —((C₁-C₆)alkyl)-O—;
R₂ is C₆₋₁₀aryl, a 5-6 membered monocyclic heteroaryl having 1-4 heteroatoms selected from oxygen, nitrogen, or sulfur; or an 8-10 membered bicyclic heteroaryl having 1-4 heteroatoms selected from oxygen, nitrogen, or sulfur; a 3-8 membered monocyclic heterocyclyl having 1-2 heteroatoms selected from oxygen, nitrogen, or sulfur; or a C₃₋₆cycloalkyl;
R₃ is phenyl or a 5-6 membered monocyclic heteroaryl having 1-4 heteroatoms selected from oxygen, nitrogen, or sulfur.

11. The compound of claim 1 wherein:
X is —OR₇ and R₇ is H or 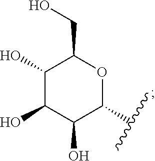 [SMILES: *[C@H]1O[C@H](CO)[C@@H](O)[C@H](O)[C@@H]1O ; ";"]
R₁ is phenyl or naphthyl;
Y is absent, or is —O—, —C(O)N(R₈)(CH₂)ₘ—, —OC(O)NR₈—, —(C₁-C₆)alkyl-, —(C₁-C₆)alkenyl-, —(C₁-C₆)alkynyl-, —(O—(C₁-C₆alkyl)ₙ-, —O(C₁₋₆alkyl)NR₈C(O)—, —O(C₁₋₆alkyl)C(O)NR₈, —O(C₁₋₆alkyl)C(O)—, or —((C₁-C₆)alkyl)-O—;
R₂ is phenyl, naphthyl, imidazolyl, pyrazolyl, triazolyl, thienyl, thiadiazolyl, thiazolyl, oxadiazolyl, pyridinyl, pyrimidinyl, benzodioxolyl, indolyl, benzimidazolyl, benzothiazolyl, benzooxadiazolyl, imidazopyridinyl, quinolinyl, oxetanyl, tetrahydropyranyl, and C₃₋₆cycloalkyl; and
R³ is phenyl, pyrazolyl, triazolyl, oxazolyl, isoxazolyl, oxadiazolyl, thiazolyl, thiadiazolyl, pyridinyl.

12. The compound of claim 1 wherein:
X is —OH;
Y is absent, or is —O—, —S—, —OC(O)NR₈—, or —C(O)N(R₄)(CH₂)ₘ—;
R₁ is aryl optionally substituted with one or more R_{3A} and groups; and
R₂ is —H, or alkyl, cycloalkyl, heterocycle, aryl, aralkyl, or heteroaryl; each optionally substituted with one or more R_{3B} groups and optionally one R₃;
wherein each R_{3A} and R_{3B} is independently —OH, —CN, halogen, —C(R₁₀)₃, —(CH₂)ₙOR₄, —(CH₂)ₙC(O)OR₄, —(CH₂)ₙN(R₄)₂, —C(O)R₄, —C(O)N(R₄)₂, —N(R₄)C(O)(R₄)₂, —OC(O)NHR₄, —NHC(O)OR₄, —NHSO₂R₄, —NH—C(O)R₄, —SO₂—R₄, —NHC(O)NHR₄, —S(O)R₄, —SO₂NHR₄, —SR₄, —C₁-C₆ alkyl, aryl, aralkyl, or heteroaryl; each optionally substituted with one or more R₄ groups;
wherein each R₄ is independently —H or C₁-C₆ alkyl;
wherein m is 0, 1 or 2; and
wherein n is 0, 1, or 2,
or a pharmaceutically acceptable salt thereof.

13. The compound of claim 12 wherein
X is —OH;
Y is absent;
R₁ is phenyl optionally substituted with one or more halogen, —OR₄, or —(CH₂)ₙC(O)OR₄;
R₂ is heteroaryl optionally substituted with one or more R_{3B} groups;
R_{3B} is C₁-C₆ alkyl or C(R₁₀)₃; and
R₄ is H or C₁-C₆ alkyl.

14. The compound of claim 12 wherein
X is —OH;
Y is absent;
R₁ is phenyl optionally substituted with one or more halogen, —OR₄, or —(CH₂)ₙC(O)OR₄;
R₂ is aryl optionally substituted with one or more R_{3B} groups;
R_{3B} is —OH, halogen, —CN, —(CH₂)ₙC(O)OR₄, —(CH₂)ₙOR₄, —(CH₂)ₙN(R₄)₂, —C(O)NHR₄, —NH—C(O)R₄, —SO₂R₄, or —C(O)OR₄; and
R₄ is H or C₁-C₆ alkyl.

15. The compound of claim 10 wherein X is —OH.

16. The compound of claim 12 or a pharmaceutically acceptable salt thereof, wherein Y is —C(O)N(R₄)(CH₂)ₘ—; R₁ is optionally substituted phenyl; R₂ is C₁-C₆ alkyl, cycloalkyl, aralkyl; a heteroaryl ring; or an aryl ring optionally substituted with one or more R_{3B} groups and optionally one R₃; wherein the heteroaryl ring is selected from the group consisting of: pyrazole, thiadiazole, quinoline, indole, thiazole, pyridine and benzothiazole and wherein R_{3A} and R_{3B} are each independently halogen, C₁-C₆ alkyl, or benzyl.

17-24. (canceled)

25. The compound of claim 12 or a pharmaceutically acceptable salt thereof, wherein Y is −OC(O)NR₈—; R₁ is optionally substituted phenyl; R₂ is phenyl, benzyl, or thiophenyl optionally substituted with one or more R_{3B} groups and optionally one R₃ wherein R_{3B} is halogen, C₁-C₆ alkyl, or —N(R₄)₂.

26-30. (canceled)

31. The compound of claim 12 or a pharmaceutically acceptable salt thereof, wherein Y is C₁-C₆ alkyl, C₁-C₆ alkenyl, or C₁-C₆ alkynyl.

32. The compound of claim 12 or a pharmaceutically acceptable salt thereof, wherein Y is absent.

33. The compound of claim 32, or a pharmaceutically acceptable salt thereof, wherein R₁ is optionally substituted phenyl.

34. The compound of claim 33, or a pharmaceutically acceptable salt thereof, wherein R₁ is phenyl substituted with one or more R_{3A}, wherein R_{3A} is halogen, C₁-C₆alkyl, C₁-C₆alkenyl, C₁-C₆alknyl, or a C₁-C₁₀ aliphatic wherein up to four methylene units of the C₁-C₁₀ aliphatic can be optionally replaced with —NR₄, —O—, or —C(O)—.

35. The compound of claim 33, or a pharmaceutically acceptable salt thereof, wherein R₁ is phenyl substituted with one or more halogen, —OR₄, or —(CH₂)ₙC(O)OR₄.

36. The compound of claim 33, or a pharmaceutically acceptable salt thereof, wherein R₁ is phenyl substituted with one or more halogen, —O(C₁-C₆alkyl), or C₁-C₆alkyl.

37. The compound of claim 34, or a pharmaceutically acceptable salt thereof, wherein R₄ is —H or C₁-C₆ alkyl.

38. The compound of claim 34, or a pharmaceutically acceptable salt thereof, wherein R₁ is phenyl substituted with one or more R_{3A}, wherein R_{3A} is fluoro, bromo, chloro, CH₃, CH₂CH₃, —C≡CH, OH, OCH₃, OCF₃, —OCH₂C(CH₃)₃, —O(CH₂)₄CF₃, —OCH₂C(O)NHCH₃—, —OCH₂C(O)OCH₃, —OCH₂C≡CCH₂CH₃, —O(CH₂)₃CN,
—OCH₂CH(CH₃)CH₂CH₃, —OCH₂CH₂CH(CH₃)₂, —O(CH₂)₃OCH₃, —O(CH₂)₂F, —O(CH₂)₃F, or —CH₂CH₂C(O)OCH₃.

39. The compound of claim 33, or a pharmaceutically acceptable salt thereof, wherein R₂ is a heteroaryl ring optionally substituted with one or more R_{3B} groups and optionally one R₃.

40. The compound of claim 39, or a pharmaceutically acceptable salt thereof, wherein the heteroaryl ring is imidazolyl, pyrazolyl, triazolyl, thienyl, thiadiazolyl, thiazolyl, oxadiazolyl, pyridinyl, pyrimidinyl, benzodioxolyl, indolyl, benzimidazolyl, benzothiazolyl, benzooxadiazolyl, imidazopyridinyl, quinolinyl, oxetanyl, tetrahydropyranyl, and C₃₋₆cycloalkyl;

41. The compound of claim 39, or a pharmaceutically acceptable salt thereof, wherein the heteroaryl ring is selected from the group consisting of: pyrimidinyl, benzodioxolyl, benzodioxanyl, benzothiophenyl, indolyl, pyrazolyl, and benzimidazolyl.

42. The compound of claim 39 as represented by the following formula: 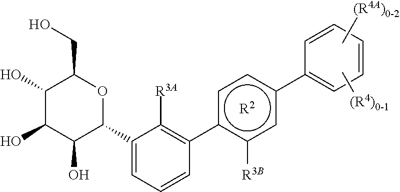
wherein R^{3A}, R^{3B}, and R^{4A} are each independently halogen, —O(C₁-C₆alkyl), or C₁-C₆alkyl; and R² is a 6-membered aryl or heteroaryl ring.

43. The compound of claim 39 or a pharmaceutically acceptable salt thereof, wherein R_{3B} is halogen, CN, NO₂, or a C₁₋₆ aliphatic wherein up to four methylene units of the C₁₋₆ aliphatic can be optionally replaced with —NR₄, —O—, —C(O)— or —S(O)₂—, wherein R_{3B} is optionally substituted with one or more halogen.

44. The compound of claim 39 or a pharmaceutically acceptable salt thereof, wherein R_{3B} is fluoro, chloro, CN, NO₂, NH₂, CH₃, CF₃, C(O)CH₃, C(O)NH(CH₃), CH₂OH, OH, butyl, CH₂C(O)NHCH₃, S(O)₂CH₃,

45. The compound of claim 39 or a pharmaceutically acceptable salt thereof, wherein R_{3B} is C₁-C₆ alkyl, or —C(R₁₀)₃.

46. The compound of claim 33, or a pharmaceutically acceptable salt thereof, wherein R₂ is aryl optionally substituted with one or more R_{3B} groups and optionally one R₃.

47. The compound of claim 46, or a pharmaceutically acceptable salt thereof, wherein R₂ is phenyl or naphthalene each optionally substituted with one or more R_{3B} groups and optionally one R₃.

48. The compound of claim 47, or a pharmaceutically acceptable salt thereof, wherein R_{3B} is halogen, CN, NO₂, or a C₁₋₆ aliphatic wherein up to four methylene units of the C₁₋₆ aliphatic can be optionally replaced with —NR₄, —O—, —C(O)— or —S(O)₂—, wherein R_{3B} is optionally substituted with one or more halogen.

49. The compound of claim 47, or a pharmaceutically acceptable salt thereof, wherein R_{3B} is fluoro, chloro, CN, CH₃, CH₂CH₃, CH₂CH₂CH₃, C(CH₃)₃, C(O)CH₃, CH₂C(O)OCH₃, C(O)OH, C(O)OCH₃, C(O)NHCH₃, NHC(O)CH₃, NHC(O)CHC(CH₃)₂, CH₂OH, CH₂OCH₃, CH₂N(CH₃)₂, NH₂, N(CH₃)₂, OH, OCH₃, O(CH₂)₂CH₃, S(O)₂NHCH₃, or S(O)₂CH₃.

50. The compound of claim 47, or a pharmaceutically acceptable salt thereof, wherein R_{3B} is —OH, halogen, —CN, —OR₄, —(CH₂)ₙC(O)OR₄, —(CH₂)ₙOR₄, —(CH₂)ₙN(R₄)₂, —C(O)NHR₄, —NH—C(O)R₄, —SO₂R₄, or —C(O)OR₄.

51. The compound of claim 47, or a pharmaceutically acceptable salt thereof, wherein R₂ is substituted with one occurrence of R₃, wherein R₃ is a heteroaryl ring optionally substituted with one or more R₄ or R_{4A} groups.

52. The compound of claim 51, or a pharmaceutically acceptable salt thereof, wherein the heteroaryl ring is oxadiazolyl.

53. The compound of claim 52, or a pharmaceutically acceptable salt thereof, wherein R₄ is —H or C₁-C₆ alkyl.

54. The compound of claim 52, wherein R₁ is phenyl and R₂ is phenyl.

55. The compound of claim 33, or a pharmaceutically acceptable salt thereof, wherein R₂ is —H.

56. The compound of claim 12, or a pharmaceutically acceptable salt thereof, wherein Y is —O—; R₁ is optionally substituted phenyl; R_{3A} is halogen, C₁-C₆alkyl, or —O(C₁-C₆alkyl); R₂ is phenyl optionally substituted with one or more R_{3B} groups.

57-60. (canceled)

61. The compound of claim 47, wherein R³ is R^{3A}, R^{3B}, or R^{3C} as represented by the following formula: 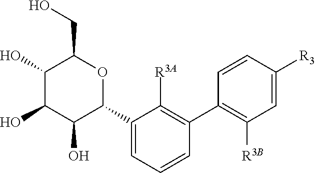
wherein R^{3A} and R^{3B} are each independently halogen, C₁-C₆alkyl, or —O(C₁-C₆alkyl); and R₃ is a heteroaryl ring optionally substituted with one or more R₄ groups.

62. The compound of claim 61, wherein R₃ is a 5-membered heteroaryl.

63. The compound of claim 62, wherein R₃ is oxadiazolyl, pyrazolyl, or thiadiazolyl.

64. The compound of claim 63, wherein R₃ is oxadiazolyl.

65. The compound of claim 61, or a pharmaceutically acceptable salt thereof, wherein R_{3B} is halogen, C₁-C₆ alkyl, —(CH₂)ₙC(O)OR₄, or —C(O)NHR₄.

66. The compound of claim 61, or a pharmaceutically acceptable salt thereof, wherein R_{3B} is halogen, C₁-C₆alkyl, —O(C₁-C₆alkyl).

67. The compound of claim 61, or a pharmaceutically acceptable salt thereof, wherein R₃ is a heteroaryl ring optionally substituted with one or more R_{4A} or R₄ groups.

68. The compound of claim 67, or a pharmaceutically acceptable salt thereof, wherein R_{4A} is CH₃, C(O)CH₃,
C(O)NHCH₃, —CH₂N(CH₃)₂, CH₂C(O)OH, —CH₂C(O)NHCH₃, NHC(O)O(CH₃)₃, CH(CH₃)₂, or CH₂C(O)NH(CH₂CH₂O)₂CH₃ and R₄ is pyridinyl or furanyl.

69. The compound of claim 67, or a pharmaceutically acceptable salt thereof, wherein R_{4A} is —H or C₁-C₆ alkyl.

70. The compound of claim 1, selected from the following:

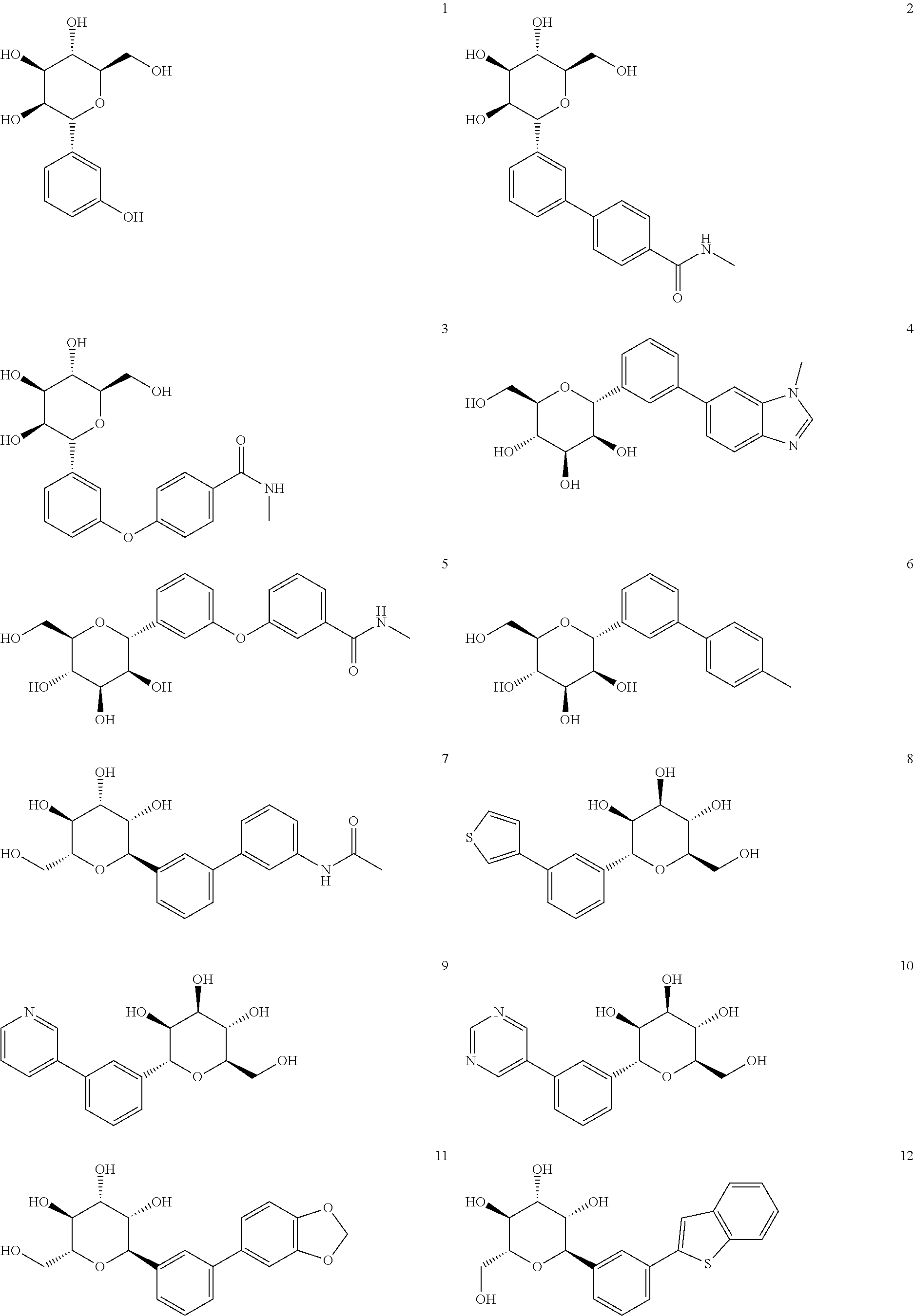

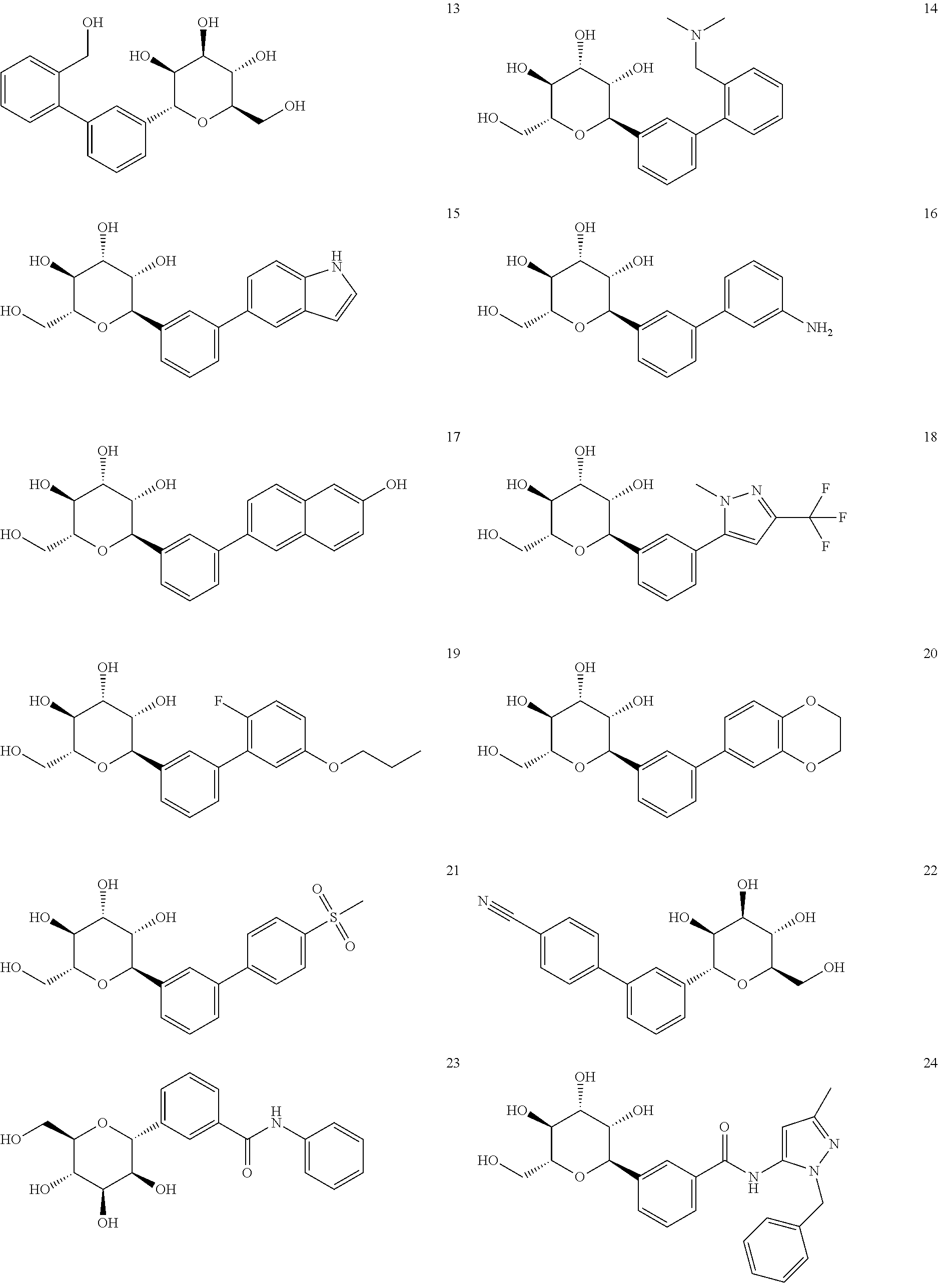

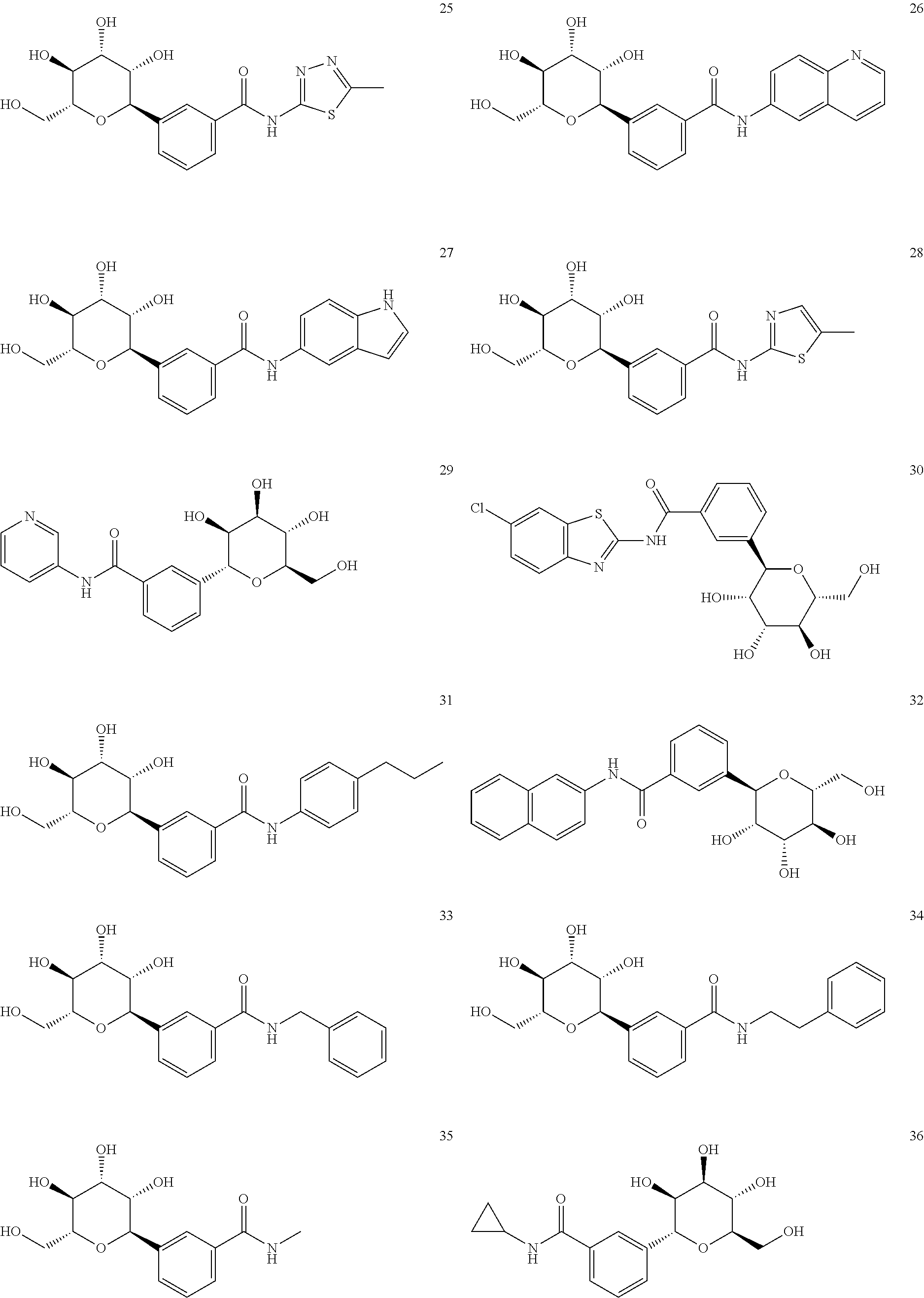

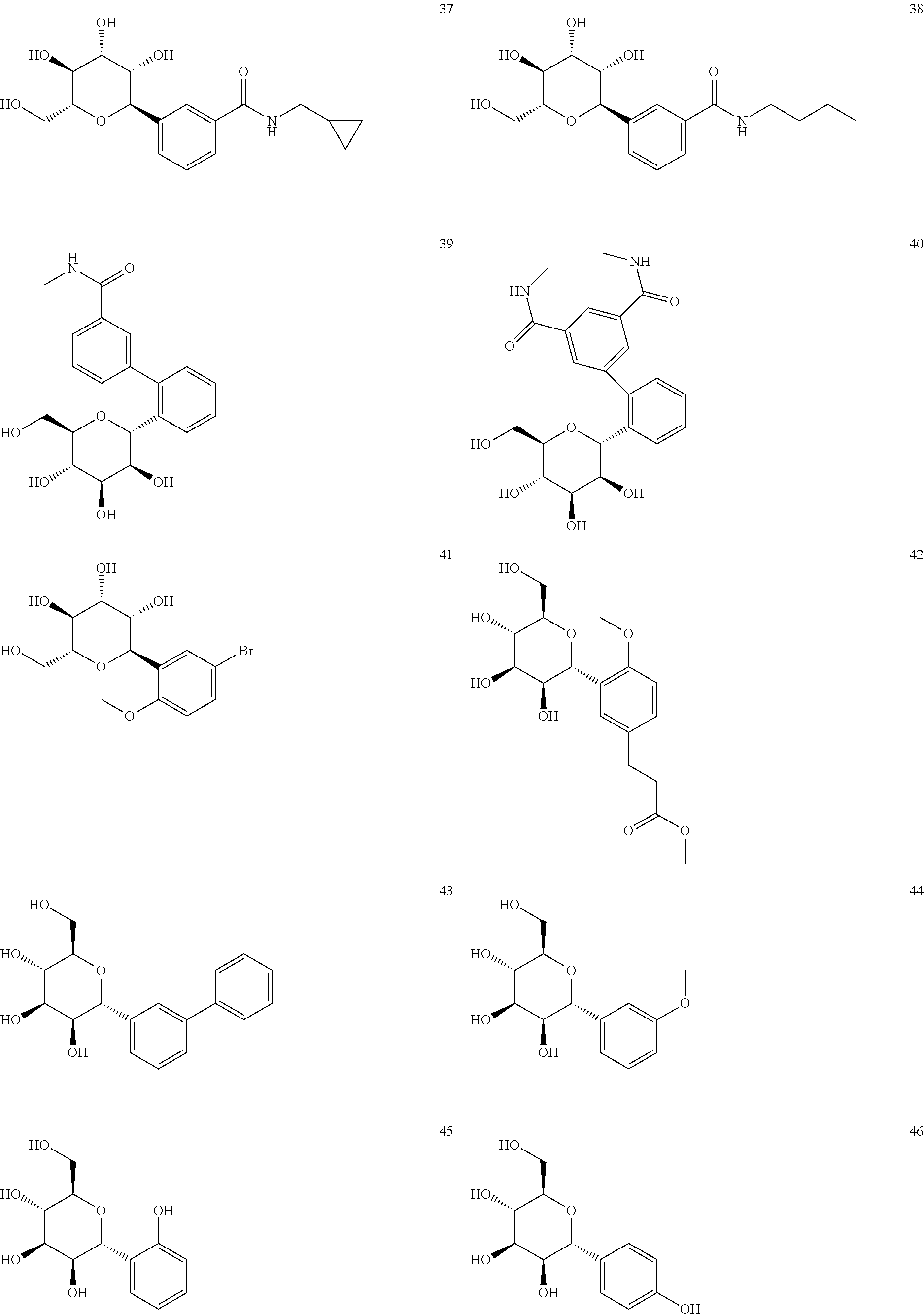

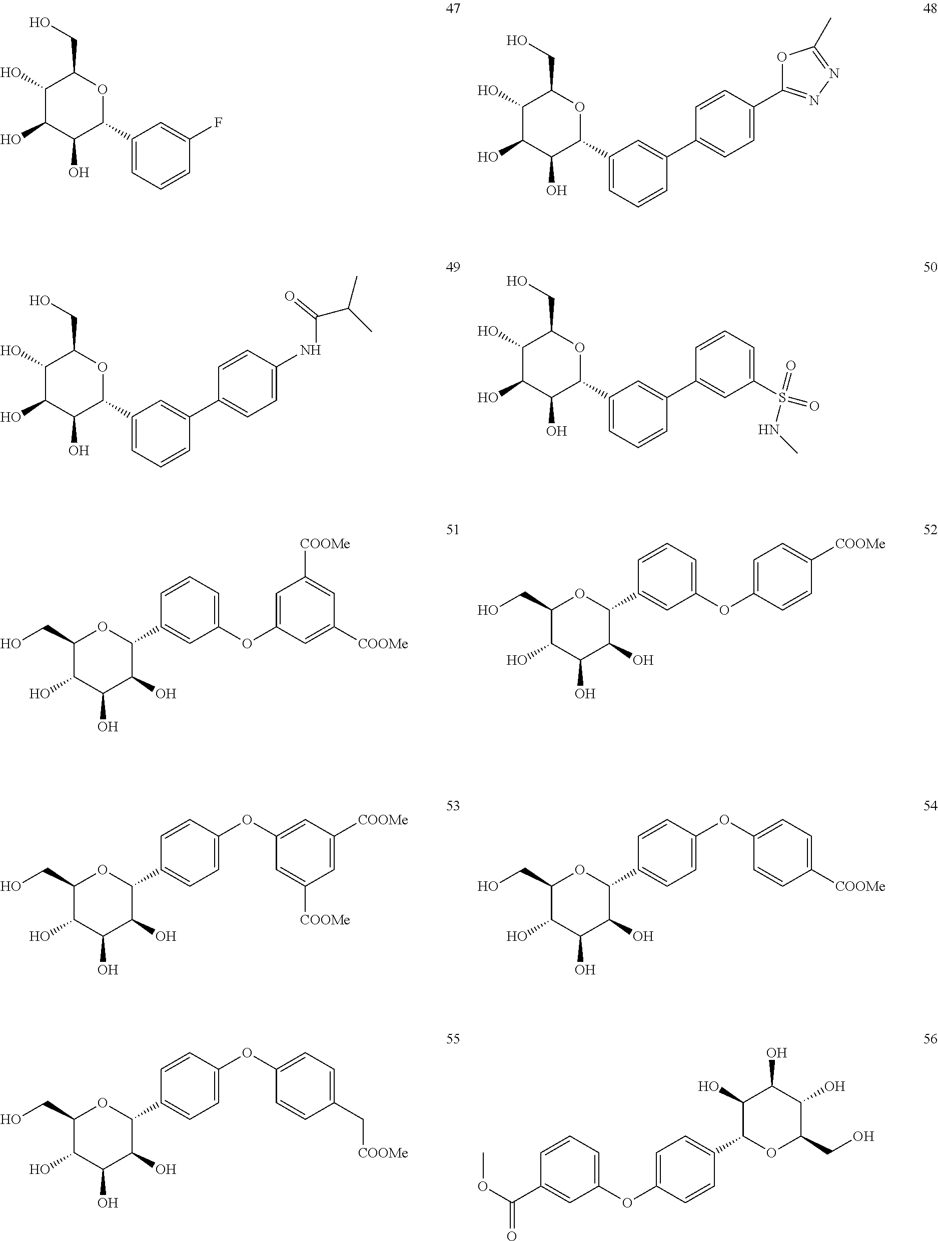

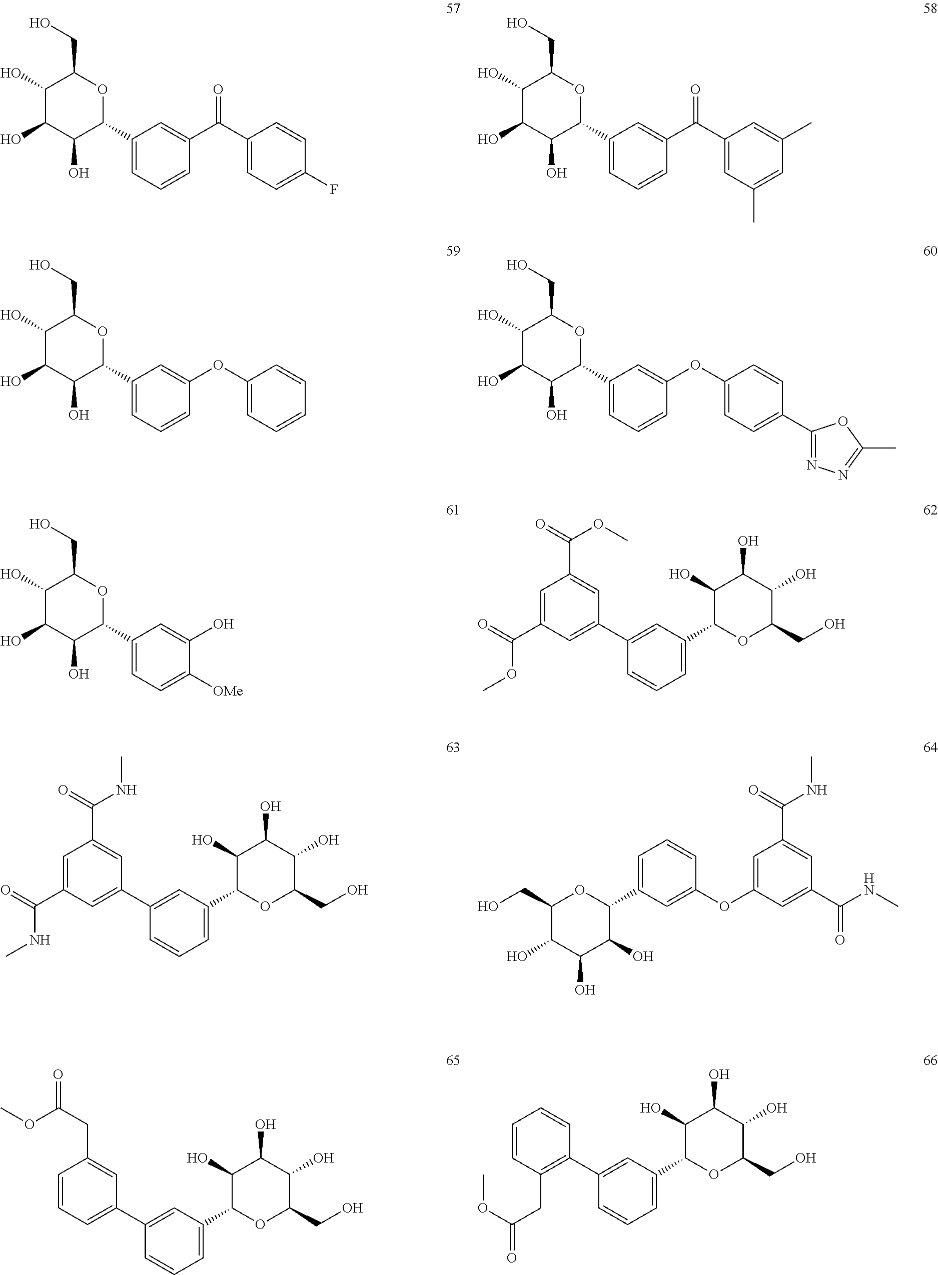

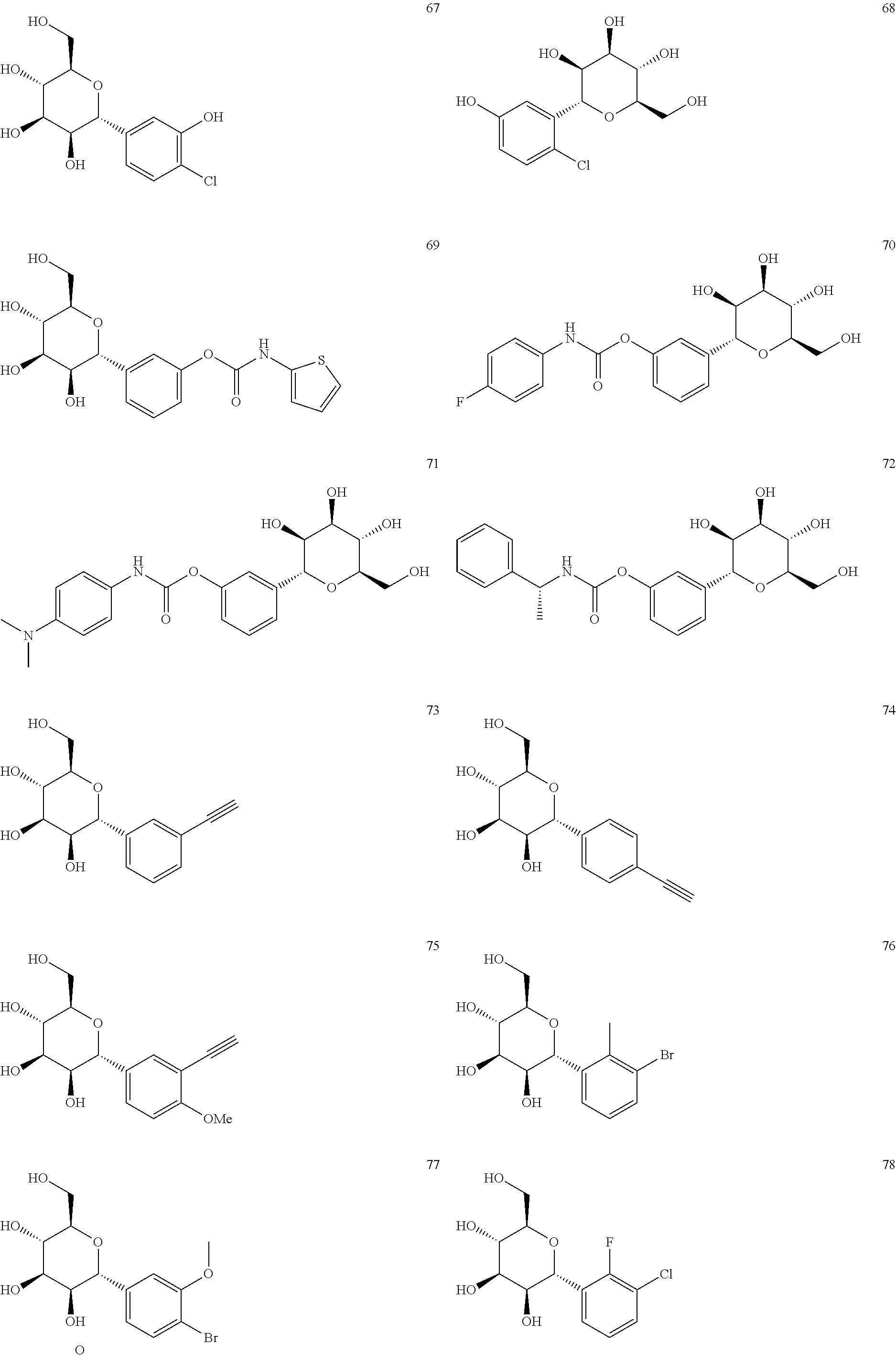

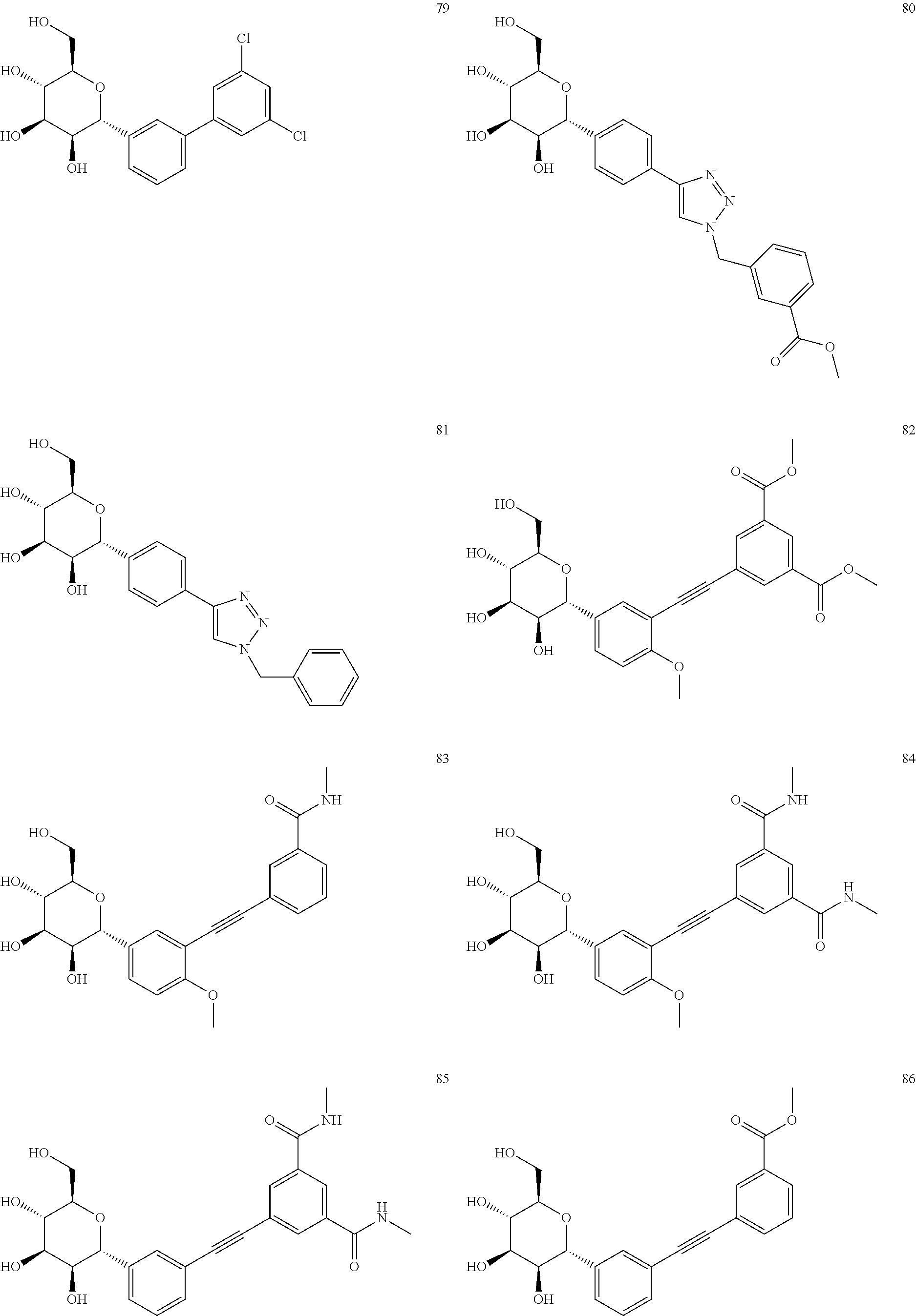

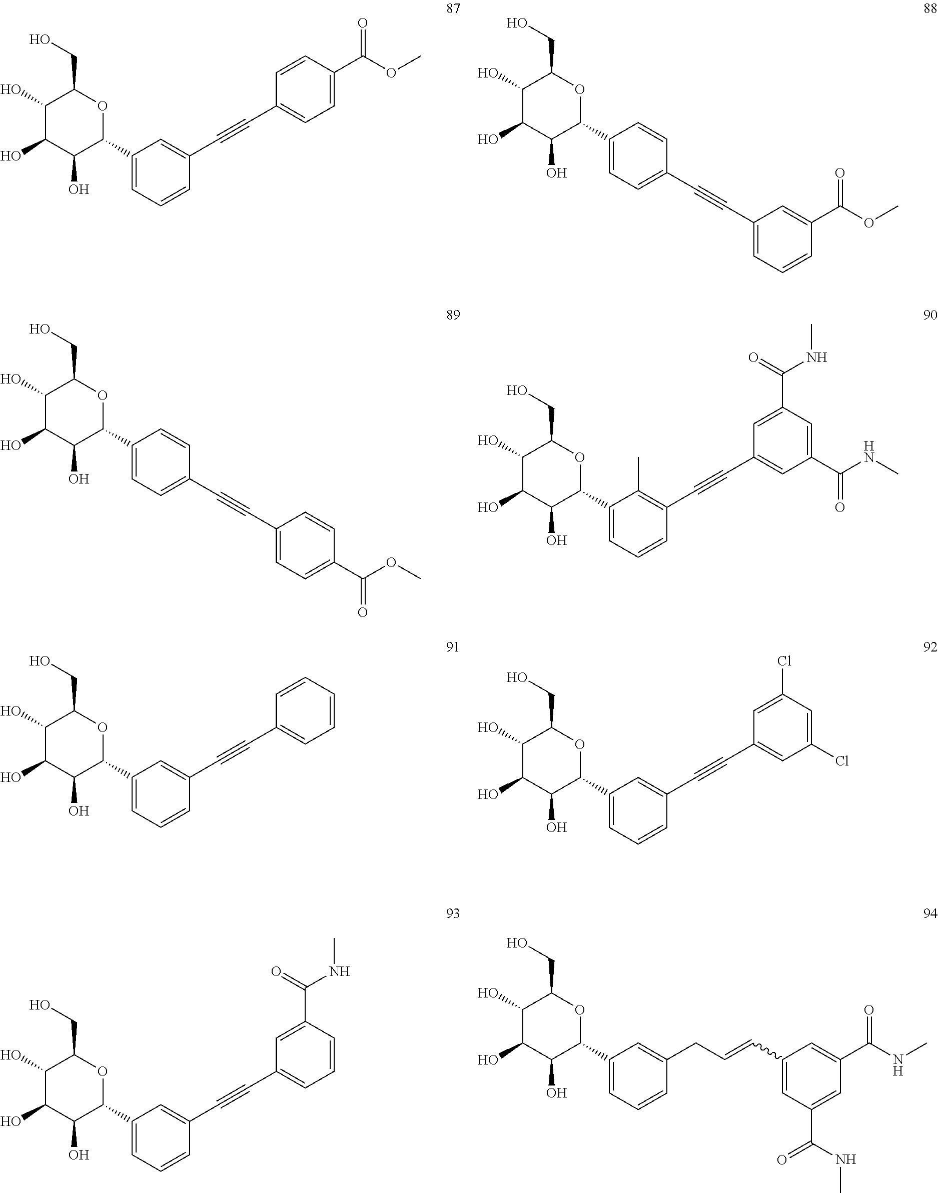

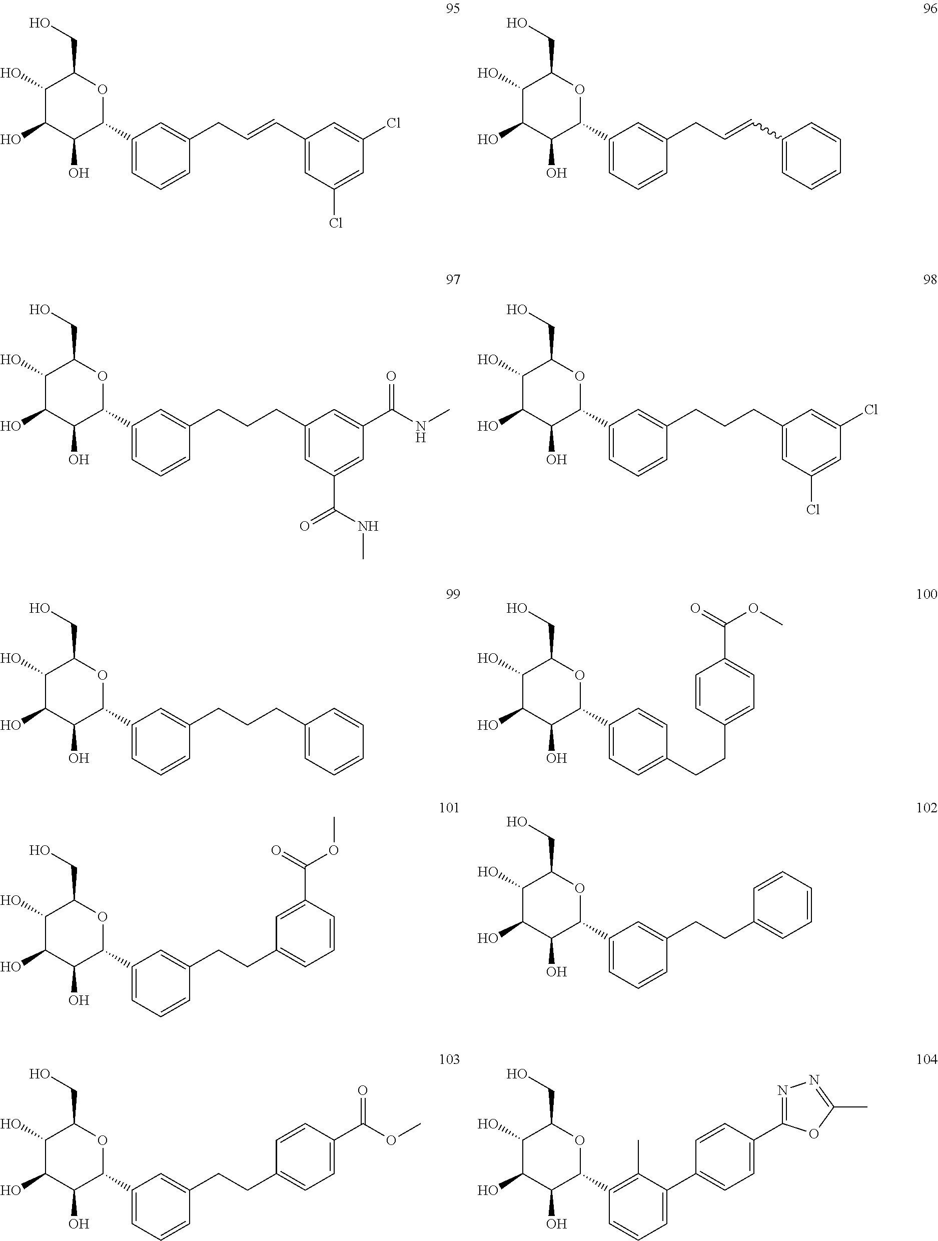

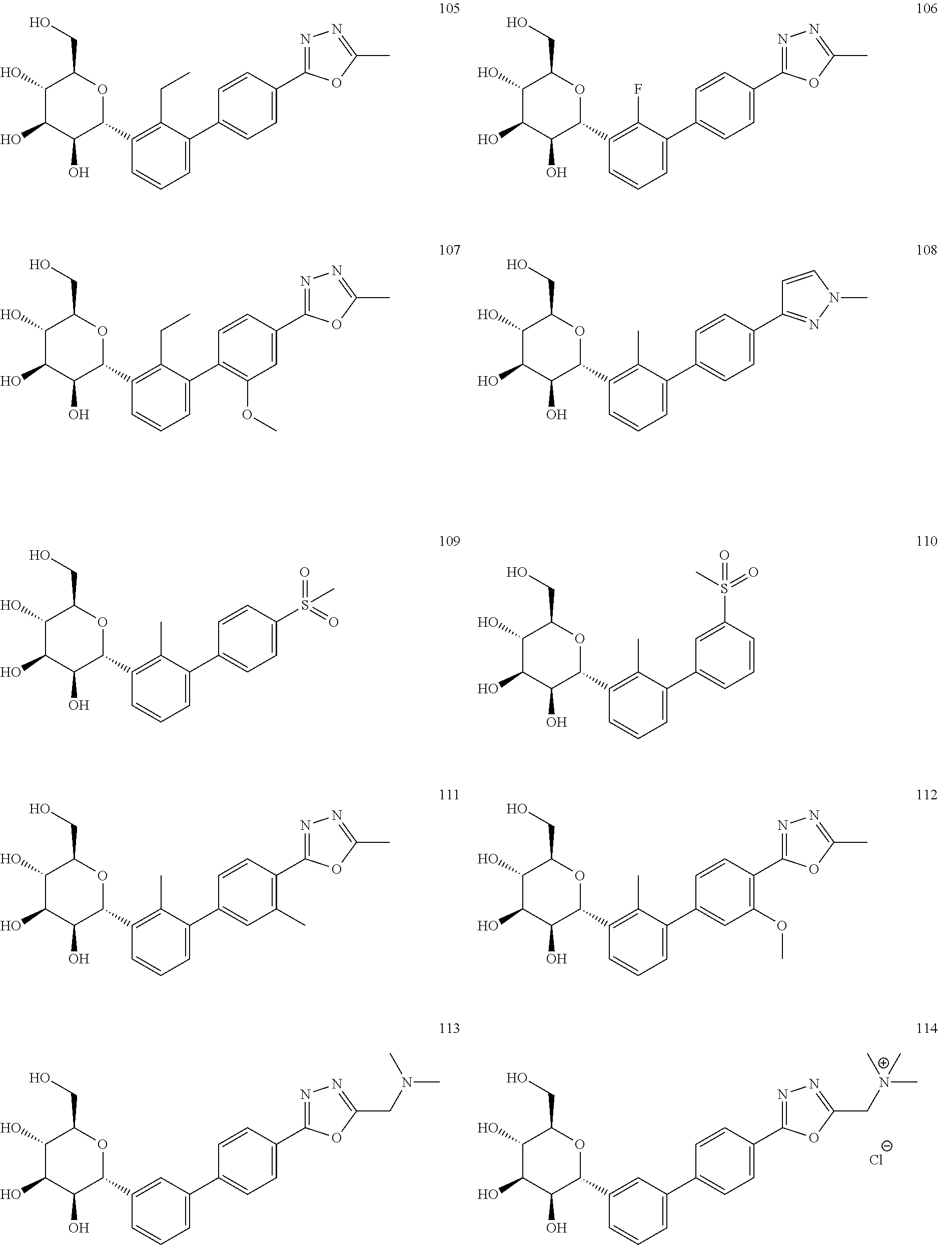

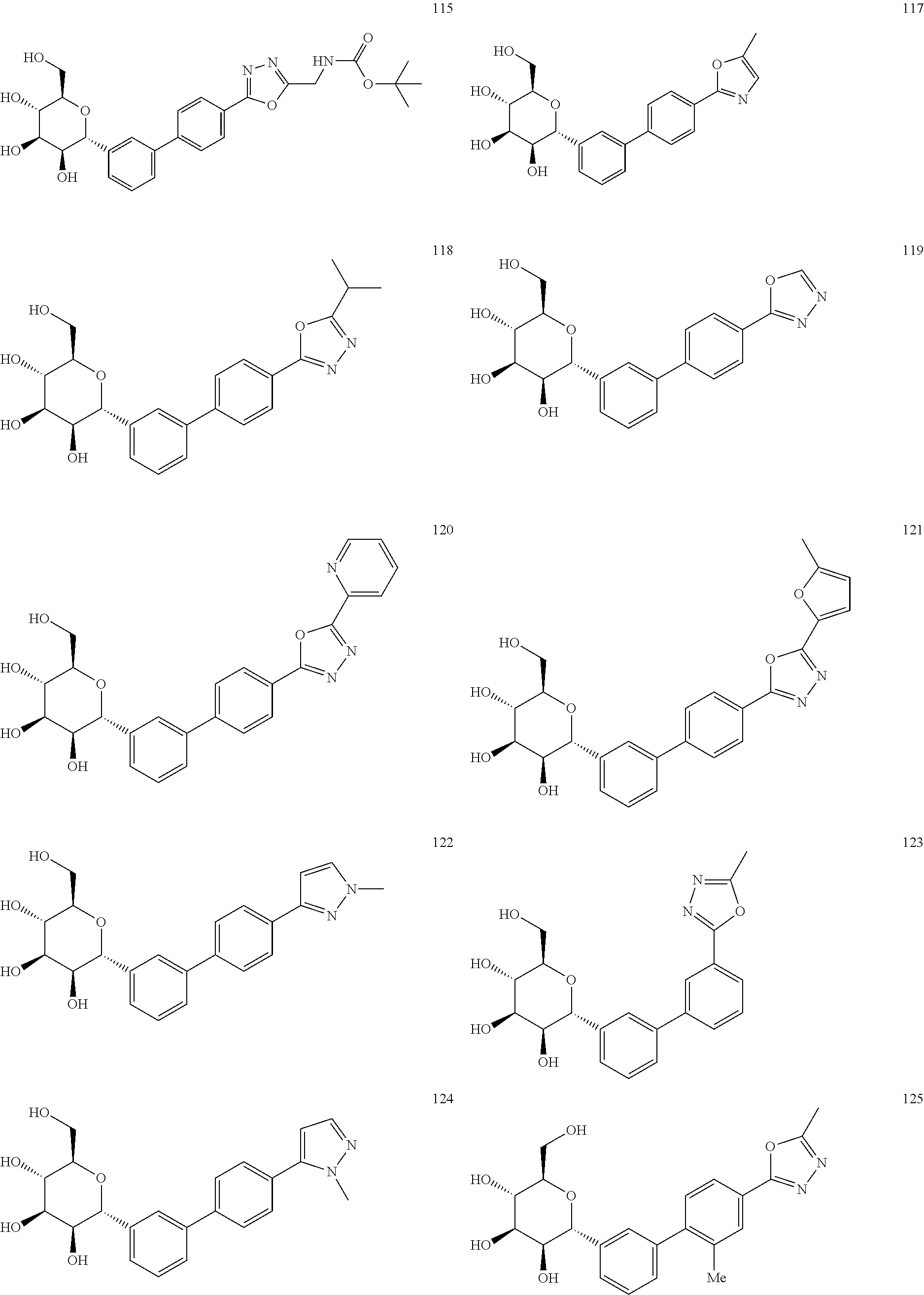

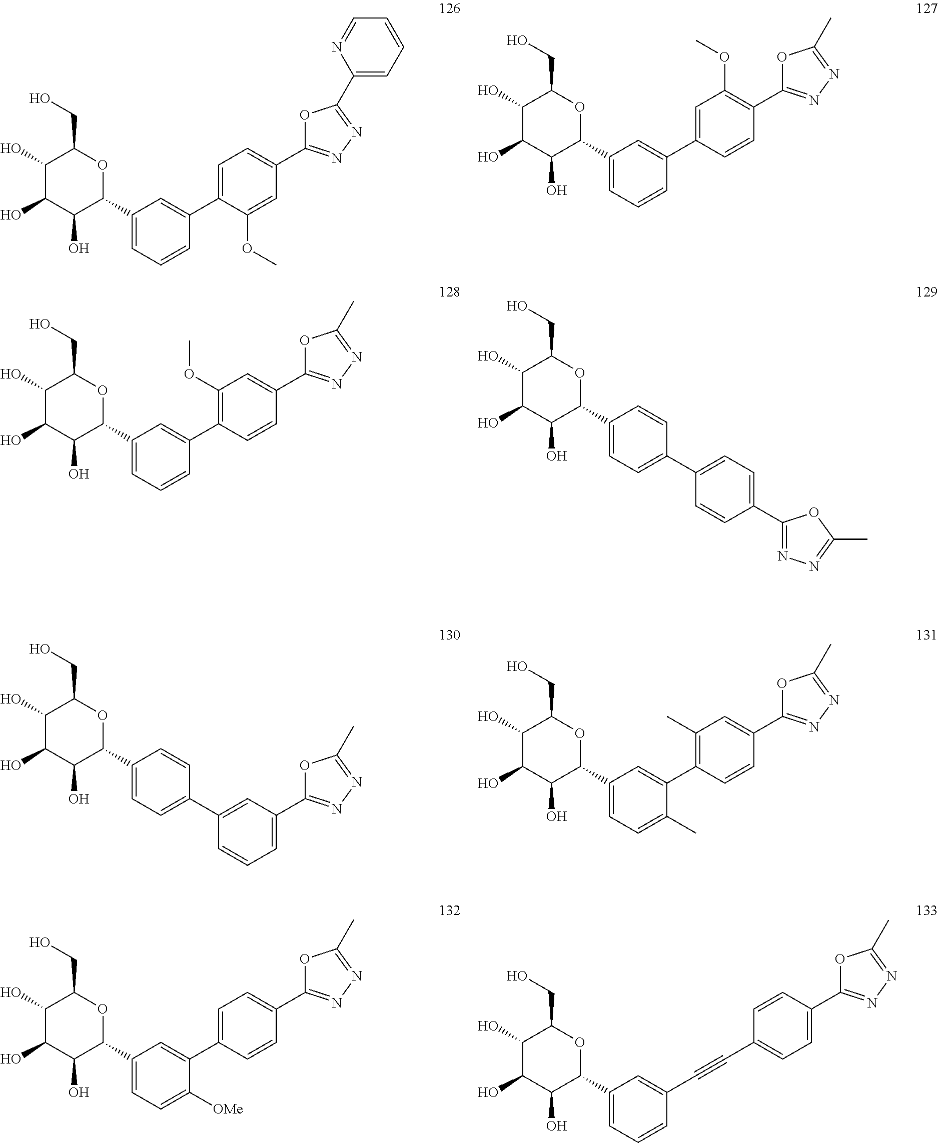

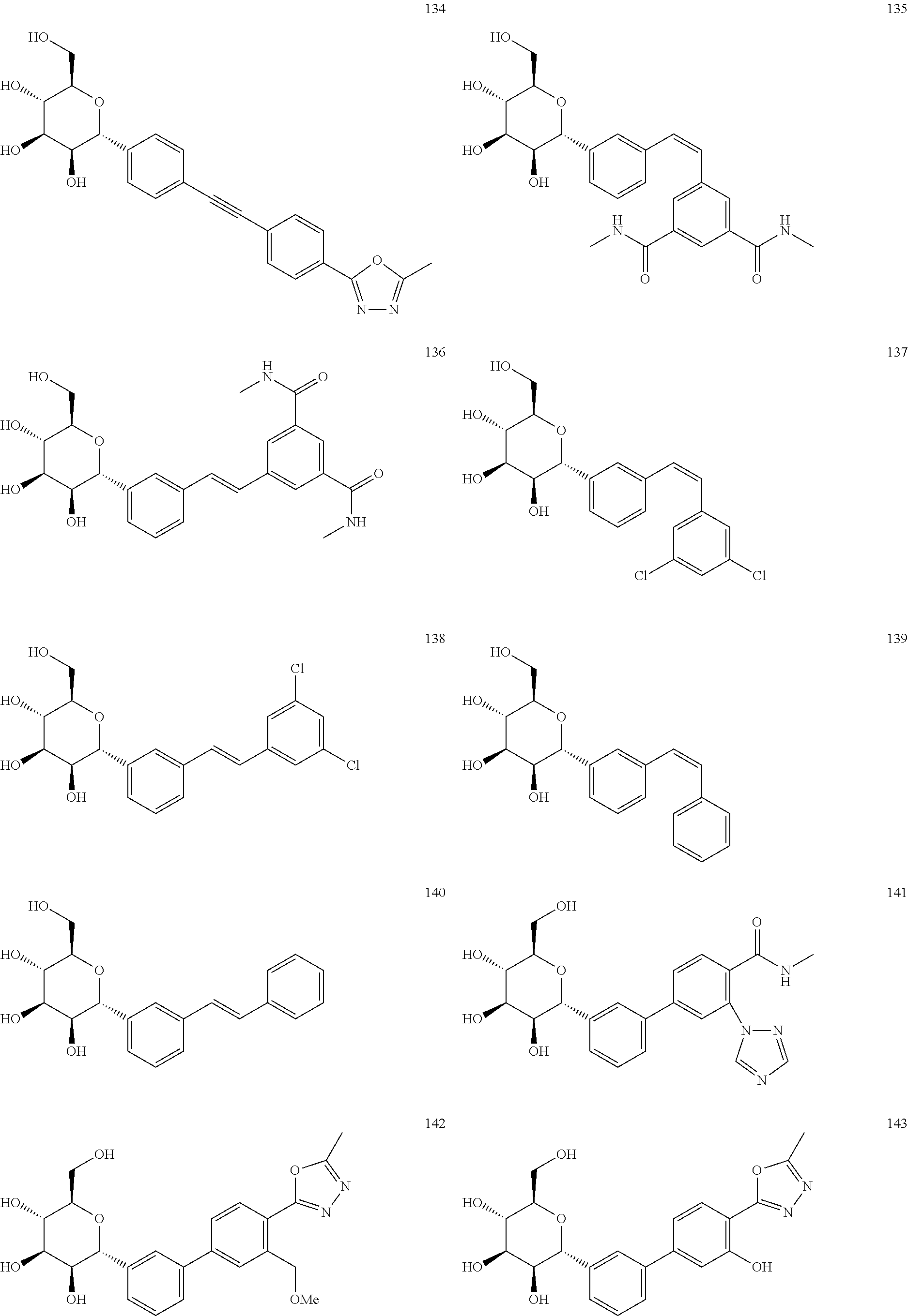

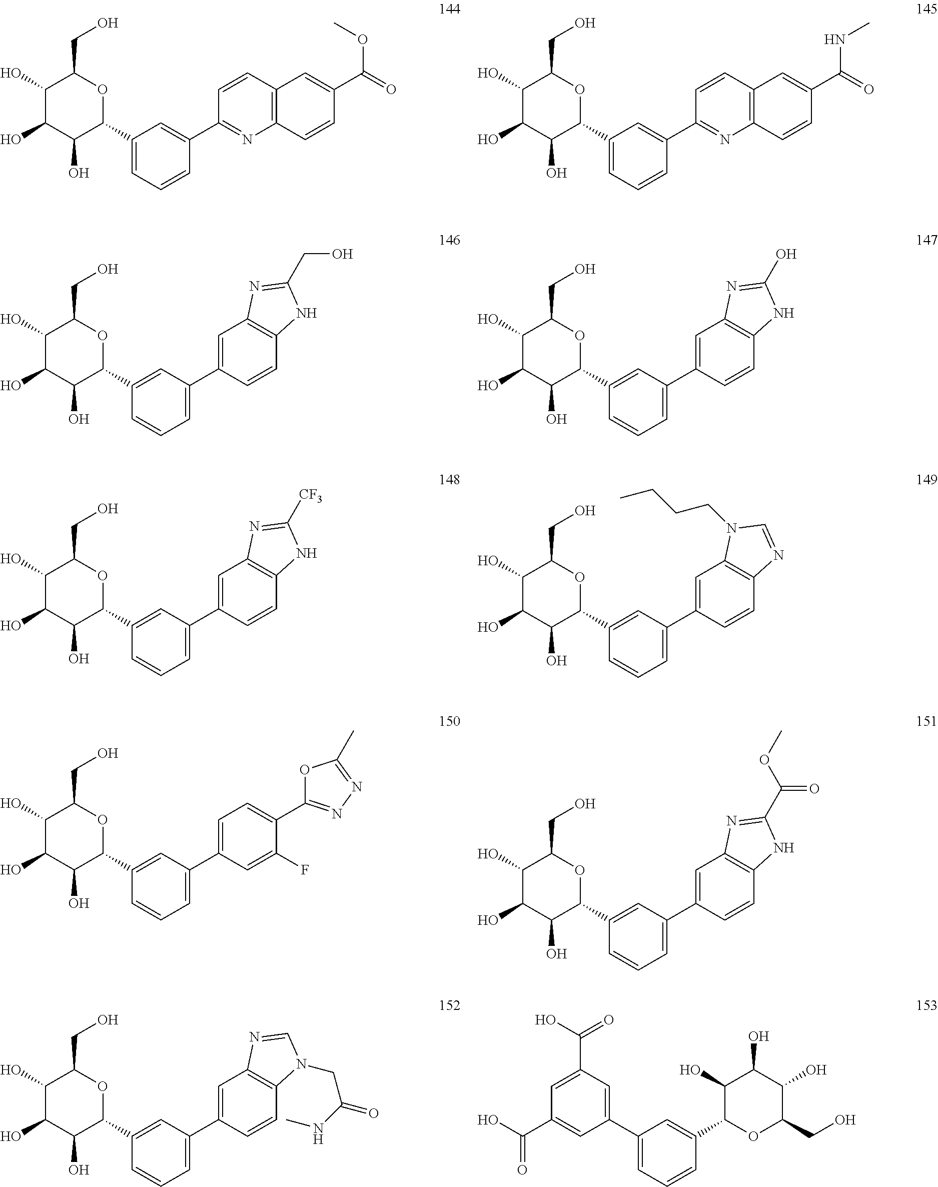

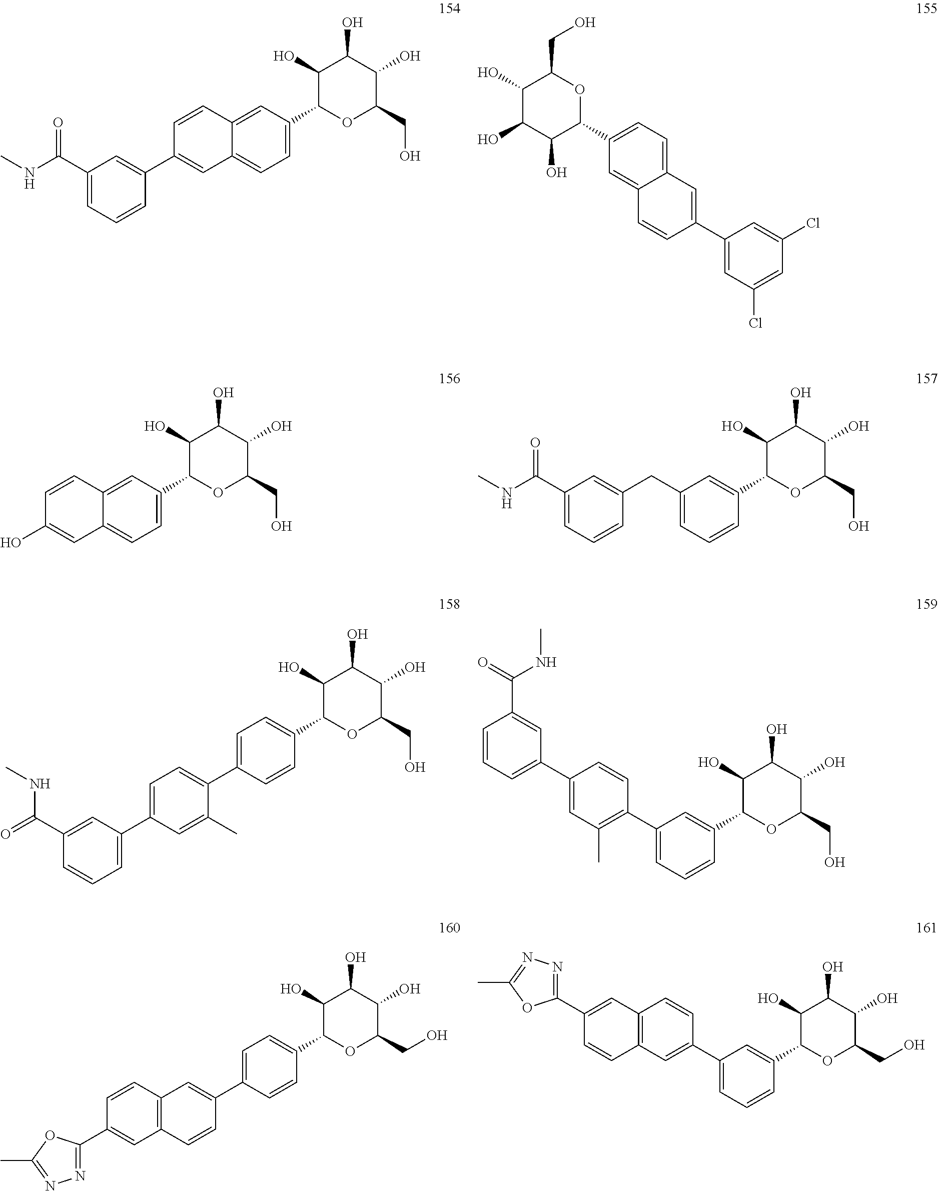

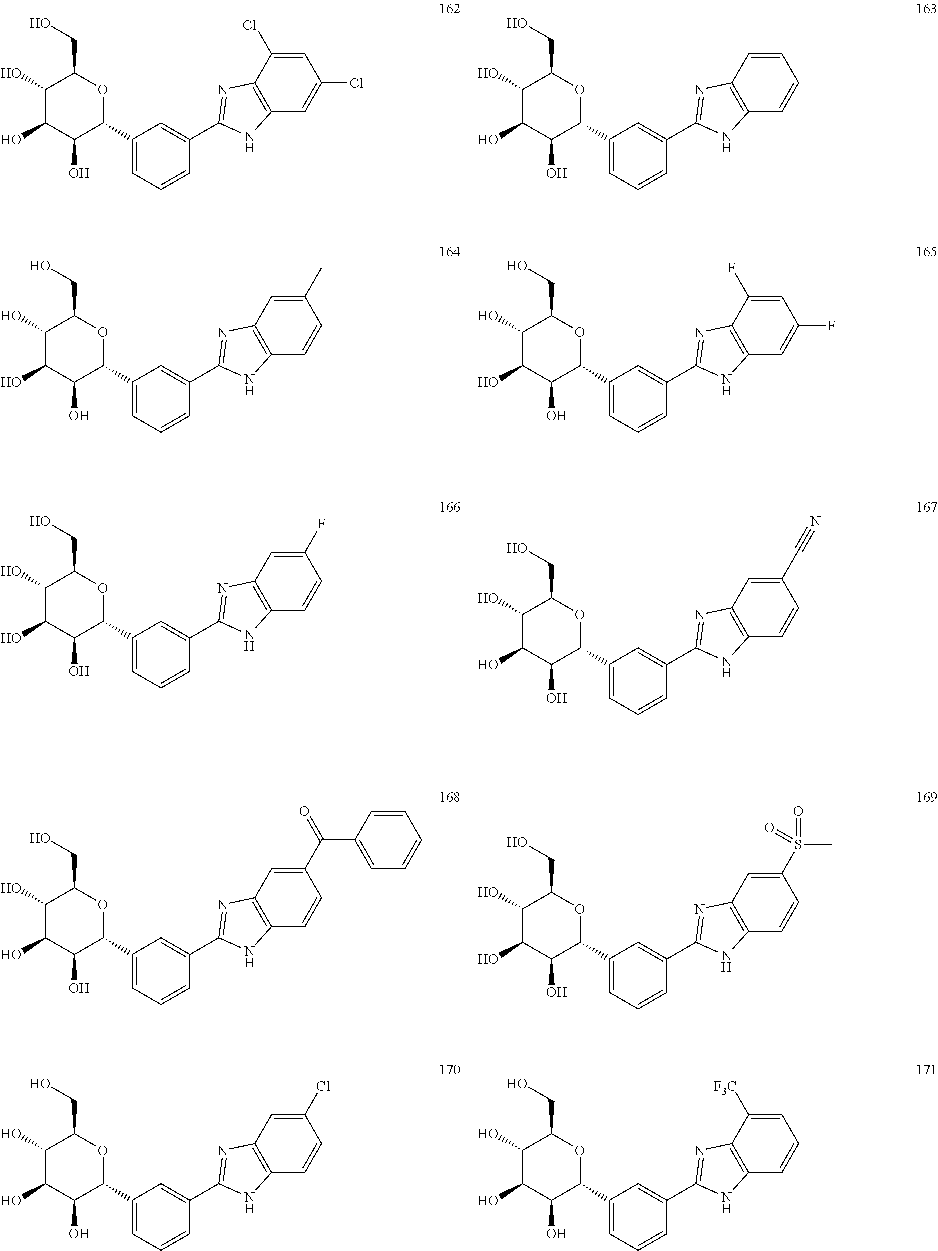

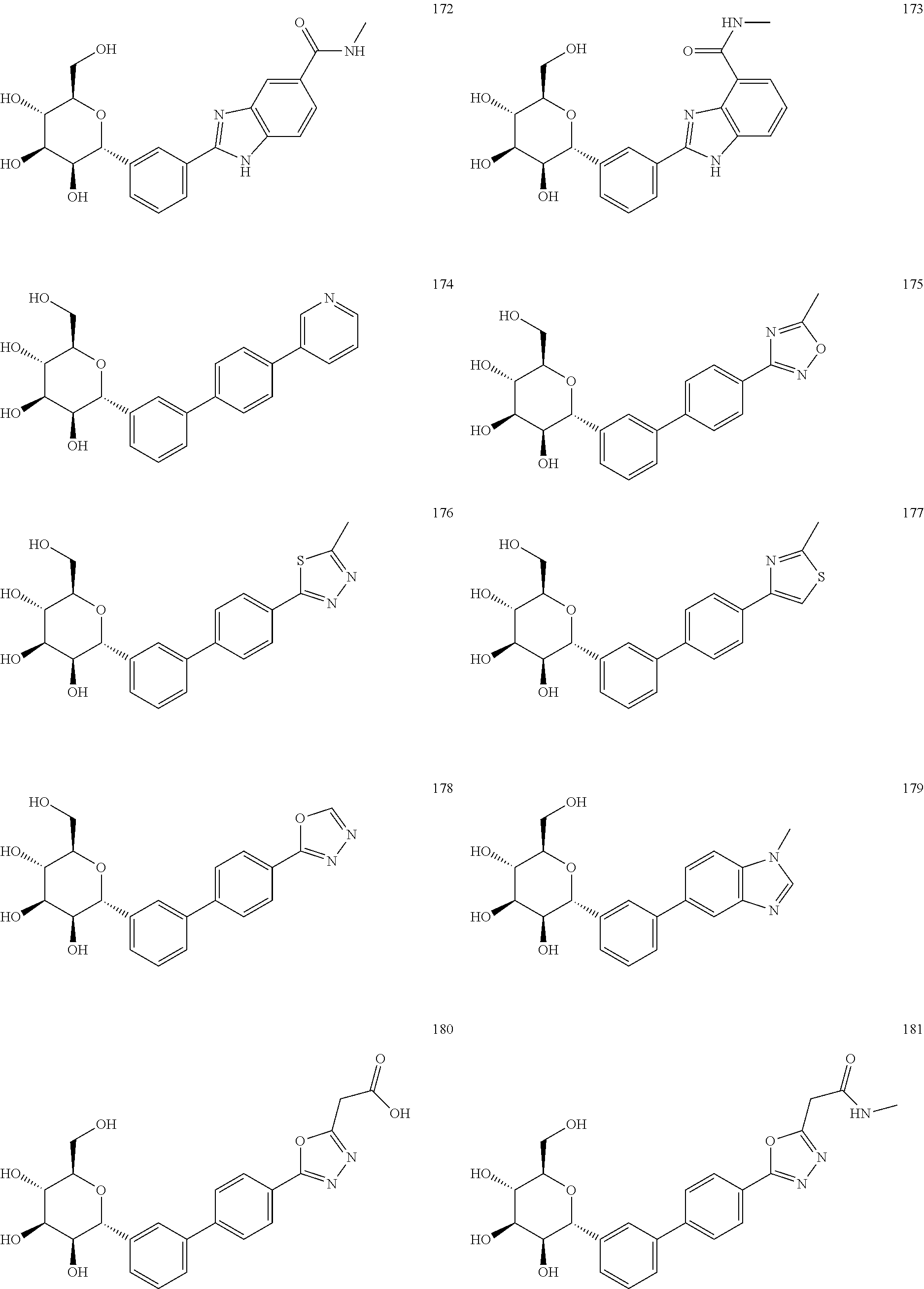

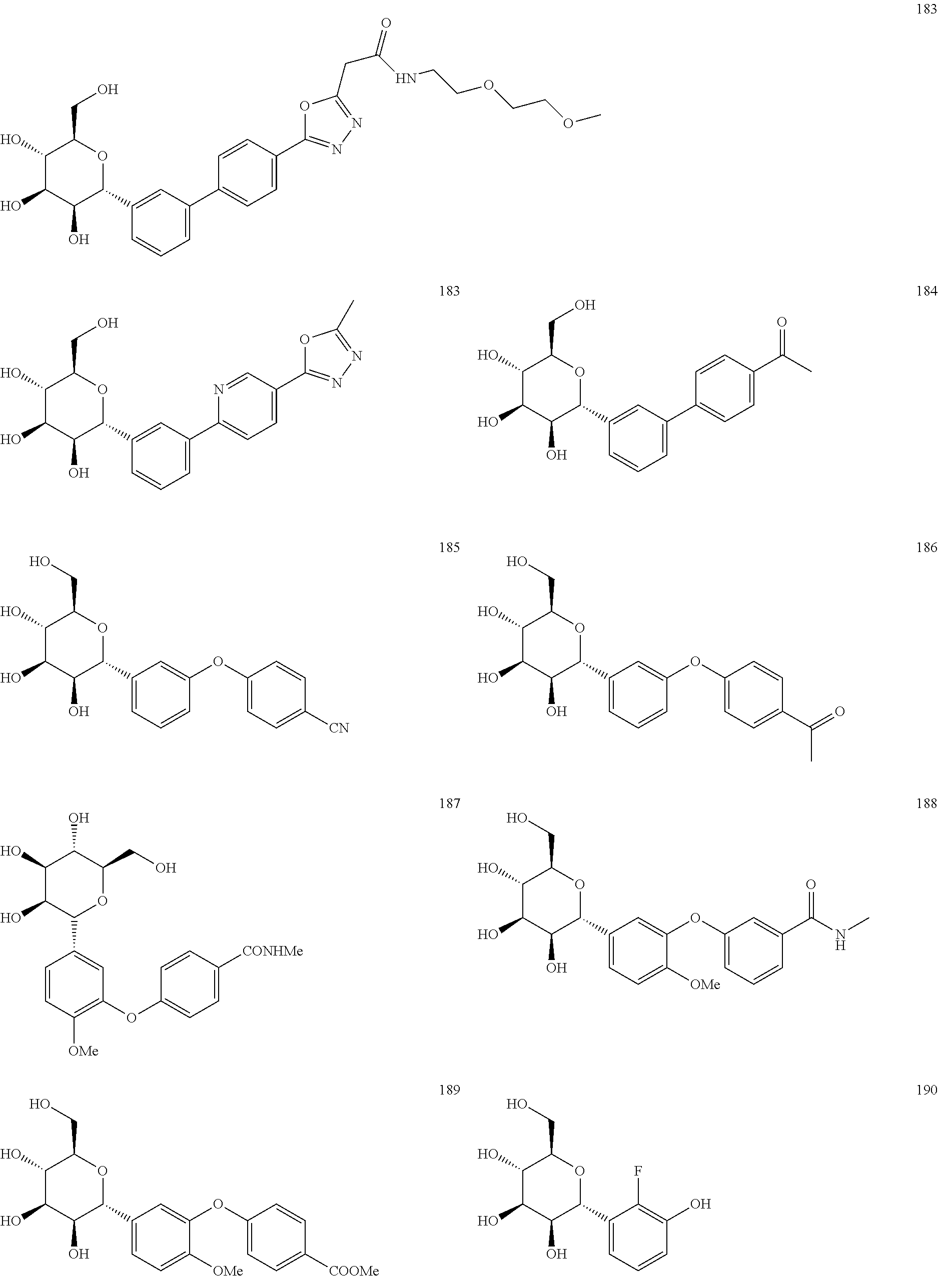

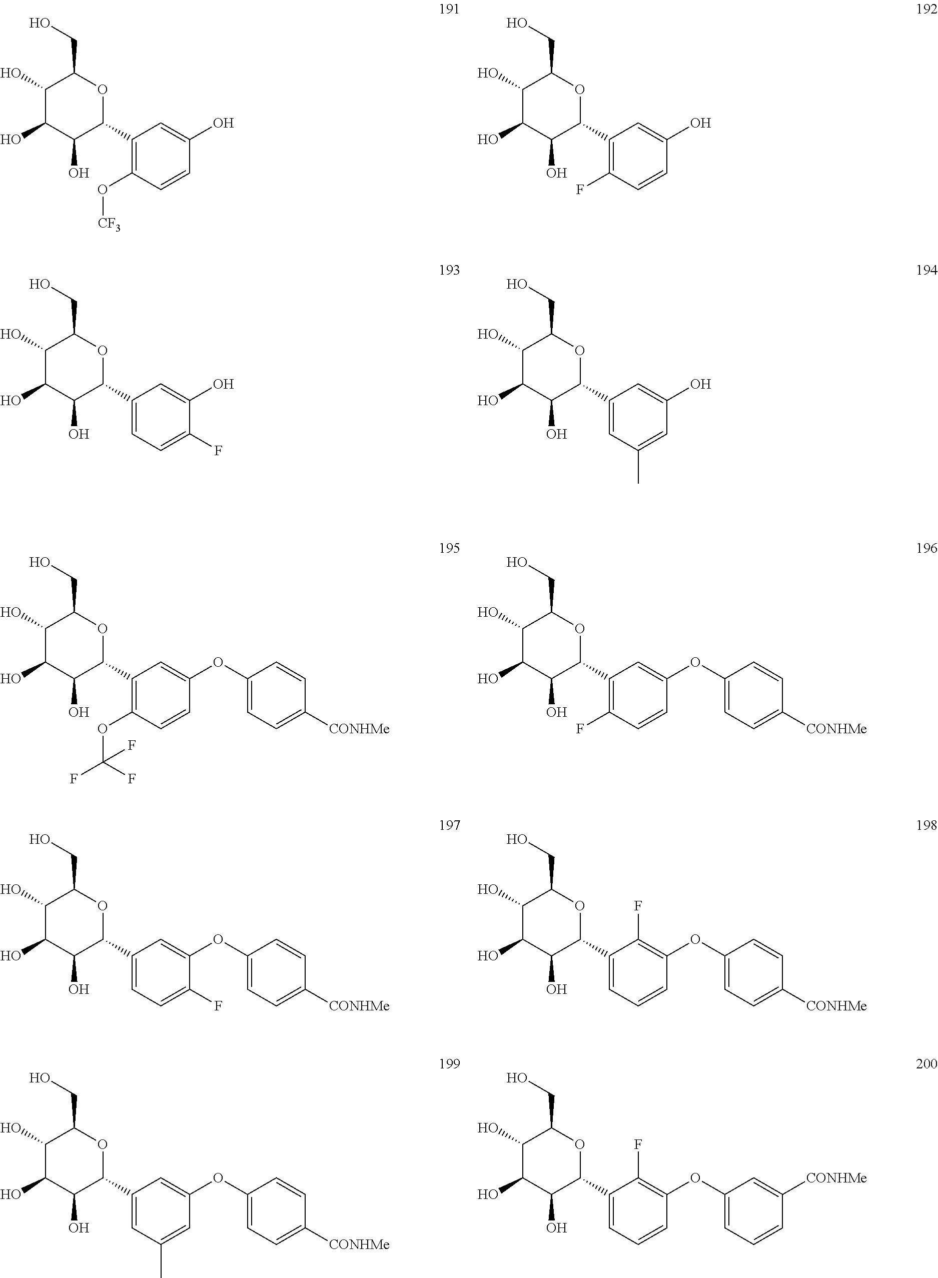

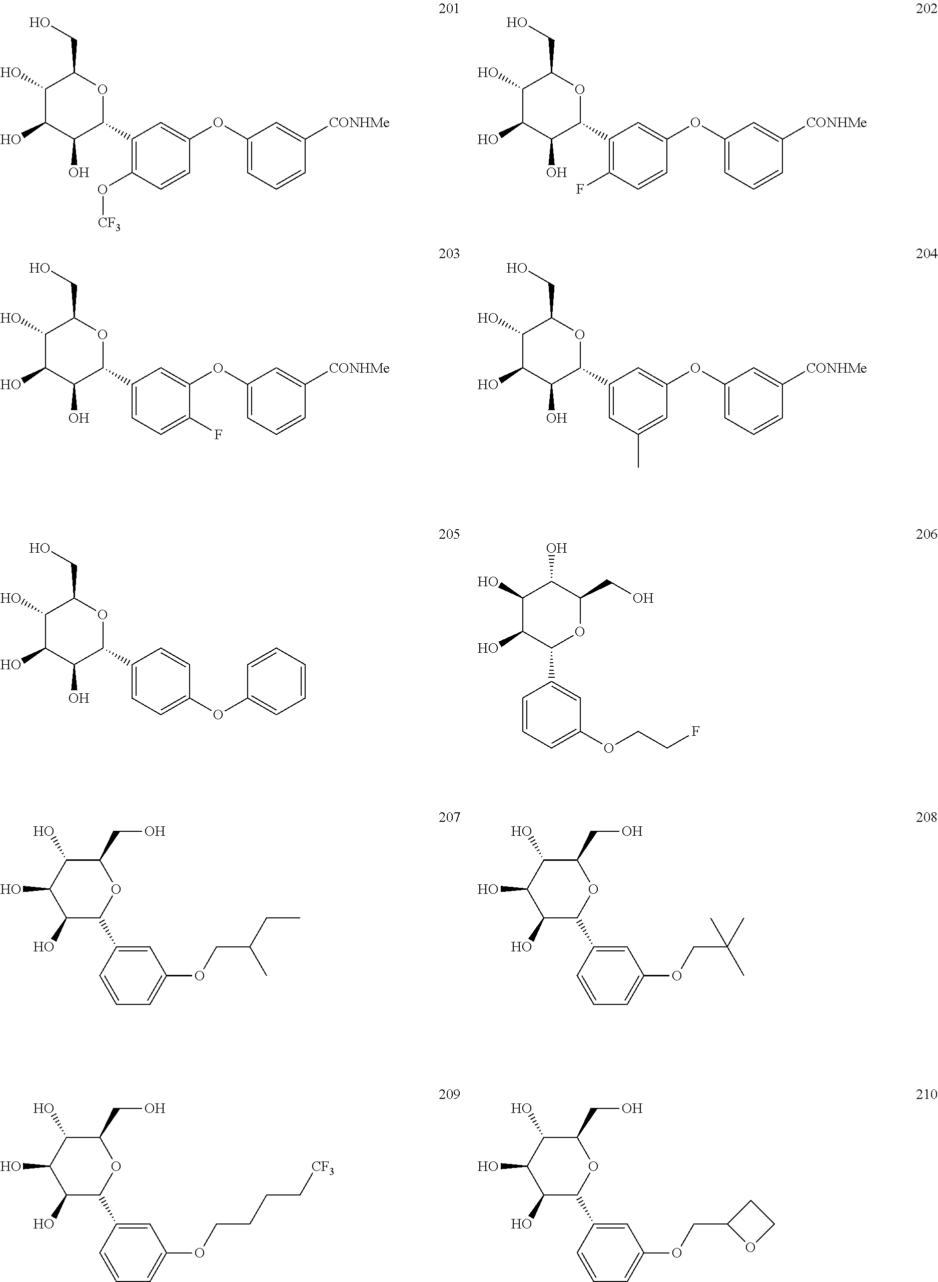

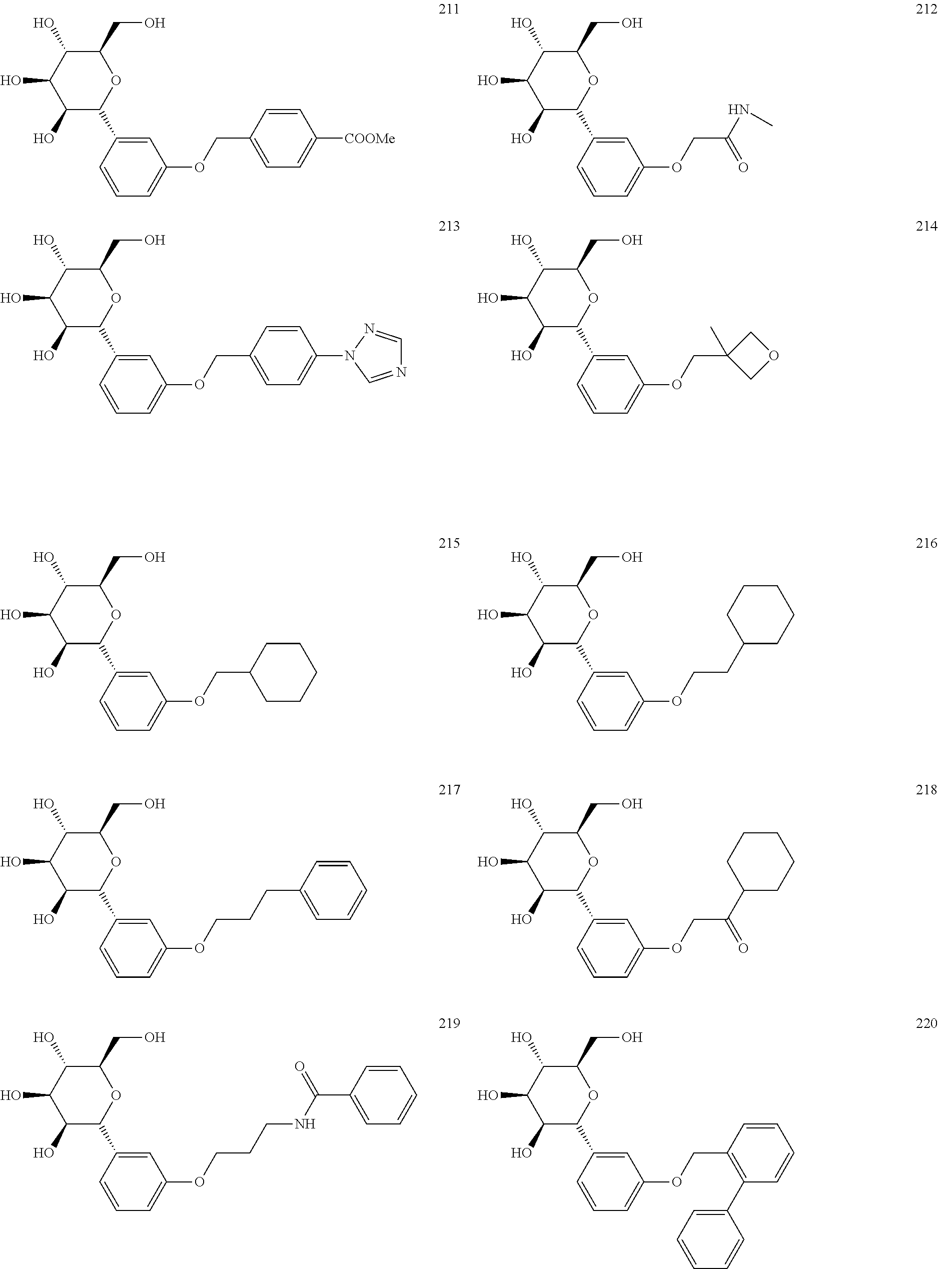

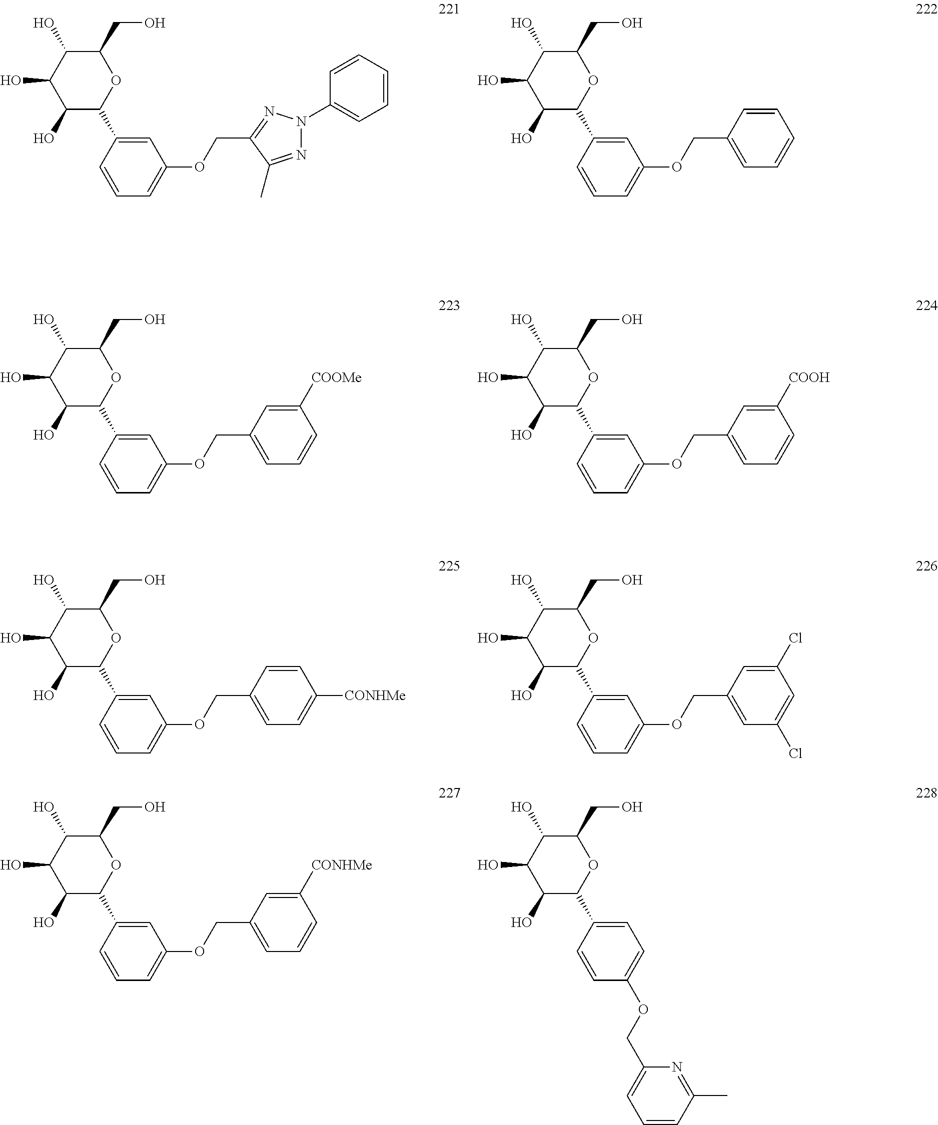

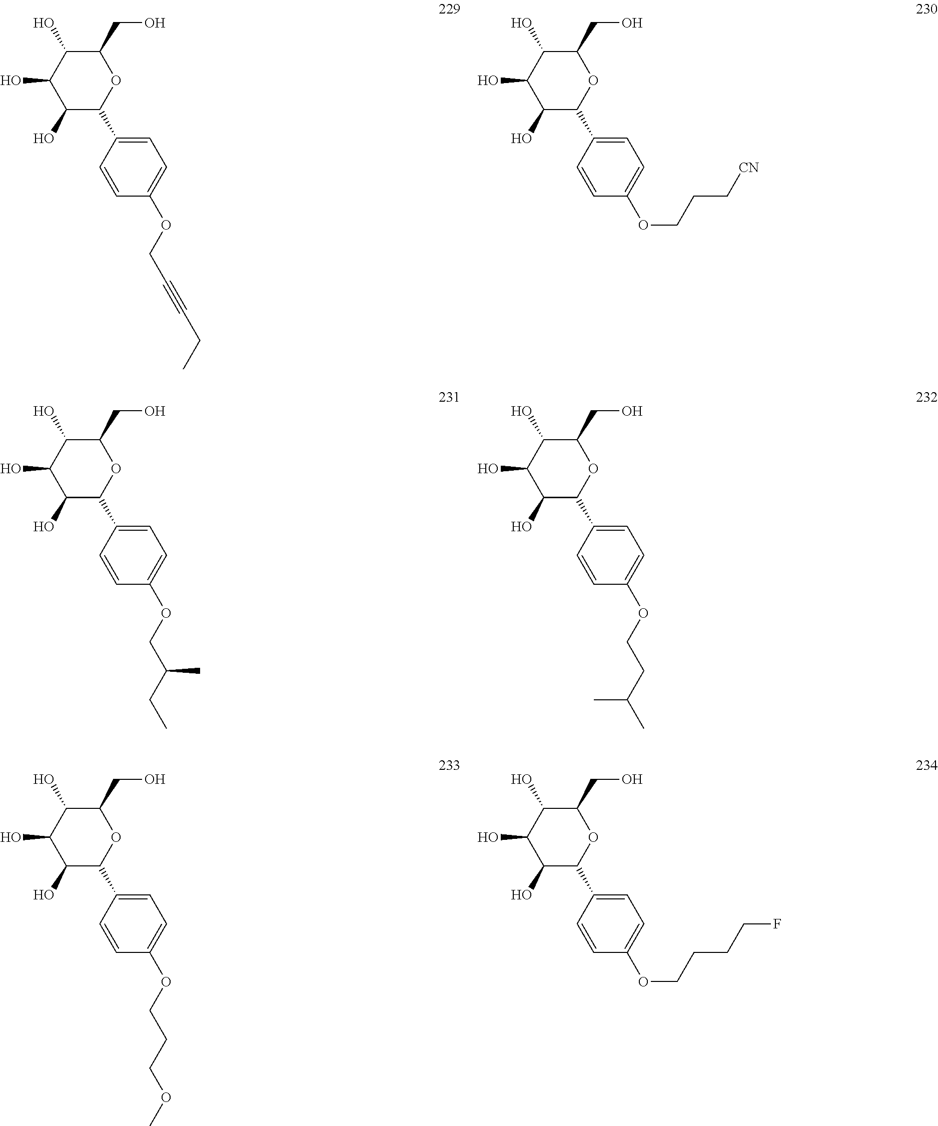

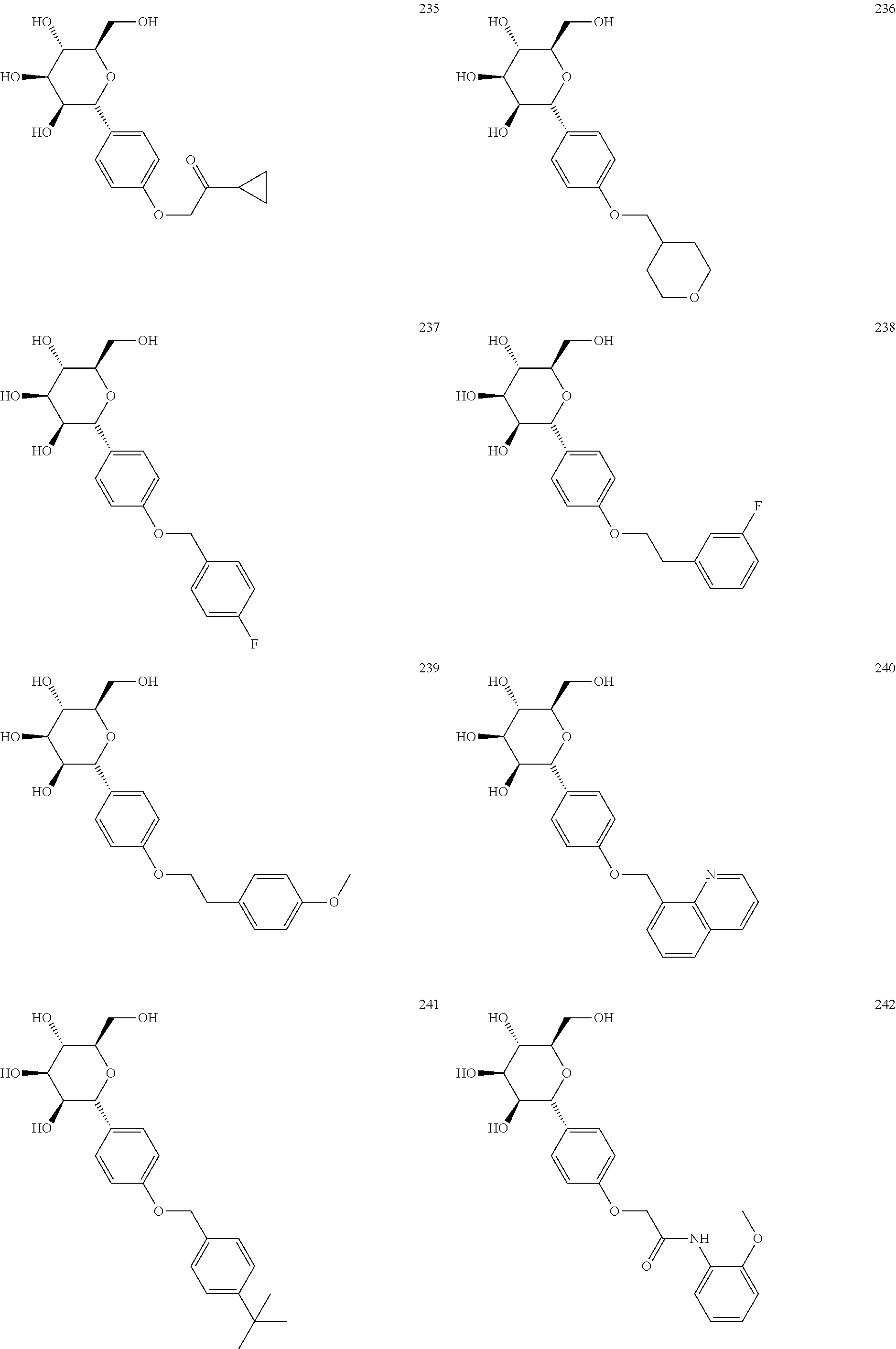

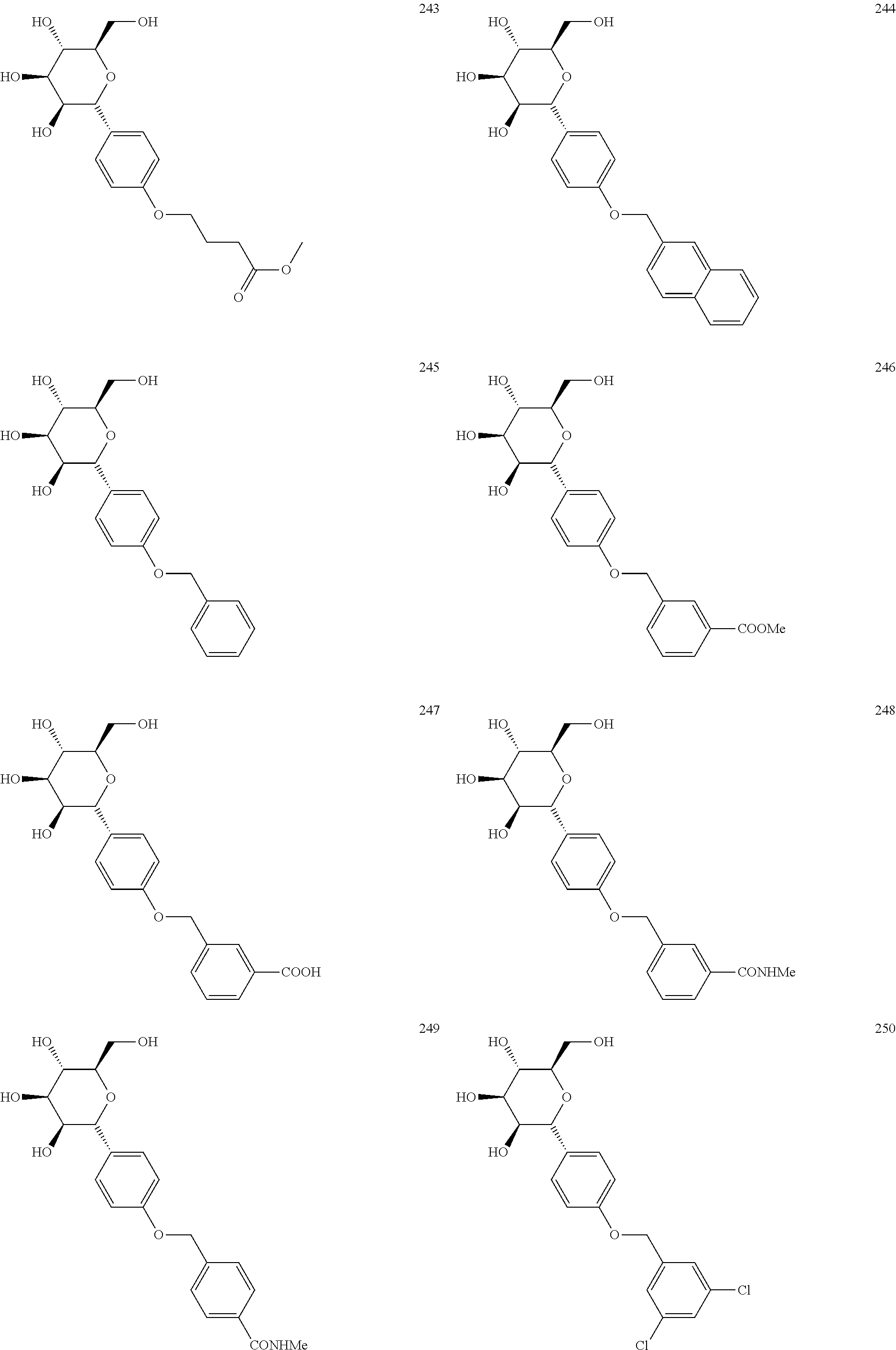

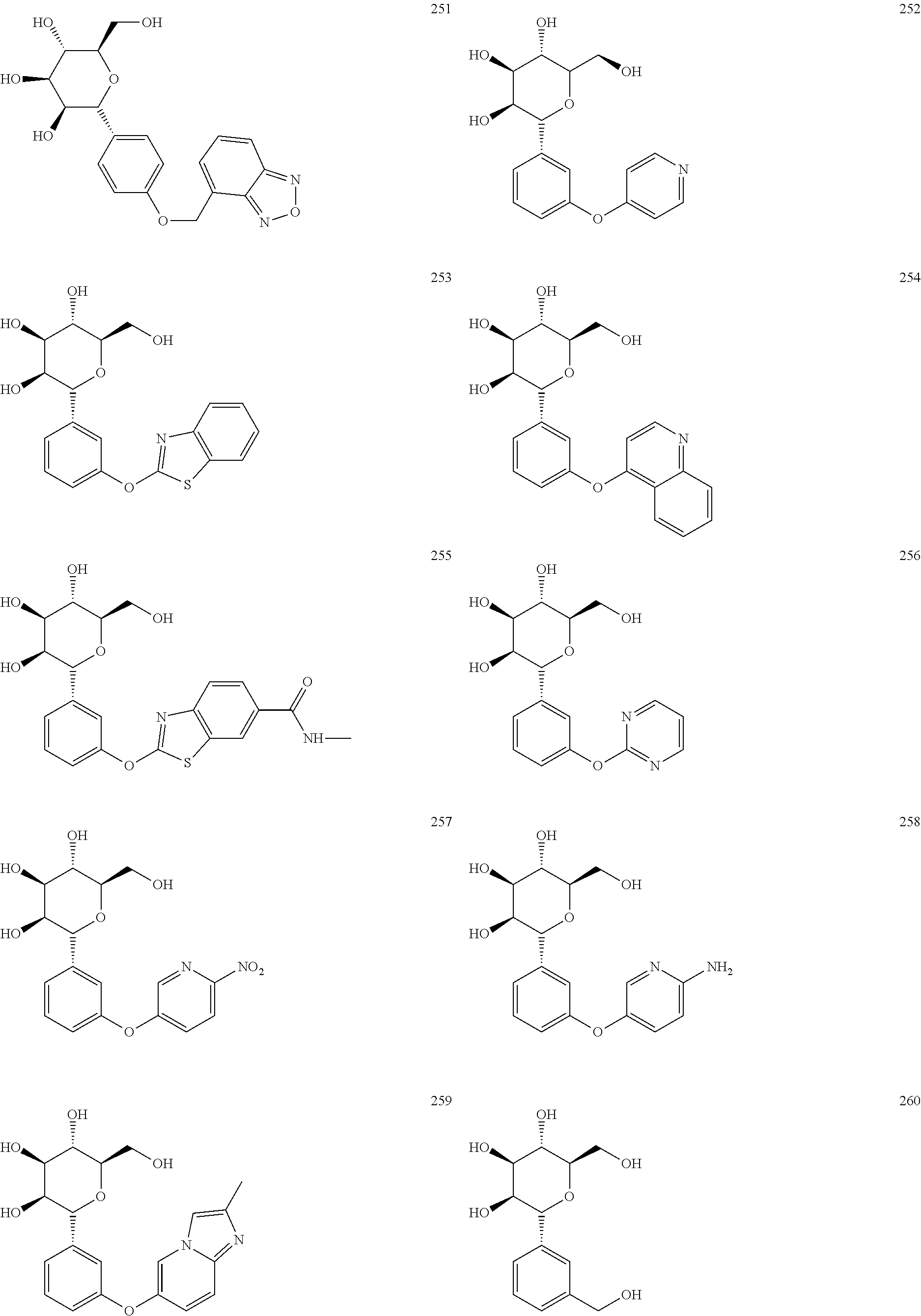

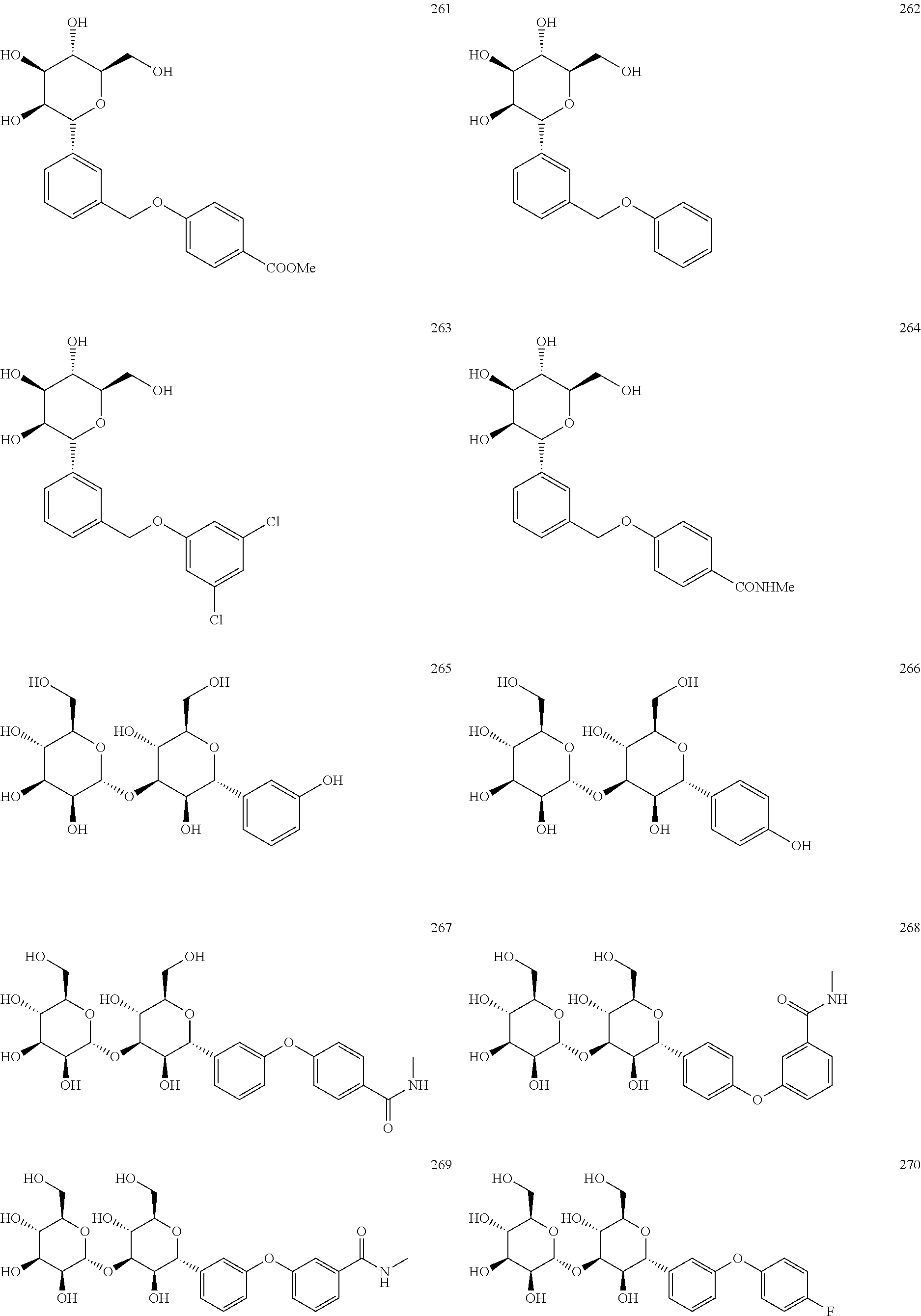

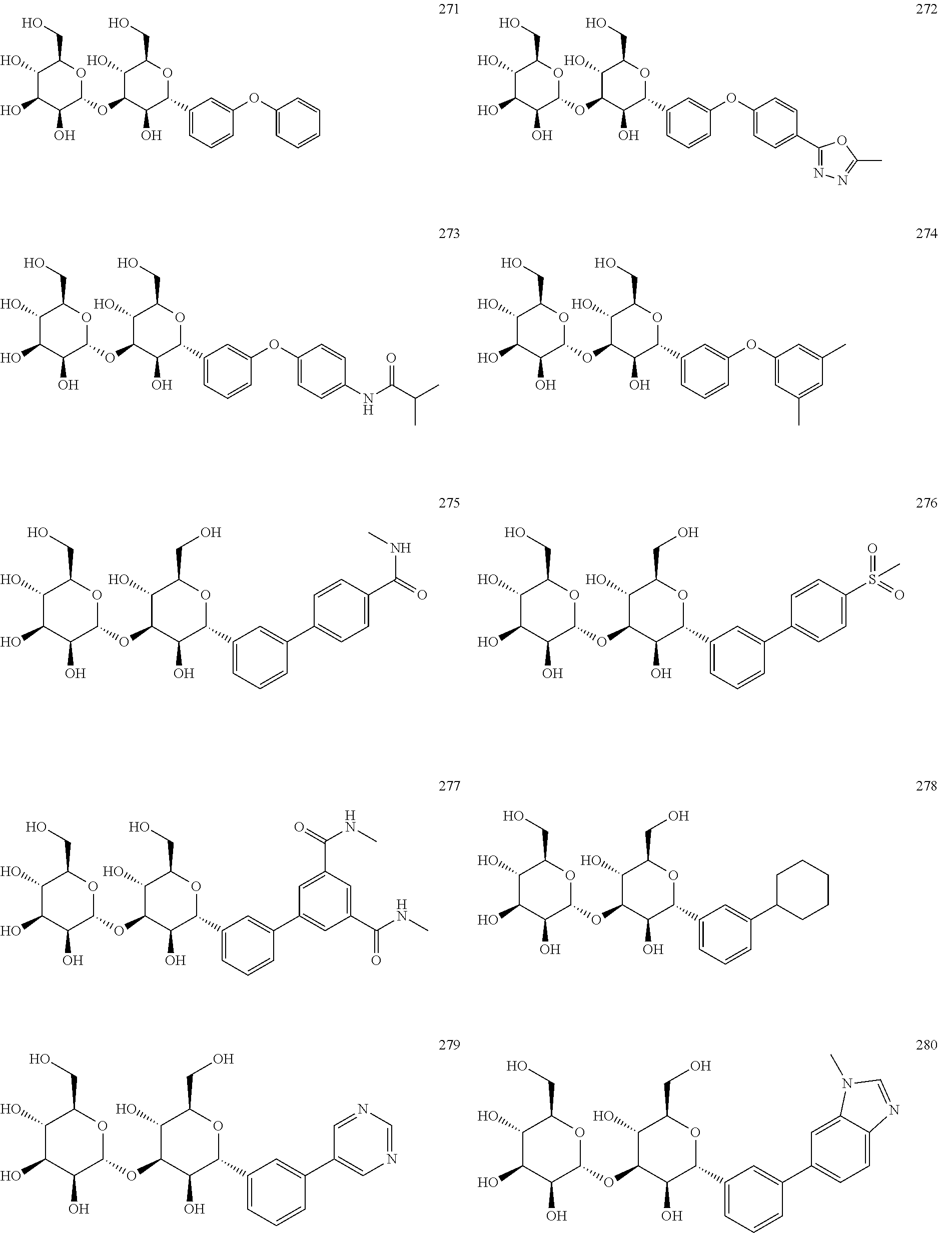

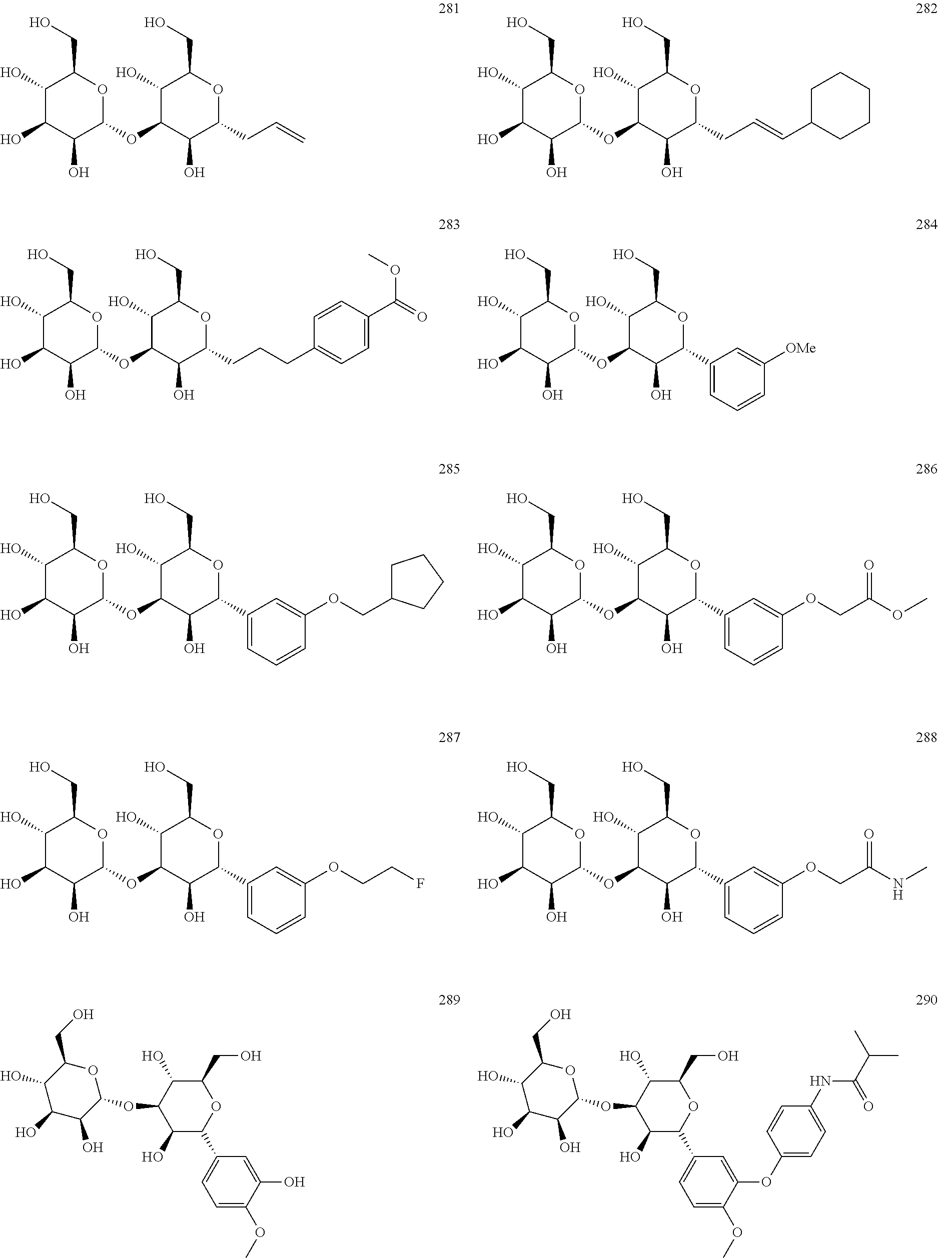

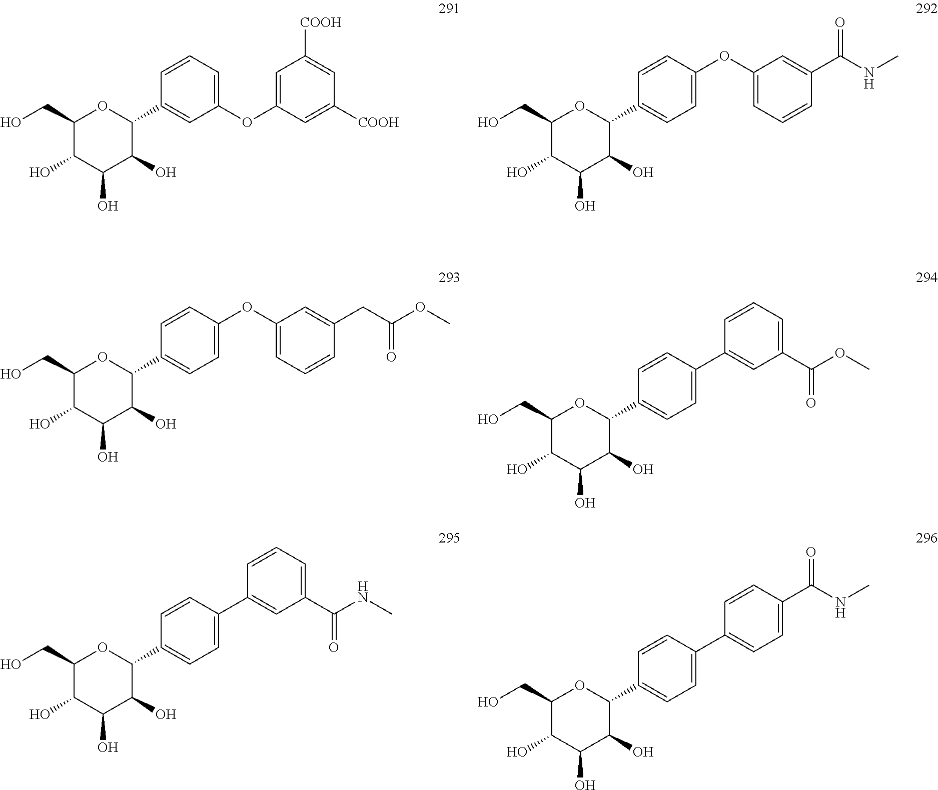 [SMILES: O=C(O)c1cc(Oc2cccc([C@H]3O[C@H](CO)[C@@H](O)[C@H](O)[C@@H]3O)c2)cc(C(=O)O)c1 ; CNC(=O)c1cccc(Oc2ccc([C@H]3O[C@H](CO)[C@@H](O)[C@H](O)[C@@H]3O)cc2)c1 ; COC(=O)Cc1cccc(Oc2ccc([C@H]3O[C@H](CO)[C@@H](O)[C@H](O)[C@@H]3O)cc2)c1 ; COC(=O)c1cccc(-c2ccc([C@H]3O[C@H](CO)[C@@H](O)[C@H](O)[C@@H]3O)cc2)c1 ; CNC(=O)c1cccc(-c2ccc([C@H]3O[C@H](CO)[C@@H](O)[C@H](O)[C@@H]3O)cc2)c1 ; CNC(=O)c1ccc(-c2ccc([C@H]3O[C@H](CO)[C@@H](O)[C@H](O)[C@@H]3O)cc2)cc1]

71. The compound of claim 70, selected from one or more of the following: Compound 48, 104, 105, 106, 107, 108, 111, 112, 120, 121, 125, 126, 127, 128, 131, 133, 136, 142, 150, 176, or 178.

72. The compound of claim 70, wherein the compound is selected from Compound 265 to Compound 290.

73. The compound of claim 70, wherein the compound is selected from Compound 1 to Compound 72 and Compound 291 to Compound 296.

74. A composition comprising the compound of claim 1 or a pharmaceutically acceptable salt thereof, and a pharmaceutically acceptable carrier, adjuvant, or vehicle.

75. A method of treating or preventing a bacteria infection in a subject, comprising administering to the subject an effective amount of the compound of claim 1 or a pharmaceutically acceptable salt thereof, or the composition of claim 74.

76. The method of claim 75, wherein the bacteria infection is urinary tract infection or inflammatory bowel disease.

77. The method of claim 75, wherein the bacteria infection is colitis.

78. The method of claim 75, wherein the bacteria infection is Crohn's disease.

79. A method of inhibiting FimH in a subject, comprising administering to the subject an effective amount of the compound of claim 1 or a pharmaceutically acceptable salt thereof, or the composition of claim 74.

80. The compound of claim 31, or a pharmaceutically acceptable salt thereof, wherein R₁ is optionally substituted phenyl.
